(19) United States
(12) Patent Application Publication
(10) Pub. No.: US 2025/0122500 A1
(43) Pub. Date: Apr. 17, 2025

(54) RNAi OLIGONUCLEOTIDE CONJUGATES

(71) Applicant: Dicerna Pharmaceuticals, Inc., Lexington, MA (US)

(72) Inventors: Bob Dale Brown, Littleton, MA (US); Maire Jung, Lincoln, MA (US); Travis Grim, Maynard, MA (US); Matthew Costales, Lexington, MA (US); Marc Abrams, Natick, MA (US); Martin Lee Koser, Westford, MA (US); Beata Kaminska, Waltham, MA (US); Jessica Lapierre, Lexington, MA (US)

(21) Appl. No.: 18/654,127

(22) Filed: May 3, 2024

Related U.S. Application Data
(63) Continuation-in-part of application No. PCT/US22/49230, filed on Nov. 8, 2022.
(63) Continuation-in-part of application No. PCT/US22/79301, filed on Nov. 4, 2022.
(63) Continuation-in-part of application No. PCT/US22/79302, filed on Nov. 4, 2022.
(60) Provisional application No. 63/340,291, filed on May 10, 2022.
(60) Provisional application No. 63/277,097, filed on Nov. 8, 2021.
(60) Provisional application No. 63/276,409, filed on Nov. 5, 2021.
(60) Provisional application No. 63/276,406, filed on Nov. 5, 2021.

(51) Int. Cl. C12N 15/113

(52) U.S. Cl. CPC C12N 15/113; C12N 2310/11; C12N 2310/315; C12N 2310/321; C12N 2310/322; C12N 2310/3515; C12N 2320/32

(57) ABSTRACT

Lipid-conjugated oligonucleotide are provided herein that inhibit or reduce expression of target genes. Also provided are compositions including the same and uses thereof, particularly uses relating to treating diseases, disorders and/or conditions associated with an RNAi trigger induced decrease in target gene expression.

### CROSS-REFERENCE TO RELATED APPLICATIONS

This application is a Continuation-in-Part of PCT/US2022/049230 filed Nov. 8, 2022, which claims the benefit of U.S. Provisional Patent Application No. 63/277,097 filed Nov. 8, 2021, and U.S. Provisional Patent Application No. 63/340,291 filed May 10, 2022, each of which are incorporated by reference herein in their entirety. This application is a Continuation-in-Part of PCT/US2022/079302 filed Nov. 4, 2022, which claims the benefit of U.S. Provisional Patent Application No. 63/276,406 filed Nov. 5, 2021, each of which are incorporated by reference herein in their entirety. This application is a Continuation-in-Part of PCT/US2022/079301 filed Nov. 4, 2022, which claims the benefit of U.S. Provisional Patent Application No. 63/276,409 filed Nov. 5, 2021, each of which are incorporated by reference herein in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 8, 2024, is named “DCY-10601.xml” and is 5,775,798 bytes in size. The sequence listing contained in this .XML file is part of the specification and is hereby incorporated by reference herein in its entirety.

### TECHNICAL FIELD

The disclosure relates to oligonucleotides linked to lipid moieties useful in the inhibition of target genes in a variety of tissues. Specifically, the present disclosure relates to oligonucleotide-lipid conjugates, methods to prepare them, their chemical configuration, and methods to modulate (e.g., inhibit or reduce) the expression of a target gene using the conjugated nucleic acids and oligonucleotides according to the description provided herein. The disclosure also provides pharmaceutically acceptable compositions comprising the conjugates of the present description and methods of using said compositions in the treatment of various diseases or disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligonucleotides (ASO), short interfering RNA (siRNA), double-stranded nucleic acids (dsNA), aptamers, ribozymes, exon-skipping and splice-altering oligonucleotides, immunomodulatory oligonucleotides, mRNAs, and CRISPR. Chemical modifications in the relevant molecules to allow functionality in various tissues, organs and/or cell types play a key role in overcoming challenges of oligonucleotide therapeutics, including improving nuclease stability, RNA-binding affinity, and pharmacokinetics. Various chemical modification strategies for oligonucleotides have been developed in the past three decades including modification of the sugars, nucleobases, and phosphodiester backbone to improve and optimize performance and therapeutic efficacy (Deleavey and Darma, CHEM. BIOL. 2012, 19(8):937-54; Wan and Seth, J. MED. CHEM. 2016, 59 (21): 9645-67; and Egli and Manoharan, ACC. CHEM. RES. 2019, 54 (4): 1036-47).

Dicer processed RNAi technologies utilize short double-stranded RNA (dsRNA) of approximately 21 base pair length with a two nucleotide (nt) 3′-overhang for the silencing of genes. These dsRNAs are generally called small interfering RNA (siRNA). SiRNA 12 to 22 nucleotides in length are the active agent in RNAi. The siRNA duplex serves as a guide for mRNA degradation. Upon siRNA incorporation into the RNA-induced silencing complex (RISC) the complex interacts with a specific mRNA and ultimately suppresses the mRNA signal. The sense strand or passenger strand of siRNA is typically cleaved at the 9th nucleotide downstream from the 5′-end of the sense strand by Argonaute 2 (Ago2) endonuclease. The activated RISC complex containing the antisense strand or guide strand binds to the target mRNA through Watson-Crick base pairing causing degradation or translational blocking of the targeted RNA.

However, the in vivo use of RNAi or siRNA molecules as pharmaceuticals has remained difficult due to obstacles encountered such as low biostability and unacceptable toxicity possibly caused by off-target effects. Various types of chemical modifications to improve the pharmacokinetics and to overcome bio-instability problems have been investigated over the years to improve the stability and specificity of the RNAi duplexes. In some cases, the chemical modification in siRNAs has improved the serum stability of siRNAs. However, often RNAi activity was lost, but the careful placement of some specific modified residues enables enhanced siRNA biostability without loss of siRNA potency. Some of these modifications have reduced siRNA side effects, such as the induction of recipient immune responses and inherent off-targeting effects and have even enhanced siRNA potency. Various chemically modified siRNAs have been investigated, among them were bridged nucleic acids (BNA's) such as 2′,4′-methylene bridged nucleic acid 2′,4′-BNAs, also known as locked nucleic acid or LNA's. Some of these modified siRNAs showed promising effects.

Therapeutic gene silencing mediated by RNAi oligonucleotide-based therapeutics comprising siRNAs or double-stranded nucleic acids (dsNAs) offer the potential for considerable expansion of the druggable target space and the possibility for treating orphan diseases that may be therapeutically unapproachable by other drug modalities (e.g., antibodies and/or small molecules). RNAi oligonucleotide-based therapeutics that inhibit or reduce expression of specific target genes in the liver have been developed and are currently in clinical use (Sehgal et al., (2013) JOURNAL OF HEPATOLOGY 59:1354-59). Technological hurdles remain for the development and clinical use of RNAi oligonucleotides in extrahepatic cells, tissues, and organs. Thus, an ongoing need exists in the art for the successful development of new and effective RNAi oligonucleotides to modulate the expression of a target genes in extrahepatic cells, tissues, and/or organs. This is complicated by the variant nature of the cell types in extrahepatic as well as concerns about circulatory patterns and cell membrane constituents such as receptor types.

Over the past decade, synthetic RNAi triggers such as double stranded RNAs have become ubiquitous tools in biological research, and extensive basic and clinical development efforts have recently culminated in the FDA approval of ONPATTRO™, the first RNAi drug. Despite a burgeoning drug development pipeline and an extensive compendium of excipients targeting ligands and delivery techniques, the difficulty of delivering RNAi agents to specific populations of disease related cells and or tissues, particularly outside the liver continues to limit the potential of RNAi therapy. Repeated attempts over the past several years to develop useful, active, and persistent RNAi agents and structures for use based on known liver delivery technology have not convincingly demonstrated the intended effects outside the liver. Thus, new dsRNA's with variant structures have been developed to overcome the limitations in the field.

### BRIEF SUMMARY OF THE DISCLOSURE

The present disclosure is based in part on the discovery of lipid-conjugated RNAi oligonucleotides that are capable of inhibiting expression of a target gene in hepatic and extrahepatic tissues. As demonstrated herein, lipid-conjugated RNAi oligonucleotides having a stem-loop at the 5′ end of the oligonucleotide showed comparable efficacy in reducing target gene expression in several regions of the central nervous system as a lipid-conjugated RNAi oligonucleotide having a stem-loop at the 3′ end of the oligonucleotide. Further, the presence of a stable stem-loop (e.g., UACG) improved reduction of target gene expression compared to a relatively less stable stem-loop (e.g., GAAA). In some aspects, the presence of a Tm-increasing nucleotide (e.g., locked nucleic acid) and/or truncation of a sense strand at the 3′ terminus improved reduction of target gene expression.

It is further shown herein that lipid-conjugated RNAi oligonucleotides having a “double-overhang” (e.g., an overhang of at least one nucleotide at each of the 5′ and 3′ termini of an antisense strand) reduced target gene expression in the central nervous system at comparable levels relative to an RNAi oligonucleotide having only one overhang, i.e., at the 3′ terminus of the antisense strand. Up to three nucleotide truncations at the 3′ terminus of the sense strand was tolerated, whereas introduction of a Tm-increasing nucleotide allowed for truncations of up to four nucleotides.

It has been demonstrated lipid-conjugated RNAi oligonucleotides delivered to the eye can effectively reduce expression of an ocular mRNA. Specifically, double-overhang RNAi oligonucleotides having a lipid conjugated to the 5′ terminal nucleotide of the sense strand reduced expression of a target gene in the optic nerve and retina.

Further shown herein, are lipid-conjugated RNAi oligonucleotides capable of reducing expression of a target gene in a macrophage of the liver. Specifically, RNAi oligonucleotides having a blunt end comprising the 3′ terminus of the sense strand and the 5′ terminus of the antisense strand, and an overhang of up to seven nucleotides on the 3′ terminus of the antisense strand, resulted in reduced expression of a gene expressed in macrophages. Further, lipid-conjugated RNAi oligonucleotides having a double-overhang, with or without Tm-increasing nucleotides, similarly reduced expression of the macrophage target gene. Macrophages make up about 33% of all liver cells and are believed to play a role in liver inflammation. Accordingly, without wishing to be bound by theory, the lipid-conjugated RNAi oligonucleotides disclosed herein are useful for targeting macrophages and treating liver diseases, including, but not limited to, drug or alcohol toxicity, steatosis, infection (e.g., viral infection), inflammatory liver diseases, fibrosis, hepatocellular carcinoma, and cirrhosis.

Also shown according to the current disclosure are the bases of the lipid-conjugated RNAi oligonucleotides modified by the addition of LNA, 2′-O-methyl modification, or phosphorothioate (PS) modification; either or both termini of the sense strand are modified with PS modification, 2′-O-methyl modification, or both; or the single strand overhang of the antisense sense strand is modified by LNA modification, 2′-O-methyl modification, PS modification, or any combination thereof. In some embodiments, the sense strand and the antisense strand of the RNAi trigger are modified by different chemical modifications.

Also shown herein are lipid-conjugated RNAi oligonucleotides having either a blunt end or a stem-loop at the 3′ terminus of the oligonucleotides, with truncated sense strands (e.g., having an overhang at the 3′ terminus of the antisense strand) and Tm-increasing nucleotides, are capable of reducing target gene expression in several tissues, including the liver, skeletal muscle, adipose and adrenal.

Without wishing to be bound by theory, the lipid-conjugated RNAi oligonucleotides described herein are useful for reducing expression of a target gene in both hepatic and extrahepatic tissues.

In some aspects, the disclosure provides a double-stranded oligonucleotide comprising an antisense strand of about 20-22 nucleotides in length and a sense strand of about 8-20 nucleotides in length, wherein the antisense and sense strands form a duplex region of about 8-20 base pairs, wherein the antisense strand comprises an orientation of 5′ to 3′, wherein the antisense strand comprises a 5′ overhang of at least one nucleotide and a 3′ overhang of at least one nucleotide, wherein the antisense strand comprises a region of complementarity to a mRNA target sequence, and wherein the sense strand comprises at least one lipid moiety conjugated to a nucleotide on the sense strand. In some aspects, the 5′ overhang is about 1-10 nucleotides. In some aspects, the 5′ overhang is about 2-10 nucleotides. In some aspects, the 5′ overhang is about 1-6 nucleotides. In some aspects, the 3′ overhang is about 2-8 nucleotides. In some aspects, the 3′ overhang is about 2-12 nucleotides. In some aspects, the 5′ overhang is 2 nucleotides and the 3′ overhang is about 3-7 nucleotides. In some aspects, the 3′ overhang is 2 nucleotides, and the 5′ overhang is about 2-8 nucleotides. In some aspects, the 3′ overhang is 6-8 nucleotides, and the 5′ overhang is about 2-4 nucleotides.

In any of the foregoing or related aspects,
(i) the sense strand is 18 nucleotides, the duplex region is 18 nucleotides, the 5′ overhang is 2 nucleotides and the 3′ overhang is 2 nucleotides;
(ii) the sense strand is 17 nucleotides, the duplex region is 17 nucleotides, the 5′ overhang is 3 nucleotides and the 3′ overhang is 2 nucleotides;
(iii) the sense strand is 16 nucleotides, the duplex region is 16 nucleotides, the 5′ overhang is 4 nucleotides and the 3′ overhang is 2 nucleotides; or
(iv) the sense strand is 13 nucleotides, the duplex region is 13 nucleotides, the 5′ overhang is 2 nucleotides and the 3′ overhang is 7 nucleotides.

In any of the foregoing or related aspects,
(i) the sense strand is 12 nucleotides, the duplex region is 12 nucleotides, the 5′ overhang is 2 nucleotides. and the 3′ overhang is 8 nucleotides;
(ii) the sense strand is 12 nucleotides, the duplex region is 12 nucleotides, the 5′ overhang is 3 nucleotides, and the 3′ overhang is 7 nucleotides; or
(iii) the sense strand is 10 nucleotides, the duplex region is 10 nucleotides, the 5′ overhang is 1 nucleotide, and the 3′ overhang is 11 nucleotides.

In some aspects, the sense strand is 13 nucleotides, the duplex region is 13 nucleotides, the 5′ overhang is 2 nucleotides, and the 3′ overhang is 7 nucleotides. In other aspects, the sense strand is 12 nucleotides, the duplex region is 12 nucleotides, the 5′ overhang is 2 nucleotides. and the 3′ overhang is 8 nucleotides. In other aspects, the sense strand is 12 nucleotides, the duplex region is 12 nucleotides, the 5′ overhang is 3 nucleotides, and the 3′ overhang is 7 nucleotides. In some aspects, the sense strand is 10 nucleotides, the duplex region is 10 nucleotides, the 5′ overhang is 1 nucleotide, and the 3′ overhang is 11 nucleotides.

In any of the foregoing or related aspects, the lipid moiety is selected from:

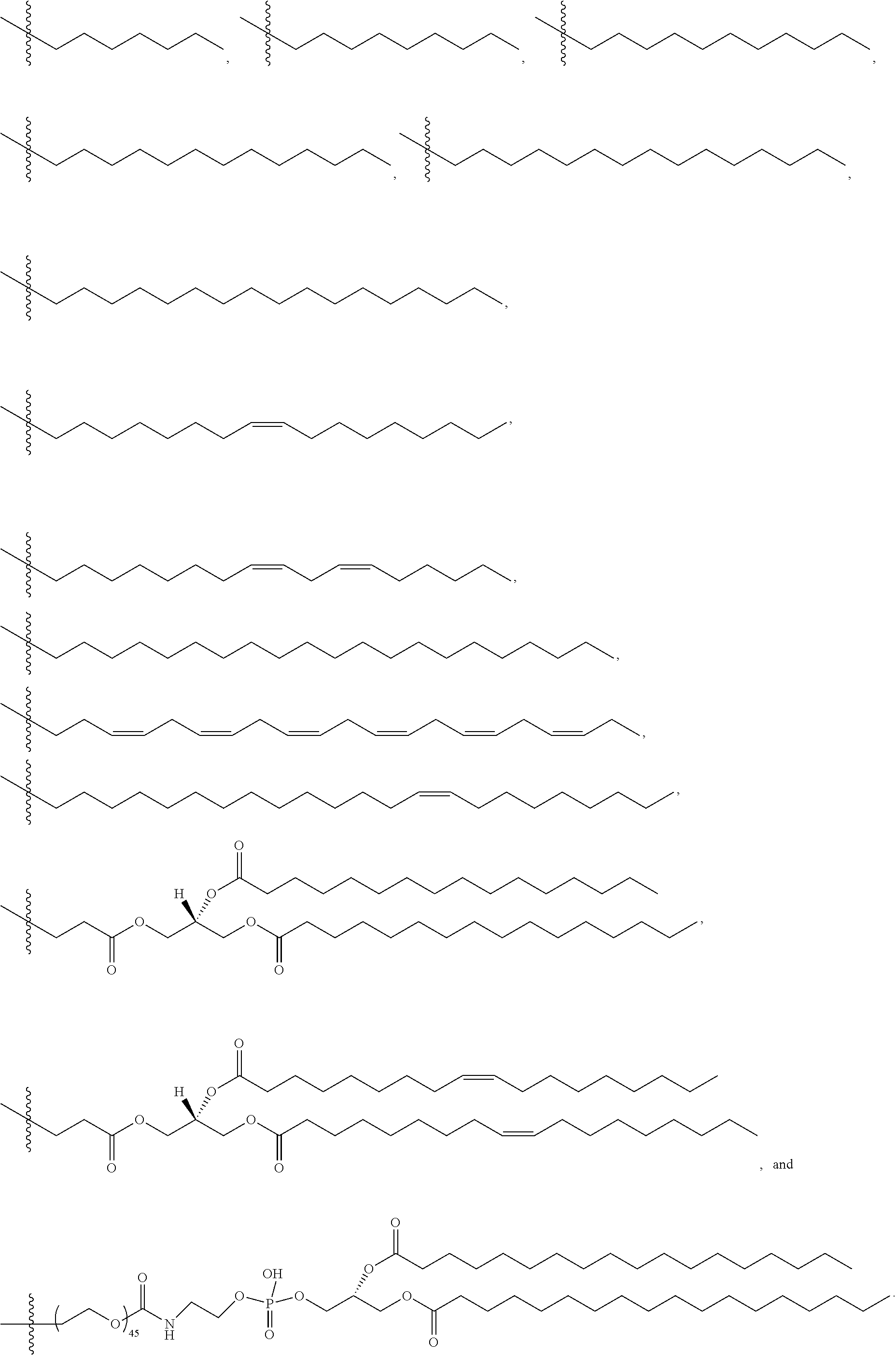

In some aspects, the lipid moiety is a hydrocarbon chain. In some aspects, the hydrocarbon chain is a C8-C30 hydrocarbon chain. In some aspects, the hydrocarbon chain is a C16 hydrocarbon chain. In some aspects, the C16 hydrocarbon chain is represented by

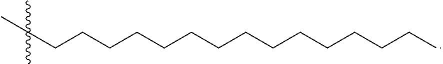 [SMILES: *CCCCCCCCCCCCCCC]

In some aspects, the hydrocarbon chain is a C22 hydrocarbon chain. In some aspects, the C22 hydrocarbon chain is represented by

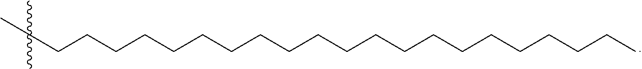 [SMILES: *CCCCCCCCCCCCCCCCCCCCC]

In any of the foregoing or related aspects, the lipid moiety is conjugated to the 5′ terminal nucleotide of the sense strand. In some aspects, the lipid moiety is conjugated to the 2′ carbon of the ribose ring of the nucleotide.

In any of the foregoing or related aspects, the antisense strand is 22 nucleotides, wherein positions are numbered 1-22 from 5′ to 3′. In some aspects, the nucleotide conjugated to the lipid moiety forms a base pair with a nucleotide at position 14 of the antisense strand, wherein positions are numbered 5′ to 3′. In some aspects, the nucleotide conjugated to the lipid moiety forms a base pair with a nucleotide at position 12, 14, or 16 of the antisense strand, wherein positions are numbered 5′ to 3′.

In any of the foregoing or related aspects, the region of complementarity is fully complementary to the mRNA target sequence. In other aspects, the region of complementarity is partially complementary to the mRNA target sequence. In some aspects, the region of complementarity comprises no more than four mismatches to the mRNA target sequence. In some aspects, the region of complementarity comprises up to four mismatches to the mRNA target sequence.

In some aspects, the region of complementarity comprises no more than four mismatches to the mRNA target sequence.

In any of the foregoing or related aspects, the mRNA target sequence is a central nervous system (CNS) target sequence. In some aspects, the CNS target sequence is a neuronal mRNA target sequence or an ocular mRNA target sequence. In some aspects, the mRNA target sequence is a liver mRNA target sequence. In some aspects, the liver mRNA target sequence is a liver macrophage mRNA target sequence. In some aspects, the liver mRNA target sequence is a liver hepatocyte mRNA target sequence. In some aspects, the liver mRNA target sequence is a liver sinusoidal endothelial cell mRNA target sequence. In other aspects, the mRNA target sequence is an ocular mRNA target sequence.

In any of the foregoing or related aspects, the oligonucleotide comprises at least one modified nucleotide. In some aspects, the modified nucleotide comprises a 2′-modification. In some aspects, each of the nucleotides of the sense strand and the antisense strand comprise a 2′-modification. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, the sense strand comprises nucleotide positions numbered 5′ to 3′, wherein each of positions 8-11 comprise a 2′-fluoro modification. In some aspects, the sense strand comprises a 2′-fluoro modification at each of nucleotides forming a base pair with nucleotides at positions 10-13 of the antisense strand, wherein positions are numbered 5′ to 3′. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects, the remaining nucleotides comprise a 2′-O-methyl modification, provided that the nucleotide of the sense strand conjugated to the at least one lipid moiety does not comprise a 2′-O-methyl modification.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the antisense strand comprises a phosphorothioate linkage between positions 13 and 14, and between positions 14 and 15. In some aspects, the sense strand comprises a phosphorothioate linkage between positions 1 and 2, between the penultimate nucleotide and third nucleotide from the 3′ end, and between the penultimate nucleotide and ultimate nucleotide.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate. In some aspects, the phosphorylated nucleotide is 4′-O-monomethylphosphonate-2′-O-methyl uridine.

In any of the foregoing or related aspects, the sense strand comprises at least one Tm-increasing nucleotide. In some aspects, the sense strand comprises up to four Tm-increasing nucleotides. In some aspects, the Tm-increasing nucleotide is a bicyclic nucleotide. In some aspects, the Tm-increasing nucleotide is a locked nucleic acid.

In other aspects, the disclosure provides a double-stranded oligonucleotide comprising an antisense strand of 22 nucleotides in length and a sense strand of 18 nucleotides in length, wherein the antisense and sense strands form a duplex region of 18 base pairs, wherein the antisense strand comprises an orientation of 5′ to 3′, wherein the antisense strand comprises a 5′ overhang comprising at least two nucleotides and a 3′ overhang comprising at least two nucleotides, wherein the antisense strand comprises a region of complementarity to a mRNA target sequence, wherein the sense strand comprises at least one lipid moiety conjugated to a 5′ terminal nucleotide of the sense strand, and wherein each of the antisense and sense strands comprise at least one 2′-modified nucleotide and at least one modified internucleotide linkage.

In some aspects, the sense strand comprises a 2′-fluoro modification at positions 8-11, numbered 5′ to 3′. In some aspects, the antisense strand comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification. In some aspects, the at least one modified internucleotide linkage is a phosphorothioate linkage. In some aspects, the sense strand comprises phosphorothioate linkages between nucleotides at positions 1 and 2, 16 and 17, and 17 and 18, numbered 5′ to 3′. In some aspects, the antisense strand comprises phosphorothioate linkages between nucleotides at positions 1 and 2, 2 and 3, 3 and 4, 20 and 21, and 21 and 22, numbered 5′ to 3′. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

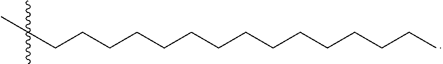 [SMILES: *CCCCCCCCCCCCCCC]

In some aspects, the lipid moiety is a C22 hydrocarbon represented by:

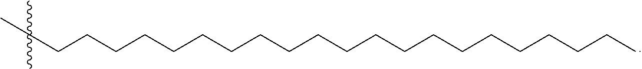 [SMILES: *CCCCCCCCCCCCCCCCCCCCC]

In some aspects, the lipid moiety is conjugated to the 2′ carbon of the ribose ring of the nucleotide. In some aspects, the 5′ overhang is 2 nucleotides and the 3′ overhang is 2 nucleotides. In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is a central nervous system (CNS) target sequence, optionally a neuronal mRNA target sequence or an ocular mRNA target sequence. In some aspects, the mRNA target sequence is a liver mRNA target sequence, optionally a liver macrophage mRNA target sequence, a liver hepatocyte mRNA target sequence, or a liver sinusoidal endothelial cell mRNA target sequence. In some aspects, the mRNA target sequence is an ocular mRNA target sequence. In some aspects, the mRNA target sequence is an astrocyte mRNA target sequence.

In some aspects, the disclosure provides a double-stranded oligonucleotide comprising an antisense strand of 22 nucleotides in length and a sense strand of 18 nucleotides in length, wherein the antisense and sense strands form a duplex region of 18 base pairs, wherein the antisense strand comprises an orientation of 5′ to 3′, wherein the antisense strand comprises a 5′ overhang comprising two nucleotides and a 3′ overhang comprising two nucleotides, wherein the antisense strand comprises a region of complementarity to a central nervous system mRNA target sequence, wherein the sense strand comprises at least one lipid moiety conjugated to a 5′ terminal nucleotide of the sense strand, wherein the sense strand comprises a 2′-fluoro modification at positions 8-11 and a 2′-O-methyl modification at positions 2-7 and 12-18, wherein the sense strand comprises a phosphorothioate linkage between positions 1 and 2, positions 16 and 17, and positions 17 and 18, wherein the antisense strand comprises a 2′-fluoro modification at positions 2-5, 7, 10 and 14 and a 2′-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In some aspects, the disclosure provides a double-stranded oligonucleotide comprising an antisense strand of 22 nucleotides in length and a sense strand of 18 nucleotides in length, wherein the antisense and sense strands form a duplex region of 18 base pairs, wherein the antisense strand comprises an orientation of 5′ to 3′, wherein the antisense strand comprises a 5′ overhang comprising two nucleotides and a 3′ overhang comprising two nucleotides, wherein the antisense strand comprises a region of complementarity to an ocular mRNA target sequence, wherein the sense strand comprises at least one lipid moiety conjugated to a 5′ terminal nucleotide of the sense strand, wherein the sense strand comprises a 2′-fluoro modification at positions 8-11 and a 2′-O-methyl modification at positions 2-7 and 12-18, wherein the sense strand comprises a phosphorothioate linkage between positions 1 and 2, positions 16 and 17, and positions 17 and 18, wherein the antisense strand comprises a 2′-fluoro modification at positions 2-5, 7, 10 and 14 and a 2′-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In some aspects, the disclosure provides a double-stranded oligonucleotide comprising an antisense strand of 22 nucleotides in length and a sense strand of 18 nucleotides in length, wherein the antisense and sense strands form a duplex region of 18 base pairs, wherein the antisense strand comprises an orientation of 5′ to 3′, wherein the antisense strand comprises a 5′ overhang comprising two nucleotides and a 3′ overhang comprising two nucleotides, wherein the antisense strand comprises a region of complementarity to a macrophage mRNA target sequence, wherein the sense strand comprises at least one lipid moiety conjugated to a 5′ terminal nucleotide of the sense strand, wherein the sense strand comprises a 2′-fluoro modification at positions 8-11 and a 2′-O-methyl modification at positions 2-7 and 12-18, wherein the sense strand comprises a phosphorothioate linkage between positions 1 and 2, positions 16 and 17, and positions 17 and 18, wherein the antisense strand comprises a 2′-fluoro modification at positions 2-5, 7, 10 and 14 and a 2′-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In other aspects, the disclosure provides a double-stranded oligonucleotide comprising an antisense strand of 22 nucleotides in length and a sense strand of 17 nucleotides in length, wherein the antisense and sense strands form a duplex region of 17 base pairs, wherein the antisense strand comprises an orientation of 5′ to 3′, wherein the antisense strand comprises a 5′ overhang comprising at least three nucleotides and a 3′ overhang comprising at least two nucleotides, wherein the antisense strand comprises a region of complementarity to a mRNA target sequence, wherein the sense strand comprises at least one lipid moiety conjugated to a 5′ terminal nucleotide of the sense strand, and wherein each of the antisense and sense strands comprise at least one 2′-modified nucleotide and at least one modified internucleotide linkage.

In some aspects, the sense strand comprises a 2′-fluoro modification at positions 8-11, numbered 5′ to 3′. In some aspects, the antisense strand comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification. In some aspects, the at least one modified internucleotide linkage is a phosphorothioate linkage. In some aspects, the sense strand comprises phosphorothioate linkages between nucleotides at positions 1 and 2, 15 and 16, and 16 and 17, numbered 5′ to 3′. In some aspects, the antisense strand comprises phosphorothioate linkages between nucleotides at positions 1 and 2, 2 and 3, 3 and 4, 20 and 21, and 21 and 22, numbered 5′ to 3′. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

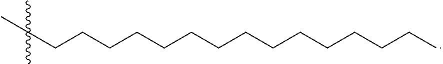 [SMILES: *CCCCCCCCCCCCCCC]

In some aspects, the lipid moiety is conjugated to the 2′ carbon of the ribose ring of the nucleotide. In some aspects, the 5′ overhang is 3 nucleotides and the 3′ overhang is 2 nucleotides. In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is a central nervous system (CNS) target sequence, optionally a neuronal mRNA target sequence.

In other aspects, the disclosure provides a double-stranded oligonucleotide comprising an antisense strand of 22 nucleotides in length and a sense strand of 17 nucleotides in length, wherein the antisense and sense strands form a duplex region of 17 base pairs, wherein the antisense strand comprises an orientation of 5′ to 3′, wherein the antisense strand comprises a 5′ overhang comprising three nucleotides and a 3′ overhang comprising two nucleotides, wherein the antisense strand comprises a region of complementarity to a central nervous system mRNA target sequence, wherein the sense strand comprises at least one lipid moiety conjugated to a 5′ terminal nucleotide of the sense strand, wherein the sense strand comprises a 2′-fluoro modification at positions 8-11 and a 2-O-methyl modification at positions 2-7 and 12-17, wherein the sense strand comprises phosphorothioate linkages between positions 1 and 2, positions 15 and 16, and positions 16 and 17, wherein the antisense strand comprises a 2′-fluoro modification at positions 2-5, 7, 10 and 14 and a 2-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In further aspects, the disclosure provides a double-stranded oligonucleotide comprising an antisense strand of 22 nucleotides in length and a sense strand of 16 nucleotides in length, wherein the antisense and sense strands form a duplex region of 16 base pairs, wherein the antisense strand comprises an orientation of 5′ to 3′, wherein the antisense strand comprises a 5′ overhang comprising at least four nucleotides and a 3′ overhang comprising at least two nucleotides, wherein the antisense strand comprises a region of complementarity to a mRNA target sequence, wherein the sense strand comprises at least one lipid moiety conjugated to a 5′ terminal nucleotide of the sense strand, wherein the sense strand comprises up to five Tm-increasing nucleotides, and wherein each of the antisense and sense strands comprise at least one 2′-modified nucleotide and at least one modified internucleotide linkage.

In some aspects, the sense strand comprises a 2′-fluoro modification at positions 8-11, numbered 5′ to 3′. In some aspects, the antisense strand comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification. In some aspects, the at least one modified internucleotide linkage is a phosphorothioate linkage. In some aspects, the sense strand comprises phosphorothioate linkages between nucleotides at positions 1 and 2, 14 and 15, and 15 and 16, numbered 5′ to 3′. In some aspects, the antisense strand comprises phosphorothioate linkages between nucleotides at positions 1 and 2, 2 and 3, 3 and 4, 20 and 21, and 21 and 22, numbered 5′ to 3′. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

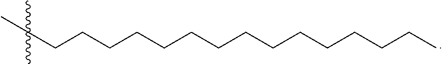 [SMILES: *CCCCCCCCCCCCCCC]

In some aspects, the lipid moiety is conjugated to the 2′ carbon of the ribose ring of the nucleotide. In some aspects, the 5′ overhang is 4 nucleotides and the 3′ overhang is 2 nucleotides. In some aspects, the sense strand comprises 1-5, 1-4, 1-3, or 1-2 Tm-increasing nucleotides. In some aspects, the sense strand comprises 1, 2, 3, 4 or 5 Tm-increasing nucleotides. In some aspects, the sense strand comprises up to three Tm-increasing nucleotides. In some aspects, the sense strand comprises a Tm-increasing nucleotide at positions 2, 15 and 16, numbered 5′ to 3′. In some aspects, the Tm-increasing nucleotide is a bicyclic nucleotide, optionally, a locked nucleic acid (LNA). In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is a central nervous system (CNS) target sequence, optionally a neuronal mRNA target sequence or an ocular mRNA target sequence.

In further aspects, the disclosure provides a double-stranded oligonucleotide comprising an antisense strand of 22 nucleotides in length and a sense strand of 16 nucleotides in length, wherein the antisense and sense strands form a duplex region of 16 base pairs, wherein the antisense strand comprises an orientation of 5′ to 3′, wherein the antisense strand comprises a 5′ overhang comprising at least four nucleotides and a 3′ overhang comprising at least two nucleotides, wherein the antisense strand comprises a region of complementarity to a central nervous system mRNA target sequence, wherein the sense strand comprises at least one lipid moiety conjugated to a 5′ terminal nucleotide of the sense strand, wherein the sense strand comprises a Tm-increasing nucleotide at positions 2, 15 and 16, wherein the sense strand comprises a 2′-fluoro modification at positions 8-11 and a 2-O-methyl modification at positions 3-7 and 12-13, wherein the sense strand comprises phosphorothioate linkages between positions 1 and 2, positions 14 and 15, and positions 15 and 16, wherein the antisense strand comprises a 2′-fluoro modification at positions 2-5, 7, 10 and 14 and a 2-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In yet further aspects, the disclosure provides a double-stranded oligonucleotide comprising an antisense strand of 22 nucleotides in length and a sense strand of 13 nucleotides in length, wherein the antisense and sense strands form a duplex region of 13 base pairs, wherein the antisense strand comprises an orientation of 5′ to 3′, wherein the antisense strand comprises a 5′ overhang comprising at least two nucleotides and a 3′ overhang comprising at least seven nucleotides, wherein the antisense strand comprises a region of complementarity to a mRNA target sequence, wherein the sense strand comprises at least one lipid moiety conjugated to an internal nucleotide of the sense strand, wherein the sense strand comprises up to three Tm-increasing nucleotides, and wherein each of the antisense and sense strands comprise at least one 2′-modified nucleotide and at least one modified internucleotide linkage.

In some aspects, the sense strand comprises a 2′-fluoro modification at positions 3-6, numbered 5′ to 3′. In some aspects, the antisense strand comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification. In some aspects, the at least one modified internucleotide linkage is a phosphorothioate linkage. In some aspects, the sense strand comprises phosphorothioate linkages between nucleotides at positions 1 and 2, 11 and 12, and 12 and 13, numbered 5′ to 3′. In some aspects, the antisense strand comprises phosphorothioate linkages between nucleotides at positions 1 and 2, 2 and 3, 3 and 4, 20 and 21, and 21 and 22, numbered 5′ to 3′. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is conjugated at a nucleotide at position 2 of the sense strand, numbered 5′ to 3′. In some aspects, the lipid moiety is a C22 hydrocarbon represented by:

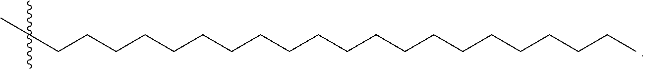 [SMILES: *CCCCCCCCCCCCCCCCCCCCC]

In some aspects, the lipid moiety is conjugated to the 2′ carbon of the ribose ring of the nucleotide. In some aspects, the 5′ overhang is 2 nucleotides and the 3′ overhang is 7 nucleotides. In some aspects, the sense strand comprises 1-3, or 1-2 Tm-increasing nucleotides. In some aspects, the sense strand comprises 1, 2, or 3 Tm-increasing nucleotides. In some aspects, the sense strand comprises a Tm-increasing nucleotide at (i) positions 1 and 10, or (ii) positions 1, 10 and 11, numbered 5′ to 3′. In some aspects, the Tm-increasing nucleotide is a bicyclic nucleotide, optionally a locked nucleic acid (LNA). In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is a liver target sequence. In some aspects, the mRNA target sequence is a hepatocyte target sequence. In some aspects, the mRNA target sequence is a liver sinusoidal endothelial cell mRNA target sequence. In some aspects, the mRNA target sequence is a macrophage mRNA target sequence, optionally a liver macrophage mRNA target sequence.

In yet further aspects, the disclosure provides a double-stranded oligonucleotide comprising an antisense strand of 22 nucleotides in length and a sense strand of 13 nucleotides in length, wherein the antisense and sense strands form a duplex region of 13 base pairs, wherein the antisense strand comprises an orientation of 5′ to 3′, wherein the antisense strand comprises a 5′ overhang comprising two nucleotides and a 3′ overhang comprising seven nucleotides, wherein the antisense strand comprises a region of complementarity to a macrophage mRNA target sequence, wherein the sense strand comprises at least one lipid moiety conjugated to a nucleotide at position 2 of the sense strand, wherein the sense strand comprises a Tm-increasing nucleotide at positions 1, and 11, wherein the sense strand comprises a 2′-fluoro modification at positions 3-6 and a 2-O-methyl modification at positions 7-10 and 12-13, wherein the sense strand comprises phosphorothioate linkages between positions 1 and 2, positions 11 and 12, and positions 12 and 13, wherein the antisense strand comprises a 2′-fluoro modification at positions 2-5, 7, 10 and 14 and a 2-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In some aspects, the disclosure provides a double-stranded oligonucleotide comprising an antisense strand of 22 nucleotides in length and a sense strand of 13 nucleotides in length, wherein the antisense and sense strands form a duplex region of 13 base pairs, wherein the antisense strand comprises an orientation of 5′ to 3′, wherein the antisense strand comprises a 5′ overhang comprising two nucleotides and a 3′ overhang comprising seven nucleotides, wherein the antisense strand comprises a region of complementarity to a macrophage mRNA target sequence, wherein the sense strand comprises at least one lipid moiety conjugated to a nucleotide at position 2 of the sense strand, wherein the sense strand comprises a Tm-increasing nucleotide at positions 1, 10, and 11, wherein the sense strand comprises a 2′-fluoro modification at positions 3-6 and a 2-O-methyl modification at positions 7-19 and 12-13, wherein the sense strand comprises phosphorothioate linkages between positions 1 and 2, positions 11 and 12, and positions 12 and 13, wherein the antisense strand comprises a 2′-fluoro modification at positions 2-5, 7, 10 and 14 and a 2-O-methyl modification at positions 1, 6, 8, 9, 11-13, and 15-22, wherein the antisense strand comprises phosphorothioate linkages between positions 1 and 2, positions 2 and 3, positions 3 and 4, positions 20 and 21, and positions 21 and 22, and wherein the antisense strand comprises a 5′ terminal phosphorylated uridine.

In some aspects, the disclosure provides a double-stranded oligonucleotide comprising an antisense strand of 22 nucleotides in length and a sense strand of 12 nucleotides in length, wherein the antisense and sense strands form a duplex region of 12 base pairs, wherein the antisense strand comprises an orientation of 5′ to 3′, wherein the antisense strand comprises a 5′ overhang comprising at least two nucleotides and a 3′ overhang comprising at least seven nucleotides, wherein the antisense strand comprises a region of complementarity to an mRNA target sequence, wherein the sense strand comprises at least one lipid moiety conjugated to a 5′ terminal nucleotide of the sense strand, and wherein each of the antisense and sense strands comprise at least one 2′-modified nucleotide and at least one modified internucleotide linkage. In some aspects, the sense strand comprises a 2′-fluoro modification at positions 3-6 or 4-7, numbered 5′ to 3′. In some aspects, the at least one modified internucleotide linkage is a phosphorothioate linkage. In some aspects, the sense strand comprises phosphorothioate linkages between nucleotides at positions 1 and 2, 10 and 11, and 11 and 12, numbered 5′ to 3′. In some aspects, the antisense strand comprises phosphorothioate linkages between nucleotides at positions 1 and 2, 2 and 3, 3 and 4, 12 and 14, 14 and 15, 20 and 21, and 21 and 22, numbered 5′ to 3′. In some aspects, the 5′ overhang is 2 nucleotides and the 3′ overhang is 8 nucleotides. In some aspects, the 5′ overhang is 3 nucleotides and the 3′ overhang is 7 nucleotides. In some aspects, the oligonucleotide comprises (i) 1-3 or 1-2 Tm-increasing nucleotides, or (ii) 1, 2 or 3 Tm-increasing nucleotides. In some aspects, the sense strand comprises a Tm-increasing nucleotide at (i) positions 2, 10 and 11, or (ii) positions 2, 11 and 12, numbered 5′ to 3′. In some aspects, the Tm-increasing nucleotide is a bicyclic nucleotide, optionally a locked nucleic acid (LNA).

In other aspects, the disclosure provides a double-stranded oligonucleotide comprising an antisense strand of 22 nucleotides in length and a sense strand of 10 nucleotides in length, wherein the antisense and sense strands form a duplex region of 10 base pairs, wherein the antisense strand comprises an orientation of 5′ to 3′, wherein the antisense strand comprises a 5′ overhang comprising one nucleotide and a 3′ overhang comprising eleven nucleotides, wherein the antisense strand comprises a region of complementarity to an mRNA target sequence, wherein the sense strand comprises at least one lipid moiety conjugated to a 5′ terminal nucleotide of the sense strand, and wherein each of the antisense and sense strands comprise at least one 2′-modified nucleotide and at least one modified internucleotide linkage. In some aspects, the at least one modified internucleotide linkage is a phosphorothioate linkage. In some aspects, the sense strand comprises phosphorothioate linkages between nucleotides at positions 1 and 2, 8 and 9, and 9 and 10, numbered 5′ to 3′. In some aspects, the antisense strand comprises phosphorothioate linkages between nucleotides at positions 1 and 2, 2 and 3, 3 and 4, 12 and 14, 14 and 15, 20 and 21, and 21 and 22, numbered 5′ to 3′. In some aspects, the oligonucleotide comprises (i) 1-3 Tm-increasing nucleotides, or (ii) 1, 2 or 3 Tm-increasing nucleotides. In some aspects, the sense strand comprises a Tm-increasing nucleotide at positions 2, 6 and 7. In some aspects, the Tm-increasing nucleotide is a bicyclic nucleotide, optionally a locked nucleic acid (LNA).

In any of the foregoing or related aspects, the antisense strand comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification

In any of the foregoing or related aspects, the oligonucleotide is a Dicer substrate. In some aspects, the oligonucleotide reduces expression of the mRNA target sequence in a cell or population of cells in vitro and/or in vivo.

In some aspects, the disclosure provides a pharmaceutical composition comprising an oligonucleotide described herein, and a pharmaceutically acceptable carrier, delivery agent or excipient.

In other aspects, the disclosure provides a method for treating a subject having a disease, disorder or condition associated with expression of a target mRNA, the method comprising administering to the subject a therapeutically effective amount of an oligonucleotide or pharmaceutical composition described herein. In some aspects, the target mRNA is expressed in the central nervous system, optionally wherein the central nervous system comprises the frontal cortex, hippocampus, medulla, cerebellum, lumbar dorsal root ganglion, and/or lumbar spinal cord. In some aspects, the target mRNA is expressed in a neuron of the central nervous system. In some aspects, the target mRNA is expressed in a macrophage. In some aspects, the macrophage is in the liver. In some aspects, the target mRNA is expressed in the liver. In some aspects, the target mRNA is expressed in a hepatocyte. In some aspects, the target mRNA is expressed in a liver sinusoidal endothelial cell. In some aspects, the target mRNA is expressed in ocular tissue. In some aspects, the target mRNA is expressed in a tissue of the central nervous system, liver tissue, ocular tissue, adipose tissue, muscle tissue, adrenal tissue, cardiac tissue, lung tissue, or any combination thereof.

In some aspects, the disclosure provides a method for treating a subject having a disease, disorder or condition associated with expression of an mRNA of the central nervous system, the method comprising administering to the subject a therapeutically effective amount of an oligonucleotide or pharmaceutical composition described herein, optionally wherein the central nervous system comprises the frontal cortex, hippocampus, medulla, cerebellum, lumbar dorsal root ganglion, and/or lumbar spinal cord. In some aspects, the mRNA of the central nervous system is a neuronal mRNA.

In other aspects, the disclosure provides a method for treating a subject having a disease, disorder or condition associated with expression of an mRNA of the liver, the method comprising administering to the subject a therapeutically effective amount of an oligonucleotide or pharmaceutical composition described herein.

In further aspects, the disclosure provides a method for treating a subject having a disease, disorder or condition associated with expression of an ocular mRNA, the method comprising administering to the subject a therapeutically effective amount of an oligonucleotide or pharmaceutical composition described herein.

In yet further aspects, the disclosure provides a method for treating a subject having a disease, disorder or condition associated with expression of a macrophage mRNA, the method comprising administering to the subject a therapeutically effective amount of an oligonucleotide or pharmaceutical composition described herein. In some aspects, the macrophage mRNA is expressed in the liver.

In some aspects, the disclosure provides a method of delivering an oligonucleotide to a cell or population of cells in the central nervous system, liver tissue or ocular tissue, the method comprising administering a pharmaceutical composition described herein.

In further aspects, the disclosure provides a method of reducing expression of a target mRNA in a subject, comprising administering to the subject an oligonucleotide or pharmaceutical composition described herein. In some aspects, the target mRNA is expressed in the central nervous system, optionally wherein the central nervous system comprises the frontal cortex, hippocampus, medulla, cerebellum, lumbar dorsal root ganglion, and/or lumbar spinal cord. In some aspects, the target mRNA is expressed in a neuron of the central nervous system. In some aspects, the target mRNA is expressed in the liver. In some aspects, the target mRNA is expressed in a hepatocyte. In some aspects, the target mRNA is expressed in a liver sinusoidal endothelial cell. In some aspects, the target mRNA is expressed in a macrophage. In some aspects, the macrophage is in the liver. In some aspects, the target mRNA is expressed in ocular tissue.

In other aspects, the disclosure provides a kit comprising an oligonucleotide described herein, an optional pharmaceutically acceptable carrier, and a package insert comprising instructions for administration to a subject having a disease, disorder or condition associated with the overexpression of a target mRNA. In further aspects, the disclosure provides a kit comprising an oligonucleotide described herein, an optional pharmaceutically acceptable carrier, and a package insert comprising instructions for administration to a subject having a disease, disorder or condition associated with the reduction in expression of a target mRNA.

In some aspects, the disclosure provides use of an oligonucleotide or pharmaceutical composition described herein, in the manufacture of a medicament for the treatment of a disease, disorder, or condition associated with the reduction in the expression of a target mRNA. In other aspects, the disclosure provides use of an oligonucleotide or pharmaceutical composition described herein, in the manufacture of a medicament for the treatment of a disease, disorder, or condition associated with the overexpression of a target mRNA. In further aspects, the disclosure provides an oligonucleotide or pharmaceutical composition described herein, for use, or adaptable for use, in the treatment of a disease, disorder, or condition associated with expression of a target mRNA.

In any of the foregoing or related aspects, the target mRNA is expressed in the central nervous system, a neuron of the central system, the liver, a macrophage, optionally a macrophage in the liver, ocular tissue, or any combination thereof, optionally wherein the central nervous system comprises the frontal cortex, hippocampus, medulla, cerebellum, lumbar dorsal root ganglion, and/or lumbar spinal cord.

In some aspects, the disclosure provides a method of activating target-specific RNA interference (RNAi) in an organism comprising administering to said organism an oligonucleotide described herein, said oligonucleotide being administered in an amount sufficient for degradation of the target mRNA to occur, thereby activating target-specific RNAi in the organism. In some aspects, the target mRNA specifies the amino acid sequence of a protein involved or predicted to be involved in a human disease or disorder. In some aspects, the disease or disorder is selected from the group consisting of viral infections, bacterial infections, parasitic infections, cancers, allergies, autoimmune diseases, immunodeficiencies, and immunosuppression.

In some aspects, the disclosure provides a double-stranded oligonucleotide comprising an antisense strand of about 15-30 nucleotides in length and a sense strand of about 15-50 nucleotides in length, wherein the antisense and sense strands form a duplex region of about 15-30 base pairs, wherein the antisense strand comprises a region of complementarity to a mRNA target sequence, wherein the sense strand comprises (i) at least one lipid moiety conjugated to a nucleotide of the sense strand, and (ii) a stem-loop, wherein the stem-loop comprises a nucleotide sequence represented by the formula: 5′-S1-L-S2-3′, wherein S1 is complementary to S2, and wherein L forms a loop between S1 and S2, and wherein the sense and antisense strands each comprise an orientation of 5′ to 3′, and wherein the stem-loop is at the 5′ end of the sense strand.

In any of the foregoing or related aspects, the oligonucleotide comprises a blunt end. In some aspects, the blunt end comprises the 3′ end of the sense strand and the 5′ end of the antisense strand.

In some aspects, the oligonucleotide comprises an overhang of at least two nucleotides. In some aspects, the overhang comprises the 5′ end of the antisense strand.

In some aspects, the sense strand is about 28-38 nucleotides. In some aspects, the antisense strand is 22 nucleotides. In some aspects, the lipid moiety is conjugated to a nucleotide comprising the loop.

In any of the foregoing or related aspects,
(i) the sense strand is 28 nucleotides, and the lipid moiety is conjugated to a nucleotide at position 4, positions numbered 5′ to 3′;
(ii) the sense strand is 30 nucleotides, and the lipid moiety is conjugated to a nucleotide at position 4, positions numbered 5′ to 3′;
(iii) the sense strand is 34 nucleotides, and the lipid moiety is conjugated to a nucleotide at position 6 or position 15, positions numbered 5′ to 3′; or
(iv) the sense strand is 38 nucleotides, and the lipid moiety is conjugated to a nucleotide at position 8, positions numbered 5′ to 3′.

In any of the foregoing or related aspects, the lipid moiety is selected from:

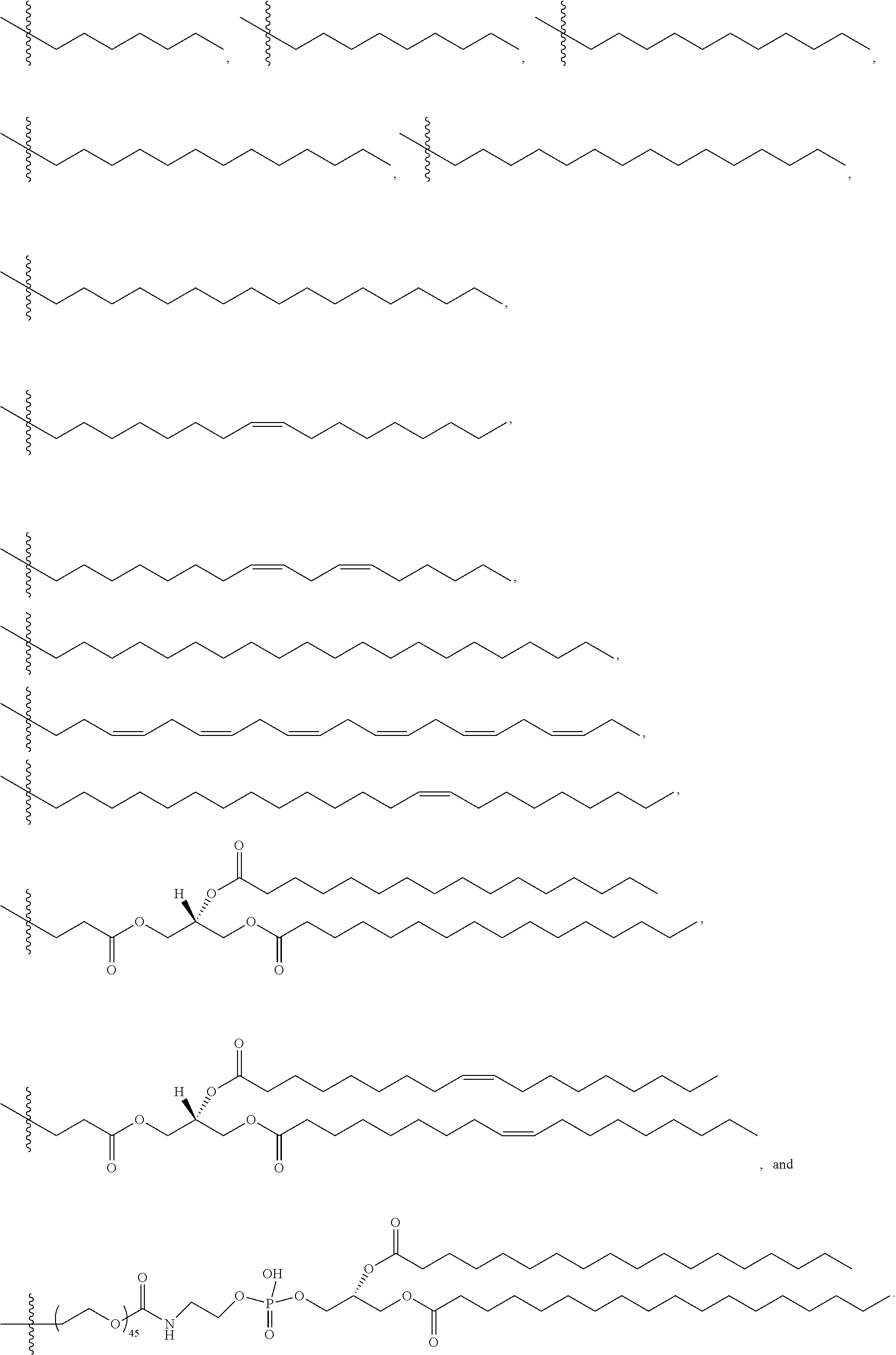

In some aspects, the lipid moiety is a hydrocarbon chain. In some aspects, the hydrocarbon chain is a C8-C30 hydrocarbon chain. In some aspects, the hydrocarbon chain is a C16 hydrocarbon chain. In some aspects, the C16 hydrocarbon chain is represented by

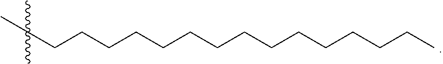 [SMILES: *CCCCCCCCCCCCCCC]

In any of the foregoing or related aspects, the lipid moiety is conjugated to the 2′ carbon of the ribose ring of the nucleotide.

In any of the foregoing or related aspects, the region of complementarity is fully complementary to the mRNA target sequence. In other aspects, the region of complementarity is partially complementary to the mRNA target sequence. In some aspects, the region of complementarity comprises no more than four mismatches to the mRNA target sequence.

In any of the foregoing or related aspects, the mRNA target sequence is a central nervous system (CNS) target sequence, optionally a neuronal mRNA target sequence or an ocular mRNA target sequence. In some aspects, the mRNA target sequence is an ocular mRNA target sequence.

In any of the foregoing or related aspects, the loop sequence is 5′-GAAA-3′. In some aspects, the loop sequence is 5′-UNCG-3′, wherein N is any nucleotide. In some aspects, the loop sequence is 5′-UACG-3′.

In any of the foregoing or related aspects, the oligonucleotide comprises at least one modified nucleotide. In some aspects, the modified nucleotide comprises a 2′-modification. In some aspects, each of the nucleotides of the sense strand and the antisense strand comprise a 2′-modification. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects,
(i) the sense strand is 38 nucleotides with positions 1-38 from 5′ to 3′, and wherein each of positions 26-29 comprise a 2′-fluoro modification;
(ii) the sense strand is 34 nucleotides with positions 1-34 from 5′ to 3′, and wherein each of positions 22-25 comprise a 2′-fluoro modification;
(iii) the sense strand is 30 nucleotides with positions 1-30 from 5′ to 3′, and wherein each of positions 18-21 comprise a 2′-fluoro modification; or
(iv) the sense strand is 28 nucleotides with positions 1-28 from 5′ to 3′, and wherein each of positions 18-21 comprise a 2′-fluoro modification.

In any of the foregoing or related aspects, comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the sense strand comprises a phosphorothioate linkage between the penultimate nucleotide and third nucleotide from the 3′ end, and between the penultimate nucleotide and ultimate nucleotide. In some aspects,
(i) the sense strand is 38 nucleotides with positions 1-38 from 5′ to 3′, and wherein the sense strand comprises a phosphorothioate linkage between positions 36 and 37, and 37 and 38;
(ii) the sense strand is 34 nucleotides with positions 1-34 from 5′ to 3′, and wherein the sense strand comprises a phosphorothioate linkage between positions 32 and 33, and 33 and 34;
(iii) the sense strand is 30 nucleotides with positions 1-30 from 5′ to 3′, and wherein the sense strand comprises a phosphorothioate linkage between positions 28 and 29, and 29 and 30; or
(iv) the sense strand is 28 nucleotides with positions 1-28 from 5′ to 3′, and wherein the sense strand comprises a phosphorothioate linkage between positions 26 and 27, and 27 and 28.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate. In some aspects, the phosphorylated nucleotide is 4′-O-monomethylphosphonate-2′-O-methyl uridine.

In any of the foregoing or related aspects, the sense strand comprises at least one Tm-increasing nucleotide. In some aspects, the sense strand comprises 1-6, 1-5, 1-4, 1-3 or 12 Tm-increasing nucleotides. In some aspects, the sense strand comprises 1, 2, 3, 4, 5 or 6 Tm-increasing nucleotides. In some aspects, the sense strand comprises up to six Tm-increasing nucleotides. In some aspects, the Tm-increasing nucleotide is a bicyclic nucleotide. In some aspects, the Tm-increasing nucleotide is a locked nucleic acid.

In any of the foregoing or related aspects, S1 and S2 each comprise 1-6 nucleotides. In some aspects, S1 and S2 each comprise 4 nucleotides. In some aspects, S1 and S2 each comprise 2 nucleotides. In some aspects, S1 and S2 each comprise at least one Tm-increasing nucleotide. In some aspects, S1 and S2 are each 4 nucleotides, wherein 1-3 nucleotides of each S1 and S2 are Tm-increasing nucleotides.

In any of the foregoing or related aspects, the remaining nucleotides comprise a 2′-O-methyl modification, provided that the nucleotide of the sense strand conjugated to the at least one lipid moiety does not comprise a 2′-O-methyl modification.

In some aspects, the disclosure provides, a double-stranded oligonucleotide comprising an antisense strand of about 20-22 nucleotides in length and a sense strand of about 32-34 nucleotides in length, wherein the antisense and sense strands form a duplex region of about 20-22 base pairs and the oligonucleotide is blunt ended, wherein the antisense strand comprises a region of complementarity to a mRNA target sequence, wherein the sense strand comprises (i) a stem-loop, wherein the stem-loop comprises a nucleotide sequence represented by the formula: 5′-S1-L-S2-3′, wherein S1 is complementary to S2, and wherein L forms a loop between S1 and S2, and (ii) at least one lipid moiety conjugated to a nucleotide of the loop, wherein the sense and antisense strands each comprise an orientation of 5′ to 3′, wherein the stem-loop is at the 5′ end of the sense strand, and wherein each of the antisense and sense strands comprise at least one 2′-modified nucleotide and at least one modified internucleotide linkage.

In some aspects, the sense strand is 34 nucleotides and comprises a 2′-fluoro modification at positions 22-25, numbered 5′ to 3′. In some aspects, the antisense strand is 22 nucleotides and comprises a 2′-fluoro modification at positions 2-5, 7, 10 and 14, numbered 5′ to 3′. In some aspects, the remaining nucleotides comprise a 2′-O-methyl modification, provided that the nucleotide of the sense strand conjugated to the at least one lipid moiety does not comprise a 2′-O-methyl modification. In some aspects, the at least one modified internucleotide linkage is a phosphorothioate linkage. In some aspects, the sense strand is 34 nucleotides and comprises phosphorothioate linkages between nucleotides at positions 1 and 2, 2 and 3, 32 and 33, and 33 and 34, numbered 5′ to 3′. In some aspects, the antisense strand is 22 nucleotides and comprises phosphorothioate linkages between nucleotides at positions 1 and 2, 2 and 3, 3 and 4, 20 and 21, and 21 and 22, numbered 5′ to 3′. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

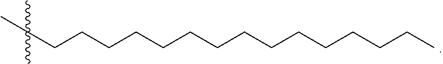 [SMILES: *CCCCCCCCCCCCCCC]

In some aspects, the lipid moiety is conjugated to the 2′ carbon of the ribose ring of the nucleotide. In some aspects, the lipid moiety is conjugated to a nucleotide at position 6 of the sense strand, numbered 5′ to 3′. In some aspects, S1 and S2 each comprise 1-6 nucleotides. In some aspects, S1 and S2 are each 4 nucleotides. In some aspects, L is 4 nucleotides. In some aspects, L comprises the sequence 5′-GAAA-3′. In some aspects, the region of complementarity is fully complementary to the mRNA target sequence. In other aspects, wherein the region of complementarity is partially complementary to the mRNA target sequence. In some aspects, the region of complementarity comprises no more than four mismatches to the mRNA target sequence. In some aspects, the mRNA target sequence is a central nervous system (CNS) target sequence, optionally a neuronal mRNA target sequence or an ocular mRNA target sequence.

In some aspects, the disclosure provides, a double-stranded oligonucleotide comprising an antisense strand of 22 nucleotides in length and a sense strand of 34 nucleotides in length, wherein the antisense and sense strands form a duplex region of 22 base pairs and the oligonucleotide is blunt ended, wherein the antisense strand comprises a region of complementarity to a central nervous system mRNA target sequence, wherein the sense strand comprises (i) a stem-loop, wherein the stem-loop comprises a nucleotide sequence represented by the formula: 5′-S1-L-S2-3′, wherein S1 is complementary to S2, wherein S1 and S2 each comprise 4 nucleotides, wherein L forms a loop between S1 and S2, and wherein L comprises four nucleotides, and (ii) at least one lipid moiety conjugated to a nucleotide of the loop, wherein the sense and antisense strands each comprise an orientation of 5′ to 3′, wherein the stem-loop is at the 5′ end of the sense strand, wherein the sense strand comprises a 2′-fluoro modification at positions 22-25 and a 2′-O-methyl modification at positions 1-5, 7-21 and 26-34, wherein the antisense strand comprises a 2′-fluoro modification at positions 2-5, 7, 10 and 14 and a 2-O-methyl modification at positions 1, 6, 8, 9, 11-13, and 15-22, wherein the sense strand comprises phosphorothioate linkages between positions 1 and 2, 2 and 3, 32 and 33, and 33 and 34, and wherein the antisense strand comprises phosphorothioate linkages between positions 1 and 2, 2 and 3, 3 and 4, 20 and 21, and 21 and 22.

In some aspects, the disclosure provides a double-stranded oligonucleotide comprising an antisense strand of about 20-22 nucleotides in length and a sense strand of about 26-28 nucleotides in length, wherein the antisense and sense strands form an asymmetric duplex region of about 20-22 base pairs comprising a 3′ terminal overhang of at least 2 nucleotides of the antisense strand, wherein the antisense strand comprises a region of complementarity to a mRNA target sequence, wherein the sense strand comprises: (i) a stem-loop, wherein the stem-loop comprises a nucleotide sequence represented by the formula: 5′-S1-L-S2-3′, wherein S1 is complementary to S2, wherein L forms a loop between S1 and S2 and comprises the sequence UNCG, and wherein S1 and S2 each comprise at least one Tm-increasing nucleotide, and (ii) at least one lipid moiety conjugated to a nucleotide of the loop, wherein the sense strand comprises an orientation of 5′ to 3′, wherein the stem-loop is at the 5′ end of the sense strand, and wherein each of the antisense and sense strands comprise at least one 2′-modified nucleotide and at least one modified internucleotide linkage.

In some aspects, the sense strand is 28 nucleotides and comprises a 2′-fluoro modification at positions 18-21, numbered 5′ to 3′. In some aspects, the antisense strand is 22 nucleotides and comprises a 2′-fluoro modification at positions 2-5, 7, 10 and 14, numbered 5′ to 3′. In some aspects, the at least one modified internucleotide linkage is a phosphorothioate linkage. In some aspects, the sense strand is 28 nucleotides and comprises phosphorothioate linkages between nucleotides at positions 26 and 27, and 27 and 28, numbered 5′ to 3′. In some aspects, the antisense strand is 22 nucleotides and comprises phosphorothioate linkages between nucleotides at positions 1 and 2, 2 and 3, 3 and 4, 20 and 21, and 21 and 22, numbered 5′ to 3′. In some aspects, the 3′ terminal overhang is 2 nucleotides. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

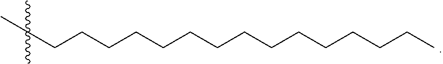 [SMILES: *CCCCCCCCCCCCCCC]

In some aspects, the lipid moiety is conjugated to the 2′ carbon of the ribose ring of the nucleotide. In some aspects, the lipid moiety is conjugated to a nucleotide at position 4 of the sense strand, numbered 5′ to 3′. In some aspects, S1 and S2 each comprise 1-6 nucleotides. In some aspects, wherein S1 and S2 are each 2 nucleotides. In some aspects, S1 and S2 each comprise one Tm-increasing nucleotide. In some aspects, the Tm-increasing nucleotide is a bicyclic nucleotide. In some aspects, Tm-increasing nucleotide is a locked nucleic acid (LNA). In some aspects, the remaining nucleotides comprise a 2′-O-methyl modification, provided that the nucleotide of the sense strand conjugated to the at least one lipid moiety does not comprise a 2′-O-methyl modification. In some aspects, L is 4 nucleotides. In some aspects, L comprises the sequence 5′-UNCG-3′, wherein N is any nucleotide. In some aspects, L comprises the sequence 5′-UACG-3. In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is a central nervous system (CNS) target sequence, optionally a neuronal mRNA target sequence or an ocular mRNA target sequence.

In some aspects, the disclosure provides a double-stranded oligonucleotide comprising an antisense strand of 22 nucleotides in length and a sense strand of 28 nucleotides in length, wherein the antisense and sense strands form an asymmetric duplex region of 22 base pairs comprising a 3′ terminal overhang of 2 nucleotides of the antisense strand, wherein the antisense strand comprises a region of complementarity to a central nervous system mRNA target sequence, wherein the sense strand comprises: (i) a stem-loop, wherein the stem-loop comprises a nucleotide sequence represented by the formula: 5′-S1-L-S2-3′, wherein S1 is complementary to S2, wherein S1 and S2 each comprise 2 nucleotides, wherein L forms a loop between S1 and S2 and comprises the sequence UNCG, and wherein S1 and S2 each comprise at least one Tm-increasing nucleotide, and (ii) at least one lipid moiety conjugated to a nucleotide of the loop, wherein the sense strand comprises an orientation of 5′ to 3′, wherein the stem-loop is at the 5′ end of the sense strand, wherein the sense strand comprises a 2′-fluoro modification at positions 18-21 and a 2′-O-methyl modification at positions 2-3, 5-7, 9-17 and 22-28, wherein the antisense strand comprises a 2′-fluoro modification at positions 2-5, 7, 10 and 14 and a 2-O-methyl modification at positions 1, 6, 8, 9, 11-13, and 15-22, wherein the sense strand comprises phosphorothioate linkages between positions 26 and 27, and 27 and 28, and wherein the antisense strand comprises phosphorothioate linkages between positions 1 and 2, 2 and 3, 3 and 4, 20 and 21, and 21 and 22.

In some aspects, the disclosure provides a double-stranded oligonucleotide comprising an antisense strand of about 20-22 nucleotides in length and a sense strand of about 32-34 nucleotides in length, wherein the antisense and sense strands form a duplex region of about 20-22 base pairs, and the oligonucleotide is blunt ended, wherein the antisense strand comprises a region of complementarity to a mRNA target sequence, wherein the sense strand comprises: (i) a stem-loop, wherein the stem-loop comprises a nucleotide sequence represented by the formula: 5′-S1-L-S2-3′, wherein S1 is complementary to S2, wherein L forms a loop between S1 and S2, and wherein S1 and S2 each comprise at least one Tm-increasing nucleotide, and (ii) at least one lipid moiety conjugated to a nucleotide of the loop, wherein the sense strand comprises an orientation of 5′ to 3′, wherein the stem-loop is at the 5′ end of the sense strand, and wherein each of the antisense and sense strands comprise at least one 2′-modified nucleotide and at least one modified internucleotide linkage.

In some aspects, the sense strand is 34 nucleotides and comprises a 2′-fluoro modification at positions 22-25, numbered 5′ to 3′. In some aspects, antisense strand is 22 nucleotides and comprises a 2′-fluoro modification at positions 2-5, 7, 10 and 14, numbered 5′ to 3′. In some aspects, the at least one modified internucleotide linkage is a phosphorothioate linkage. In some aspects, the sense strand is 34 nucleotides and comprises phosphorothioate linkages between nucleotides at positions 32 and 33, and 33 and 34, numbered 5′ to 3′. In some aspects, the antisense strand is 22 nucleotides and comprises phosphorothioate linkages between nucleotides at positions 1 and 2, 2 and 3, 3 and 4, 20 and 21, and 21 and 22, numbered 5′ to 3′. In some aspects, the oligonucleotide comprises a blunt end comprising the 5′ end of the antisense strand and the 3′ end of the sense strand. In some aspects, the antisense strand comprises a phosphorylated uridine at position 1, numbered 5′ to 3′. In some aspects, the phosphorylated uridine is 4′-O-monomethylphosphonae-2′-O-methyl uridine. In some aspects, the lipid moiety is a C16 hydrocarbon represented by:

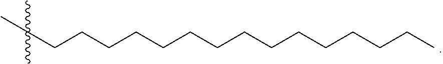 [SMILES: *CCCCCCCCCCCCCCC]

In some aspects, the lipid moiety is conjugated to the 2′ carbon of the ribose ring of the nucleotide. In some aspects, the lipid moiety is conjugated to a nucleotide at position 6 of the sense strand, numbered 5′ to 3′. In some aspects, S1 and S2 each comprise 1-6 nucleotides. In some aspects, S1 and S2 are each 4 nucleotides. In some aspects, S1 and S2 each comprise one Tm-increasing nucleotide. some aspects, the Tm-increasing nucleotide is a bicyclic nucleotide. In some aspects, the Tm-increasing nucleotide is a locked nucleic acid (LNA). In some aspects, the remaining nucleotides comprise a 2′-O-methyl modification, provided that the nucleotide of the sense strand conjugated to the at least one lipid moiety does not comprise a 2′-O-methyl modification. In some aspects, L is 4 nucleotides. In some aspects, L comprises the sequence 5′-GAAA-3′. In some aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence. In some aspects, the mRNA target sequence is (i) a central nervous system (CNS) mRNA target sequence, optionally a neuronal mRNA target sequence or an ocular mRNA target sequence; or (ii) an ocular tissue mRNA target sequence.

In some aspects, the disclosure provides a double-stranded oligonucleotide comprising an antisense strand of 22 nucleotides in length and a sense strand of 34 nucleotides in length, wherein the antisense and sense strands form a duplex region of 22 base pairs, and the oligonucleotide is blunt ended, wherein the antisense strand comprises a region of complementarity to a central nervous system mRNA target sequence, wherein the sense strand comprises: (i) a stem-loop, wherein the stem-loop comprises a nucleotide sequence represented by the formula: 5′-S1-L-S2-3′, wherein S1 is complementary to S2, wherein S1 and S2 each comprise 4 nucleotides, wherein L forms a loop between S1 and S2, wherein the loop comprises 4 nucleotides, and wherein S1 and S2 each comprise at least one Tm-increasing nucleotide, and (ii) at least one lipid moiety conjugated to a nucleotide of the loop, wherein the sense strand comprises an orientation of 5′ to 3′, wherein the stem-loop is at the 5′ end of the sense strand, wherein the sense strand comprises a 2′-fluoro modification at positions 22-25 and a 2′-O-methyl modification at positions 2-5, 7-11, 12-21 and 26-34, wherein the antisense strand comprises a 2′-fluoro modification at positions 2-5, 7, 10 and 14 and a 2-O-methyl modification at positions 1, 6, 8, 9, 11-13, and 15-22, wherein the sense strand comprises phosphorothioate linkages between positions 32 and 33, and 33 and 34, and wherein the antisense strand comprises phosphorothioate linkages between positions 1 and 2, 2 and 3, 3 and 4, 20 and 21, and 21 and 22.

In any of the foregoing or related aspects, the oligonucleotide is a Dicer substrate. In further aspects, the oligonucleotide reduces expression of the mRNA target sequence in a cell or population of cells in vitro and/or in vivo.

In some aspects, the disclosure provides a pharmaceutical composition comprising an oligonucleotide of any of the foregoing or related aspects, and a pharmaceutically acceptable carrier, delivery agent or excipient.

In some aspects, the disclosure provides a method for treating a subject having a disease, disorder or condition associated with expression of a target mRNA, the method comprising administering to the subject a therapeutically effective amount of an oligonucleotide or pharmaceutical composition described herein.

In some aspects of the methods described herein, the target mRNA is expressed in the central nervous system, optionally a neuron of the central nervous system. In some aspects of the methods described herein, the target mRNA is expressed in ocular tissue, optionally the optic nerve and/or retina.

In some aspects, the disclosure provides a method of delivering an oligonucleotide to a cell or population of cells in the central nervous system or ocular tissue, the method comprising administering a pharmaceutical composition described herein.

In some aspects, the disclosure provides a method of reducing expression of a target mRNA in a subject, comprising administering to the subject an oligonucleotide or pharmaceutical composition described herein.

In some aspects of the methods described herein, target mRNA is expressed in the central nervous system, optionally a neuron of the central nervous system. In some aspects of the methods described herein, the target mRNA is expressed in ocular tissue, optionally the optic nerve and/or retina.

In some aspects, the disclosure provides a kit comprising an oligonucleotide described herein, an optional pharmaceutically acceptable carrier, and a package insert comprising instructions for administration to a subject having a disease, disorder or condition associated with expression of a target mRNA.

In some aspects, the disclosure provides use of an oligonucleotide or pharmaceutical composition described herein, in the manufacture of a medicament for the treatment of a disease, disorder, or condition associated with expression of a target mRNA.

In some aspects, the disclosure provides any oligonucleotide or pharmaceutical composition described herein, for use, or adaptable for use, in the treatment of a disease, disorder, or condition associated with expression of a target mRNA.

In some aspects of the methods described herein, the target mRNA is expressed in the central nervous system and/or a neuron of the central nervous system. In some aspects, the target mRNA is expressed in ocular tissue, optionally the retina and/or optic nerve.

In any of the foregoing or related aspects of the methods described herein the central nervous system comprises the frontal cortex, the hippocampus, the cerebellum, the brainstem, lumbar dorsal root ganglion, the lumbar spinal cord, or combinations thereof.

In any of the foregoing or related aspects, the central nervous system comprises the frontal cortex, the hippocampus, the cerebellum, the brainstem, lumbar dorsal root ganglion, the lumbar spinal cord, or combinations thereof.

In some aspects, the disclosure provides a method of activating target-specific RNA interference (RNAi) in an organism comprising administering to said organism a dsRNA oligonucleotide described herein, said oligonucleotide being administered in an amount sufficient for degradation of the target mRNA to occur, thereby activating target-specific RNAi in the organism. In some aspects of, the target mRNA specifies the amino acid sequence of a protein involved or predicted to be involved in a human disease or disorder. In some aspects, the disease or disorder is selected from the group consisting of viral infections, bacterial infections, parasitic infections, cancers, allergies, autoimmune diseases, immunodeficiencies, and immunosuppression.

In some aspects, the disclosure provides a double-stranded oligonucleotide comprising an antisense strand of about 13-30 nucleotides in length and a sense strand of about 10-50 nucleotides in length, wherein the antisense and sense strands are separate strands which form an asymmetric duplex region having an overhang of about 2-10 nucleotides at the 3′ terminus of the antisense strand, wherein the duplex region is about 10-30 nucleotides, wherein the antisense strand comprises a region of complementarity to a mRNA target sequence, and wherein the sense strand comprises at least one lipid moiety conjugated to a nucleotide on the sense strand.

In some aspects, the disclosure provides a double-stranded oligonucleotide comprising an antisense strand of about 13-30 nucleotides in length and a sense strand of about 10-50 nucleotides in length, wherein the antisense and sense strands are separate strands which form an asymmetric duplex region having an overhang of about 2-12 nucleotides at the 3′ terminus of the antisense strand, wherein the duplex region is about 10-30 nucleotides, wherein the antisense strand comprises a region of complementarity to a mRNA target sequence, and wherein the sense strand comprises at least one lipid moiety conjugated to a nucleotide on the sense strand.

In some aspects, the oligonucleotide comprises a blunt end. In some aspects, the blunt end comprises the 3′ end of the sense strand and the 5′ end of the antisense strand.

In further aspects, the oligonucleotide comprises a stem loop, wherein the stem loop comprises a nucleotide sequence represented by the formula: 5′-S1-L-S2-3′, wherein S1 is complementary to S2, and wherein L forms a loop between S1 and S2, and wherein the sense strand comprises an orientation of 5′ to 3′, and wherein the stem-loop is at the 3′ end of the sense strand.

In any of the foregoing or related aspects,
(i) the sense strand is 19 nucleotides, the duplex region is 19 nucleotides, and the overhang is 3 nucleotides;
(ii) the sense strand is 18 nucleotides, the duplex region is 18 nucleotides, and the overhang is 4 nucleotides;
(iii) the sense strand is 17 nucleotides, the duplex region is 17 nucleotides, and the overhang is 5 nucleotides;
(iv) the sense strand is 16 nucleotides, the duplex region is 16 nucleotides, and the overhang is 6 nucleotides; or
(v) the sense strand is 15 nucleotides, the duplex region is 15 nucleotides, and the overhang is 7 nucleotides.

In some aspects,
(i) the sense strand is 19 nucleotides, the duplex region is 19 nucleotides, and the overhang is 3 nucleotides;
(ii) the sense strand is 18 nucleotides, the duplex region is 18 nucleotides, and the overhang is 4 nucleotides;
(iii) the sense strand is 17 nucleotides, the duplex region is 17 nucleotides, and the overhang is 5 nucleotides;
(iv) the sense strand is 16 nucleotides, the duplex region is 16 nucleotides, and the overhang is 6 nucleotides;
(v) the sense strand is 15 nucleotides, the duplex region is 15 nucleotides, and the overhang is 7 nucleotides;
(vi) the sense strand is 14 nucleotides, the duplex region is 14 nucleotides, and the overhang is 8 nucleotides;
(vii) the sense strand is 13 nucleotides, the duplex region is 13 nucleotides, and the overhang is 8 nucleotides; or
(viii) the sense strand is 12 nucleotides, the duplex region is 12 nucleotides, and the overhang is 10 nucleotides.

In some aspects, the sense strand is 10 nucleotides, the duplex region is 10 nucleotides, and the overhang is 12 nucleotides.

In some aspects,
(i) the sense strand is 32 nucleotides, the duplex region is 16 nucleotides, and the overhang is 6 nucleotides;
(ii) the sense strand is 30 nucleotides, the duplex region is 14 nucleotides, and the overhang is 8 nucleotides; or
(iii) the sense strand is 28 nucleotides, the duplex region is 12 nucleotides, and the overhang is 10 nucleotides.

In some aspects, the antisense strand is 22 nucleotides, with positions 1-22 numbered 5′ to 3′.

In some aspects, the lipid moiety is conjugated to a nucleotide of the sense strand that forms a base pair with a nucleotide at position 14 of the antisense strand. In some aspects, the lipid moiety is conjugated to a nucleotide of the sense strand that forms a base pair with a nucleotide at position 12 of the antisense strand. In some aspects, the lipid moiety is conjugated to a nucleotide of the sense strand that forms a base pair with a nucleotide at position 20, position 19, position 18, position 17, position 16, position 15, position 14, position 13, or position 12 of the antisense strand.

In further aspects, the lipid moiety is conjugated to a nucleotide in the loop. In some aspects, the lipid moiety is conjugated to the 3′ terminal nucleotide on the sense strand. In some aspects, the lipid moiety is conjugated to the 5′ terminal nucleotide on the sense strand.

In any of the foregoing or related aspects, the lipid moiety is selected from:

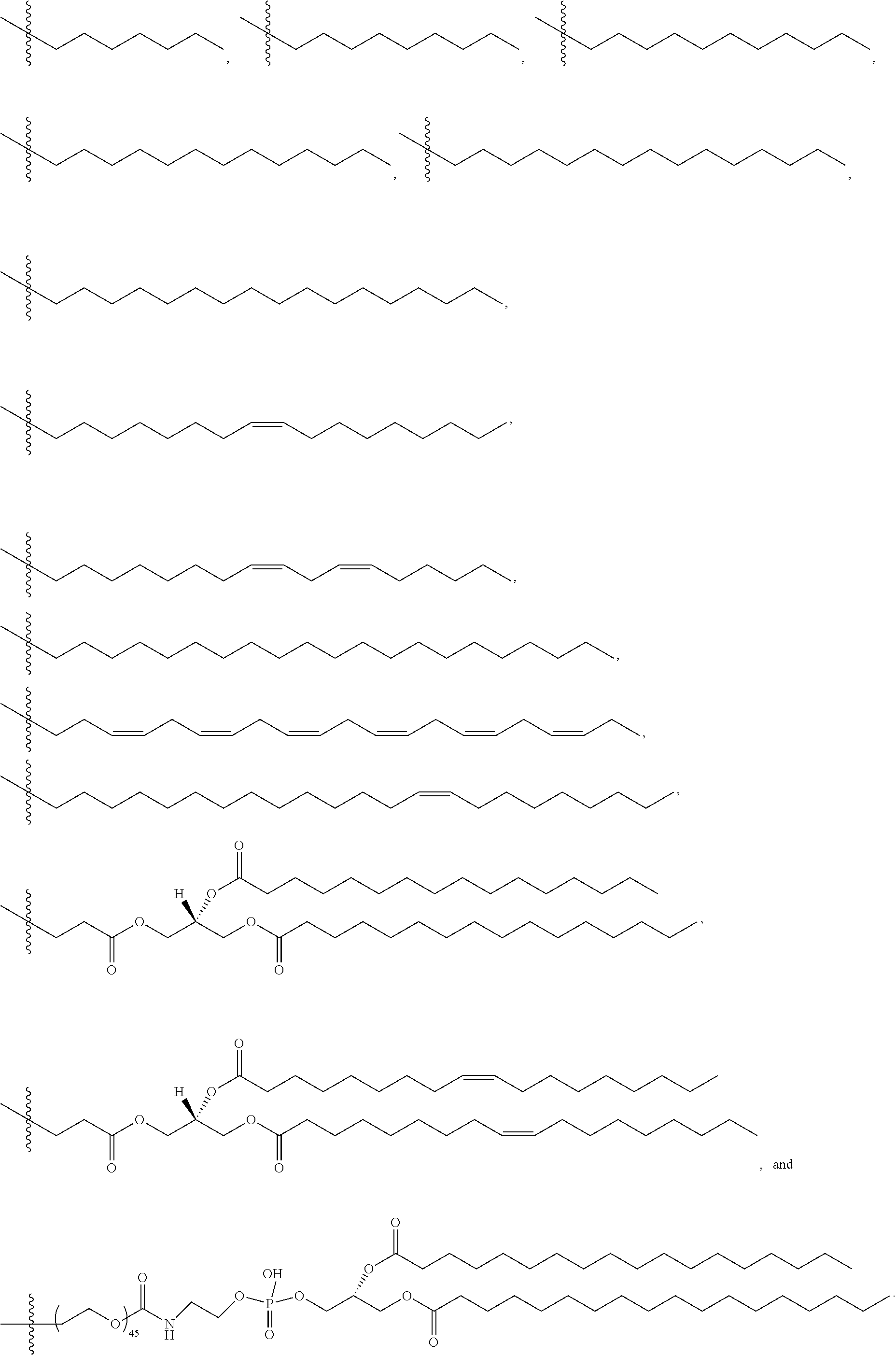

In some aspects, the lipid moiety is a hydrocarbon chain. In some aspects, the hydrocarbon chain is a C8-C30 hydrocarbon chain. In some aspects, the hydrocarbon chain is a C16 hydrocarbon chain. In some aspects, the C16 hydrocarbon chain is represented by

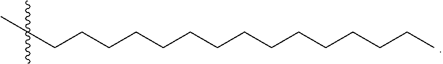 [SMILES: *CCCCCCCCCCCCCCC]

In some aspects, the hydrocarbon chain is a C22 hydrocarbon chain. In some aspects, the C22 hydrocarbon chain is represented by

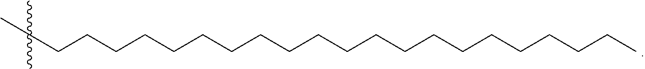 [SMILES: *CCCCCCCCCCCCCCCCCCCCC]

In any of the foregoing or related aspects, the lipid moiety is conjugated to the 2′ carbon of the ribose ring of the nucleotide.

In any of the foregoing or related aspects, the region of complementarity is fully complementary to the mRNA target sequence. In some aspects, the region of complementarity is partially complementary to the mRNA target sequence. In some aspects, the region of complementarity comprises no more than four mismatches to the mRNA target sequence.

In any of the foregoing or related aspects, the mRNA target sequence is a liver mRNA target sequence, optionally a liver macrophage mRNA target sequence, a liver hepatocyte mRNA target sequence, or a liver sinusoidal endothelial cell mRNA target sequence. In some aspects, the mRNA target sequence is an ocular mRNA target sequence. In some aspects, the mRNA target sequence is expressed in liver tissue, skeletal muscle tissue, adipose tissue and/or adrenal tissue. In other aspects, the mRNA target sequence is expressed in at least one tissue of the central nervous system. In some aspects, the at least one tissue of the central nervous system is selected from frontal cortex, medulla, hippocampus, hypothalamus, cerebellum, lumbar spinal cord, lumbar dorsal root ganglion, and any combination thereof.

In any of the foregoing or related aspects, oligonucleotide comprises at least one modified nucleotide. In some aspects, the modified nucleotide comprises a 2′-modification. In some aspects, each of the nucleotides of the sense strand and the antisense strand comprise a 2′-modification. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, the antisense strand is 22 nucleotides with positions 1-22 numbered 5′ to 3′, and wherein the sense strand comprises a 2′-fluoro modification at each of nucleotides forming a base pair with nucleotides at positions 10-13 of the antisense strand. In some aspects, the antisense strand is 22 nucleotides with positions 1-22 numbered 5′ to 3′, and wherein the sense strand comprises a 2′-fluoro modification at each of nucleotides forming a base pair with nucleotides at positions 10, 11, 12, 13, or any combination thereof, of the antisense strand. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects, the remaining nucleotides comprise a 2′-O-methyl modification, provided that the nucleotide of the sense strand conjugated to the at least one lipid moiety does not comprise a 2′-O-methyl modification.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the antisense strand comprises a phosphorothioate linkage between positions 13 and 14 and between positions 14 and 15. In some aspects, the antisense strand comprises a phosphorothioate linkage between positions 16 and 17, between positions 17 and 18, between positions 18 and 19, and between positions 19 and 20. In some aspects, the antisense strand comprises a phosphorothioate linkage between positions 15 and 16, between positions 16 and 17, between positions 17 and 18, between positions 18 and 19, and between positions 19 and 20. In some aspects, the antisense strand comprises a phosphorothioate linkage between positions 12 and 13, between positions 15 and 16, between positions 16 and 17, between positions 17 and 18, between positions 18 and 19, and between positions 19 and 20. In some aspects, the sense strand comprises a phosphorothioate linkage between positions 1 and 2. In some aspects, the sense strand comprises a phosphorothioate linkage between positions 1 and 2, between the penultimate nucleotide and third nucleotide from the 3′ end, and between the penultimate nucleotide and ultimate nucleotide.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate. In some aspects, the phosphorylated nucleotide is 4′-O-monomethylphosphonate-2′-O-methyl uridine.

In any of the foregoing or related aspects, the sense strand comprises at least one Tm-increasing nucleotide. In some aspects, the sense strand comprises up to nine Tm-increasing nucleotides. In some aspects, the sense strand comprises 1-3 Tm-increasing nucleotides. In some aspects, S1 and S2 each comprise at least one Tm-increasing nucleotide. In some aspects, S1 and S2 are each 3 nucleotides, and each nucleotide is a Tm-increasing nucleotide. In some aspects, the sense strand comprises up to three Tm-increasing nucleotides at nucleotide positions, provided the nucleotide positions are not in the stem-loop. In some aspects, wherein the sense strand comprises up to five Tm-increasing nucleotides.

In any of the foregoing or related aspects, the Tm-increasing nucleotide is a bicyclic nucleotide. In some aspects, the Tm-increasing nucleotide is a locked nucleic acid. In some aspects, the sense strand is 20 nucleotides in length, wherein the nucleotides are numbered 1-20 5′ to 3′, and wherein the sense strand comprises a locked nucleic acid at a nucleotide located at:
(i) position 2;
(ii) position 2 and position 15;
(iii) position 2, position 15, and position 16;
(iv) position 2, position 15, position 16, and position 18; or,
(v) position 2, position 15, position 16, position 18, and position 19.

In some aspects, the sense strand is 16 nucleotides in length, wherein the nucleotides are numbered 1-16 5′ to 3′, and wherein the sense strand comprises a locked nucleic acid at a nucleotide located at:
(i) position 2;
(ii) position 2 and position 11;
(iii) position 2, position 11, and position 12;
(iv) position 2, position 11, position 12, and position 14; or,
(v) position 2, position 11, position 12, position 14, and position 15.

In some aspects, the sense strand is 14 nucleotides in length, wherein the nucleotides are numbered 1-14 5′ to 3, and wherein the sense strand comprises a locked nucleic acid at a nucleotide located at:
(i) position 2;
(ii) position 2 and position 9;
(iii) position 2, position 9, and position 10; or,
(iv) position 2, position 9, position 10, position 12, and position 13.

In some aspects, the sense strand is 12 nucleotides in length, wherein the nucleotides are numbered 1-12 5′ to 3′, and wherein the sense strand comprises a locked nucleic acid at a nucleotide located at:
(i) position 2;
(ii) position 2 and position 7;
(iii) position 2, position 7, and position 8;
(iv) position 2, position 7, position 8, and position 10;
(v) position 2, position 7, position 8, position 10, and position 11.

In some aspects, the sense strand comprises a locked nucleic acid at a nucleotide that forms a base pair with a nucleotide at a position of the antisense strand selected from:
(i) position 2;
(ii) position 3;
(iii) position 5;
(iv) position 6; and
(v) any combination of (i)-(iv).

In any of the foregoing or related aspects, the oligonucleotide is a Dicer substrate. In some aspects, the oligonucleotide reduces expression of the mRNA target sequence in a cell or population of cells in vitro and/or in vivo.

In some aspects, the disclosure provides a pharmaceutical composition comprising any oligonucleotide described herein, and a pharmaceutically acceptable carrier, delivery agent or excipient.

In some aspects, the disclosure provides a method of delivering an oligonucleotide to a cell or population of cells in the central nervous system, liver tissue, muscle tissue, adipose tissue, adrenal tissue and/or ocular tissue, the method comprising administering a pharmaceutical composition described herein.

In some aspects, the disclosure provides a method for treating a subject having a disease, disorder or condition associated with expression of a target mRNA, the method comprising administering to the subject a therapeutically effective amount of an oligonucleotide or pharmaceutical composition described herein.

In some aspects, the disclosure provides a method of reducing expression of a target mRNA in a subject, comprising administering to the subject an oligonucleotide or pharmaceutical composition described herein.

In some aspects of the methods described herein, the target mRNA is expressed in the liver. In some aspects, the target mRNA is expressed in a hepatocyte. In some aspects, the target mRNA is expressed in a liver sinusoidal endothelial cell. In some aspects of the methods described herein, the target mRNA is expressed in a macrophage of the liver. In some aspects, the target mRNA is expressed in at least one cell type of the central nervous system. In some aspects, the at least one cell type of the central nervous system is an astrocyte, a neuron, or an oligodendrocyte. In some aspects, the target mRNA is expressed in an astrocyte, a neuron, an oligodendrocyte, or any combination thereof. In some aspects, the target mRNA is expressed in the frontal cortex, medulla, hippocampus, hypothalamus, cerebellum, lumbar spinal cord, lumbar dorsal root ganglion, or any combination thereof. In some aspects of the methods described herein, the target mRNA is expressed in ocular tissue, optionally the retina or optic nerve. In some aspects of the methods described herein, the target mRNA is expressed in liver tissue, skeletal muscle tissue, adipose tissue and/or adrenal tissue.

In some aspects, the disclosure provides a kit comprising an oligonucleotide described herein, an optional pharmaceutically acceptable carrier, and a package insert comprising instructions for administration to a subject having a disease, disorder or condition associated with expression of a target mRNA.

In some aspects, the disclosure provides use of an oligonucleotide or pharmaceutical composition described herein in the manufacture of a medicament for the treatment of a disease, disorder, or condition associated with expression of a target mRNA.

In some aspects, the disclosure provides an oligonucleotide or pharmaceutical composition described herein, for use, or adaptable for use, in the treatment of a disease, disorder, or condition associated with expression of a target mRNA.

In any of the foregoing or related aspects, the target mRNA is expressed in the liver, optionally wherein the target mRNA is expressed in a liver macrophage, a liver hepatocyte, or a liver sinusoidal endothelial cell. In any of the foregoing or related aspects, the target mRNA is expressed in ocular tissue, optionally the retina or optic nerve. In any of the foregoing or related aspects, the target mRNA is expressed in liver tissue, skeletal muscle tissue, adipose tissue and/or adrenal tissue. In some aspects, the target mRNA is expressed in at least one tissue of the central nervous system. In some aspects, the at least one tissue of the central nervous system is selected from the frontal cortex, medulla, hippocampus, hypothalamus, cerebellum, lumbar spinal cord, lumbar dorsal root ganglion, and any combinations thereof.

In any of the foregoing or related aspects of the methods described herein, the disease or disorder is selected from the group consisting of viral infections, bacterial infections, parasitic infections, cancers, allergies, autoimmune diseases, immunodeficiencies, and immunosuppression.

In some aspects, the disclosure provides a double-stranded oligonucleotide comprising:
(i) a sense strand 14-20 nucleotides in length, wherein the sense strand comprises at least one lipid moiety conjugated to the 5′ terminus, and wherein the sense strand comprises at least one locked nucleic acid, and
(ii) an antisense strand 22 nucleotides in length, wherein the antisense strand comprises a region of complementarity to an astrocyte target mRNA, and wherein the sense and antisense strand are separate strands which form an asymmetric duplex region having an overhang of about 2-8 nucleotides at the 3′ terminus of the antisense strand, wherein the duplex region is about 14-20 nucleotides.

In other aspects, the disclosure provides a double-stranded oligonucleotide comprising:
(i) a sense strand 14-20 nucleotides in length, wherein the sense strand comprises at least one lipid moiety conjugated to the 5′ terminus, and wherein the sense strand comprises at least one locked nucleic acid, and
(ii) an antisense strand 22 nucleotides in length, wherein the antisense strand comprises a region of complementarity to a neuron target mRNA, and
wherein the sense and antisense strand are separate strands which form an asymmetric duplex region having an overhang of about 2-8 nucleotides at the 3′ terminus of the antisense strand, wherein the duplex region is about 14-20 nucleotides.

In further aspects, the disclosure provides a double-stranded oligonucleotide comprising:
(i) a sense strand 14-20 nucleotides in length, wherein the sense strand comprises at least one lipid moiety conjugated to the 5′ terminus, and wherein the sense strand comprises at least one locked nucleic acid, and
(ii) an antisense strand 22 nucleotides in length, wherein the antisense strand comprises a region of complementarity to an oligodendrocyte target mRNA, and
wherein the sense and antisense strand are separate strands which form an asymmetric duplex region having an overhang of about 2-8 nucleotides at the 3′ terminus of the antisense strand, wherein the duplex region is about 14-20 nucleotides.

In any of the foregoing or related aspects, the sense strand is 14 nucleotides in length. In some aspects, the sense strand comprises a locked nucleic acid at one or more of position 2, position 9, position 10, position 12, or position 13 with positions numbered 5′ to 3′. In some aspects, the antisense strand comprises a phosphorothioate linkage between positions 1 and 2, between positions 2 and 3, between positions 3 and 4, between positions 13 and 14, between positions 14 and 15, between positions 20 and 21, and between positions 21 and 22.

In any of the foregoing or related aspects, the sense strand is 20 nucleotides in length. In some aspects, the sense strand comprises a locked nucleic acid at one or more of position 2, position 15, or position 16 with positions numbered 5′ to 3′. In some aspects, the antisense strand comprises a phosphorothioate linkage between positions 1 and 2, between positions 2 and 3, between positions 3 and 4, between positions 20 and 21, and between positions 21 and 22.

In other aspects, the disclosure provides a method of reducing expression of a target mRNA in an astrocyte, comprising administering a double-stranded oligonucleotide wherein the oligonucleotide comprises:
(i) a sense strand, wherein the sense strand is 14-20 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to a target mRNA in the astrocyte, and
wherein the sense and antisense strand are separate strands which form an asymmetric duplex region having an overhang of about 2-8 nucleotides at the 3′ terminus of the antisense strand, wherein the duplex region is about 14-20 nucleotides, thereby reducing expression of the target mRNA in the astrocyte. In some aspects, the oligonucleotide comprises a blunt end comprising the 3′ end of the sense strand and the 5′ end of the antisense strand. In some aspects, the sense strand comprises no more than 3 locked nucleic acids. In some aspects, the sense strand is 20 nucleotides in length, and wherein reduction of the target mRNA in an astrocyte is increased compared to reduction in a neuron. In some aspects, reduction of the target mRNA is increased by at least 5%. In some aspects, reduction of the target mRNA is increased by at least 5%, by at least 10%, by at least 15%, by at least 20%, by at least 25%, by at least 30%, by at least 35%, by at least 40%, by at least 45% or by at least 50%. In some aspects, the sense strand is 14 nucleotides in length, and wherein reduction of the target mRNA in an astrocyte is increased compared to reduction in an oligodendrocyte. In some aspects, reduction of the target mRNA is increased by at least 5%. In some aspects, reduction of the target mRNA is increased by at least 5%, by at least 10%, by at least 15%, by at least 20%, by at least 25%, by at least 30%, by at least 35%, by at least 40%, by at least 45% or by at least 50%. In some aspects, the sense strand is 20 nucleotides in length, and wherein the target mRNA is reduced in an astrocyte and an oligodendrocyte to the same or similar level. In some aspects, the sense strand is 14 nucleotides in length, wherein the target mRNA is reduced in an astrocyte and in a neuron to the same or similar level, and wherein reduction of the target mRNA in an astrocyte and in a neuron is increased compared to reduction in an oligodendrocyte. In some aspects, reduction of the target mRNA is increased by at least 5%. In some aspects, reduction of the target mRNA is increased by at least 5%, by at least 10%, by at least 15%, by at least 20%, by at least 25%, by at least 30%, by at least 35%, by at least 40%, by at least 45% or by at least 50%.

In other aspects, the disclosure provides a method of reducing expression of a target mRNA in an oligodendrocyte, comprising administering a double-stranded oligonucleotide wherein the oligonucleotide comprises:
(i) a sense strand, wherein the sense strand is 14-40 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to a target mRNA in the oligodendrocyte, and
wherein the sense and antisense strand are separate strands which form an asymmetric duplex region having an overhang of about 2-8 nucleotides at the 3′ terminus of the antisense strand, wherein the duplex region is about 14-20 nucleotides, thereby reducing expression of the target mRNA in the oligodendrocyte. In some aspects, the oligonucleotide comprises a blunt end comprising the 3′ end of the sense strand and the 5′ end of the antisense strand. In some aspects, the sense strand is 20 nucleotides in length. In some aspects, the sense strand in 36 nucleotides in length, and wherein the oligonucleotide comprises a stem-loop. In some aspects, the target mRNA is reduced in an astrocyte and the oligodendrocyte to the same or similar level, and wherein reduction of the target mRNA is increased in the astrocyte and the oligodendrocyte compared to reduction in a neuron. In some aspects, reduction of the target mRNA is increased by at least 5%. In some aspects, reduction of the target mRNA is increased by at least 5%, by at least 10%, by at least 15%, by at least 20%, by at least 25%, by at least 30%, by at least 35%, by at least 40%, by at least 45% or by at least 50%.

In other aspects, the disclosure provides a method of reducing expression of a target mRNA in a neuron, comprising administering a double-stranded oligonucleotide wherein the oligonucleotide comprises:
(i) a sense strand, wherein the sense strand is 14-20 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to a target mRNA in the neuron, and
wherein the sense and antisense strand are separate strands which form an asymmetric duplex region having an overhang of about 2-8 nucleotides at the 3′ terminus of the antisense strand, wherein the duplex region is about 14-20 nucleotides, thereby reducing expression of the target mRNA in the neuron. In some aspects, the oligonucleotide comprises a blunt end comprising the 3′ end of the sense strand and the 5′ end of the antisense strand. In some aspects, the sense strand comprises no more than 5 locked nucleic acids. In some aspects, the sense strand is 14 nucleotides in length.

The mammalian CNS is a complex system of tissues, including cells, fluids and chemicals that interact in concert to enable a wide variety of functions, including movement, navigation, cognition, speech, vision, and emotion. Unfortunately, a variety of diseases and disorders of the CNS are known (e.g., neurological disorders) and affect or disrupt some or all of these functions. Typically, treatments for diseases and disorders of the CNS have been limited to small molecule drugs, antibodies and/or to adaptive or behavioral therapies. There exists an ongoing need to develop treatment of diseases and disorders of the CNS associated with inappropriate gene expression.

The present disclosure is based, at least in part, on the discovery of lipid-conjugated RNAi oligonucleotides that effectively reduce target gene expression in astrocytes of the CNS. Exemplary lipid-conjugated RNAi oligonucleotides provided herein have demonstrated reduction of target gene expression of astrocyte-specific mRNA in the CNS following a single administration. Further, exemplary lipid-conjugated RNAi oligonucleotides provided herein have demonstrated pharmacological activity in multiple regions throughout the CNS, including difficult to reach areas such as the hippocampus and frontal cortex. Without being bound by theory, the hydrophobic moiety (e.g., lipid) facilitates delivery and distribution of the lipid-conjugated RNAi oligonucleotides into the CNS, thereby increasing efficacy and durability of gene knockdown in astrocytes. Accordingly, the disclosure provides methods of treating a disease or disorder by modulating expression of an astrocyte gene in the CNS using the lipid-conjugated RNAi oligonucleotides, and pharmaceutically acceptable compositions thereof, described herein. The disclosure further provides methods of using the lipid-conjugated RNAi oligonucleotides in the manufacture of a medicament for treating a disease or disorder by modulating expression of an astrocyte gene in the CNS.

Accordingly, in some aspects the disclosure provides a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to an astrocyte mRNA target sequence, and wherein the sense strand comprises at least one lipid moiety conjugated to a nucleotide of the sense strand.

In some aspects, the lipid moiety is selected from

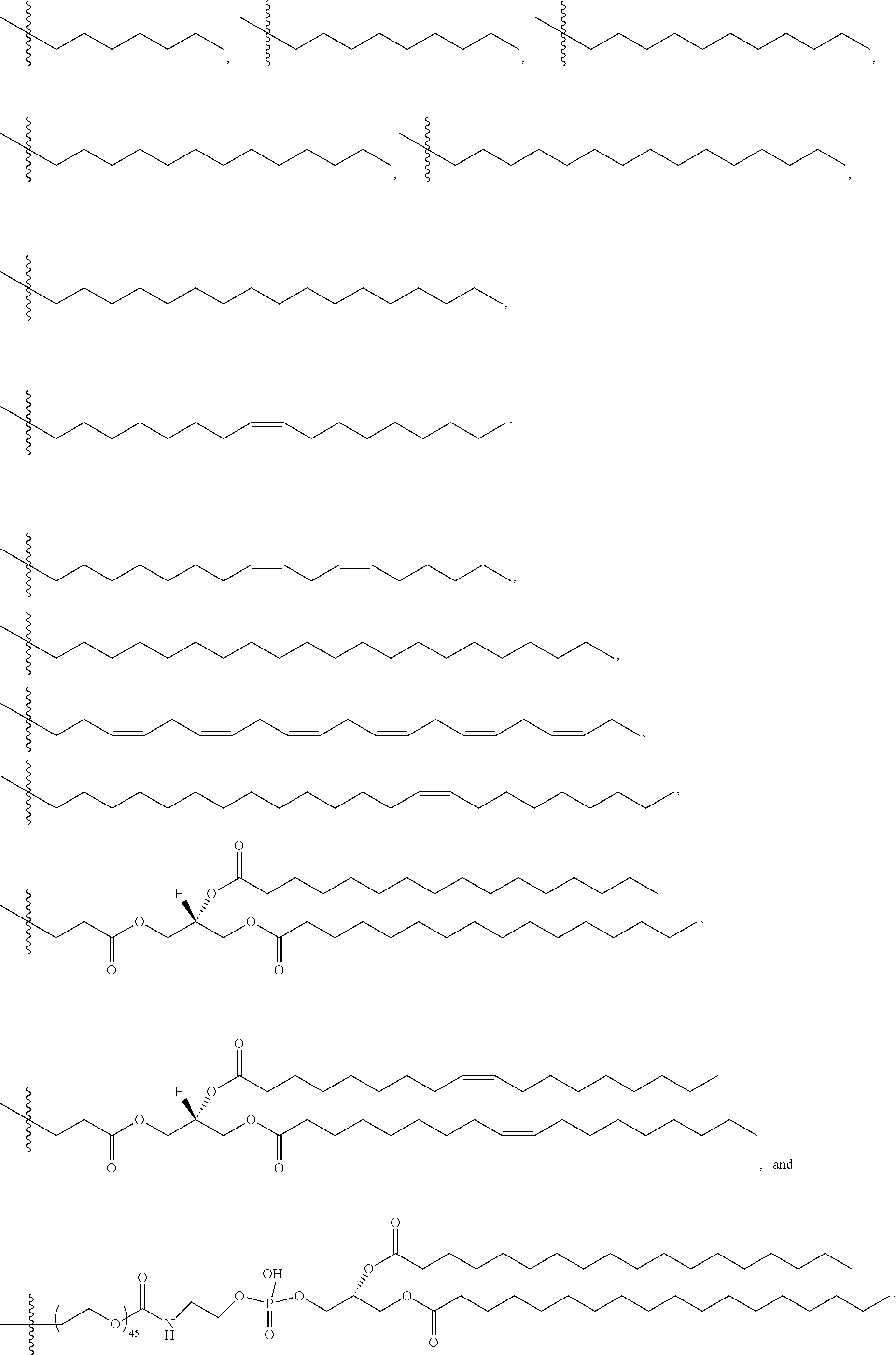

In some aspects, the lipid moiety is a hydrocarbon chain. In some aspects, the hydrocarbon chain is a C8-C30 hydrocarbon chain. In some aspects, the hydrocarbon chain is a C16 hydrocarbon chain. In some aspects, the C16 hydrocarbon chain is represented by

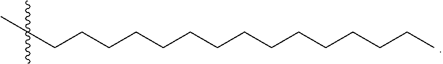 [SMILES: *CCCCCCCCCCCCCCC]

In any of the foregoing or related aspects, the lipid moiety is conjugated to the 2′ carbon of the ribose ring of the nucleotide.

In any of the foregoing or related aspects, the oligonucleotide is blunt ended. In some aspects, the oligonucleotide is blunt ended at the 3′ terminus of the oligonucleotide. In some aspects, the oligonucleotide comprises a blunt end. In some aspects, the blunt end comprises the 3′ terminus of the sense strand. In some aspects, the sense strand is 20-22 nucleotides. In some aspects, the at least one lipid moiety is conjugated to a nucleotide at position 1, position 4, position 8, position 12, position 13, position 18, or position 20 of the sense strand, wherein positions are numbered 5′ to 3′. In some aspects, the astrocyte mRNA target is expressed in the spinal cord, wherein the at least one lipid moiety is conjugated to a nucleotide at position 1, position 4, position 8, position 12, position 13, position 18 or position 20 of the sense strand, and wherein positions are numbered 5′ to 3′. In other aspects, the astrocyte mRNA target is expressed in the medulla, wherein the at least one lipid moiety is conjugated to a nucleotide at position 1, position 4, position 8, position 12, position 13, position 18 or position 20 of the sense strand, and wherein positions are numbered 5′ to 3′. In further aspects, the astrocyte mRNA target is expressed in the cerebellum, wherein the at least one lipid moiety is conjugated to a nucleotide at position 4, position 12, position 13, position 18 or position 20 of the sense strand, and wherein positions are numbered 5′ to 3′. In some aspects, the astrocyte mRNA target is expressed in the hypothalamus, wherein the at least one lipid moiety is conjugated to a nucleotide at position 1, position 4, position 12, position 13, position 18 or position 20 of the sense strand, and wherein positions are numbered 5′ to 3′. In other aspects, the astrocyte mRNA target is expressed in the frontal cortex, wherein the at least one lipid moiety is conjugated to a nucleotide at position 4 of the sense strand, and wherein positions are numbered 5′ to 3′.

In any of the foregoing or related aspects, the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2. In some aspects, the sense strand is 36-38 nucleotides. In some aspects, the astrocyte mRNA target is expressed in the spinal cord, wherein the at least one lipid moiety is conjugated to a nucleotide at position 1, position 4, position 8, position 12, position 13, position 18, position 20, position 23, position 28, position 29 or position 30 of the sense strand, and wherein positions are numbered 5′ to 3′. In other aspects, the astrocyte mRNA target is expressed in the medulla, wherein the at least one lipid moiety is conjugated to a nucleotide at position 1, position 4, position 18, position 20, position 23, position 28, position 29 or position 30 of the sense strand, and wherein positions are numbered 5′ to 3′. In further aspects, the astrocyte mRNA target is expressed in the cerebellum, wherein the at least one lipid moiety is conjugated to a nucleotide at position 1, position 4, position 23, position 28, or position 29 of the sense strand, and wherein positions are numbered 5′ to 3′. In some aspects, the astrocyte mRNA target is expressed in the hypothalamus, wherein the at least one lipid moiety is conjugated to a nucleotide at position 1, position 4, position 12, position 13, position 18, position 20, position 23, position 28, position 29 or position 30 of the sense strand, and wherein positions are numbered 5′ to 3′. In other aspects, the astrocyte mRNA target is expressed in the frontal cortex, wherein the at least one lipid moiety is conjugated to a nucleotide at position 23 of the sense strand, and wherein positions are numbered 5′ to 3′.

In any of the foregoing or related aspects, the antisense strand is 22-24 nucleotides. In some aspects, the duplex region is 20-22 base pairs.

In any of the foregoing or related aspects, the antisense strand comprises a 1-4 nucleotide overhang at the 3′ terminus. In some aspects, the overhang comprises purine nucleotides. In some aspects, the overhang sequence is 2 nucleotides in length. In some aspects, the overhang is selected from AA, GG, AG, and GA. In some aspects, the overhang is GG or AA. In some aspects, the overhang is GG.

In any of the foregoing or related aspects, the region of complementarity is complementary to at least 15 consecutive nucleotides of the astrocyte mRNA target sequence. In some aspects, the region of complementarity is complementary to 19 consecutive nucleotides of the astrocyte mRNA target sequence. In some aspects, the region of complementarity is fully complementary to the astrocyte mRNA target sequence. In some aspects, the region of complementarity is partially complementary to the astrocyte mRNA target sequence. In some aspects, the region of complementarity comprises no more than four mismatches to the astrocyte mRNA target sequence. In some aspects, the region of complementarity comprises up to four mismatches to the astrocyte mRNA target sequence.

In any of the foregoing or related aspects, the oligonucleotide comprises at least one modified nucleotide. In some aspects, the modified nucleotide comprises a 2′-modification. In some aspects, each of the nucleotides of the sense strand and the antisense strand comprise a 2′-modification except the nucleotide of the sense strand conjugated to the at least one lipid moiety. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, about 10-20%, 10%, 11%, 12%, 13%, 14% 15%, 16%, 17%, 18%, 19% or 20% of the nucleotides of the sense strand comprise a 2′-fluoro modification. In some aspects, about 25-35%, 25%, 26%, 27%, 28%, 29%, 30%, 31%, 32%, 33%, 34% or 35% of the nucleotides of the antisense strand comprise a 2′-fluoro modification. In some aspects, about 25-35%, 25%, 26%, 27%, 28%, 29%, 30%, 31%, 32%, 33%, 34% or 35% of the nucleotides of the oligonucleotide comprise a 2′-fluoro modification. In some aspects, the sense strand comprises 20 nucleotides with positions 1-20 from 5′ to 3′, wherein each of positions 8-11 comprise a 2′-fluoro modification. In some aspects, the sense strand comprises 20 nucleotides with positions 1-20 from 5′ to 3′, wherein each of positions 9-11 comprise a 2′-fluoro modification. In some aspects, the sense strand comprises 36 nucleotides with positions 1-36 from 5′ to 3′, wherein each of positions 8-11 comprise a 2′-fluoro modification. In some aspects, the sense strand comprises 36 nucleotides with positions 1-36 from 5′ to 3′, wherein each of positions 9-11 comprise a 2′-fluoro modification. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects, the remaining nucleotides comprise a 2′-O-methyl modification except the nucleotide of the sense strand conjugated to the at least one lipid moiety.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the sense strand comprises a phosphorothioate linkage between position 1 and 2, wherein positions are numbered 1-2 from 5′ to 3′. In some aspects, the sense strand is 20 nucleotides in length, and wherein the sense strand comprises a phosphorothioate linkage between positions 18 and 19, and between positions 19 and 20, wherein positions are numbered 1-22 from 5′ to 3′.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate.

In any of the foregoing or related aspects, the region of complementary is fully complementary to the astrocyte mRNA target sequence at nucleotide positions 2-8 of the antisense strand, wherein nucleotide positions are numbered 5′ to 3′. In some aspects, the region of complementary is fully complementary to the astrocyte mRNA target sequence at nucleotide positions 2-11 of the antisense strand, wherein nucleotide positions are numbered 5′ to 3′.

In any of the foregoing or related aspects, the oligonucleotide is a Dicer substrate. In some aspects, the oligonucleotide is a Dicer substrate that, upon endogenous Dicer processing, yields double-stranded nucleic acids of 19-21 nucleotides in length capable of reducing an astrocyte mRNA expression in a mammalian cell.

In any of the foregoing or related aspects, the astrocyte mRNA target sequence is located in a region of the central nervous system (CNS). In some aspects, the region of the CNS is selected from the spinal cord, lumbar spinal cord, thoracic spinal cord, cervical spinal cord, medulla, hippocampus, cerebellum, hypothalamus, frontal cortex, and a combination thereof.

In any of the foregoing or related aspects, the oligonucleotide reduces expression of a target mRNA in an astrocyte or population of astrocytes in vitro and/or in vivo.

In some aspects, the disclosure provides a pharmaceutical composition comprising an oligonucleotide described herein, and a pharmaceutically acceptable carrier, delivery agent or excipient.

In other aspects, the disclosure provides a method for treating a subject having a disease, disorder or condition associated with expression of an astrocyte mRNA, the method comprising administering to the subject a therapeutically effective amount of an oligonucleotide or pharmaceutical composition described herein, thereby treating the subject.

In further aspects, the disclosure provides a method of delivering an oligonucleotide to an astrocyte or a population of astrocytes in a subject, the method comprising administering a pharmaceutical composition described herein to the subject. In some aspects, the astrocyte or a population of astrocytes is located in a region of the CNS. In some aspects, the region of the CNS is selected from the spinal cord, lumbar spinal cord, thoracic spinal cord, cervical spinal cord, medulla, hippocampus, cerebellum, hypothalamus, frontal cortex, and a combination thereof.

In other aspects, the disclosure provides a method for reducing expression of an astrocyte mRNA in a cell, a population of cells or a subject, the method comprising the step of:
i. contacting the cell or the population of cells with an oligonucleotide or pharmaceutical composition described herein, optionally wherein the cell or population of cells is an astrocyte or a population of astrocytes; or
ii. administering to the subject an oligonucleotide or pharmaceutical composition described herein. In some aspects, reducing expression of the astrocyte mRNA comprises reducing an amount or level of mRNA, an amount or level of protein, or both. In some aspects, the subject has a disease, disorder or condition associated with expression of the astrocyte mRNA. In some aspects, the cell or population of cells is located in a region of the CNS. In some aspects, the region of the CNS is selected from the spinal cord, lumbar spinal cord, thoracic spinal cord, cervical spinal cord, medulla, hippocampus, cerebellum, hypothalamus, frontal cortex, and a combination thereof.

In any of the foregoing or related aspects, the methods comprise administering via intrathecal administration.

In some aspects, the disclosure provides a method of reducing expression of a target mRNA expressed in an astrocyte in a tissue of the CNS of a subject, comprising administering to the subject a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a target sequence in the target mRNA, and wherein the sense strand comprises at least one lipid moiety conjugated to a nucleotide of the sense strand. In some aspects, the lipid moiety is a C16 hydrocarbon.

In any of the foregoing or related aspects of the methods described herein, the oligonucleotide is blunt ended at the 3′ terminus of the oligonucleotide. In some aspects, the oligonucleotide comprises a blunt end. In some aspects, the blunt end comprises the 3′ terminus of the sense strand. In some aspects, the sense strand is 22-24 nucleotides. In some aspects, the tissue is the spinal cord, wherein the at least one lipid moiety is conjugated to a nucleotide at position 1, position 4, position 8, position 12, position 13, position 18 or position 20 of the sense strand, and wherein positions are numbered 5′ to 3′. In other aspects, the tissue is the medulla, wherein the at least one lipid moiety is conjugated to a nucleotide at position 1, position 4, position 8, position 12, position 13, position 18 or position 20 of the sense strand, and wherein positions are numbered 5′ to 3′. In further aspects, the tissue is the cerebellum, wherein the at least one lipid moiety is conjugated to a nucleotide at position 4, position 12, position 13, position 18 or position 20 of the sense strand, and wherein positions are numbered 5′ to 3′. In some aspects, the tissue is the hypothalamus, wherein the at least one lipid moiety is conjugated to a nucleotide at position 1, position 4, position 12, position 13, position 18 or position 20 of the sense strand, and wherein positions are numbered 5′ to 3. In other aspects, the tissue is the frontal cortex, wherein the at least one lipid moiety is conjugated to a nucleotide at position 4 of the sense strand, and wherein positions are numbered 5′ to 3′.

In any of the foregoing or related aspects of the methods described herein, the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2. In some aspects, the sense strand is 36-38 nucleotides. In some aspects, the tissue is the spinal cord, wherein the at least one lipid moiety is conjugated to a nucleotide at position 1, position 4, position 8, position 12, position 13, position 18, position 20, position 23, position 28, position 29 or position 30 of the sense strand, and wherein positions are numbered 5′ to 3′. In other aspects, the tissue is the medulla, wherein the at least one lipid moiety is conjugated to a nucleotide at position 1, position 4, position 18, position 20, position 23, position 28, position 29 or position 30 of the sense strand, and wherein positions are numbered 5′ to 3′. In further aspects, the tissue is the cerebellum, wherein the at least one lipid moiety is conjugated to a nucleotide at position 1, position 4, position 23, position 28, or position 29 of the sense strand, and wherein positions are numbered 5′ to 3′. In some aspects, the tissue is the hypothalamus, wherein the at least one lipid moiety is conjugated to a nucleotide at position 1, position 4, position 12, position 13, position 18, position 20, position 23, position 28, position 29 or position 30 of the sense strand, and wherein positions are numbered 5′ to 3′. In other aspects, the tissue is the frontal cortex, wherein the at least one lipid moiety is conjugated to a nucleotide at position 23 of the sense strand, and wherein positions are numbered 5′ to 3′.

In any of the foregoing or related aspects of the methods described herein, a single dose of the oligonucleotide or pharmaceutical composition reduces expression of the astrocyte mRNA for at least 4 weeks, at least 8 weeks, at least 12 weeks, at least 23 weeks, at least 26 weeks, or at least 29 weeks. In some aspects, a single dose of the oligonucleotide or pharmaceutical composition reduces expression of the astrocyte mRNA for at least 4 weeks. In some aspects, a single dose of the oligonucleotide or pharmaceutical composition reduces expression of the astrocyte mRNA for at least 8 weeks. In some aspects, a single dose of the oligonucleotide or pharmaceutical composition reduces expression of the astrocyte mRNA for at least 12 weeks. In some aspects, a single dose of the oligonucleotide or pharmaceutical composition reduces expression of the astrocyte mRNA for at least 23 weeks. In some aspects, a single dose of the oligonucleotide or pharmaceutical composition reduces expression of the astrocyte mRNA for at least 26 weeks. In some aspects, a single dose of the oligonucleotide or pharmaceutical composition reduces expression of the astrocyte mRNA for at least 29 weeks.

In any of the foregoing or related aspects of the methods described herein, a single dose of the oligonucleotide or pharmaceutical composition reduces expression of the astrocyte mRNA for up to 4 weeks, up to 8 weeks, up to 12 weeks, up to 23 weeks, up to 26 weeks, or up to 29 weeks. In some aspects, a single dose of the oligonucleotide or pharmaceutical composition reduces expression of the astrocyte mRNA for up to 4 weeks. In some aspects, a single dose of the oligonucleotide or pharmaceutical composition reduces expression of the astrocyte mRNA for up to 8 weeks. In some aspects, a single dose of the oligonucleotide or pharmaceutical composition reduces expression of the astrocyte mRNA for up to 12 weeks. In some aspects, a single dose of the oligonucleotide or pharmaceutical composition reduces expression of the astrocyte mRNA for up to 23 weeks. In some aspects, a single dose of the oligonucleotide or pharmaceutical composition reduces expression of the astrocyte mRNA for up to 26 weeks. In some aspects, a single dose of the oligonucleotide or pharmaceutical composition reduces expression of the astrocyte mRNA for up to 29 weeks.

In any of the foregoing or related aspects of the methods described herein, a single dose of the oligonucleotide or pharmaceutical composition reduces expression of the astrocyte mRNA for up to one year.

In some aspects, the disclosure provides a kit comprising an oligonucleotide described herein, an optional pharmaceutically acceptable carrier, and a package insert comprising instructions for administration to a subject having a disease, disorder or condition associated with expression of an astrocyte mRNA. In some aspects, the package insert comprises instructions for intrathecal administration.

In other aspects, the disclosure provides use of an oligonucleotide or pharmaceutical composition described herein, in the manufacture of a medicament for the treatment of a disease, disorder or condition associated with expression of an astrocyte mRNA.

In further aspects, the disclosure provides an oligonucleotide or pharmaceutical composition described herein for use, or adaptable for use, in the treatment of a disease, disorder or condition associated with expression of an astrocyte mRNA.

The present disclosure is based, at least in part, on the discovery of lipid-conjugated RNAi oligonucleotides that effectively reduce target gene expression in oligodendrocytes of the CNS. Exemplary lipid-conjugated RNAi oligonucleotides provided herein have demonstrated reduction of target gene expression of oligodendrocyte-specific mRNA in the CNS following a single administration. Further, exemplary lipid-conjugated RNAi oligonucleotides provided herein have demonstrated pharmacological activity in multiple regions throughout the CNS, including difficult to reach areas such as the hippocampus and frontal cortex. Without being bound by theory, the hydrophobic moiety (e.g., lipid) facilitates delivery and distribution of the lipid-conjugated RNAi oligonucleotides into the CNS, thereby increasing efficacy and durability of gene knockdown in oligodendrocytes. Accordingly, the disclosure provides methods of treating a disease or disorder by modulating expression of an oligodendrocyte gene in the CNS using the lipid-conjugated RNAi oligonucleotides, and pharmaceutically acceptable compositions thereof, described herein. The disclosure further provides methods of using the lipid-conjugated RNAi oligonucleotides in the manufacture of a medicament for treating a disease or disorder by modulating expression of an oligodendrocyte gene in the CNS.

Accordingly, in some aspects, the disclosure provides a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to an oligodendrocyte mRNA target sequence, and wherein the sense strand comprises at least one lipid moiety conjugated to a nucleotide of the sense strand.

In some aspects, the lipid moiety is selected from

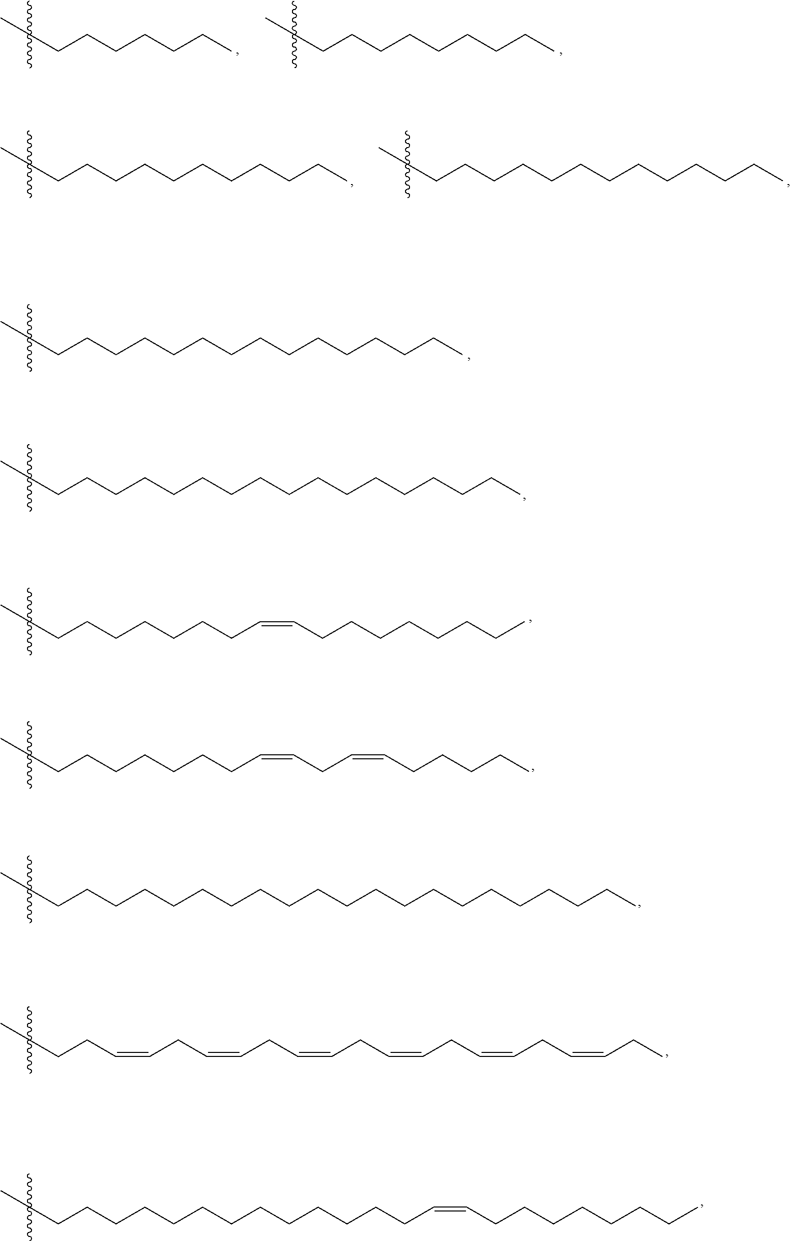

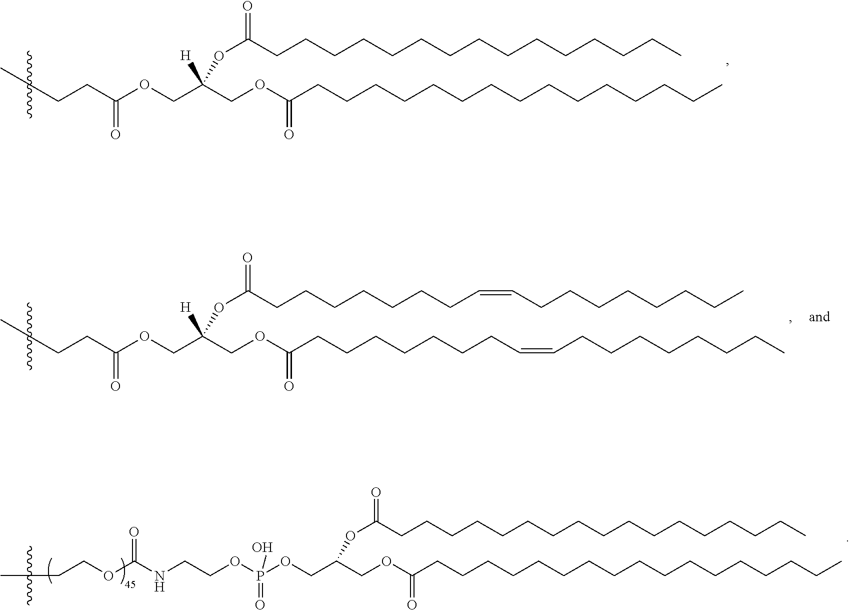

In some aspects, the lipid moiety is a hydrocarbon chain. In some aspects, the hydrocarbon chain is a C8-C30 hydrocarbon chain. In some aspects, the hydrocarbon chain is a C16 hydrocarbon chain. In some aspects, the C16 hydrocarbon chain is represented by

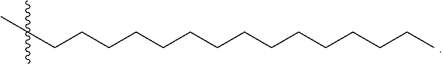 [SMILES: *CCCCCCCCCCCCCCC]

In any of the foregoing or related aspects, the lipid moiety is conjugated to the 2′ carbon of the ribose ring of the nucleotide.

In any of the foregoing or related aspects, the oligonucleotide is blunt ended. In some aspects, the oligonucleotide is blunt ended at the 3′ terminus of the oligonucleotide. In some aspects, the oligonucleotide comprises a blunt end. In some aspects, the blunt end comprises the 3′ terminus of the sense strand. In some aspects, the sense strand is 20-22 nucleotides. In some aspects, the at least one lipid moiety is conjugated to a nucleotide at position 1, position 4, position 8, position 12, position 13, position 18, or position 20 of the sense strand, wherein positions are numbered 5′ to 3′. In some aspects, the oligodendrocyte mRNA target is expressed in the spinal cord, wherein the at least one lipid moiety is conjugated to a nucleotide at position 2, position 3, position 6, position 13, position 14, position 15, position 19 or position 20 of the sense strand, and wherein positions are numbered 5′ to 3′. In some aspects, the oligodendrocyte mRNA target is expressed in the spinal cord, wherein the at least one lipid moiety is conjugated to a nucleotide at position 1, position 2, position 3, position 5, position 6, position 7, position 9, position 13, position 14, position 15, position 17, position 19, or position 20 of the sense strand, and wherein positions are numbered 5′ to 3′. In other aspects, the oligodendrocyte mRNA target is expressed in the medulla, wherein the at least one lipid moiety is conjugated to a nucleotide at position 2, position 14 or position 15 of the sense strand, and wherein positions are numbered 5′ to 3′. In other aspects, the oligodendrocyte mRNA target is expressed in the medulla, wherein the at least one lipid moiety is conjugated to a nucleotide at position 2, position 7, position 9, position 14, position 15, or position 19 of the sense strand, and wherein positions are numbered 5′ to 3′. In other aspects, the oligodendrocyte mRNA target is expressed in the brain stem, wherein the at least one lipid moiety is conjugated to a nucleotide at position 2, position 7, position 9, position 14, position 15, or position 19 of the sense strand, and wherein positions are numbered 5′ to 3′. In further aspects, the oligodendrocyte mRNA target is expressed in the hippocampus, wherein the at least one lipid moiety is conjugated to a nucleotide at position 3 of the sense strand, and wherein positions are numbered 5′ to 3′. In some aspects, the oligodendrocyte mRNA target is expressed in the frontal cortex, wherein the at least one lipid moiety is conjugated to a nucleotide at position 14 of the sense strand, and wherein positions are numbered 5′ to 3′. In some aspects, the oligodendrocyte mRNA target is expressed in the hypothalamus, wherein the at least one lipid moiety is conjugated to a nucleotide at position 7 of the sense strand, and wherein positions are numbered 5′ to 3′.

In any of the foregoing or related aspects, the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2. In some aspects, the sense strand is 36-38 nucleotides. In some aspects, the at least one lipid moiety is conjugated to a nucleotide of the stem-loop or a nucleotide proximal to the stem-loop. In some aspects, the nucleotide proximal to the stem-loop is located 1-3 nucleotides from the 5′ end of the stem-loop. In some aspects, the oligodendrocyte mRNA target is expressed in the spinal cord, wherein the at least one lipid moiety is conjugated to a nucleotide at position 2, position 3, position 6, position 13, position 14, position 15, position 19, position 20, position 23, position 28, position 29 or position 30 of the sense strand, and wherein positions are numbered 5′ to 3′. In other aspects, the oligodendrocyte mRNA target is expressed in the medulla, wherein the at least one lipid moiety is conjugated to a nucleotide at position 19, position 20, position 23 or position 28 of the sense strand, and wherein positions are numbered 5′ to 3′. In further aspects, the oligodendrocyte mRNA target is expressed in the hippocampus, wherein the at least one lipid moiety is conjugated to a nucleotide at position 2 of the sense strand, and wherein positions are numbered 5′ to 3′. In some aspects, the oligodendrocyte mRNA target is expressed in the frontal cortex, wherein the at least one lipid moiety is conjugated to a nucleotide at position 14, position 15, position 19, position 20, position 23, position 28, position 29 or position 30 of the sense strand, and wherein positions are numbered 5′ to 3′.

In any of the foregoing or related aspects, the antisense strand is 22-24 nucleotides. In some aspects, the duplex region is 20-22 base pairs.

In any of the foregoing or related aspects, the antisense strand comprises a 1-4 nucleotide overhang at the 3′ terminus. In some aspects, the overhang comprises purine nucleotides. In some aspects, the overhang sequence is 2 nucleotides in length. In some aspects, the overhang is selected from AA, GG, AG, and GA. In some aspects, the overhang is GG or AA. In some aspects, the overhang is GG.

In any of the foregoing or related aspects, the region of complementarity is complementary to at least 15 consecutive nucleotides of the oligodendrocyte mRNA target sequence. In some aspects, the region of complementarity is complementary to 19 consecutive nucleotides of the oligodendrocyte mRNA target sequence. In some aspects, the region of complementarity is fully complementary to the oligodendrocyte mRNA target sequence. In some aspects, the region of complementarity is partially complementary to the oligodendrocyte mRNA target sequence. In some aspects, the region of complementarity comprises no more than four mismatches to the oligodendrocyte mRNA target sequence. In some aspects, the region of complementarity comprises up to four mismatches to the oligodendrocyte mRNA target sequence.

In any of the foregoing or related aspects, the oligonucleotide comprises at least one modified nucleotide. In some aspects, the modified nucleotide comprises a 2′-modification. In some aspects, each of the nucleotides of the sense strand and the antisense strand comprise a 2′-modification except the nucleotide of the sense strand conjugated to the at least one lipid moiety. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, about 10-20%, 10%, 11%, 12%, 13%, 14% 15%, 16%, 17%, 18%, 19% or 20% of the nucleotides of the sense strand comprise a 2′-fluoro modification. In some aspects, about 25-35%, 25%, 26%, 27%, 28%, 29%, 30%, 31%, 32%, 33%, 34% or 35% of the nucleotides of the antisense strand comprise a 2′-fluoro modification. In some aspects, about 25-35%, 25%, 26%, 27%, 28%, 29%, 30%, 31%, 32%, 33%, 34% or 35% of the nucleotides of the oligonucleotide comprise a 2′-fluoro modification. In some aspects, the sense strand comprises 20 nucleotides with positions 1-20 from 5′ to 3′, wherein each of positions 8-11 comprise a 2′-fluoro modification. In some aspects, the sense strand comprises 20 nucleotides with positions 1-20 from 5′ to 3′, wherein each of positions 9-11 comprise a 2′-fluoro modification. In some aspects, the sense strand comprises 36 nucleotides with positions 1-36 from 5′ to 3′, wherein each of positions 8-11 comprise a 2′-fluoro modification. In some aspects, the sense strand comprises 36 nucleotides with positions 1-36 from 5′ to 3′, wherein each of positions 9-11 comprise a 2′-fluoro modification. In some aspects, the antisense strand comprises 22 nucleotides with positions 1-22 from 5′ to 3′, and wherein each of positions 2, 3, 4, 5, 7, 10 and 14 comprise a 2′-fluoro modification. In some aspects, the remaining nucleotides comprise a 2′-O-methyl modification except the nucleotide of the sense strand conjugated to the at least one lipid moiety.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the sense strand comprises a phosphorothioate linkage between position 1 and 2, wherein positions are numbered 1-2 from 5′ to 3′. In some aspects, the sense strand is 20 nucleotides in length, and wherein the sense strand comprises a phosphorothioate linkage between positions 18 and 19, and between positions 19 and 20, wherein positions are numbered 1-22 from 5′ to 3′.

In any of the foregoing or related aspects, the antisense strand comprises a phosphorylated nucleotide at the 5′ terminus, wherein the phosphorylated nucleotide is selected from uridine and adenosine. In some aspects, the phosphorylated nucleotide is uridine. In some aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate.

In any of the foregoing or related aspects, the region of complementary is fully complementary to the oligodendrocyte mRNA target sequence at nucleotide positions 2-8 of the antisense strand, wherein nucleotide positions are numbered 5′ to 3′. In some aspects, the region of complementary is fully complementary to the oligodendrocyte mRNA target sequence at nucleotide positions 2-11 of the antisense strand, wherein nucleotide positions are numbered 5′ to 3′.

In any of the foregoing or related aspects, the oligonucleotide is a Dicer substrate. In some aspects, the oligonucleotide is a Dicer substrate that, upon endogenous Dicer processing, yields double-stranded nucleic acids of 19-21 nucleotides in length capable of reducing a oligodendrocyte mRNA expression in a mammalian cell.

In any of the foregoing or related aspects, the oligodendrocyte mRNA target sequence is located in a region of the central nervous system (CNS). In some aspects, the region of the CNS is selected from the frontal cortex, spinal cord, lumbar spinal cord, cervical spinal cord, thoracic spinal cord, medulla, cerebellum, hypothalamus, hippocampus, and a combination thereof.

In any of the foregoing or related aspects, the oligonucleotide reduces expression of a target mRNA in an oligodendrocyte or population of oligodendrocytes in vitro and/or in vivo. In some aspects, the disclosure provides a pharmaceutical composition comprising an oligonucleotide described herein, and a pharmaceutically acceptable carrier, delivery agent or excipient.

In other aspects, the disclosure provides a method for treating a subject having a disease, disorder or condition associated with expression of an oligodendrocyte mRNA, the method comprising administering to the subject a therapeutically effective amount of an oligonucleotide or pharmaceutical composition described herein, thereby treating the subject.

In further aspects, the disclosure provides a method of delivering an oligonucleotide to an oligodendrocyte or a population of oligodendrocytes in a subject, the method comprising administering a pharmaceutical composition described herein to the subject. In some aspects, the oligodendrocyte or a population of oligodendrocytes is located in a region of the CNS. In some aspects, the region of the CNS is selected from the frontal cortex, spinal cord, lumbar spinal cord, cervical spinal cord, thoracic spinal cord, medulla, cerebellum, hypothalamus, hippocampus, and a combination thereof.

In yet further aspects, the disclosure provides a method for reducing expression of an oligodendrocyte mRNA in a cell, a population of cells or a subject, the method comprising the step of:
i. contacting the cell or the population of cells with an oligonucleotide or pharmaceutical composition described herein, optionally wherein the cell or population of cells is an oligodendrocyte or a population of oligodendrocytes; or
ii. administering to the subject an oligonucleotide or pharmaceutical composition described herein. In some aspects, reducing expression of the oligodendrocyte mRNA comprises reducing an amount or level of mRNA, an amount or level of protein, or both. In some aspects, the subject has a disease, disorder or condition associated with expression of the oligodendrocyte mRNA. In some aspects, the cell or population of cells is located in a region of the CNS. In some aspects, the region of the CNS is selected from the frontal cortex, spinal cord, lumbar spinal cord, cervical spinal cord, thoracic spinal cord, medulla, cerebellum, hypothalamus, hippocampus, and a combination thereof.

In any of the foregoing or related aspects, the methods comprise administering via intrathecal administration.

In some aspects, the disclosure provides a method of reducing expression of a target mRNA expressed in an oligodendrocyte in a tissue of the CNS of a subject, comprising administering to the subject a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a target sequence in the target mRNA, and wherein the sense strand comprises at least one lipid moiety conjugated to a nucleotide of the sense strand. In some aspects, the lipid moiety is a C16 hydrocarbon.

In any of the foregoing or related aspects of the methods described herein, the oligonucleotide is blunt ended at the 3′ terminus of the oligonucleotide. In some aspects, the oligonucleotide comprises a blunt end. In some aspects, the blunt end comprises the 3′ terminus of the sense strand. In some aspects, the sense strand is 22-24 nucleotides. In some aspects, the tissue is the spinal cord, wherein the at least one lipid moiety is conjugated to a nucleotide at position 2, position 3, position 6, position 13, position 14, position 15, position 19 or position 20 of the sense strand, and wherein positions are numbered 5′ to 3′. In other aspects, the tissue is the spinal cord, wherein the at least one lipid moiety is conjugated to a nucleotide at position 1, position 2, position 3, position 5, position 6, position 7, position 9, position 13, position 14, position 15, position 17, position 19, or position 20 of the sense strand, and wherein positions are numbered 5′ to 3′. In other aspects, the tissue is the medulla, wherein the at least one lipid moiety is conjugated to a nucleotide at position 2, position 14 or position 15 of the sense strand, and wherein positions are numbered 5′ to 3′. In other aspects, the tissue is the medulla, wherein the at least one lipid moiety is conjugated to a nucleotide at position 2, position 7, position 14, position 15, or position 19 of the sense strand, and wherein positions are numbered 5′ to 3′. In other aspects, the tissue is the brain stem, wherein the at least one lipid moiety is conjugated to a nucleotide at position 2, position 7, position 14, position 15, or position 19 of the sense strand, and wherein positions are numbered 5′ to 3′. In further aspects, the tissue is the hippocampus, wherein the at least one lipid moiety is conjugated to a nucleotide at position 3 of the sense strand, and wherein positions are numbered 5′ to 3′. In some aspects, the tissue is the frontal cortex, wherein the at least one lipid moiety is conjugated to a nucleotide at position 14 of the sense strand, and wherein positions are numbered 5′ to 3′. In some aspects, the tissue is the hypothalamus, wherein the at least one lipid moiety is conjugated to a nucleotide at position 7 of the sense strand, and wherein positions are numbered 5′ to 3′.

In any of the foregoing or related aspects of the methods described herein, the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2. In some aspects, the sense strand is 36-38 nucleotides. In some aspects, the at least one lipid moiety is conjugated to a nucleotide of the stem-loop or a nucleotide proximal to the stem-loop. In some aspects, the nucleotide proximal to the stem-loop is located 1-3 nucleotides from the 5′ end of the stem-loop. In some aspects, the tissue is the spinal cord, wherein the at least one lipid moiety is conjugated to a nucleotide at position 2, position 3, position 6, position 13, position 14, position 15, position 19, position 20, position 23, position 28, position 29 or position 30 of the sense strand, and wherein positions are numbered 5′ to 3′. In other aspects, the tissue is the medulla, wherein the at least one lipid moiety is conjugated to a nucleotide at position 19, position 20, position 23 or position 28 of the sense strand, and wherein positions are numbered 5′ to 3′. In further aspects, the tissue is the hippocampus, wherein the at least one lipid moiety is conjugated to a nucleotide at position 2 of the sense strand, and wherein positions are numbered 5′ to 3′. In some aspects, the tissue is the frontal cortex, wherein the at least one lipid moiety is conjugated to a nucleotide at position 14, position 15, position 19, position 20, position 23, position 28, position 29 or position 30 of the sense strand, and wherein positions are numbered 5′ to 3′.

In other aspects, the disclosure provides a kit comprising an oligonucleotide described herein, an optional pharmaceutically acceptable carrier, and a package insert comprising instructions for administration to a subject having a disease, disorder or condition associated with expression of an oligodendrocyte mRNA. In some aspects, the package insert comprises instructions for intrathecal administration.

In further aspects, the disclosure provides use of an oligonucleotide or pharmaceutical composition described herein, in the manufacture of a medicament for the treatment of a disease, disorder or condition associated with expression of an oligodendrocyte mRNA.

In some aspects, the disclosure provides an oligonucleotide or pharmaceutical composition described herein for use, or adaptable for use, in the treatment of a disease, disorder or condition associated with expression of an oligodendrocyte mRNA.

### BRIEF DESCRIPTION OF FIGURES

FIGS. 1A-1C provide schematics of RNAi oligonucleotide-lipid conjugates targeting TUBB3 mRNA having the structures of Compounds 1-14.

FIGS. 2A-2F provide graphs measuring percent (%) of murine TUBB3 mRNA remaining in frontal cortex (FIG. 2A), hippocampus (FIG. 2B), cerebellum (FIG. 2C), brain stem (FIG. 2D), lumbar dorsal root ganglion (DRG) (FIG. 2E), and lumbar spinal cord (FIG. 2F) of control mice (group A) or mice administered Compounds 1-14 (respectively groups B-O) via lumbar intrathecal injection.

FIG. 3 provides a schematic of RNAi oligonucleotide-lipid conjugates targeting TUBB3 mRNA having the structures of Compounds 15-18.

FIGS. 4A-4F provide graphs measuring percent (%) of murine TUBB3 mRNA remaining in frontal cortex (FIG. 4A), hippocampus (FIG. 4B), medulla (FIG. 4C) cerebellum (FIG. 4D), lumbar dorsal root ganglion (DRG) (FIG. 4E), and lumbar spinal cord (FIG. 4F) of control mice (group A) or mice administered Compounds 7-9 (respectively groups B-D) or Compounds 15-18 (respectively groups E-H) via lumbar intrathecal injection.

FIG. 5 provides a schematic of RNAi oligonucleotide-lipid conjugates targeting TUBB3 mRNA having the structures of Compounds 18-28.

FIGS. 6A-6B provide graphs measuring percent (%) of murine TUBB3 mRNA remaining in cerebellum (FIG. 6A) and lumbar dorsal root ganglion (DRG) (FIG. 6B) of control mice (group A) or mice administered Compounds 18-28 (respectively groups B-L).

FIG. 7 provides a schematic of RNAi oligonucleotide-lipid conjugates targeting TUBB3 mRNA having the structures of Compounds 18 and 29-38.

FIGS. 8A-8B provide graphs measuring percent (%) of murine TUBB3 mRNA remaining in cerebellum (FIG. 8A) and lumbar dorsal root ganglion (DRG) (FIG. 8B) of control mice (group A) or mice administered Compounds 18 or 29-38 (respectively groups B-L).

FIG. 9 provides a schematic of RNAi oligonucleotide-lipid conjugates targeting reticulon 4 (RTN4) mRNA having the structures of Compounds 39-49.

FIGS. 10A-10B provide graphs measuring percent (%) of rat RTN4 mRNA remaining in retina (FIG. 10A) and optic nerve (FIG. 8B) tissues of control rats (group A) or rats administered Compounds 39-49 (respectively groups B-L) via intravitreal injection.

FIG. 10C provides a schematic of RNAi oligonucleotide-GalNAc conjugates targeting Aldh2 mRNA having the structures of Compounds 57 and 98-108.

FIG. 10D provides a graph measuring percent (%) of murine Aldh2 mRNA remaining in liver tissue of control mice administered PBS (group A) or mice administered Compounds 57 or 98-108 (respectively groups B-N).

FIG. 10E provides a schematic of RNAi oligonucleotide-lipid conjugates targeting PECAM-1 mRNA having the structures of Compounds 120 and 121 and CD68 mRNA having the structure of Compound 122.

FIGS. 10F-10G provide graphs measuring percent (%) of murine PECAM mRNA (FIG. 10F) and CD68 mRNA (FIG. 10G) remaining in liver tissue of control mice administered PBS (group A) or mice administered Compounds 120-122 (respectively groups B-D) via subcutaneous injection.

FIGS. 10H-10I provide graphs measuring percent (%) of murine CD68 mRNA (FIG. 10H) and CD68 protein (FIG. 10I) remaining in liver tissue of control mice administered PBS or mice administered Compound 122 at the indicated dose via subcutaneous injection, as measured by qPCR and immunohistochemistry (IHC) respectively.

FIG. 10J provides a representative image of liver tissue obtained from a PBS control mice as described in FIGS. 10H-10I that were imaged following CD68 immunostaining. Arrows indicate representative cells that morphologically present as hepatocytes or macrophages. Dark staining indicates CD68 protein expression.

FIG. 10K provides representative images of liver tissue obtained from mice described in FIGS. 10H-10I that were imaged following CD68 immunostaining. Dark staining indicates CD68 protein expression.

FIGS. 11A-11B provide schematics of RNAi oligonucleotide-lipid conjugates targeting CD68 mRNA having the structures of Compounds 58-97.

FIG. 12 provides a graph measuring percent (%) of murine CD68 mRNA remaining in liver tissue of control mice administered PBS or mice administered Compounds 58-97 (respectively groups B-AO) via subcutaneous injection.

FIG. 13 provides a schematic of RNAi oligonucleotide-lipid conjugates targeting Aldh2 mRNA having the structures of Compounds 109-117.

FIGS. 14A-14D provide graphs measuring percent of murine Aldh2 mRNA remaining in liver (FIG. 14A), adipose (FIG. 14B), skeletal muscle (FIG. 14C), and adrenal (FIG. 14D) tissues in control mice administered PBS (group A) or mice administered Compounds 109-117 (respectively groups B-J) via subcutaneous injection.

FIG. 15 provides a schematic of RNAi oligonucleotide-lipid conjugates targeting STAT3 (Compounds 123 and 127), SLC25A1 (Compounds 124 and 128), HMGB1 (Compounds 125 and 129), and ALDH2 (Compounds 126 and 130).

FIGS. 16A-16D provide graphs measuring percent (%) of murine STAT3 mRNA remaining in liver tissue of mice administered PBS (group A) or compounds 123 or 127 (groups B1 and C1 respectively) (FIG. 16A); percent (%) of murine SLC25A1 mRNA remaining in liver tissue of mice administered PBS (group A) or compounds 124 or 128 (groups B2 and C2 respectively) (FIG. 16B); percent (%) of murine HMGB1 mRNA remaining in liver tissue of mice administered PBS (group A) or compounds 125 or 129 (groups B3 and C3 respectively) (FIG. 16C); and percent (%) of murine ALDH2 mRNA remaining in liver tissue of mice administered PBS (group A) or compounds 126 or 130 (groups B4 and C4 respectively) (FIG. 16D). Mice were administered RNAi oligonucleotide-lipid conjugates via subcutaneous injection.

FIG. 17 provides a schematic of RNAi oligonucleotide-lipid conjugates targeting TUBB3 where the oligonucleotide comprises a P-4, P-6, or P-8 truncated sense strand (i.e. a 6, 8, and 10 nucleotide overhang of the antisense strand) (Compounds 131-133) or a p-4, p-6, or p-8 truncated sense strand comprising phosphorothioate linkages (Compounds 134-136).

FIGS. 18A-18F provide graphs measuring percent (%) of murine TUBB3 mRNA remaining in frontal cortex (FIG. 18A), hippocampus (FIG. 18B), cerebellum (FIG. 18C), lumbar dorsal root ganglion (DRG) (FIG. 18D), medulla (FIG. 18E), and lumbar spinal cord (SC) (FIG. 18F) of control mice (aCSF) or mice administered Compounds 131-136 (respectively Parent C16, P-4, P-6, P-8, P-4 PS, P-6 PS, and P-8 PS) via lumbar intrathecal injection.

FIG. 19 provides a schematic of blunt-end RNAi oligonucleotide-lipid conjugates targeting TUBB3 where the oligonucleotide comprises a sense strand with different amounts of locked nucleic acids (LNA) (Compounds 137-140) or a P-6 truncated sense strand (i.e., an 8 nucleotide overhang of the antisense strand) comprising different amounts of LNAs (Compounds 141-145).

FIGS. 20A-20F provide graphs measuring percent (%) of murine TUBB3 mRNA remaining in frontal cortex (FIG. 20A), hippocampus (FIG. 20B), cerebellum (FIG. 20C), medulla (FIG. 20D), lumbar dorsal root ganglion (DRG) (FIG. 20E), and lumbar spinal cord (SC) (FIG. 20F) of control mice (aCSF) or mice administered Compounds 1 and 137-145 (respectively B-K) via lumbar intrathecal injection.

FIG. 21 provides a schematic of blunt-end RNAi oligonucleotide-lipid conjugates targeting TUBB3 where the oligonucleotide comprises a sense strand comprising different amounts of locked nucleic acids (LNA) (Compounds 138, 139, 140, 147, and 148).

FIGS. 22A-22F provide graphs measuring percent (%) of murine TUBB3 mRNA remaining in frontal cortex (FIG. 22A), hippocampus (FIG. 22B), medulla (FIG. 22C), lumbar dorsal root ganglion (DRG) (FIG. 22D), cerebellum (FIG. 22E), and lumbar spinal cord (SC) (FIG. 22F) of control mice (aCSF) or mice administered Compounds 1, 137, 146, 138, 139, 140, 147, and 148 (respectively B-I) via lumbar intrathecal injection.

FIG. 23 provides a schematic of blunt-end RNAi oligonucleotide-lipid conjugates targeting TUBB3 where the oligonucleotide comprises a P-4 truncated sense strand comprising different amounts of locked nucleic acids (LNA) (Compounds 149-154).

FIGS. 24A-24F provide graphs measuring percent (%) of murine TUBB3 mRNA remaining in frontal cortex (FIG. 24A), hippocampus (FIG. 24B), medulla (FIG. 24C), lumbar dorsal root ganglion (DRG) (FIG. 24D), cerebellum (FIG. 24E), and lumbar spinal cord (SC) (FIG. 24F) of control mice (aCSF) or mice administered Compounds 1, 137 and 149-154 (respectively B-I) via lumbar intrathecal injection.

FIG. 25 provides a schematic of blunt-end RNAi oligonucleotide-lipid conjugates targeting TUBB3 where the oligonucleotide comprises a P-8 truncated sense strand comprising different amounts of locked nucleic acids (LNA) (Compounds 155-160).

FIGS. 26A-26F provide graphs measuring percent (%) of murine TUBB3 mRNA remaining in frontal cortex (FIG. 26A), hippocampus (FIG. 26B), medulla (FIG. 26C), lumbar dorsal root ganglion (DRG) (FIG. 26D), cerebellum (FIG. 26E), and lumbar spinal cord (SC) (FIG. 26F) of control mice (aCSF) or mice administered Compounds 1, 137 and 155-160 (respectively B-I) via lumbar intrathecal injection.

FIG. 27 provides a schematic of blunt-end RNAi oligonucleotide-lipid conjugates targeting UGT8 where the oligonucleotide comprises a sense strand comprising different amounts of locked nucleic acids (LNA) (Compounds 162-165) or a P-6 truncated sense strand comprising different amounts of LNAs (Compounds 166-171).

FIGS. 28A-28F provide graphs measuring percent (%) of murine UGT8 mRNA remaining in frontal cortex (FIG. 28A), hippocampus (FIG. 28B), hypothalamus (FIG. 28C), cerebellum (FIG. 28D), medulla (FIG. 28E), and lumbar spinal cord (FIG. 26F) of control mice (aCSF) or mice administered Compounds 161-171 (respectively B-L) via lumbar intrathecal injection.

FIG. 29 provides a schematic of blunt-end RNAi oligonucleotide-lipid conjugates targeting GFAP where the oligonucleotide comprises a sense strand comprising different amounts of locked nucleic acids (LNA) (Compounds 173-176) or a P-6 truncated sense strand comprising different amounts of LNAs (Compounds 177-182).

FIGS. 30A-30F provide graphs measuring percent (%) of murine GFAP mRNA remaining in frontal cortex (FIG. 30A), hippocampus (FIG. 30B), cerebellum (FIG. 30C), medulla (FIG. 30D), hypothalamus (FIG. 30E), and lumbar spinal cord (FIG. 30F) of control mice (aCSF) or mice administered Compounds 172-182 (respectively B-L) via lumbar intrathecal injection.

FIGS. 31A-31D provide schematics of blunt-end RNAi oligonucleotide-lipid conjugates targeting TUBB3 where the oligonucleotide comprises different sense strand truncations including: no truncation (FIG. 31A; Compounds 137 and 183-190); a P-4 truncated sense strand (FIG. 31B; Compounds 149, and 191-194); a P-6 truncated sense strand (FIG. 31C; Compounds 141, 195, and 196); and, a P-8 truncated sense strand (FIG. 31D; Compound 197). Each compound comprises a C16 lipid conjugated to a different position of the sense strand as indicated in the schematics.

FIGS. 32A-32F provide graphs measuring percent (%) of murine TUBB3 mRNA remaining in frontal cortex (FIG. 32A), hippocampus (FIG. 32B), medulla (FIG. 32C), lumbar dorsal root ganglion (FIG. 32D), cerebellum (FIG. 32E), and lumbar spinal cord (FIG. 32F) of control mice (aCSF) or mice administered Compounds 137, 183-190, 149, 191-194, 141, and 195-197 via lumbar intrathecal injection.

FIGS. 33A-33C provide schematics of RNAi oligonucleotide-lipid conjugates targeting GFAP mRNA having the structures of Compounds 173, 176, 180, 200-205, and 207-215.

FIGS. 34A-34D provide graphs measuring percent (%) of murine GFAP mRNA remaining in frontal cortex (FIG. 34A), hippocampus (FIG. 34B), medulla (FIG. 34C), and lumbar spinal cord (FIG. 34D) of control mice (aCSF) or mice administered compounds 173, 176, 180, 200-205, and 207-215 (as identified in Table 20) via lumbar intrathecal injection.

FIGS. 35A-35E provide schematics of RNAi oligonucleotide-lipid conjugates targeting TUBB3 mRNA having the structures of Compounds 137, 140, 144, 217-222, 224-232, and 277-285.

FIGS. 36A-36D provide graphs measuring percent (%) of murine TUBB3 mRNA remaining in frontal cortex (FIG. 34A), hippocampus (FIG. 34B), medulla (FIG. 34C), and lumbar spinal cord (FIG. 34D) of control mice (aCSF) or mice administered compounds 137, and 140, 144, 217-222, 224-232 (as identified in Table 21) via lumbar intrathecal injection.

FIG. 36E is a graph measuring (%) of murine TUBB3 mRNA remaining in frontal cortex hippocampus, hypothalamus, cerebellum, brain stem, and lumbar spinal cord of control mice (aCSF) or mice administered compounds 140, 277-278, 231, and 279-285 (respectively B-L) via lumbar intrathecal injection.

FIGS. 37A-37B provide schematics of RNAi oligonucleotide-lipid conjugates targeting GFAP mRNA having the structures of Compounds 173, 176, and 233-245.

FIGS. 38A-38D provide graphs measuring percent (%) of murinc GFAP mRNA remaining in frontal cortex (FIG. 38A), hippocampus (FIG. 38B), medulla (FIG. 38C), and lumbar spinal cord (FIG. 38D) of control mice (aCSF) or mice administered compounds 173, 176, and 233-245 (respectively B-P) via lumbar intrathecal injection.

FIGS. 39A-39B provide schematics of RNAi oligonucleotide-lipid conjugates targeting GFAP mRNA having the structures of Compounds 173, 176, 200 and 246-255.

FIGS. 40A-40D provide graphs measuring percent (%) of murine GFAP mRNA remaining in frontal cortex (FIG. 40A), hippocampus (FIG. 40B), medulla (FIG. 40C), and lumbar spinal cord (FIG. 40D) of control mice (aCSF) or mice administered compounds 173, 176, 200 and 246-255 (respectively B-N) via lumbar intrathecal injection.

FIGS. 41A-41B provide schematics of RNAi oligonucleotide-lipid conjugates targeting TUBB3 mRNA having the structures of Compounds 137, 140, and 257-268.

FIGS. 42A-42D provide graphs measuring percent (%) of murine TUBB3 mRNA remaining in frontal cortex (FIG. 42A), hippocampus (FIG. 42B), medulla (FIG. 42C), and lumbar spinal cord (FIG. 42D) of control mice (aCSF) or mice administered compounds 137, 140, and 257-268 (respectively B-P) via lumbar intrathecal injection.

FIGS. 43A-43B provide schematics of RNAi oligonucleotide-lipid conjugates targeting TUBB3 mRNA having the structures of Compounds 137, 140, 217, and 269-276.

FIGS. 44A-44D provide graphs measuring percent (%) of murine TUBB3 mRNA remaining in frontal cortex (FIG. 44A), hippocampus (FIG. 44B), medulla (FIG. 44C), and lumbar spinal cord (FIG. 44D) of control mice (aCSF) or mice administered compounds 137, 140, 217, and 269-276 (respectively B-F and I-N) via lumbar intrathecal injection.

FIG. 45 provides schematics of RNAi oligonucleotide-lipid conjugates targeting GFAP mRNA having the structures of Compounds 286-292.

FIGS. 46A-46D provide graphs measuring percent (%) of murine ALDH2 mRNA remaining in liver (FIG. 46A), quadricep (quad) muscle (FIG. 46B), heart muscle (FIG. 46C), and gonadal white adipose tissue (gWAT) (FIG. 46D) of control mice (PBS) or mice administered compounds 286-292 (respectively A-G) via subcutaneous injection.

FIG. 47 provides schematics of RNAi oligonucleotide-lipid conjugates targeting UGT8 mRNA having the structures of Compounds 162, 167, 293, and 294.

FIGS. 48A-48D provide graphs measuring percent (%) of murine UGT8 mRNA remaining in frontal cortex (FIG. 48A), hippocampus (FIG. 48B), brain stem (FIG. 48C), and lumbar spinal cord (FIG. 48D) of control mice (aCSF) or mice administered compounds 162, 167, 293, and 294 (respectively blunt, 5′ p-6, 3′ p-3, and 5′ p-6 & 3′ p-3) via lumbar intrathecal injection.

FIGS. 49A-49F provide graphs depicting the concentration response of a GalNAc-conjugated oligonucleotide targeting the astrocyte-specific gene GFAP. The percent (%) of murine Gfap mRNA remaining in cervical spinal cord (FIG. 49A), thoracic spinal cord (FIG. 49B), lumbar spinal cord (FIG. 49C), frontal cortex (FIG. 49D), hippocampus (FIG. 49E), and cerebellum (FIG. 49F) of mice after treatment with GalNAc-conjugated Gfap oligonucleotide (Table 29) was determined. Mice were treated with 10, 32, 100, 320, or 1000 μg the GalNAc-conjugated Gfap oligonucleotide formulated in artificial cerebrospinal fluid (aCSF) via intrathecal injection into the lumbar spine. Seven (7) days following intrathecal injection, the level of Gfap mRNA was normalized to Ribosomal Protein L23 (RPL23) mRNA and overall expression was determined between tissue types relative to control mice treated with aCSF.

FIG. 50 provides a graph depicting the average percent (%) of murine Gfap mRNA remaining in different tissues of the central nervous system (CNS) based on the results in FIGS. 49A-49F and the resulting EC₅₀ (ED₅₀) calculated for each CNS tissue.

FIGS. 51A-51D provide graphs depicting the concentration-response of a GalNAc-conjugated Gfap oligonucleotide. The percent (%) of murine Gfap mRNA remaining in frontal cortex (FIG. 51A), brain stem (FIG. 51B), hippocampus (FIG. 51C), and lumbar spinal cord (FIG. 51D), of mice after treatment with GalNAc-conjugated Gfap oligonucleotide (Table 29) was determined. Mice were treated with 10, 32, 100, or 300 μg of the oligonucleotide formulated in artificial cerebrospinal fluid (aCSF) via intracerebroventricular (i.c.v) injection. Seven (7) days following i.c.v. injection, the level of Gfap mRNA was normalized to Ribosomal Protein L23 (RPL23) mRNA and overall expression was determined between tissue types relative to control mice treated with aCSF.

FIG. 52 provides a graph depicting the average percent (%) of murine Gfap mRNA remaining in different tissues of the CNS based on the results in FIGS. 51A-51D and the resulting EC₅₀ (ED₅₀) calculated for each CNS tissue.

FIGS. 53A-53B provide graphs depicting the percent (%) of rat Gfap mRNA remining in tissues of the central nervous system of rats after treatment with C16 lipid-conjugated (FIG. 53A) or GalNAc-conjugated “GalXC” (FIG. 53B) Gfap tetraloop oligonucleotides. Rats were treated with 1000 μg of the indicated Gfap oligonucleotides in Table 30 formulated in artificial cerebrospinal fluid (aCSF) via intrathecal injection to the lumbar spine. At 8, 12, or 23 weeks post administration, the level of Gfap mRNA was normalized to peptidylprolyl Isomerase B (Ppib) mRNA and overall expression was determined between tissue types relative to control rats treated with aCSF.

FIG. 54 provides a graph depicting the percent (%) of rat Gfap mRNA remining in tissues of the central nervous system of rats after treatment with a C16 lipid-conjugated Gfap tetraloop oligonucleotide. Rats were treated with 1000 μg of the indicated Gfap lipid-conjugated tetraloop oligonucleotide in Table 30 formulated in artificial cerebrospinal fluid (aCSF) via intracisternal magna injection. At 4- or 12-weeks post administration, the level of Gfap mRNA was normalized to peptidylprolyl Isomerase B (Ppib) mRNA and overall expression was determined between tissue types relative to control rats treated with aCSF.

FIGS. 55A-55B provide graphs depicting the percent (%) of rat Gfap mRNA remining in tissues of the central nervous system of rats after treatment with a C16 lipid-conjugated Gfap tetraloop oligonucleotide. Rats were treated with 1000 μg of the indicated Gfap lipid-conjugated tetraloop oligonucleotide in Table 30 formulated in artificial cerebrospinal fluid (aCSF) via intracisternal magna injection. At 26 weeks (FIG. 55A) or 39 weeks (FIG. 55B) post administration, the level of Gfap mRNA was normalized to peptidylprolyl Isomerase B (Ppib) mRNA and overall expression was determined between tissue types relative to control rats treated with aCSF.

FIGS. 56A-56F provide graphs depicting the percent (%) of murine Gfap mRNA remaining in lumbar spinal cord (FIG. 56A), medulla (FIG. 56B), cerebellum (FIG. 56C), hypothalamus (FIG. 56D), hippocampus (FIG. 56E), and frontal cortex (FIG. 56F) of mice after treatment with Gfap tetraloop oligonucleotides having a lipid conjugated at a nucleotide position indicated on the x-axis. Mice were treated with 300 μg of the indicated Gfap lipid-conjugated tetraloop oligonucleotides in Table 31 formulated in artificial cerebrospinal fluid (aSCF) via intrathecal (i.t.) injection. Seven (7) days post dose, the level of Gfap mRNA was normalized to Ribosomal Protein L23 (RPL23) mRNA and overall expression was determined between tissue types relative to control mice treated with aCSF.

FIGS. 57A-57F provide graphs depicting the percent (%) of murine Gfap mRNA remaining in lumbar spinal cord (FIG. 57A), medulla (FIG. 57B), cerebellum (FIG. 57C), hypothalamus (FIG. 57D), hippocampus (FIG. 57E), and frontal cortex (FIG. 57F) of mice after treatment with Gfap blunt-end or tetraloop oligonucleotides having a lipid conjugated at a nucleotide position indicated on the x-axis. Mice were treated with 300 μg of the indicated C16 lipid-conjugated Gfap oligonucleotides in Table 32 formulated in artificial cerebrospinal fluid (aCSF) via intrathecal injection into the lumbar spine. Seven (7) days following intrathecal injection, the level of Gfap mRNA was normalized to Ribosomal Protein L23 (RPL23) mRNA and overall expression was determined between tissue types relative to control mice treated with aCSF.

FIGS. 58A-58F provide graphs depicting the percent (%) of murine Gfap mRNA remaining in lumbar spinal cord (FIG. 58A), medulla (FIG. 58B), cerebellum (FIG. 58C), hypothalamus (FIG. 58D), hippocampus (FIG. 58E), and frontal cortex (FIG. 58F) of mice after treatment with lipid-conjugated Gfap blunt-end or tetraloop oligonucleotides as assessed in FIGS. 56A-56F and 57A-57F. Experiment 1 represents the oligonucleotides assessed in FIGS. 56A-56F. Experiment 2 represents the tetraloop oligonucleotides assessed in FIGS. 57A-57F. Experiment 3 represents the blunt-end oligonucleotides assessed in FIGS. 57A-57F.

FIGS. 59A-59B provide graphs depicting the concentration response of C16-conjugated Gfap blunt-end oligonucleotides. The percent (%) of murine Gfap mRNA remaining in the CNS of mice after treatment with a C16-conjugated Gfap blunt-end oligonucleotide. Mice were treated with 3, 10, 30, 100, or 300 μg of the indicated oligonucleotide formulated in artificial cerebrospinal fluid (aCSF) via intrathecal injection into the lumbar spine. Seven (7) days (FIG. 59A) and 28-days (FIG. 59B) following intrathecal injection, the level of Gfap mRNA was normalized to Ribosomal Protein L23 (RPL23) mRNA and overall expression and ED₅₀ was determined between tissue types relative to control mice treated with aCSF.

FIG. 60 provides a graph depicting the concentration response of GalNAc-conjugated UGT8 oligonucleotides. The percent (%) of human UGT8 mRNA remaining in the liver of mice exogenously expressing human UGT8 (hydrodynamic injection model) after treatment with GalNAc-conjugated UGT8 oligonucleotides (Table 34) at two concentrations (0.3 mg/kg and 1 mg/kg) was measured. Three days following administration, mice were hydrodynamically injected (HDI) with a DNA plasmid encoding human UGT8. The level of human UGT8 mRNA was determined from livers collected 18 hours later. The x-axis indicates the oligonucleotide administered and the concentration (e.g., administration of UGT8-277 at 0.3 mg/kg is shown as “277-0.3”).

FIGS. 61A-61F provide graphs depicting the concentration response of a UGT8 oligonucleotide having a 2′OMe tetraloop in the central nervous system (CNS). The percent (%) of murine UGT8 mRNA remaining in lumbar spinal cord (FIG. 61A), cervical spinal cord (FIG. 61B), brainstem (FIG. 61C), cerebellum (FIG. 61D), hypothalamus (FIG. 61E), and frontal cortex (FIG. 61F) was determined. Mice were treated with 10, 32, 100, 320, or 500 μg of UGT8-277 oligonucleotide formulated in artificial cerebrospinal fluid (aCSF) via intrathecal (i.t.) injection was measured. Seven (7) days following intrathecal injection, the level of UGT8 mRNA was measured and normalized to Ribosomal Protein L23 (RPL23) mRNA and overall expression was determined between tissue types relative to control mice treated with (aCSF). ‘GalXC-UGT8’ refers to UGT8-277 oligonucleotide having SEQ ID NO: 841 (sense strand) and SEQ ID NO: 831 (antisense strand).

FIG. 62 provides a graph depicting the percent (%) murine UGT8 mRNA remaining in different tissues of the central nervous system (CNS) based on the results in FIGS. 61A-61F.

FIGS. 63A-63D provide graphs depicting the percent (%) murine UGT8 mRNA remaining in lumbar spinal cord (FIG. 63A), medulla (FIG. 63B), hippocampus (FIG. 63C), and frontal cortex (FIG. 63D) of mice after treatment with lipid-conjugated UGT8 tetraloop oligonucleotides. Mice were treated with 300 μg of the indicated UGT8 lipid-conjugated tetraloop oligonucleotides in Table 35 formulated in artificial cerebrospinal fluid (aCSF) via intrathecal (i.t.) injection into the lumbar spine. Seven (7) days following intrathecal injection, the level of UGT8 mRNA was normalized to Ribosomal Protein L23 (RPL23) mRNA and overall expression was determined between tissue types relative to control mice treated with (aCSF).

FIG. 64 provides schematics of UGT8 lipid-conjugated blunt-end oligonucleotides having the structures of the compounds in Table 36. The nucleotide position on the sense strand having a conjugated lipid is indicated as “P #”, e.g., a lipid conjugated at position 2 of the sense strand from 5′ to 3′ is indicated as “P2”.

FIGS. 65A-65D provide graphs depicting the percent (%) murine UGT8 mRNA remaining in lumbar spinal cord (FIG. 65A), medulla (FIG. 65B), hippocampus (FIG. 65C), and frontal cortex (FIG. 65D) of mice after treatment with the lipid-conjugated UGT8 blunt-end oligonucleotides of FIG. 64. Mice were treated with 300 μg of the indicated UGT8 lipid-conjugated blunt-end oligonucleotides in Table 36 formulated in artificial cerebrospinal fluid (aCSF) via intrathecal injection into the lumbar spine. Seven (7) days following intrathecal injection, the level of UGT8 mRNA was normalized to Ribosomal Protein L23 (RPL23) mRNA and overall expression was determined between tissue types relative to control mice treated with (aCSF).

FIGS. 66A-66B provide schematics of UGT8 lipid-conjugated blunt-end oligonucleotides having the structures of the compounds in Table 37. The nucleotide position on the sense strand having a conjugated lipid is indicated as “P #”, e.g., a lipid conjugated at position 1 of the sense strand from 5′ to 3′ is indicated as “P1”.

FIGS. 67A-67E provide graphs depicting the percent (%) murine UGT8 mRNA remaining in frontal cortex (FIG. 67A), hippocampus (FIG. 67B), hypothalamus (FIG. 67C), brainstem (FIG. 67D), and lumbar spinal cord (FIG. 67E) of mice after treatment with lipid-conjugated UGT8 blunt-end oligonucleotides. Mice were treated with 300 μg of the indicated UGT8 lipid-conjugated blunt-end oligonucleotides in Table 37 formulated in artificial cerebrospinal fluid (aCSF) via intrathecal injection into the lumbar spine. Twenty-eight (28) days following intrathecal injection, the level of UGT8 mRNA was normalized to Ribosomal Protein L23 (RPL23) mRNA and overall expression was determined between tissue types relative to control mice treated with (aCSF).

### DETAILED DESCRIPTION

In some aspects, the disclosure provides oligonucleotide-lipid conjugates (e.g., RNAi oligonucleotide-lipid conjugates) that reduce expression of a target gene. In other aspects, the disclosure provides methods of treating a disease or disorder associated with expression of a target gene. In other aspects, the disclosure provides methods of treating a disease or disorder (e.g., a neurological disease and/or by inappropriate gene expression) associated with expression of a target gene using the lipid-conjugated RNAi oligonucleotides, or pharmaceutically acceptable compositions thereof, described herein. In other aspects, the disclosure provides methods of using the lipid-conjugated RNAi oligonucleotides described herein in the manufacture of a medicament for treating a disease or disorder associated with expression of a target gene.

In nucleic acid chemistry, many different artificial nucleic acids have been developed to alter the behavior of siRNAs under physiological conditions. In particular, phosphorothioate (PS), 2′-methoxy (2′-OMe), and 2′-fluoro nucleic acid have often been used to modify the siRNA its behavior, toxicity and thermostability. Recently a novel class of conformationally restricted artificial nucleic acids has been developed these are bridged nucleic acids (BNAs). The basic structure of the BNA is the bridged structure between 2′-O and 4′-C which fixes the furanose ring to the N-type and 2′,4′-BNA gives its nucleotide analog high nuclease resistance and affinity to complementary RNA. The concept of introducing a bulky group at the 2′-position of the furanose ring to achieve high nuclease resistance has little impact on sugar fluctuation, whereas in the BNAs, the bicyclic structure provides steric hindrance at the phosphodiester backbone and the reduction of entropic loss by the sugar fluctuation, and thus both high nuclease resistance and stabilization of the RNA duplex are realized. Many studies related to usage of 2′,4′-BNA/locked nucleic acid (LNA) modifications in siRNAs have been conducted in vitro. Similarly, locked nucleic acids (LNA's) are a class of nucleic acid analogues that display increased hybridization affinity towards complementary DNA and RNA sequences and that as such they are also useful in the design of RNAi trigger structures. Structural studies have shown LNA to be an RNA mimic, fitting seamlessly into an A-type duplex geometry. Several reports have revealed LNA as a most promising molecule for the development of oligonucleotide-based therapeutics.

According to the current disclosure the thermostability provided by LNA's and BNA's allow modifications of underlying RNAi structures. For example, significant truncations in the passenger/sense strand of a given trigger can be made and with the appropriate addition and use of LNA's or BNA's the gene knockdown activity of every shorter passenger strands can be rescued. The result is a LNS enhanced RNAi trigger that is lighter in weight with the equivalent activity in physiological systems. Similarly, phosphorothioate molecules can be used to control or lessen nuclease attack on a given RNAi trigger structure. In this sense the chemical modification of a given RNAi trigger can be a balancing act between those modifications such as LNA, BNA or Phosphorothioate (PS) placement to protect against nuclease degradation or loss of thermostability.

### Lipid-Conjugated RNAi Oligonucleotides

The disclosure provides, inter alia, lipid-conjugated RNAi oligonucleotides (e.g., RNAi oligonucleotide-lipid conjugates) that reduce expression of a target gene. In some embodiments, a lipid-conjugated RNAi oligonucleotide provided by the disclosure is targeted to an mRNA encoding the target gene. Messenger RNA (mRNA) that encodes a target gene and is targeted by a lipid-conjugated RNAi oligonucleotide of the disclosure is referred to herein as “target mRNA”.

mRNA Target Sequences

In some embodiments, the lipid-conjugated RNAi oligonucleotide is targeted to a target sequence comprising a target mRNA. In some embodiments, the lipid-conjugated RNAi oligonucleotide is targeted to a target sequence within a target mRNA. In some embodiments, the lipid-conjugated RNAi oligonucleotide, or a portion, fragment, or strand thereof (e.g., an antisense strand or a guide strand of a double-stranded oligonucleotide) binds or anneals to a target sequence comprising a target mRNA, thereby reducing target gene expression. In some embodiments, the lipid-conjugated RNAi oligonucleotide is targeted to a target sequence comprising target mRNA for the purpose of reducing expression of a target gene in vivo. In some embodiments, the amount or extent of reduction of target gene expression by a lipid-conjugated RNAi oligonucleotide targeted to a specific target sequence correlates with the potency of the lipid-conjugated RNAi oligonucleotide. In some embodiments, the amount or extent of reduction of target gene expression by a lipid-conjugated RNAi oligonucleotide targeted to a specific target sequence correlates with the amount or extent of therapeutic benefit in a subject or patient having a disease, disorder or condition associated with target gene expression treated with the lipid-conjugated RNAi oligonucleotide.

Through examination of the nucleotide sequence of mRNAs encoding target genes, including mRNAs of multiple different species (e.g., human, cynomolgus monkey, mouse, and rat) and as a result of in vitro and in vivo testing, it has been discovered that certain nucleotide sequences and certain systemic modifications to those oligonucleotides are more amenable than others to RNAi oligonucleotide-mediated reduction and are thus useful as part of oligonucleotides that are otherwise targeted to specific gene target sequences. In some embodiments, a sense strand of a lipid-conjugated RNAi oligonucleotide, or a portion or fragment thereof, described herein, comprises a nucleotide sequence that is similar (e.g., having no more than 4 mismatches) or is identical to a target sequence comprising a target mRNA. In some embodiments, a portion or region of the sense strand of a double-stranded oligonucleotide described herein comprises a target sequence comprising a target mRNA.

In some embodiments, the target mRNA is expressed in a tissue or cell of a subject. In some embodiments, the target mRNA is expressed in more than one tissue or cell of a subject, wherein the tissues or cells are different. In some embodiments, the target mRNA is differentially expressed in a tissue or cell. In some embodiments, the target mRNA is expressed throughout multiple tissue types and/or cell types. In some embodiments, the target mRNA is expressed in the central nervous system, liver tissue, ocular tissue, adipose tissue, adrenal tissue, or skeletal muscle tissue. In some embodiments, the target mRNA is expressed in the central nervous system, peripherial nervous system, liver tissue, ocular tissue, adipose tissue, adrenal tissue, heart tissue, lung tissue, skeletal muscle tissue, cardiac muscle tissue, smooth muscle tissue, or kidney tissue, or any combination thereof. In some embodiments, the target mRNA is expressed in the central nervous system. In some embodiments, the target mRNA is expressed in the peripherial nervous system. In some embodiments, the target mRNA is expressed in a region of the central nervous system. In some embodiments, a region of the central nervous system is selected from the eye, brain, cerebrum, cerebral cortex, frontal lobe, frontal cortex, parietal lobe, temporal lobe, occipital lobe, hippocampus, cerebellum, brain stem, dorsal root ganglion (DRG) or the spinal cord. In some embodiments, the target mRNA is expressed in a neuron. In some embodiments, the target mRNA is expressed in a glial cell. In some embodiments, the target mRNA is expressed in a neuron located in the central nervous system. In some embodiments, the target mRNA is expressed in liver tissue. In some embodiments, the target mRNA is expressed in a hepatocyte. In some embodiments, the target mRNA is expressed in a liver sinusoidal endothelial cell. In some embodiments, the target mRNA is expressed in a macrophage. In some embodiments, the target mRNA is expressed in a macrophage located in liver tissue. In some embodiments, the target mRNA is expressed in ocular tissue. In some embodiments, the target mRNA is expressed in the retina and/or optic nerve. In some embodiments, the target mRNA is expressed in adipose tissue. In some embodiments, the target mRNA is expressed in skeletal muscle tissue. In some embodiments, the target mRNA is expressed in adrenal tissue. In some embodiments, the target mRNA is expressed in heart tissue. In some embodiments, the target mRNA is expressed in lung tissue. In some embodiments, the target mRNA is expressed in the eye. In some embodiments, the target mRNA is expressed in the brain. In some embodiments, the target mRNA is expressed in the cerebrum. In some embodiments, the target mRNA is expressed in the cerebellum. In some embodiments, the target mRNA is expressed in the brain stem. In some embodiments, the target mRNA is expressed in the frontal lobe. In some embodiments, the target mRNA is expressed in the frontal cortex. In some embodiments, the target mRNA is expressed in the parietal lobe. In some embodiments, the target mRNA is expressed in the temporal lobe. In some embodiments, the target mRNA is expressed in the occipital lobe. In some embodiments, the target mRNA is expressed in the hippocampus. In some embodiments, the target mRNA is expressed in the DRG. In some embodiments, the target mRNA is expressed in the spinal cord.

### RNAi Oligonucleotide Targeting Sequences

In some embodiments, the lipid-conjugated RNAi oligonucleotides provided by the disclosure comprise a targeting sequence. As used herein, the term “targeting sequence” refers to a nucleotide sequence having a region of complementarity to a specific nucleotide sequence comprising an mRNA. In some embodiments, the lipid-conjugated RNAi oligonucleotides provided by the disclosure comprise a gene targeting sequence having a region of complementarity to a nucleotide sequence comprising a target sequence of a target mRNA.

The targeting sequence imparts the lipid-conjugated RNAi oligonucleotide with the ability to specifically target an mRNA by binding or annealing to a target sequence comprising a target mRNA by complementary (Watson-Crick) base pairing. In some embodiments, the lipid-conjugated RNAi oligonucleotides herein (or a strand thereof, e.g., an antisense strand or a guide strand of a double-stranded oligonucleotide) comprise a targeting sequence having a region of complementarity that binds or anneals to a target sequence comprising a target mRNA by complementary (Watson-Crick) base pairing. In some embodiments, the lipid-conjugated RNAi oligonucleotides herein (or a strand thereof, e.g., an antisense strand or a guide strand of a double-stranded oligonucleotide) comprise a targeting sequence having a region of complementarity that binds or anneals to a target sequence within a target mRNA by complementary (Watson-Crick) base pairing. The targeting sequence is generally of suitable length and base content to enable binding or annealing of the lipid-conjugated RNAi oligonucleotide (or a strand thereof) to a specific target mRNA for purposes of inhibiting target gene expression. In some embodiments, the targeting sequence is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence is at least 12, at least 13, at least 14, at least 15, at least 16, at least 17, at least 18, at least 19 or at least 20 nucleotides. In some embodiments, the targeting sequence is about 12 to about 30 (e.g., 12 to 30, 12 to 22, 15 to 25, 17 to 21, 18 to 27, 19 to 27, or 15 to 30) nucleotides in length. In some embodiments, the targeting sequence is about 12, 13, 14, 15, 16, 17, 18, 19, 20, 21, 22, 23, 24, 25, 26, 27, 28, 29 or 30 nucleotides in length. In some embodiments, the targeting sequence is 18 nucleotides in length. In some embodiments, the targeting sequence is 19 nucleotides in length. In some embodiments, the targeting sequence is 20 nucleotides in length. In some embodiments, the targeting sequence is 21 nucleotides in length. In some embodiments, the targeting sequence is 22 nucleotides in length. In some embodiments, the targeting sequence is 23 nucleotides in length. In some embodiments, the targeting sequence is 24 nucleotides in length.

In some embodiments, the lipid-conjugated RNAi oligonucleotides herein comprise a targeting sequence that is fully complementary to a target sequence comprising a target mRNA. In some embodiments, the lipid-conjugated RNAi oligonucleotides herein comprise a targeting sequence that is fully complementary to a target sequence within a target mRNA. In some embodiments, the targeting sequence is partially complementary to a target sequence comprising a target mRNA. In some embodiments, the targeting sequence is partially complementary to a target sequence within a target mRNA. In some embodiments, the targeting sequence comprises a region of contiguous nucleotides comprising the antisense strand.

In some embodiments, the lipid-conjugated RNAi oligonucleotides herein comprise a targeting sequence that is complementary to a contiguous sequence of nucleotides comprising a target mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the lipid-conjugated RNAi oligonucleotides comprise a targeting sequence that is complementary to a contiguous sequence of nucleotides comprising a target mRNA, wherein the contiguous sequence of nucleotides is 10, 11, 12, 13, 14, 15, 16, 17, 18, 19 or 20 nucleotides in length. In some embodiments, the lipid-conjugated RNAi oligonucleotides comprise a targeting sequence that is complementary to a contiguous sequence of nucleotides comprising a target mRNA, wherein the contiguous sequence of nucleotides is 15 nucleotides in length. In some embodiments, the lipid-conjugated RNAi oligonucleotides comprise a targeting sequence that is complementary to a contiguous sequence of nucleotides comprising a target mRNA, wherein the contiguous sequence of nucleotides is 19 nucleotides in length.

In some embodiments, the lipid-conjugated RNAi oligonucleotide comprises a targeting sequence that is complementary to a contiguous sequence of nucleotides comprising a target mRNA, wherein the contiguous sequence of nucleotides is 15 nucleotides in length. In some embodiments, the lipid-conjugated RNAi oligonucleotide comprises a targeting sequence that is complementary to a contiguous sequence of nucleotides comprising a target mRNA, wherein the contiguous sequence of nucleotides is 19 nucleotides in length.

In some embodiments, a targeting sequence of a lipid-conjugated RNAi oligonucleotide herein is fully complementary (e.g., having no mismatches) to a target sequence comprising a target mRNA and comprises the entire length of an antisense strand. In some embodiments, a targeting sequence of a lipid-conjugated RNAi oligonucleotide herein is fully complementary (e.g., having no mismatches) to a target sequence comprising a target mRNA and comprises a portion of the entire length of an antisense strand. In some embodiments, a targeting sequence of a lipid-conjugated RNAi oligonucleotide herein is fully complementary (e.g., having no mismatches) to a target sequence comprising a target mRNA and comprises 10 to 20 nucleotides of the antisense strand. In some embodiments, a targeting sequence of a lipid-conjugated RNAi oligonucleotide herein is fully complementary (e.g., having no mismatches) to a target sequence comprising a target mRNA and comprises 15 to 19 nucleotides of the antisense strand. In some embodiments, a targeting sequence of a lipid-conjugated RNAi oligonucleotide herein is fully complementary (e.g., having no mismatches) to a target sequence comprising a target mRNA and comprises 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a lipid-conjugated RNAi oligonucleotide herein is fully complementary (e.g., having no mismatches) to a target sequence comprising a target mRNA and comprises 19 nucleotides of the antisense strand.

In some embodiments, a targeting sequence of a lipid-conjugated RNAi oligonucleotide herein is partially complementary (e.g., having no more than 4 mismatches) to a target sequence comprising a target mRNA and comprises the entire length of an antisense strand. In some embodiments, a targeting sequence of a lipid-conjugated RNAi oligonucleotide herein is partially complementary (e.g., having no more than 4 mismatches) to a target sequence comprising a target mRNA and comprises a portion of the entire length of an antisense strand. In some embodiments, a targeting sequence of a lipid-conjugated RNAi oligonucleotide herein is partially complementary (e.g., having no more than 4 mismatches) to a target sequence comprising a target mRNA and comprises 10 to 20 nucleotides of the antisense strand. In some embodiments, a targeting sequence of a lipid-conjugated RNAi oligonucleotide herein is partially complementary (e.g., having no more than 4 mismatches) to a target sequence comprising a target mRNA and comprises 15 to 19 nucleotides of the antisense strand. In some embodiments, a targeting sequence of a lipid-conjugated RNAi oligonucleotide herein is partially complementary (e.g., having no more than 4 mismatches) to a target sequence comprising a target mRNA and comprises 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a lipid-conjugated RNAi oligonucleotide herein is partially complementary (e.g., having no more than 4 mismatches) to a target sequence comprising a target mRNA and comprises 19 nucleotides of the antisense strand.

In some embodiments, a lipid-conjugated RNAi oligonucleotide herein comprises a targeting sequence having one or more base pair (bp) mismatches with the corresponding target sequence comprising a target mRNA. In some embodiments, the targeting sequence has a 1 bp mismatch, a 2 bp mismatch, a 3 bp mismatch, a 4 bp mismatch, or a 5 bp mismatch with the corresponding target sequence comprising a target mRNA provided that the ability of the targeting sequence to bind or anneal to the target sequence under appropriate hybridization conditions and/or the ability of the lipid-conjugated RNAi oligonucleotide to inhibit or reduce target gene expression is maintained (e.g., under physiological conditions). Alternatively, in some embodiments, the targeting sequence comprises no more than 1, no more than 2, no more than 3, no more than 4, or no more than 5 bp mismatches with the corresponding target sequence comprising a target mRNA provided that the ability of the targeting sequence to bind or anneal to the target sequence under appropriate hybridization conditions and/or the ability of the lipid-conjugated RNAi oligonucleotide to inhibit or reduce target gene expression is maintained. In some embodiments, the lipid-conjugated RNAi oligonucleotide comprises a targeting sequence having 1 mismatch with the corresponding target sequence. In some embodiments, the lipid-conjugated RNAi oligonucleotide comprises a targeting sequence having 2 mismatches with the corresponding target sequence. In some embodiments, the lipid-conjugated RNAi oligonucleotide comprises a targeting sequence having 3 mismatches with the corresponding target sequence. In some embodiments, the lipid-conjugated RNAi oligonucleotide comprises a targeting sequence having 4 mismatches with the corresponding target sequence. In some embodiments, the lipid-conjugated RNAi oligonucleotide comprises a targeting sequence having 5 mismatches with the corresponding target sequence. In some embodiments, the lipid-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence. In some embodiments, the lipid-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

### Types of Oligonucleotides

A variety of RNAi oligonucleotide types and/or structures are useful for reducing target gene expression in the methods herein. Any of the RNAi oligonucleotide types described herein or elsewhere are contemplated for use as a framework to incorporate a targeting sequence herein for the purposes of inhibiting or reducing corresponding target gene expression.

In some embodiments, the lipid-conjugated RNAi oligonucleotides herein inhibit target gene expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded extensions (on one or both sides of the molecule) as well as double-stranded extensions.

In some embodiments, the RNAi oligonucleotides conjugates herein engage with the RNAi pathway downstream of the involvement of Dicer (e.g., Dicer cleavage). In some embodiments, the oligonucleotides described herein are Dicer substrates. In some embodiments, the oligonucleotides herein interact with Dicer and are loaded into RISC. In some embodiments, upon endogenous Dicer processing, double-stranded nucleic acids of 19-23 nucleotides in length capable of reducing expression of a target mRNA are produced. In some embodiments, the lipid-conjugated RNAi oligonucleotide has an overhang (e.g., of 1, 2, or 3 nucleotides in length) in the 3′ end of the sense strand. In some embodiments, the lipid-conjugated RNAi oligonucleotide (e.g., siRNA conjugate) comprises a 21-nucleotide guide strand that is antisense to a target mRNA and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621; 9,193,753; 8,420,391; and, 8,552,171 all to Tuschl et al. Such patents also indicate a lack of activity with regard to double overhang constructs.

In some embodiments, the RNAi oligonucleotides conjugates disclosed herein comprise sense and antisense strands that are both in the range of about 17 to 26 (e.g., 17 to 26, 20 to 25 or 21-23) nucleotides in length. In some embodiments, the lipid-conjugated RNAi oligonucleotides disclosed herein comprise a sense and antisense strand that are both in the range of about 19-22 nucleotides in length. In some embodiments, the sense and antisense strands are of equal length. In some embodiments, the lipid-conjugated RNAi oligonucleotides disclosed herein comprise sense and antisense strands, such that there is a 3′-overhang on either the sense strand or the antisense strand, or both the sense and antisense strand. In some embodiments, for lipid-conjugated RNAi oligonucleotides that have sense and antisense strands that are both in the range of about 21-23 nucleotides in length, a 3′ overhang on the sense, antisense, or both sense and antisense strands is 1 or 2 nucleotides in length. In some embodiments, a lipid-conjugated RNAi oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other RNAi oligonucleotide designs for use with the compositions and methods herein include: 16-mer siRNAs (see, e.g., Nucleic Acids in Chemistry and Biology, Blackburn (ed.), ROYAL SOCIETY OF CHEMISTRY, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS Lett. 557:193-98), and small internally segmented interfering RNA (siRNA; see, e.g., Bramsen et al. (2007) NUCLEIC ACIDS RES. 35:5886-97). Further non-limiting examples of an oligonucleotide structure that may be used in some embodiments to reduce or inhibit the expression of a target gene are microRNA (miRNA), short hairpin RNA (shRNA) and short siRNA (see, e.g., Hamilton et al. (2002) EMBO J. 21:4671-79; see also, US Patent Application Publication No. 2009/0099115).

### Antisense Strands

In some embodiments, an antisense strand of a lipid-conjugated RNAi oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, a lipid-conjugated RNAi oligonucleotide herein comprises an antisense strand of up to about 50 nucleotides in length (e.g., up to 50, up to 40, up to 35, up to 30, up to 27, up to 25, up to 21, up to 19, up to 17, up to 15, or up to 8 nucleotides in length). In some embodiments, a lipid-conjugated RNAi oligonucleotide herein comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, a herein comprises an antisense strand in a range of about 8 to about 40 (e.g., 8 to 40, 8 to 36, 8 to 32, 8 to 28, 15 to 40, 15 to 36, 15 to 32, 15 to 30, 15 to 28, 17 to 22, 17 to 25, 19 to 27, 19 to 30, 20 to 40, 22 to 40, 25 to 40 or 32 to 40) nucleotides in length. In some embodiments, a lipid-conjugated RNAi oligonucleotide herein comprises an antisense strand of 15 to 30 nucleotides in length. In some embodiments, a lipid-conjugated RNAi oligonucleotide herein comprises an antisense strand of 12 to 30 nucleotides in length. In some embodiments, an antisense strand of any one of the lipid-conjugated RNAi oligonucleotide disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, a lipid-conjugated RNAi oligonucleotide comprises an antisense strand of 19-23 nucleotides in length. In some embodiments, a lipid-conjugated RNAi oligonucleotide comprises an antisense strand of 19 nucleotides in length. In some embodiments, a lipid-conjugated RNAi oligonucleotide comprises an antisense strand of 20 nucleotides in length. In some embodiments, a lipid-conjugated RNAi oligonucleotide comprises an antisense strand of 21 nucleotides in length. In some embodiments, a lipid-conjugated RNAi oligonucleotide comprises an antisense strand of 22 nucleotides in length. In some embodiments, a lipid-conjugated RNAi oligonucleotide comprises an antisense strand of 23 nucleotides in length.

### Sense Strands

In some embodiments, a lipid-conjugated RNAi oligonucleotide disclosed herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, a lipid-conjugated RNAi oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, a lipid-conjugated RNAi oligonucleotide herein comprises a sense strand of at least about 10 nucleotides in length (e.g., at least 10, at least 12, at least 15, at least 19, at least 21, at least 25, at least 27, at least 30, at least 36 or at least 38 nucleotides in length). In some embodiments, a lipid-conjugated RNAi oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, a lipid-conjugated RNAi oligonucleotide herein comprises a sense strand in a range of about 10 to about 50 (e.g., 10 to 50, 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, a lipid-conjugated RNAi oligonucleotide herein comprises a sense strand 15 to 50 nucleotides in length. In some embodiments, a lipid-conjugated RNAi oligonucleotide herein comprises a sense strand 18 to 38 nucleotides in length. In some embodiments, a lipid-conjugated RNAi oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a lipid-conjugated RNAi oligonucleotide herein comprises a sense strand of 12-21 nucleotides in length. In some embodiments, a lipid-conjugated RNAi oligonucleotide herein comprises a sense strand of 10 nucleotides in length. In some embodiments, a lipid-conjugated RNAi oligonucleotide herein comprises a sense strand of 11 nucleotides in length. In some embodiments, a lipid-conjugated RNAi oligonucleotide herein comprises a sense strand of 12 nucleotides in length. In some embodiments, a lipid-conjugated RNAi oligonucleotide herein comprises a sense strand of 13 nucleotides in length. In some embodiments, a lipid-conjugated RNAi oligonucleotide herein comprises a sense strand of 14 nucleotides in length. In some embodiments, a lipid-conjugated RNAi oligonucleotide herein comprises a sense strand of 15 nucleotides in length. In some embodiments, a lipid-conjugated RNAi oligonucleotide herein comprises a sense strand of 16 nucleotides in length. In some embodiments, a lipid-conjugated RNAi oligonucleotide herein comprises a sense strand of 17 nucleotides in length. In some embodiments, a lipid-conjugated RNAi oligonucleotide herein comprises a sense strand of 18 nucleotides in length. In some embodiments, a lipid-conjugated RNAi oligonucleotide herein comprises a sense strand of 19 nucleotides in length. In some embodiments, a lipid-conjugated RNAi oligonucleotide herein comprises a sense strand of 20 nucleotides in length. In some embodiments, a lipid-conjugated RNAi oligonucleotide herein comprises a sense strand of 21 nucleotides in length. In some embodiments, a lipid-conjugated RNAi oligonucleotide herein comprises a sense strand of 22 nucleotides in length. In some embodiments, a lipid-conjugated RNAi oligonucleotide herein comprises a sense strand of 23 nucleotides in length. In some embodiments, a lipid-conjugated RNAi oligonucleotide herein comprises a sense strand of 24 nucleotides in length. In some embodiments, a lipid-conjugated RNAi oligonucleotide herein comprises a sense strand of 25 nucleotides in length. In some embodiments, a lipid-conjugated RNAi oligonucleotide herein comprises a sense strand of 26 nucleotides in length. In some embodiments, a lipid-conjugated RNAi oligonucleotide herein comprises a sense strand of 27 nucleotides in length. In some embodiments, a lipid-conjugated RNAi oligonucleotide herein comprises a sense strand of 28 nucleotides in length. In some embodiments, a lipid-conjugated RNAi oligonucleotide herein comprises a sense strand of 29 nucleotides in length. In some embodiments, a lipid-conjugated RNAi oligonucleotide herein comprises a sense strand of 30 nucleotides in length. In some embodiments, a lipid-conjugated RNAi oligonucleotide herein comprises a sense strand of 31 nucleotides in length. In some embodiments, a lipid-conjugated RNAi oligonucleotide herein comprises a sense strand of 32 nucleotides in length. In some embodiments, a lipid-conjugated RNAi oligonucleotide herein comprises a sense strand of 33 nucleotides in length. In some embodiments, a lipid-conjugated RNAi oligonucleotide herein comprises a sense strand of 34 nucleotides in length. In some embodiments, a lipid-conjugated RNAi oligonucleotide herein comprises a sense strand of 35 nucleotides in length. In some embodiments, a lipid-conjugated RNAi oligonucleotide herein comprises a sense strand of 36 nucleotides in length. In some embodiments, a lipid-conjugated RNAi oligonucleotide herein comprises a sense strand of 37 nucleotides in length. In some embodiments, a lipid-conjugated RNAi oligonucleotide herein comprises a sense strand of 38 nucleotides in length.

In some embodiments, a sense strand comprises a stem-loop structure at its 3′ end. In some embodiments, a sense strand comprises a stem-loop structure at its 5′ end. In some embodiments, the stem-loop is formed by intrastrand base pairing. In some embodiments, a sense strand comprises a stem-loop structure at its 5′ end. In some embodiments, a stem is a duplex of 1, 2, 3, 4, 5, 6, 7, 8, 9, 10, 11, 12, 13 or 14 nucleotides in length. In some embodiments, the stem of the stem-loop comprises a duplex of 1 nucleotide in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the lipid-conjugated RNAi oligonucleotide protection against degradation (e.g., enzymatic degradation), facilitates or improves targeting and/or delivery to a target cell, tissue, or organ, or both. For example, in some embodiments, the loop of a stem-loop provides nucleotides comprising one or more modifications that facilitate, improve, or increase targeting to a target mRNA (e.g., a target mRNA expressed in the CNS), inhibition of target gene expression, and/or delivery to a target cell, tissue, or organ (e.g., the CNS), or a combination thereof. In some embodiments, the stem-loop itself or modification(s) to the stem-loop do not substantially affect the inherent gene expression inhibition activity of the lipid-conjugated RNAi oligonucleotide, but facilitates, improves, or increases stability (e.g., provides protection against degradation) and/or delivery of the lipid-conjugated RNAi oligonucleotide to a target cell, tissue, or organ (e.g., the CNS). In certain embodiments, a lipid-conjugated RNAi oligonucleotide herein comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length.

In some embodiments, the tetraloop comprises the sequence 5′-GAAA-3′. In some embodiments, the tetraloop comprises the sequence 5′-UNCG-3′. In some embodiments, the tetraloop comprises the sequence 5′-UACG-3′. In some embodiments, the stem loop comprises the sequence 5′-GCAGCCGAAAGGCUGC-3′ (SEQ ID NO: 526).

In some embodiments, a loop (L) of a stem-loop having the structure S1-L-S2 as described above is a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop as described in U.S. Pat. No. 10,131,912, incorporated herein by reference (e.g., within a nicked tetraloop structure).

### Duplex Length

In some embodiments, a duplex formed between a sense and antisense strand is at least 8 (e.g., at least 10, at least 11, at least 12, at least 13, at least 14, at least 15, or at least 16) nucleotides in length. In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is in the range of 10-30 nucleotides in length (e.g., 10 to 30, 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 10, 11, 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 10-18 base pairs in length. In some embodiments, a duplex formed between a sense and antisense strand is 15-30 base pairs in length. In some embodiments, a duplex formed between a sense and antisense strand is 17-21 base pairs in length. In some embodiments, a duplex formed between a sense and antisense strand is 10 base pairs in length. In some embodiments, a duplex formed between a sense and antisense strand is 11 base pairs in length. In some embodiments, a duplex formed between a sense and antisense strand is 12 base pairs in length. In some embodiments, a duplex formed between a sense and antisense strand is 13 base pairs in length. In some embodiments, a duplex formed between a sense and antisense strand is 14 base pairs in length. In some embodiments, a duplex formed between a sense and antisense strand is 15 base pairs in length. In some embodiments, a duplex formed between a sense and antisense strand is 16 base pairs in length. In some embodiments, a duplex formed between a sense and antisense strand is 17 base pairs in length. In some embodiments, a duplex formed between a sense and antisense strand is 18 base pairs in length. In some embodiments, a duplex formed between a sense and antisense strand is 19 base pairs in length. In some embodiments, a duplex formed between a sense and antisense strand is 20 base pairs in length. In some embodiments, a duplex formed between a sense and antisense strand is 21 base pairs in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Ends

In some embodiments, a lipid-conjugated RNAi oligonucleotide disclosed herein comprises sense and antisense strands, such that there is a 3′-overhang on either the sense strand or the antisense strand, or both the sense and antisense strand. In some embodiments, a lipid-conjugated RNAi oligonucleotide herein has one 5′ end that is thermodynamically less stable compared to the other 5′ end. In some embodiments, an asymmetric lipid-conjugated RNAi oligonucleotide conjugate is provided that includes a blunt end at the 3′ end of a sense strand and overhang at the 3′ end of the antisense strand. In some embodiments, a 3′ overhang on an antisense strand is 1-4 nucleotides in length (e.g., 1, 2, 3, or 4 nucleotides in length).

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is about two (2) to about twelve (12) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

Typically, an oligonucleotide for RNAi has a two (2) nucleotide overhang on the 3′ end of the antisense (guide) strand. However, other overhangs are possible. In some embodiments, an overhang is a 3′ overhang comprising a length of between one and four nucleotides, optionally one to four, one to three, one to two, two to four, two to three, or one, two, three, or four nucleotides. In some embodiments, the overhang is a 5′ overhang comprising a length of between one and four nucleotides, optionally one to four, one to three, one to two, two to four, two to three, or one, two, three, or four nucleotides.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, the 5′ overhang is 2 nucleotides and the 3′ overhang is about 3-7 nucleotides. In some embodiments, the 5′ overhang is 2 nucleotides and the 3′ overhang is 3 nucleotides. In some embodiments, the 5′ overhang is 2 nucleotides and the 3′ overhang is 4 nucleotides. In some embodiments, the 5′ overhang is 2 nucleotides and the 3′ overhang is 5 nucleotides. In some embodiments, the 5′ overhang is 2 nucleotides and the 3′ overhang is 6 nucleotides. In some embodiments, the 5′ overhang is 2 nucleotides and the 3′ overhang is 7 nucleotides.

In some embodiments, the 3′ overhang is 6-8 nucleotides and the 5′ overhang is 2-4 nucleotides. In some embodiments, the 3′ overhang is 6 nucleotides and the 5′ overhang is 2 nucleotides. In some embodiments, the 3′ overhang is 6 nucleotides and the 5′ overhang is 3 nucleotides. In some embodiments, the 3′ overhang is 6 nucleotides and the 5′ overhang is 4 nucleotides. In some embodiments, the 3′ overhang is 7 nucleotides and the 5′ overhang is 2 nucleotides. In some embodiments, the 3′ overhang is 7 nucleotides and the 5′ overhang is 3 nucleotides. In some embodiments, the 3′ overhang is 7 nucleotides and the 5′ overhang is 4 nucleotides. In some embodiments, the 3′ overhang is 8 nucleotides and the 5′ overhang is 2 nucleotides. In some embodiments, the 3′ overhang is 8 nucleotides and the 5′ overhang is 3 nucleotides. In some embodiments, the 3′ overhang is 8 nucleotides and the 5′ overhang is 4 nucleotides. In some embodiments, one or more (e.g., 2, 3, or 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, an RNAi oligonucleotide conjugate disclosed herein comprises a stem-loop structure at the 3′ end of the sense strand and comprises two terminal overhang nucleotides at the 3′ end of the antisense strand. In some embodiments, an RNAi oligonucleotide conjugate herein comprises a nicked tetraloop structure, wherein the 3′ end of the sense strand comprises a stem-tetraloop structure and comprises two terminal overhang nucleotides at the 3′ end of the antisense strand.

In some embodiments, an RNAi oligonucleotide conjugate disclosed herein comprises a stem-loop structure at the 5′ end of the sense strand and comprises an overhang nucleotides at the 3′ end of the antisense strand. In some embodiments, an RNAi oligonucleotide conjugate herein comprises a nicked tetraloop structure, wherein the 5′ end of the sense strand comprises a stem-tetraloop structure and comprises two terminal overhang nucleotides at the 3′ end of the antisense strand.

In some embodiments, an RNAi oligonucleotide conjugate disclosed herein comprises a stem-loop structure at the 5′ end of the sense strand and comprises a blunt end at the 5′ end of the antisense strand.

In some embodiments, an RNAi oligonucleotide conjugate disclosed herein comprises an overhang of 1-8 nucleotides at the 5′ end of the sense strand and comprises an overhang of 1-8 nucleotides at the 5′ end of the antisense strand.

In some embodiments, the overhang is selected from AA, GG, AG, and GA. In some embodiments, the overhang is AA. In some embodiments, the overhang is AG. In some embodiments, the overhang is GA. In some embodiments, the two terminal overhang nucleotides are GG. Typically, one or both of the two terminal GG nucleotides of the antisense strand are not complementary with the target.

In some embodiments, the 5′ end and/or the 3′ end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

In some embodiments, the sense strand is 18 nucleotides, and the antisense strand comprises a 5′ overhang of 2 nucleotides, and a 3′ overhang of 2 nucleotides. In some embodiments, the sense strand is 17 nucleotides, and the antisense strand comprises a 5′ overhang of 3 nucleotides, and a 3′ overhang of 2 nucleotides. In some embodiments, the sense strand is 16 nucleotides, and the antisense strand comprises a 5′ overhang of 4 nucleotides, and a 3′ overhang of 2 nucleotides. In some embodiments, the sense strand is 13 nucleotides, and the antisense strand comprises a 5′ overhang of 2 nucleotides, and a 3′ overhang of 7 nucleotides. In some embodiments, the sense strand is 12 nucleotides, and the antisense strand comprises a 5′ overhang of 2 nucleotides, and a 3′ overhang of 8 nucleotides. In some embodiments, the sense strand is 12 nucleotides, and the antisense strand comprises a 5′ overhang of 3 nucleotides, and a 3′ overhang of 7 nucleotides. In some embodiments, the sense strand is 10 nucleotides, and the antisense strand comprises a 5′ overhang of 1 nucleotide, and a 3′ overhang of 11 nucleotides.

In some embodiments, the sense strand is 18 nucleotides, the duplex region is 18 nucleotides, and the antisense strand comprises a 5′ overhang of 2 nucleotides, and a 3′ overhang of 2 nucleotides. In some embodiments, the sense strand is 17 nucleotides, the duplex region is 17 nucleotides, and the antisense strand comprises a 5′ overhang of 3 nucleotides, and a 3′ overhang of 2 nucleotides. In some embodiments, the sense strand is 16 nucleotides, the duplex region is 16 nucleotides, and the antisense strand comprises a 5′ overhang of 4 nucleotides, and a 3′ overhang of 2 nucleotides. In some embodiments, the sense strand is 13 nucleotides, the duplex region is 13 nucleotides, and the antisense strand comprises a 5′ overhang of 2 nucleotides, and a 3′ overhang of 7 nucleotides. In some embodiments, the sense strand is 12 nucleotides, the duplex region is 12 nucleotides, and the antisense strand comprises a 5′ overhang of 2 nucleotides, and a 3′ overhang of 8 nucleotides. In some embodiments, the sense strand is 12 nucleotides, the duplex region is 12 nucleotides, and the antisense strand comprises a 5′ overhang of 3 nucleotides, and a 3′ overhang of 7 nucleotides. In some embodiments, the sense strand is 10 nucleotides, the duplex region is 10 nucleotides, and the antisense strand comprises a 5′ overhang of 1 nucleotide, and a 3′ overhang of 11 nucleotides.

### Oligonucleotide Modifications

In some embodiments, an RNAi oligonucleotide conjugate disclosed herein comprises one or more modifications. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. In some embodiments, the sugar moiety of all nucleotides comprising the oligonucleotide is modified at the 2′ position, except for the nucleotide conjugated to a lipid (e.g., the 5′-terminal nucleotide of the sense strand). The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a lipid-conjugated RNAi oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the lipid-conjugated RNAi oligonucleotide comprises at least about 1 modified nucleotide (e.g., at least 1, at least 5, at least 10, at least 15, at least 20, at least 25, at least 30, at least 35, or more). In some embodiments, the antisense strand of the lipid-conjugated RNAi oligonucleotide comprises at least about 1 modified nucleotide (e.g., at least 1, at least 5, at least 10, at least 15, at least 20, or more).

In some embodiments, all the nucleotides of the sense strand of the lipid-conjugated RNAi oligonucleotide are modified. In some embodiments, all the nucleotides of the antisense strand of the lipid-conjugated RNAi oligonucleotide are modified. In some embodiments, all the nucleotides of the lipid-conjugated RNAi oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides lipid-conjugated RNAi oligonucleotides having different modification patterns. In some embodiments, the modified lipid-conjugated RNAi oligonucleotides comprise a sense strand sequence having a modification pattern as set forth in the Examples and Sequence Listing and an antisense strand having a modification pattern as set forth in the Examples and Sequence Listing.

In some embodiments, a lipid-conjugated RNAi oligonucleotide disclosed herein comprises an antisense strand having nucleotides that are modified with 2′-F. In some embodiments, a lipid-conjugated RNAi oligonucleotide disclosed herein comprises an antisense strand comprises nucleotides that are modified with 2′-F and 2′-OMe. In some embodiments, a lipid-conjugated RNAi oligonucleotide disclosed herein comprises a sense strand having nucleotides that are modified with 2′-F. In some embodiments, a lipid-conjugated RNAi oligonucleotide disclosed herein comprises a sense strand comprising nucleotides that are modified with 2′-F and 2′-OMe. In some embodiments, a lipid-conjugated RNAi oligonucleotide disclosed herein comprises a sense strand comprising nucleotides that are modified with 2′-F and 2′-OMe, provided that a nucleotide conjugated to a lipid moiety is not modified with 2′-F or 2′-OMe.

In some embodiments, an oligonucleotide described herein comprises a sense strand with about 10-25%, 10%, 11%, 12%, 13%, 14% 15%, 16%, 17%, 18%, 19% or 20% of the nucleotides of the sense strand comprising a 2′-fluoro modification. In some embodiments, about 11% of the nucleotides of the sense strand comprise a 2-fluoro modification. In some embodiments, about 20% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification. In some embodiments, about 26% of the nucleotides in the oligonucleotide comprise a 2′-fluoro modification.

In some embodiments, one or more of positions 8, 9, 10 or 11 of the sense strand is modified with a 2′-F group. In some embodiments, one or more nucleotides forming a base pair with a nucleotide at one or more of positions 10-13 of the antisense strand, is modified with a 2′-F group. In some embodiments, the sugar moiety at each of nucleotides not modified with a 2′-F group or conjugated to a lipid in the sense strand is modified with a 2′-OMe. In some embodiments, the sugar moiety at each of nucleotides at positions 1-7 and 12-20 in the sense strand is modified with a 2′-OMe.

In some embodiments, a lipid-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at one or more of positions 10, 11, 12, and 13 of the antisense strand. In some embodiments, a lipid-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a lipid-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, and 13 of the antisense strand.

In some embodiments, the sense strand comprises at least one 2′-F modified nucleotide wherein the remaining nucleotides not modified with a 2′-F group or conjugated to a lipid are modified with a 2′-OMe.

In some embodiments, the antisense strand has 7 nucleotides that are modified at the 2′ position of the sugar moiety with a 2′-F. In some embodiments, the sugar moiety at positions 2, 3, 4, 5, 7, 10, and 14 of the antisense strand are modified with a 2′-F. In some embodiments, the antisense strand has 14 nucleotides that are modified at the 2′ position of the sugar moiety with a 2′-OMe. In some embodiments, the sugar moiety at positions 6, 8, 9, 11, 12, 13, 15, 16, 17, 18, 19, 20, 21, and 22 of the antisense strand are modified with a 2′-OMe.

In some embodiments, the sense strand has 4 nucleotides that are modified at the 2′ position of the sugar moiety with a 2′-F. In some embodiments, the sugar moiety at positions 2, 3, 8, 9, 10, and 11 of the sense strand are modified with a 2′-F. In some embodiments, the sense strand has 15 nucleotides that are modified at the 2′ position of the sugar moiety with a 2′-OMe. In some embodiments, the sugar moiety at positions 6, 8, 9, 11, 12, 13, 15, 16, 17, 18, 19, 20, 21, and 22 of the antisense strand are modified with a 2′-OMe.

In some embodiments, the sense strand comprises a 2′-fluoro modification at positions 3-6 or 4-7, numbered 5′ to 3′. In some embodiments, the sense strand comprises a 2′-fluoro modification at positions 3-6. In some embodiments, the sense strand comprises a 2′-fluoro modification at positions 4-7.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In some embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In other embodiments, the sugar moiety at each of the positions at positions 2, 4, 5 and 14 of the antisense strand is modified with the 2′-F. In some embodiments, the sugar moiety at each of the positions at positions 1, 2, 3, 5, 7 and 14 of the antisense strand is modified with the 2′-F. In some embodiments, the sugar moiety at each of the positions at positions 2, 3, 4, 5, 7 and 14 of the antisense strand is modified with the 2′-F. In some embodiments, the sugar moiety at each of the positions at positions 1, 2, 3, 5, 10 and 14 of the antisense strand is modified with the 2′-F. In some embodiments, the sugar moiety at each of the positions at positions 2, 3, 4, 5, 10 and 14 of the antisense strand is modified with the 2′-F. In some embodiments, the sugar moiety at each of the positions at positions 2, 3, 5, 7, 10 and 14 of the antisense strand is modified with the 2′-F. In some embodiments, the sugar moiety at each of the positions at positions 2, 3, 4, 5, 7, 10 and 14 of the antisense strand is modified with the 2′-F. In some embodiments, the antisense strand has 9 nucleotides that are modified at the 2′-position of the sugar moiety with a 2′-F. In some embodiments, the sugar moiety at each of the positions at positions 2, 3, 4, 5, 7, 10, 14, 16 and 19 of the antisense strand is modified with the 2′-F.

In some embodiments, a lipid-conjugated RNAi oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, a lipid-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises a sense strand having the sugar moiety at positions 8-11 modified with 2′-F. In some embodiments, a lipid-conjugated RNAi oligonucleotide provided herein comprises a sense strand having the sugar moiety at positions 3, 5, 8, 10, 12, 13, 15 and 17 modified with 2′-F. In some embodiments, a lipid-conjugated RNAi oligonucleotide provided herein comprises a sense strand having the sugar moiety at positions 1-7 and 12-17 or 12-20 modified with 2′OMe. In some embodiments, a lipid-conjugated RNAi oligonucleotide provided herein comprises a sense strand having the sugar moiety at positions 2-7 and 12-17 or 12-20 modified with 2′OMe. In some embodiments, a lipid-conjugated RNAi oligonucleotide provided herein comprises a sense strand having the sugar moiety at positions 1-6 and 12-17 or 12-20 modified with 2′OMe. In some embodiments, a lipid-conjugated RNAi oligonucleotide provided herein comprises a sense strand having the sugar moiety at positions 1, 2, 4, 6, 7, 9, 11, 14, 16 and 18-20 modified with 2′OMe. In some embodiments, a lipid-conjugated RNAi oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety of each of the nucleotides at positions 2-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety of each of the nucleotides at positions 1-6 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, a lipid-conjugated RNAi oligonucleotide provided herein comprises a sense strand having the sugar moiety at positions 1, 2, 4, 6, 7, 9, 11, 14, 16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37 or position 38 modified with 2′-F.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37 or position 38 modified with 2′-OMe.

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a lipid-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37 or position 38 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, a lipid-conjugated RNAi oligonucleotide described herein comprises a 5′-terminal phosphate. In some embodiments, the 5′-terminal phosphate groups of the lipid-conjugated RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, a lipid-conjugated RNAi oligonucleotide herein comprises analogs of 5′ phosphates that are resistant to such degradation. In some embodiments, the phosphate analog is oxymethyl phosphonate, vinyl phosphonate or malonyl phosphonate, or a combination thereof. In some embodiments, the 5′ end of a lipid-conjugated RNAi oligonucleotide strand is attached to chemical moiety that mimics the electrostatic and steric properties of a natural 5′-phosphate group (“phosphate mimic”).

In some embodiments, a lipid-conjugated RNAi oligonucleotide herein has a phosphate analog at a 4′-carbon position of the sugar (referred to as a “4′-phosphate analog”). See, e.g., Intl. Patent Application Publication No. WO 2018/045317. In some embodiments, a lipid-conjugated RNAi oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethyl phosphonate or an aminomethyl phosphonate, in which the sulfur atom of the thiomethyl group or the nitrogen atom of the amino methyl group is bound to the 4′-carbon of the sugar moiety or analog thereof. In some embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In some embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy. In some embodiments, the 4′-phosphate analog is 4′-oxymethyl phosphonate.

In some embodiments, a lipid-conjugated RNAi oligonucleotide provided herein comprises an antisense strand comprising a 4′-phosphate analog at the 5′-terminal nucleotide, wherein 5′-terminal nucleotide comprises the following structure:

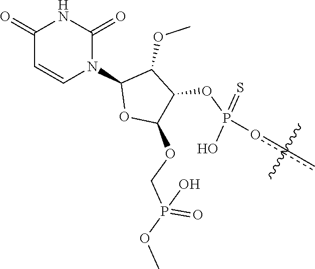

4′-O-monomethylphosphonate-2′-O-methyluridine phosphorothioate [MePhosphonate-4O-mUs, alternatively referred to as “MeMOP”]

### Modified Internucleotide Linkage

In some embodiments, a lipid-conjugated RNAi oligonucleotide herein comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, a lipid-conjugated RNAi oligonucleotide provided herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 18 and 19 of the sense strand, positions 19 and 20 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, the oligonucleotide described herein has a phosphorothioate linkage between each of positions 1 and 2 of the sense strand, the third to last position and penultimate position of the sense strand, and the penultimate position and ultimate position of the sense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 13 and 14 of the antisense strand, positions 14 and 15 of the antisense strand, positions 20 and 21 of the antisense strand, and positions 21 and 22 of the antisense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 13 and 14 of the antisense strand, positions 14 and 15 of the antisense strand, positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand.

In some embodiments, the oligonucleotide comprises phosphorothioate linkages on the sense strand between nucleotides at positions 1 and 2, 8 and 9, and 9 and 10. In some embodiments, the oligonucleotide comprises phosphorothioate linkages on the antisense strand between nucleotides at positions 1 and 2, 2 and 3, 3 and 4, 12 and 14, 14 and 15, 20 and 21, and 21 and 22.

In some embodiments, the oligonucleotide described herein has a phosphorothioate linkage between positions 1 and 2 of the sense strand, positions 1 and 2 of the antisense strand, positions 2 and 3 of the antisense strand, positions 3 and 4 of the antisense strand, positions 12 and 13 of the antisense strand, positions 13 and 14 of the antisense strand, positions 14 and 15 of the antisense strand, positions 15 and 16 of the antisense strand. positions 16 and 17 of the antisense strand, positions 17 and 18 of the antisense strand, positions 18 and 19 of the antisense strand, positions 19 and 20 of the antisense strand, positions 20 and 21 of the antisense strand, and positions 21 and 22 of the antisense strand. In some embodiments, the oligonucleotide comprises a nucleotide at position 14 of a 22 nucleotide antisense strand, wherein the nucleotide is flanked by phosphorothioate linkages (i.e. a phosphorothioate linkage between positions 13 and 14 and between positions 14 and 15). In some embodiments, the flanked nucleotide at position 14 is the ultimate nucleotide of a duplex between the antisense strand and sense strand. In some embodiments, the oligonucleotide comprises a sense and antisense strand, wherein the antisense strand comprises a flanked oligonucleotide at position 14 of a 22 nucleotide antisense strand (i.e. a phosphorothioate linkage between positions 13 and 14 and between positions 14 and 15), wherein the sense and antisense strand form a duplex and the antisense strand comprises an overhang, and wherein the nucleotide at position 14 is within the overhang.

In some embodiments, an oligonucleotide conjugate described herein comprises a peptide nucleic acid (PNA). PNAs are oligonucleotide mimics in which the sugar-phosphate backbone has been replaced by a pseudopeptide skeleton, composed of N-(2-aminoethyl)glycine units. Nucleobases are linked to this skeleton through a two-atom carboxymethyl spacer. In some embodiments, an oligonucleotide conjugate described herein comprises a morpholino oligomer (PMO) comprising an internucleotide linkage backbone of methylene morpholine rings linked through phosphorodiamidate groups.

### Base Modifications

In some embodiments, a lipid-conjugated RNAi oligonucleotide herein comprises one or more modified nucleobases. In some embodiments, modified nucleobases (also referred to herein as base analogs) are linked at the 1′ position of a nucleotide sugar moiety. In some embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tm than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tm than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

### Tm-Increasing Nucleotides

In some embodiments, the oligonucleotide described herein comprises at least one Tm-increasing nucleotide in the sense strand. In some embodiments, the oligonucleotide has one Tm-increasing nucleotide in the sense strand. In some embodiments, the oligonucleotide has up to two Tm-increasing nucleotides in the sense strand. In some embodiments, the oligonucleotide has up to three Tm-increasing nucleotides in the sense strand. In some embodiments, the oligonucleotide has up to four Tm-increasing nucleotides in the sense strand. In some embodiments, the oligonucleotide has up to five Tm-increasing nucleotides in the sense strand. In some embodiments, the oligonucleotide has up to six Tm-increasing nucleotides in the sense strand. In some embodiments, the oligonucleotide has up to seven Tm-increasing nucleotides in the sense strand. In some embodiments, the oligonucleotide has up to eight Tm-increasing nucleotides in the sense strand. In some embodiments, the oligonucleotide has up to nine Tm-increasing nucleotides in the sense strand. In some embodiments, the oligonucleotide has up to ten Tm-increasing nucleotides in the sense strand.

In some embodiments, the oligonucleotide has 1 to 2 Tm-increasing nucleotides in the sense strand. In some embodiments, the oligonucleotide has 1 to 3 Tm-increasing nucleotides in the sense strand. In some embodiments, the oligonucleotide has 1 to 4 Tm-increasing nucleotides in the sense strand. In some embodiments, the oligonucleotide has 1 to 5 Tm-increasing nucleotides in the sense strand.

In some embodiments, an oligonucleotide comprising a stem-loop comprises a Tm-increasing nucleotide in the stem. In some embodiments, an oligonucleotide comprising a stem-loop comprises Tm-increasing nucleotides in at least one base pair of the stem. In some embodiments, an oligonucleotide comprising a stem-loop comprises Tm-increasing nucleotides in one base pair of the stem. In some embodiments, an oligonucleotide comprising a stem-loop comprises Tm-increasing nucleotides in two base pairs of the stem. In some embodiments, an oligonucleotide comprising a stem-loop comprises Tm-increasing nucleotides in three base pairs of the stem. In some embodiments, an oligonucleotide comprising a stem-loop comprises Tm-increasing nucleotides in four base pairs of the stem. In some embodiments, an oligonucleotide comprising a stem-loop comprises Tm-increasing nucleotides in five base pairs of the stem. In some embodiments, an oligonucleotide comprising a stem-loop comprises Tm-increasing nucleotides in six base pairs of the stem.

Tₘ-increasing nucleotides include, but are not limited to, bicyclic nucleotides, tricyclic nucleotides, a G-clamp, and analogues thereof, hexitol nucleotides, or a modified nucleotide. In some embodiments, the Tm-increasing nucleotide is a bicyclic nucleotide. In some embodiments, the Tm-increasing nucleotide is a locked nucleic acid (LNA).

In some embodiments, the sense strand of the oligonucleotide comprises a Tm-increasing nucleotide at one or more of positions 2, 7, 8, 9, 10, 11, 12, 14, 15, 16, 18, and 19. In some embodiments, the sense strand of the oligonucleotide comprises a Tm-increasing nucleotide at position 2. In some embodiments, the sense strand of the oligonucleotide comprises a Tm-increasing nucleotide at position 9. In some embodiments, the sense strand of the oligonucleotide comprises a Tm-increasing nucleotide at position 10. In some embodiments, the sense strand of the oligonucleotide comprises a Tm-increasing nucleotide at position 11. In some embodiments, the sense strand of the oligonucleotide comprises a Tm-increasing nucleotide at position 12. In some embodiments, the sense strand of the oligonucleotide comprises a Tm-increasing nucleotide at position 14. In some embodiments, the sense strand of the oligonucleotide comprises a Tm-increasing nucleotide at position 15. In some embodiments, the sense strand of the oligonucleotide comprises a Tm-increasing nucleotide at position 16. In some embodiments, the sense strand of the oligonucleotide comprises a Tm-increasing nucleotide at position 18. In some embodiments, the sense strand of the oligonucleotide comprises a Tm-increasing nucleotide at position 19.

In some embodiments, a 10-nucleotide sense strand, with nucleotides numbered 5′ to 3′, comprises a Tm-increasing nucleotide at one or more of positions 2, 6 and 7. In some embodiments, a 10-nucleotide sense strand, with nucleotides numbered 5′ to 3′, comprises a Tm-increasing nucleotide at positions 2. In some embodiments, a 10-nucleotide sense strand, with nucleotides numbered 5′ to 3′, comprises a Tm-increasing nucleotide at positions 2 and 6. In some embodiments, a 10-nucleotide sense strand, with nucleotides numbered 5′ to 3′, comprises a Tm-increasing nucleotide at positions 2 and 7. In some embodiments, a 10-nucleotide sense strand, with nucleotides numbered 5′ to 3′, comprises a Tm-increasing nucleotide at 6 and 7. In some embodiments, a 10-nucleotide sense strand, with nucleotides numbered 5′ to 3′, comprises a Tm-increasing nucleotide at positions 2, 6 and 7.

In some embodiments, a 12-nucleotide sense strand, with nucleotides numbered 5′ to 3′, comprises a Tm-increasing nucleotide at one or more of positions 2, 7, 8, 10, and 11. In some embodiments, a 12-nucleotide sense strand, with nucleotides numbered 5′ to 3′, comprises a Tm-increasing nucleotide at position 2. In some embodiments, a 12-nucleotide sense strand, with nucleotides numbered 5′ to 3′, comprises a Tm-increasing nucleotide at position 2 and position 7. In some embodiments, a 12-nucleotide sense strand, with nucleotides numbered 5′ to 3′, comprises a Tm-increasing nucleotide at position 2, position 7, and position 8. In some embodiments, a 12-nucleotide sense strand, with nucleotides numbered 5′ to 3′, comprises a Tm-increasing nucleotide at position 2, position 7, position 8, and position 10. In some embodiments, a 12-nucleotide sense strand, with nucleotides numbered 5′ to 3′, comprises a Tm-increasing nucleotide at position 2, position 7, position 8, position 10, and position 11. In some embodiments, a 12-nucleotide sense strand, with nucleotides numbered 5′ to 3′, comprises a Tm-increasing nucleotide at position 2, position 10, and position 11. In some embodiments, the sense strand comprises a Tm-increasing nucleotide at position 2, position 11, and position 12.

In some embodiments, a 14-nucleotide sense strand, with nucleotides numbered 5′ to 3′, comprises a Tm-increasing nucleotide at one or more of positions 2, 9, 10, 12, and 13. In some embodiments, a 14-nucleotide sense strand, with nucleotides numbered 5′ to 3′, comprises a Tm-increasing nucleotide at position 2. In some embodiments, a 14-nucleotide sense strand, with nucleotides numbered 5′ to 3′, comprises a Tm-increasing nucleotide at position 2 and position 9. In some embodiments, a 14-nucleotide sense strand, with nucleotides numbered 5′ to 3′, comprises a Tm-increasing nucleotide at position 2, position 9, and position 10. In some embodiments, a 14-nucleotide sense strand, with nucleotides numbered 5′ to 3′, comprises a Tm-increasing nucleotide at position 2, position 9, position 10, position 12, and position 13.

In some embodiments, a 16-nucleotide sense strand, with nucleotides numbered 5′ to 3′, comprises a Tm-increasing nucleotide at one or more of positions 2, 11, 12, 14, and 15. In some embodiments, a 16-nucleotide sense strand, with nucleotides numbered 5′ to 3′, comprises a Tm-increasing nucleotide at position 2. In some embodiments, a 16-nucleotide sense strand, with nucleotides numbered 5′ to 3′, comprises a Tm-increasing nucleotide at position 2 and position 11. In some embodiments, a 16-nucleotide sense strand, with nucleotides numbered 5′ to 3′, comprises a Tm-increasing nucleotide at position 2, position 11, and position 12. In some embodiments, a 16-nucleotide sense strand, with nucleotides numbered 5′ to 3′, comprises a Tm-increasing nucleotide at position 2, position 11, position 12, and position 14. In some embodiments, a 16-nucleotide sense strand, with nucleotides numbered 5′ to 3′, comprises a Tm-increasing nucleotide at position 2, position 11, position 12, position 14, and position 15.

In some embodiments, a 20-nucleotide sense strand, with nucleotides numbered 5′ to 3′, comprises a Tm-increasing nucleotide at one or more of positions 2, 15, 16, 18, and 19. In some embodiments, a 20-nucleotide sense strand, with nucleotides numbered 5′ to 3′, comprises a Tm-increasing nucleotide at position 2. In some embodiments, a 20-nucleotide sense strand, with nucleotides numbered 5′ to 3′, comprises a Tm-increasing nucleotide at position 2 and position 15. In some embodiments, a 20-nucleotide sense strand, with nucleotides numbered 5′ to 3′, comprises a Tm-increasing nucleotide at position 2, position 15, and position 16. In some embodiments, a 20-nucleotide sense strand, with nucleotides numbered 5′ to 3′, comprises a Tm-increasing nucleotide at position 2, position 15, position 16, and position 18. In some embodiments, a 20-nucleotide sense strand, with nucleotides numbered 5′ to 3′, comprises a Tm-increasing nucleotide at position 2, position 15, position 16, position 18, and position 19.

In some embodiments, the disclosure provides an RNAi oligonucleotide for reducing target gene expression by the RNAi pathway comprising a combination of one or more Tm-increasing nucleotides and one or more nucleotides (e.g., a modified nucleotide) having a lower binding affinity, wherein the duplex region comprising the RNAi oligonucleotide is maintained under physiological conditions and the ability of the RNAi oligonucleotide to inhibit or reduce target gene expression is maintained.

### Bicyclic Nucleotides

Bicyclic nucleotides typically have a sugar moiety with a 4 to 7 membered ring (including but not limited to a furanosyl) comprising a bridge connecting two atoms of the 4 to 7 membered ring to form a second ring, resulting in a bicyclic structure. Such bicyclic nucleotides have various names including BNA's and LNA's for bicyclic nucleic acids and locked nucleic acids, respectively. The synthesis of bicyclic nucleotides and their incorporation into nucleic acid compounds has also been reported in the literature, including, for example, Singh et al., Chem. Commun., 1998, 4, 455-56; Koshkin et al., TETRAHEDRON, 1998, 54, 3607-30; Wahlestedt et al., PROC. NATL. ACAD. SCI. U.S.A., 2000, 97, 5633-38; Kumar et al., BIOORG. MED. CHEM. LETT., 1998, 8, 2219-22; Singh et al., J. ORG. CHEM., 1998, 63, 10035-039; U.S. Pat. Nos. 7,427,672, 7,053,207, 6,794,499, 6,770,748, 6,268,490 and 6,794,499; and published U.S. applications 20040219565, 20040014959, 20030207841, 20040192918, 20030224377, 20040143114 and 20030082807; each of which is incorporated by reference herein, in its entirety.

In some embodiments, the Tₘ-increasing nucleotide is a bicyclic nucleotide that comprises a bicyclic sugar moiety. In certain embodiments, the bicyclic sugar moiety comprises a first ring of 4 to 7 members and a bridge forming a North-type sugar confirmation that connects any two atoms of the first ring of the sugar moiety to form a second ring. In certain embodiments, the bridge connects the 2′-carbon and the 4′-carbon of the first ring to form a second ring.

Typically, the bridge contains 2 to 8 atoms. In certain embodiments, the bridge contains 3 atoms. In certain embodiments, the bridge contains 4 atoms. In certain embodiments, the bridge contains 5 atoms. In certain embodiments, the bridge contains 6 atoms. In certain embodiments, the bridge contains 7 atoms. In certain embodiments, the bridge contains 8 atoms. In certain embodiments, the bridge contains more than 8 atoms.

In certain embodiments, the bicyclic sugar moiety is a substituted furanosyl comprising a bridge that connects the 2′-carbon and the 4′-carbon of the furanosyl to form the second ring. In certain embodiments, the bicyclic nucleotide has the structure of Formula I:

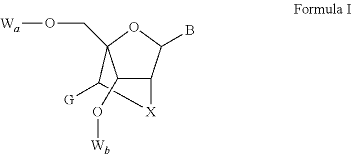

wherein B is a nucleobase;
wherein G is H, OH, NH₂, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio;
wherein X is O, S, or NR₁, wherein R₁ is H, C₁-C₆ alkyl, C₁-C₆ alkoxy, benzene or pyrene; and
wherein Wₐ and W_{b} are each independently, H, OH, a hydroxyl protecting group, a phosphorous moiety, or an internucleotide linking group attaching the nucleotide represented by Formula I to another nucleotide or to an oligonucleotide and wherein at least one of Wₐᵣ or W_{b} is an internucleotide linking group attaching the nucleotide represented by Formula I to an oligonucleotide.

In certain embodiments of Formula I, G is H and X is NR₁, wherein R₁ is benzene or pyrene. In certain embodiments, of Formula I, G is H and X is S.

In certain embodiments of Formula I, G is H and X is O:

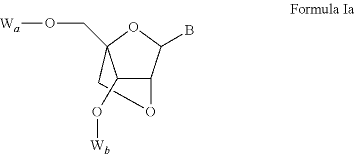

In certain embodiments of Formula I, G is H and X is NR₁, wherein R₁ is H, CH₃, or OCH₃:

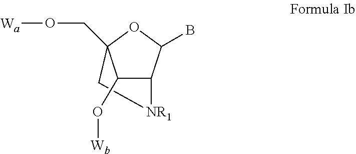

In certain embodiments of Formula I, G is OH or NH₂ and X is O.

In certain embodiments of Formula I, G is OH and X is O:

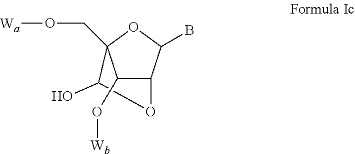

In certain embodiments of Formula I, G is NH₂ and X is O:

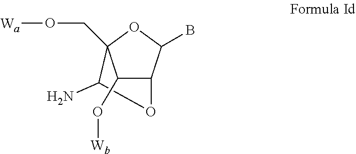

In certain embodiments, of Formula I, G is CH₃ or CH₂OCH₃ and X is O. In certain embodiments, of Formula I, G is CH₃ and X is O:

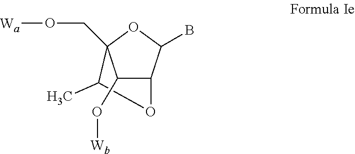

In certain embodiments, of Formula I, G is CH₂OCH₃ and X is O:

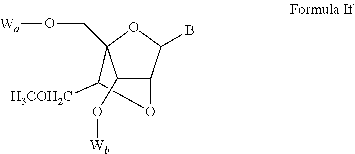

In certain embodiments, the bicyclic nucleotide has the structure of Formula II:

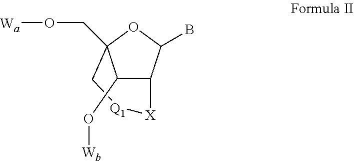

wherein B is a nucleobase;
   wherein Q₁ is CH₂ or O;
   wherein X is CH₂, O, S, or NR₁, wherein R₁ is H, C₁-C₆ alkyl, C₁-C₆ alkoxy, benzene or pyrene;
   wherein if Q₁ is O, X is CH₂;
   wherein if Q₁ is CH₂, X is CH₂, O, S, or NR₁, wherein R₁ is H, C₁-C₆ alkyl, C₁-C₆ alkoxy, benzene or pyrene;
   wherein Wₐ and W_{b} are each independently, H, OH, a hydroxyl protecting group, a phosphorous moiety, or an internucleotide linking group attaching the nucleotide represented by Formula II to another nucleotide or to an oligonucleotide and wherein at least one of Wₐ or W_{b} is an internucleotide linking group attaching the nucleotide represented by Formula II to an oligonucleotide.

In certain embodiments of Formula II, Q₁ is O and X is CH₂:

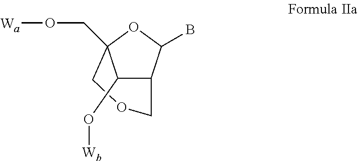

In certain embodiments of Formula II, Q₁ is CH₂ and X is O:

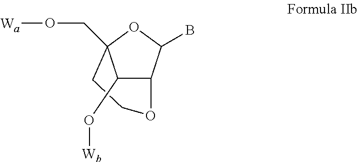

In certain embodiments of Formula II, Q₁ is CH₂ and X is NR₁, wherein R₁ is H, CH₃ or OCH₃:

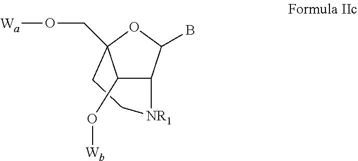

In certain embodiments of Formula II, Q₁ is CH₂ and X is NH:

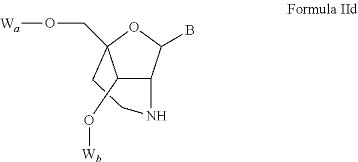

In certain embodiments, the bicyclic nucleotide has the structure of Formula II:

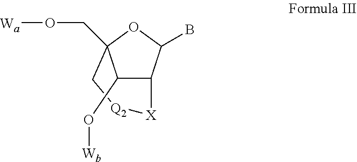

wherein B is a nucleobase;
wherein Q₂ is O or NR₁, wherein R₁ is H, C₁-C₆ alkyl, C₁-C₆ alkoxy, benzene or pyrene;
wherein X is CH₂, O, S, or NR₁, wherein R₁ is H, C₁-C₆ alkyl, C₁-C₆ alkoxy, benzene or pyrene;
wherein if Q₂ is O, X is NR₁;
wherein if Q₂ is NR₁, X is O or S;
wherein Wₐ and W_{b} are each independently, H, OH, a hydroxyl protecting group, a phosphorous moiety, or an internucleotide linking group attaching the nucleotide represented by Formula III to another nucleotide or to an oligonucleotide and wherein at least one of Wₐ or W_{b} is an internucleotide linking group attaching the nucleotide represented by Formula III to an oligonucleotide.

In certain embodiments of Formula III, Q₂ is O and X is NR₁. In certain embodiments of Formula III, Q₂ is O and X is NR₁, wherein R₁ is C₁-C₆ alkyl. In certain embodiments of Formula III, Q₂ is O and X is NR₁ and R₁ is H or CH₃.

In certain embodiments of Formula III, Q₂ is O and X is NR₁ and R₁ is CH₃:

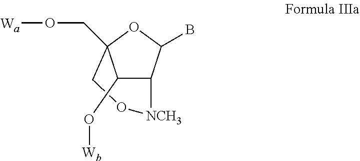

In certain embodiments of Formula III, Q₂ is NR₁ and X is O. In certain embodiments of Formula III, Q₂ is NR₁, wherein R₁ is C₁-C₆ alkyl and X is O.

In certain embodiments of Formula III, Q₂ is NCH₃ and X is O:

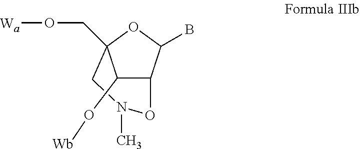

In certain embodiments, the bicyclic nucleotide has the structure of Formula IV:

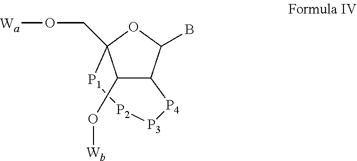

wherein B is a nucleobase;
wherein P₁ and P₃ are CH₂, P₂ is CH₂ or O and P₄ is O; and
wherein Wₐ and W_{b} are each independently, H, OH, a hydroxyl protecting group, a phosphorous moiety, or an internucleotide linking group attaching the nucleotide represented by Formula IV to another nucleotide or to an oligonucleotide and wherein at least one of Wₐ or W_{b} is an internucleotide linking group attaching the nucleotide represented by Formula IV to an oligonucleotide.

In certain embodiments of Formula IV, P₁, P₂, and P₃ are CH₂, and P₄ is O:

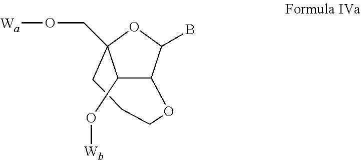

In certain embodiments of Formula IV, P₁ and P₃ are CH₂, P₂ is O and P₄ is O:

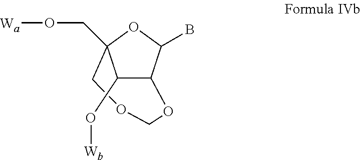

In certain embodiments, the bicyclic nucleotide has the structure of Formula Va or Vb:

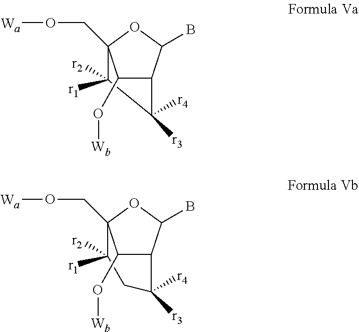

wherein B is a nucleobase;
wherein r1, r2, r3, and r4 are each independently H, halogen, C₁-C₁₂ alkyl, substituted C₁-C₁₂ alkyl, C₂-C₁₂ alkenyl, substituted C₂-C₁₂ alkenyl, C₂-C₁₂ alkynyl; substituted C₂-C₁₂ alkynyl; C₁-C₁₂ alkoxy; substituted C₁-C₁₂ alkoxy, OT₁, ST₁, SOT₁, SO₂T₁, NT₁T₂, N3, CN, C(═O)OT₁, C(═O)NT₁T₂, C(═O) T₁, O—C(═O)NT₁T2, N(H)C(═NH)NT₁T₂, N(H)C(═O)NT₁T₂ or N(H)C(═S)NT₁T₂, wherein each of T1 and T2 is independently H, C₁-C₆ alkyl, or substituted C₁-C₁₆ alkyl; or
r1 and r2 or r3 and r4 together are ═C(r5)(r6), wherein r5 and r6 are each independently H, halogen, C₁-C₁₂ alkyl, or substituted C₁-C₁₂ alkyl; and
wherein Wₐ and W_{b} are each independently, H, OH, a hydroxyl protecting group, a phosphorous moiety, or an internucleotide linking group attaching the nucleotide represented by Formula V to another nucleotide or to an oligonucleotide and wherein at least one of Wₐ or W_{b} is an internucleotide linking group attaching the nucleotide represented by Formula V to an oligonucleotide.

In certain embodiments, the bicyclic sugar moiety is a substituted furanosyl comprising a bridge that connects the 2′-carbon and the 4′-carbon of the furanosyl to form the second ring, wherein the bridge that connects the 2′-carbon and the 4′-carbon of the furanosyl includes, but is not limited to:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}), 4′-CH₂—N(CH₃)—O-2′ (also known as BNA^{NC}[NMe]), (as described in U.S. Pat. No. 7,427,672, which is hereby incorporated by reference in its entirety);
b) 4′-CH₂-2′; 4′-(CH₂)₂-2′; 4′-(CH₂)₃-2′; 4′-(CH₂)—O-2′ (also known as LNA); 4′-(CH₂)—S-2′; 4′-(CH₂)₂—O-2′ (also known as ENA); 4′-CH(CH₃)—O-2′ (also known as cEt); and 4′-CH(CH₂OCH₃)—O-2′ (also known as cMOE), and analogs thereof (as described in U.S. Pat. No. 7,399,845, which is hereby incorporated by reference in its entirety);
c) 4′-C(CH₃)(CH₃)—O-2′ and analogs thereof (as described in U.S. Pat. No. 8,278,283, which is hereby incorporated by reference in its entirety);
d) 4′-CH₂—N(OCH₃)-2′ and analogs thereof (as described in U.S. Pat. No. 8,278,425, which is hereby incorporated by reference in its entirety);
e) 4′-CH₂—O—N(CH₃)-2′ and analogs thereof (as described in U.S. Patent Publication No. 2004/0171570, which is hereby incorporated by reference in its entirety);
f) 4′-CH₂—C(H) (CH₃)-2′ and analogs thereof (as described in Chattopadhyaya et al., J. ORG. CHEM., 2009, 74, 118-34, which is hereby incorporated by reference in its entirety); and
g) 4′-CH₂—C(═CH₂)-2′ and analogs thereof as described in U.S. Pat. No. 8,278,426, which is hereby incorporated by reference in its entirety).

In certain embodiments, the bicyclic nucleotide (BN) is one or more of the following: (a) methyleneoxy BN, (b) ethyleneoxy BN, (c) aminooxy BN; (d) oxyamino BN, (e) methyl(methyleneoxy) BN (also known as constrained ethyl or cET), (f) methylene-thio BN, (g) methylene amino BN, (h) methyl carbocyclic BN, and (i) propylene carbocyclic BN, as shown below.

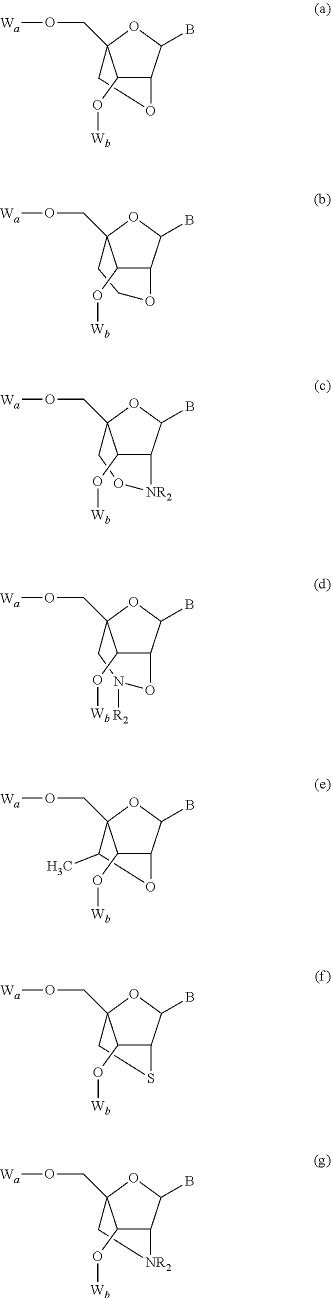

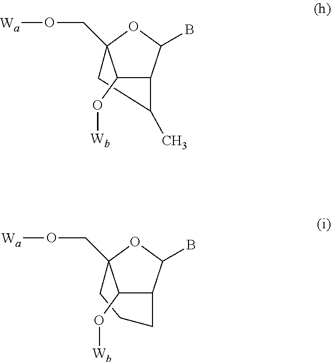

In the bicyclic nucleotides of (a) to (i) above, B is a nucleobase, R₂ is H or CH₃ and Wₐ and W_{b} are each independently, H, OH, a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

In one embodiment of the oxyamino BN (d), R₂ is CH₃, as follows (also known as BNA^{NC}[NMe]):

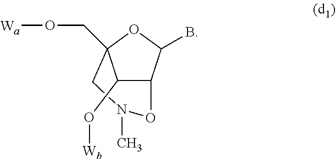

In certain embodiments, bicyclic sugar moieties and bicyclic nucleotides incorporating such bicyclic sugar moieties are further defined by isomeric configuration. In certain embodiments, the bicyclic sugar moiety or nucleotide is in the α-L configuration. In certain embodiments, the bicyclic sugar moiety or nucleotide is in the β-D configuration. For example, in certain embodiments, the bicyclic sugar moiety or nucleotide comprises a 2′O,4′-C-methylene bridge (2′-O—CH₂-4′) in the α-L configuration (α-L LNA). In certain embodiments, the bicyclic sugar moiety or nucleotide is in the R configuration. In certain embodiments, the bicyclic sugar moiety or nucleotide is in the S configuration. For example, in certain embodiments, the bicyclic sugar moiety or nucleotide comprises a 4′-CH(CH₃)—O-2′ bridge (i.e., cEt) in the S-configuration.

### Tricyclic Nucleotides

In some embodiments, the Tm-increasing nucleotide is a tricyclic nucleotide. The synthesis of tricyclic nucleotides and their incorporation into nucleic acid compounds has also been reported in the literature, including, for example, Steffens et al., J. AM. CHEM. SOC. 1997; 119:11548-549; Steffens et al., J. ORG. CHEM. 1999; 121 (14): 3249-55; Renneberg et al., J. AM. CHEM. SOC. 2002; 124:5993-6002; Ittig et al., NUCLEIC ACIDS RES. 2004; 32 (1): 346-53; Scheidegger et al., CHEMISTRY 2006; 12:8014-23; Ivanova et al., OLIGONUCLEOTIDES 2007; 17:54-65; each of which is each hereby incorporated by reference in its entirety.

In certain embodiments, the tricyclic nucleotide is a tricyclo nucleotide (also called tricyclo DNA) in which the 3′-carbon and 5′-carbon centers are connected by an ethylene that is fused to a cyclopropane ring, as discussed for example in Leumann C J, BIOORG. MED. CHEM. 2002; 10:841-54 and published U.S. Applications 2015/0259681 and 2018/0162897, which are each hereby incorporated by reference. In certain embodiments, the tricyclic nucleotide comprises a substituted furanosyl ring comprising a bridge that connects the 2′-carbon and the 4′-carbon of the furanosyl to form a second ring, and a third fused ring resulting from a group connecting the 5′-carbon to the methylene group of the bridge that connects the 2′-carbon and the 4′-carbon of the furanosyl, as discussed, for example, in published U.S. Application 2015/0112055, which is hereby incorporated by reference.

### Other Tₘ-Increasing Nucleotides

In addition to bicyclic and tricyclic nucleotides, other Tₘ-increasing nucleotides can be used in the RNAi oligonucleotides described herein. For example, in certain embodiments, the Tₘ-increasing nucleotide is a G-clamp, guanidine G-clamp or analogue thereof (Wilds et al., CHEM, 2002; 114:123 and Wilds et al., CHIM ACTA 2003; 114:123), a hexitol nucleotide (Herdewijn, CHEM. BIODIVERSITY 2010; 7:1-59), or a modified nucleotide. The modified nucleotide can have a modified nucleobase, as described herein, including for example, 5-bromo-uracil, 5-iodo-uracil, 5-propynyl-modified pyrimidines, or 2-amino adenine (also called 2,6-diaminopurine) (Deleavey et al., CHEM. & BIOL. 2012; 19:937-54) or 2-thio uridine, 5 Me-thio uridine, and pseudo uridine. The modified nucleotide can also have a modified sugar moiety, as described for example, in U.S. Pat. No. 8,975,389, which is hereby incorporated by reference, or as described herein, except that the Tₘ-increasing nucleotide is not modified at the 2′-carbon of the sugar moiety with a 2′-F or a 2′-OMe.

In certain embodiments, the Tₘ-increasing nucleotide is a bicyclic nucleotide. In certain embodiments, the Tₘ-increasing nucleotide is a tricyclic nucleotide. In certain embodiments, the Tₘ-increasing nucleotide a G-clamp, guanidine G-clamp or analogue thereof. In certain embodiments, the Tₘ-increasing nucleotide is a hexitol nucleotide. In certain embodiments, the Tₘ-increasing nucleotide is a bicyclic or tricyclic nucleotide. In certain embodiments, the Tₘ-increasing nucleotide is a bicyclic nucleotide, a tricyclic nucleotide, or a G-clamp, guanidine G-clamp or analogue thereof. In certain embodiments, the Tₘ-increasing nucleotide is a bicyclic nucleotide, a tricyclic nucleotide, a G-clamp, guanidine G-clamp or analogue thereof, or a hexitol nucleotide.

In certain embodiments, the Tₘ-increasing nucleotide increases the Tₘ of the nucleic acid inhibitor molecule by at least 2° C. per incorporation. In certain embodiments, the Tₘ-increasing nucleotide increases the Tₘ of nucleic acid inhibitor molecule by at least 3° C. per incorporation. In certain embodiments, the Tₘ-increasing nucleotide increases the Tₘ of nucleic acid inhibitor molecule by at least 4° C. per incorporation. In certain embodiments, the Tₘ-increasing nucleotide increases the Tₘ of nucleic acid inhibitor molecule by at least 5° C. per incorporation.

### Targeting Ligands

In some embodiments, it is desirable to target the oligonucleotides of the disclosure (e.g., lipid-conjugated RNAi oligonucleotides) to one or more cells or tissues of the central nervous system (CNS). In some embodiments, it is desirable to target the oligonucleotides of the disclosure (e.g., lipid-conjugated RNAi oligonucleotides) to one or more cells or tissues of the liver. In some embodiments, it is desirable to target the oligonucleotides of the disclosure (e.g., lipid-conjugated RNAi oligonucleotides) to one or more cells or tissues of the eye.

Such a strategy can help to avoid undesirable effects in other organs or avoid undue loss of the oligonucleotide to cells, tissue or organs that would not benefit from the oligonucleotide. Accordingly, in some embodiments, a lipid-conjugated RNAi oligonucleotide disclosed herein is modified to facilitate targeting and/or delivery to a particular tissue, cell, or organ (e.g., to facilitate delivery of the conjugate to the CNS). In some embodiments, a lipid-conjugated RNAi oligonucleotide comprises at least one nucleotide (e.g., 1, 2, 3, 4, 5, 6 or more nucleotides) conjugated to one or more targeting ligand(s).

In some embodiments, 1 or more (e.g., 1, 2, 3, 4, 5 or 6) nucleotides of a lipid-conjugated RNAi oligonucleotide disclosed herein are each conjugated to a separate targeting ligand. In some embodiments, 1 nucleotide of a lipid-conjugated RNAi oligonucleotide herein is conjugated to a separate targeting ligand. In some embodiments, 2 to 4 nucleotides of a lipid-conjugated RNAi oligonucleotide herein are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the lipid-conjugated RNAi oligonucleotide resembles a toothbrush. For example, a lipid-conjugated RNAi oligonucleotide may comprise a stem-loop at either the 5′ or 3′ end of the sense strand and 1, 2, 3 or 4 nucleotides of the loop of the stem may be individually conjugated to a targeting ligand. In some embodiments, a lipid-conjugated RNAi oligonucleotide provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotide of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides. In some embodiments, the tetraloop is any combination of adenine, guanine, cytosine, and uridine nucleotides.

In some embodiments, the tetraloop (L) has a monovalent GalNAc moiety attached to any one or more guanine nucleotides of the tetraloop via any linker described herein, as depicted below (X=heteroatom):

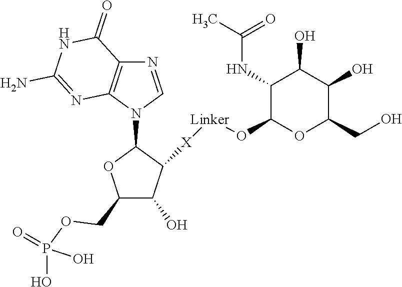

In some embodiments, the tetraloop (L) has a monovalent GalNAc attached to any one or more adenine nucleotides of the tetraloop via any linker described herein, as depicted below (X=heteroatom):

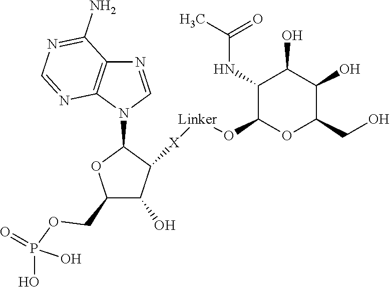

In some embodiments, a lipid-conjugated RNAi oligonucleotide herein comprises a monovalent GalNAc attached to a guanine nucleotide referred to as [ademG-GalNAc] or 2′-aminodiethoxymethanol-Guanine-GalNAc, as depicted below:

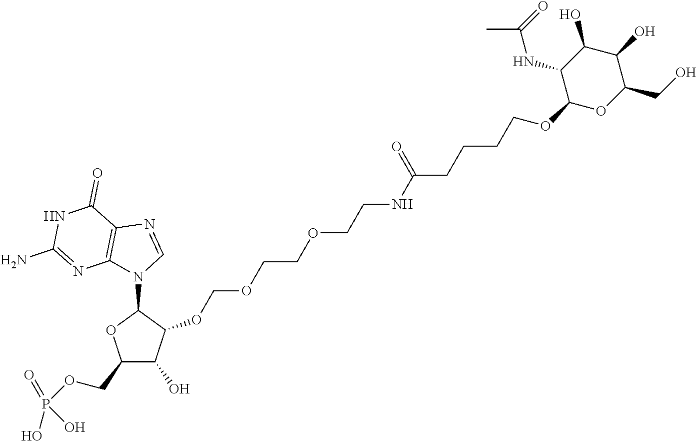 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, a lipid-conjugated RNAi oligonucleotide herein comprises a monovalent GalNAc attached to an adenine nucleotide, referred to as [ademA-GalNAc] or 2′-aminodiethoxymethanol-Adenine-GalNAc, as depicted below:

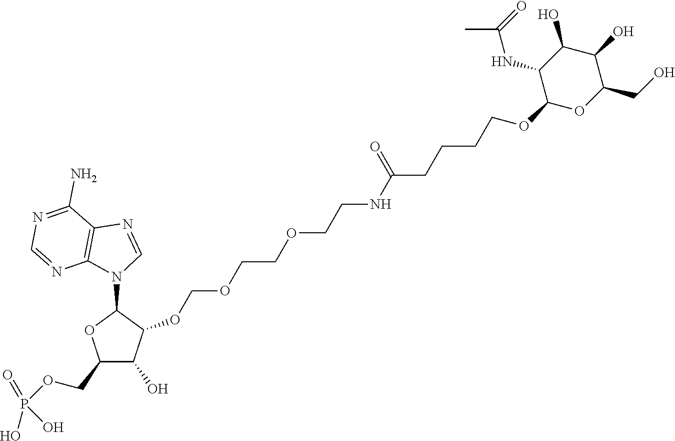 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

An example of such conjugation is shown below for a loop comprising from 5′ to 3′ the nucleotide sequence GAAA (L=linker, X=heteroatom). Such a loop may be present, for is used to describe example, at positions 27-30 of the sense strand. In the chemical formula,

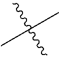

an attachment point to the oligonucleotide strand.

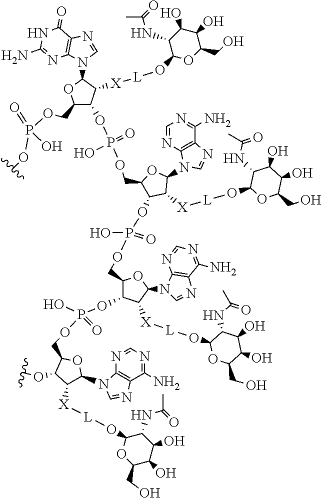

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to nucleotides of the loop using an acetal linker. Such a loop may be present, for example, at positions 27-30 of the sense strand. In the chemical formula,

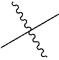

is an attachment point to the oligonucleotide strand.

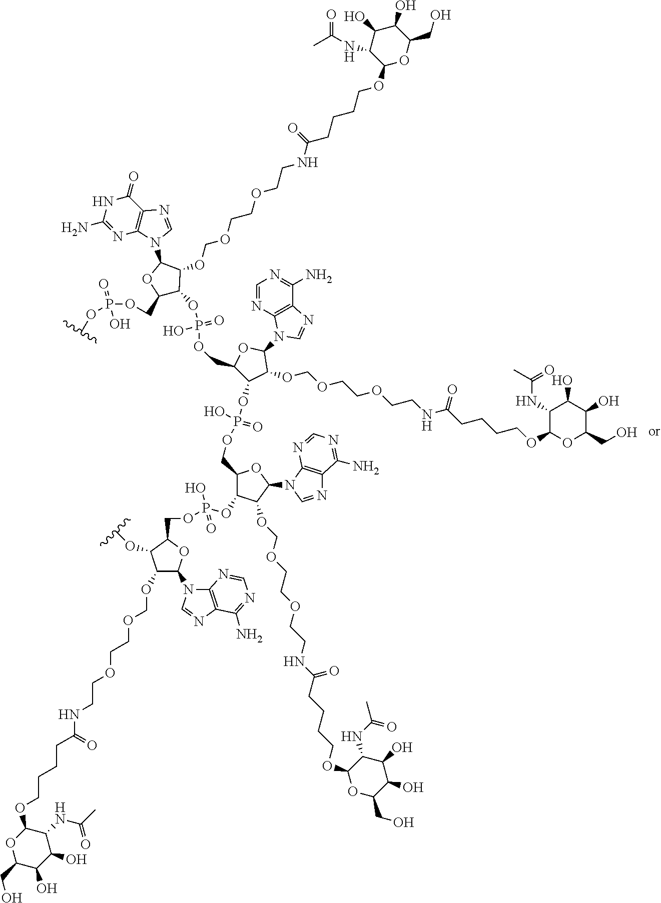

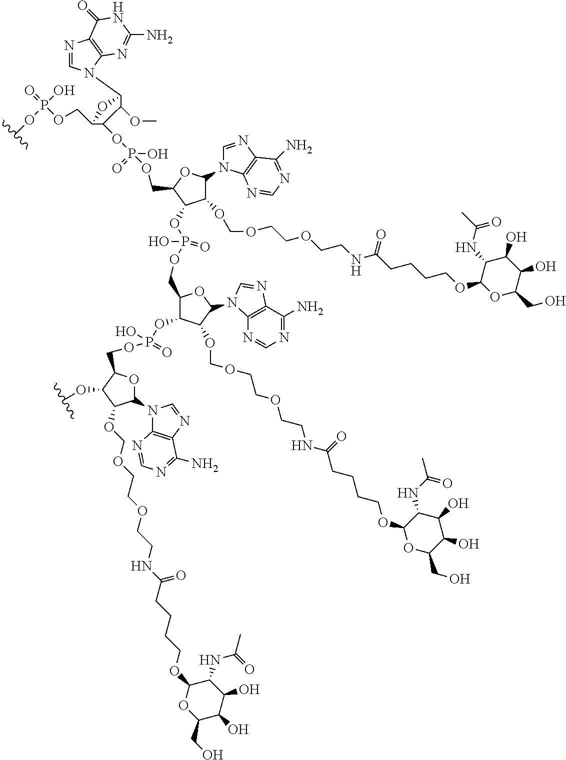

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a lipid-conjugated RNAi oligonucleotide. In some embodiments, a lipid-conjugated RNAi oligonucleotide herein does not have a GalNAc conjugated thereto.

### Lipid Conjugates

In some embodiments, any of the lipid moieties described herein are conjugated to a nucleotide of the sense strand of the oligonucleotide. In some embodiments, a lipid moiety is conjugated to a terminal position of the oligonucleotide. In some embodiments, the lipid moiety is conjugated to the 5′ terminal nucleotide of the sense strand. In some embodiments, the lipid moiety is conjugated to the 3′ terminal nucleotide of the sense strand.

In some embodiments, the lipid moiety is conjugated to an internal nucleotide on the sense strand. An internal position is any nucleotide position other than the two terminal positions from each end of the sense strand. In some embodiments, the lipid moiety is conjugated to one or more internal positions of the sense strand. In some embodiments, the lipid moiety is conjugated to position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37 or position 38 of a sense strand. In some embodiments, the lipid moiety is conjugated to position 1 of the sense strand. In some embodiments, the lipid moiety is conjugated to position 2 of the sense strand. In some embodiments, the lipid moiety is conjugated to position 4 of the sense strand. In some embodiments, the lipid moiety is conjugated to position 6 of the sense strand. In some embodiments, the lipid moiety is conjugated to position 8 of the sense strand. In some embodiments, the lipid moiety is conjugated to position 15 of the sense strand. In some embodiments, the lipid moiety is conjugated to position 28 of the sense strand. In some embodiments, the lipid moiety is conjugated to position 38 of the sense strand.

In some embodiments, the lipid moiety is conjugated to a nucleotide of the sense strand that forms a base pair with a nucleotide at position 20, position 19, position 18, position 17, position 16, position 15, position 14, position 13, or position 12 of the antisense strand. In some embodiments, the lipid moiety is conjugated to a nucleotide of the sense strand that forms a base pair with a nucleotide at position 16, position 14, or position 12 of the antisense strand. In some embodiments, the lipid moiety is conjugated to a nucleotide of the sense strand that forms a base pair with a nucleotide at position 20 of the antisense strand. In some embodiments, the lipid moiety is conjugated to a nucleotide of the sense strand that forms a base pair with a nucleotide at position 19 of the antisense strand. In some embodiments, the lipid moiety is conjugated to a nucleotide of the sense strand that forms a base pair with a nucleotide at position 18 of the antisense strand. In some embodiments, the lipid moiety is conjugated to a nucleotide of the sense strand that forms a base pair with a nucleotide at position 17 of the antisense strand. In some embodiments, the lipid moiety is conjugated to a nucleotide of the sense strand that forms a base pair with a nucleotide at position 16 of the antisense strand. In some embodiments, the lipid moiety is conjugated to a nucleotide of the sense strand that forms a base pair with a nucleotide at position 15 of the antisense strand. In some embodiments, the lipid moiety is conjugated to a nucleotide of the sense strand that forms a base pair with a nucleotide at position 14 of the antisense strand. In some embodiments, the lipid moiety is conjugated to a nucleotide of the sense strand that forms a base pair with a nucleotide at position 13 of the antisense strand. In some embodiments, the lipid moiety is conjugated to a nucleotide of the sense strand that forms a base pair with a nucleotide at position 12 of the antisense strand.

In some embodiments, a lipid-conjugated RNAi oligonucleotide described herein comprises at least one nucleotide conjugated with one or more lipid moieties. In some embodiments, the one or more lipid moieties are conjugated to the same nucleotide. In some embodiments, the one or more lipid moieties are conjugated to different nucleotides. In some embodiments, one, two, three, four, five, or six lipid moieties are conjugated to the oligonucleotide. In some embodiments, one or more lipid moieties are conjugated to an adenine nucleotide. In some embodiments, one or more lipid moieties are conjugated to a guanine nucleotide. In some embodiments, one or more lipid moieties are conjugated to a cytosine nucleotide. In some embodiments, one or more lipid moieties are conjugated to a thymine nucleotide. In some embodiments, one or more lipid moieties are conjugated to a uracil nucleotide.

In some embodiments, the lipid moiety is a hydrocarbon chain. In some embodiments, the hydrocarbon chain is saturated. In some embodiments, the hydrocarbon chain is unsaturated. In some embodiments, the hydrocarbon chain is branched. In some embodiments, the hydrocarbon chain is straight. In some embodiments, the lipid moiety is a C8-C30 hydrocarbon chain. In some embodiments, the lipid moiety is a C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1.

In some embodiments, the lipid moiety is a C16 hydrocarbon chain.

In some embodiments, the lipid moiety is conjugated to the oligonucleotide via a linker. In some embodiments, a nucleotide of the lipid-conjugated oligonucleotide is represented by formula II-b or II-c:

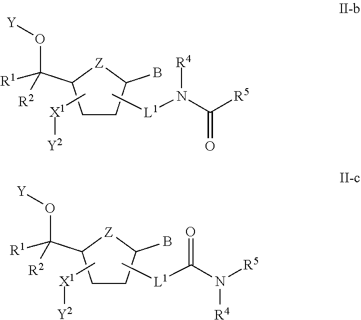

or a pharmaceutically acceptable salt thereof, wherein:
L1 is a covalent bond, a monovalent or a bivalent saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

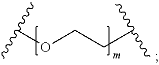

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments of the lipid-conjugated RNAi oligonucleotide. R⁵ is selected from

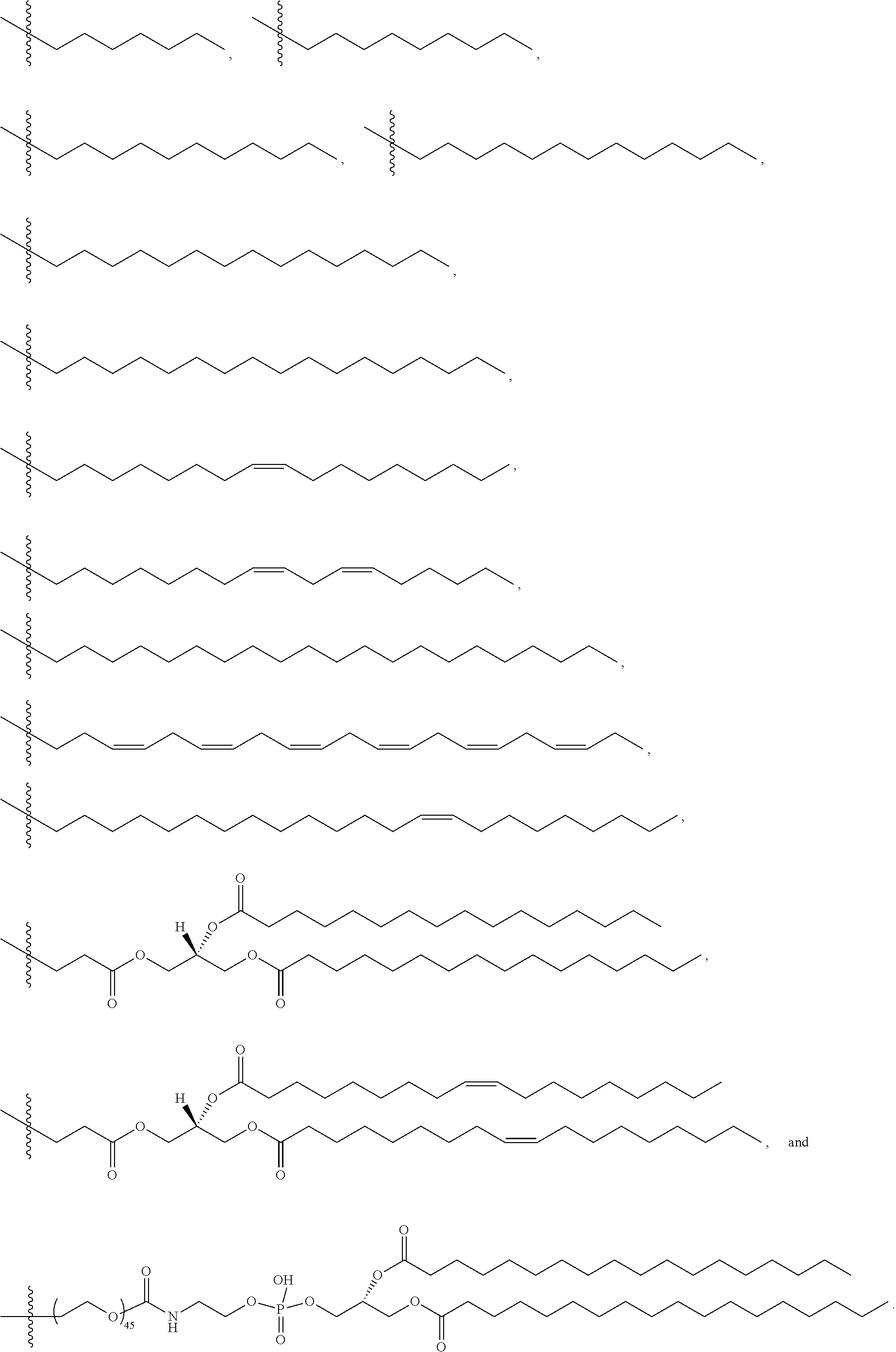

In certain embodiments of the lipid-conjugated RNAi oligonucleotide.
R⁵ is selected from

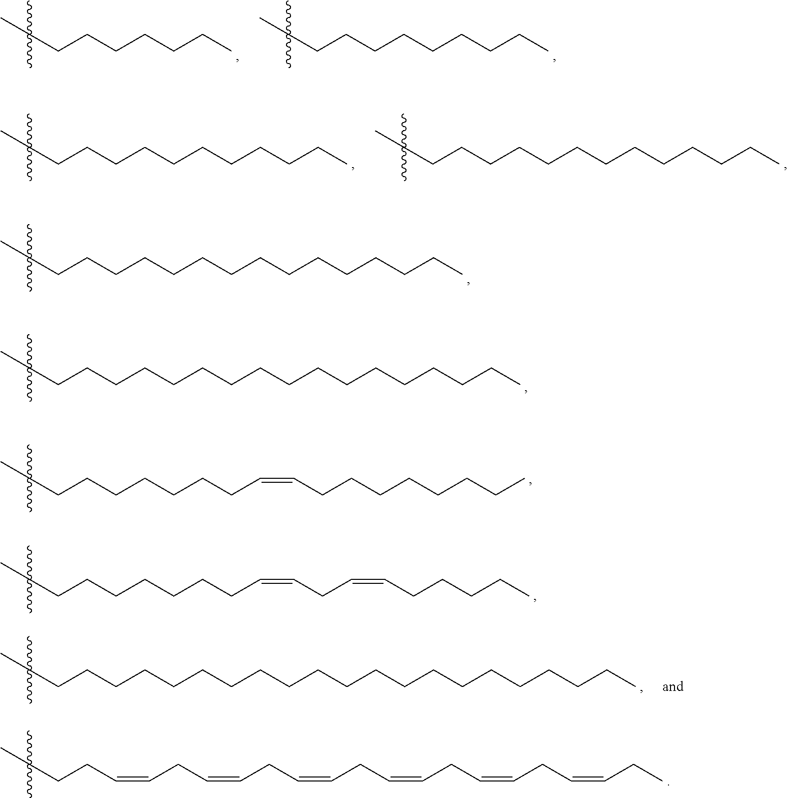

In some embodiments. R⁵ is

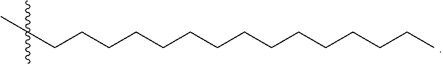 [SMILES: *CCCCCCCCCCCCCCC]

In some embodiments. R⁵ is

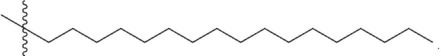 [SMILES: *CCCCCCCCCCCCCCCCC]

In some embodiments. R⁵ is.

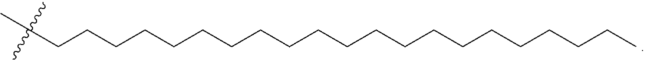 [SMILES: *CCCCCCCCCCCCCCCCCCCCC]

In some embodiments, a nucleotide of the lipid-conjugated RNAi oligonucleotide is represented by formula II-Ib or II-Ic:

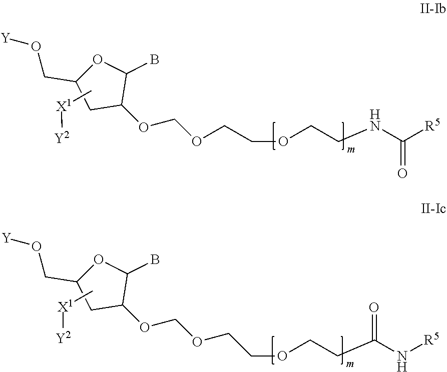

or a pharmaceutically acceptable salt thereof; wherein
B is a nucleobase or hydrogen;
m is 1-50;
X¹ is —O—, or —S—;
Y is hydrogen,

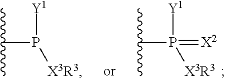

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, the lipid is

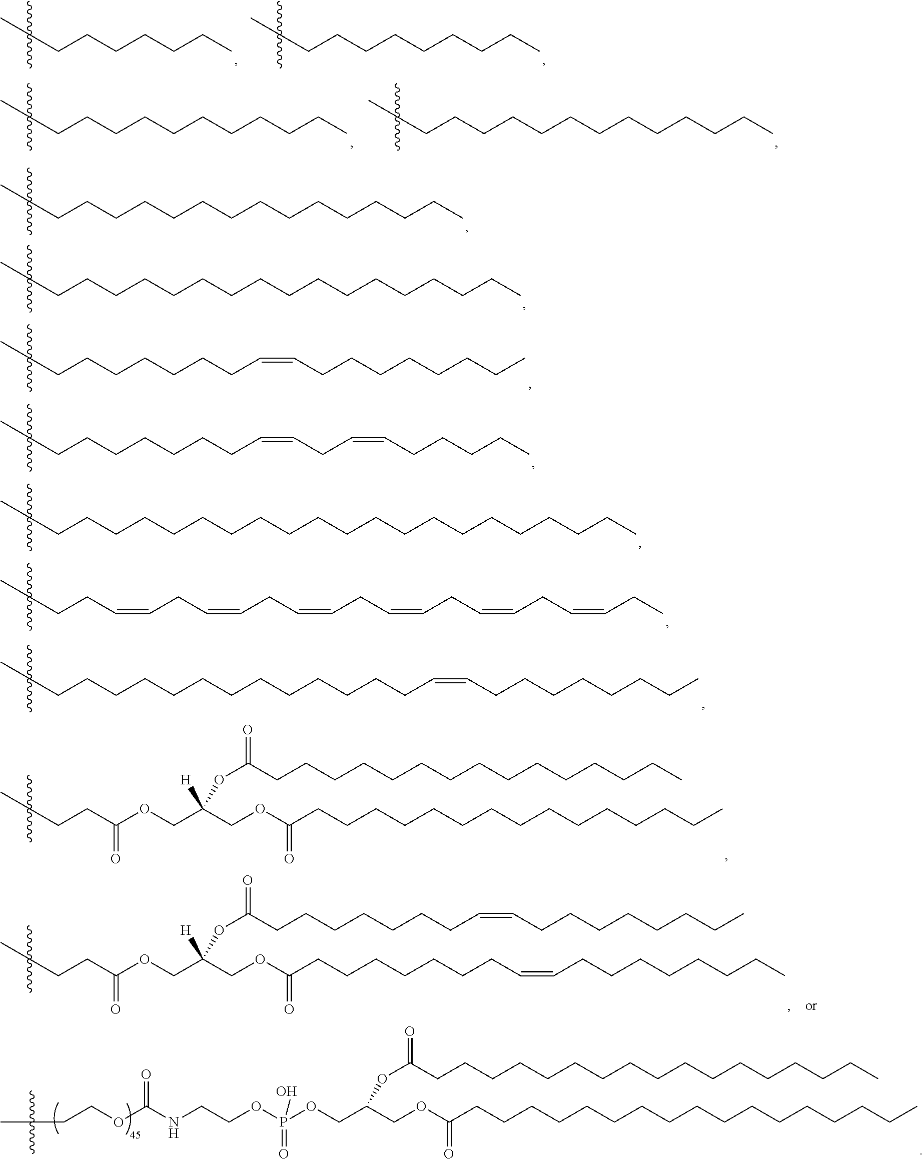

In some embodiments, the oligonucleotide of the oligonucleotide-ligand conjugate is a double-stranded molecule. In some embodiments, the oligonucleotide is an RNAi molecule. In some embodiments, the double stranded oligonucleotide comprises a stem loop. In some embodiments, the stem loop is set forth as S1-L-S2, wherein S1 is complementary to S2, and wherein L forms a loop between S1 and S2. In some embodiments, the ligand is conjugated to any of the nucleotides in the loop of the stem loop. In some embodiments, the ligand is conjugated to any of the nucleotides in the stem of the stem loop. In some embodiments, the ligand is conjugated to the first nucleotide from 5′ to 3′ in the loop. In some embodiments, the ligand is conjugated to the second nucleotide from 5′ to 3′ in the loop. In some embodiments, the ligand is conjugated to the third nucleotide from 5′ to 3′ in the loop. In some embodiments, the ligand is conjugated to the fourth nucleotide from 5′ to 3′ in the loop. In some embodiments, the ligand is conjugated to one, two, three, or four of the nucleotides in the loop. In some embodiments, the ligand is conjugated to three of the nucleotides in the stem loop.

In some embodiments, the stem loop is 16 nucleotides in length. In some embodiments, the ligand is conjugated to the third nucleotide from 5′ to 3′ in the stem loop. In some embodiments, the ligand is conjugated to the eighth nucleotide from 5′ to 3′ in the stem loop. In some embodiments, the ligand is conjugated to the ninth nucleotide from 5′ to 3′ in the stem loop. In some embodiments, the ligand is conjugated to the tenth nucleotide from 5′ to 3′ in the stem loop.

### Exemplary Oligonucleotides

In some embodiments, the lipid-conjugated RNAi oligonucleotide comprises a nucleotide conjugated with a fatty acid. In some embodiments, the fatty acid is a saturated fatty acid. In some embodiments, the fatty acid is an unsaturated fatty acid. In some embodiments, lipid-conjugated RNAi oligonucleotide comprises a nucleotide conjugated with a lipid. In some embodiments, the lipid is a carbon chain. In some embodiments, the carbon chain is saturated. In some embodiments, the carbon chain is unsaturated. In some embodiments, the lipid-conjugated RNAi oligonucleotide comprises a nucleotide conjugated with a 16-carbon (C16) lipid. In some embodiments, the C16 lipid comprises at least one double bond. In some embodiments, the lipid-conjugated RNAi oligonucleotide comprises a nucleotide conjugated with a 22-carbon (C22) lipid.

In some embodiments, the oligonucleotide of the lipid-conjugated RNAi oligonucleotide is conjugated to a C16 lipid as shown in:

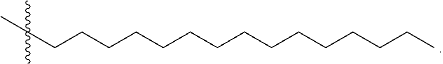 [SMILES: *CCCCCCCCCCCCCCC]

In some embodiments, the oligonucleotide of the lipid-conjugated RNAi oligonucleotide is conjugated to a C22 lipid as shown in:

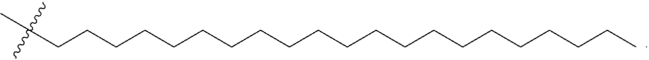 [SMILES: *CCCCCCCCCCCCCCCCCCCCC]

In some embodiments, the 3′ end of the sense strand is a blunt end. In some embodiments, the 5′ end of the antisense strand is a blunt end. In some embodiments, the 3′ end of the antisense strand comprises an overhang. In some embodiments, the 5′ end of the antisense strand comprises an overhang. In some embodiments, the 5′ and 3′ ends of the antisense strand each comprise an overhang.

In some embodiments, the lipid-conjugated RNAi oligonucleotide comprises one or more 2′ modifications. In some embodiments, the 2′ modifications are selected from 2′-fluoro and 2′-methyl.

In some embodiments, a lipid-conjugated RNAi oligonucleotide for reducing expression of a target gene comprises the modification pattern of

Sense Strand:
5′-[mXs][mXs][mX][mX][mX][ademX-L][mX][mX][mX][mX]
[mX][mX][mX][mX][mX][mX][mX][mX][mX][mX][mX][fX]
[fX][fX][fX][mX][mX][mX][mX][mX][mX][mXs][mXs]
[mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-L]=Lipid attached to a nucleotide.

In some embodiments, a lipid-conjugated RNAi oligonucleotide for reducing expression of a target gene comprises the modification pattern of

Sense Strand:
5′-[+X][mX][mX][ademX-L][mX][mX][mX][+X][mX][mX]
[mX][mX][mX][mX][mX][mX][mX][fX][fX][fX][fX][mX]
[mX][mX][mX][mXs][mXs][mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5′-[+X][mX][mX][mX][mX][ademX-L][mX][mX][mX][mX]
[mX][+X][mX][mX][mX][mX][mX][mX][mX][mX][mX][fX]
[fX][fX][fX][mX][mX][mX][mX][mX][mX][mXs][mXs]
[mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5′-[ademX-Ls][mX][mX][mX][mX][mX][mX][fX][fX][fX]
[fX][mX][mX][mX][mX][mXs][mXs][mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-Ls][mX][mX][mX][mX][mX][mX][fX][fX][fX]
[fX][mX][mX][mX][mXs][mXs][mX]-3′

### Hybridized to:

Antisense Strand:
5'-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-Ls][+X][mX][mX][mX][mX][mX][fX][fX][fX]
[fX][mX][mX][mXs][+Xs][+X]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-Ls]=Lipid attached to a nucleotide with a phosphorothioate linkage to the neighboring nucleotide, [+X]=a Tm-increasing nucleotide, optionally an LNA, and [+Xs]=a Tm-increasing nucleotide with a phosphorothioate linkage to the neighboring nucleotide, optionally an LNA or BNA.

In some embodiments, a lipid-conjugated RNAi oligonucleotide for reducing expression of a target gene comprises the modification pattern of

Sense Strand:
5′-[ademX-Ls][mX][mX][mX][mX][mX][mX][fX][fX][fX]
[fX][mX][mX][mX][mX][mXs][mXs][mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-Ls][mX][mX][mX][mX][mX][mX][fX][fX][fX]
[fX][mX][mX][mX][mX][mX][mX][mX][mX][mX][mX][mX]
[mX][mX][mX][mX][mX][mX][mX][mX][mX][mX][mX][mX]
[mX][mX]-3′

### Hybridized to:

Antisense Strand:
5'-[MePhosphonate-4O-mXs][fXs][fXs][fX][X][mX]
[fX][mX][mX][fX][mX][mX][mX][fX][mX][mX][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5'-[+Xs][ademX-L][fX][X][fX][fX][mX][mX][mX][X]
[+Xs][mXs][mX]-3'

### Hybridized to:

Antisense Strand:
5'-[MePhosphonate-4O-mXs][fXs][fXs][fX][fX][mX]
[fX][mX][mX][fX][mX][X][mX][fX][mX][X][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5'-[+Xs][ademX-L][fX][X][fX][fX][mX][mX][mX][+X]
[+Xs][mXs][mX]-3'

### Hybridized to:

Antisense Strand:
5'-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-L]=Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5'-[ademX-Ls][mX][mX][mX][mX][mX][mX][X][fX][fX]
[fX][mX][mX][mX][mX][mX][mX][mXs][mXs][mX]-3'

### Hybridized to:

Antisense Strand:
5'-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:5'-[ademX-Ls][mX][mX][mX][mX][mX]
[mX][fX][fX][fX][fX][mX][mX][X][mX][mXs][mXs]
[mX]-3'

### Hybridized to:

Antisense Strand:
5'-[MePhosphonate-4O-mXs][fXs][fXs][fX][fX][mX]
[fX][X][mX][fX][mX][mX][mX][fX][mX][mX][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-Ls]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5'-[mXs][mXs][mX][mX][mX][ademX-C16][mX][mX][mX]
[mX][mX][mX][mX][mX][X][X][mX][mX][mX][mX][mX]
[fX][X][fX][fX][mX][mX][mX][mX][mX][X][mXs][mXs]
[mX]-3'

### Hybridized to:

Antisense Strand:
5'-[MePhosphonate-4O-mXs][fXs][fXs][fX][fX][mX]
[fX][mX][mX][fX][mX][X][mX][X][mX][mX][X][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-C16]=C16 Lipid attached to a nucleotide.

In some embodiments, a lipid-conjugated RNAi oligonucleotide for reducing expression of a target gene comprises the modification pattern of

Sense Strand:
5'-[+X][mX][mX][ademX-C16][mX][X][X][+X][mX][mX]
[mX][X][mX][mX][mX][mX][mX][fX][fX][fX][fX][mX]
[mX][mX][mX][mXs][mXs][mX]-3'

### Hybridized to:

Antisense Strand:
5'-[MePhosphonate-4O-mXs][fXs][fXs][fX][fX][mX]
[fX][mX][mX][fX][mX][mX][mX][fX][mX][mX][mX]
[mX][mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-C16]=C16 Lipid attached to a nucleotide, [+X]=a Tm-increasing nucleotide, optionally an LNA.

In some embodiments, a lipid-conjugated RNAi oligonucleotide for reducing expression of a target gene comprises the modification pattern of

Sense Strand:
5'-[+X][mX][X][mX][mX][ademX-C16][mX][mX][mX]
[mX][mX][+X][mX][mX][mX][X][mX][mX][mX][mX][mX]
[fX][X][fX][fX][mX][mX][mX][mX][mX][mX][mXs][mXs]
[mX]-3'

### Hybridized to:

Antisense Strand:
5'-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3'

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C16]=C16 Lipid attached to a nucleotide, [+X]=a Tm-increasing nucleotide, optionally an LNA.

In some embodiments, a lipid-conjugated RNAi oligonucleotide for reducing expression of a target gene comprises the modification pattern of

Sense Strand:
5′-[ademX-C16s][mX][mX][mX][mX][mX][mX][fX][fX]
[fX][fX][mX][mX][mX][mX][mXs][mXs][mX]-3′

### Hybridized to:

Antisense Strand:
5′-[MePhosphonate-4O-mXs][fXs][fXs][fX][fX]
[mX][fX][mX][mX][fX][mX][mX][mX][fX][mX][mX]
[mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C16s]=C16 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-C16s][mX][mX][mX][mX][mX][mX][fX][fX]
[fX][fX][mX][mX][mX][mXs][mXs][mX]-3′

### Hybridized to:

Antisense Strand:
5′-[MePhosphonate-4O-
mXs][fXs][fXs][fX][fX][mX][mX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C16s]=C16 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-C16s][+X][mX][mX][mX][mX][mX][fX][fX]
[fX][fX][mX][mX][mXs][+Xs][+X]-3′

### Hybridized to:

Antisense Strand:
5′-[MePhosphonate-4O-
mXs][fXs][fXs][fX][fX][mX][fX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C16s]=C16 lipid attached to a nucleotide with a phosphorothioate linkage to the neighboring nucleotide, [+X]=a Tm-increasing nucleotide, optionally an LNA, and [+Xs]=a Tm-increasing nucleotide with a phosphorothioate linkage to the neighboring nucleotide, optionally an LNA or BNA.

In some embodiments, a lipid-conjugated RNAi oligonucleotide for reducing expression of a target gene comprises the modification pattern of

Sense Strand:
5′-[ademX-C22s][mX][mX][mX][mX][mX][fX][fX][fX]
[fX][mX][mX][mX][mX][mX][mXs][mXs][mX]-3′

### Hybridized to:

Antisense Strand:
5′-[MePhosphonate-4O-
mXs][fXs][fXs][fX][fX][mX][fX][mX][mX][fX][mX]
[mX][mX][fX][mX][mX][mX][mX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22s]=C22 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[ademX-C22s][mX][mX][mX][mX][mX][mX][fX][fX]
[fX][fX][mX][mX][mX][mX][mX][mX][mX][mX][mX][mX]
[mX][mX][mX][mX][mX][mX][mX][mX][mX][mX][mX][mX]
[mX][mX][mX]-3′

### Hybridized to:

Antisense Strand:
5′-[MePhosphonate-4O-mXs][fXs][fXs][fX][fX][mX]
[mX][mX][mX][mX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22s]=C22 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[+Xs][ademX-C22][fX]
[fX][fX][fX][mX][mX][mX][mX][+Xs][mXs][mX]-3′

### Hybridized to:

Antisense Strand:
5′-[MePhosphonate-4O-mXs][fXs][fXs][fX][fX][mX]
[mX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22]=C22 Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5′-[+Xs][ademX-C22][fX]
[fX][fX][fX][mX][mX][mX][+X][+Xs][mXs][mX]-3′

### Hybridized to:

Antisense Strand:
5′-[MePhosphonate-4O0-mXs][fXs][fXs][fX][fX][mX]
[fX][mX][mX][fX][mX][mX][mX][fX][mX][mX][mX]
[MX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22]=C22 Lipid attached to a nucleotide, [+X]=a Tm-increasing nucleotide, optionally an LNA, and [+Xs]=a Tm-increasing nucleotide with a phosphorothioate linkage to the neighboring nucleotide, optionally an LNA.

In some embodiments, a lipid-conjugated RNAi oligonucleotide for reducing expression of a target gene comprises the modification pattern of

Sense Strand:
5′-[ademX-C22s][mX][mX][mX][mX][mX][mX][fX][fX]
[fX][fX][mX][mX][mX][mX][mX][mX][mXs][mXs][mX]-3′

### Hybridized to:

Antisense Strand:
5′ - [MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22s]=C22 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′- [ademX-C22s][mX][mX][mX][mX][mX][mX][X][fX]
[fX][fX][mX][mX][mX][mX][mXs][mXs][mX]- 3′

### Hybridized to:

Antisense Strand:
5′ - [MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-C22s]=C22 Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′-[mX][mX][mX][X][fX][fX][fX][mX][mX][mX][mX][mX]
[mX][mX][mX][X][mX][mX][mX][mX][mX][mX][mX][ademX-
L][mX][mX][mX][mX][mX][mX][mX][mX]-3′

### Hybridized to:

Antisense Strand:
5′ - [MePhosphonate-4O-mXs][fXs][fXs][fX][fX][mX]
[fX][X][mX][fX][mX][mX][mX][fX][mX][mX/mXs][mX/
mXs][mX/mXs][mX/mXs][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/mXs]=the position is either a 2′-O-methyl modified nucleotide with phosphodiester linkages to neighboring nucleotides or a 2′-O-methyl modified nucleotide with phosphorothioate linkages to neighboring nucleotides, and [ademX-L]=Lipid attached to a nucleotide.

In some embodiments, a lipid-conjugated RNAi oligonucleotide for reducing expression of a target gene comprises the modification pattern of

Sense Strand:
5′-[+Xs][fX][fX][fX][fX][X][mX][X][X][X][mX][mX]
[mX][mX][mX][mX][mX][mX][mX][mX][mX][ademX-L][mX]
[mX][mX][X][mX][mX][mX][mX]-3′

### Hybridized to:

Antisense Strand:
5′ - [MePhosphonate-4O-mXs][fXs][fXs][fX][fX][mX]
[fX][mX][mX][fX][mX][mX][mXs][fXs][mX/mXs][mX/mXs]
[mX/mXs][mX/mXs][mX/mXs][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/mXs]=the position is either a 2′-O-methyl modified nucleotide with phosphodiester linkages to neighboring nucleotides or a 2′-O-methyl modified nucleotide with phosphorothioate linkages to neighboring nucleotides, and [ademX-L]=Lipid attached to a nucleotide.

In some embodiments, a lipid-conjugated RNAi oligonucleotide for reducing expression of a target gene comprises the modification pattern of

Sense Strand:
5′-[+Xs][fX][fX][X][mX][mX][mX][mX][mX][mX][mX]
[mX][mX][mX][mX][mX][mX][mX][mX][ademX-L][mX][mX]
[mX][mX][mX][mX][mX][mX]-3′

### Hybridized to:

Antisense Strand:
5′ - [MePhosphonate-4O-mXs][fXs][fXs][fX][fX][mX]
[X][mX][mX][fX][X][mX/mXs][mXs][fXs][mX/mXs][mX/
mXs][mX/mXs][mX/mXs][mX/mXs][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/mXs]=the position is either a 2′-O-methyl modified nucleotide with phosphodiester linkages to neighboring nucleotides or a 2′-O-methyl modified nucleotide with phosphorothioate linkages to neighboring nucleotides, and [ademX-L]=Lipid attached to a nucleotide.

In some embodiments, a lipid-conjugated RNAi oligonucleotide for reducing expression of a target gene comprises the modification pattern of

Sense Strand:
5′- [ademXs-L][mX/+X][mX][mX][X][mX][mX][fX][fX]
[fX][fX][mX][mX][mX][mX/+X][mX/+X][mX][mXs/+Xs]
[mXs/+Xs][mX]-3′

### Hybridized to:

Antisense Strand:
5′ - [MePhosphonate-4O-mXs][fXs][fXs][fX][fX][mX]
[fX][mX][mX][fX][mX][X][X][fX][mX][mX][mX][mX][mX]
[mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/+X]=the position is either a 2′-O-methyl modified nucleotide with phosphodiester linkages to neighboring nucleotides or a Tₘ-increasing nucleotide with phosphodiester linkages to neighboring nucleotides, optionally an LNA, and [ademXs-C16]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′- [ademXs-L][fX/+X][fX][fX][fX][mX][mX][mX][mX/
+X][mX/+X][X][mXs/+Xs][mXs/+Xs][mX]-3′

### Hybridized to:

Antisense Strand:
5′ - [MePhosphonate-4O-mXs][fXs][fXs][fX][fX][mX]
[fX][mX][mX][fX][mX][mX][mXs][fXs][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/+X]=the position is either a 2′-O-methyl modified nucleotide with phosphodiester linkages to neighboring nucleotides or a Tm-increasing nucleotide, [mXs/+Xs]=the position is either a 2′-O-methyl modified nucleotide with a phosphorothioate linkage to the neighboring nucleotide or a Tm-increasing nucleotide with phosphorothioate linkage to neighboring nucleotide, optionally an LNA, [fXs/+Xs]=the position is either a 2′-fluoro modified nucleotide with a phosphorothioate linkage to the neighboring nucleotide or a Tm-increasing nucleotide with phosphorothioate linkage to neighboring nucleotide, optionally an LNA and [ademXs-L]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′- [ademXs-L][mX/+X][mX][fX][fX][fX][fX][mX][mX]
[mX][mX/+X][mX/+X][X][mXs/+Xs][mXs/+Xs][mX]-3′

### Hybridized to:

Antisense Strand:
5′ - [MePhosphonate-4O-mXs][fXs][fXs][fX][fX][mX]
[fX][mX][mX][fX][mX][mX][X][fX][mX][X][mX][X][mX]
[mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/+X]=the position is either a 2′-O-methyl modified nucleotide with phosphodiester linkages to neighboring nucleotides or a Tm-increasing nucleotide, [mXs/+Xs]=the position is either a 2′-O-methyl modified nucleotide with a phosphorothioate linkage to the neighboring nucleotide or a Tm-increasing nucleotide with phosphorothioate linkage to neighboring nucleotide, optionally an LNA, and [ademXs-L]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′- [ademXs-L][mX/+X][mX][fX][mX][mX][mX][mX/+X]
[mX/+X][mX][mXs/+Xs][mXs/+Xs][mX]-3′

### Hybridized to:

Antisense Strand:
5′ - [MePhosphonate-4O-mXs][fXs][fXs][fX][fX][X]
[fX][mX][mX][fX][X][mX][mXs][fXs][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [mX/+X]=the position is either a 2′-O-methyl modified nucleotide with phosphodiester linkages to neighboring nucleotides or a Tm-increasing nucleotide, [mXs/+Xs]=the position is either a 2′-O-methyl modified nucleotide with a phosphorothioate linkage to the neighboring nucleotide or a Tm-increasing nucleotide with phosphorothioate linkage to neighboring nucleotide, optionally an LNA, [fXs/+Xs]=the position is either a 2′-fluoro modified nucleotide with a phosphorothioate linkage to the neighboring nucleotide or a Tm-increasing nucleotide with phosphorothioate linkage to neighboring nucleotide, optionally an LNA and [ademXs-L]=Lipid attached to a nucleotide with a phosphorothioate linkage to the neighboring nucleotide.

In some embodiments, a lipid-conjugated RNAi oligonucleotide for reducing expression of a target gene comprises the modification pattern of

Sense Strand:
5′- [+Xs][ademX-L][fX][fX][fX][fX][mX][mX][mX][mX]
[+Xs][mXs][mX]-3′

### Hybridized to:

Antisense Strand:
5′ - [MePhosphonate-4O-mXs][fXs][fXs][fX][fX][mX]
[fX][mX][mX][fX][mX][mX][X][fX][mX][X][mX][mX][mX]
[mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=a Tm-increasing nucleotide, [+Xs]=a Tm-increasing nucleotide with a phosphorothioate linkage to the neighboring nucleotide, and [ademXs-L]=Lipid attached to a nucleotide with a phosphorothioate linkage to the neighboring nucleotide, wherein the antisense strand comprises a 5′ overhang of 2 nucleotides and a 3′ overhang of 7 nucleotides.

In some embodiments, a lipid-conjugated RNAi oligonucleotide for reducing expression of a target gene comprises the modification pattern of

Sense Strand:
5′- [+Xs][ademX-L][fX][fX][fX][fX][X][mX][mX][+X]
[+Xs][mXs][mX]-3′

### Hybridized to:

Antisense Strand:
5′ - [MePhosphonate-4O-mXs][fXs][fXs][fX][fX][mX]
[fX][mX][mX][fX][mX][mX][mX][fX][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=a Tm-increasing nucleotide, [+Xs]=a Tm-increasing nucleotide with a phosphorothioate linkage to the neighboring nucleotide, and [ademXs-L]=Lipid attached to a nucleotide with a phosphorothioate linkage to the neighboring nucleotide, wherein the antisense strand comprises a 5′ overhang of 2 nucleotides and a 3′ overhang of 7 nucleotides.

In some embodiments, a lipid-conjugated RNAi oligonucleotide for reducing expression of a target gene comprises the modification pattern of

Sense Strand:
5′- [ademXs-L][+X][fX][X][fX][mX][mX][mX][+X][+Xs]
[mXs][mX]-3′

### Hybridized to:

Antisense Strand:
5′ - [MePhosphonate-4O-mXs][fXs][fXs][fX][fX][mX]
[fX][mX][mX][fX][mX][mX][mXs][fXs][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=a Tm-increasing nucleotide, [+Xs]=a Tm-increasing nucleotide with a phosphorothioate linkage to the neighboring nucleotide, and [ademXs-L]=Lipid attached to a nucleotide with a phosphorothioate linkage to the neighboring nucleotide, wherein the antisense strand comprises a 5′ overhang of 2 nucleotides and a 3′ overhang of 8 nucleotides.

In some embodiments, a lipid-conjugated RNAi oligonucleotide for reducing expression of a target gene comprises the modification pattern of

Sense Strand:
5′- [ademXs-L][+X][fX][fX][fX][fX][mX][mX][mX]
[+Xs][+Xs][mX]-3′

### Hybridized to:

Antisense Strand:
5′ - [MePhosphonate-4O-mXs][fXs][fXs][fX][fX][mX]
[fX][X][mX][fX][mX][mX][mXs][fXs][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=a Tm-increasing nucleotide, [+Xs]=a Tm-increasing nucleotide with a phosphorothioate linkage to the neighboring nucleotide, and [ademXs-L]=Lipid attached to a nucleotide with a phosphorothioate linkage to the neighboring nucleotide, wherein the antisense strand comprises a 5′ overhang of 3 nucleotides and a 3′ overhang of 7 nucleotides.

In some embodiments, a lipid-conjugated RNAi oligonucleotide for reducing expression of a target gene comprises the modification pattern of

Sense Strand:
5′- [ademXs-L][+X][mX][mX][mX][+X][+X][mXs][mXs]
[mX]-3′

### Hybridized to:

Antisense Strand:
5′ - [MePhosphonate-4O-mXs][fXs][fXs][fX][fX][mX]
[fX][mX][mX][fX][mX][mX][mXs][fXs][mX][mX][mX][mX]
[mX][mXs][mXs][mX] -3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=Tm-increasing nucleotide, and [ademXs-L]=Lipid attached to a nucleotide with a phosphorothioate linkage to the neighboring nucleotide, wherein the antisense strand comprises a 5′ overhang of 1 nucleotide and a 3′ overhang of 11 nucleotides.

In some embodiments, a lipid-conjugated RNAi oligonucleotide for reducing expression of a target gene comprises the modification pattern as set forth in any one of Compounds 2-14 as described herein. In some embodiments, a lipid-conjugated RNAi oligonucleotide for reducing expression of a target gene comprises the modification pattern as set forth in Compound 15 as described herein. In some embodiments, a lipid-conjugated RNAi oligonucleotide for reducing expression of a target gene comprises the modification pattern as set forth in any one of Compounds 16-18 as described herein. In some embodiments, a lipid-conjugated RNAi oligonucleotide for reducing expression of a target gene comprises the modification pattern as set forth in any one of Compounds 19-38 as described herein. In some embodiments, a lipid-conjugated RNAi oligonucleotide for reducing expression of a target gene comprises the modification pattern as set forth in any one of Compounds 39-40, 45, and 48-49 as described herein. In some embodiments, a lipid-conjugated RNAi oligonucleotide for reducing expression of a target gene comprises the modification pattern as set forth in any one of Compounds 41-44 as described herein. In some embodiments, a lipid-conjugated RNAi oligonucleotide for reducing expression of a target gene comprises the modification pattern as set forth in any one of Compounds 46-47 as described herein. In some embodiments, a lipid-conjugated RNAi oligonucleotide for reducing expression of a target gene comprises the modification pattern as set forth in any one of Compounds 57 and 98-108 as described herein. In some embodiments, a lipid-conjugated RNAi oligonucleotide for reducing expression of a target gene comprises the modification pattern as set forth in any one of Compounds 120-122 as described herein. In some embodiments, a lipid-conjugated RNAi oligonucleotide for reducing expression of a target gene comprises the modification pattern as set forth in any one of Compounds 59-64 as described herein. In some embodiments, a lipid-conjugated RNAi oligonucleotide for reducing expression of a target gene comprises the modification pattern as set forth in any one of Compounds 65-69 as described herein. In some embodiments, a lipid-conjugated RNAi oligonucleotide for reducing expression of a target gene comprises the modification pattern as set forth in any one of Compounds 70-71 as described herein. In some embodiments, a lipid-conjugated RNAi oligonucleotide for reducing expression of a target gene comprises the modification pattern as set forth in any one of Compounds 72-74 as described herein. In some embodiments, a lipid-conjugated RNAi oligonucleotide for reducing expression of a target gene comprises the modification pattern as set forth in any one of Compounds 75-76 as described herein. In some embodiments, a lipid-conjugated RNAi oligonucleotide for reducing expression of a target gene comprises the modification pattern as set forth in any one of Compounds 77-79 as described herein. In some embodiments, a lipid-conjugated RNAi oligonucleotide for reducing expression of a target gene comprises the modification pattern as set forth in Compound 58 as described herein. In some embodiments, a lipid-conjugated RNAi oligonucleotide for reducing expression of a target gene comprises the modification pattern as set forth in any one of Compounds 80-81 as described herein. In some embodiments, a lipid-conjugated RNAi oligonucleotide for reducing expression of a target gene comprises the modification pattern as set forth in any one of Compounds 82-84 as described herein. In some embodiments, a lipid-conjugated RNAi oligonucleotide for reducing expression of a target gene comprises the modification pattern as set forth in any one of Compounds 85-86 as described herein. In some embodiments, a lipid-conjugated RNAi oligonucleotide for reducing expression of a target gene comprises the modification pattern as set forth in any one of Compounds 87-89 as described herein. In some embodiments, a lipid-conjugated RNAi oligonucleotide for reducing expression of a target gene comprises the modification pattern as set forth in any one of Compounds 90-91 as described herein. In some embodiments, a lipid-conjugated RNAi oligonucleotide for reducing expression of a target gene comprises the modification pattern as set forth in any one of Compounds 92-94 as described herein. In some embodiments, a lipid-conjugated RNAi oligonucleotide for reducing expression of a target gene comprises the modification pattern as set forth in Compound 95 as described herein. In some embodiments, a lipid-conjugated RNAi oligonucleotide for reducing expression of a target gene comprises the modification pattern as set forth in any one of Compounds 96-97 as described herein. In some embodiments, a lipid-conjugated RNAi oligonucleotide for reducing expression of a target gene comprises the modification pattern as set forth in any one of Compounds 109-117 as described herein. In some embodiments, a lipid-conjugated RNAi oligonucleotide for reducing expression of a target gene comprises the modification pattern as set forth in any one of Compounds 123-126 as described herein. In some embodiments, a lipid-conjugated RNAi oligonucleotide for reducing expression of a target gene comprises the modification pattern as set forth in any one of 127-130 as described herein. In some embodiments, a lipid-conjugated RNAi oligonucleotide for reducing expression of a target gene comprises the modification pattern as set forth in any one of Compounds 131-136 as described herein. In some embodiments, a lipid-conjugated RNAi oligonucleotide for reducing expression of a target gene comprises the modification pattern as set forth in any one of Compounds 137-140 as described herein. In some embodiments, a lipid-conjugated RNAi oligonucleotide for reducing expression of a target gene comprises the modification pattern as set forth in any one of Compounds 141-145 as described herein. In some embodiments, a lipid-conjugated RNAi oligonucleotide for reducing expression of a target gene comprises the modification pattern as set forth in any one of Compounds 146-148 as described herein. In some embodiments, a lipid-conjugated RNAi oligonucleotide for reducing expression of a target gene comprises the modification pattern as set forth in any one of Compounds 149-154 as described herein. In some embodiments, a lipid-conjugated RNAi oligonucleotide for reducing expression of a target gene comprises the modification pattern as set forth in any one of Compounds 155-160 as described herein. In some embodiments, a lipid-conjugated RNAi oligonucleotide for reducing expression of a target gene comprises the modification pattern as set forth in Compound 161 as described herein. In some embodiments, a lipid-conjugated RNAi oligonucleotide for reducing expression of a target gene comprises the modification pattern as set forth in any one of Compounds 162-165 and 171 as described herein. In some embodiments, a lipid-conjugated RNAi oligonucleotide for reducing expression of a target gene comprises the modification pattern as set forth in any one of Compounds 166-170 as described herein. In some embodiments, a lipid-conjugated RNAi oligonucleotide for reducing expression of a target gene comprises the modification pattern as set forth in Compound 172 as described herein. In some embodiments, a lipid-conjugated RNAi oligonucleotide for reducing expression of a target gene comprises the modification pattern as set forth in any one of Compounds 173-176 and 182 as described herein. In some embodiments, a lipid-conjugated RNAi oligonucleotide for reducing expression of a target gene comprises the modification pattern as set forth in any one of Compounds 177-181 as described herein. In some embodiments, a lipid-conjugated RNAi oligonucleotide for reducing expression of a target gene comprises the modification pattern as set forth in any one of 183-190 as described herein. In some embodiments, a lipid-conjugated RNAi oligonucleotide for reducing expression of a target gene comprises the modification pattern as set forth in any one of Compounds 191-194 as described herein. In some embodiments, a lipid-conjugated RNAi oligonucleotide for reducing expression of a target gene comprises the modification pattern as set forth in any one of Compounds 195-197 as described herein. In some embodiments, a lipid-conjugated RNAi oligonucleotide for reducing expression of a target gene comprises the modification pattern as set forth in any one of Compounds 200-201 as described herein. In some embodiments, a lipid-conjugated RNAi oligonucleotide for reducing expression of a target gene comprises the modification pattern as set forth in any one of Compounds 202-205, 207-210, and 211-215 as described herein. In some embodiments, a lipid-conjugated RNAi oligonucleotide for reducing expression of a target gene comprises the modification pattern as set forth in any one of Compounds 217-218 as described herein. In some embodiments, a lipid-conjugated RNAi oligonucleotide for reducing expression of a target gene comprises the modification pattern as set forth in any one of Compounds 219-222 and 224-232 as described herein. In some embodiments, a lipid-conjugated RNAi oligonucleotide for reducing expression of a target gene comprises the modification pattern as set forth in any one of 277-278 as described herein. In some embodiments, a lipid-conjugated RNAi oligonucleotide for reducing expression of a target gene comprises the modification pattern as set forth in any one of Compounds 279-285 as described herein. In some embodiments, a lipid-conjugated RNAi oligonucleotide for reducing expression of a target gene comprises the modification pattern as set forth in any one of Compounds 198-199 and 233 as described herein. In some embodiments, a lipid-conjugated RNAi oligonucleotide for reducing expression of a target gene comprises the modification pattern as set forth in any one of Compounds 234-236 as described herein. In some embodiments, a lipid-conjugated RNAi oligonucleotide for reducing expression of a target gene comprises the modification pattern as set forth in any one of Compounds 237-239 as described herein. In some embodiments, a lipid-conjugated RNAi oligonucleotide for reducing expression of a target gene comprises the modification pattern as set forth in any one of Compounds 240-242 as described herein. In some embodiments, a lipid-conjugated RNAi oligonucleotide for reducing expression of a target gene comprises the modification pattern as set forth in any one of Compounds 243-245 as described herein. In some embodiments, a lipid-conjugated RNAi oligonucleotide for reducing expression of a target gene comprises the modification pattern as set forth in any one of Compounds 246-251 as described herein. In some embodiments, a lipid-conjugated RNAi oligonucleotide for reducing expression of a target gene comprises the modification pattern as set forth in any one of Compounds 252-255 as described herein. In some embodiments, a lipid-conjugated RNAi oligonucleotide for reducing expression of a target gene comprises the modification pattern as set forth in Compound 256 as described herein. In some embodiments, a lipid-conjugated RNAi oligonucleotide for reducing expression of a target gene comprises the modification pattern as set forth in any one of Compounds 257-259 as described herein. In some embodiments, a lipid-conjugated RNAi oligonucleotide for reducing expression of a target gene comprises the modification pattern as set forth in any one of Compounds 260-262 as described herein. In some embodiments, a lipid-conjugated RNAi oligonucleotide for reducing expression of a target gene comprises the modification pattern as set forth in any one of Compounds 152 and 264-265 as described herein. In some embodiments, a lipid-conjugated RNAi oligonucleotide for reducing expression of a target gene comprises the modification pattern as set forth in any one of Compounds 266-268 as described herein. In some embodiments, a lipid-conjugated RNAi oligonucleotide for reducing expression of a target gene comprises the modification pattern as set forth in any one of Compounds 269-272 as described herein. In some embodiments, a lipid-conjugated RNAi oligonucleotide for reducing expression of a target gene comprises the modification pattern as set forth in any one of Compounds 273-276 as described herein. In some embodiments, a lipid-conjugated RNAi oligonucleotide for reducing expression of a target gene comprises the modification pattern as set forth in any one of Compounds 286 and 292 as described herein. In some embodiments, a lipid-conjugated RNAi oligonucleotide for reducing expression of a target gene comprises the modification pattern as set forth in any one of Compounds 287-291 as described herein. In some embodiments, a lipid-conjugated RNAi oligonucleotide for reducing expression of a target gene comprises the modification pattern as set forth in any one of Compounds 293-294 as described herein. In some embodiments, a lipid-conjugated RNAi oligonucleotide for reducing expression of a target gene comprises the modification pattern as set forth in any one of Compounds 2-15, 46 and 47 as described herein. In some embodiments, a lipid-conjugated RNAi oligonucleotide for reducing expression of a target gene comprises the modification pattern as set forth in any one of Compounds 137-140. 146, 148, 162-165, 173-176, 183-190, 200, 217, 247, 270, 277, 278, 286 and 292 as described herein. In some embodiments, a lipid-conjugated RNAi oligonucleotide for reducing expression of a target gene comprises the modification pattern as set forth in any one of Compounds 16, 19-28, 30-38, 41-44, 65-79, 80-97, 127-130, 202-205, 207-210, 212-215, 219-221, 224-227, 229-232, 237-239, 243-245, 252-255, 260-262, 266-268, 274-276, 280-285, 287-291, and 293-294 as described herein. In some embodiments, a lipid-conjugated RNAi oligonucleotide for reducing expression of a target gene comprises the modification pattern as set forth in any one of Compounds 40, 45, 49, 59-64, 98, 99-117, 131-136, 141-145, 149-160, 166-170, 177-181, 191-197, 201, 211, 218, 228, 234-236, 240-242, 248-251, 257-259, 264, 265, 272-273, and 279 as described herein. In some or any of the foregoing embodiments, reference to Compound numbers refers to the modification pattern (e.g., phosphorothioate linkages, 2′ modifications, conjugation) and not the nucleotide sequences.

### General Methods of Providing the Nucleic Acids and Analogues Thereof

The nucleic acids and analogues thereof comprising lipid conjugate described herein can be made using a variety of synthetic methods known in the art, including standard phosphoramidite methods. Any phosphoramidite synthesis method can be used to synthesize the provided nucleic acids of this disclosure. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond there between, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a lipid conjugate moiety as described herein. Typically, the coupling, capping/oxidizing steps and optionally, the deprotecting steps, are repeated until the oligonucleotide reaches the desired length and/or sequence, after which it is cleaved from the solid support. In certain embodiments, an oligonucleotide is prepared comprising 1-3 nucleic acid or analogues thereof comprising lipid conjugates units on a tetraloop.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in MARCH'S ADVANCED ORGANIC CHEMISTRY: REACTIONS, MECHANISMS, AND STRUCTURE, M. B. Smith and J. March, 5^{th} Edition, John Wiley & Sons, 2001, COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids, and analogues thereof of the present disclosure are generally prepared according to Scheme A, Scheme A1 and Scheme B set forth below:

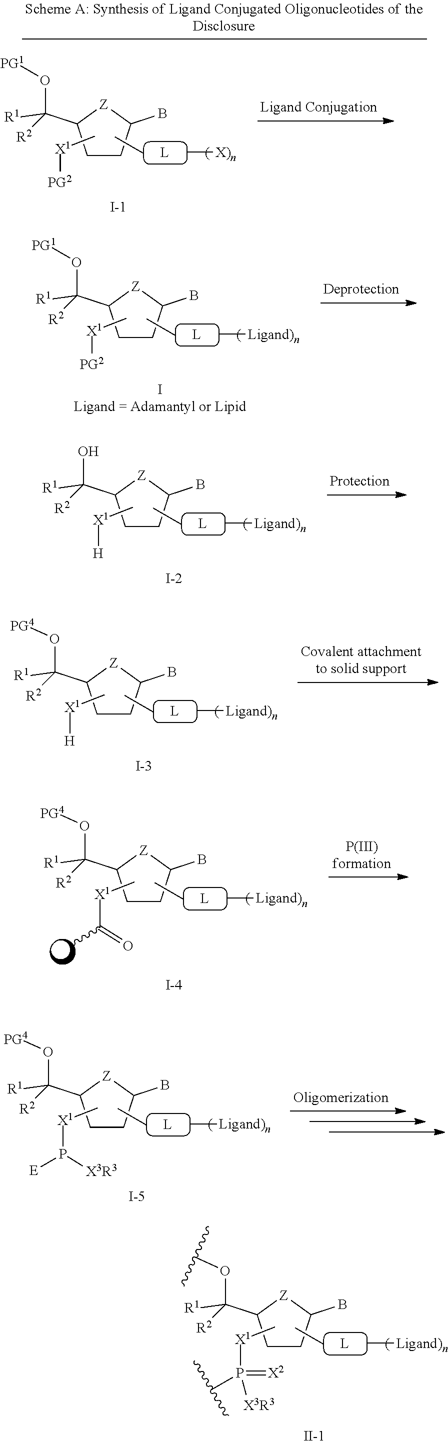

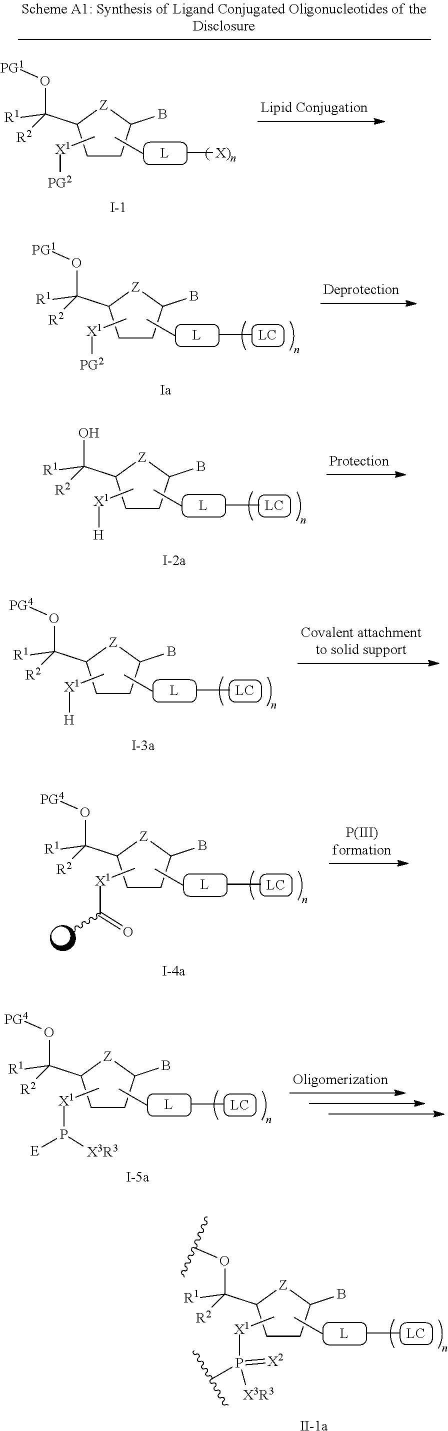

As depicted in Scheme A and Scheme A1 above, a nucleic acid or analogue thereof of formula I-1 is conjugated with one or more ligand/lipophilic compound to form a compound of formula I or Ia comprising one more ligand/lipid conjugates. Typically, conjugation is performed through an esterification or amidation reaction between a nucleic acid or analogue thereof of formula I-1 or I-1a and one or more adamantyl and/or lipophilic compound (e.g., fatty acid) in series or in parallel by known techniques in the art. Nucleic acid or analogue thereof of formula I or Ia can then be deprotected to form a compound of formula I-2 or I-2a and protected with a suitable hydroxyl protecting group (e.g., DMTr) to form a compound of formula I-3 or I-3a. In one aspect, nucleic acid-ligand conjugates of formula I-3 or I-3a can be covalently attached to a solid support (e.g., through a succinic acid linking group) to form a solid support nucleic acid-ligand conjugate or analogue thereof of formula I-4 or I-4a comprising one or more adamantyl and/or lipid conjugate. In another aspect, a nucleic acid-ligand conjugates of formula 1-3 or I-3a can react with a P(III) forming reagent (e.g., 2-cyanoethyl N,N-di-isopropylchlorophosphoramidite) to form a nucleic acid or analogue thereof of formula I-5 or I-5a comprising a P(III) group. A nucleic acid-ligand conjugate or analogue thereof of formula I-5 or I-5a can then be subjected to oligomerization forming conditions preformed using known and commonly applied processes to prepare oligonucleotides in the art. For example, the compound of formula I-5 or I-5a is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more lipid conjugate nucleotide units represented by a compound of formula II-1 or II-Ia. Each of B, E, L, ligand, LC, n, PG¹, PG², PG⁴, R¹, R², R³, X, X¹, X², X³, and Z is as defined above and described herein.

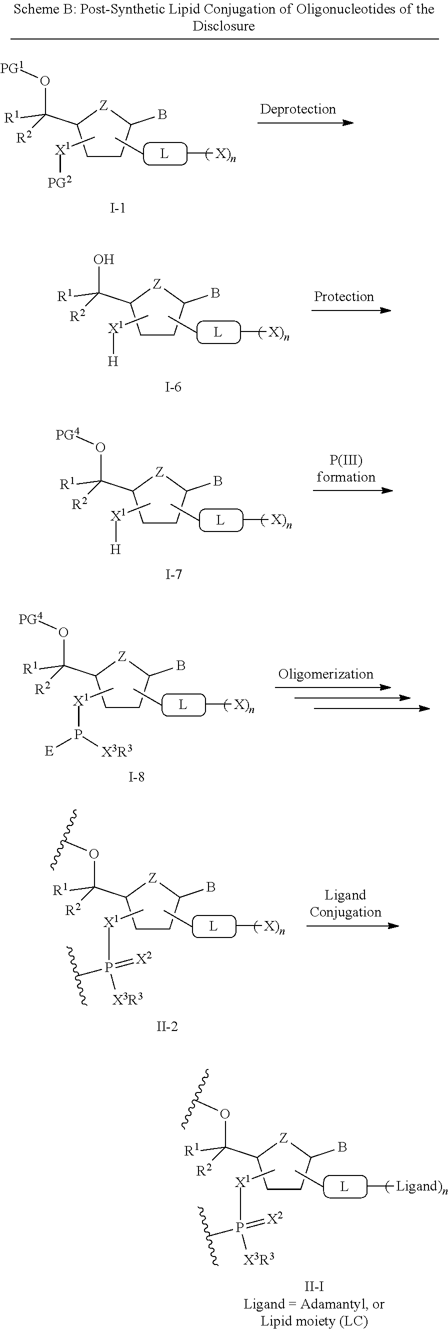

As depicted in Scheme B above, a nucleic acid or analogue thereof of formula I-1 can be deprotected to form a compound of formula I-6, protected with a suitable hydroxyl protecting group (e.g., DMTr) to form a compound of formula I-7, and reacted with a P(III) forming reagent (e.g., 2-cyanoethyl N,N-di-isopropylchlorophosphoramidite) to form a nucleic acid or analogue thereof of formula 1-8 comprising a P(III) group. Next, a nucleic acid or analogue thereof of formula I-8 is subjected to oligomerization forming conditions preformed using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths represented by a compound of formula II-2. An oligonucleotide of formula II-2 can then be conjugated with one or more ligands e.g., adamantyl, or lipophilic compound (e.g., fatty acid) to form a compound of formula II-1 comprising one or more ligand conjugates. Typically, conjugation is performed through an esterification or amidation reaction between a nucleic acid or analogue thereof of formula II-2 and one or more adamantyl or fatty acid in series or in parallel by known techniques in the art. Each of B, E, L, ligand, LC, n, PG¹, PG², PG⁴, R¹, R², R³, X, X¹, X², X³, and Z is as defined above and described herein.

In certain embodiments, nucleic acids, and analogues thereof of the present disclosure are prepared according to Scheme C and Scheme D set forth below:

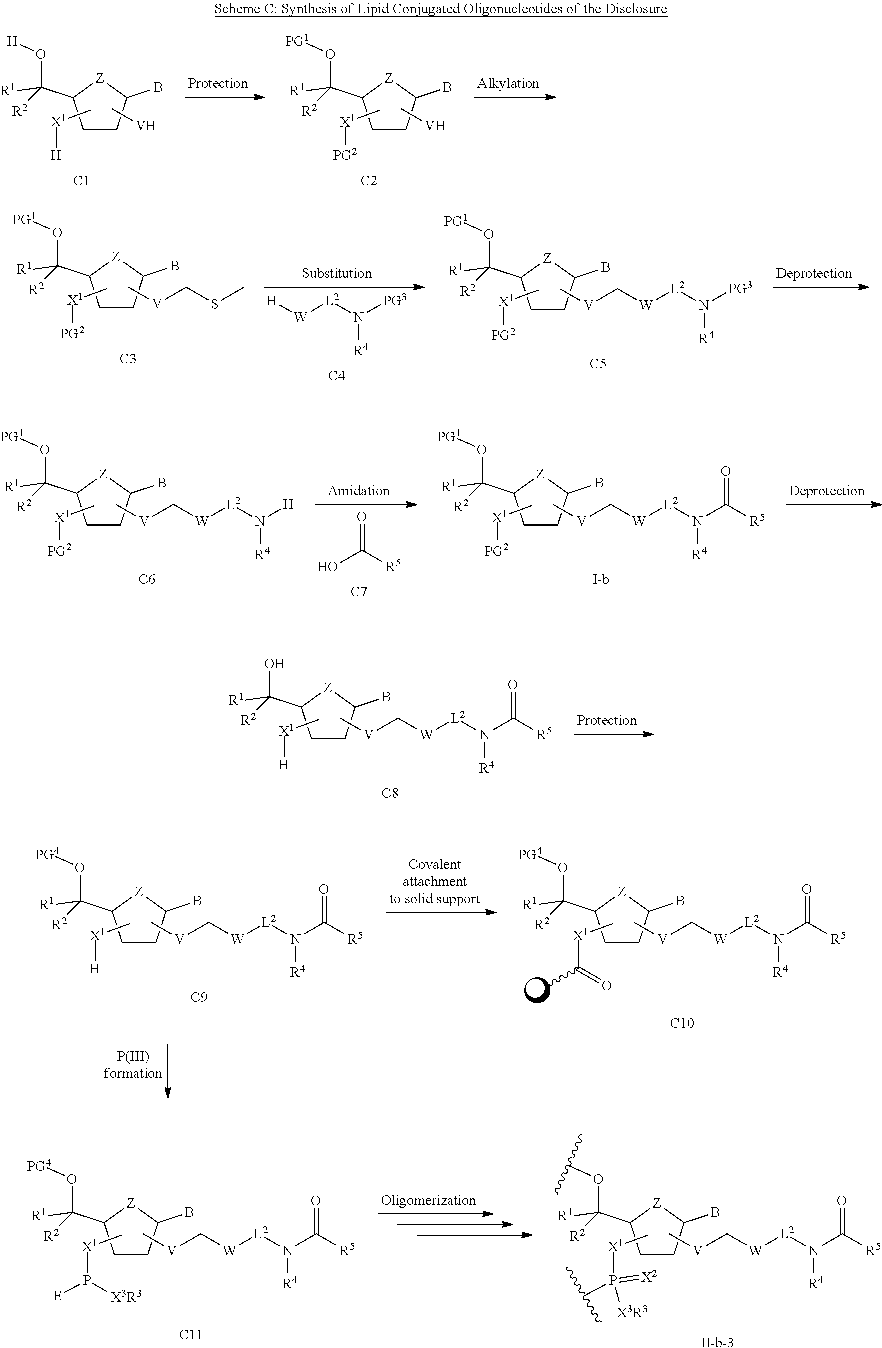

As depicted in Scheme C above, a nucleic acid or analogue thereof of formula C1 is protected to form a compound of formula C2. Nucleic acid or analogue thereof of formula C2 is then alkylated (e.g., using DMSO and acetic acid via the Pummerer rearrangement) to form a monothioacetal compound of formula C3. Next, nucleic acid or analogue thereof of formula C3 is coupled with C4 under appropriate conditions (e.g., mild oxidizing conditions) to form a nucleic acid or analogue thereof of formula C5. Nucleic acid or analogue thereof of formula C5 can then be deprotected to form a compound of formula C6 and coupled with a ligand (adamantyl or lipophilic compound (e.g., a fatty acid)) of formula C7 under appropriate amide forming conditions (e.g., HATU, DIPEA), to form a nucleic acid-ligand conjugate or analogue thereof of formula I-b comprising a lipid conjugate of the disclosure. Nucleic acid-ligand conjugate or analogue thereof of formula I-b can then be deprotected to form a compound of formula C8 and protected with a suitable hydroxyl protecting group (e.g., DMTr) to form a compound of formula C9. In one aspect, nucleic acid, or analogue thereof of formula C9 can be covalently attached to a solid support (e.g., through a succinic acid linking group) to form a solid support nucleic acid-ligand conjugate or analogue thereof of formula C10 comprising a ligand conjugate (adamantyl or lipid moiety) of the disclosure. In another aspect, a nucleic acid-ligand conjugate or analogue thereof of formula C9 can reacted with a P(III) forming reagent (e.g., 2-cyanoethyl N,N-di-isopropylchlorophosphoramidite) to form a nucleic acid-ligand conjugate or analogue thereof of formula C11 comprising a P(III) group. A nucleic acid-ligand conjugate or analogue thereof of formula C11 can then be subjected to oligomerization forming conditions preformed using known and commonly applied processes to prepare oligonucleotides in the art. For example, the compound of formula C11 is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more adamantyl and/or lipid conjugate nucleotide units represented by a compound of formula II-b-3. Each of B, E, L², PG¹, PG², PG³, PG⁴, R¹, R², R³, R⁴, R⁵, X¹, X², X³, V, W, and Z is as defined above and described herein.

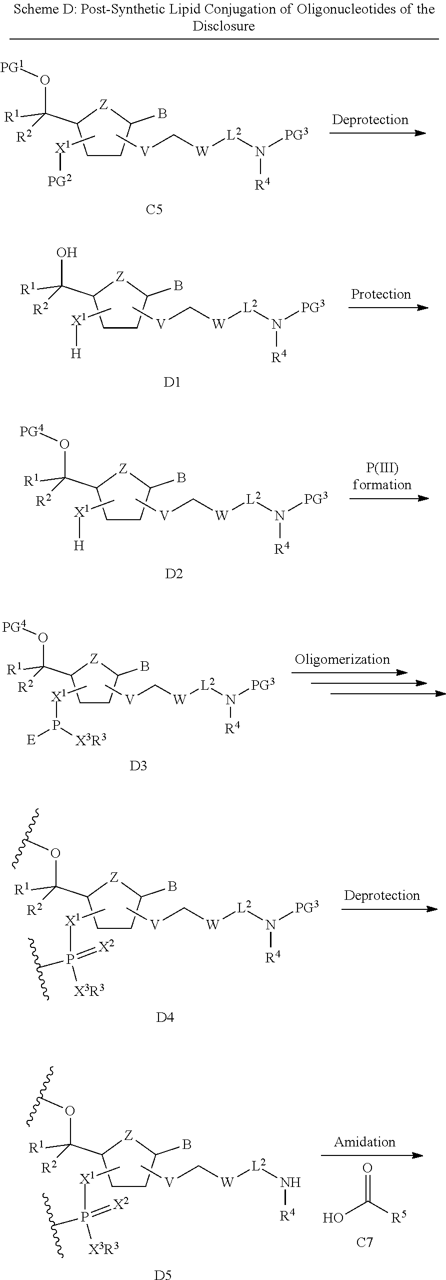

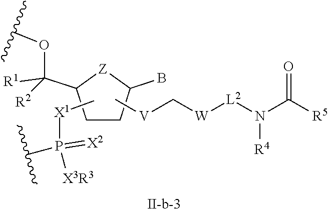

Each of B, E, L², PG¹, PG², PG³, PG⁴, R¹, R², R³, R⁴, R⁵, X¹, X², X³, V, W, and Z is as defined above and described herein. As depicted in Scheme D above, a nucleic acid or analogue thereof of formula C5 can be selectively deprotected to form a compound of formula D1, protected with a suitable hydroxyl protecting group (e.g., DMTr) to form a compound of formula D2, and reacted with a P(III) forming reagent (e.g., 2-cyanoethyl N,N-di-isopropylchlorophosphoramidite) to form a nucleic acid or analogue thereof of formula D3. Next, a nucleic acid or analogue thereof of formula D3 is subjected to oligomerization forming conditions preformed using known and commonly applied processes to prepare oligonucleotides in the art. For example, the compound of formula D3 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths, represented by a compound of formula D4. An oligonucleotide of formula D4 can then be deprotected to form a compound of formula D5 and coupled with a hydrophobic ligand (e.g., adamantyl or a lipophilic moiety) to form a compound of formula C7 (e.g., adamantyl or a fatty acid) under appropriate amide forming conditions (e.g., HATU, DIPEA), to form an oligonucleotide of formula II-b-3 comprising a ligand (e.g., adamantyl or a fatty acid) conjugate of the disclosure.

One of skill in the art will appreciate that various functional groups present in the nucleic acid or analogues thereof of the disclosure such as aliphatic groups, alcohols, carboxylic acids, esters, amides, aldehydes, halogens, and nitriles can be interconverted by techniques well known in the art including, but not limited to reduction, oxidation, esterification, hydrolysis, partial oxidation, partial reduction, halogenation, dehydration, partial hydration, and hydration. See for example, “MARCH'S ADVANCED ORGANIC CHEMISTRY”, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleic acids of the disclosure are described below in the Exemplification.

In some embodiments, the present disclosure provides a method for preparing an oligonucleotide comprising one or more lipid conjugate, said lipid conjugate unit represent by formula II-a-1:

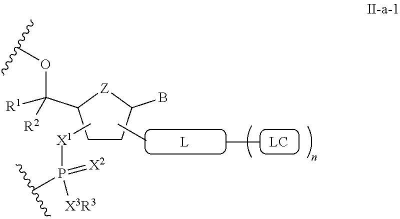

or a pharmaceutically acceptable salt thereof, comprising the steps of:
(a) providing a nucleic acid or analogue thereof of formula I-5a:

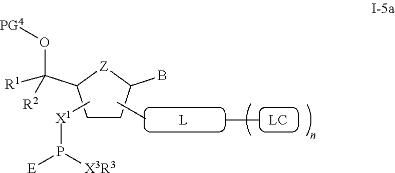

or salt thereof, and
(b) oligomerizing said compound of formula I-5a to form a compound of formula II-1a, wherein each of B, E, L, LC, n, PG⁴, R¹, R², R³, X, X¹, X², X³, E, and Z is as defined above and described herein.

In step (b) above, oligomerizing refers to preforming oligomerization forming conditions using known and commonly applied processes to prepare oligonucleotides in the art. For example, the compound of formula I-5a is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-1a comprising a lipid conjugate of the disclosure.

In some embodiments, the present disclosure provides a method for preparing an oligonucleotide comprising one or more lipid conjugate, further comprising preparing a nucleic acid or analogue thereof of formula I-5a:

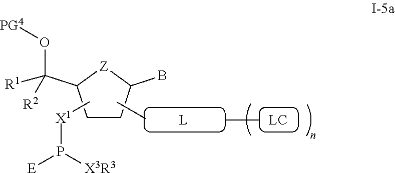

or a salt thereof, comprising the steps of:
(a) providing a nucleic acid or analogue thereof of formula Ia:

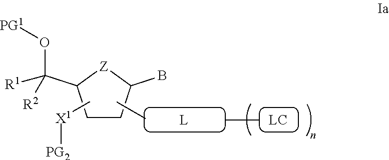

or salt thereof,
(b) deprotecting said nucleic acid or analogue thereof of formula Ia to form a compound of formula I-2a:

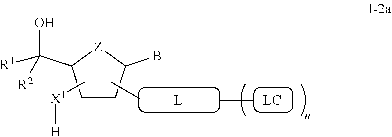

or salt thereof,
(c) protecting said nucleic acid or analogue thereof of formula I-2 to form a compound of formula I-3a:

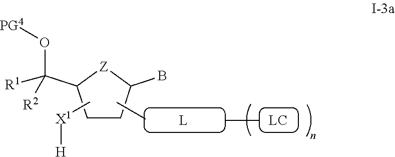

or salt thereof, and
(d) treating said nucleic acid or analogue thereof of formula I-3a with a P(III) forming reagent to form a nucleic acid or analogue thereof of formula I-5a, wherein each of B, E, L, LC, n, PG⁴, R¹, R², R³, X, X¹, X², X³, E, and Z is as defined above and described herein.

In step (b) above, PG¹ and PG² of a compound of formula Ia comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-2a is protected with a suitable hydroxyl protecting group. In certain embodiments, the protecting group PG⁴ used for protection of the 5′-hydroxyl group of a compound of formula I-2a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-3a is treated with a P(III) forming reagent to afford a compound of formula I-5a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-3a is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide comprising one or more lipid conjugates, further comprising preparing a nucleic acid-lipid conjugate or analogue thereof of formula Ia:

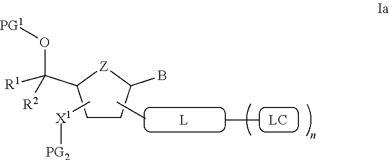

or a salt thereof, comprising the steps of:
(a) providing a nucleic acid or analogue thereof of formula I-1:

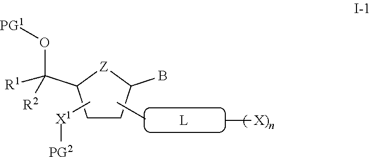

or salt thereof, and,
(b) conjugating one or more lipophilic compounds to a nucleic acid or analogue thereof of formula I-1 to form a nucleic acid or analogue thereof of formula Ia comprising one or more lipid conjugates, wherein: each of B, E, L, LC, n, PG¹, PG², R¹, R², X, X¹, and Z is as defined above and described herein.

In step (b) above, a nucleic acid or analogue thereof of formula I-1a is conjugated with one or more lipophilic compounds to form a compound of formula Ia comprising one more lipid conjugates of the disclosure. Typically, conjugation is performed through an esterification or amidation reaction between a nucleic acid or analogue thereof of formula I-1a and one or more fatty acids in series or in parallel by known techniques in the art. In certain embodiments, conjugation is performed under suitable amide forming conditions to afford a compound of formula I comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising one or more lipid conjugate, said lipid conjugate unit represent by formula II-1:

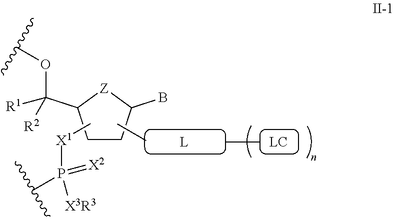

or a pharmaceutically acceptable salt thereof, comprising the steps of:
(a) providing an oligonucleotide of formula II-2:

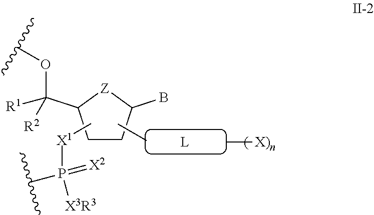

or salt thereof, and,
(b) conjugating one or more lipophilic compounds to an oligonucleotide of formula II-2 to form an oligonucleotide of formula II-1 comprising one or more lipid conjugates. In step (b) above, an oligonucleotide of formula II-2 is conjugated with one or more lipophilic compounds to form an oligonucleotide of formula II-1 comprising one more lipid conjugates of the disclosure. Typically, conjugation is performed through an esterification or amidation reaction between an oligonucleotide of formula II-2 and one or more fatty acids in series or in parallel by known techniques in the art. In certain embodiments, conjugation is performed under suitable amide forming conditions to afford an oligonucleotide of formula II-1 comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising a unit represent by formula II-2:

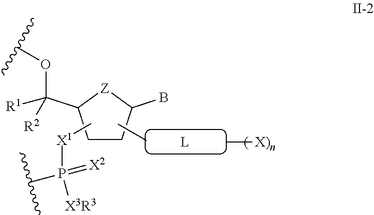

or a pharmaceutically acceptable salt thereof, comprising the steps of:
(a) providing a nucleic acid or analogue thereof of formula I-8:

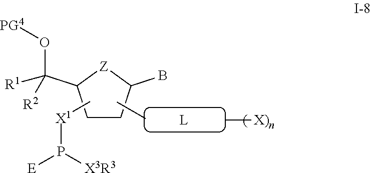

or salt thereof, and
(b) oligomerizing said compound of formula I-8 to form a compound of formula II-2.

In step (b) above, oligomerizing refers to preforming oligomerization forming conditions using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-2.

In some embodiments, the present disclosure provides a method for preparing a nucleic acid or analogue thereof comprising one or more lipid conjugate, further comprising preparing a nucleic acid or analogue thereof of formula I-8:

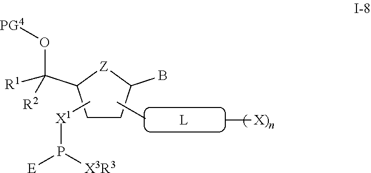

or a salt thereof, comprising the steps of:
(a) providing a nucleic acid or analogue thereof of formula I-1:

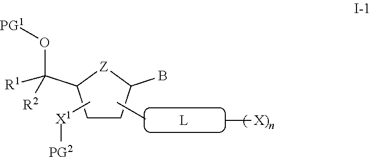

or salt thereof,
(b) deprotecting said nucleic acid or analogue thereof of formula I-1 to form a compound of formula I-6:

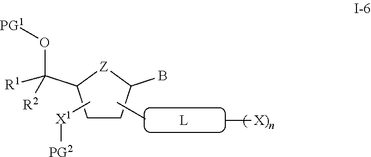

or salt thereof,
(c) protecting said nucleic acid or analogue thereof of formula I-6 to form a compound of formula I-7:

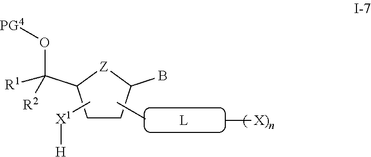

or salt thereof, and
(d) treating said nucleic acid or analogue thereof of formula I-7 with a P(III) forming reagent to form a nucleic acid or analogue thereof of formula I-8, In step (b) above, PG¹ and PG² of a compound of formula I-1 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-6 is protected with a suitable hydroxyl protecting group. In certain embodiments, the protecting group PG⁴ used for protection of the 5′-hydroxyl group of a compound of formula I-6 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-7 is treated with a P(III) forming reagent to afford a compound of formula I-8. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-7 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate comprising one or more adamantyl and/or lipid moieties, said conjugate unit represented by formula II-b-3:

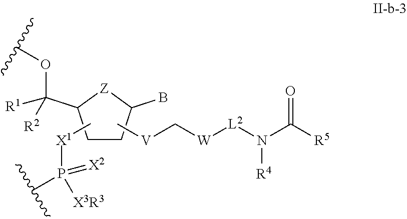

or a pharmaceutically acceptable salt thereof, comprising the steps of:
(a) providing a nucleic acid-ligand conjugate or analogue thereof of formula C11:

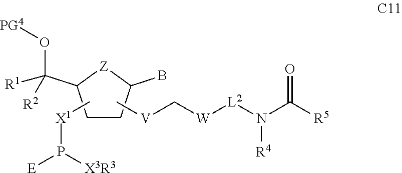

or salt thereof, and
(b) oligomerizing said compound of formula C11 to form a compound of formula II-b-3, In step (b) above, oligomerizing refers to preforming oligomerization forming conditions using known and commonly applied processes to prepare oligonucleotides in the art. For example, the compound of formula C11 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide-ligand conjugate of various nucleotide lengths, with one or more nucleic acid-ligand conjugate units, wherein each unit is represented by a compound of formula II-b-3 comprising an adamantyl or lipid moiety of the disclosure.

In some embodiments, the method for preparing an oligonucleotide of formula II-b-3 comprising one or more lipid conjugate, further comprises preparing a nucleic acid-ligand conjugate or analogue thereof of formula C11:

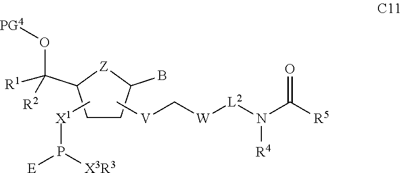

or a salt thereof, comprising the steps of:
(a) providing a nucleic acid-ligand conjugate or analogue thereof of formula I-b:

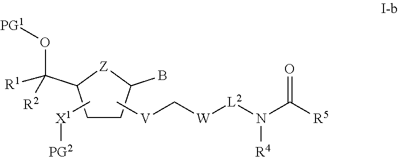

or salt thereof,
(b) deprotecting said nucleic acid-ligand conjugate or analogue thereof of formula I-b to form a compound of formula C8:

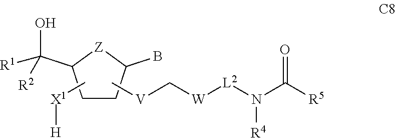

or salt thereof,
(c) protecting said nucleic acid-ligand conjugate or analogue thereof of formula C8 to form a compound of formula C9:

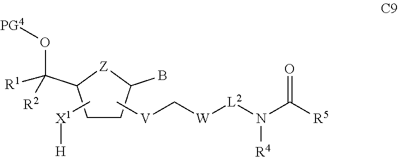

or salt thereof, and
(d) treating said nucleic acid-ligand conjugate or analogue thereof of formula C9 with a P(III) forming reagent to form a nucleic acid or analogue thereof of formula C11. In step (b) above, PG¹ and PG² of a compound of formula I-b comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula C8 is protected with a suitable hydroxyl protecting group. In certain embodiments, the protecting group PG⁴ used for protection of the 5′-hydroxyl group of a compound of formula C8 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula C9 is treated with a P(III) forming reagent to afford a compound of formula C11. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula C9 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate of formula II-b-3 comprising one or more nucleic acid-ligand conjugate units each comprising one or more adamantyl or lipid moieties, further comprising preparing a nucleic acid-ligand conjugate or analogue thereof of formula I-b:

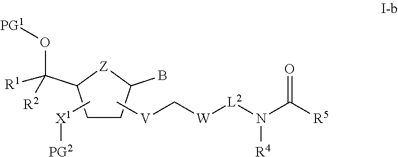

or a salt thereof, comprising the steps of:
(a) providing a nucleic acid-ligand conjugate or analogue thereof of formula C6:

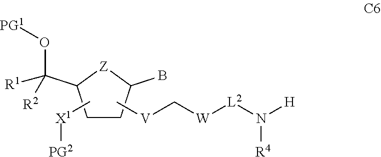

or salt thereof, and,
(b) conjugating a lipophilic compound to a nucleic acid or analogue thereof of formula C6 to form a nucleic acid-ligand conjugate or analogue thereof of formula I-b comprising one or more adamantyl and/or lipid conjugates. In step (b) above, conjugation is performed under suitable amide forming conditions to afford a compound of formula I-b comprising an adamantyl and/or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU. In certain embodiments, the amide forming conditions comprise HATU and DIPEA or TEA.

In certain embodiments, a nucleic acid-ligand conjugate or analogue thereof of formula C6 is provided in salt form (e.g., a fumarate salt) and is first converted to the free base (e.g., using sodium bicarbonate) before preforming the conjugation step.

In some embodiments, the present disclosure provides a method for preparing an oligonucleotide-ligand conjugate of formula II-b-3 comprising one or more nucleic acid-ligand conjugate units, further comprises preparing a nucleic acid-ligand conjugate or analogue thereof of formula C6:

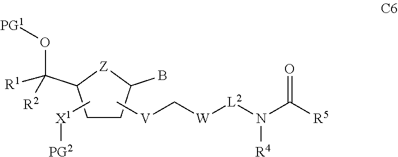

or a salt thereof, comprising the steps of:
(a) providing a nucleic acid or analogue thereof of formula C1:

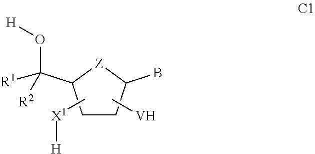

or salt thereof, and,
(b) protecting said nucleic acid or analogue thereof of formula C1 to form a compound of formula C2:

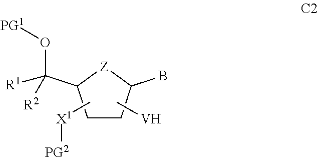

or salt thereof,
(c) alkylating said nucleic acid or analogue thereof of formula C2 to form a compound of formula C3:

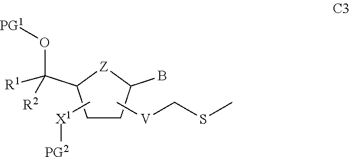

or salt thereof,
(d) substituting said nucleic acid or analogue thereof of formula C3 with a compound of formula C4:

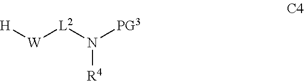

or salt thereof, to form a compound of formula C5:

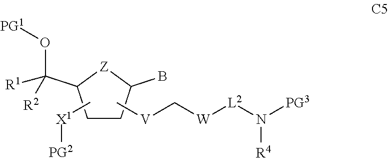

or salt thereof,
(e) deprotecting said nucleic acid or analogue thereof of formula C5 to form a nucleic acid-ligand conjugate or analogue thereof of formula C6. In step (b) above, PG¹ and PG² groups of formula C2 are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula C1 and 1,3-dichloro-1,1,3,3-tetraisopropyldisiloxane under basic conditions.

In step (c) above, a nucleic acid or analogue thereof of formula C2 is alkylated with a mixture of DMSO and acetic anhydride under acidic conditions. In certain embodiments, when —V—H is a hydroxyl group, the mixture of DMSO and acetic anhydride in the presence of acetic acid forms (methylthio)methyl acetate in situ via the Pummerer rearrangement which then reacts with the hydroxyl group of the nucleic acid or analogue thereof of formula C2 to provide a monothioacetal functionalized fragment nucleic acid or analogue thereof of formula C3.

In step (d) above, substitution of the thiomethyl group of a nucleic acid or analogue thereof of formula C3 using a nucleic acid or analogue thereof of formula C4 affords a nucleic acid or analogue thereof of formula C4. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In step (e) above, removal of PG³ and optionally R⁴ (when R⁴ is a suitable amine protecting group) of a nucleic acid-ligand conjugate or analogue thereof of formula C5 affords a nucleic acid-ligand conjugate or analogue thereof of formula C6 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of a nucleic acid-ligand conjugate or analogue thereof of formula C5 are removed by acid hydrolysis. It will be appreciated that upon acid hydrolysis of the protecting groups of a nucleic acid-ligand conjugate or analogue thereof of formula C5, a salt of formula C6 thereof is formed. For example, when an acid-labile protecting group of a nucleic acid-ligand conjugate or analogue thereof of formula C5 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid or analogue thereof of formula C6 are contemplated.

In other embodiments, the protecting groups (e.g., both PG³ and R⁴ or either of PG³ or R⁴ independently) of a nucleic acid or analogue thereof of formula C5 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU). In certain embodiments, a nucleic acid-ligand conjugate or analogue thereof of formula C5 is deprotected under basic conditions followed by treating with an acid to form a salt of formula C6. In certain embodiments, the acid is fumaric acid the salt of formula C6 is the fumarate.

In some embodiments, the present disclosure provides a method for preparing an oligonucleotide-ligand conjugate comprising one or more nucleic acid-ligand conjugate, said nucleic acid-ligand conjugate unit represented by formula II-b-3:

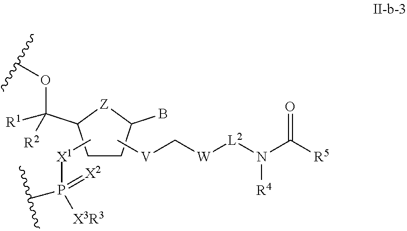

or a pharmaceutically acceptable salt thereof, comprising the steps of:
(a) providing an oligonucleotide of formula D5:

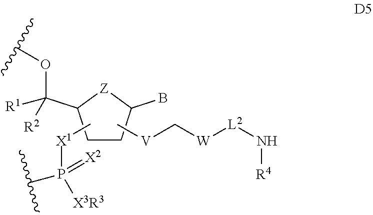

or salt thereof, and,
(b) conjugating one or more adamantyl or lipophilic compounds to an oligonucleotide of formula D5 to form an oligonucleotide-ligand conjugate of formula II-b-3 comprising one or more nucleic acid-ligand conjugate units. In step (b) above, conjugation is performed under suitable amide forming conditions to afford a compound of formula D5 comprising an adamantyl or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU. In certain embodiments, the amide forming conditions comprise HATU and DIPEA or TEA.

In some embodiments, the present disclosure provides a method for preparing an oligonucleotide-ligand conjugate comprising a unit represent by formula D5:

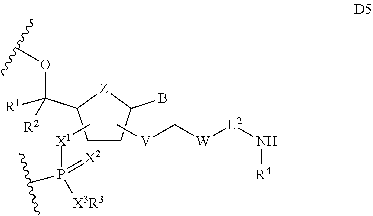

or a salt thereof, comprising the steps of:
(a) providing a nucleic acid-ligand conjugate or analogue thereof of formula D4:

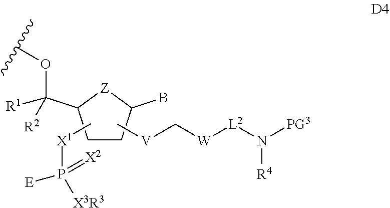

or salt thereof, and
(b) deprotecting said compound of formula D4 to form a compound of formula D5. In step (b) above, removal of PG³ and optionally R⁴ (when R⁴ is a suitable amine protecting group) of an oligonucleotide of formula D4 affords an oligonucleotide-ligand conjugate of formula D5 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of an oligonucleotide-ligand conjugate of formula D4 are removed by acid hydrolysis. It will be appreciated that upon acid hydrolysis of the protecting groups of an oligonucleotide-ligand conjugate of formula D4, a salt of formula D5 thereof is formed. For example, when an acid-labile protecting group of an oligonucleotide of formula D4 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid-ligand conjugate unit or analogue thereof of formula D5 are contemplated.

In other embodiments, the protecting groups (e.g., both PG³ and R⁴ or either of PG³ or R⁴ independently) of an oligonucleotide-ligand conjugate of formula D4 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU).

In some embodiments, the present disclosure provides a method for preparing an oligonucleotide-ligand conjugate comprising one or more nucleic acid-ligand conjugate unit with one or more adamantyl and/or lipid moiety, said conjugate unit represented by formula D4:

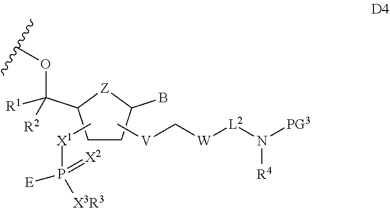

or a pharmaceutically acceptable salt thereof, comprising the steps of:
(a) providing a nucleic acid or analogue thereof of formula D3:

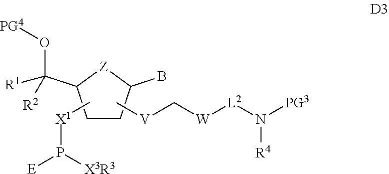

or salt thereof, and
(b) oligomerizing said compound of formula D3 to form a compound of formula D4,

In step (b) above, oligomerizing refers to preforming oligomerization forming conditions using known and commonly applied processes to prepare oligonucleotides in the art. For example, the nucleic acid or analogue thereof of formula D3 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula D4 comprising an adamantyl or lipid conjugate of the disclosure.

In some embodiments, the present disclosure provides a method for preparing a nucleic acid or analogue thereof comprising one or more lipid conjugate, further comprising preparing a nucleic acid or analogue thereof of formula D3:

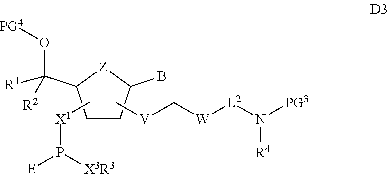

or a salt thereof, comprising the steps of:
(a) providing a nucleic acid or analogue thereof of formula C5:

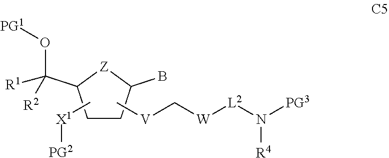

or salt thereof,
(b) deprotecting said nucleic acid or analogue thereof of formula C5 to form a compound of formula D1:

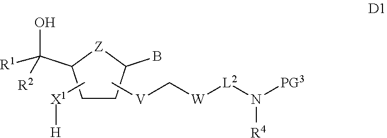

or salt thereof,
(c) protecting said nucleic acid or analogue thereof of formula D1 to form a nucleic acid or analogue thereof of formula D2:

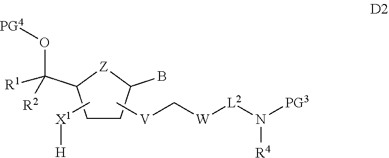

or salt thereof, and
(d) treating said nucleic acid or analogue thereof of formula D2 with a P(III) forming reagent to form a nucleic acid or analogue thereof of formula D3. In step (b) above, PG¹ and PG² of a nucleic acid or analogue thereof of formula C5 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a nucleic acid or analogue thereof of formula D1 is protected with a suitable hydroxyl protecting group. In certain embodiments, the protecting group PG⁴ used for protection of the 5′-hydroxyl group of a compound of formula D1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a nucleic acid or analogue thereof of formula D2 is treated with a P(III) forming reagent to afford a compound of formula D3. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanocthyl phosphorodichloridate. In certain embodiments, the P(III) forming reagent is 2-cyanocthyl N,N-diisopropylchlorophosphoramidite. One of ordinary skill would recognize that the displacement of a leaving group in a P(III) forming reagent by X¹ of a compound of formula D2 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

### Formulations

Various formulations have been developed to facilitate oligonucleotide use. For example, oligonucleotides (e.g., lipid-conjugated RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., lipid-conjugated RNAi oligonucleotides) reduce the expression of a target mRNA (e.g., a target mRNA expressed in an neurons of the CNS) . . . . In some embodiments, provided herein are compositions comprising oligonucleotides (e.g., lipid-conjugated RNAi oligonucleotides) reduce the expression of a target mRNA expressed in one or more tissues or cells of a subject. Such compositions can be suitably formulated such that when administered to a subject, either into the immediate environment of a target cell or systemically, a sufficient portion of the oligonucleotides enter the cell to reduce target gene expression. Any variety of suitable oligonucleotide formulations can be used to deliver oligonucleotides for the reduction of target gene expression as disclosed herein. In some embodiments, an oligonucleotide is formulated in buffer solutions such as phosphate buffered saline solutions, liposomes, micellar structures, and capsids.

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin). Likewise, the oligonucleotides herein may be provided in the form of their free acids.

In some embodiments, a pharmaceutical composition is formulated to be compatible with its intended route of administration. Examples of routes of administration include parenteral (e.g., intravenous, intramuscular, intraperitoneal, intradermal, subcutaneous, intrathecal), oral (e.g., inhalation), transdermal (e.g., topical), transmucosal and rectal administration. In some embodiments, a pharmaceutical composition is formulated for delivery to the central nervous system (e.g., intrathecal, epidural). In some embodiments, a pharmaceutical composition is formulated for delivery to the eye (e.g., ophthalmic, intraocular, subconjunctival, intravitreal, retrobulbar, intracameral).

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an lipid-conjugated RNAi oligonucleotide herein) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Structural Modifications

As nucleic acids are polymers of subunits or compounds, many of the modifications described below occur at a position which is repeated within a nucleic acid (e.g., a modification of a base, or a phosphate moiety, or the non-bridging oxygen of a phosphate moiety). In some cases the modification will occur at all of the subject positions in the nucleic acid but in many, and in fact in most cases it will not. By way of example, a modification may only occur at a 3′ or 5′ terminal position, may only occur in the internal unpaired region, may only occur in a terminal regions, e.g. at a position on a terminal nucleotide or in the last 2, 3, 4, 5, or 10 nucleotides of a strand. In some embodiments, a modification occurs at all of the subject positions in the nucleic acid. A modification may occur in a double strand region, a single strand region, or in both. A modification may occur only in the double strand region of an RNA agent or may only occur in a single strand region of an RNA agent. (e.g., a phosphorothioate modification at a non-bridging oxygen position may only occur at one or both termini, may only occur in a terminal regions or at a position on a terminal nucleotide or in the last 2, 3, 4, 5, or 10 nucleotides of a strand, or may occur in double strand and single strand regions, particularly at termini. The 5′ end or ends can be phosphorylated.

Many studies in the art have indicated that modified oligonucleotides and oligonucleotide analogs may be less readily internalized than their natural counterparts. As a result, the activity of many previously available RNAi trigger molecules has not been sufficient for practical therapeutic, research or diagnostic purposes.

Modifications to enhance the effectiveness of the RNAi trigger molecule oligonucleotides and overcome these problems have taken many forms. These modifications include base ring modifications, sugar moiety modifications, and sugar-phosphate backbone modifications, many exemplified herein and used in the current disclosure. Prior sugar-phosphate backbone modifications, particularly on the phosphorus atom, have affected various levels of resistance to nucleases. However, while the ability of an RNAi trigger molecule oligonucleotide to load into the RISC and direct the location of relevant mRNA sequences is fundamental to RNAi trigger molecule methodology, many modifications work at cross purposes with each other to optimize the behavior of the RNAi trigger. It is this balancing act which must be taken into account relative to the development of superior and effective RNAi molecules.

Another key factor is the stereochemical effect that arises in oligomers having P-chiral centers. In general, an oligomer with a length of n nucleosides will constitute a mixture of chirality in successive non-stereospecific chain synthesis. It has been observed that Rp and Sp homochiral chains, whose absolute configuration at all internucleotide methane phosphonate phosphorus atoms are either Rp or Sp, and non-stereoregular chains show different physicochemical properties as well as different capabilities of forming adducts with oligonucleotides of complementary sequence. In addition, phosphorothioate analogs of nucleotides have shown substantial stereoselectivity differences between Oligo-Rp and Oligo-Sp oligonucleotides in resistance to nucleases activity (Potter, BIOCHEMISTRY, 22:1369, (1983); Bryant et al., BIOCHEMISTRY, 18:2825, (1979)). Lesnikowski (NUCL. ACIDS RES., 18:2109, (1990)) observed that diastereomerically pure octathymidine methylphosphonates, in which six out of seven methylphosphonate bonds have defined configuration at the phosphorus atom when complexed with the matrix showed substantial differences in melting temperatures. According to the current disclosure chirally pure nucleotide analogs, or portions thereof, are expected to provide trigger structures with improved characteristics allowing the development of more potent and longer lasting RNAi triggers.

In some embodiments of the current disclosure it is particularly preferred to enhance stability, to include particular bases in overhangs, or to include modified nucleotides or nucleotide surrogates, in single strand overhangs, e.g., in a 5′ or 3′ overhang, or in both. Likewise, it can be desirable to include purine nucleotides in overhangs as they are more resistant to nuclease activity. In some embodiments all or some of the bases in a 3′ or 5′ overhang will be modified, with a modification described herein. Modifications can include the use of modifications at the 2′ OH group of the ribose sugar, deoxythymidine, instead of ribonucleotides, and modifications in the phosphate group, that is, phosphothioate modifications. Overhangs need not be homologous with the target sequence.

### Methods of Use

### Reducing Target Gene Expression

In some embodiments, the disclosure provides methods for contacting or delivering to a cell or population of cells an effective amount of any of the lipid-conjugated RNAi oligonucleotides herein to reduce expression of a target gene.

In some embodiments, expression of a target gene is reduced in one or more tissues or cells in a subject. In some embodiments, expression of a target gene is reduced in the central nervous system (CNS). In some embodiments, expression of a target gene is reduced in ocular tissue. In some embodiments, expression of a target gene is reduced in the liver. In some embodiments, expression of a target gene is reduced in adipose tissue. In some embodiments, expression of a target gene is reduced in adrenal tissue. In some embodiments, expression of a target gene is reduced in skeletal muscle tissue. In some embodiments, expression of a target gene is reduced in the heart. In some embodiments, expression of a target gene is reduced in the lung.

In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art.

Methods provided herein are useful in any appropriate cell type. In some embodiments, a cell is any cell that expresses the target mRNA. In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the lipid-conjugated RNAi oligonucleotides disclosed herein are delivered to a cell or population of cells using a nucleic acid delivery method known in the art including, but not limited to, injection of a solution or pharmaceutical composition containing the lipid-conjugated RNAi oligonucleotide, bombardment by particles covered by the lipid-conjugated RNAi oligonucleotide, exposing the cell or population of cells to a solution containing the lipid-conjugated RNAi oligonucleotide, or electroporation of cell membranes in the presence of the lipid-conjugated RNAi oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which a lipid-conjugated RNAi oligonucleotide provided herein reduces target gene expression in a cell is evaluated by comparing target gene expression in a cell or population of cells contacted with the lipid-conjugated RNAi oligonucleotide to a control cell or population of cells (e.g., a cell or population of cells not contacted with the lipid-conjugated RNAi oligonucleotide or contacted with a control lipid-conjugated RNAi oligonucleotide). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering a lipid-conjugated RNAi oligonucleotide described herein to a cell or a population of cells results in a reduction in expression of a target gene. In some embodiments, the reduction in target gene expression is relative to a control amount or level of target gene expression in cell or population of cells not contacted with the lipid-conjugated RNAi oligonucleotide or contacted with a control lipid-conjugated RNAi oligonucleotide. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with a lipid-conjugated RNAi oligonucleotide herein. In some embodiments, the effect of delivery of a lipid-conjugated RNAi oligonucleotide to a cell or population of cells according to a method herein is assessed after any finite period or amount of time (e.g., minutes, hours, days, weeks, months). For example, in some embodiments, target gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the lipid-conjugated RNAi oligonucleotide to the cell or population of cells. In some embodiments, target gene expression is determined in a cell or population of cells at least about 1 month, about 2 months, about 3 months, about 4 months, about 5 months, or about 6 months or more after contacting or delivering the lipid-conjugated RNAi oligonucleotide to the cell or population of cells.

### Reducing Target Gene Expression in the CNS

In some embodiments, expression of a target gene (e.g., neuronal target gene) is reduced in a region of the CNS. In some embodiments, expression of a target gene is reduced in at least one region of the CNS. In some embodiments, a region of the CNS is one or more tissues of the CNS. In some embodiments, regions of the CNS include, but are not limited to, cerebrum, prefrontal cortex, frontal cortex, motor cortex, temporal cortex, parietal cortex, occipital cortex, somatosensory cortex, hippocampus, caudate, striatum, globus pallidus, thalamus, midbrain, tegmentum, substantia nigra, pons, brainstem, cerebellar white matter, cerebellum, dentate nucleus, medulla, cervical spinal cord, thoracic spinal cord, lumbar spinal cord, cervical dorsal root ganglion, thoracic dorsal root ganglion, lumbar dorsal root ganglion, sacral dorsal root ganglion, nodose ganglia, femoral nerve, sciatic nerve, sural nerve, amygdala, hypothalamus, putamen, corpus callosum, and cranial nerve. In some embodiments, the region of the CNS is selected from the spinal cord, lumbar dorsal root ganglion, medulla, hippocampus, frontal cortex, brain stem, cerebellum, and a combination thereof. In some embodiments, expression of a target gene is reduced in at least one region of the CNS, selected from frontal cortex, medulla, hippocampus, hypothalamus, cerebellum, lumbar spinal cord, lumbar dorsal root ganglion, and any combination thereof.

In some embodiments, expression of a neuronal target gene is reduced in at least one tissue of the CNS. In some embodiments, expression of an astrocyte target gene is reduced in at least one tissue of the CNS. In some embodiments, expression of an oligodendrocyte target gene is reduced in at least one tissue of the CNS. In some embodiments, expression of a target mRNA in a neuron is reduced in at least one tissue of the CNS. In some embodiments, expression of a target mRNA in an astrocyte is reduced in at least one tissue of the CNS. In some embodiments, expression of a target mRNA in an oligodendrocyte is reduced in at least one tissue of the CNS.

In some embodiments, expression of a target mRNA in a neuron is reduced in at least one tissue of the CNS, selected from frontal cortex, medulla, hippocampus, hypothalamus, cerebellum, lumbar spinal cord, lumbar dorsal root ganglion, and any combination thereof. In some embodiments, expression of a target mRNA in an astrocyte is reduced in at least one tissue of the CNS, selected from frontal cortex, medulla, hippocampus, hypothalamus, cerebellum, lumbar spinal cord, lumbar dorsal root ganglion, and any combination thereof. In some embodiments, expression of a target mRNA in an oligodendrocyte is reduced in at least one tissue of the CNS, selected from frontal cortex, medulla, hippocampus, hypothalamus, cerebellum, lumbar spinal cord, lumbar dorsal root ganglion, and any combination thereof.

In some embodiments, expression of a target gene in the CNS of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a control tissue.

In some embodiments, expression of a target gene in an astrocyte of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a non-target cell.

In some embodiments, expression of a target gene in an oligodendrocyte of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a non-target cell.

In some embodiments, expression of a target gene in the neurons of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a non-target cell.

In some embodiments, contacting or delivering a lipid-conjugated RNAi oligonucleotide described herein to a cell or a population of cells results in a reduction in expression of a target gene in a neuron. In some embodiments, the reduction in expression of a target gene in a neuron is relative to an amount or level of target gene expression in an astrocyte contacted with the lipid-conjugated RNAi oligonucleotide. In some embodiments, the reduction expression of a target gene in a neuron is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of target gene expression in an astrocyte. In some embodiments, the reduction in expression of a target gene in a neuron is relative to an amount or level of target gene expression in an oligodendrocyte contacted with the lipid-conjugated RNAi oligonucleotide. In some embodiments, the reduction in expression of a target gene in a neuron is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of target gene expression in an oligodendrocyte.

In some embodiments, reduction in expression of a target gene in an astrocyte is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a neuron. In some embodiments, the reduction in expression of a target gene in an oligodendrocyte is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in a neuron.

In some embodiments, contacting or delivering a lipid-conjugated RNAi oligonucleotide described herein to a cell or a population of cells results in a reduction in expression of target gene in an astrocyte. In some embodiments, the reduction in expression of a target gene in an astrocyte is relative to an amount or level of target gene expression in a neuron contacted with the lipid-conjugated RNAi oligonucleotide. In some embodiments, the reduction in expression of a target gene in an astrocyte is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of expression of a target gene in a neuron. In some embodiments, the reduction in expression of a target gene in an astrocyte is relative to an amount or level of target gene expression in an oligodendrocytes contacted with the lipid-conjugated RNAi oligonucleotide. In some embodiments, the reduction in expression of a target gene in an astrocyte is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of target gene expression in an oligodendrocyte.

In some embodiments, reduction in expression of a target gene in a neuron is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in an astrocyte. In some embodiments, the reduction in expression of a target gene in an oligodendrocyte is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in an astrocyte.

In some embodiments, contacting or delivering a lipid-conjugated RNAi oligonucleotide described herein to a cell or a population of cells results in a reduction in expression of a target gene in an oligodendrocyte. In some embodiments, the reduction in oligodendrocyte target gene expression is relative to an amount or level of target gene expression in neurons contacted with the lipid-conjugated RNAi oligonucleotide. In some embodiments, the reduction in a target gene in an oligodendrocyte is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of target gene expression in a neuron. In some embodiments, the reduction in expression of a target gene in an oligodendrocyte is relative to an amount or level of expression of a target gene in an astrocyte contacted with the lipid-conjugated RNAi oligonucleotide. In some embodiments, the reduction in expression of a target gene in an oligodendrocyte is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a level of expression of a target gene in an astrocyte.

In some embodiments, reduction in expression of a target gene in a neuron is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in an oligodendrocyte. In some embodiments, the reduction in expression of a target gene in an astrocyte is increased by at least 1%, at least 5%, at least 10%, at least 15%, at least 20%, at least 25%, at least 30%, at least 35%, at least 40%, at least 45%, at least 50%, at least 55%, at least 60%, at least 70%, at least 80%, or at least 90% relative to reduction in expression of the target gene in an oligodendrocyte.

In some embodiments, contacting or delivering an oligonucleotide described herein to a cell or a population of cells results in a reduction in expression of a target gene in the CNS.

In some embodiments, differences in target mRNA expression between cell types or tissue types is measured using methods known in the art. In some embodiments, differences in target mRNA expression between cell types or tissue types measures the reduction of the target mRNA in a first cell/tissue type compared to the reduction of target mRNA in a second cell/tissue type.

For example, differences in target mRNA expression between cell types or tissue types is measured using polymerase chain reaction methods (e.g., RT-PCR) comparing relative expression between different tissue or cell types. In some embodiments, differences in target mRNA expression between cell types or tissue types is measured using Northern blot analysis, in situ hybridization, RT-PCR, RNA sequencing, or other methods known in the art. In some embodiments, a relative amount of target mRNA expression is compared between cell or tissue types. In some embodiments, an absolute amount of target mRNA expression is compared between cell or tissue types.

### Reducing Target Gene Expression in the Eye

In some embodiments, expression of a target gene is reduced in a region of the eye. In some embodiments, a region of the eye includes, but is not limited to, the optic nerve and retina.

In some embodiments, expression of a target gene in the ocular tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other control tissue.

### Reducing Target Gene Expression in the Liver

In some embodiments, expression of a target gene is reduced in a cell of the liver. In some embodiments, a cell of the liver is a macrophage located in the liver. In some embodiments, expression of a target gene is reduced in a region of the liver.

In some embodiments, expression of a target gene in the liver of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other tissue.

### Reducing Target Gene Expression in Muscle Tissue

In some embodiments, expression of a target gene is reduced in a cell of muscle tissue (e.g., skeletal muscle). In some embodiments, expression of a target gene in a muscle tissue (e.g., skeletal muscle) of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other tissue.

### Reducing Target Gene Expression in Adipose Tissue

In some embodiments, expression of a target gene is reduced in a cell of adipose tissue. In some embodiments, expression of a target gene in adipose tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other tissue.

### Reducing Target Gene Expression in Cardiac Tissue

In some embodiments, expression of a target gene is reduced in a cell of cardiac tissue. In some embodiments, expression of a target gene is reduced in a region of the heart. In some embodiments, expression of a target gene in cardiac tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other tissue.

### Reducing Target Gene Expression in Lung Tissue

In some embodiments, expression of a target gene is reduced in a cell of lung tissue. In some embodiments, expression of a target gene is reduced in a region of a lung. In some embodiments, expression of a target gene in cardiac tissue of a subject is reduced by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in other tissue.

### Treatment Methods

In some embodiments, the disclosure provides methods for treating a disease, disorder, or condition associated with expression of a target gene in one or more tissues or cells. In some embodiments, the disclosure provides methods for treating a disease, disorder, or condition associated with expression of a target gene in the CNS (e.g., neuronal gene). In some embodiments, the disclosure provides methods for treating a disease, disorder, or condition associated with expression of a target gene in ocular tissue. In some embodiments, the disclosure provides methods for treating a disease, disorder, or condition associated with expression of a target gene in the liver (e.g., macrophage target gene). In some embodiments, the disclosure provides methods for treating a disease, disorder, or condition associated with expression of a target gene in adipose tissue. In some embodiments, the disclosure provides methods for treating a disease, disorder, or condition associated with expression of a target gene in adrenal tissue. In some embodiments, the disclosure provides methods for treating a disease, disorder, or condition associated with expression of a target gene in skeletal muscle tissue. In some embodiments, the disclosure provides methods for treating a disease, disorder, or condition associated with expression of a target gene in lung tissue. In some embodiments, the disclosure provides methods for treating a disease, disorder, or condition associated with expression of a target gene in heart tissue.

Methods described herein are typically involve administering to a subject a therapeutically effective amount of a lipid-conjugated RNAi oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the brain of a subject).

In some embodiments, a lipid-conjugated RNAi oligonucleotide herein, or a composition thereof, is administered once every year, once every 6 months, once every 4 months, quarterly (once every three months), bi-monthly (once every two months), monthly or weekly. In some embodiments, a lipid-conjugated RNAi oligonucleotide herein, or a composition thereof, is administered every week or at intervals of two, or three weeks. In some embodiments, a lipid-conjugated RNAi oligonucleotide herein, or a composition thereof, is administered daily. In some embodiments, a subject is administered one or more loading doses of a lipid-conjugated RNAi oligonucleotide herein, or a composition thereof, followed by one or more maintenance doses of the lipid-conjugated RNAi oligonucleotide, or a composition thereof.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Treatment Methods in the CNS

The disclosure provides oligonucleotides for use as a medicament, in particular for use in a method for the treatment of diseases, disorders, and conditions associated with the CNS. The disclosure also provides lipid-conjugated RNAi oligonucleotide s for use, or adaptable for use, to treat a subject (e.g., a human) having a disease, disorder or condition associated with expression of a target gene (e.g., neuronal gene) that would benefit from reducing expression of the target gene. In some embodiments, the disclosure provides lipid-conjugated RNAi oligonucleotides for use, or adapted for use, to treat a subject having a disease, disorder or condition associated with expression of a target gene in the CNS. The disclosure also provides lipid-conjugated RNAi oligonucleotides for use, or adaptable for use, in the manufacture of a medicament or pharmaceutical composition for treating a disease, disorder or condition associated with expression of a target gene in the CNS. In some embodiments, the lipid-conjugated RNAi oligonucleotides for use, or adaptable for use, target mRNA and reduce expression of a target gene in the CNS (e.g., via the RNAi pathway). In some embodiments, the lipid-conjugated RNAi oligonucleotides for use, or adaptable for use, target mRNA and reduce the amount or level of target mRNA, protein and/or activity.

In addition, in some embodiments of the methods herein, a subject having a disease, disorder or condition associated with expression of a target gene in the CNS or is predisposed to the same is selected for treatment with a lipid-conjugated RNAi oligonucleotide herein. In some embodiments, the method comprises selecting an individual having a marker (e.g., a biomarker) for a disease, disorder or condition associated with expression of a target gene in the CNS, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a target gene in the CNS, and then comparing such obtained value to one or more other baseline values or values obtained after the subject is administered the lipid-conjugated RNAi oligonucleotide to assess the effectiveness of treatment.

The disclosure also provides methods of treating a subject having, suspected of having, or at risk of developing a disease, disorder or condition associated with expression of a target gene in the CNS e with a lipid-conjugated RNAi oligonucleotide provided herein. In some embodiments, the disclosure provides methods of treating or attenuating the onset or progression of a disease, disorder or condition associated with expression of a target gene in the CNS using the lipid-conjugated RNAi oligonucleotides provided herein. In some embodiments, the disclosure provides methods to achieve one or more therapeutic benefits in a subject having a disease, disorder or condition associated with expression of a target gene in the CNS using the lipid-conjugated RNAi oligonucleotides provided herein.

In some embodiments of the methods herein, the subject is treated by administering a therapeutically effective amount of any one or more of the lipid-conjugated RNAi oligonucleotides provided herein. In some embodiments, treatment comprises reducing expression of a target gene in the CNS (e.g., neuronal target gene). In some embodiments, the subject is treated therapeutically. In some embodiments, the subject is treated prophylactically.

In some embodiments of the methods herein, a lipid-conjugated RNAi oligonucleotide provided herein, or a pharmaceutical composition comprising the lipid-conjugated RNAi oligonucleotide, is administered to a subject having a disease, disorder or condition associated with expression of a target gene in the CNS (e.g., neuronal target gene) such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, a lipid-conjugated RNAi oligonucleotide provided herein, or a pharmaceutical composition comprising the lipid-conjugated RNAi oligonucleotide, is administered to a subject having a disease, disorder or condition associated with expression of a target gene in the CNS (e.g., neuronal target gene) such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the lipid-conjugated RNAi oligonucleotide or pharmaceutical composition. In some embodiments, expression of a target gene in the CNS (e.g., neuronal target gene) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, an lipid-conjugated RNAi oligonucleotide herein, or a pharmaceutical composition comprising the lipid-conjugated RNAi oligonucleotide, is administered to a subject having a disease, disorder or condition associated with expression of a target gene in the CNS (e.g., neuronal target gene) such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the lipid-conjugated RNAi oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, a lipid-conjugated RNAi oligonucleotide herein, or a pharmaceutical composition comprising the lipid-conjugated RNAi oligonucleotide, is administered to a subject having a disease, disorder or condition associated with expression of a target gene in the CNS (e.g., neuronal target gene) such that an amount or level of protein encoded by the target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the lipid-conjugated RNAi oligonucleotide or pharmaceutical composition. In some embodiments, an amount or level of protein encoded by a target gene in the CNS (e.g., neuronal target gene) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

In some embodiments of the methods herein, a lipid-conjugated RNAi oligonucleotide herein, or a pharmaceutical composition comprising the lipid-conjugated RNAi oligonucleotide, is administered to a subject having a disease, disorder or condition associated with expression of a target gene in the CNS (e.g., neuronal target gene) such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the lipid-conjugated RNAi oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the lipid-conjugated RNAi oligonucleotide or pharmaceutical composition or receiving a control lipid-conjugated RNAi oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell (e.g., a neuron), a population or a group of cells (e.g., an organoid), an organ (e.g., CNS), blood or a fraction thereof (e.g., plasma), a tissue (e.g., brain tissue), a sample (e.g., CSF sample or a brain biopsy sample), or any other biological material obtained or isolated from the subject. In some embodiments, expression of a target gene in the CNS (e.g., neuronal target gene), an amount or level of target gene mRNA, an amount or level of protein encoded by the target gene, an amount or level of target gene activity, or any combination thereof, is reduced in more than one type of cell (e.g., neuron), more than one groups of cells, more than one organ (e.g., brain and one or more other organ(s)), more than one fraction of blood (e.g., plasma and one or more other blood fraction(s)), more than one type of tissue (e.g., brain tissue and one or more other type(s) of tissue), more than one type of sample (e.g., a brain biopsy sample and one or more other type(s) of biopsy sample) obtained or isolated from the subject.

In some embodiments, expression of a target mRNA is reduced in one or more of astrocytes, neurons, or oligodendrocytes. In some embodiments, expression of a target mRNA is reduced in astrocytes. In some embodiments, expression of a target mRNA is reduced in neurons. In some embodiments, expression of a target mRNA is reduced in oligodendrocytes. In some embodiments, expression of a target mRNA is reduced in astrocytes and neurons. In some embodiments, expression of a target mRNA is reduced in astrocytes and oligodendrocytes. In some embodiments, expression of a target mRNA is reduced in neurons and oligodendrocytes. In some embodiments, expression of a target mRNA is reduced in astrocytes, oligodendrocytes, and neurons.

In some embodiments, expression of a target gene in the CNS (e.g., neuronal target gene) is reduced in one or more of the spinal cord, lumbar dorsal root ganglion (DRG), medulla, hippocampus, frontal cortex, brainstem, or cerebellum. In some embodiments, expression of a target gene in the CNS (e.g., neuronal target gene) is reduced in one or more of the spinal cord, lumbar spinal cord, thoracic spinal cord, cervical spinal cord, lumbar dorsal root ganglion (DRG), medulla, hippocampus, frontal cortex, brainstem, hypothalamus, or cerebellum. In some embodiments, expression of a target gene in the CNS (e.g., neuronal target gene) is reduced in the spinal cord. In some embodiments, expression of a target gene in the CNS (e.g., neuronal target gene) is reduced in the lumbar spinal cord. In some embodiments, expression of a target gene in the CNS (e.g., neuronal target gene) is reduced in the thoracic spinal cord. In some embodiments, expression of a target gene in the CNS (e.g., neuronal target gene) is reduced in the cervical spinal cord. In some embodiments, expression of a target gene in the CNS (e.g., neuronal target gene) is reduced in lumbar dorsal root ganglion. In some embodiments, expression of a target gene in the CNS (e.g., neuronal target gene) is reduced in the medulla. In some embodiments, expression of a target gene in the CNS (e.g., neuronal target gene) is reduced in the hippocampus. In some embodiments, expression of a target gene in the CNS (e.g., neuronal target gene) is reduced in the frontal cortex. In some embodiments, expression of a target gene in the CNS (e.g., neuronal target gene) is reduced in the brainstem. In some embodiments, expression of a target gene in the CNS (e.g., neuronal target gene) is reduced in the hypothalamus. In some embodiments, expression of a target gene in the CNS (e.g., neuronal target gene) is reduced in the cerebellum.

In some embodiments, the disclosure provides a method of reducing expression of a target mRNA in an astrocyte, comprising administering a blunt-end double-stranded oligonucleotide wherein the oligonucleotide comprises:
(i) a sense strand, wherein the sense strand is 20 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to the target mRNA in the astrocyte, and
wherein the sense and antisense strand are separate strands which form an asymmetric duplex region having an overhang of 2 nucleotides at the 3′ terminus of the antisense strand, wherein the duplex region is 20 nucleotides, and
wherein the oligonucleotide does not reduce the target mRNA in a neuron to the same amount as reduction in the astrocyte.

In some embodiments, the disclosure provides a method of reducing expression of a target mRNA in an astrocyte, comprising administering a double-stranded oligonucleotide wherein the oligonucleotide comprises:
(i) a sense strand, wherein the sense strand is 14 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to the target mRNA in the astrocyte, and
wherein the sense and antisense strand are separate strands which form an asymmetric duplex region having an overhang of 8 nucleotides at the 3′ terminus of the antisense strand, wherein the duplex region is 14 nucleotides,
wherein the oligonucleotide does not reduce target mRNA in an oligodendrocyte to the same or similar amount as reduction in the astrocyte.

In some embodiments, the disclosure provides a method of reducing expression of a target mRNA in an astrocyte and an oligodendrocyte to the same or similar amount, comprising administering a double-stranded oligonucleotide wherein the oligonucleotide comprises:
(i) a sense strand, wherein the sense strand is 20 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to the target mRNA in the astrocyte and oligodendrocyte, and
wherein the sense and antisense strand are separate strands which form an asymmetric duplex region having an overhang of 2 nucleotides at the 3′ terminus of the antisense strand, wherein the duplex region is 20 nucleotides, thereby reducing expression of the target mRNA in the astrocyte and oligodendrocyte to the same or similar amount.

In some embodiments, the disclosure provides a method of reducing expression of a target mRNA in an astrocyte and a neuron, comprising administering a double-stranded oligonucleotide wherein the oligonucleotide comprises:
(i) a sense strand, wherein the sense strand is 14 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to the target mRNA in the astrocyte and neuron, and
wherein the sense and antisense strand are separate strands which form an asymmetric duplex region having an overhang of 8 nucleotides at the 3′ terminus of the antisense strand, wherein the duplex region is 14 nucleotides,
wherein the oligonucleotide does not reduce the target mRNA in an oligodendrocyte to the same amount as reducing in the astrocyte and neuron.

In some embodiments, the disclosure provides a method of reducing expression of a target mRNA in a neuron, comprising administering a double-stranded oligonucleotide wherein the oligonucleotide comprises:
(i) a sense strand, wherein the sense strand is 14 nucleotides in length, wherein the sense strand comprises at least one lipid moiety conjugated to the 5′ terminus, and wherein the sense strand comprises at least one locked nucleic acid but no more than 5 locked nucleic acids, and
(ii) an antisense strand, wherein the antisense strand is 22 nucleotides in length, and wherein the antisense strand comprises a region of complementarity to the target mRNA in the neuron, and
wherein the sense and antisense strand are separate strands which form an asymmetric duplex region having an overhang of about 8 nucleotides at the 3′ terminus of the antisense strand, wherein the duplex region is about 14 nucleotides.

In some embodiments, the disclosure provides a method of reducing expression of a target mRNA in a neuron, comprising administering a double-stranded oligonucleotide wherein the oligonucleotide comprises:
(i) a sense strand, wherein the sense strand is 14 nucleotides in length, wherein the sense strand comprises at least one lipid moiety conjugated to the 5′ terminus, and wherein the sense strand comprises at least one locked nucleic acid, and
(ii) an antisense strand, wherein the antisense strand is 22 nucleotides in length, and wherein the antisense strand comprises a region of complementarity to the target mRNA in the neuron, and
wherein the sense and antisense strand are separate strands which form an asymmetric duplex region having an overhang of about 8 nucleotides at the 3′ terminus of the antisense strand, wherein the duplex region is about 14 nucleotides, wherein the oligonucleotide does not reduce the target mRNA in an oligodendrocyte to the same amount as reducing in the neuron.

In some embodiments, the disclosure provides a method of reducing expression of a target mRNA in a neuron in the lumbar dorsal root ganglion (DRG), comprising administering a double-stranded oligonucleotide wherein the oligonucleotide comprises:
(i) a sense strand, wherein the sense strand is 26 nucleotides in length, wherein the sense strand comprises a stem-loop comprising a lipid moiety conjugated to the stem-loop, and wherein the sense strand comprises a locked nucleic acid at the 5′ terminus, and
(ii) an antisense strand, wherein the antisense strand is 22 nucleotides in length, wherein the antisense strand comprises a phosphorothioate linkage between position 1 and 2, position 2 and 3, position 3 and 4, position 12 and 13, position 13 and 14, position 14 and 15, position 15 and 16, position 16 and 17, position 17 and 18, position 18 and 19, position 19 and 20, position 20 and 21, and position 21 and 22, and wherein the antisense strand comprises a region of complementarity to the target mRNA in the neuron, and
wherein the sense and antisense strand are separate strands which form an asymmetric duplex region having an overhang of about 8 nucleotides at the 3′ terminus of the antisense strand, wherein the duplex region is about 14 nucleotides.

Examples of a disease, disorder or condition associated with expression of a target gene in the CNS (e.g., neuronal target gene), but are not limited to, Pelizacus-Merzbacher Disease, Spinal Cord Injury & Stroke, Krabbe Disease, Metachromatic Leukodystrophy, Adult-Onset Leukodystrophy, Multiple Sclerosis, X-linked Adrenoleukodystrophy, Progressive Supranuclear Palsy (PSP), Corticobasal degeneration (CBD), Argyrophilic grain disease (AGD), Globular glial tauopathy (GGT), Ageing-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MDI or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 10), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), spinal cord injury, Dentatorubral-pallidoluysian atrophy (DRPLA), recessive CNS disorders, ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizacus-Merzbacher disease (PMD), Lafora disease, stroke, Cerebral Amyloid Angiopathy (CAA), and Metachromatic Leukodystrophy (MLD).

In some embodiments, the target gene in the CNS (e.g., neuronal target gene) may be a target gene from any mammal, such as a human. Any target gene in the CNS (e.g., neuronal target gene) may be silenced according to the method described herein.

In some embodiments, a lipid-conjugated RNAi oligonucleotide herein, or a composition thereof, is administered intrathecally into cerebrospinal fluid (CSF) (e.g., injection or infusion into the fluid within the subarachnoid space). In some embodiments, intrathecal administration of a lipid-conjugated RNAi oligonucleotide herein, or a composition thereof, is performed as a bolus injection into the subarachnoid space. In some embodiments, intrathecal administration of a lipid-conjugated RNAi oligonucleotide herein, or a composition thereof, is performed as an infusion into the subarachnoid space. In some embodiments, intrathecal administration of a herein, or a composition thereof, is performed via a catheter into the subarachnoid space. In some embodiments, intrathecal administration of a lipid-conjugated RNAi oligonucleotide herein, or a composition thereof, is performed via a pump. In some embodiments, intrathecal administration of a lipid-conjugated RNAi oligonucleotide herein, or a composition thereof, is performed via an implantable pump. In some embodiments, administration is performed via an implantable device that operates or functions a reservoir.

In some embodiments, a lipid-conjugated RNAi oligonucleotide herein, or a composition thereof, is administered intrathecally into the cerebellomedullary cistern (also referred to as the cisterna magna). Intrathecal administration into the cisterna magna is referred to as “intracisternal administration” or “intracisternal magna (i.c.m.) administration). In some embodiments, a lipid-conjugated RNAi oligonucleotide herein, or composition thereof, is administered intrathecally into the subarachnoid space of the lumbar spinal cord. Intrathecal administration into the subarachnoid space of the lumbar spinal cord is referred to as “lumbar intrathecal (i.t.) administration”. In some embodiments, a lipid-conjugated RNAi oligonucleotide herein, or composition thereof, is administered intrathecally into the subarachnoid space of the cervical spinal cord. Intrathecal administration into the subarachnoid space of the cervical spinal cord is referred to as “cervical intrathecal (i.t.) administration”. In some embodiments, a lipid-conjugated RNAi oligonucleotide herein, or composition thereof, is administered intrathecally into the subarachnoid space of the thoracic spinal cord. Intrathecal administration into the subarachnoid space of the thoracic spinal cord is referred to as “thoracic intrathecal (i.t.) administration”. In some embodiments, a lipid-conjugated RNAi oligonucleotide herein, or composition thereof, is administered by intracerebroventricular injection or infusion into the cerebral ventricles. Intracerebroventricular administration into the ventricular space is referred to as “intracerebroventricular (i.c.v.) administration”. In some embodiments, an Ommaya reservoir is used to administer a lipid-conjugated RNAi oligonucleotide herein, or composition thereof, by intracerebroventricular injection or infusion.

In some embodiments, a lipid-conjugated RNAi oligonucleotide herein, or a composition thereof, is administered via ophthalmic, intraocular, subconjunctival, intravitreal, retrobulbar, or intracameral administration.

In some embodiments, a lipid-conjugated RNAi oligonucleotide herein, or a composition thereof, is administered via epidural administration.

### Treatment Methods in Ocular Tissue

The disclosure provides oligonucleotides for use as a medicament, in particular for use in a method for the treatment of diseases, disorders, and conditions associated with ocular tissue. The disclosure also provides oligonucleotide s for use, or adaptable for use, to treat a subject (e.g., a human) having a disease, disorder or condition associated with expression of an ocular target gene that would benefit from reducing expression of the ocular target gene. In some embodiments, the disclosure provides oligonucleotides for use, or adapted for use, to treat a subject having a disease, disorder or condition associated with ocular target gene expression. The disclosure also provides oligonucleotides for use, or adaptable for use, in the manufacture of a medicament or pharmaceutical composition for treating a disease, disorder or condition associated with expression of an ocular target gene. In some embodiments, the oligonucleotides for use, or adaptable for use, target mRNA and reduce expression of an ocular target gene (e.g., via the RNAi pathway). In some embodiments, the oligonucleotides for use, or adaptable for use, target mRNA and reduce the amount or level of ocular target mRNA, protein and/or activity.

In addition, in some embodiments of the methods herein, a subject having a disease, disorder or condition associated with expression of an ocular target gene or is predisposed to the same is selected for treatment with an oligonucleotide herein. In some embodiments, the method comprises selecting an individual having a marker (e.g., a biomarker) for a disease, disorder or condition associated with expression of an ocular target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of an ocular target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of an ocular target gene with an oligonucleotide provided herein. In some embodiments, the disclosure provides methods of treating or attenuating the onset or progression of a disease, disorder or condition associated with expression of an ocular target gene using the oligonucleotides provided herein. In some embodiments, the disclosure provides methods to achieve one or more therapeutic benefits in a subject having a disease, disorder or condition associated with expression of an ocular target gene using the oligonucleotides provided herein.

In some embodiments of the methods herein, the subject is treated by administering a therapeutically effective amount of any one or more of the oligonucleotides provided herein. In some embodiments, treatment comprises reducing expression of an ocular target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of an ocular target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of an ocular target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of an ocular target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an ocular target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an ocular target gene such that an amount or level of protein encoded by the ocular target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by an ocular target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an ocular target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell (e.g., a retinal cell), a population or a group of cells (e.g., an organoid), an organ (e.g., ocular), blood or a fraction thereof (e.g., plasma), a tissue (e.g., ocular tissue), a sample (e.g., an ocular biopsy sample), or any other biological material obtained or isolated from the subject. In some embodiments, expression of an ocular target gene, an amount or level of target gene mRNA, an amount or level of protein encoded by the target gene, an amount or level of target gene activity, or any combination thereof, is reduced in more than one type of cell (e.g., a retinal cell), more than one groups of cells, more than one organ (e.g., ocular and one or more other organ(s)), more than one fraction of blood (e.g., plasma and one or more other blood fraction(s)), more than one type of tissue (e.g., ocular tissue and one or more other type(s) of tissue), more than one type of sample (e.g., an ocular biopsy sample and one or more other type(s) of biopsy sample) obtained or isolated from the subject.

In some embodiments, the ocular target gene may be a target gene from any mammal, such as a human. Any ocular gene may be silenced according to the method described herein.

In some embodiments, an oligonucleotide herein, or a composition thereof, is administered via ophthalmic, intraocular, subconjunctival, intravitreal, retrobulbar, or intracameral administration.

### Treatment Methods in the Liver

The disclosure provides oligonucleotides for use as a medicament, in particular for use in a method for the treatment of diseases, disorders, and conditions associated with the liver. The disclosure also provides oligonucleotides for use, or adaptable for use, to treat a subject (e.g., a human) having a disease, disorder or condition associated with expression of a liver target gene (e.g., liver macrophage target gene) that would benefit from reducing expression of the liver target gene. In some embodiments, the disclosure provides oligonucleotides for use, or adapted for use, to treat a subject having a disease, disorder or condition associated with liver target gene (e.g., liver macrophage target gene) expression. The disclosure also provides oligonucleotides for use, or adaptable for use, in the manufacture of a medicament or pharmaceutical composition for treating a disease, disorder or condition associated with expression of a liver target gene (e.g., liver macrophage target gene). In some embodiments, the oligonucleotides for use, or adaptable for use, target mRNA and reduce expression of a liver target gene (e.g., via the RNAi pathway). In some embodiments, the oligonucleotides for use, or adaptable for use, target mRNA and reduce the amount or level of liver target mRNA, protein and/or activity.

In addition, in some embodiments of the methods herein, a subject having a disease, disorder or condition associated with expression of a liver target gene (e.g., liver macrophage target gene) or is predisposed to the same is selected for treatment with an oligonucleotide herein. In some embodiments, the method comprises selecting an individual having a marker (e.g., a biomarker) for a disease, disorder or condition associated with expression of a liver target gene (e.g., liver macrophage target gene), or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a liver target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of a liver target gene with an oligonucleotide provided herein. In some embodiments, the disclosure provides methods of treating or attenuating the onset or progression of a disease, disorder or condition associated with expression of a liver target gene (e.g., liver macrophage target gene) using the oligonucleotides provided herein. In some embodiments, the disclosure provides methods to achieve one or more therapeutic benefits in a subject having a disease, disorder or condition associated with expression of a liver target gene (e.g., liver macrophage target gene) using the oligonucleotides provided herein.

In some embodiments of the methods herein, the subject is treated by administering a therapeutically effective amount of any one or more of the oligonucleotides provided herein. In some embodiments, treatment comprises reducing expression of a liver target gene (e.g., liver macrophage target gene). In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of a liver target gene (e.g., liver macrophage target gene) such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of a liver target gene (e.g., liver macrophage target gene) such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of a liver target gene (e.g., liver macrophage target gene) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene (e.g., liver macrophage target gene) such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene (e.g., liver macrophage target gene) such that an amount or level of protein encoded by the liver target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by a liver target gene (e.g., liver macrophage target gene) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene (e.g., liver macrophage target gene) such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell (e.g., a liver macrophage), a population or a group of cells (e.g., an organoid), an organ (e.g., liver), blood or a fraction thereof (e.g., plasma), a tissue (e.g., liver tissue), a sample (e.g., liver biopsy sample), or any other biological material obtained or isolated from the subject. In some embodiments, expression of a liver target gene, an amount or level of target gene mRNA, an amount or level of protein encoded by the target gene, an amount or level of target gene activity, or any combination thereof, is reduced in more than one type of cell (e.g., a liver macrophage), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), more than one type of sample (e.g., a liver biopsy sample and one or more other type(s) of biopsy sample) obtained or isolated from the subject.

In some embodiments, the liver target gene may be a target gene from any mammal, such as a human. Any liver gene may be silenced according to the method described herein.

In some embodiments, an oligonucleotide herein, or a composition thereof, is administered via subcutaneous or intravenous administration.

### Treatment Methods in Adipose Tissue

The disclosure provides oligonucleotides for use as a medicament, in particular for use in a method for the treatment of diseases, disorders, and conditions associated with adipose tissue. The disclosure also provides oligonucleotides for use, or adaptable for use, to treat a subject (e.g., a human) having a disease, disorder or condition associated with expression of an adipose tissue target gene that would benefit from reducing expression of the adipose tissue target gene. In some embodiments, the disclosure provides oligonucleotides for use, or adapted for use, to treat a subject having a disease, disorder or condition associated with adipose tissue target gene expression. The disclosure also provides oligonucleotides for use, or adaptable for use, in the manufacture of a medicament or pharmaceutical composition for treating a disease, disorder or condition associated with expression of an adipose tissue target gene. In some embodiments, the oligonucleotides for use, or adaptable for use, target mRNA and reduce expression of an adipose tissue target gene (e.g., via the RNAi pathway). In some embodiments, the oligonucleotides for use, or adaptable for use, target mRNA and reduce the amount or level of adipose tissue target mRNA, protein and/or activity.

In addition, in some embodiments of the methods herein, a subject having a disease, disorder or condition associated with expression of an adipose tissue target gene or is predisposed to the same is selected for treatment with an oligonucleotide herein. In some embodiments, the method comprises selecting an individual having a marker (e.g., a biomarker) for a disease, disorder or condition associated with expression of an adipose tissue target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of an adipose tissue target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of an adipose tissue target gene with an oligonucleotide provided herein. In some embodiments, the disclosure provides methods of treating or attenuating the onset or progression of a disease, disorder or condition associated with expression of an adipose tissue target gene using the oligonucleotides provided herein. In some embodiments, the disclosure provides methods to achieve one or more therapeutic benefits in a subject having a disease, disorder or condition associated with expression of an adipose tissue target gene using the oligonucleotides provided herein.

In some embodiments of the methods herein, the subject is treated by administering a therapeutically effective amount of any one or more of the oligonucleotides provided herein. In some embodiments, treatment comprises reducing expression of an adipose tissue target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of an adipose tissue target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of an adipose tissue target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of an adipose tissue target is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adipose tissue target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adipose tissue target gene such that an amount or level of protein encoded by the adipose target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by an adipose tissue target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adipose tissue target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ, blood or a fraction thereof (e.g., plasma), a tissue (e.g., adipose tissue), a sample (e.g., adipose biopsy sample), or any other biological material obtained or isolated from the subject. In some embodiments, expression of an adipose tissue target gene, an amount or level of target gene mRNA, an amount or level of protein encoded by the target gene, an amount or level of target gene activity, or any combination thereof, is reduced in more than one type of cell, more than one groups of cells, more than one organ (e.g., adipose and one or more other organ(s)), more than one fraction of blood (e.g., plasma and one or more other blood fraction(s)), more than one type of tissue (e.g., adipose tissue and one or more other type(s) of tissue), more than one type of sample (e.g., a adipose biopsy sample and one or more other type(s) of biopsy sample) obtained or isolated from the subject.

In some embodiments, the adipose tissue target gene may be a target gene from any mammal, such as a human. Any adipose tissue gene may be silenced according to the method described herein.

In some embodiments, an oligonucleotide herein, or a composition thereof, is administered via subcutaneous or intravenous administration.

### Treatment Methods in Adrenal Tissue

The disclosure provides oligonucleotides for use as a medicament, in particular for use in a method for the treatment of diseases, disorders, and conditions associated with adrenal tissue. The disclosure also provides oligonucleotides for use, or adaptable for use, to treat a subject (e.g., a human) having a disease, disorder or condition associated with expression of an adrenal tissue target gene that would benefit from reducing expression of the adrenal tissue target gene. In some embodiments, the disclosure provides oligonucleotides for use, or adapted for use, to treat a subject having a disease, disorder or condition associated with adrenal tissue target gene expression. The disclosure also provides oligonucleotides for use, or adaptable for use, in the manufacture of a medicament or pharmaceutical composition for treating a disease, disorder or condition associated with expression of an adrenal tissue target gene. In some embodiments, the oligonucleotides for use, or adaptable for use, target mRNA and reduce expression of an adrenal tissue target gene (e.g., via the RNAi pathway). In some embodiments, the oligonucleotides for use, or adaptable for use, target mRNA and reduce the amount or level of adrenal tissue target mRNA, protein and/or activity.

In addition, in some embodiments of the methods herein, a subject having a disease, disorder or condition associated with expression of an adrenal tissue target gene or is predisposed to the same is selected for treatment with an oligonucleotide herein. In some embodiments, the method comprises selecting an individual having a marker (e.g., a biomarker) for a disease, disorder or condition associated with expression of an adrenal tissue target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of an adrenal tissue target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of an adrenal tissue target gene with an oligonucleotide provided herein. In some embodiments, the disclosure provides methods of treating or attenuating the onset or progression of a disease, disorder or condition associated with expression of an adrenal tissue target gene using the oligonucleotides provided herein. In some embodiments, the disclosure provides methods to achieve one or more therapeutic benefits in a subject having a disease, disorder or condition associated with expression of an adrenal tissue target gene using the oligonucleotides provided herein.

In some embodiments of the methods herein, the subject is treated by administering a therapeutically effective amount of any one or more of the oligonucleotides provided herein. In some embodiments, treatment comprises reducing expression of an adrenal tissue target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of an adrenal tissue target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of an adrenal tissue target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of an adrenal tissue target is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adrenal tissue target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adrenal tissue target gene such that an amount or level of protein encoded by the adrenal target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by an adrenal tissue target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of an adrenal tissue target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ, blood or a fraction thereof (e.g., plasma), a tissue (e.g., adrenal tissue), a sample (e.g., adrenal biopsy sample), or any other biological material obtained or isolated from the subject. In some embodiments, expression of an adrenal tissue target gene, an amount or level of target gene mRNA, an amount or level of protein encoded by the target gene, an amount or level of target gene activity, or any combination thereof, is reduced in more than one type of cell, more than one groups of cells, more than one organ (e.g., adrenal and one or more other organ(s)), more than one fraction of blood (e.g., plasma and one or more other blood fraction(s)), more than one type of tissue (e.g., adrenal tissue and one or more other type(s) of tissue), more than one type of sample (e.g., a adrenal biopsy sample and one or more other type(s) of biopsy sample) obtained or isolated from the subject.

In some embodiments, the adrenal tissue target gene may be a target gene from any mammal, such as a human. Any adrenal tissue gene may be silenced according to the method described herein.

In some embodiments, an oligonucleotide herein, or a composition thereof, is administered via subcutaneous or intravenous administration.

### Treatment Methods in Muscle Tissue

The disclosure provides oligonucleotides for use as a medicament, in particular for use in a method for the treatment of diseases, disorders, and conditions associated with muscle tissue. The disclosure also provides oligonucleotides for use, or adaptable for use, to treat a subject (e.g., a human) having a disease, disorder or condition associated with expression of a muscle target gene that would benefit from reducing expression of the muscle target gene. In some embodiments, the disclosure provides oligonucleotides for use, or adapted for use, to treat a subject having a disease, disorder or condition associated with muscle target gene expression. The disclosure also provides oligonucleotides for use, or adaptable for use, in the manufacture of a medicament or pharmaceutical composition for treating a disease, disorder or condition associated with expression of a muscle target gene. In some embodiments, the oligonucleotides for use, or adaptable for use, target mRNA and reduce expression of a muscle target gene (e.g., via the RNAi pathway). In some embodiments, the oligonucleotides for use, or adaptable for use, target mRNA and reduce the amount or level of muscle target mRNA, protein and/or activity.

In addition, in some embodiments of the methods herein, a subject having a disease, disorder or condition associated with expression of a muscle target gene or is predisposed to the same is selected for treatment with an oligonucleotide herein. In some embodiments, the method comprises selecting an individual having a marker (e.g., a biomarker) for a disease, disorder or condition associated with expression of a muscle target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a muscle target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of a muscle target gene with an oligonucleotide provided herein. In some embodiments, the disclosure provides methods of treating or attenuating the onset or progression of a disease, disorder or condition associated with expression of a muscle target gene using the oligonucleotides provided herein. In some embodiments, the disclosure provides methods to achieve one or more therapeutic benefits in a subject having a disease, disorder or condition associated with expression of a muscle target gene using the oligonucleotides provided herein.

In some embodiments of the methods herein, the subject is treated by administering a therapeutically effective amount of any one or more of the oligonucleotides provided herein. In some embodiments, treatment comprises reducing expression of a muscle target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of a muscle target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of a muscle target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of a muscle target is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a muscle target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a muscle target gene such that an amount or level of protein encoded by the muscle target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by a muscle target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a muscle target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ, blood or a fraction thereof (e.g., plasma), a tissue (e.g., muscle tissue), a sample (e.g., muscle biopsy sample), or any other biological material obtained or isolated from the subject. In some embodiments, expression of a muscle target gene, an amount or level of target gene mRNA, an amount or level of protein encoded by the target gene, an amount or level of target gene activity, or any combination thereof, is reduced in more than one type of cell, more than one groups of cells, more than one organ (e.g., muscle and one or more other organ(s)), more than one fraction of blood (e.g., plasma and one or more other blood fraction(s)), more than one type of tissue (e.g., muscle tissue and one or more other type(s) of tissue), more than one type of sample (e.g., muscle biopsy sample and one or more other type(s) of biopsy sample) obtained or isolated from the subject.

In some embodiments, the muscle target gene may be a target gene from any mammal, such as a human. Any muscle gene may be silenced according to the method described herein.

In some embodiments, an oligonucleotide herein, or a composition thereof, is administered via subcutaneous or intravenous administration.

### Treatment Methods in Heart Tissue

The disclosure provides oligonucleotides for use as a medicament, in particular for use in a method for the treatment of diseases, disorders, and conditions associated with heart tissue. The disclosure also provides oligonucleotides for use, or adaptable for use, to treat a subject (e.g., a human) having a disease, disorder or condition associated with expression of a heart target gene that would benefit from reducing expression of the heart target gene. In some embodiments, the disclosure provides oligonucleotides for use, or adapted for use, to treat a subject having a disease, disorder or condition associated with heart target gene expression. The disclosure also provides oligonucleotides for use, or adaptable for use, in the manufacture of a medicament or pharmaceutical composition for treating a disease, disorder or condition associated with expression of a heart target gene. In some embodiments, the oligonucleotides for use, or adaptable for use, target mRNA and reduce expression of a heart target gene (e.g., via the RNAi pathway). In some embodiments, the oligonucleotides for use, or adaptable for use, target mRNA and reduce the amount or level of heart target mRNA, protein and/or activity.

In addition, in some embodiments of the methods herein, a subject having a disease, disorder or condition associated with expression of a heart target gene or is predisposed to the same is selected for treatment with an oligonucleotide herein. In some embodiments, the method comprises selecting an individual having a marker (e.g., a biomarker) for a disease, disorder or condition associated with expression of a heart target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a heart target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of a heart target gene with an oligonucleotide provided herein. In some embodiments, the disclosure provides methods of treating or attenuating the onset or progression of a disease, disorder or condition associated with expression of a heart target gene using the oligonucleotides provided herein. In some embodiments, the disclosure provides methods to achieve one or more therapeutic benefits in a subject having a disease, disorder or condition associated with expression of a heart target gene using the oligonucleotides provided herein.

In some embodiments of the methods herein, the subject is treated by administering a therapeutically effective amount of any one or more of the oligonucleotides provided herein. In some embodiments, treatment comprises reducing expression of a heart target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of a heart target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of a heart target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of a heart target is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a heart target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a heart target gene such that an amount or level of protein encoded by the heart target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by a heart target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a heart target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ, blood or a fraction thereof (e.g., plasma), a tissue (e.g., heart tissue), a sample (e.g., heart biopsy sample), or any other biological material obtained or isolated from the subject. In some embodiments, expression of a heart target gene, an amount or level of target gene mRNA, an amount or level of protein encoded by the target gene, an amount or level of target gene activity, or any combination thereof, is reduced in more than one type of cell, more than one groups of cells, more than one organ (e.g., heart and one or more other organ(s)), more than one fraction of blood (e.g., plasma and one or more other blood fraction(s)), more than one type of tissue (e.g., heart tissue and one or more other type(s) of tissue), more than one type of sample (e.g., heart biopsy sample and one or more other type(s) of biopsy sample) obtained or isolated from the subject.

In some embodiments, the heart target gene may be a target gene from any mammal, such as a human. Any heart gene may be silenced according to the method described herein. In some embodiments, an oligonucleotide herein, or a composition thereof, is administered via subcutaneous or intravenous administration.

### Kits

In some embodiments, the disclosure provides a kit comprising a lipid-conjugated RNAi oligonucleotide herein, or a composition thereof, described herein, and instructions for use. In some embodiments, the kit comprises a lipid-conjugated RNAi oligonucleotide herein, or a composition thereof, described herein, and a package insert containing instructions for use of the kit and/or any component thereof. In some embodiments, the kit comprises, in a suitable container, a lipid-conjugated RNAi oligonucleotide herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the lipid-conjugated RNAi oligonucleotide herein, or a composition thereof, is placed, and in some instances, suitably aliquoted. In some embodiments where an additional component is provided, the kit contains additional containers into which this component is placed. The kits can also include a means for containing a lipid-conjugated RNAi oligonucleotide herein, or a composition thereof, and any other reagent in close confinement for commercial sale. Such containers may include injection or blow-molded plastic containers into which the desired vials are retained. Containers and/or kits can include labeling with instructions for use and/or warnings.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in one or more tissues or cells in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in the CNS (e.g., neuronal target gene) in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in ocular tissue in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in macrophages in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in macrophages of the liver in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in adipose tissue in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in cardiac tissue in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in muscle tissue (e.g., skeletal muscle) in a subject in need thereof.

In some embodiments, a kit comprises a lipid-conjugated RNAi oligonucleotide herein, or a composition thereof, described herein, and a pharmaceutically acceptable carrier, or a pharmaceutical composition comprising the lipid-conjugated RNAi oligonucleotide and instructions for treating or delaying progression of a disease, disorder or condition associated with expression of a target gene expressed in adrenal tissue in a subject in need thereof.

### Definitions

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, GUIDE TO MOLECULAR CLONING TECHNIQUES, METHODS IN ENZYMOLOGY, volume 152, (Academic Press, Inc., San Diego, Calif.) (“Berger”); Sambrook et al., MOLECULAR CLONING—A LABORATORY MANUAL, 2d ed., Vol. 1-3, Cold Spring Harbor Laboratory, Cold Spring Harbor, 1989 (“Sambrook”) and CURRENT PROTOCOLS IN MOLECULAR BIOLOGY, F. M. Ausubel et al., eds., CURRENT PROTOCOLS, A JOINT VENTURE BETWEEN GREENE PUBLISHING ASSOCIATES, INC. AND JOHN WILEY AND SONS, INC., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al., (1987) U.S. Pat. No. 4,683,202; Innis et al., eds. (1990); PCR PROTOCOLS: A GUIDE TO METHODS AND APPLICATIONS (Academic Press Inc. San Diego, Calif.) (“Innis”); Arnheim and Levinson (Oct. 1, 1990) CandEN 36-47; J. NIH RES. (1991) 3:81-94; Kwoh et al., (1989) PROC. NATL. ACAD. SCI. USA 86:1173; Guatelli et al (1990) PROC. NAT'L. ACAD. SCI. USA 87:1874; Lomell et al., (1989) J. CLIN. CHEM 35:1826; Landegren et al., (1988) SCIENCE 241:1077-80; Van Brunt (1990) BIOTECHNOLOGY 8:291-94; Wu and Wallace (1989) GENE 4:560; Barringer et al., (1990) GENE 89:117; and, Sooknanan and Malek (1995) BIOTECHNOLOGY 13:563-564. Improved methods for cloning in vitro amplified nucleic acids are described in Wallace et al., U.S. Pat. No. 5,426,039. Improved methods for amplifying large nucleic acids by PCR are summarized in Cheng et al., (1994) NATURE 369:684-85 and the references cited therein, in which PCR amplicons of up to 40 kb are generated.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one value, and/or to “about” another value. When such a range is expressed, another embodiment includes from the one value and/or to the other value. Similarly, when values are expressed as approximations, by use of the antecedent “about,” it will be understood that the value forms another embodiment. It will be further understood that the endpoints of each of the ranges are significant both in relation to the other endpoint, and independently of the other endpoint. It is also understood that there are several values disclosed herein, and that each value is also herein disclosed as “about” that value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in several different formats, and that this data, represents endpoints and starting points, and ranges for any combination of the data points. For example, if a particular datapoint “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used here, the term “amount” refers to an absolute amount (e.g., an absolute amount of mRNA or protein), a relative amount (e.g., a relative amount of target mRNA as measured by PCR assay or protein), or a concentration (e.g. a concentration of lipid-conjugated RNA in a composition), whether the amount referred to in a given instance refers to an absolute amount, concentration, or both, will be clear to the skilled artisan based on the context provided herein.

As used herein, “bicyclic nucleotide” refers to a nucleotide comprising a bicyclic sugar moiety.

As used herein “bicyclic sugar moiety” refers to a modified sugar moiety comprising a 4 to 7 membered ring (including but not limited to a furanosyl) comprising a bridge connecting two atoms of the 4 to 7 membered ring to form a second ring, resulting in a bicyclic structure. Typically, the 4 to 7 membered ring is a sugar. In some embodiments, the 4-to-7-member ring is a furanosyl. In certain embodiments, the bridge connects the 2′-carbon and the 4′-carbon of the furanosyl.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA” or “dsRNA” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect. As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “melting temperature” or “Tm” means the temperature at which the two strands of a duplex nucleic acid separate. Tₘ is often used as a measure of duplex stability or the binding affinity of two strands of complementary nucleic acids or portions thereof. Tₘ can be measured by using the UV spectrum to determine the formation and breakdown (melting) of hybridization. Base stacking, which occurs during hybridization, is accompanied by a reduction in UV absorption (hypochromicity). Consequently, a reduction in UV absorption indicates a higher Tₘ.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein “neuronal mRNA” and “neuronal gene” refers to any gene, mRNA, and/or protein encoded/expressed by a gene in neurons of the central nervous system. In some embodiments, the neuronal mRNA or neuronal gene is predominantly expressed in neurons relative to other cell types, e.g., other cell types of the CNS.

As used herein “ocular mRNA” and “ocular gene” refers to any gene, mRNA, and/or protein encoded/expressed by a gene in a cell in ocular tissue. In some embodiments, the ocular mRNA or ocular gene is predominantly expressed in cells of ocular tissue relative to other cell types, e.g., cell types of other organs.

As used herein “macrophage mRNA” and “macrophage gene” refers to any gene, mRNA, and/or protein encoded/expressed by a gene in a macrophage in a tissue, e.g., liver. In some embodiments, the macrophage mRNA or macrophage gene is predominantly expressed in macrophages relative to other cell types, e.g., non-macrophage immune cells.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded (dsRNA) is an RNAi oligonucleotide.

The terms “lipid-conjugated RNAi oligonucleotide” and “oligonucleotide-ligand conjugate” are used interchangeably and refer to an oligonucleotide comprising one or more nucleotides conjugated with one or more targeting ligands (e.g., lipid).

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In some embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al., (2015) NUCLEIC ACIDS RES. 43:2993-3011).

As used herein, “reduced expression” of a target gene refers to a decrease in the amount or level of RNA transcript (e.g., target mRNA) or protein encoded by the target gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide or conjugate herein (e.g., an lipid-conjugated RNAi oligonucleotide comprising an antisense strand having a nucleotide sequence that is complementary to a nucleotide sequence comprising a target mRNA) may result in a decrease in the amount or level of target mRNA, protein encoded by a target gene, and/or target gene activity (e.g., via inactivation and/or degradation of target mRNA by the RNAi pathway) when compared to a cell that is not treated with the double-stranded oligonucleotide. Similarly, and as used herein, “reducing expression” refers to an act that results in reduced expression of a target gene.

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base. As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA. As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule or “moiety” (e.g., a carbohydrate, amino sugar, cholesterol, polypeptide, or lipid) that selectively binds to a cognate molecule (e.g., a receptor) of a tissue or cell of interest and/or that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tn of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al., (1990) NATURE 346:680-82; Heus and Pardi (1991) SCIENCE 253:191-94). In some embodiments, a tetraloop comprises or consists of 3 to 6 nucleotides and is typically 4 to 5 nucleotides. In some embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden ((1985) NUCLEIC ACIDS RES. 13:3021-3030). For example, the letter “N” may be used to mean that any base may be in that position, the letter “R” may be used to show that A (adenine) or G (guanine) may be in that position, and “B” may be used to show that C (cytosine), G (guanine), or T (thyminc) may be in that position. Examples of tetraloops include the UNCG family of tetraloops (e.g., UUCG), the GNRA family of tetraloops (e.g., GAAA), and the CUUG tetraloop (Woese et al., (1990) PROC. NATL. ACAD. SCI. USA 87:8467-71; Antao et al., (1991) NUCLEIC ACIDS RES. 19:5901-05). Examples of DNA tetraloops include the d (GNNA) family of tetraloops (e.g., d (GTTA), the d (GNRA)) family of tetraloops, the d (GNAB) family of tetraloops, the d (CNNG) family of tetraloops, and the d (TNCG) family of tetraloops (e.g., d (TTCG)). (See, e.g., Nakano et al., (2002) BIOCHEM. 41:4281-92; Shinji et al., (2000) NIPPON KAGAKKAI KOEN YOKOSHU 78:731). In some embodiments, the tetraloop is contained within a nicked tetraloop structure.

As used herein, “Tm-increasing nucleotide” refers to a nucleotide that increases the melting temperature (Tₘ) of an oligonucleotide duplex as compared to the oligonucleotide duplex without the Tₘ-increasing nucleotide. Tₘ-increasing nucleotides include, but are not limited to, bicyclic nucleotides, tricyclic nucleotides, a G-clamp, and analogues thereof, and hexitol nucleotides. Certain modified nucleotides having a modified sugar moiety, or a modified nucleobase can also be used to increase the Tₘ of an oligonucleotide duplex. As used herein, the term “Tₘ-increasing nucleotide” specifically excludes nucleotides modified at the 2′-position of the sugar moiety with 2′-OMe or 2′-F.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### Embodiments

Embodiment I-1. A double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to an astrocyte mRNA target sequence, and wherein the sense strand comprises at least one lipid moiety conjugated to a nucleotide of the sense strand.
Embodiment I-2. The oligonucleotide of Embodiment I-1, wherein the lipid moiety is selected from

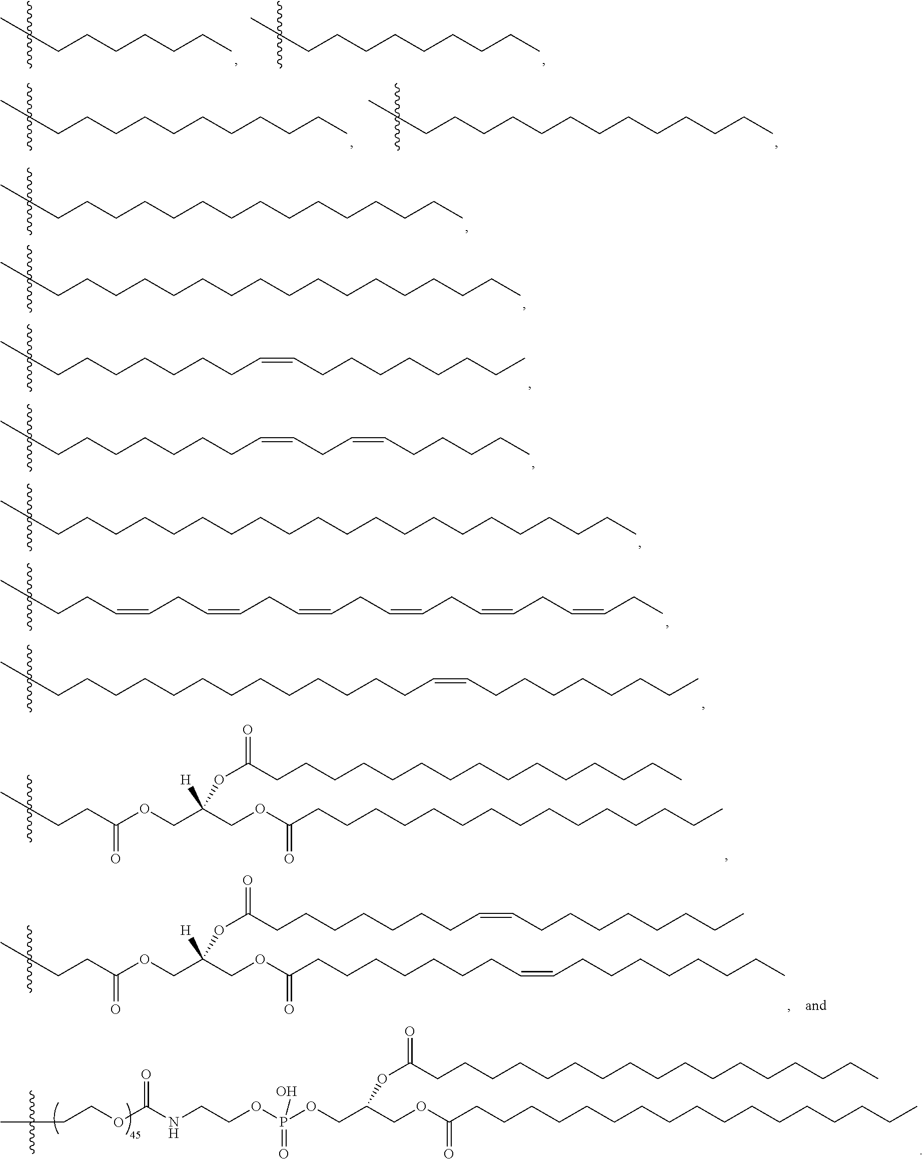

Embodiment I-3. The oligonucleotide of Embodiment I-1, wherein the lipid moiety is a hydrocarbon chain.
Embodiment I-4. The oligonucleotide of Embodiment I-3, wherein the hydrocarbon chain is a C8-C30 hydrocarbon chain.
Embodiment I-5. The oligonucleotide of Embodiment I-3 or 4, wherein the hydrocarbon chain is a C16 hydrocarbon chain.
Embodiment I-6. The oligonucleotide of Embodiment I-5 wherein the C16 hydrocarbon chain is represented by

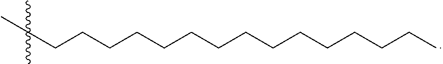 [SMILES: *CCCCCCCCCCCCCCC]

Embodiment I-7. The oligonucleotide of any one of Embodiments I-1-6, wherein the lipid moiety is conjugated to the 2′ carbon of the ribose ring of the nucleotide.
Embodiment I-8. The oligonucleotide of any one of Embodiments I-1-7, wherein the oligonucleotide is blunt ended.
Embodiment I-9. The oligonucleotide of Embodiment I-8, wherein the oligonucleotide is blunt ended at the 3′ terminus of the oligonucleotide.
Embodiment I-10. The oligonucleotide of any one of Embodiments I-1-7, wherein the oligonucleotide comprises a blunt end.
Embodiment I-11. The oligonucleotide of Embodiment I-10, wherein the blunt end comprises the 3′ end of the sense strand.
Embodiment I-12. The oligonucleotide of any one of Embodiments I-8-11, wherein the sense strand is 20-22 nucleotides.
Embodiment I-13. The oligonucleotide of Embodiment I-12, wherein the at least one lipid moiety is conjugated to a nucleotide at position 1, position 4, position 8, position 12, position 13, position 18, or position 20 of the sense strand, wherein positions are numbered 5′ to 3′.
Embodiment I-14. The oligonucleotide of Embodiment I-12, wherein the astrocyte mRNA target is expressed in the spinal cord, wherein the at least one lipid moiety is conjugated to a nucleotide at position 1, position 4, position 8, position 12, position 13, position 18 or position 20 of the sense strand, and wherein positions are numbered 5′ to 3′.
Embodiment I-15. The oligonucleotide of Embodiment I-12, wherein the astrocyte mRNA target is expressed in the medulla, wherein the at least one lipid moiety is conjugated to a nucleotide at position 1, position 4, position 8, position 12, position 13, position 18 or position 20 of the sense strand, and wherein positions are numbered 5′ to 3′.
Embodiment I-16. The oligonucleotide of Embodiment I-12, wherein the astrocyte mRNA target is expressed in the cerebellum, wherein the at least one lipid moiety is conjugated to a nucleotide at position 4, position 12, position 13, position 18 or position 20 of the sense strand, and wherein positions are numbered 5′ to 3′.
Embodiment I-17. The oligonucleotide of Embodiment I-12, wherein the astrocyte mRNA target is expressed in the hypothalamus, wherein the at least one lipid moiety is conjugated to a nucleotide at position 1, position 4, position 12, position 13, position 18 or position 20 of the sense strand, and wherein positions are numbered 5′ to 3′.
Embodiment I-18. The oligonucleotide of Embodiment I-12, wherein the astrocyte mRNA target is expressed in the frontal cortex, wherein the at least one lipid moiety is conjugated to a nucleotide at position 4 of the sense strand, and wherein positions are numbered 5′ to 3′.
Embodiment I-19. The oligonucleotide of any one of Embodiments I-1-7, wherein the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2.
Embodiment I-20. The oligonucleotide of any one of Embodiments I-1-7 and 19, wherein the sense strand is 36-38 nucleotides.
Embodiment I-21. The oligonucleotide of Embodiment I-20, wherein the astrocyte mRNA target is expressed in the spinal cord, wherein the at least one lipid moiety is conjugated to a nucleotide at position 1, position 4, position 8, position 12, position 13, position 18, position 20, position 23, position 28, position 29 or position 30 of the sense strand, and wherein positions are numbered 5′ to 3′.
Embodiment I-22. The oligonucleotide of Embodiment I-20, wherein the astrocyte mRNA target is expressed in the medulla, wherein the at least one lipid moiety is conjugated to a nucleotide at position 1, position 4, position 18, position 20, position 23, position 28, position 29 or position 30 of the sense strand, and wherein positions are numbered 5′ to 3′.
Embodiment I-23. The oligonucleotide of Embodiment I-20, wherein the astrocyte mRNA target is expressed in the cerebellum, wherein the at least one lipid moiety is conjugated to a nucleotide at position 1, position 4, position 23, position 28, or position 29 of the sense strand, and wherein positions are numbered 5′ to 3′.
Embodiment I-24. The oligonucleotide of Embodiment I-20, wherein the astrocyte mRNA target is expressed in the hypothalamus, wherein the at least one lipid moiety is conjugated to a nucleotide at position 1, position 4, position 12, position 13, position 18, position 20, position 23, position 28, position 29 or position 30 of the sense strand, and wherein positions are numbered 5′ to 3′.
Embodiment I-25. The oligonucleotide of Embodiment I-20, wherein the astrocyte mRNA target is expressed in the frontal cortex, wherein the at least one lipid moiety is conjugated to a nucleotide at position 23 of the sense strand, and wherein positions are numbered 5′ to 3′.
Embodiment I-26. The oligonucleotide of any one of Embodiments I-1-25, wherein the antisense strand is 22-24 nucleotides.
Embodiment I-27. The oligonucleotide of any one of Embodiments I-1-26, wherein the duplex region is 20-22 base pairs.
Embodiment I-28. The oligonucleotide of any one of Embodiments I-1-27, wherein the antisense strand comprises a 1-4 nucleotide overhang at the 3′ terminus.
Embodiment I-29. The oligonucleotide of Embodiment I-28, wherein the overhang comprises purine nucleotides.
Embodiment I-30. The oligonucleotide of Embodiment I-28 or 29, wherein the overhang sequence is 2 nucleotides in length.
Embodiment I-31. The oligonucleotide of Embodiment I-30, wherein the overhang is selected from AA, GG, AG, and GA.
Embodiment I-32. The oligonucleotide of Embodiment I-31, wherein the overhang is GG or AA.
Embodiment I-33. The oligonucleotide of Embodiment I-31, wherein the overhang is GG.
Embodiment I-34. The oligonucleotide of any one of Embodiments I-1-33, wherein the region of complementarity is complementary to at least 15 consecutive nucleotides of the astrocyte mRNA target sequence.
Embodiment I-35. The oligonucleotide of any one of Embodiments I-1-34, wherein the region of complementarity is complementary to 19 consecutive nucleotides of the astrocyte mRNA target sequence.
Embodiment I-36. The oligonucleotide of any one of Embodiments I-1-35, wherein the region of complementarity is fully complementary to the astrocyte mRNA target sequence.
Embodiment I-37. The oligonucleotide of any one of Embodiments I-1-35, wherein the region of complementarity is partially complementary to the astrocyte mRNA target sequence.
Embodiment I-38. The oligonucleotide of Embodiment I-37, wherein the region of complementarity comprises no more than four mismatches to the astrocyte mRNA target sequence.
Embodiment I-39. The oligonucleotide of any one of Embodiments I-1-38, wherein the oligonucleotide comprises at least one modified nucleotide.
Embodiment I-40. The oligonucleotide of Embodiment I-39, wherein the modified nucleotide comprises a 2′-modification.
Embodiment I-41. The oligonucleotide of Embodiment I-40, wherein each of the nucleotides of the sense strand and the antisense strand comprise a 2′-modification except the nucleotide of the sense strand conjugated to the at least one lipid moiety.
Embodiment I-42. The oligonucleotide of Embodiment I-40 or 41, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.
Embodiment I-43. The oligonucleotide of any one of Embodiments I-40-42, wherein about 10-20%, 10%, 11%, 12%, 13%, 14% 15%, 16%, 17%, 18%, 19% or 20% of the nucleotides of the sense strand comprise a 2′-fluoro modification.
Embodiment I-44. The oligonucleotide of any one of Embodiments I-40-43, wherein about 25-35%, 25%, 26%, 27%, 28%, 29%, 30%, 31%, 32%, 33%, 34% or 35% of the nucleotides of the antisense strand comprise a 2′-fluoro modification.
Embodiment I-45. The oligonucleotide of any one of Embodiments I-40-44, wherein about 25-35%, 25%, 26%, 27%, 28%, 29%, 30%, 31%, 32%, 33%, 34% or 35% of the nucleotides of the oligonucleotide comprise a 2′-fluoro modification.
Embodiment I-46. The oligonucleotide of any one of Embodiments I-40-45, wherein the sense strand comprises 20 nucleotides with positions 1-20 from 5′ to 3′, wherein each of positions 8-11 comprise a 2′-fluoro modification.
Embodiment I-47. The oligonucleotide of any one of Embodiments I-40-45, wherein the sense strand comprises 20 nucleotides with positions 1-20 from 5′ to 3′, wherein each of positions 9-11 comprise a 2′-fluoro modification.
Embodiment I-48. The oligonucleotide of any one of Embodiments I-40-45, wherein the sense strand comprises 36 nucleotides with positions 1-36 from 5′ to 3′, wherein each of positions 8-11 comprise a 2′-fluoro modification.
Embodiment I-49. The oligonucleotide of any one of Embodiments I-40-45, wherein the sense strand comprises 36 nucleotides with positions 1-36 from 5′ to 3′, wherein each of positions 9-11 comprise a 2′-fluoro modification.
Embodiment I-50. The oligonucleotide of any one of Embodiments I-40-49, wherein the antisense strand comprises 22 nucleotides with positions 1-22 from 5′ to 3′, and wherein each of positions 2, 3, 4, 5, 7, 10 and 14 comprise a 2′-fluoro modification.
Embodiment I-51. The oligonucleotide of any one of Embodiments I-41-50, wherein the remaining nucleotides comprise a 2′-O-methyl modification except the nucleotide of the sense strand conjugated to the at least one lipid moiety.
Embodiment I-52. The oligonucleotide of any one of the preceding Embodiments, wherein the oligonucleotide comprises at least one modified internucleotide linkage.
Embodiment I-53. The oligonucleotide of Embodiment I-52, wherein the at least one modified internucleotide linkage is a phosphorothioate linkage.
Embodiment I-54. The oligonucleotide of Embodiment I-53, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.
Embodiment I-55. The oligonucleotide of Embodiment I-53 or 54, wherein the antisense strand is 22 nucleotides in length, and wherein the antisense strand comprises a phosphorothioate linkage between positions 20 and 21 and between positions 21 and 22, wherein positions are numbered 1-22 from 5′ to 3′.
Embodiment I-56. The oligonucleotide of any one of Embodiments I-53-55, wherein the sense strand comprises a phosphorothioate linkage between position 1 and 2, wherein positions are numbered 1-2 from 5′ to 3′.
Embodiment I-57. The oligonucleotide of any one of Embodiments I-53-56, wherein the sense strand is 20 nucleotides in length, and wherein the sense strand comprises a phosphorothioate linkage between positions 18 and 19, and between positions 19 and 20, wherein positions are numbered 1-22 from 5′ to 3′.
Embodiment I-58. The oligonucleotide of any one of Embodiments I-1-57, wherein the antisense strand comprises a phosphorylated nucleotide at the 5′ terminus, wherein the phosphorylated nucleotide is selected from uridine and adenosine.
Embodiment I-59. The oligonucleotide of Embodiment I-58, wherein the phosphorylated nucleotide is uridine.
Embodiment I-60. The oligonucleotide of any one of the preceding Embodiments, wherein the 4′-carbon of the sugar of the 5′-nucleotide of the antisense strand comprises a phosphate analog.
Embodiment I-61. The oligonucleotide of Embodiment I-60, wherein the phosphate analog is oxymethyl phosphonate, vinyl phosphonate or malonyl phosphonate.
Embodiment I-62. The oligonucleotide of any one of Embodiments I-1-61, wherein the region of complementary is fully complementary to the astrocyte mRNA target sequence at nucleotide positions 2-8 of the antisense strand, wherein nucleotide positions are numbered 5′ to 3′.
Embodiment I-63. The oligonucleotide of any one of Embodiments I-1-61, wherein the region of complementary is fully complementary to the astrocyte mRNA target sequence at nucleotide positions 2-11 of the antisense strand, wherein nucleotide positions are numbered 5′ to 3′.
Embodiment I-64. The oligonucleotide of any one of Embodiments I-1-63, wherein the oligonucleotide is a Dicer substrate.
Embodiment I-65. The oligonucleotide of any one of Embodiments I-1-63, wherein the oligonucleotide is a Dicer substrate that, upon endogenous Dicer processing, yields double-stranded nucleic acids of 19-21 nucleotides in length capable of reducing an astrocyte mRNA expression in a mammalian cell.
Embodiment I-66. The oligonucleotide of any one of Embodiments I-1-65, wherein the astrocyte mRNA target sequence is located in a region of the central nervous system (CNS).
Embodiment I-67. The oligonucleotide of Embodiment I-66, wherein the region of the CNS is selected from the spinal cord, lumbar spinal cord, thoracic spinal cord, cervical spinal cord, medulla, hippocampus, cerebellum, hypothalamus, frontal cortex, and a combination thereof.
Embodiment I-68. The oligonucleotide of any one of Embodiments I-1-67, wherein the oligonucleotide reduces expression of a target mRNA in an astrocyte or population of astrocytes in vitro and/or in vivo.
Embodiment I-69. A pharmaceutical composition comprising the oligonucleotide of any one of Embodiments I-1-68, and a pharmaceutically acceptable carrier, delivery agent or excipient.
Embodiment I-70. A method for treating a subject having a disease, disorder or condition associated with expression of an astrocyte mRNA, the method comprising administering to the subject a therapeutically effective amount of the oligonucleotide of any one of Embodiments I-1-68 or the pharmaceutical composition of Embodiment I-69, thereby treating the subject.
Embodiment I-71. A method of delivering an oligonucleotide to an astrocyte or a population of astrocytes in a subject, the method comprising administering the pharmaceutical composition of Embodiment I-69 to the subject.
Embodiment I-72. The method of Embodiment I-71, wherein the astrocyte or a population of astrocytes is located in a region of the CNS.
Embodiment I-73. The method of Embodiment I-72, wherein the region of the CNS is selected from the spinal cord, lumbar spinal cord, thoracic spinal cord, cervical spinal cord, medulla, hippocampus, cerebellum, hypothalamus, frontal cortex, and a combination thereof.
Embodiment I-74. A method for reducing expression of an astrocyte mRNA in a cell, a population of cells or a subject, the method comprising the step of:
i. contacting the cell or the population of cells with the oligonucleotide of any one of Embodiments I-1 to 68, or the pharmaceutical composition of Embodiment I-69, optionally wherein the cell or population of cells is an astrocyte or a population of astrocytes; or
ii. administering to the subject the oligonucleotide of any one of Embodiments I-1 to 68, or the pharmaceutical composition of Embodiment I-69.
Embodiment I-75. The method of Embodiment I-74, wherein reducing expression of the astrocyte mRNA comprises reducing an amount or level of mRNA, an amount or level of protein, or both.
Embodiment I-76. The method of Embodiment I-74 or 75, wherein the subject has a disease, disorder or condition associated with expression of the astrocyte mRNA.
Embodiment I-77. The method of any one of Embodiments I-74-76, wherein the cell or population of cells is located in a region of the CNS.
Embodiment I-78. The method of Embodiment I-77, wherein the region of the CNS is selected from the spinal cord, lumbar spinal cord, thoracic spinal cord, cervical spinal cord, medulla, hippocampus, cerebellum, hypothalamus, frontal cortex, and a combination thereof.
Embodiment I-79. The method of any one of Embodiments I-70-78, wherein administering is intrathecal.
Embodiment I-80. A method of reducing expression of a target mRNA expressed in an astrocyte in a tissue of the CNS of a subject, comprising administering to the subject a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a target sequence in the target mRNA, and wherein the sense strand comprises at least one lipid moiety conjugated to a nucleotide of the sense strand.
Embodiment I-81. The method of Embodiment I-80, wherein the lipid moiety is a C16 hydrocarbon.
Embodiment I-82. The method of any one of Embodiments I-80-81, wherein the oligonucleotide is blunt-ended at the 3′ terminus of the oligonucleotide.
Embodiment I-83. The method of Embodiment I-82, wherein the sense strand is 22-24 nucleotides.
Embodiment I-84. The method of Embodiment I-83, wherein the tissue is the spinal cord, wherein the at least one lipid moiety is conjugated to a nucleotide at position 1, position 4, position 8, position 12, position 13, position 18 or position 20 of the sense strand, and wherein positions are numbered 5′ to 3′
Embodiment I-85. The method of Embodiment I-83, wherein the tissue is the medulla, wherein the at least one lipid moiety is conjugated to a nucleotide at position 1, position 4, position 8, position 12, position 13, position 18 or position 20 of the sense strand, and wherein positions are numbered 5′ to 3′.
Embodiment I-86. The method of Embodiment I-83, wherein the tissue is the cerebellum, wherein the at least one lipid moiety is conjugated to a nucleotide at position 4, position 12, position 13, position 18 or position 20 of the sense strand, and wherein positions are numbered 5′ to 3′.
Embodiment I-87. The method of Embodiment I-83, wherein the tissue is the hypothalamus, wherein the at least one lipid moiety is conjugated to a nucleotide at position 1, position 4, position 12, position 13, position 18 or position 20 of the sense strand, and wherein positions are numbered 5′ to 3.
Embodiment I-88. The method of Embodiment I-83, wherein the tissue is the frontal cortex, wherein the at least one lipid moiety is conjugated to a nucleotide at position 4 of the sense strand, and wherein positions are numbered 5′ to 3′.
Embodiment I-89. The method of any one of Embodiments I-80-81, wherein the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2.
Embodiment I-90. The method of Embodiment I-89, wherein the sense strand is 36-38 nucleotides.
Embodiment I-91. The method of Embodiment I-90, wherein the tissue is the spinal cord, wherein the at least one lipid moiety is conjugated to a nucleotide at position 1, position 4, position 8, position 12, position 13, position 18, position 20, position 23, position 28, position 29 or position 30 of the sense strand, and wherein positions are numbered 5′ to 3′.
Embodiment I-92. The method of Embodiment I-90, wherein the tissue is the medulla, wherein the at least one lipid moiety is conjugated to a nucleotide at position 1, position 4, position 18, position 20, position 23, position 28, position 29 or position 30 of the sense strand, and wherein positions are numbered 5′ to 3′.
Embodiment I-93. The method of Embodiment 1-90, wherein the tissue is the cerebellum, wherein the at least one lipid moiety is conjugated to a nucleotide at position 1, position 4, position 23, position 28, or position 29 of the sense strand, and wherein positions are numbered 5′ to 3′.
Embodiment I-94. The method of Embodiment I-90, wherein the tissue is the hypothalamus, wherein the at least one lipid moiety is conjugated to a nucleotide at position 1, position 4, position 12, position 13, position 18, position 20, position 23, position 28, position 29 or position 30 of the sense strand, and wherein positions are numbered 5′ to 3′.
Embodiment I-95. The method of Embodiment I-90, wherein the tissue is the frontal cortex, wherein the at least one lipid moiety is conjugated to a nucleotide at position 23 of the sense strand, and wherein positions are numbered 5′ to 3′.
Embodiment I-96. The method of any one of Embodiments I-70-95, wherein a single dose of the oligonucleotide or pharmaceutical composition reduces expression of the astrocyte mRNA for at least 4 weeks, at least 8 weeks, at least 12 weeks, at least 23 weeks, at least 26 weeks, or at least 29 weeks.
Embodiment I-97. The method of any one of Embodiments I-70-95, wherein a single dose of the oligonucleotide or pharmaceutical composition reduces expression of the astrocyte mRNA for up to one year.
Embodiment I-98. A kit comprising the oligonucleotide of any one of Embodiments I-1-68, an optional pharmaceutically acceptable carrier, and a package insert comprising instructions for administration to a subject having a disease, disorder or condition associated with expression of an astrocyte mRNA.
Embodiment I-99. The kit of Embodiment I-98, wherein the package insert comprises instructions for intrathecal administration.
Embodiment I-100. Use of the oligonucleotide of any one of Embodiments I-1-68 or the pharmaceutical composition of Embodiment I-69, in the manufacture of a medicament for the treatment of a disease, disorder or condition associated with expression of an astrocyte mRNA.
Embodiment I-101. The oligonucleotide of any one of Embodiments I-1-68 or the pharmaceutical composition of Embodiment I-69, for use, or adaptable for use, in the treatment of a disease, disorder or condition associated with expression of an astrocyte mRNA.
Embodiment II-1. A double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to an oligodendrocyte mRNA target sequence, and wherein the sense strand comprises at least one lipid moiety conjugated to a nucleotide of the sense strand.
Embodiment II-2. The oligonucleotide of Embodiment II-1, wherein the lipid moiety is selected from

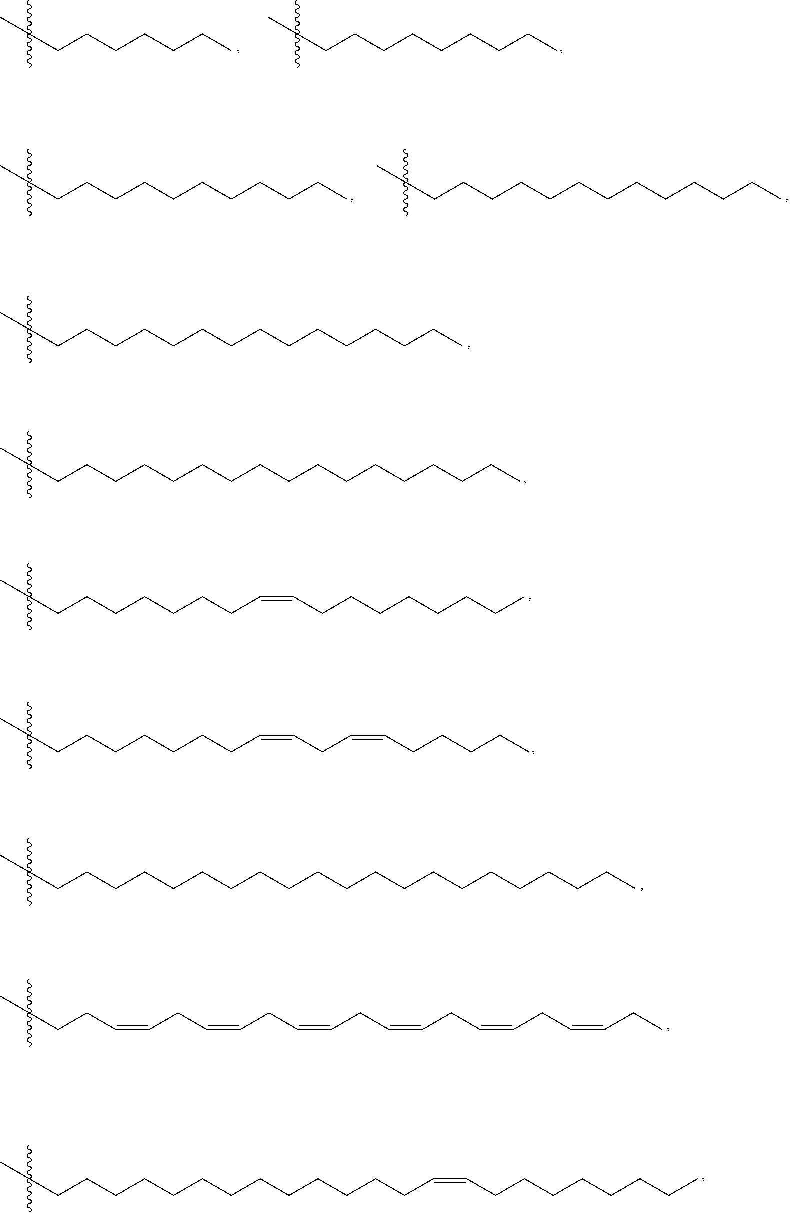

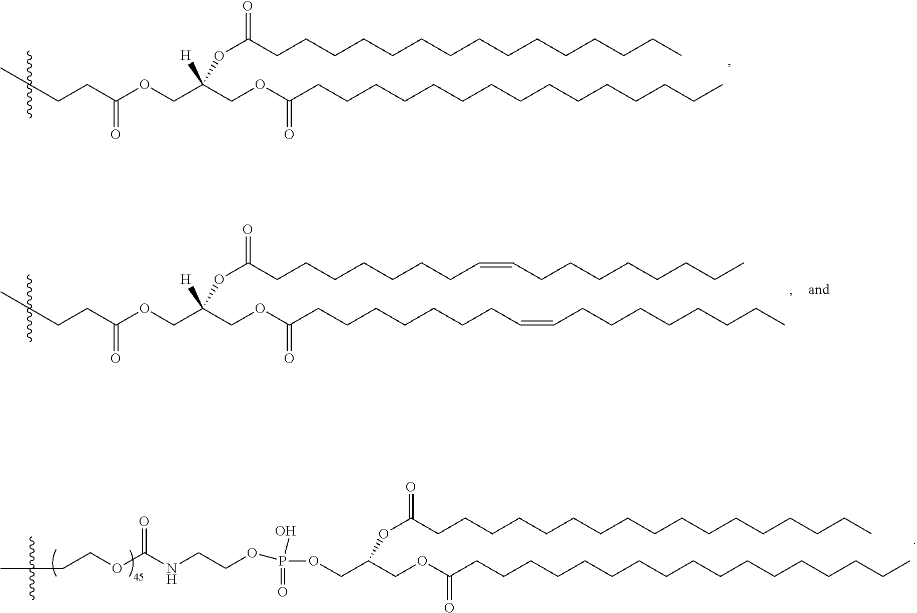

Embodiment II-3. The oligonucleotide of Embodiment II-1, wherein the lipid moiety is a hydrocarbon chain.
Embodiment II-4. The oligonucleotide of Embodiment II-3, wherein the hydrocarbon chain is a C8-C30 hydrocarbon chain.
Embodiment II-5. The oligonucleotide of Embodiment II-3 or 4, wherein the hydrocarbon chain is a C16 hydrocarbon chain.
Embodiment II-6. The oligonucleotide of Embodiment II-5 wherein the C16 hydrocarbon chain is represented by

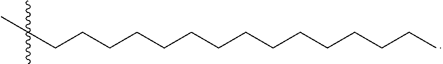 [SMILES: *CCCCCCCCCCCCCCC]

Embodiment II-7. The oligonucleotide of any one of Embodiments II-1-6, wherein the lipid moiety is conjugated to the 2′ carbon of the ribose ring of the nucleotide.
Embodiment II-8. The oligonucleotide of any one of Embodiments II-1-7, wherein the oligonucleotide is blunt ended.
Embodiment II-9. The oligonucleotide of Embodiment II-8, wherein the oligonucleotide is blunt ended at the 3′ terminus of the oligonucleotide.
Embodiment II-10. The oligonucleotide of any one of Embodiments II-1-7, wherein the oligonucleotide comprises a blunt end.
Embodiment II-11. The oligonucleotide of Embodiment II-11, wherein the blunt end comprises the 3′ terminus of the sense strand
Embodiment II-12. The oligonucleotide of any one of Embodiments II-8-11, wherein the sense strand is 20-22 nucleotides.
Embodiment II-13. The oligonucleotide of Embodiment II-12, wherein the at least one lipid moiety is conjugated to a nucleotide at position 1, position 4, position 8, position 12, position 13, position 18, or position 20 of the sense strand, wherein positions are numbered 5′ to 3′.
Embodiment II-14. The oligonucleotide of Embodiment II-12, wherein the oligodendrocyte mRNA target is expressed in the spinal cord, wherein the at least one lipid moiety is conjugated to a nucleotide at position 1, position 2, position 3, position 5, position 6, position 7, position 9, position 13, position 14, position 15, position 17, position 19, or position 20 of the sense strand, and wherein positions are numbered 5′ to 3′.
Embodiment II-15. The oligonucleotide of Embodiment II-12, wherein the oligodendrocyte mRNA target is expressed in the medulla, wherein the at least one lipid moiety is conjugated to a nucleotide at position 2, position 7, position 9, position 14, position 15, or position 19 of the sense strand, and wherein positions are numbered 5′ to 3′.
Embodiment II-16. The oligonucleotide of Embodiment II-12, wherein the oligodendrocyte mRNA target is expressed in the hippocampus, wherein the at least one lipid moiety is conjugated to a nucleotide at position 3 of the sense strand, and wherein positions are numbered 5′ to 3′.
Embodiment II-17. The oligonucleotide of Embodiment II-12, wherein the oligodendrocyte mRNA target is expressed in the frontal cortex, wherein the at least one lipid moiety is conjugated to a nucleotide at position 14 of the sense strand, and wherein positions are numbered 5′ to 3′.
Embodiment II-18. The oligonucleotide of Embodiment II-12, wherein the oligodendrocyte mRNA target is expressed in the hypothalamus, wherein the at least one lipid moiety is conjugated to a nucleotide at position 7 of the sense strand, and wherein positions are numbered 5′ to 3′.
Embodiment II-19. The oligonucleotide of any one of Embodiments II-1-7, wherein the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2.
Embodiment II-20. The oligonucleotide of any one of Embodiments II-1-7 and 19, wherein the sense strand is 36-38 nucleotides.
Embodiment II-21. The oligonucleotide of Embodiment II-19 or 20, wherein the at least one lipid moiety is conjugated to a nucleotide of the stem-loop or a nucleotide proximal to the stem-loop.
Embodiment II-22. The oligonucleotide of Embodiment II-21, wherein the nucleotide proximal to the stem-loop is located 1-3 nucleotides from the 5′ end of the stem-loop.
Embodiment II-23. The oligonucleotide of Embodiment II-20, wherein the oligodendrocyte mRNA target is expressed in the spinal cord, wherein the at least one lipid moiety is conjugated to a nucleotide at position 2, position 3, position 6, position 13, position 14, position 15, position 19, position 20, position 23, position 28, position 29 or position 30 of the sense strand, and wherein positions are numbered 5′ to 3′.
Embodiment II-24. The oligonucleotide of Embodiment II-20, wherein the oligodendrocyte mRNA target is expressed in the medulla, wherein the at least one lipid moiety is conjugated to a nucleotide at position 19, position 20, position 23 or position 28 of the sense strand, and wherein positions are numbered 5′ to 3′.
Embodiment II-25. The oligonucleotide of Embodiment II-20, wherein the oligodendrocyte mRNA target is expressed in the hippocampus, wherein the at least one lipid moiety is conjugated to a nucleotide at position 2 of the sense strand, and wherein positions are numbered 5′ to 3′.
Embodiment II-26. The oligonucleotide of Embodiment II-20, wherein the oligodendrocyte mRNA target is expressed in the frontal cortex, wherein the at least one lipid moiety is conjugated to a nucleotide at position 14, position 15, position 19, position 20, position 23, position 28, position 29 or position 30 of the sense strand, and wherein positions are numbered 5′ to 3′.
Embodiment II-27. The oligonucleotide of any one of Embodiments II-1-26, wherein the antisense strand is 22-24 nucleotides.
Embodiment II-28. The oligonucleotide of any one of Embodiments II-1-27, wherein the duplex region is 20-22 base pairs.
Embodiment II-29. The oligonucleotide of any one of Embodiments II-1-28, wherein the antisense strand comprises a 1-4 nucleotide overhang at the 3′ terminus.
Embodiment II-30. The oligonucleotide of Embodiment II-29, wherein the overhang comprises purine nucleotides.
Embodiment II-31. The oligonucleotide of Embodiment II-29 or 30, wherein the overhang sequence is 2 nucleotides in length.
Embodiment II-32. The oligonucleotide of Embodiment II-30 or 31, wherein the overhang is selected from AA, GG, AG, and GA.
Embodiment II-33. The oligonucleotide of Embodiment II-32, wherein the overhang is GG.
Embodiment II-34. The oligonucleotide of any one of Embodiments II-1-33, wherein the region of complementarity is complementary to at least 15 consecutive nucleotides of the oligodendrocyte mRNA target sequence.
Embodiment II-35. The oligonucleotide of any one of Embodiments II-1-34, wherein the region of complementarity is complementary to 19 consecutive nucleotides of the oligodendrocyte mRNA target sequence.
Embodiment II-36. The oligonucleotide of any one of Embodiments II-1-35, wherein the region of complementarity is fully complementary to the oligodendrocyte mRNA target sequence.
Embodiment II-37. The oligonucleotide of any one of Embodiments II-1-35, wherein the region of complementarity is partially complementary to the oligodendrocyte mRNA target sequence.
Embodiment II-38. The oligonucleotide of Embodiment II-37, wherein the region of complementarity comprises no more than four mismatches to the oligodendrocyte mRNA target sequence.
Embodiment II-39. The oligonucleotide of any one of Embodiments II-1-38, wherein the oligonucleotide comprises at least one modified nucleotide.
Embodiment II-40. The oligonucleotide of Embodiment II-39, wherein the modified nucleotide comprises a 2′-modification.
Embodiment II-41. The oligonucleotide of Embodiment II-40, wherein each of the nucleotides of the sense strand and the antisense strand comprise a 2′-modification except the nucleotide of the sense strand conjugated to the at least one lipid moiety.
Embodiment II-42. The oligonucleotide of Embodiment II-40 or 41, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.
Embodiment II-43. The oligonucleotide of any one of Embodiments II-40-42, wherein about 10-20%, 10%, 11%, 12%, 13%, 14% 15%, 16%, 17%, 18%, 19% or 20% of the nucleotides of the sense strand comprise a 2′-fluoro modification.
Embodiment II-44. The oligonucleotide of any one of Embodiments II-40-43, wherein about 25-35%, 25%, 26%, 27%, 28%, 29%, 30%, 31%, 32%, 33%, 34% or 35% of the nucleotides of the antisense strand comprise a 2′-fluoro modification.
Embodiment II-45. The oligonucleotide of any one of Embodiments II-40-44, wherein about 25-35%, 25%, 26%, 27%, 28%, 29%, 30%, 31%, 32%, 33%, 34% or 35% of the nucleotides of the oligonucleotide comprise a 2′-fluoro modification.
Embodiment II-46. The oligonucleotide of any one of Embodiments II-40-45, wherein the sense strand comprises 20 nucleotides with positions 1-20 from 5′ to 3′, wherein each of positions 8-11 comprise a 2′-fluoro modification.
Embodiment II-47. The oligonucleotide of any one of Embodiments II-40-45, wherein the sense strand comprises 20 nucleotides with positions 1-20 from 5′ to 3′, wherein each of positions 9-11 comprise a 2′-fluoro modification.
Embodiment II-48. The oligonucleotide of any one of Embodiments II-40-45, wherein the sense strand comprises 36 nucleotides with positions 1-36 from 5′ to 3′, wherein each of positions 8-11 comprise a 2′-fluoro modification.
Embodiment II-49. The oligonucleotide of any one of Embodiments II-40-45, wherein the sense strand comprises 36 nucleotides with positions 1-36 from 5′ to 3′, wherein each of positions 9-11 comprise a 2′-fluoro modification.
Embodiment II-50. The oligonucleotide of any one of Embodiments II-40-49, wherein the antisense strand comprises 22 nucleotides with positions 1-22 from 5′ to 3′, and wherein each of positions 2, 3, 4, 5, 7, 10 and 14 comprise a 2′-fluoro modification.
Embodiment II-51. The oligonucleotide of any one of Embodiments II-41-50, wherein the remaining nucleotides comprise a 2′-O-methyl modification except the nucleotide of the sense strand conjugated to the at least one lipid moiety.
Embodiment II-52. The oligonucleotide of any one of the preceding Embodiments, wherein the oligonucleotide comprises at least one modified internucleotide linkage.
Embodiment II-53. The oligonucleotide of Embodiment II-52, wherein the at least one modified internucleotide linkage is a phosphorothioate linkage.
Embodiment II-54. The oligonucleotide of Embodiment II-53, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.
Embodiment II-55. The oligonucleotide of Embodiment II-53 or 54, wherein the antisense strand is 22 nucleotides in length, and wherein the antisense strand comprises a phosphorothioate linkage between positions 20 and 21 and between positions 21 and 22, wherein positions are numbered 1-22 from 5′ to 3′.
Embodiment II-56. The oligonucleotide of any one of Embodiments II-53-55, wherein the sense strand comprises a phosphorothioate linkage between position 1 and 2, wherein positions are numbered 1-2 from 5′ to 3′.
Embodiment II-57. The oligonucleotide of any one of Embodiments II-53-56, wherein the sense strand is 20 nucleotides in length, and wherein the sense strand comprises a phosphorothioate linkage between positions 18 and 19, and between positions 19 and 20, wherein positions are numbered 1-22 from 5′ to 3′.
Embodiment II-58. The oligonucleotide of any one of Embodiments II-1-57, wherein the antisense strand comprises a phosphorylated nucleotide at the 5′ terminus, wherein the phosphorylated nucleotide is selected from uridine and adenosine.
Embodiment II-59. The oligonucleotide of Embodiment II-58, wherein the phosphorylated nucleotide is uridine.
Embodiment II-60. The oligonucleotide of any one of the preceding Embodiments, wherein the 4′-carbon of the sugar of the 5′-nucleotide of the antisense strand comprises a phosphate analog.
Embodiment II-61. The oligonucleotide of Embodiment II-60, wherein the phosphate analog is oxymethyl phosphonate, vinyl phosphonate or malonyl phosphonate.
Embodiment II-62. The oligonucleotide of any one of Embodiments II-1-61, wherein the region of complementary is fully complementary to the oligodendrocyte mRNA target sequence at nucleotide positions 2-8 of the antisense strand, wherein nucleotide positions are numbered 5′ to 3′.
Embodiment II-63. The oligonucleotide of any one of Embodiments II-1-61, wherein the region of complementary is fully complementary to the oligodendrocyte mRNA target sequence at nucleotide positions 2-11 of the antisense strand, wherein nucleotide positions are numbered 5′ to 3′.
Embodiment II-64. The oligonucleotide of any one of Embodiments II-1-63, wherein the oligonucleotide is a Dicer substrate.
Embodiment II-65. The oligonucleotide of any one of Embodiments II-1-63, wherein the oligonucleotide is a Dicer substrate that, upon endogenous Dicer processing, yields double-stranded nucleic acids of 19-21 nucleotides in length capable of reducing a oligodendrocyte mRNA expression in a mammalian cell.
Embodiment II-66. The oligonucleotide of any one of Embodiments II-1-65, wherein the oligodendrocyte mRNA target sequence is located in a region of the central nervous system (CNS).
Embodiment II-67. The oligonucleotide of Embodiment II-66, wherein the region of the CNS is selected from the frontal cortex, spinal cord, lumbar spinal cord, cervical spinal cord, thoracic spinal cord, medulla, cerebellum, hypothalamus, hippocampus, and a combination thereof.
Embodiment II-68. The oligonucleotide of any one of Embodiments II-1-67, wherein the oligonucleotide reduces expression of a target mRNA in an oligodendrocyte or population of oligodendrocytes in vitro and/or in vivo.
Embodiment II-69. A pharmaceutical composition comprising the oligonucleotide of any one of Embodiments II-1-68, and a pharmaceutically acceptable carrier, delivery agent or excipient.
Embodiment II-70. A method for treating a subject having a disease, disorder or condition associated with expression of an oligodendrocyte mRNA, the method comprising administering to the subject a therapeutically effective amount of the oligonucleotide of any one of Embodiments II-1-68 or the pharmaceutical composition of Embodiment II-69, thereby treating the subject.
Embodiment II-71. A method of delivering an oligonucleotide to an oligodendrocyte or a population of oligodendrocytes in a subject, the method comprising administering the pharmaceutical composition of Embodiment II-69 to the subject.
Embodiment II-72. The method of Embodiment II-71, wherein the oligodendrocyte or a population of oligodendrocytes is located in a region of the CNS.
Embodiment II-73. The method of Embodiment II-72 wherein the region of the CNS is selected from the frontal cortex, spinal cord, lumbar spinal cord, cervical spinal cord, thoracic spinal cord, medulla, cerebellum, hypothalamus, hippocampus, and a combination thereof.
Embodiment II-74. A method for reducing expression of an oligodendrocyte mRNA in a cell, a population of cells or a subject, the method comprising the step of:
i. contacting the cell or the population of cells with the oligonucleotide of any one of Embodiments II-1 to 68, or the pharmaceutical composition of Embodiment II-69, optionally wherein the cell or population of cells is an oligodendrocyte or a population of oligodendrocytes; or
ii. administering to the subject the oligonucleotide of any one of Embodiments II-1 to 68, or the pharmaceutical composition of Embodiment II-69.
Embodiment II-75. The method of Embodiment II-74, wherein reducing expression of the oligodendrocyte mRNA comprises reducing an amount or level of mRNA, an amount or level of protein, or both.
Embodiment II-76. The method of Embodiment II-74 or 75, wherein the subject has a disease, disorder or condition associated with expression of the oligodendrocyte mRNA.
Embodiment II-77. The method of any one of Embodiments II-74-76, wherein the cell or population of cells is located in a region of the CNS.
Embodiment II-78. The method of Embodiment II-77, wherein the region of the CNS is selected from the frontal cortex, spinal cord, lumbar spinal cord, cervical spinal cord, thoracic spinal cord, medulla, cerebellum, hypothalamus, hippocampus, and a combination thereof.
Embodiment II-79. The method of any one of Embodiments II-70-78, wherein administering is intrathecal.
Embodiment II-80. A method of reducing expression of a target mRNA expressed in an oligodendrocyte in a tissue of the CNS of a subject, comprising administering to the subject a double-stranded oligonucleotide comprising an antisense strand of 15-30 nucleotides in length and a sense strand of 15-50 nucleotides in length, wherein the antisense and sense strands form a duplex region of 15-30 base pairs, wherein the antisense strand comprises a region of complementarity to a target sequence in the target mRNA, and wherein the sense strand comprises at least one lipid moiety conjugated to a nucleotide of the sense strand.
Embodiment II-81. The method of Embodiment II-80, wherein the lipid moiety is a C16 hydrocarbon.
Embodiment II-82. The method of any one of Embodiments II-80-81, wherein the oligonucleotide is blunt-ended at the 3′ terminus of the oligonucleotide.
Embodiment II-83. The method of Embodiment II-82, wherein the sense strand is 22-24 nucleotides.
Embodiment II-84. The method of Embodiment II-83, wherein the tissue is the spinal cord, wherein the at least one lipid moiety is conjugated to a nucleotide at position 1, position 2, position 3, position 5, position 6, position 7, position 9, position 13, position 14, position 15, position 17, position 19, or position 20 of the sense strand, and wherein positions are numbered 5′ to 3′
Embodiment II-85. The method of Embodiment II-83, wherein the tissue is the medulla, wherein the at least one lipid moiety is conjugated to a nucleotide at position 2, position 7, position 14, position 15, or position 19 of the sense strand, and wherein positions are numbered 5′ to 3′.
Embodiment II-86. The method of Embodiment II-83, wherein the tissue is the hippocampus, wherein the at least one lipid moiety is conjugated to a nucleotide at position 3 of the sense strand, and wherein positions are numbered 5′ to 3′.
Embodiment II-87. The method of Embodiment II-83, wherein the tissue is the frontal cortex, wherein the at least one lipid moiety is conjugated to a nucleotide at position 14 of the sense strand, and wherein positions are numbered 5′ to 3′.
Embodiment II-88. The method of Embodiment II-83, wherein the tissue is the hypothalamus, wherein the at least one lipid moiety is conjugated to a nucleotide at position 7 of the sense strand, and wherein positions are numbered 5′ to 3′.
Embodiment II-89. The method of any one of Embodiments II-80-81, wherein the sense strand comprises a stem-loop at the 3′ end, wherein the stem-loop comprises a nucleotide sequence represented by the formula: 5′-S1-L-S2-3′, wherein S1 is complementary to S2, and wherein L forms a loop between S1 and S2.
Embodiment II-90. The method of any one of Embodiments II-80-81 and 89, wherein the sense strand is 36-38 nucleotides.
Embodiment II-91. The method of Embodiment II-89 or 90, wherein the at least one lipid moiety is conjugated to a nucleotide of the stem-loop or a nucleotide proximal to the stem-loop.
Embodiment II-92. The method of Embodiment II-91, wherein the nucleotide proximal to the stem-loop is located 1-3 nucleotides from the 5′ end of the stem-loop.
Embodiment II-93. The method of Embodiment II-90, wherein the tissue is the spinal cord, wherein the at least one lipid moiety is conjugated to a nucleotide at position 2, position 3, position 6, position 13, position 14, position 15, position 19, position 20, position 23, position 28, position 29 or position 30 of the sense strand, and wherein positions are numbered 5′ to 3′.
Embodiment II-94. The method of Embodiment II-90, wherein the tissue is the medulla, wherein the at least one lipid moiety is conjugated to a nucleotide at position 19, position 20, position 23 or position 28 of the sense strand, and wherein positions are numbered 5′ to 3′.
Embodiment II-95. The method of Embodiment II-90, wherein the tissue is the hippocampus, wherein the at least one lipid moiety is conjugated to a nucleotide at position 2 of the sense strand, and wherein positions are numbered 5′ to 3′.
Embodiment II-96. The method of Embodiment II-90, wherein the tissue is the frontal cortex, wherein the at least one lipid moiety is conjugated to a nucleotide at position 14, position 15, position 19, position 20, position 23, position 28, position 29 or position 30 of the sense strand, and wherein positions are numbered 5′ to 3′.
Embodiment II-97. A kit comprising the oligonucleotide of any one of Embodiments II-1-68, an optional pharmaceutically acceptable carrier, and a package insert comprising instructions for administration to a subject having a disease, disorder or condition associated with expression of an oligodendrocyte mRNA.
Embodiment II-98. The kit of Embodiment II-97, wherein the package insert comprises instructions for intrathecal administration.
Embodiment II-99. Use of the oligonucleotide of any one of Embodiments II-1-68 or the pharmaceutical composition of Embodiment II-69, in the manufacture of a medicament for the treatment of a disease, disorder or condition associated with expression of an oligodendrocyte mRNA.
Embodiment II-100. The oligonucleotide of any one of Embodiments II-1-68 or the pharmaceutical composition of Embodiment II-69, for use, or adaptable for use, in the treatment of a disease, disorder or condition associated with expression of an oligodendrocyte mRNA.

### EXAMPLES

### Example 1: Preparation of RNAi Oligonucleotides

### Oligonucleotide Synthesis and Purification

The oligonucleotides (RNAi oligonucleotides) described in the Examples were chemically synthesized using methods described herein. Generally, RNAi oligonucleotides were synthesized using solid phase oligonucleotide synthesis methods as described for 19-23mer RNAi oligonucleotides (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g., Hughes and Ellington (2017) COLD SPRING HARB PERSPECT BIOL. 9(1):a023812; Beaucage S. L., Caruthers M. H. STUDIES ON NUCLEOTIDE CHEMISTRY V: Deoxynucleoside Phosphoramidites-A New Class of Key Intermediates for Deoxypolynucleotide Synthesis, TETRAHEDRON LETT. 1981; 22:1859-62. doi: 10.1016/S0040-4039(01)90461-7); PCT application No. PCT/US2021/42469 (each of which is incorporated herein by this reference)).

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84) and the phosphoramidite synthesis as shown below:

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

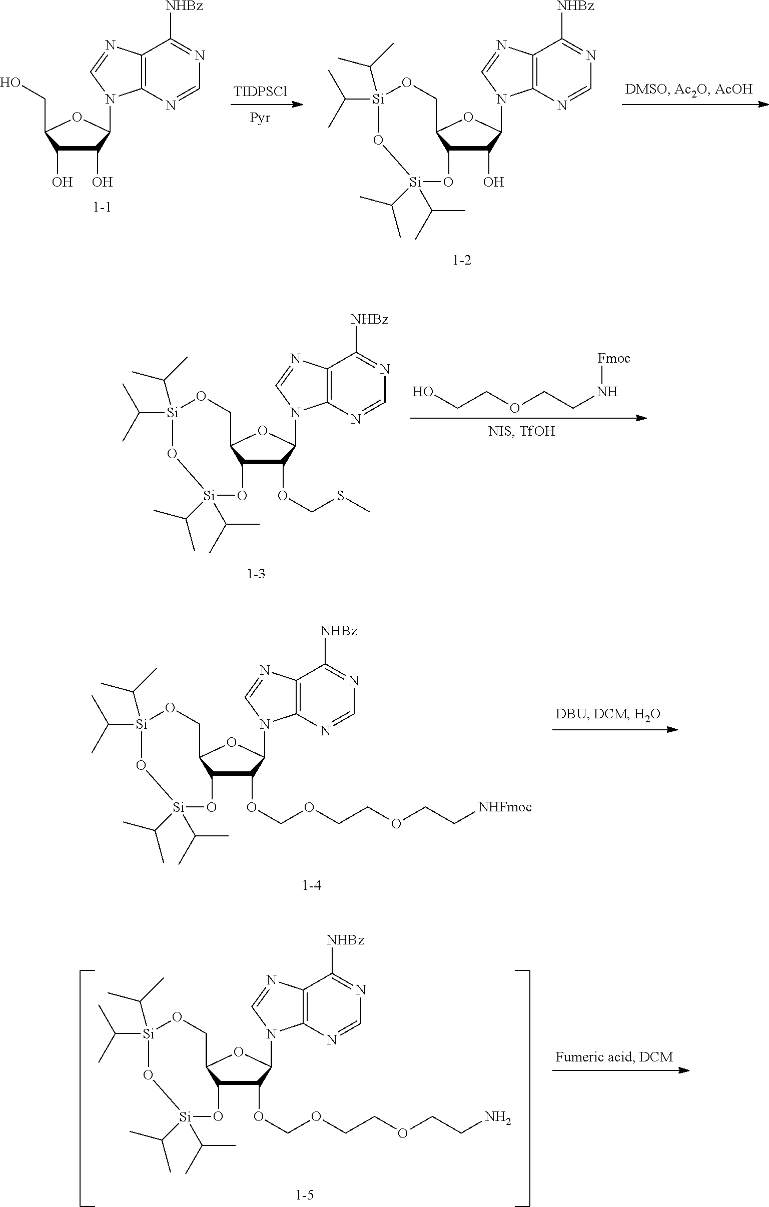

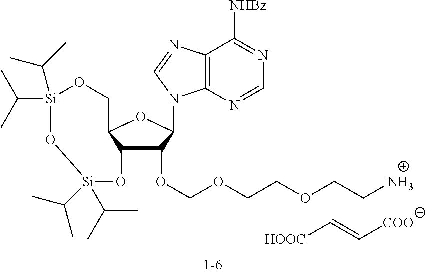

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHZ, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

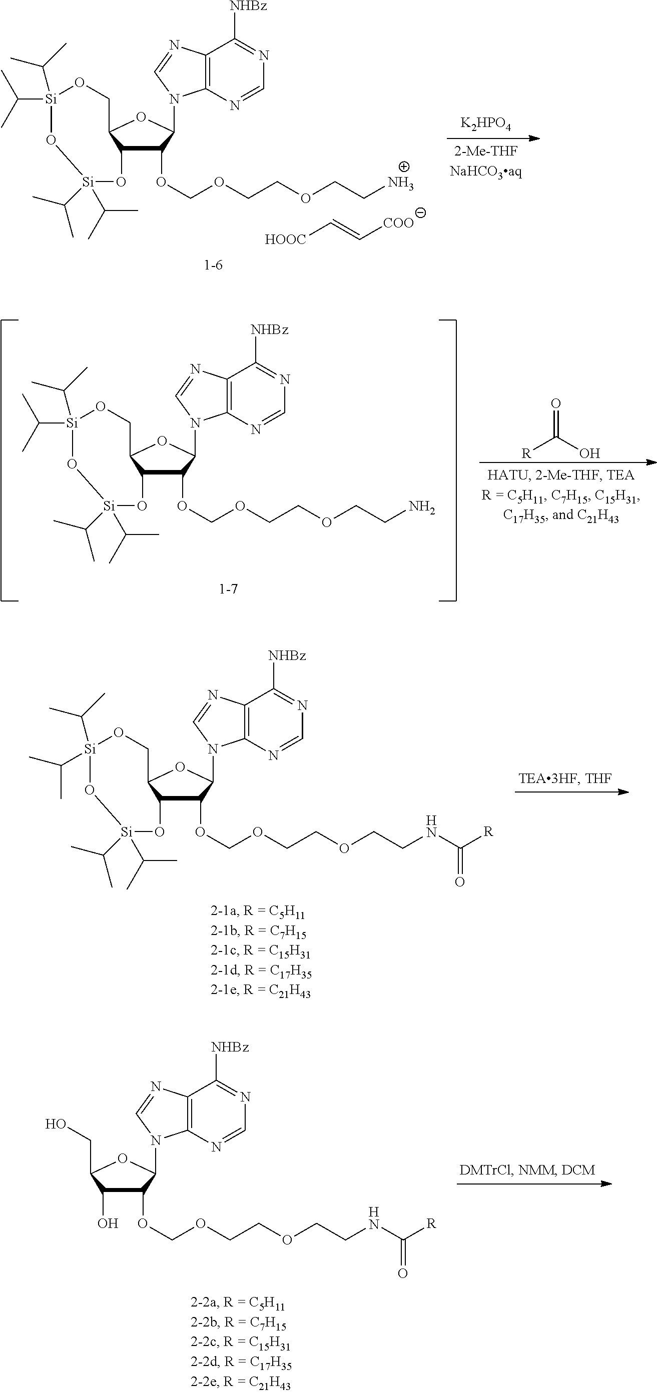

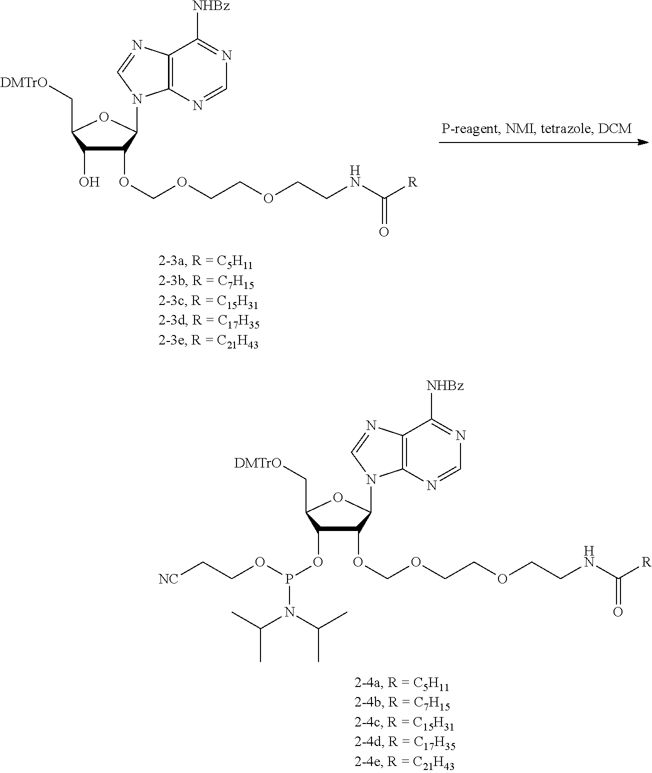

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHZ, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHZ, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHZ, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHZ, d₆-DMSO) 149.41, 149.15.

The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

The synthesis methods described herein are used to generate the lipid-conjugated oligonucleotides described in the Examples below.

### Example 2: RNAi Oligonucleotide-lipid Conjugates Having a Stem-tetraloop at the 5′ Terminus of the Sense Strand Provide Reduced Expression of a Neuronal Target Gene in the CNS

To identify an RNAi oligonucleotide-lipid conjugate capable of reducing mRNA expression in neurons of CNS, a series of C16-conjugated RNAi oligonucleotides were generated by methods described in Example 1. Each oligonucleotide tested comprised an antisense strand having a region of complementarity to mRNA encoding Tubulin Beta 3 Class III (Tubb3). Tubb3 is a protein primarily expressed in neurons and is targeted herein to demonstrate delivery of lipid-conjugated RNAi oligonucleotides to neurons of the CNS.

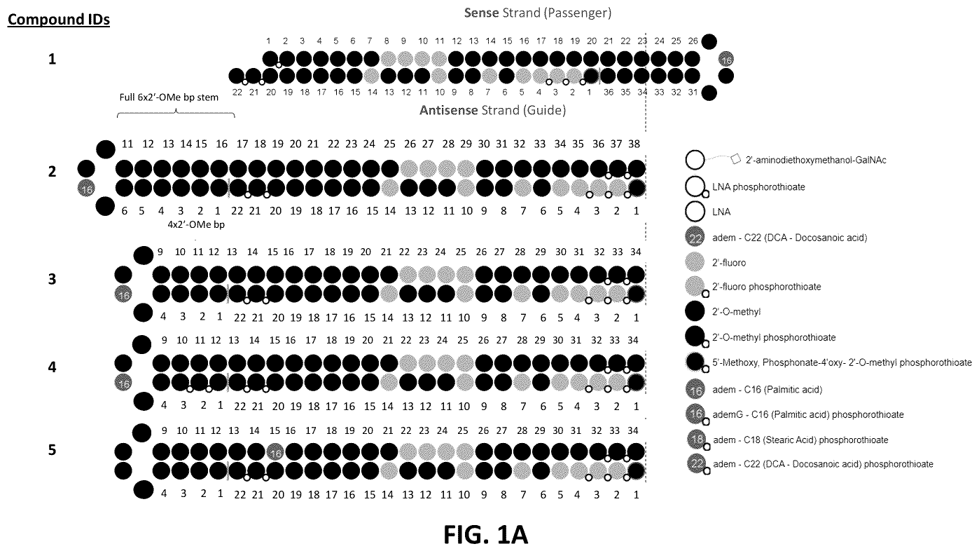

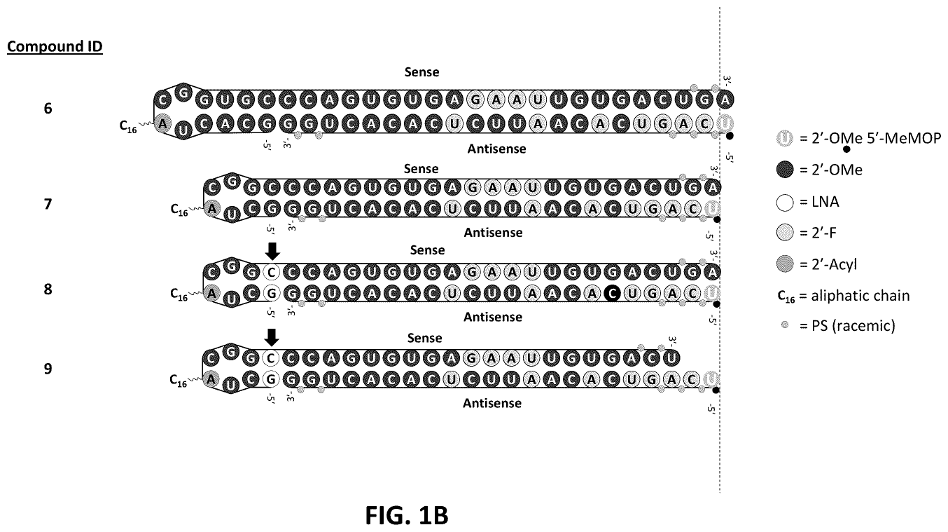

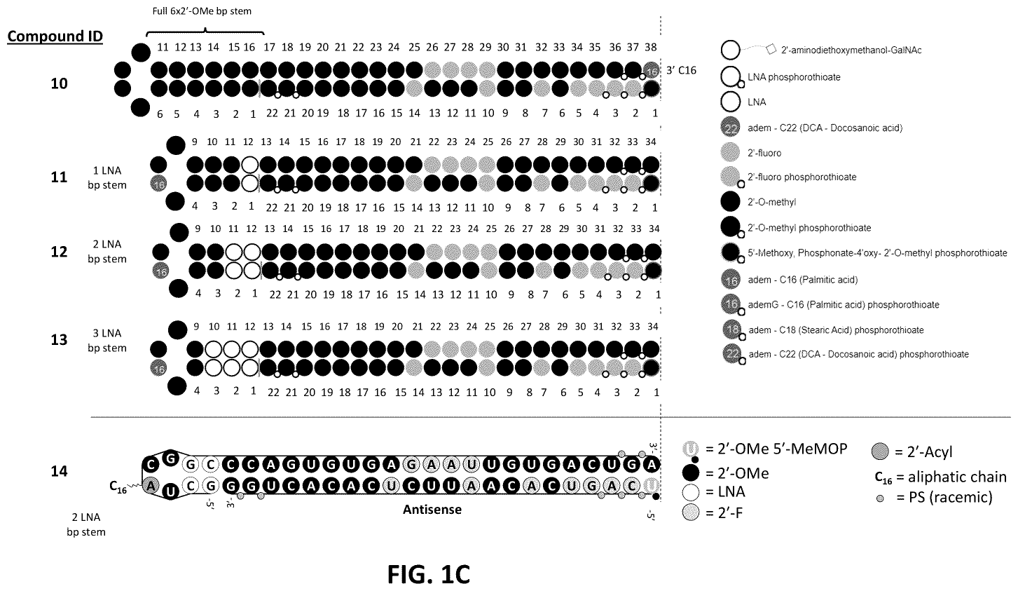

Comparison was made to a parent oligonucleotide-lipid conjugate evaluated in previous studies that had the structure of Compound 1 as shown in FIG. 1A. Compound 1 contains a stem-loop at the 3′ terminus of the sense strand and a 2-nt overhang at the 3′ terminus of the antisense strand. The stem-loop contains a tetraloop having the nucleotide sequence 5′-GAAA-3′, a stem of 6-nt in length, and a C16 lipid conjugated at the second nucleotide of the tetraloop (i.e., the underlined “A” of 5′-GAAA-3′).

RNAi oligonucleotide-lipid conjugates that were evaluated in comparison to Compound 1 had structures according to Compounds 2-14 as shown in FIGS. 1A-1C. These structures contain the structural features described below:
(i) Compounds 2-5 as shown in FIG. 1A have a sense strand with a stem-loop at the 5′ terminus and a blunt-end at the 3′ terminus. The stem-loop has (a) a tetraloop with the sequence 5′-GAAA-3′; and (b) a stem of 6-nt in length (Compound 2) or 4-nt in length (Compounds 3-5). Compounds 2-4 have a C16 lipid positioned at the second nucleotide of the tetraloop (i.e., the underlined “A” of 5′-GAAA-3′) and Compound 5 has a C16 lipid at position 15 of the sense strand. The 5′ terminus of the sense strand for Compound 4 has a series of phosphorothioate (PS) backbone linkages (Compound 4).
(ii) Compounds 6-9 as shown in FIG. 1B contain a sense strand having a stem-loop at the 5′ terminus containing (a) a tetraloop with the sequence 5′-UACG-3′; (b) a stem of 4-nt in length (Compound 6) or 2-nt in length (Compounds 7-9); and (c) a C16 lipid conjugated at the second nucleotide of the tetraloop (i.e., the underlined “A” of the 5′-UACG-3′ tetraloop). Compounds 8 and 9 have a locked nucleic acid (LNA) at the base of the stem. The 3′ terminus of the sense strand has a blunt end (Compounds 6-8) or a 2-nucleotide truncation resulting in a 2-nt overhang at the 5′ terminus of the antisense strand (Compound 9).
(iii) Compound 10 as shown in FIG. 1C contains a sense strand having a stem-loop at the 5′ terminus and a blunt-end at the 3′ terminus. The stem-loop contains (a) a tetraloop with the sequence 5′-GAAA-3′; and (b) a stem of 6-nt in length. The 3′ terminus of the sense strand has a C16 lipid conjugate.
(iv) Compounds 11-13 as shown in FIG. 1C have a sense strand with a stem-loop at the 5′ terminus and a blunt-end at the 3′ terminus. The stem-loop has (a) a tetraloop with the sequence 5′-GAAA-3′; (b) a stem of 4-nt in length with 1-3 LNAs (Compounds 11-13 respectively) in the stem; and (c) a C16 lipid conjugate positioned at the second nucleotide of the tetraloop (i.e., the underlined “A” of 5′-GAAA-3′). The 3′ stem of Compound 13 contains an LNA with a thymine nucleobase rather than uridine nucleobase.
(v) Compound 14 as shown in FIG. 1C has a sense strand with a stem-loop at the 5′ terminus and a blunt-end at the 3′ terminus. The stem-loop has (a) a tetraloop with the sequence 5′-UACG-3′; (b) a stem of 2-nt in length composed of LNAs; and (c) a C16 lipid conjugate positioned at the second nucleotide of the tetraloop (i.e., the underlined “A” of 5′-UACG-3′). The 5′ terminus of the antisense strand was pre-trimmed to expose a 5′OH.

To evaluate the RNAi oligonucleotide-lipid conjugates, C57BL/6 female mice, 6-8 weeks old, were given a single lumbar intrathecal (i.t.) injection with 500 μg of oligonucleotide-lipid conjugate formulated in artificial cerebrospinal fluid (aCSF). The RNAi oligonucleotide-lipid conjugate administered in each treatment group is outlined in Table 1. Group A were control mice that received aCSF.

TABLE 1
Lipid-conjugated RNAi oligonucleotides

| | | | |
|---|---|---|---|
| | | Sense | Antisense |
| Group | Compound | SEQ ID NOs | SEQ ID NOs |

| # | ID | Unmod | Mod | Unmod | Mod |
|---|---|---|---|---|---|
| A | — | — | — | — | — |
| B | 1 | 310 | 338 | 324 | 352 |
| C | 2 | 311 | 339 | 325 | 353 |
| D | 3 | 312 | 340 | 326 | 354 |
| E | 4 | 313 | 341 | 327 | 355 |
| F | 5 | 314 | 342 | 328 | 356 |
| G | 6 | 315 | 343 | 329 | 357 |
| H | 7 | 316 | 344 | 330 | 358 |
| I | 8 | 317 | 345 | 331 | 359 |
| J | 9 | 318 | 346 | 332 | 360 |
| K | 10 | 319 | 347 | 333 | 361 |
| L | 11 | 320 | 348 | 334 | 362 |
| M | 12 | 321 | 349 | 335 | 363 |
| N | 13 | 322 | 350 | 336 | 364 |
| O | 14 | 323 | 351 | 337 | 365 |

Target knockdown was assessed 7 days post-injection. RNA was extracted from the lumbar spinal cord, lumbar dorsal root ganglion (DRG), medulla, hippocampus, and frontal cortex to determine murine Tubb3 mRNA levels by qPCR (normalized to endogenous housekeeping gene Rpl23). The qPCR was performed using PrimeTime™ qPCR Probe Assays. The percentage of murine Tubb3 mRNA remaining in the samples from treated mice was determined using the 2-ΔΔCt (“delta-delta Ct”) method (Livak and Schmittgen (2001) METHODS 25:402-408).

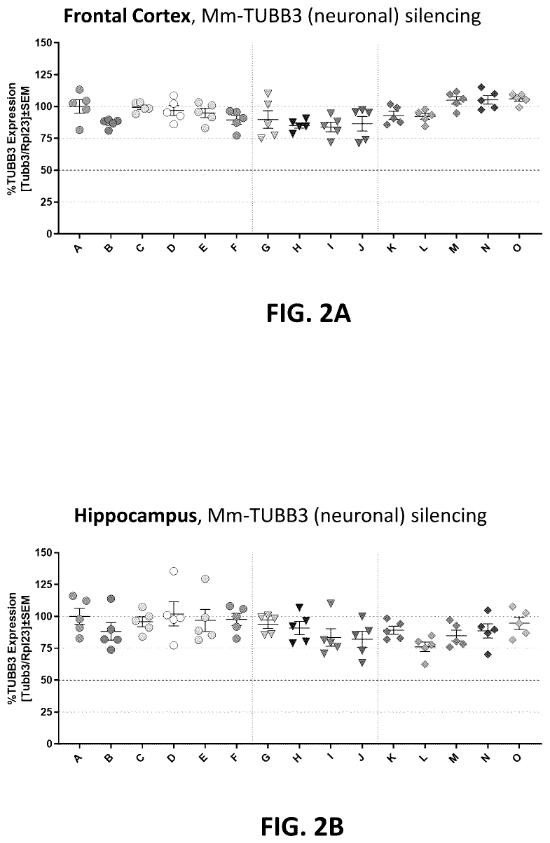

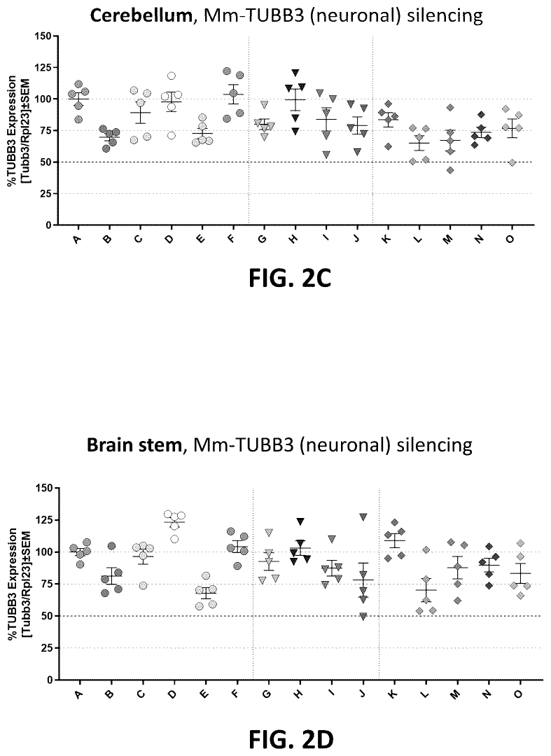

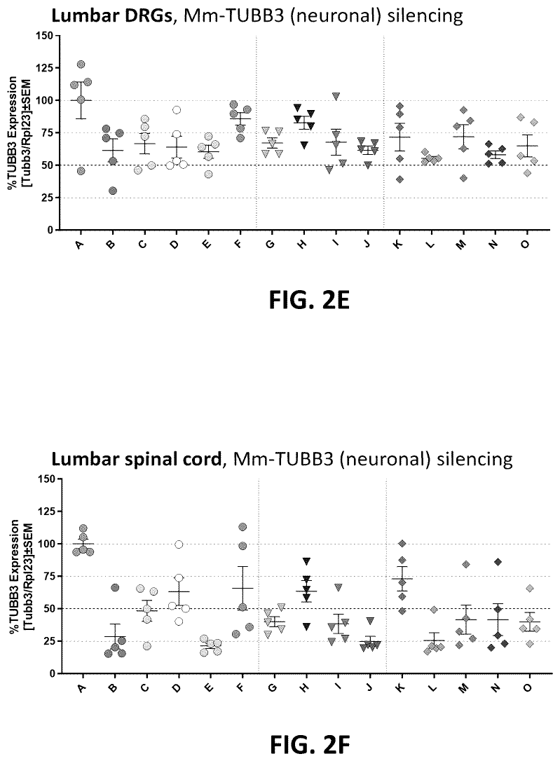

Percent TUBB3 mRNA expression in the frontal cortex and hippocampus is shown in FIGS. 2A-2B respectively. Knockdown was observed in these tissues for most of the treatment groups.

Percent TUBB3 mRNA expression in the cerebellum, brain stem, lumbar DRGs, and lumbar spinal cord is shown in FIGS. 2C-2F respectively. Knockdown was observed in cerebellum and brain stem for most of the treatment groups (FIG. 2C and FIG. 2D). All treatment groups demonstrated silencing of TUBB3 mRNA expression in lumbar DRGs and lumbar spinal cord (FIG. 2E and FIG. 2F). The presence of a stem-loop with high stability at the 5′ terminus of the sense strand increased knockdown of TUBB3 mRNA expression levels in several CNS tissues. Similarly, the presence of an LNA in the stem of the stem-loop structure increased silencing activity.

### Example 3: RNAi Oligonucleotide-Lipid Conjugates Structures with Overhangs in the Antisense Strand Yield Reduced Expression of a Neuronal Target Gene in the CNS

RNAi oligonucleotide-lipid conjugates with the structures of Compound 6-8 that resulted in efficient suppression of TUBB3 expression in CNS tissues as described in Example 2 were compared to TUBB3-targeting oligonucleotides-lipid conjugate structures with additional modifications.

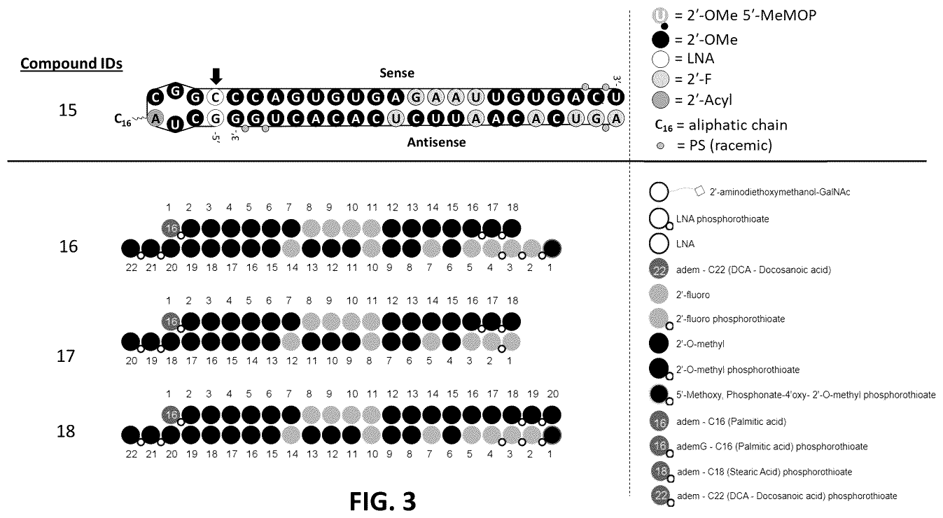

RNAi oligonucleotide-lipid conjugates according to Compounds 7-9 were compared to those having the structures of Compounds 15-17 as shown in FIG. 3 and further described below. Each compounds has an antisense strand with a region of complementarity to TUBB3 mRNA.
(i) Compound 15 has a sense strand with a stem-loop at the 5′ terminus and a blunt-end at the 3′ terminus. The stem-loop contains (a) a tetraloop with the sequence 5′-UACG-3′; (b) a stem of 2-nt in length and having 1 LNA; and (iii) a C16 lipid conjugate at the second nucleotide of the tetraloop (i.e., the underlined “A” of 5′-UACG-3′). The 5′ terminus of the antisense strand was pre-trimmed to expose a 5′OH.
(ii) Compounds 16 has a sense strand with a 2-nt truncation at the 5′ terminus and a 2-nt truncation at the 3′ terminus (i.e., resulting in a 2-nt overhang on the 5′ terminus and 3′ terminus of the antisense strand). A C16 lipid conjugate was positioned at the 5′ terminus of the sense strand.
(iii) Compound 17 has a sense strand with a 2-nt truncation at the 5′ terminus (i.e., resulting in an antisense strand with an overhang at the 3′ terminus), a 3′ terminus with a blunt end, and a C16 lipid conjugate positioned at the 5′ terminus. The 5′ terminus of the antisense strand is truncated to 20-nt in length and pre-trimmed to expose a 5′OH.
(iv) Compound 18 has a sense strand with a 2-nt truncation at the 5′ terminus (i.e., resulting in an antisense strand with an overhang at the 3′ terminus) and a blunt-end at the 3′ terminus. A C16 lipid conjugate is positioned at the 5′ terminus of the sense strand.

To evaluate the RNAi oligonucleotide-lipid conjugates, 6-8 weeks old C57BL/6 female mice were given a single lumbar i.t. injection with 500 μg of oligonucleotide-lipid conjugate formulated in aCSF. The RNAi oligonucleotide-lipid conjugate administered in each animal group are indicated in Table 2. Group A were control mice that received aCSF.

TABLE 2
Lipid-conjugated RNAi oligonucleotide
administered to animal groups A-H

| | | | |
|---|---|---|---|
| | | Sense | Antisense |
| Group | Compound | SEQ ID NOs | SEQ ID NOs |

| # | ID | Unmod | Mod | Unmod | Mod |
|---|---|---|---|---|---|
| A | — | — | — | — | — |
| B | 7 | 316 | 344 | 330 | 358 |
| C | 8 | 317 | 345 | 331 | 359 |
| D | 9 | 318 | 346 | 332 | 360 |
| E | 15 | 369 | 383 | 376 | 390 |
| F | 16 | 370 | 384 | 377 | 391 |
| G | 17 | 371 | 385 | 378 | 392 |
| H | 18 | 372 | 386 | 379 | 393 |

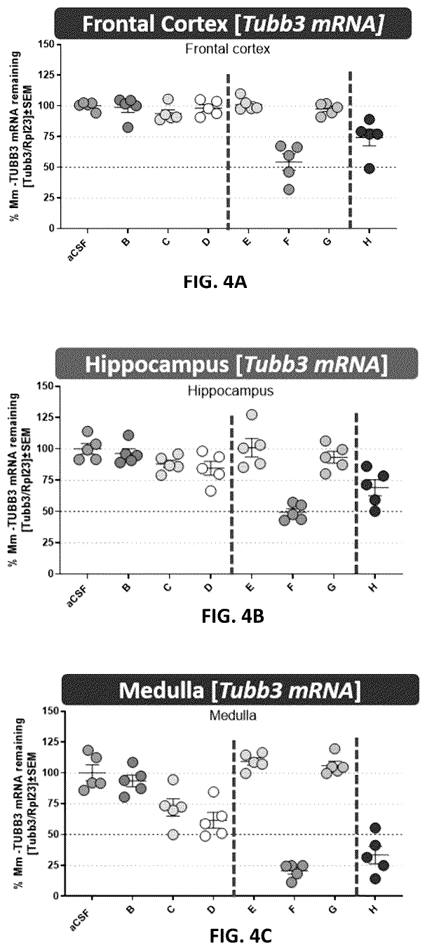

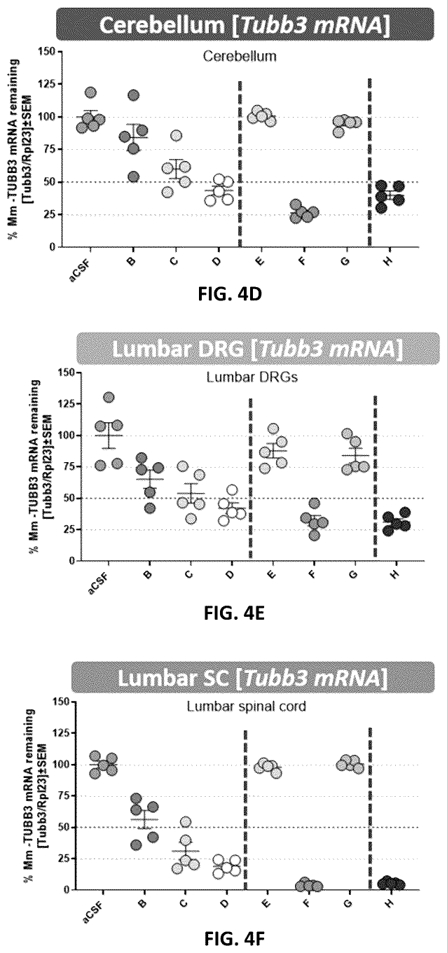

Target knockdown was assessed 7 days post-injection. RNA was extracted from various CNS tissues to determine murine TUBB3 mRNA levels by qPCR (normalized to endogenous housekeeping gene Rpl23) as described in Example 2. Percent TUBB3 mRNA expression in the frontal cortex, hippocampus, medulla, cerebellum, lumbar DRGs, and lumbar spinal cord is shown in FIGS. 4A-4F respectively. Knockdown of TUBB3 mRNA expression was observed in each tissue for all or most of the treatment groups.

### Example 4: RNAi Oligonucleotide-Lipid Conjugates Having a Truncation at the 3′ Terminus of the Sense Strand Provide Enhanced Activity for Reducing Expression of a Neuronal Target Gene in the CNS

RNAi oligonucleotide-lipid conjugates designed to target TUBB3 mRNA and having a 2-nt overhang at the 3′ terminus of the antisense strand were prepared to evaluate the effect of truncations at the 3′ terminus of the sense strand on TUBB3 mRNA expression in the CNS.

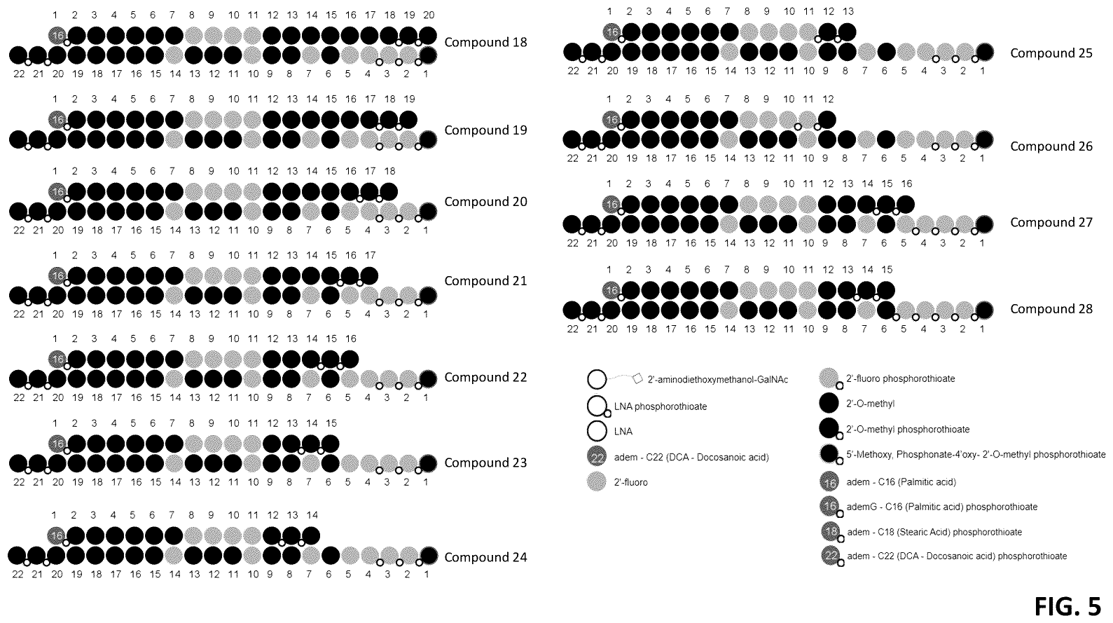

Comparison was made to a parent RNAi oligonucleotide-lipid conjugate having the structure of Compound 18 as shown in FIG. 5. Compound 18 has an antisense strand with a 2-nt overhang at the 3′ terminus and a sense strand with a blunt end at the 3′ terminus of the sense strand and a C16 lipid conjugate at the 5′ terminus.

The RNAi oligonucleotide-lipid conjugates compared to Compound 18 had structures according to Compounds 19-28 as shown in FIG. 5.

Each of Compounds 19-26 has an antisense strand with a 2-nt overhang at the 3′ terminus and a sense strand with a C16 lipid conjugate at the 5′ terminus. Compounds 19-26 further contain an overhang at the 5′ terminus of the antisense strand that is 1-nt to 8-nt respectively. Compounds 27 and 28 respectively contain a 4-nt or 5-nt overhang at the 5′ terminus of the antisense strand with nucleotides at position 2-5 of the exposed overhang having PS-backbone linkages.

To evaluate the RNAi oligonucleotide-lipid conjugates, C57BL/6 female mice were given a single lumbar i.t. injection with 500 μg of oligonucleotide-lipid conjugate formulated in aCSF. The RNAi oligonucleotide-lipid conjugate administered in each animal group are indicated in Table 3. Group A were control mice that received aCSF.

| | | | |
|---|---|---|---|
| | | Sense | Antisense |
| Group | Compound | SEQ ID NOs | SEQ ID NOs |

| # | ID | Unmod | Mod | Unmod | Mod |
|---|---|---|---|---|---|
| A | — | — | — | — | — |
| B | 18 | 372 | 386 | 379 | 393 |
| C | 19 | 395 | 417 | 406 | 428 |
| D | 20 | 370 | 384 | 377 | 391 |
| E | 21 | 397 | 419 | 408 | 430 |
| F | 22 | 398 | 420 | 409 | 431 |
| G | 23 | 399 | 421 | 410 | 432 |
| H | 24 | 400 | 422 | 411 | 433 |
| I | 25 | 401 | 423 | 412 | 434 |
| J | 26 | 402 | 424 | 413 | 435 |
| K | 27 | 403 | 425 | 414 | 436 |
| L | 28 | 404 | 426 | 415 | 437 |

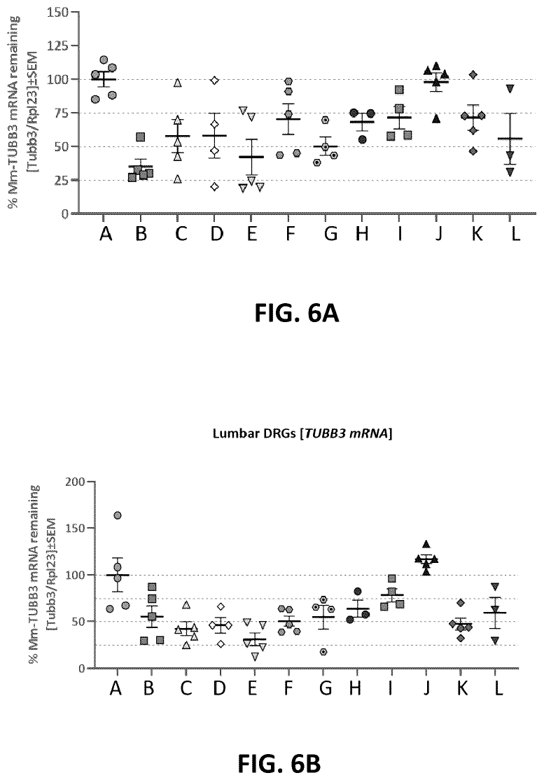

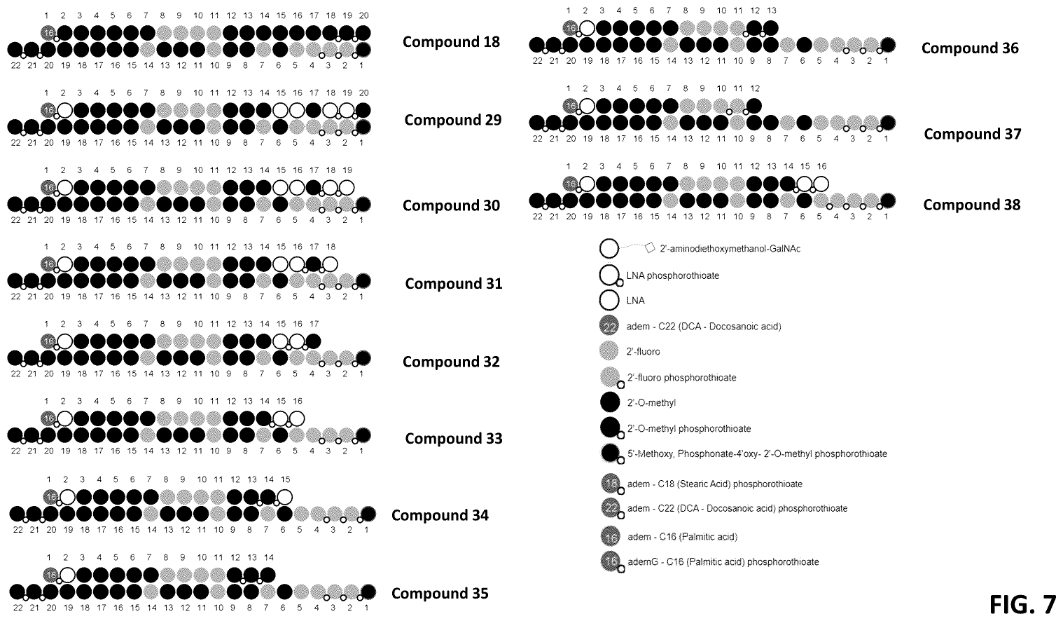

Target knockdown was assessed 7 days post-injection. RNA was extracted from various CNS tissues to determine murine TUBB3 mRNA levels by qPCR (normalized to endogenous housekeeping gene Rpl23) as described in Example 2. Percent TUBB3 mRNA expression in the cerebellum and lumbar DRGs are shown in FIGS. 6A-6B respectively.

It was observed that a truncation of up to 3-nt at the 3′ terminus of the sense strand was tolerated without diminishing TUBB3 mRNA suppression compared to Compound 18. See 3′p-3; 3′p-2; and 3′p-1 vs parent in FIGS. 6A-6B. In contrast, truncations of 4-nt or more at the 3′ terminus of the sense strand generally reduced, but did not abolish, reduction of target mRNA expression. See groups F-J vs B in FIGS. 6A-6B.

The effect of truncations at the 3′ terminus of the sense strand in combination with LNAs positioned within the senses strand was evaluated. The RNAi oligonucleotide-lipid conjugates that were evaluated had structures according to Compounds 29-38 as shown in FIG. 7.

Compound 29 has a structure equivalent to Compound 18, but includes an LNA at positions 2, 15, 16, 18, and 19 of the sense strand. Compounds 30-37 further include truncations at the 3′ terminus of the sense strand that are respectively 1-nt to 8-nt in length. Compound 38 contains a 4-nt truncation at the 3′ terminus of the sense strand and nucleotides at position 2-5 of the exposed overhang of the antisense containing a PS backbone linkage.

To evaluate the RNAi oligonucleotide-lipid conjugates, C57BL/6 female mice were given a single lumbar i.t. injection with 500 μg of oligonucleotide-lipid conjugate formulated in aCSF. The RNAi oligonucleotide-lipid conjugate administered in each animal group are indicated in Table 4. Group A were control mice that received aCSF.

TABLE 4
RNAi oligonucleotide-lipid conjugates
administered to animal groups A-L

| | | | |
|---|---|---|---|
| | | Sense | Antisense |
| Group | Compound | SEQ ID NOs | SEQ ID NOs |

| # | ID | Unmod | Mod | Unmod | Mod. |
|---|---|---|---|---|---|
| A | — | — | — | — | — |
| B | 18 | 372 | 386 | 379 | 393 |
| C | 29 | 439 | 461 | 450 | 472 |
| D | 30 | 440 | 462 | 451 | 473 |
| E | 31 | 441 | 463 | 452 | 474 |
| F | 32 | 442 | 464 | 453 | 475 |
| G | 33 | 443 | 465 | 454 | 476 |
| H | 34 | 444 | 466 | 455 | 477 |
| I | 35 | 445 | 467 | 456 | 478 |
| J | 36 | 446 | 468 | 457 | 479 |
| K | 37 | 447 | 469 | 458 | 480 |
| L | 38 | 448 | 470 | 489 | 481 |

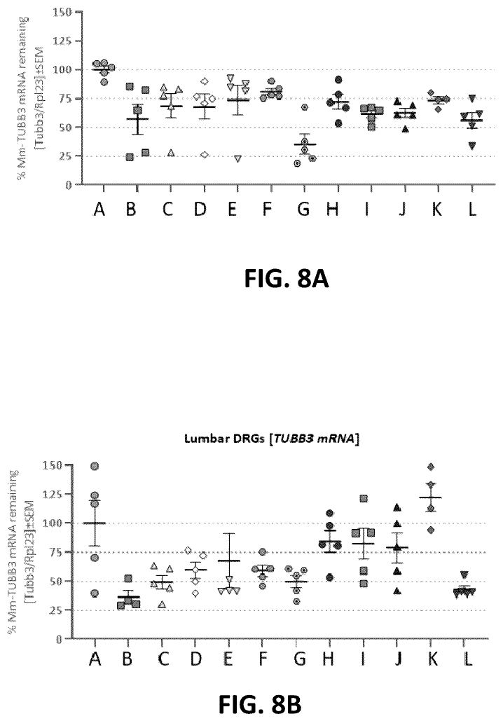

Target knockdown was assessed 7 days post-injection. RNA was extracted from various CNS tissues to determine murine TUBB3 mRNA levels by qPCR (normalized to endogenous housekeeping gene Rpl23) as described in Example 2. Percent TUBB3 mRNA expression in the cerebellum and lumbar DRGs is shown in FIGS. 8A-8B respectively. Knockdown of TUBB3 mRNA expression was observed in both tissues for all or most of the treatment groups.

### Example 5: RNAi Oligonucleotide-Lipid Conjugates Evaluated for Reduction of Target Gene Expression in Ocular Tissue

To identify an RNAi oligonucleotide-lipid conjugate capable of reducing mRNA expression ocular tissues, a series of lipid-conjugated RNAi oligonucleotides were generated by methods described in Example 1. Each oligonucleotide tested had an antisense strand with a region of complementarity to mRNA encoding rat reticulon 4 (RTN4), which is a developmental neurite growth regulatory factor expressed in the eye.

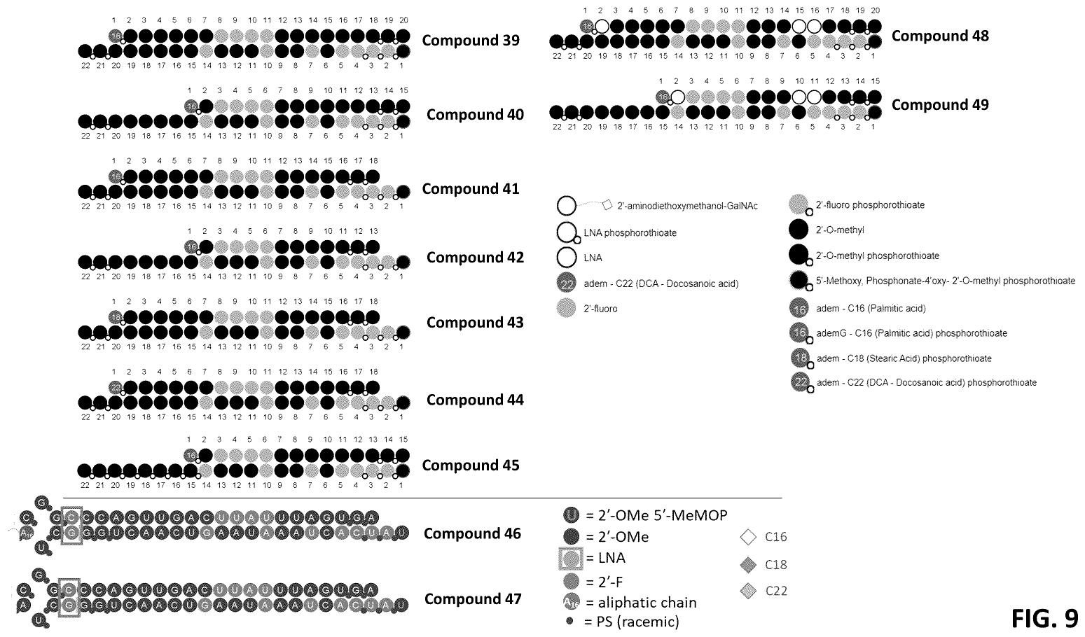

Comparison was made to a parent RNAi oligonucleotide-lipid conjugate having a structure according to Compound 39 shown in FIG. 9. Compound 39 has an antisense strand with a 2-nt overhang at the 3-terminus and a blunt-end at the 5′ terminus. Compound 39 further contains a C16 lipid conjugate at the 5′ terminus of the sense strand.

The RNAi oligonucleotide-lipid conjugates evaluated had variation in the length of the aliphatic lipid, inclusion of LNAs, and truncation at the 5′ and/or 3′ termini of the sense strand. The structures of the oligonucleotide-lipid conjugates are shown in FIG. 9 and further detailed below:
(i) Compound 40 contains a sense strand having a 5-nt truncation at the 5′ terminus, a blunt-end at the 3′ terminus, and a C16 lipid conjugate at position 1 of the sense strand;
(ii) Compound 41 contains a sense strand having a 2-nt truncation at the 5′ terminus, a 2-nt truncation at the 3′ terminus, and a C16 lipid conjugate at position 1 of the sense strand;
(iii) Compound 42 contains a sense strand having a 5-nt truncation at the 5′ terminus, a 2-nt truncation at the 3′ terminus, and a C16 lipid conjugate at position 1 of the sense strand;
(iv) Compound 43 contains a sense strand having a 2-nt truncation at the 5′ terminus, a 2-nt truncation at the 3′ terminus, and a C18 lipid conjugate at position 1 of the sense strand;
(v) Compound 44 contains a sense strand having a 2-nt truncation at the 5′ terminus, a 2-nt truncation at the 3′ terminus, and a C22 lipid conjugate at position 1 of the sense strand;
(vi) Compound 45 contains a sense strand having a 5-nt truncation at the 5′ terminus, a blunt end at the 3′ terminus, and a C16 lipid conjugate at position 1 of the sense strand. The exposed overhang of the antisense strand contained PS linkages between neighboring residues;
(vii) Compounds 46-47 each contains a sense strand having a stem-loop at the 5′ terminus and a 2-nt truncation at the 3′ terminus. The stem-loop has (i) a tetraloop with the sequence 5′-UACG-3′; and (ii) a stem of 2 nt in length composed of LNAs. Compound 46 contains a C16 lipid conjugate at position 4 of the sense strand (i.e., the underlined “A” of the 5′-UACG-3′ tetraloop), whereas Compound 47 does not contain a lipid conjugate.
(viii) Compound 48 contains a sense strand having a 2-nt truncation at the 5′ terminus, a blunt end at the 3′ terminus, a C16 lipid conjugate at position 1, and LNAs at positions 2, 15, and 16; and
(ix) Compound 49 contains a sense strand having a 5-nt truncation at the 5′ terminus, a blunt end at the 3′ terminus, a C16 lipid conjugate at position 1, and LNAs at positions 2, 10, and 11.

To evaluate the RNAi oligonucleotide-lipid conjugates, Sprague-Dawley rats were given an intravitreal injection per eye, each with 250 μg of oligonucleotide-lipid conjugate. The RNAi oligonucleotide-lipid conjugate administered in each animal group is outlined in Table 5. Group A were control rats that received PBS.

TABLE 5
Lipid-conjugated RNAi oligonucleotides
administered to animal groups A-L

| | | | |
|---|---|---|---|
| | | Sense | Antisense |
| Group | Compound | SEQ ID NOs | SEQ ID NOs |

| # | ID | Unmod | Mod | Unmod | Mod |
|---|---|---|---|---|---|
| A | — | — | — | — | — |
| B | 39 | 482 | 504 | 493 | 515 |
| C | 40 | 483 | 505 | 494 | 516 |
| D | 41 | 484 | 506 | 495 | 517 |
| E | 42 | 485 | 507 | 496 | 518 |
| F | 43 | 486 | 508 | 497 | 519 |
| G | 44 | 487 | 509 | 498 | 520 |
| H | 45 | 488 | 510 | 499 | 521 |
| I | 46 | 489 | 511 | 500 | 522 |
| J | 47 | 490 | 512 | 501 | 523 |
| K | 48 | 491 | 513 | 502 | 524 |
| L | 49 | 492 | 514 | 503 | 525 |

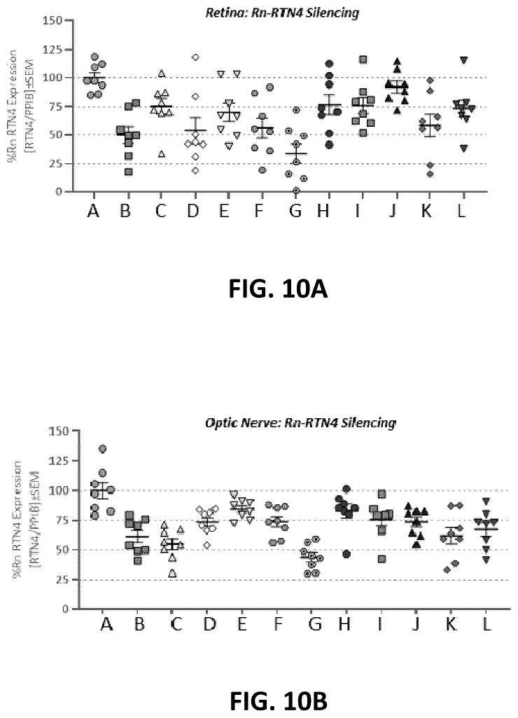

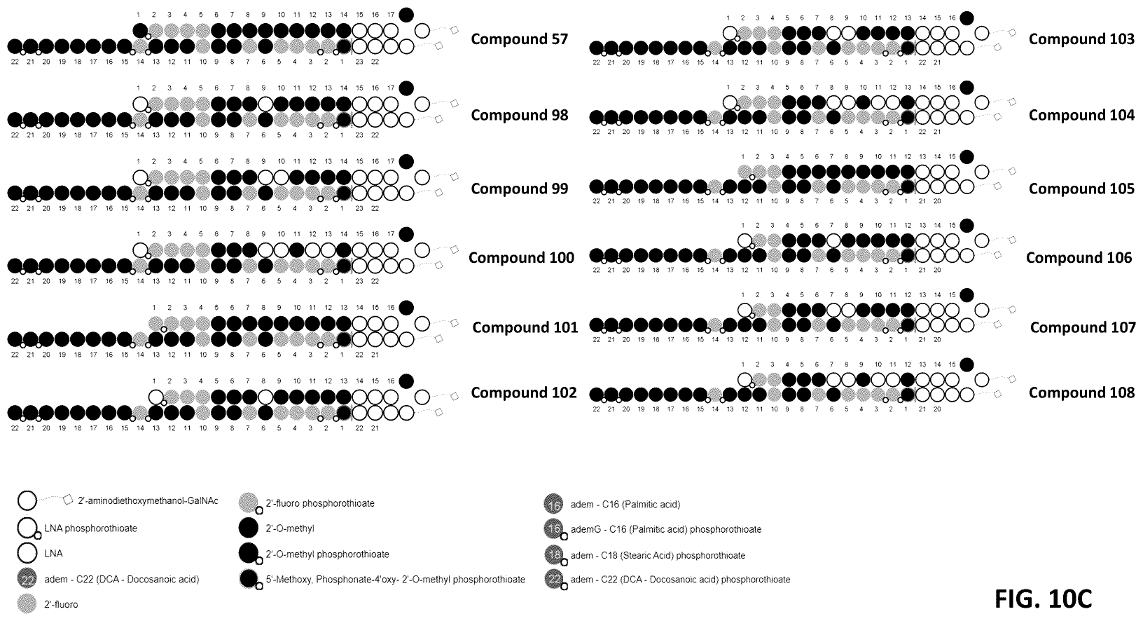

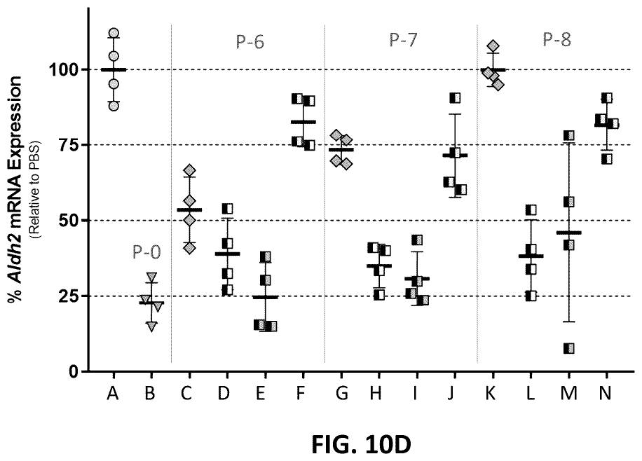

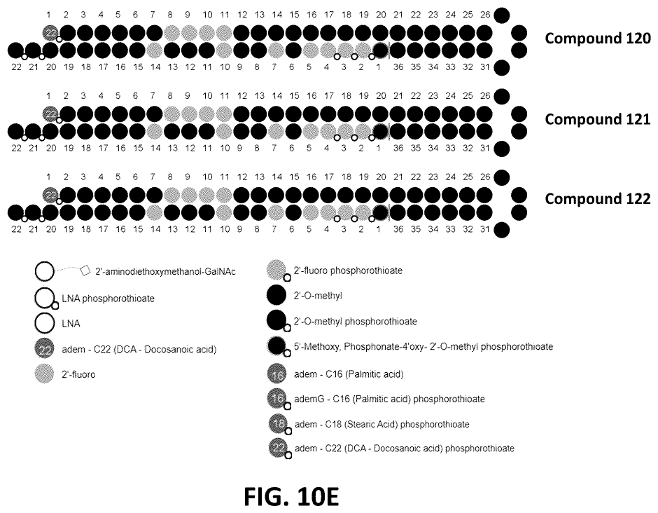

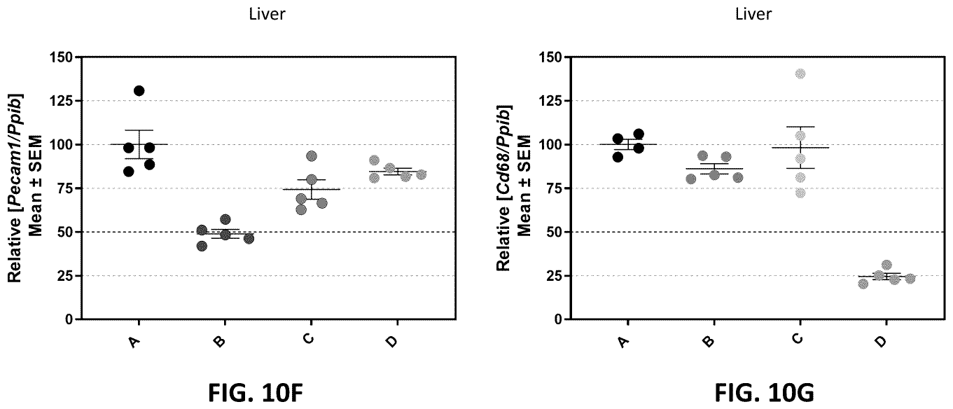

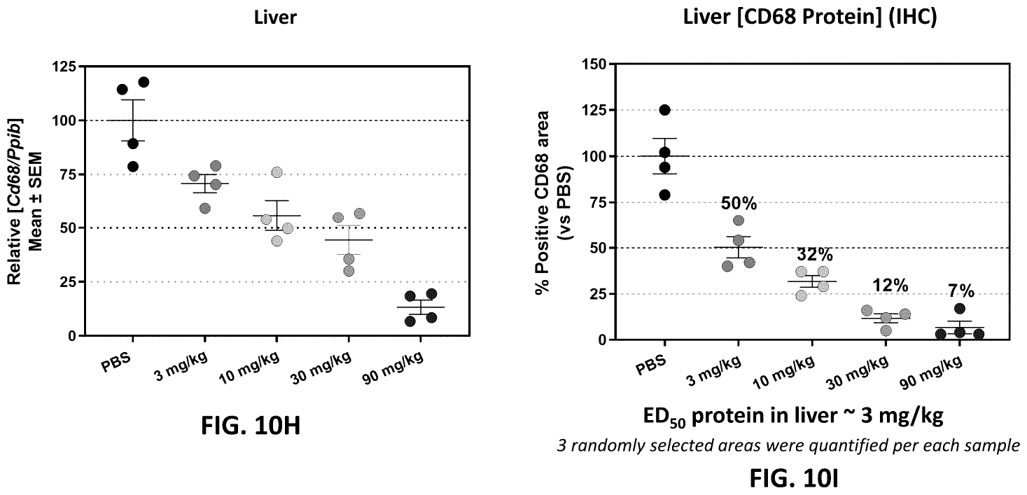

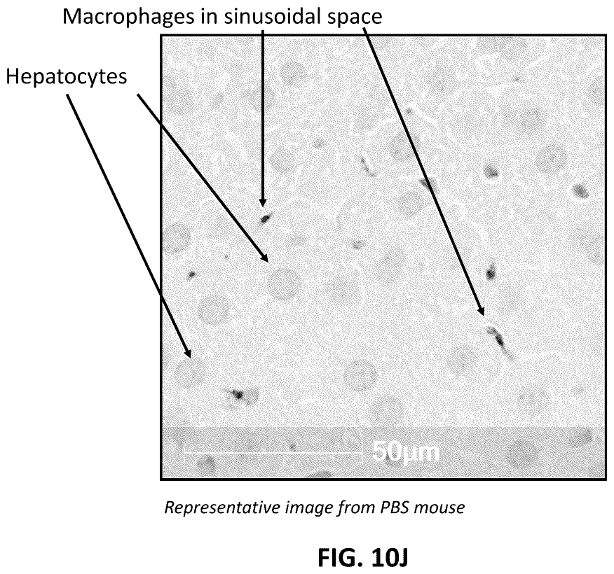

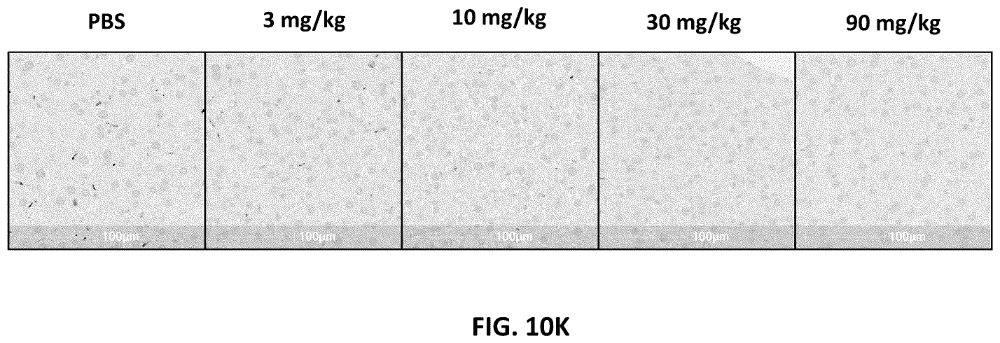

Target knockdown was assessed 14 days post-injection. RNA was extracted from retina and optic nerve of one eye to determine rat RTN4 mRNA levels by qPCR (normalized to endogenous housekeeping gene PPIB). The qPCR was performed using PrimeTime™ qPCR Probe Assays, which consisted of a primer pair and fluorescently labeled 5′ nuclease probe specific to rat RTN4 mRNA. The percentage of rat RTN4 mRNA remaining in the samples from treated rats was determined using the 2-ΔΔCt (“delta-delta Ct”) method (Livak and Schmittgen (2001) METHODS 25:402-408). Percent RTN4 mRNA expression in the is shown in FIGS. 10A-10B respectively. Suppression of RTN4 mRNA expression in both retina and optic nerve tissues was observed for each of the treatment groups.

### Example 6: RNAi Oligonucleotide-GalNAc Conjugates Having Sense Strand Truncations and LNAs Evaluated for Target Knockdown in the Liver

RNAi oligonucleotide-GalNAc conjugates were prepared with a sense strand having a stem-tetraloop at the 3′ terminus, truncations at the 5′ terminus, and inclusion of LNAs on silencing of a target gene in the liver. Each oligonucleotide-lipid conjugate tested comprised an antisense strand having a region of complementarity to mRNA encoding Aldh2.

Comparison was made to a parent oligonucleotide-GalNAc conjugate having a sense strand with (a) no truncation at the 5′ terminus, thereby providing a 2-nt overhang at the 3′ terminus of the antisense strand; and (b) a nicked stem-loop at the 3′ terminus. The stem was 3 bp in length and composed of LNAs. The loop contained the sequence 5′-GAA-3′, with the a 2′-aminodiethoxymethanol-GalNAc at position 2 and 3 from the 5′ terminus (i.e., the underlined nucleotides of the 5′-GAA-3′ loop).

Oligonucleotide-GalNAc conjugates that were evaluated included those having the structures of Compounds 57 and 98-108 as shown in FIG. 10C and further described below:
(i) Compounds 57 and 98-100 contain a sense strand having (a) a 6-nt truncation at the 5′ terminus, thereby providing an 8-nt overhang at the 3′ terminus of the antisense strand; and (ii) a nicked stem-loop at the 3′ terminus identical to that of the parent oligonucleotide-GalNAc conjugate. Compound 98 contained an additional LNA at the 5′ terminus of the sense strand; Compound 99 contained two additional LNAs, one at the 5′ terminus and one 9-nt from the 5′ terminus of the sense strand; Compound 100 contained three additional LNAs, one at the 5′ terminus, and two positioned respectively 9-nt and 10-nt from the 5′ terminus of the sense strand; and Compound 101 contained five additional LNAs, one at the 5′ terminus, and four positioned respectively 9-nt, 10-nt, 12-nt, and 13-nt from the 5′ terminus of the sense strand.
(ii) Compounds 101-104 contain a sense strand having (a) a 7-nt truncation at the 5′ terminus, thereby providing an 9-nt overhang at the 3′ terminus of the antisense strand; and (ii) a nicked stem-loop at the 3′ terminus identical to that of the parent oligonucleotide-GalNAc conjugate. Compound 102 contained two additional LNAs, one at the 5′ terminus and one 8-nt from the 5′ terminus of the sense strand; Compound 103 contained three additional LNAs, one at the 5′ terminus, and two positioned respectively 8-nt and 9-nt from the 5′ terminus of the sense strand; and Compound 104 contained five additional LNAs, one at the 5′ terminus, and four positioned respectively 8-nt, 9-nt, 11-nt, and 12-nt from the 5′ terminus of the sense strand.
(ii) Compounds 105-108 contain a sense strand having (a) an 8-nt truncation at the 5′ terminus, thereby providing a 10-nt overhang at the 3′ terminus of the antisense strand; and (ii) a nicked stem-loop at the 3′ terminus identical to that of the parent oligonucleotide-GalNAc conjugate. Compound 106 contained two additional LNAs, one at the 5′ terminus and one 7-nt from the 5′ terminus of the sense strand; Compound 107 contained three additional LNAs, one at the 5′ terminus, and two positioned respectively 7-nt and 8-nt from the 5′ terminus of the sense strand; and Compound 108 contained five additional LNAs, one at the 5′ terminus, and four positioned respectively 7-nt, 8-nt, 10-nt, and 11-nt from the 5′ terminus of the sense strand.

To evaluate the RNAi oligonucleotide-GalNAc conjugates, mice were given a single intravenous (i.v.) injection of 0.5 mg/kg oligonucleotide-GalNAc conjugate formulated in PBS. The RNAi oligonucleotide-GalNAc conjugates administered for each of the treatment groups are outlined in Table 6. Group A were control mice that received PBS only.

TABLE 6
RNAi oligonucleotides-GalNAc conjugates
administered to animal groups A-N

| | | | |
|---|---|---|---|
| | | Sense | Antisense |
| Group | Compound | SEQ ID NOs | SEQ ID NOs |

| # | ID | Unmod | Mod | Unmod | Mod |
|---|---|---|---|---|---|
| A | — | — | — | — | — |
| B | parent | | | | |
| C | 57 | 193 | 217 | 205 | 229 |
| D | 98 | 194 | 218 | 206 | 230 |
| E | 99 | 195 | 219 | 207 | 231 |
| F | 100 | 196 | 220 | 208 | 232 |
| G | 101 | 197 | 221 | 209 | 233 |
| H | 102 | 198 | 222 | 210 | 234 |
| I | 103 | 199 | 223 | 211 | 235 |
| J | 104 | 200 | 224 | 212 | 236 |
| K | 105 | 201 | 225 | 213 | 237 |
| L | 106 | 202 | 226 | 214 | 238 |
| M | 107 | 203 | 227 | 215 | 239 |
| N | 108 | 204 | 228 | 216 | 240 |

Target knockdown was assessed 4 days post-injection. RNA was extracted from liver tissue and murine ALDH2 mRNA levels were determined by qPCR. Reduction of target mRNA was measured by qPCR using CFX384 TOUCH™ Real-Time PCR Detection System (BioRad Laboratories, Inc., Hercules, CA). All samples were normalized to the PBS treated control animals and plotted using GraphPad Prism software (GraphPad Software Inc., La Jolla, CA). Normalized ALDH2 mRNA expression in the liver relative to control as measured for each treatment group is shown in FIG. 10D. Suppression of ALDH2 mRNA expression was observed for most treatment groups.

### Example 7: RNAi Oligonucleotide-Lipid Conjugates for Reducing Expression of a Marker Highly Expressed by Non-Hepatocytes in the Liver

The liver has a cellular composition that is approximately 60% hepatocytes and 35% non-parenchymal cells (NPCs). The NPCs are composed of macrophages (also referred to as Kupffer cells), liver sinusoidal endothelial cells (LSECs), hepatic stellate cells, and other leukocytes. Macrophages in the liver function to maintain tissue homeostasis and respond to tissue injury (see Braet et al., Seminars in Cell Developmental Biology, 2017). It was evaluated if systemic delivery of RNAi oligonucleotide-lipid conjugates directed to a target gene highly expressed by liver NPC would induce silencing of the target gene in the liver. The target genes selected for evaluation included PECAM1 and CD68, which are highly expressed by NPCs of the liver (specifically LSECs and macrophages respectively) (see Ouyang et al (2014) Oncology Lett; Klein, et al (2021) Mol. Ther).

The RNAi oligonucleotide lipid conjugates evaluated had the structure of Compounds 120-122 as shown in FIG. 10E. Each contains a nicked stem-tetraloop at the 3′ terminus of the sense strand and a 2-nucleotide overhang at the 3′ terminus of the antisense strand. The stem-tetraloop contained a tetraloop having the nucleotide sequence 5′-GAAA-3′ and a stem of 6 nt in length. The 5′ terminus of the sense strand contains a C22 lipid conjugate. Compound 120 has an antisense strand having a region of complementarity to a first target sequence in an mRNA encoding PECAM1 (PECAM1-2392); Compound 121 has an antisense strand having a region of complementarity to a second target sequence in an mRNA encoding (PECAM1-3222); and Compound 122 has an antisense strand having a region of complementarity to a target sequence in an mRNA encoding CD68 (CD68-0815).

To evaluate the RNAi oligonucleotide-lipid conjugates targeting PECAM1 and CD68, female CD-1 mice were given a single subcutaneous injection of 30 mg/kg of oligonucleotide-lipid conjugate formulated in PBS. The RNAi oligonucleotide-lipid conjugates administered for each of the treatment groups are outlined in Table 7. Group A were control mice that received PBS only.

TABLE 7
RNAi oligonucleotides-GalNAc conjugates
administered to animal groups A-D

| Group | Compound | Sense SEQ ID NOS | | Antisense SEQ ID NOS | |
|---|---|---|---|---|---|
| # | ID | Unmod | Mod | Unmod | Mod |
| A | — | — | — | — | — |
| B | 120 | 1 | 7 | 4 | 10 |
| C | 121 | 2 | 8 | 5 | 11 |
| D | 122 | 3 | 9 | 6 | 12 |

Target knockdown was assessed 7 days post-injection. RNA was extracted from liver and extra-hepatic tissues and murine PECAM and CD68 mRNA levels were determined by qPCR (normalized to endogenous housekeeping gene Ppib). The percentage of murine mRNA remaining in the samples from treated mice was determined using the 2-ΔΔCt (“delta-delta Ct”) method (Livak and Schmittgen (2001) METHODS 25:402-408).

Percent PECAM1 and CD68 mRNA expression in the liver as measured for each treatment group is respectively shown in FIGS. 10F-10G. Reduction in expression of mRNA encoding PECAM1 was approximately 50% reduction for mice receiving Compound 120. Reduction in expression of mRNA encoding CD68 was approximately 75% for mice receiving Compound 122.

### Example 8: Effect of Dose on Silencing Mediated by an RNAi Oligonucleotide-Lipid Conjugate Targeting a Marker Abundantly Expressed by Liver Macrophages

The dose of the CD68-targeting RNAi oligonucleotide-lipid conjugate described in Example 7 was titrated to determine the effect on silencing of CD68 expression in liver macrophages. Specifically, the RNAi oligonucleotide-lipid conjugate evaluated had the structure of Compound 122 as shown in FIG. 10E.

The oligonucleotide-lipid conjugate was administered to female CD-1 mice as a single subcutaneous injection of 3-90 mg/kg of oligonucleotide-lipid conjugate formulated in PBS, as outlined in Table 8. Group A were control mice that received PBS only.

TABLE 8
Animal groups receiving an CD68-targeting RNAi
oligonucleotide-liver conjugate

| Group | Compound | Dose (mg/kg) |
|---|---|---|
| A | — | 0 |
| B | 122 | 3 |
| C | 122 | 10 |
| D | 122 | 30 |
| E | 122 | 90 |

Target knockdown was assessed 7 days post-injection. RNA was extracted from liver and extra-hepatic tissues and murine CD68 mRNA levels were determined by qPCR as described in Example 7. Liver was also collected for immunohistochemistry (IHC) analysis of CD68 protein levels. The liver was fixed in formalin and staining was performed using an CD68-specific primary antibody. (see, e.g., Li, et al (1996) Mod Pathol 9:982; Kunisch et al (2003) Annu Rheum Disc 63:774; Caffo et al (2005) Neurosurgery 57:551). CD68 expression was quantified for three randomly selected areas of the tissue and provided as an average.

As shown in FIG. 10H, a dose-dependent decrease in expression of CD68 mRNA was observed in liver tissue of mice administered Compound 122. An ED₅₀ of approximately 10 mg/kg was measured and a reduction of approximately 85% in CD68 mRNA expression was observed at the maximum dose (90 mg/kg).

As shown in FIG. 10I, a dose-dependent decrease in CD68 protein expression was also observed in liver tissue of mice administered Compound 122 as measured by IHC. A representative IHC image is shown in FIG. 10J, which demonstrates CD68 protein expression is abundant in liver macrophages. As shown in FIG. 10K, CD68 protein expression was diminished in liver with increased dosing of Compound 122. An ED₅₀ of approximately 3 mg/kg was measured and a reduction of approximately 93% in CD68 protein expression was observed at the maximum dose (90 mg/kg).

### Example 9: RNAi Oligonucleotide-lipid Conjugates for Reducing a Marker Highly Expressed by Macrophages in the Liver

RNAi oligonucleotide-lipid conjugates were evaluated to determine the effect of truncations at the 5′ and/or 3′ terminus of the sense strand on activity for silencing a target gene expressed by liver macrophages. Each oligonucleotide-lipid conjugate tested comprised an antisense strand having a region of complementarity to mRNA encoding CD68, which is a protein highly expressed in liver macrophages.

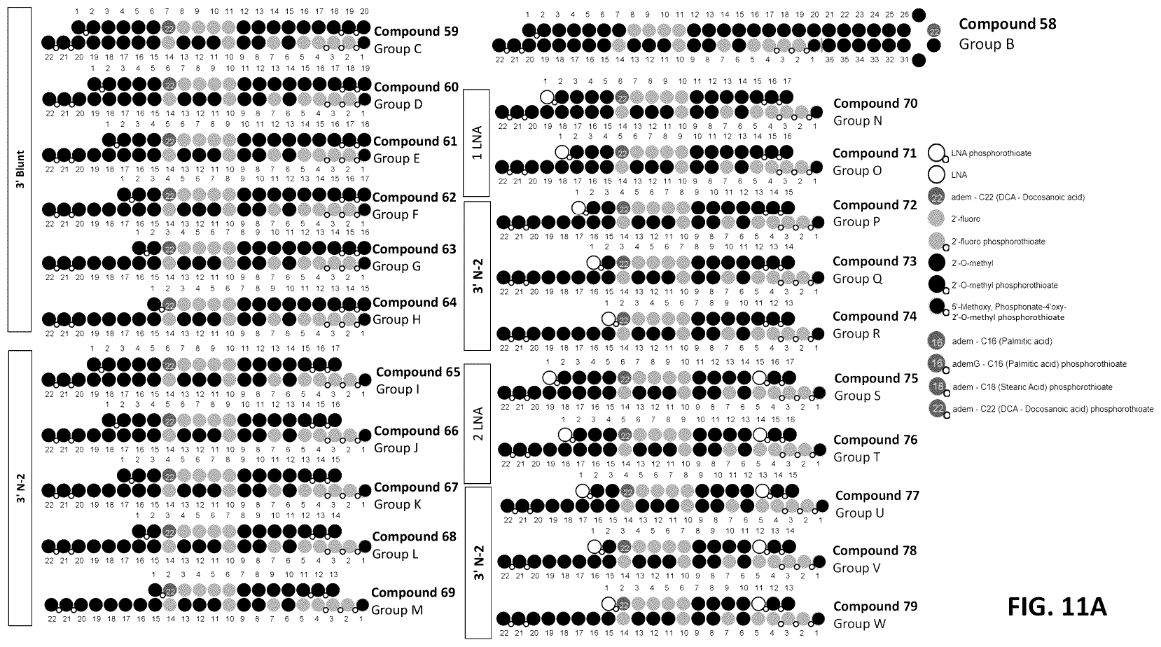

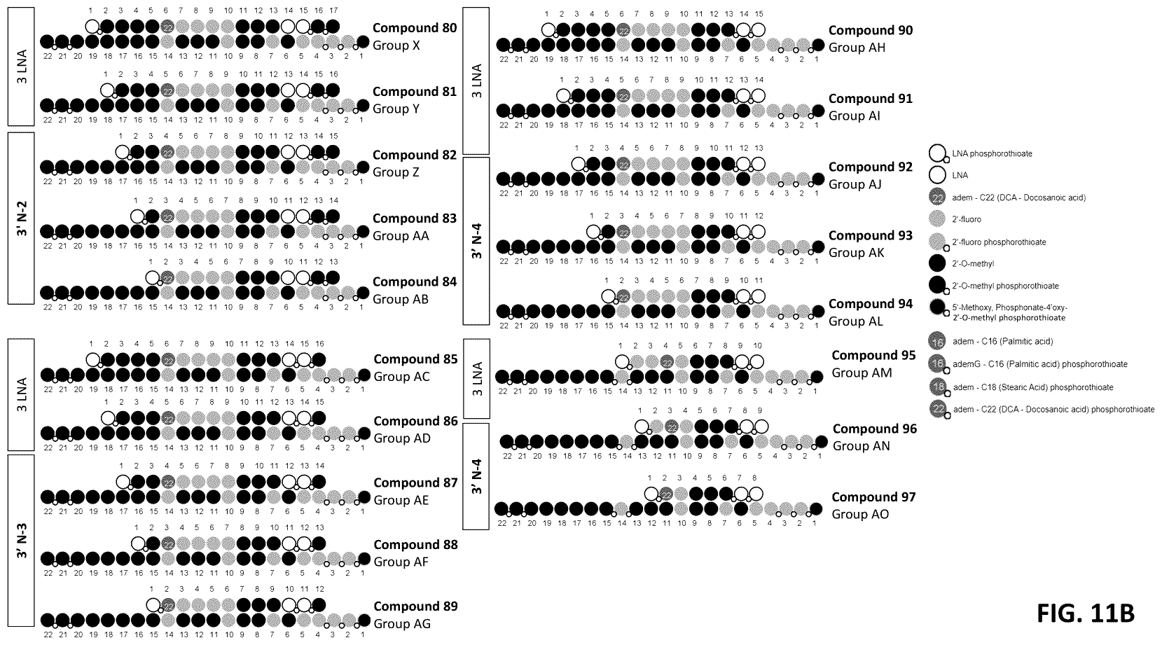

Comparison was made to an oligonucleotide-lipid conjugate evaluated in previous studies having the structure of Compound 58 as shown in FIG. 11A. Compound 58 contains a stem-loop at the 3′ terminus of the sense strand and a 2-nucleotide overhang at the 3′ terminus of the antisense strand. The stem-loop contains a tetraloop having the nucleotide sequence 5′-GAAA-3′, a stem of 6 nt in length, and a C22 lipid conjugated to the second nucleotide of the tetraloop (i.e., the underlined “A” of the 5′-GAAA-3′ tetraloop).

Oligonucleotide-lipid conjugates that were evaluated included those having the structures of Compounds 59-97 as shown in FIGS. 11A-11B and further described below:
(i) Compounds 59-64 as shown in FIG. 11A contain a sense strand having a blunt end at the 3′ terminus and a truncation at the 5′ terminus of 0-5 nucleotides respectively, yielding an antisense strand having an overhang at the 3′ terminus of 2-nt to 7-nt respectively. Each contains a C22 lipid conjugate positioned 14-nt from the 3′ terminus of the sense strand.
(ii) Compounds 65-69 as shown in FIG. 11A contain a sense strand having a 2-nt truncation at the 3′ terminus and a truncation at the 5′ terminus of 1-5 nucleotides respectively, yielding an antisense strand having an overhang at the 5′ terminus of 2-nt and at the 3′ terminus of 3-nt to 7-nt respectively. Each contains a C22 lipid conjugate positioned 12-nt from the 3′ terminus of the sense strand.
(iii) Compounds 70-74 as shown in FIG. 11A contain a sense strand having a 2-nt truncation at the 3′ terminus and a truncation at the 5′ terminus of 1-5 nucleotides respectively, yielding an antisense strand having an overhang at the 5′ terminus of 2-nt and at the 3′ terminus of 3-nt to 7-nt respectively. Each contains a sense strand having a C22 lipid conjugate positioned 12-nt from the 3′ terminus and a single LNA at the 5′ terminus.
(iv) Compounds 75-79 as shown in FIG. 11A contain a sense strand having a 2-nt truncation at the 3′ terminus and a truncation at the 5′ terminus of 1-5 nucleotides respectively, yielding an antisense strand having an overhang at the 5′ terminus of 2-nt and at the 3′ terminus of 3-nt to 7-nt respectively. Each contains a sense strand having a C22 lipid conjugate positioned 12-nt from the 3′ terminus, and two LNAs (one positioned at the 5′ terminus and one positioned 3-nt from the 3′ terminus).
(v) Compounds 80-84 as shown in FIG. 11B contain a sense strand having a 2-nt truncation at the 3′ terminus and a truncation at the 5′ terminus of 1-5 nucleotides respectively, yielding an antisense strand having an overhang at the 5′ terminus of 2-nt and at the 3′ terminus of 3-nt to 7-nt respectively. Each contains a sense strand having a C22 lipid conjugate positioned 12-nt from the 3′ terminus, and three LNAs (one positioned at the 5′ terminus, one positioned 3-nt from the 3′ terminus, and one positioned 4-nt from the 3′ terminus).
(vi) Compounds 85-89 as shown in FIG. 11B contain a sense strand having a 3-nt truncation at the 3′ terminus and a truncation at the 5′ terminus of 1-5 nucleotides respectively, yielding an antisense strand having an overhang at the 5′ terminus of 3-nt and at the 3′ terminus of 3-nt to 7-nt respectively. Each contains a sense strand having a C22 lipid conjugate positioned 11-nt from the 3′ terminus, and three LNAs (one positioned at the 5′ terminus, one positioned 2-nt from the 3′ terminus, and one positioned 3-nt from the 3′ terminus). An additional PS backbone linkage was installed such that two PS linkages were present at the 3′ terminus of the sense strand.
(vi) Compounds 90-94 as shown in FIG. 11B contain a sense strand having a 4-nt truncation at the 3′ terminus and a truncation at the 5′ terminus of 1-5 nucleotides respectively, yielding an antisense strand having an overhang at the 5′ terminus of 4-nt and at the 3′ terminus of 3-nt to 7-nt respectively. Each contains a sense strand having a C22 lipid conjugate positioned 10-nt from the 3′ terminus, and three LNAs (one positioned at the 5′ terminus, one positioned at the 3′ terminus, and one positioned 2-nt from the 3′ terminus). An additional PS backbone linkage was installed such that two PS linkages were present at the 3′ terminus of the sense strand.
(vii) Compounds 95-97 as shown in FIG. 11B contain a sense strand having a 4-nt truncation at the 3′ terminus and a truncation at the 5′ terminus of 6-nt to 8-nt respectively, yielding an antisense strand having an overhang at the 5′ terminus of 4-nt and at the 3′ terminus of 8-nt to 10-nt respectively. Each contains a sense strand having a C22 lipid conjugate positioned 7-nt from the 3′ terminus, and three LNAs (one positioned at the 5′ terminus, one positioned at the 3′ terminus, and one positioned 2-nt from the 3′ terminus). An additional PS backbone linkage was installed such that two PS linkages were present at the 3′ terminus of the sense strand.

To evaluate the RNAi oligonucleotide-lipid conjugates, CD-1 female mice (having a weight of 18-20 g) were given a single subcutaneous (s.c.) injection of 15 mg/kg oligonucleotide-lipid conjugate formulated in PBS. The RNAi oligonucleotide-lipid conjugates administered for each of the treatment groups are outlined in Table 9. Group A were control mice that received PBS only.

TABLE 9
Lipid-conjugated RNAi oligonucleotides
administered to animal groups A-AO

| | | Sense | | Antisense | |
|---|---|---|---|---|---|
| Group | Compound | SEQ ID NOS | | SEQ ID NOs | |
| # | ID | Unmod | Mod | Unmod | Mod |
| A | — | — | — | — | — |
| B | 58 | 13 | 103 | 58 | 148 |
| C | 59 | 14 | 104 | 59 | 149 |
| D | 60 | 15 | 105 | 60 | 150 |
| E | 61 | 16 | 106 | 61 | 151 |
| F | 62 | 17 | 107 | 62 | 152 |
| G | 63 | 18 | 108 | 63 | 153 |
| H | 64 | 19 | 109 | 64 | 154 |
| I | 65 | 20 | 110 | 65 | 155 |
| J | 66 | 21 | 111 | 66 | 156 |
| K | 67 | 22 | 112 | 67 | 157 |
| L | 68 | 23 | 113 | 68 | 158 |
| M | 69 | 24 | 114 | 69 | 159 |
| N | 70 | 25 | 115 | 70 | 160 |
| O | 71 | 26 | 116 | 71 | 161 |
| P | 72 | 27 | 117 | 72 | 162 |
| Q | 73 | 28 | 118 | 73 | 163 |
| R | 74 | 29 | 119 | 74 | 164 |
| S | 75 | 30 | 120 | 75 | 165 |
| T | 76 | 31 | 121 | 76 | 166 |
| U | 77 | 32 | 122 | 77 | 167 |
| V | 78 | 33 | 123 | 78 | 168 |
| W | 79 | 34 | 124 | 79 | 169 |
| X | 80 | 35 | 125 | 80 | 170 |
| Y | 81 | 36 | 126 | 81 | 171 |
| Z | 82 | 37 | 127 | 82 | 172 |
| AA | 83 | 38 | 128 | 83 | 173 |
| AB | 84 | 39 | 129 | 84 | 174 |
| AC | 85 | 40 | 130 | 85 | 175 |
| AD | 86 | 41 | 131 | 86 | 176 |
| AE | 87 | 42 | 132 | 87 | 177 |
| AF | 88 | 43 | 133 | 88 | 178 |
| AG | 89 | 44 | 134 | 89 | 179 |
| AH | 90 | 45 | 135 | 90 | 180 |
| AI | 91 | 46 | 136 | 91 | 181 |
| AJ | 92 | 47 | 137 | 92 | 182 |
| AK | 93 | 48 | 138 | 93 | 183 |
| AL | 94 | 49 | 139 | 94 | 184 |
| AM | 95 | 50 | 140 | 95 | 185 |
| AN | 96 | 51 | 141 | 96 | 186 |
| AO | 97 | 52 | 142 | 97 | 187 |

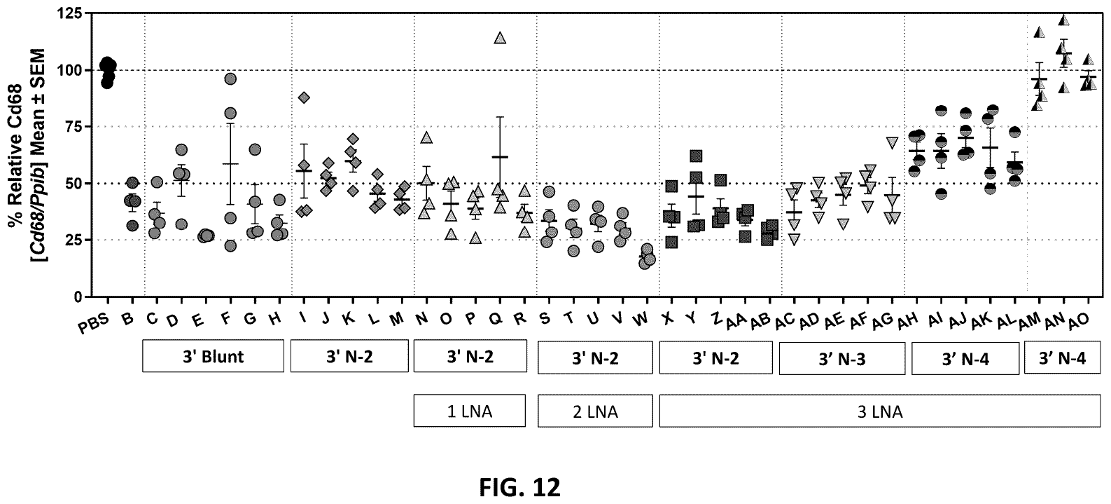

Target knockdown was assessed 7 days post-injection. RNA was extracted from liver tissue and murine CD68 mRNA levels were determined by qPCR (normalized to endogenous housekeeping gene Ppib). The qPCR was performed using PrimeTime™ qPCR Probe Assays. The percentage of murine CD68 mRNA remaining in the samples from treated mice was determined using the 2-ΔΔCt (“delta-delta Ct”) method (Livak and Schmittgen (2001) METHODS 25:402-408). Percent CD68 mRNA expression in the liver as measured for each treatment group is shown in FIG. 12. Silencing of CD68 mRNA expression was observed for most treatment groups. Overall, truncations at the 5′ terminus of the sense strand were well-tolerated. All molecules in this Example comprising a 2-nt or 3-nt overhang at the 5′ end of the antisense strand reduced CD68 mRNA expression. Compounds 90-94 comprising a 4-nt overhang at the 5′ end of the antisense strand reduced expression of CD68 mRNA expression.

### Example 10: RNAi Oligonucleotide-lipid Conjugates Targeting the Liver

RNAi oligonucleotide-lipid conjugates were evaluated to determine the effect of inclusion of LNAs and positioning of a lipid conjugate on specific targeting of activity in liver tissue. Each oligonucleotide-lipid conjugate tested comprised an antisense strand having a region of complementarity to mRNA encoding Aldh2, which is a protein expressed in liver and other tissues.

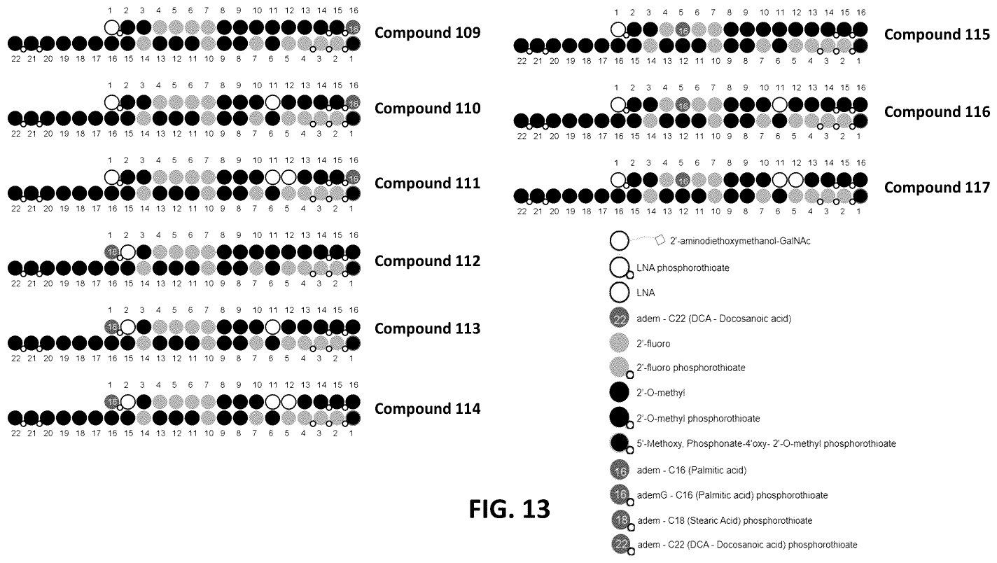

Oligonucleotide-lipid conjugates that were evaluated included those having the structures of Compounds 109-117 as shown in FIG. 13. Each of the compounds contain a sense strand having a blunt end at the 3′ terminus, a truncation at the 5′ terminus resulting in an antisense strand having a 6-nt overhang at the 3′ terminus, a C16 lipid conjugate, and at least one LNA. The positioning of the C16 lipid conjugate and the number of LNAs varied per compound as further described below:
(i) Compounds 109-111 contain a C16 lipid conjugate positioned at the 3′ terminus of the sense strand and an LNA positioned at the 5′ terminus of the sense strand. Compound 110 contains an LNA at position 11 of the sense strand. Compound 111 contains an LNA at positions 11 and 12 of the sense strand.
(ii) Compounds 112-114 contain a C16 lipid conjugate positioned at the 5′ terminus of the sense strand and an LNA at position 2 of the sense strand. Compound 113 contains an LNA at position 11 of the sense strand. Compound 114 contains an LNA at positions 11 and 12 of the sense strand.
(iii) Compounds 115-117 contain a C16 lipid conjugate at position 5 of the sense strand and an LNA at the 5′ terminus of the sense strand. Compound 116 contains an LNA at position 11 of the sense strand. Compound 117 contains an LNA at positions 11 and 12 of the sense strand.

To evaluate the RNAi oligonucleotide-lipid conjugates, mice were given a single subcutaneous (s.c.) injection of 10 mg/kg oligonucleotide-lipid conjugate formulated in PBS. The RNAi oligonucleotide-lipid conjugate administered to each animal group is outlined in Table 10. Group A were control mice that received PBS only.

TABLE 10
Lipid-conjugated RNAi oligonucleotides
administered to animal groups A-J

| | | Sense | | Antisense | |
|---|---|---|---|---|---|
| Group | Compound | SEQ ID NOS | | SEQ ID NOS | |
| # | ID | Unmod | Mod | Unmod | Mod |
| A | — | — | — | — | — |
| B | 109 | 241 | 260 | 250 | 269 |
| C | 110 | 242 | 261 | 251 | 270 |
| D | 111 | 243 | 262 | 252 | 271 |
| E | 112 | 244 | 263 | 253 | 272 |
| F | 113 | 245 | 264 | 254 | 273 |
| G | 114 | 246 | 265 | 255 | 274 |
| H | 115 | 247 | 266 | 256 | 275 |
| I | 116 | 248 | 267 | 257 | 276 |
| J | 117 | 249 | 268 | 258 | 277 |

Target knockdown was assessed 7 days post-injection. RNA was extracted from various tissues and murine ALDH2 mRNA levels were determined by qPCR (normalized to endogenous housekeeping gene Ppib). The qPCR was performed using PrimeTime™ qPCR Probe Assays, which consisted of a primer pair and fluorescently labeled 5′ nuclease probe specific to murine ALDH2 mRNA. The percentage of murine ALDH2 mRNA remaining in the samples from treated mice was determined using the 2-ΔΔCt (“delta-delta Ct”) method (Livak and Schmittgen (2001) METHODS 25:402-408).

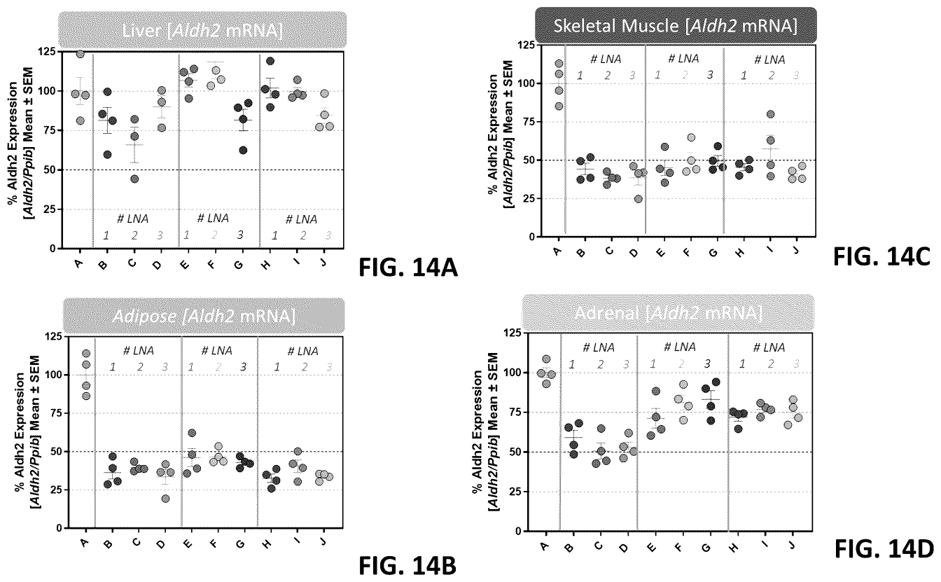

Percent ALDH2 mRNA expression in the liver, skeletal muscle, adipose, and adrenal as measured for each treatment group is respectively shown in FIGS. 14A-14D. Silencing of ALDH2 mRNA expression was observed in all tissues evaluated for most treatment groups.

### Example 11: RNAi Oligonucleotide-Lipid Conjugates Having a Double Overhang Produce Silencing of mRNA Encoding a Liver Target

It was evaluated if an RNAi oligonucleotide-lipid conjugate having an overhang at both the 5′ terminus and 3′ terminus of the antisense strand would provide similar silencing activity against a liver target as a conjugate having a blunt end. Silencing of mRNA expression of four liver targets was evaluated, including STAT3, SLC25A1, HMGB1, and ALDH2.

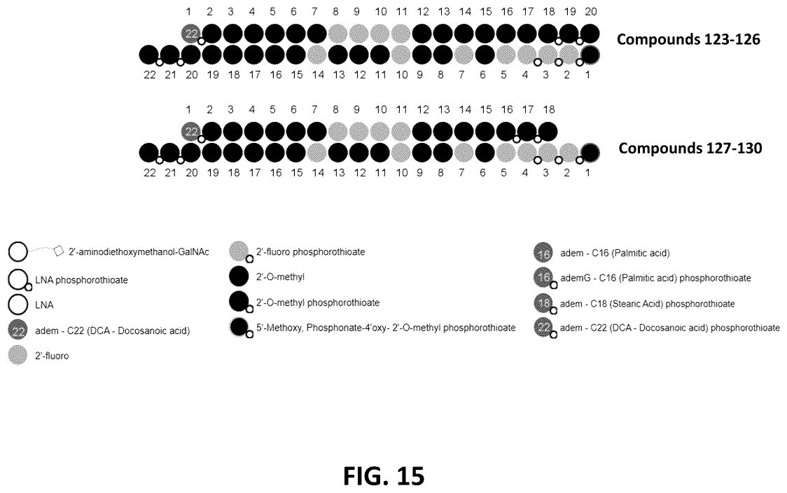

The RNAi oligonucleotide-lipid conjugates evaluated had the structures of Compounds 123-126 as shown in FIG. 15. Compounds 123-126 have an antisense strand respectively having a region of complementarity to mRNA encoding STAT3, SLC25A1, HMGB1, and ALDH2, a 2-nt overhang at the 3′ terminus, and a blunt end at the 5′ terminus. Compounds 127-130 have an antisense strand respectively having a region of complementarity to mRNA encoding STAT3, SLC25A1, HMGB1, and ALDH2 and a 2-nt overhang at both the 5′ terminus and 3′ terminus.

To evaluate the RNAi oligonucleotide-lipid conjugates, mice were given a single subcutaneous (s.c.) injection of 15 mg/kg oligonucleotide-lipid conjugate formulated in PBS. The RNAi oligonucleotide-lipid conjugates administered for each of the animal groups are outlined in Table 11. Group A were control mice that received PBS only.

TABLE 11
Lipid-conjugated RNAi oligonucleotides directed
to mRNA targets expressed in the liver

| | | | Sense SEQ ID | | Antisense SEQ | |
|---|---|---|---|---|---|---|
| Group | Compound | mRNA | NOs | | ID NOs | |
| # | ID | target | Unmod | Mod | Unmod | Mod |
| A | — | — | — | — | — | — |
| B1 | 123 | STAT3 | 278 | 294 | 286 | 302 |
| B2 | 124 | SLC25A1 | 279 | 295 | 287 | 303 |
| B3 | 125 | HMGB1 | 280 | 296 | 288 | 304 |
| B4 | 126 | ALDH2 | 281 | 297 | 289 | 305 |
| C1 | 127 | STAT3 | 282 | 298 | 290 | 306 |
| C2 | 128 | SLC25A1 | 283 | 299 | 291 | 307 |
| C3 | 129 | HMGB1 | 284 | 300 | 292 | 308 |
| C4 | 130 | ALDH2 | 285 | 301 | 293 | 309 |

Target knockdown was assessed 14 days post-injection. RNA was extracted from liver tissue and murine STAT3, SLC25A1, HMGB1, and ALDH2 mRNA levels were determined by qPCR (normalized to endogenous housekeeping gene Ppib). The qPCR was performed using PrimeTime™ qPCR Probe Assays. The percentage of target mRNA remaining in the samples from treated mice was determined using the 2-ΔΔCt (“delta-delta Ct”) method (Livak and Schmittgen (2001) METHODS 25:402-408).

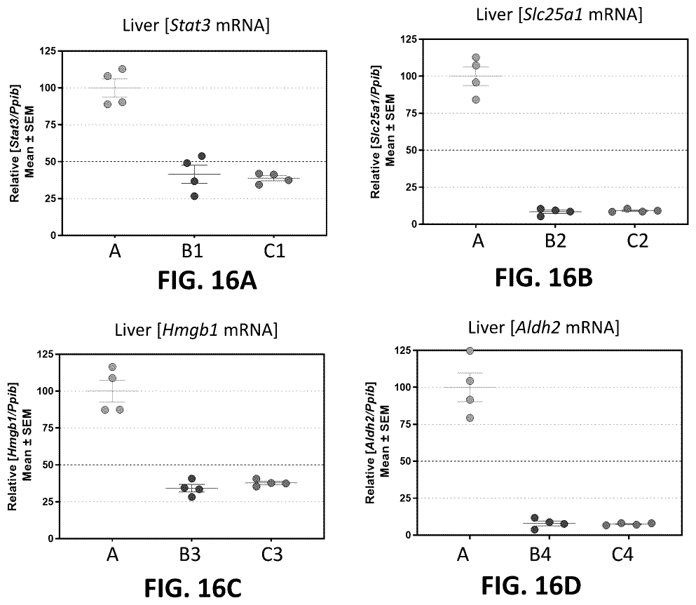

As shown in FIGS. 16A-16B, similar levels of reduction of mRNA encoding STAT3 (FIG. 16A), SLC25A1 (FIG. 16B), HMGB1 (FIG. 16C), and ALDH2 (FIG. 16D) was observed for oligonucleotide-lipid conjugate having a blunt-end at the 5′ terminus of the antisense strand or having a double overhang at the 5′ and 3′ termini of the antisense strand.

### Example 12: Tetraloop Passenger Truncations with and without Phosphorothioate Linkage on Exposed Guide for Inhibition of a Neuronal Target Gene in the CNS

RNAi oligonucleotide-lipid conjugates were evaluated to determine the effect of passenger strand truncations with and without phosphorothioate linkages on the exposed guide strand in neurons in oligonucleotides having a tetraloop. Each oligonucleotide-lipid conjugate tested comprised an antisense strand having a region of complementarity to mRNA encoding TUBB3.

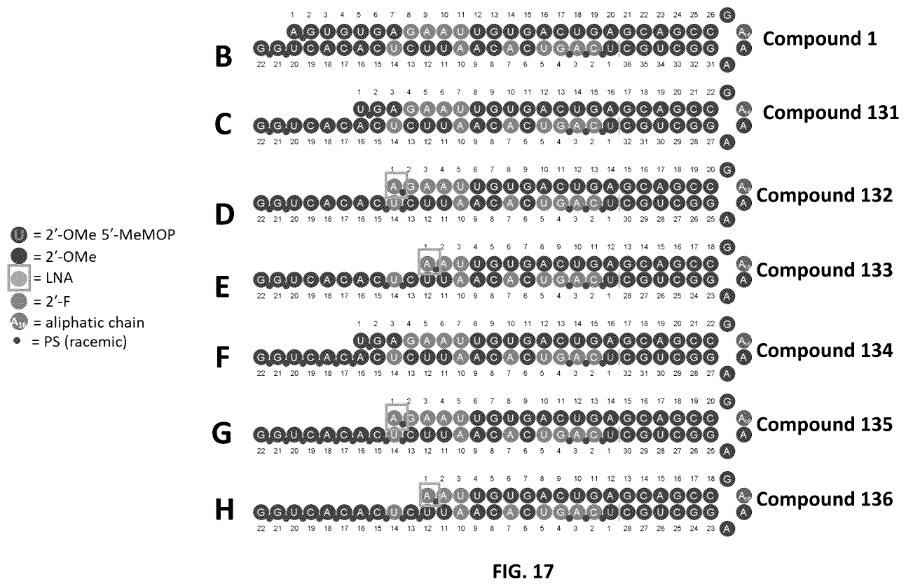

Oligonucleotide-lipid conjugates that were evaluated included those having the structures of Compounds 131-136 as shown in FIG. 17, compared to the parent oligonucleotide-lipid conjugate evaluated in previous studies (Compound 1, described above). Each of Compounds 131-136 contain a sense strand having a tetraloop at the 3′ terminus and a C16 lipid conjugated on the second nucleotide of the tetraloop. The compounds comprise different passenger strand lengths and phosphorothioate linkages on each compound as further described below:
(i) Compound 131 comprises a 32-nucleotide sense strand with a 4-nt truncation (i.e. a 6-nt antisense strand overhang at the 3′ terminus) and comprises phosphorothioate linkages between positions 1 and 2 of the sense strand and between positions 1 and 2, 2 and 3, 3 and 4, 20 and 21, and 21 and 22 of the antisense strand.
(ii) Compound 132 comprises a 30-nucleotide sense strand with a 6-nt truncation (i.e. an 8-nt antisense strand overhang at the 3′ terminus) and comprises phosphorothioate linkages between positions 1 and 2 of the sense strand and between positions 1 and 2, 2 and 3, 3 and 4, 13 and 14, 14 and 15, 20 and 21, and 21 and 22 of the antisense strand. Additionally, the sense strand comprises an LNA at position 1.
(iii) Compound 133 comprises a 28-nucleotide sense strand with an 8-nt truncation (i.e. a 10-nt antisense strand overhang at the 3′ terminus) and comprises phosphorothioate linkages between positions 1 and 2 of the sense strand and between positions 1 and 2, 2 and 3, 3 and 4, 13 and 14, 14 and 15, 20 and 21, and 21 and 22 of the antisense strand. Additionally, the sense strand comprises an LNA at position 1.
(iii) Compound 134 comprises a 32-nucleotide sense strand with a 4-nt truncation (i.e. a 6-nt antisense strand overhang at the 3′ terminus) and comprises phosphorothioate linkages between positions 1 and 2 of the sense strand and between positions 1 and 2, 2 and 3, 3 and 4, 16 and 17, 17 and 18, 18 and 19, 19 and 20, 20 and 21, and 21 and 22 of the antisense strand.
(iv) Compound 135 comprises a 30-nucleotide sense strand with a 6-nt truncation (i.e. an 8-nt antisense strand overhang at the 3′ terminus) and comprises phosphorothioate linkages between positions 1 and 2 of the sense strand and between positions 1 and 2, 2 and 3, 3 and 4, 13 and 14, 14 and 15, 15 and 16, 16 and 17, 17 and 18, 18 and 19, 19 and 20, 20 and 21, and 21 and 22 of the antisense strand. Additionally, the sense strand comprises an LNA at position 1.
(v) Compound 136 comprises a 28-nucleotide sense strand with an 8-nt truncation (i.e. a 10-nt antisense strand overhang at the 3′ terminus) and comprises phosphorothioate linkages between positions 1 and 2 of the sense strand and between positions 1 and 2, 2 and 3, 3 and 4, 12 and 13, 13 and 14, 14 and 15, 15 and 16, 16 and 17, 17 and 18, 18 and 19, 19 and 20, 20 and 21, and 21 and 22 of the antisense strand. Additionally, the sense strand comprises an LNA at position 1.

To evaluate the RNAi oligonucleotide-lipid conjugates, 6 to 10 week old C57BL6 female mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 12. Group A were control mice that received artificial cerebrospinal fluid (aCSF) only.

TABLE 12
Lipid-conjugated RNAi oligonucleotides
administered to animal groups A-H

| | | Sense | | Antisense | |
|---|---|---|---|---|---|
| Group | Compound | SEQ ID NOS | | SEQ ID NOs | |
| # | ID | Unmod | Mod | Unmod | Mod |
| A | aCSF | — | — | — | — |
| B | 1 | 310 | 338 | 324 | 352 |
| C | 131 | 527 | 530 | 324 | 352 |
| D | 132 | 528 | 531 | 324 | 533 |
| E | 133 | 529 | 532 | 324 | 533 |
| F | 134 | 527 | 530 | 324 | 534 |
| G | 135 | 528 | 531 | 324 | 535 |
| H | 136 | 529 | 532 | 324 | 536 |

Target knockdown was assessed 28 days post-injection. RNA was extracted from various tissues and murine TUBB3 mRNA levels were determined as described in Example 2.

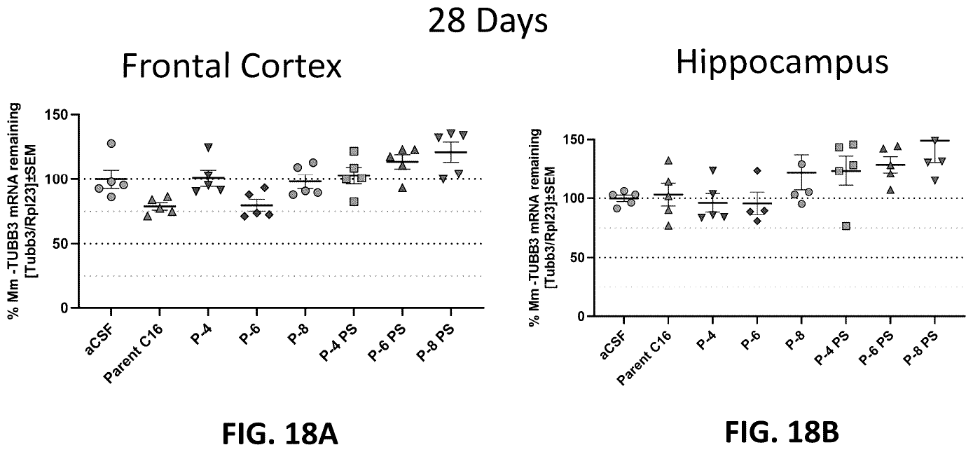

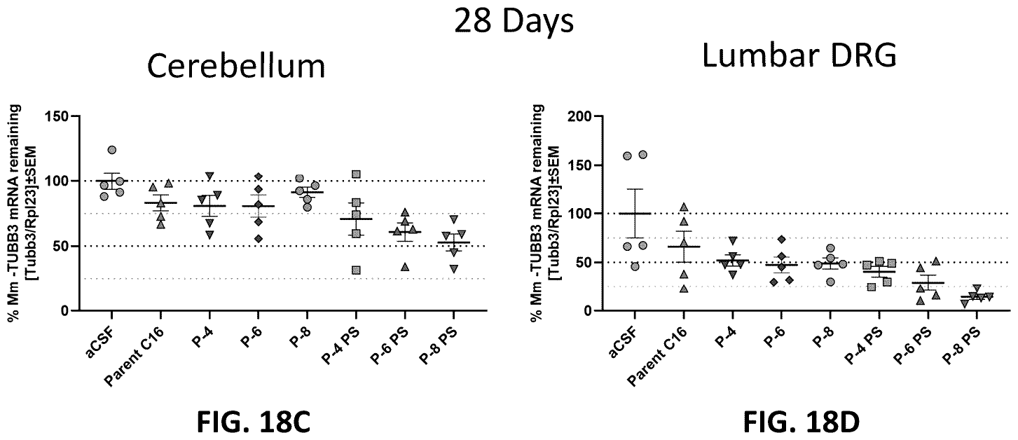

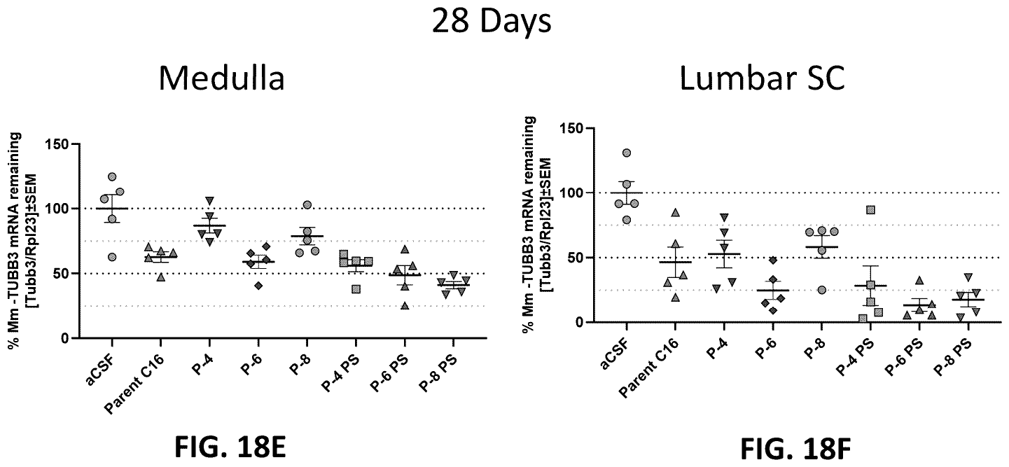

Percent TUBB3 mRNA expression in the frontal cortex, hippocampus, cerebellum, lumbar dorsal root ganglion, medulla, and lumbar spinal cord as measured for each treatment group is respectively shown in FIGS. 18A-18F. Silencing of TUBB3 mRNA expression was observed in cerebellum, lumbar DRG, medulla, and lumbar spinal cord. Particularly, Compound 136 (comprising a P-8 truncation, i.e., a 10-nt antisense overhang, with phosphorothioate linkages on the exposed antisense strand) demonstrated increased inhibition in the lumbar DRG with restricted inhibition beyond the medulla demonstrating a platform for inhibiting target gene knockdown in the lumbar DRG.

### Example 13: 5′ Truncations with Locked Nucleic Acids on the Sense Strand for Inhibition of a Neuronal Target Gene in the CNS

RNAi oligonucleotide-lipid conjugates were evaluated to determine the effect of blunt-end passenger truncations with locked nucleic acids on the guide strand in neurons. Each oligonucleotide-lipid conjugate tested comprised an antisense strand having a region of complementarity to mRNA encoding TUBB3.

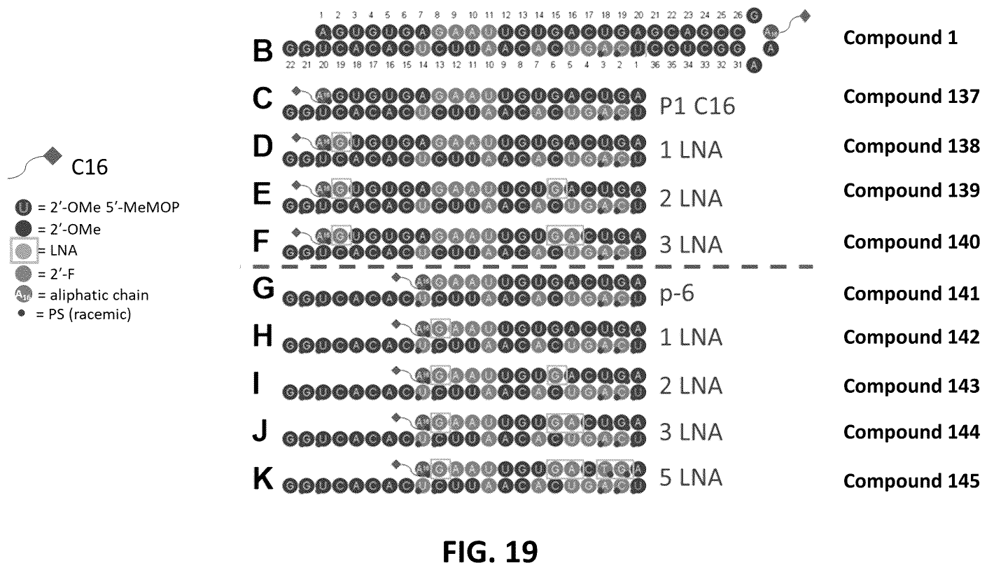

Oligonucleotide-lipid conjugates that were evaluated included those having the structures of Compounds 138-145 as shown in FIG. 19, compared to the parent oligonucleotide-lipid conjugate evaluated in previous studies (Compound 1, described above) and Compound 137. Compound 137 contains a blunt-end at the 3′ terminus of the sense strand, a 2-nt overhang at the 3′ terminus of the antisense strand, and a C16 lipid conjugated to the 5′ terminal nucleotide of the sense strand. Each of Compounds 138-145 contain a blunt-end at the 3′ terminus of the sense strand, a C16 lipid conjugated at the 5′ terminal of the sense strand (position 1), and phosphorothioate linkages between positions: 1 and 2, 18 and 19, 19 and 20, of the sense strand; 1 and 2, 2 and 3, 3 and 4, 20 and 21, 21 and 22, of the antisense strand. Compounds 141-145 comprise a truncation at the 5′ terminus of the sense strand resulting in 6-nt truncation (i.e. an 8-nt antisense strand overhang at the 3′ terminus). Additionally, Compounds 141-145 comprise two phosphorothioate linkages flanking the nucleotide at position 14 (from 5′ to 3′) of the antisense strand (i.e., phosphorothioate linkages between positions 13 and 14, and 14 and 15, of the antisense strand). The compounds comprise different positions of locked nucleic acids as further described below:
(i) Compound 141 contains no locked nucleic acids (LNAs).
(ii) Compounds 138 and 142 contain an LNA at position 2 of the sense strand.
(iii) Compound 139 contains an LNA at positions 2 and 15.
(iv) Compound 140 contains an LNA at positions 2, 15, and 16.
(v) Compound 143 contains an LNA at positions 2 and 9.
(vi) Compounds 144 contains an LNA at positions 2, 9, and 10.
(vii) Compound 145 contains an LNA at positions 2, 9, 10, 12, and 13.

To evaluate the RNAi oligonucleotide-lipid conjugates, mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 13. Group A were control mice that received artificial cerebrospinal fluid (aCSF) only.

TABLE 13
Lipid-conjugated RNAi oligonucleotides
administered to animal groups A-K

| | | Sense | | Antisense | |
|---|---|---|---|---|---|
| Group | Compound | SEQ ID NOS | | SEQ ID NOS | |
| # | ID | Unmod | Mod | Unmod | Mod |
| A | aCSF | | | | |
| B | 1 | 310 | 338 | 324 | 352 |
| C | 137 | 372 | 386 | 324 | 352 |
| D | 138 | 372 | 539 | 324 | 352 |
| E | 139 | 372 | 540 | 324 | 352 |
| F | 140 | 372 | 541 | 324 | 352 |
| G | 141 | 537 | 542 | 324 | 533 |
| H | 142 | 537 | 543 | 324 | 533 |
| I | 143 | 537 | 544 | 324 | 533 |
| J | 144 | 537 | 545 | 324 | 533 |
| K | 145 | 538 | 546 | 324 | 533 |

Target knockdown was assessed 28 days post-injection. RNA was extracted from various tissues and murine TUBB3 mRNA levels were determined as described in Example 2.

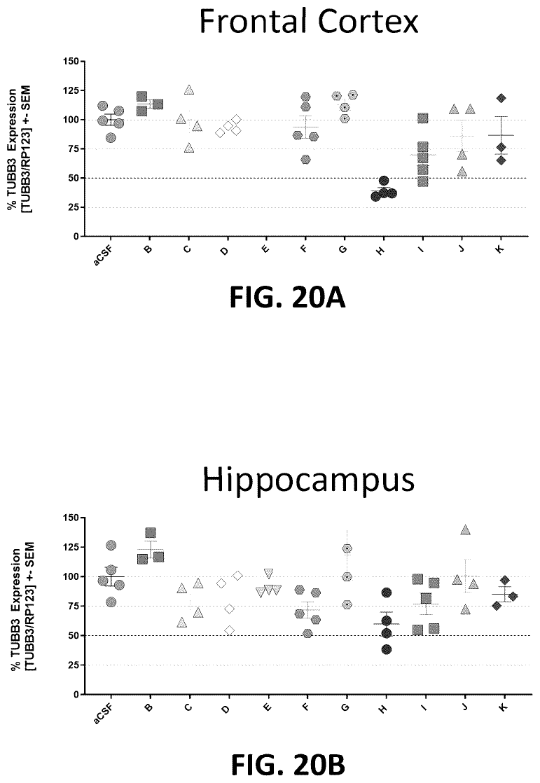

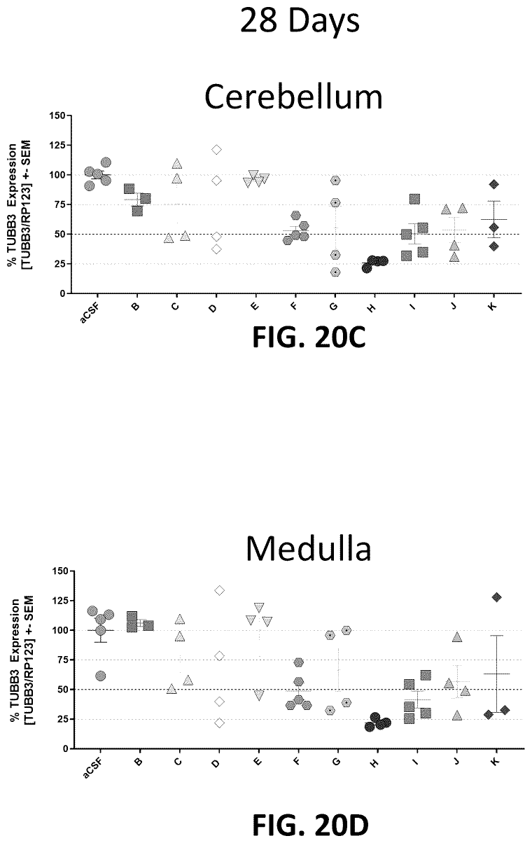

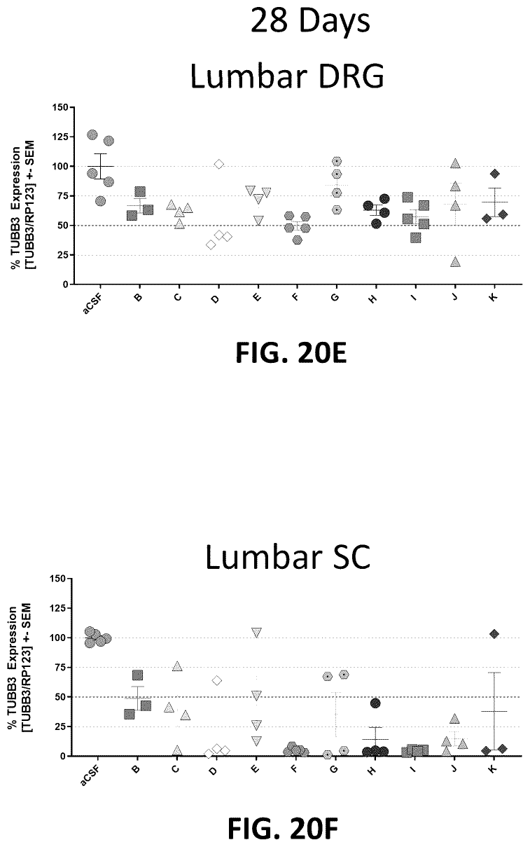

Percent TUBB3 mRNA expression in the frontal cortex, hippocampus, cerebellum, medulla, lumbar dorsal root ganglion, and lumbar spinal cord as measured for each treatment group is respectively shown in FIGS. 20A-20F. Silencing of TUBB3 mRNA expression was observed in all tissues evaluated. Inclusion of at least one LNA with a P-6 sense strand truncation enhanced knockdown efficiency in most of the tissues evaluated (e.g., Compound 142) and inclusion of 2 LNAs enhanced knockdown further.

### Example 14: 5′ C16 Lipid Conjugated Blunt-End Oligonucleotides with Sense Strand Locked Nucleic Acids for Inhibition of a Neuronal Target Gene in the CNS

RNAi oligonucleotide-lipid conjugates were evaluated to determine the effect of blunt-end oligonucleotides with locked nucleic acids on the guide strand in neurons. Each oligonucleotide-lipid conjugate tested comprised an antisense strand having a region of complementarity to mRNA encoding TUBB3.

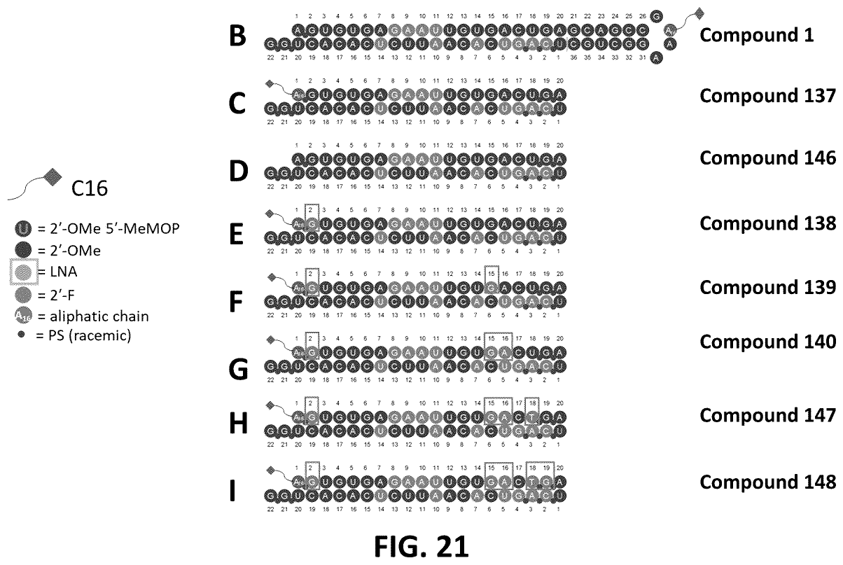

Oligonucleotide-lipid conjugates that were evaluated included those having the structures of Compounds 138-140, and 146-148 as shown in FIG. 21, compared to the parent oligonucleotide-lipid conjugate evaluated in previous studies (Compounds 1 and 137, described above). Each of Compounds 138-140 and 147-148 contain a blunt-end at the 3′ terminus of the sense strand and a C16 lipid conjugated at the 5′ terminal nucleotide of the sense strand. Compound 146 contains a blunt-end at the 3′ terminus of the sense strand and no lipid conjugated at the 5′ terminus of the sense strand. Each of Compounds 138-140 and 147-148 comprise phosphorothioate linkages between positions: 1 and 2, 18 and 19, and 19 and 20, of the sense strand; 1 and 2, 2 and 3, 3 and 4, 20 and 21, and 21 and 22, of the antisense strand. The compounds comprise different positions of locked nucleic acids as further described below:
(i) Compounds 137, and 146 contain no locked nucleic acids.
(ii) Compound 138 contains an LNA at position 2 of the sense strand.
(iii) Compound 139 contains an LNA at positions 2 and 15.
(iv) Compound 140 contains an LNA at positions 2, 15, and 16.
(v) Compound 145 contains an LNA at positions 2, 15, 16, and 18.
(vi) Compound 148 contains an LNA at positions 2, 15, 16, 18, and 19.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 14. Group A were control mice that received artificial cerebrospinal fluid (aCSF) only.

TABLE 14
Lipid-conjugated RNAi oligonucleotides
administered to animal groups A-I

| Group | Compound | Sense SEQ ID NOS | | Antisense SEQ ID NOs | |
|---|---|---|---|---|---|
| # | ID | Unmod | Mod | Unmod | Mod |
| A | aCSF | | | | |
| B | 1 | 310 | 338 | 324 | 352 |
| C | 137 | 372 | 386 | 324 | 352 |
| D | 146 | 372 | 547 | 324 | 352 |
| E | 138 | 372 | 539 | 324 | 352 |
| F | 139 | 372 | 540 | 324 | 352 |
| G | 140 | 372 | 541 | 324 | 352 |
| H | 147 | 439 | 548 | 324 | 352 |
| I | 148 | 439 | 461 | 324 | 352 |

Target knockdown was assessed 28 days post-injection. RNA was extracted from various tissues and murine TUBB3 mRNA levels were determined as described in Example 2.

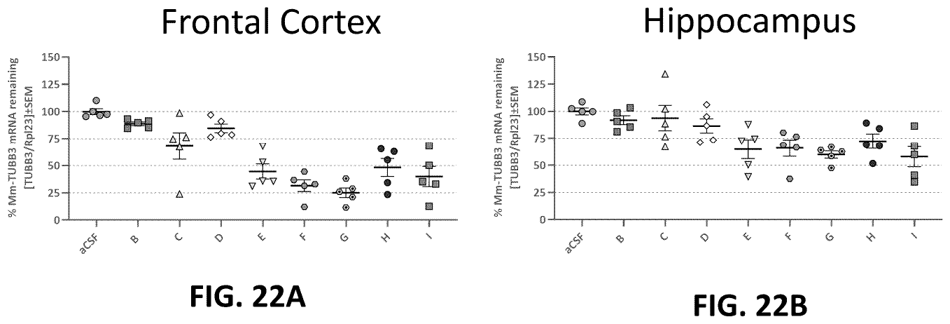

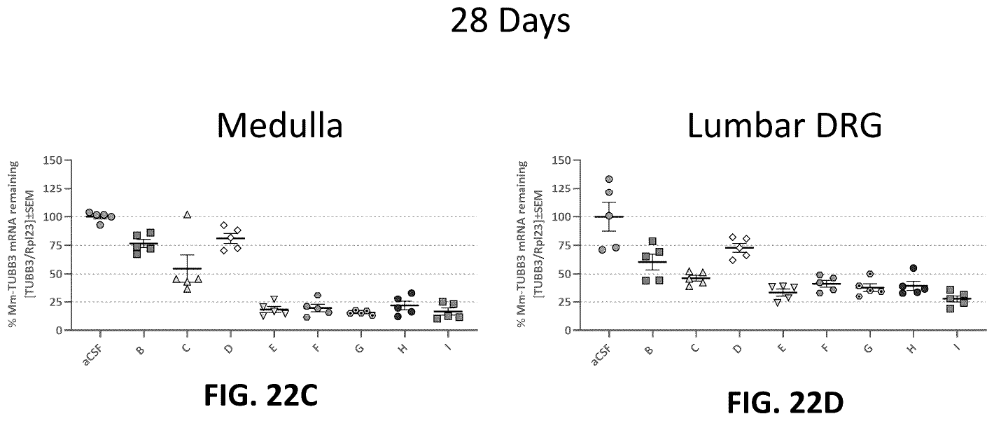

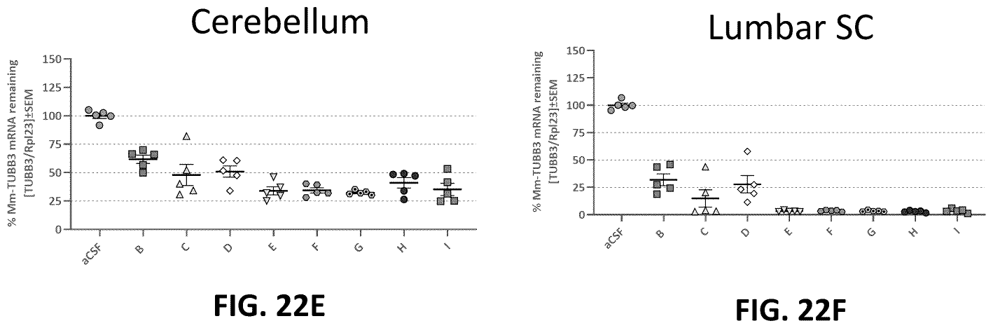

Percent TUBB3 mRNA expression in the frontal cortex, hippocampus, medulla, lumbar dorsal root ganglion, cerebellum, and lumbar spinal cord as measured for each treatment group is respectively shown in FIGS. 22A-22F. Silencing of TUBB3 mRNA expression was observed in all tissues evaluated for most treatment groups. Conjugation of a lipid (i.e., C16) on the 5′ end of the sense strand enhanced neuronal knockdown (see e.g., Compound 146 compared to Compounds E-I). Additionally, inclusion of at least 1 LNA increased knockdown activity of oligonucleotides in deeper (e.g., frontal cortex and hippocampus) tissue compared to compounds without an LNA, with inclusion of 2 to 4 LNAs further increasing knockdown activity.

### Example 15: 5′C16 Lipid Conjugated P-4 Truncated Sense Strand with Locked Nucleic Acids for Inhibition a Neuronal Target Gene in the CNS

RNAi oligonucleotide-lipid conjugates were evaluated to determine the effect of blunt-end passenger truncations with locked nucleic acids on the guide strand in neurons. Each oligonucleotide-lipid conjugate tested comprised an antisense strand having a region of complementarity to mRNA encoding TUBB3.

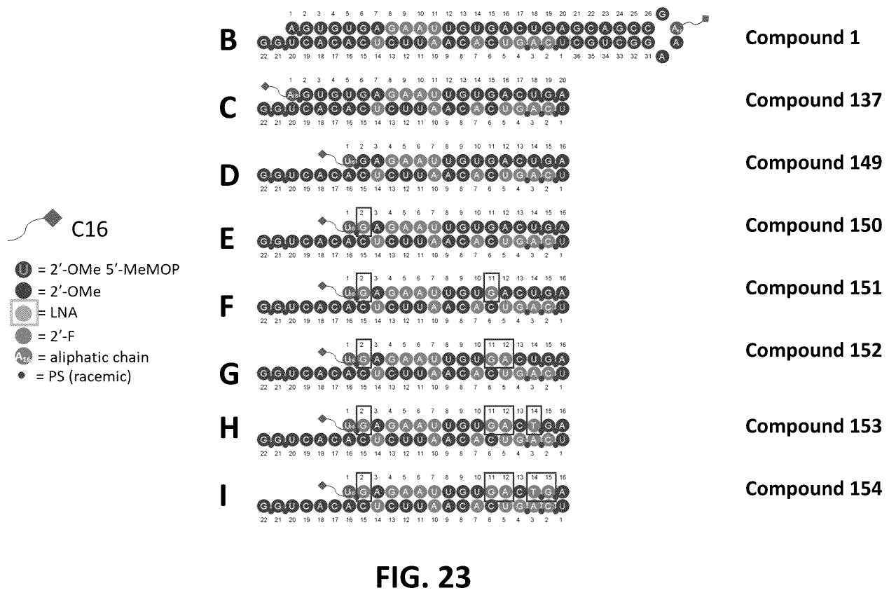

Oligonucleotide-lipid conjugates that were evaluated included those having the structures of Compounds 149-154 as shown in FIG. 23, compared to the parent oligonucleotide-lipid conjugate evaluated in previous studies (Compounds 1 and 137, described above). Each of Compounds 149-154 contain a blunt-end at the 3′ terminus of the sense strand, and a truncation at the 5′ terminus of the sense strand resulting in a 4-nt truncation (i.e., a 6-nt overhang of the antisense strand at the 3′ terminus), and a C16 lipid conjugate a the 5′ terminal of the sense strand. Each of compounds 149-154 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. The compounds comprise different positions of locked nucleic acids as further described below:
(i) Compounds 1, 137, and 149 contain no locked nucleic acids.
(ii) Compound 150 contains an LNA at position 2 of the sense strand.
(iii) Compound 151 contains an LNA at positions 2 and 11 of the sense strand.
(iv) Compound 152 contains an LNA at positions 2, 11, and 12 of the sense strand.
(v) Compound 153 contains an LNA at positions 2, 11, 12, and 14 of the sense strand.
(vi) Compound 154 contains an LNA at positions 2, 11, 12, 14, and 15 of the sense strand.

To evaluate the RNAi oligonucleotide-lipid conjugates, 6 to 10 week old C57BL6 female mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 15. Group A were control mice that received artificial cerebrospinal fluid (aCSF) only.

TABLE 15
Lipid-conjugated RNAi oligonucleotides
administered to animal groups A-I

| Group | Compound | Sense SEQ ID NOS | | Antisense SEQ ID NOs | |
|---|---|---|---|---|---|
| # | ID | Unmod | Mod | Unmod | Mod |
| A | aCSF | | | | |
| B | 1 | 310 | 338 | 324 | 352 |
| C | 137 | 372 | 386 | 324 | 352 |
| D | 149 | 549 | 551 | 324 | 352 |
| E | 150 | 549 | 552 | 324 | 352 |
| F | 151 | 549 | 553 | 324 | 352 |
| G | 152 | 549 | 554 | 324 | 352 |
| H | 153 | 550 | 555 | 324 | 352 |
| I | 154 | 551 | 556 | 324 | 352 |

Target knockdown was assessed 28 days post-injection. RNA was extracted from various tissues and murine TUBB3 mRNA levels were determined as described in Example 2.

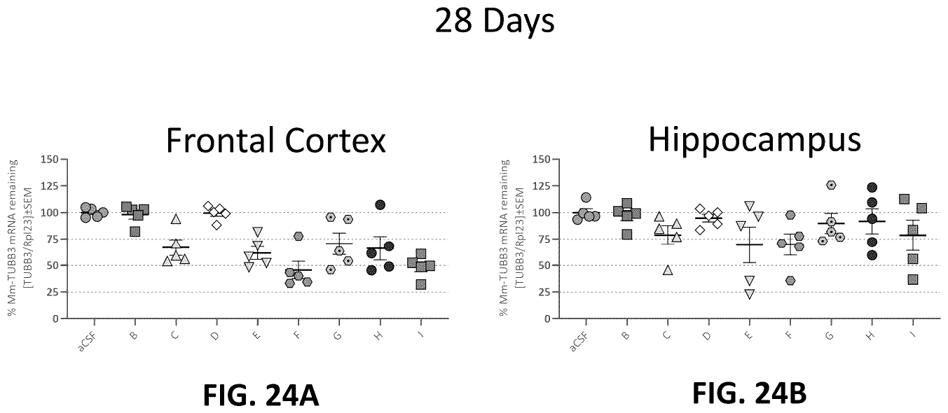

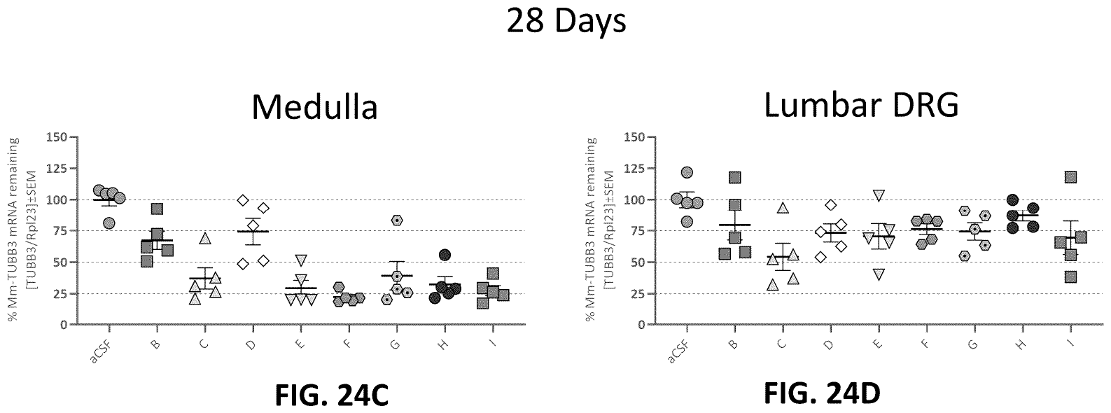

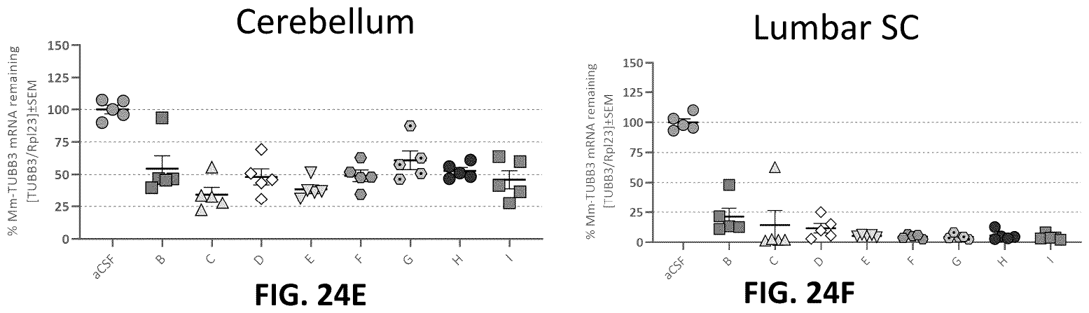

Percent TUBB3 mRNA expression in the frontal cortex, hippocampus, medulla, lumbar dorsal root ganglion, cerebellum, and lumbar spinal cord as measured for each treatment group is respectively shown in FIGS. 24A-24F. Silencing of TUBB3 mRNA expression was observed in deeper (e.g., frontal cortex and hippocampus) tissue in compounds comprising an LNA. Increased knockdown was observed with the addition of 2-3 LNAs.

### Example 16: 5′C16 Lipid Conjugated P-8 Truncated Sense Strand with Locked Nucleic Acids for Inhibition of a Neuronal Target Gene in the CNS

RNAi oligonucleotide-lipid conjugates were evaluated to determine the effect of blunt-end passenger truncations with locked nucleic acids on the guide strand in neurons. Each oligonucleotide-lipid conjugate tested comprised an antisense strand having a region of complementarity to mRNA encoding TUBB3.

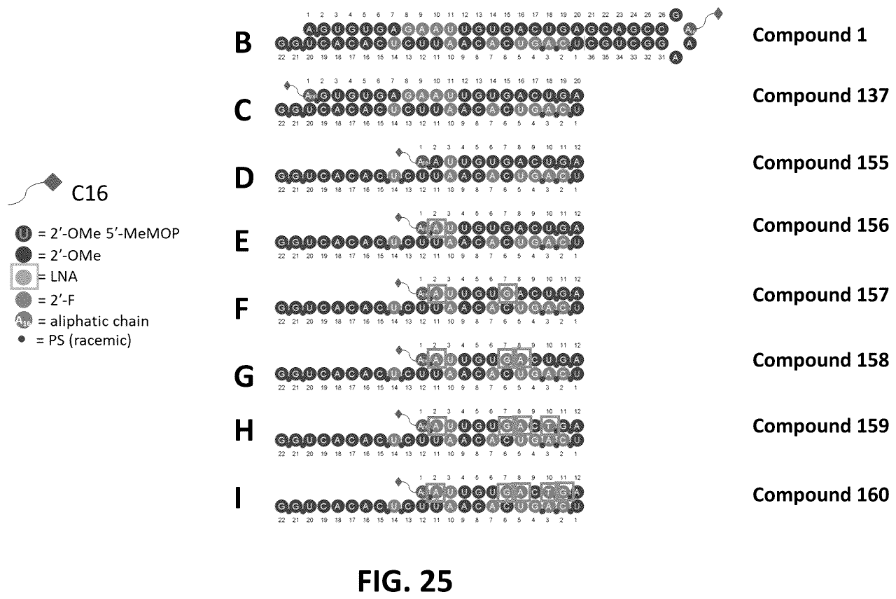

Oligonucleotide-lipid conjugates that were evaluated included those having the structures of Compounds 155-160 as shown in FIG. 25, compared to the parent oligonucleotide-lipid conjugate evaluated in previous studies (Compounds 1 and 137, described above). Each of Compounds 155-160 contain a blunt-end at the 3′ terminus of the sense strand, and a truncation at the 5′ terminus of the sense strand resulting in an 8-nt truncation (i.e., a 10-nt overhang of the antisense strand at the 3′ terminus), and a C16 lipid conjugate a the 5′ terminal of the sense strand. Additionally, Compounds 155-160 comprises two phosphorothioate linkages flanking the nucleotide at position 14 (from 5′ to 3′) of the antisense strand (i.e., phosphorothioate linkages between positions 13 and 14, and 14 and 15, of the antisense strand). Each of Compounds 155-160 comprise phosphorothioate linkages between positions: 1 and 2, 10 and 11, and 11 and 12, of the sense strand; 1 and 2, 2 and 3, 3 and 4, 13 and 14, 14 and 15, 20 and 21, and 21 and 22, of the antisense strand. The compounds comprise different positions of locked nucleic acids as further described below:
(i) Compounds 1, 137, and 155 contain no locked nucleic acids.
(ii) Compound 156 contains an LNA at position 2 of the sense strand.
(iii) Compound 157 contains an LNA at positions 2 and 7 of the sense strand.
(iv) Compound 152 contains an LNA at positions 2, 7, and 8 of the sense strand.
(v) Compound 153 contains an LNA at positions 2, 7, 8, and 10 of the sense strand.
(vi) Compound 154 contains an LNA at positions 2, 7, 8, 10, and 11 of the sense strand.

To evaluate the RNAi oligonucleotide-lipid conjugates, 6 to 10 week old C57BL6 female mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 16. Group A were control mice that received artificial cerebrospinal fluid (aCSF) only.

TABLE 16
Lipid-conjugated RNAi oligonucleotides
administered to animal groups A-I

| Group | Compound | Sense SEQ ID NOS | | Antisense SEQ ID NOs | |
|---|---|---|---|---|---|
| # | ID | Unmod | Mod | Unmod | Mod |
| A | aCSF | | | | |
| B | 1 | 310 | 338 | 324 | 352 |
| C | 137 | 372 | 386 | 324 | 352 |
| D | 155 | 557 | 559 | 324 | 533 |
| E | 156 | 557 | 560 | 324 | 533 |
| F | 157 | 557 | 561 | 324 | 533 |
| G | 158 | 557 | 562 | 324 | 533 |
| H | 159 | 558 | 563 | 324 | 533 |
| I | 160 | 558 | 564 | 324 | 533 |

Target knockdown was assessed 28 days post-injection. RNA was extracted from various tissues and murine TUBB3 mRNA levels were determined as described in Example 2.

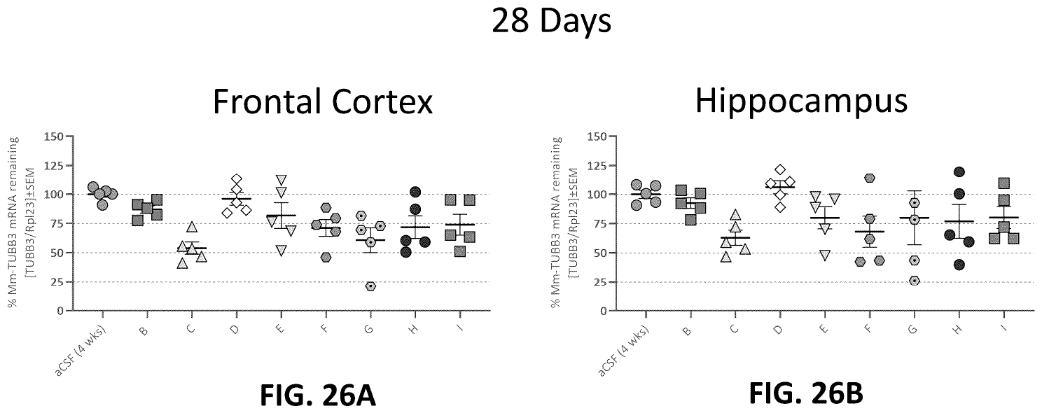

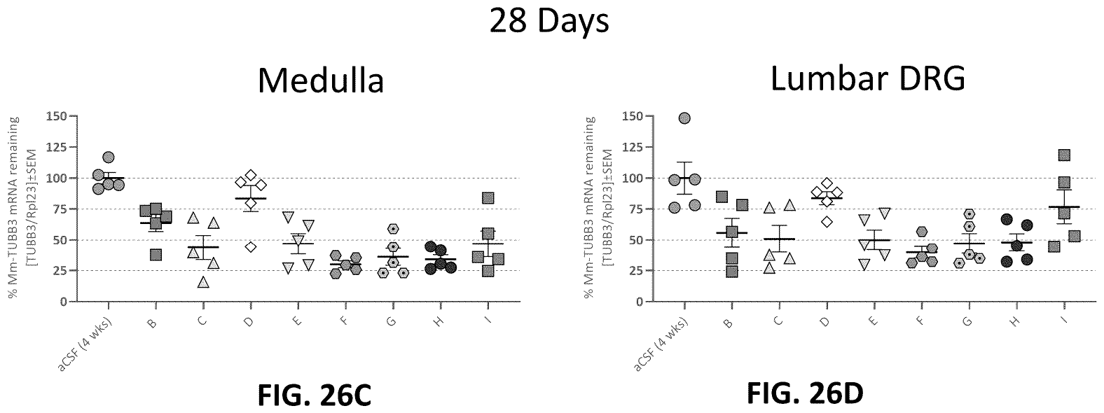

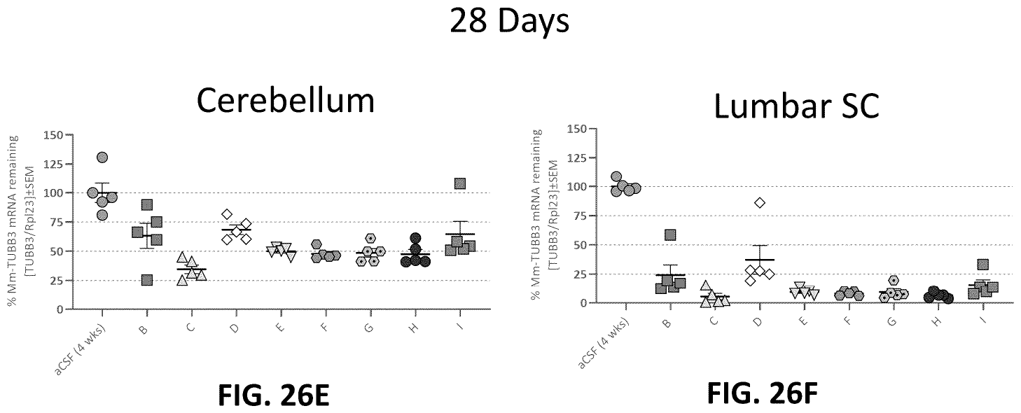

Percent TUBB3 mRNA expression in the frontal cortex, hippocampus, medulla, lumbar dorsal root ganglion, cerebellum, and lumbar spinal cord as measured for each treatment group is respectively shown in FIGS. 26A-26F. Inclusion of at least one LNA was beneficial for neuronal knockdown in molecules with a P-8 truncation (compare Compound 155 to Compounds 156-160). Silencing of TUBB3 mRNA expression was observed in deeper (e.g., frontal cortex and hippocampus) tissue in compounds comprising an LNA. Increased knockdown was observed with the addition of 2-3 LNAs.

### Example 17: 5′ P-6 Truncations with Locked Nucleic Acids on the Sense Strand for Inhibition of an Oligodendrocyte Target Gene in the CNS

RNAi oligonucleotide-lipid conjugates were evaluated to determine the effect of blunt-end passenger truncations with locked nucleic acids on the exposed guide strand in oligodendrocytes. Each oligonucleotide-lipid conjugate tested comprised an antisense strand having a region of complementarity to mRNA encoding UGT8, which is a protein expressed in oligodendrocytes of the CNS.

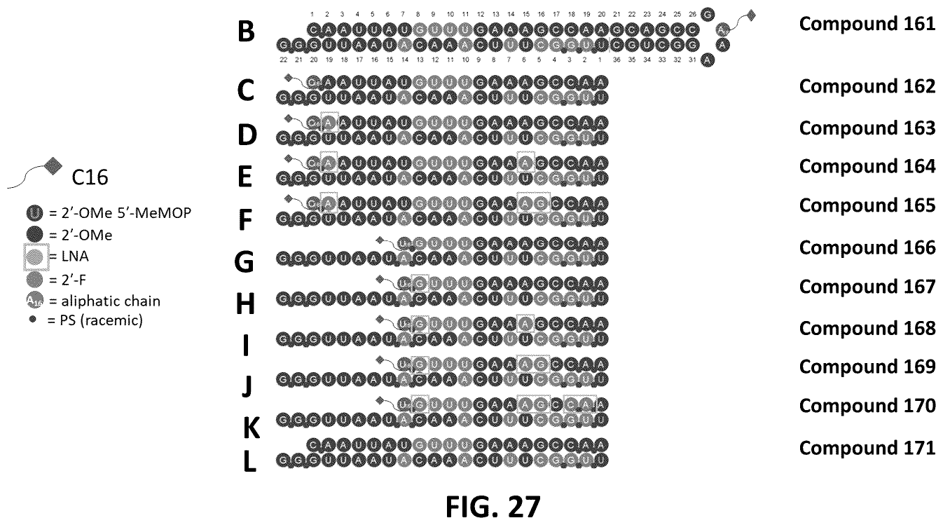

Oligonucleotide-lipid conjugates that were evaluated included those having the structures of Compounds 163-171 as shown in FIG. 27, compared to the parent oligonucleotide-lipid conjugates of Compounds 161 (tetraloop oligonucleotide with C16 lipid conjugated at second nucleotide of tetraloop) and 162 (blunt-end oligonucleotide with C16 lipid conjugated at 5′ terminal nucleotide). Each of Compounds 166-170 contain a blunt-end at the 3′ terminus of the sense strand, a truncation at the 5′ terminus of the sense strand resulting in a 6-nt truncation (i.e. an 8-nt antisense strand overhang at the 3′ terminus), and a C16 lipid conjugate a the 5′ terminal of the sense strand. Each of Compounds 162-165 contain a blunt-end at the 3′ terminus of the sense strand, and a C16 lipid conjugate a the 5′ terminal of the sense strand. Compound 171 contains a blunt-end at the 3′ terminus of the sense strand and does not comprise a lipid conjugate a the 5′ terminal of the sense strand. Compounds 162-165 and 177 comprise phosphorothioate linkages between positions: 1 and 2, 18 and 19, and 19 and 20, of the sense strand; 1 and 2, 2 and 3, 3 and 4, 20 and 21, and 21 and 22, of the antisense strand. Compounds 166-170 comprise phosphorothioate linkages between positions: 1 and 2, 18 and 19, and 19 and 20, of the sense strand; 1 and 2, 2 and 3, 3 and 4, 13 and 14, 14 and 15, 20 and 21, and 21 and 22, of the antisense strand. The compounds comprise different positions of locked nucleic acids as further described below:
(i) Compounds 161, 162, 166, and 171 contain no locked nucleic acids.
(ii) Compounds 163 and 167 contain an LNA at position 2 of the sense strand.
(iii) Compound 164 contains LNAs at positions 2 and 15.
(iv) Compound 165 contains LNAs at positions 2, 15, and 16.
(v) Compound 168 contains LNAs at positions 2 and 9.
(vi) Compound 169 contains LNAs at positions 2, 9, and 10.
(vii) Compound 170 contains LNAs at positions 2, 9, 10, 12, and 13.

To evaluate the RNAi oligonucleotide-lipid conjugates, 6 to 10 week old C57BL6 female mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 17. Group A were control mice that received artificial cerebrospinal fluid (aCSF) only.

TABLE 17
Lipid-conjugated RNAi oligonucleotides
administered to animal groups A-L

| Group | Compound | Sense SEQ ID NOS | | Antisense SEQ ID NOs | |
|---|---|---|---|---|---|
| # | ID | Unmod | Mod | Unmod | Mod |
| A | aCSF | | | | |
| B | 161 | 580 | 581 | 577 | 578 |
| C | 162 | 565 | 567 | 577 | 578 |
| D | 163 | 565 | 568 | 577 | 578 |
| E | 164 | 565 | 569 | 577 | 578 |
| F | 165 | 565 | 570 | 577 | 578 |
| G | 166 | 566 | 571 | 577 | 579 |
| H | 167 | 566 | 572 | 577 | 579 |
| I | 168 | 566 | 573 | 577 | 579 |
| J | 169 | 566 | 574 | 577 | 579 |
| K | 170 | 566 | 575 | 577 | 579 |
| L | 171 | 565 | 576 | 577 | 578 |

Target knockdown was assessed 28 days post-injection. RNA was extracted from CNS tissue and murine UGT8 mRNA levels were determined by qPCR (normalized to endogenous housekeeping gene Rpl23). The percentage of murine mRNA remaining in the samples from treated mice was determined using the 2-ΔΔCt (“delta-delta Ct”) method (Livak and Schmittgen (2001) METHODS 25:402-408).

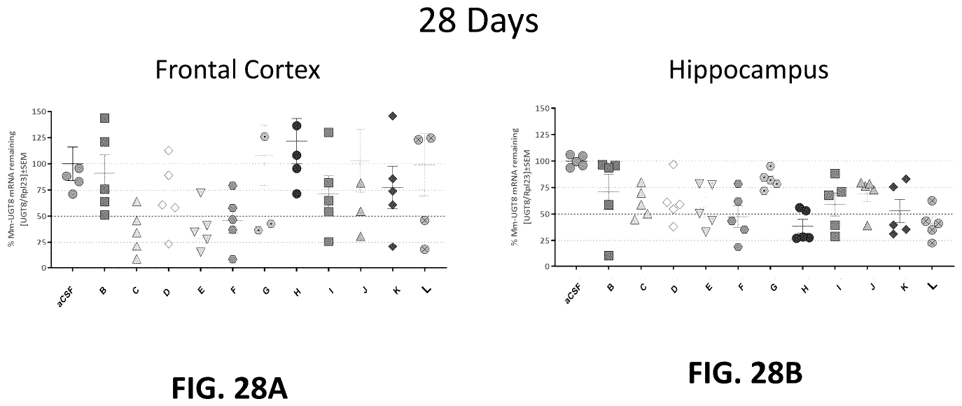

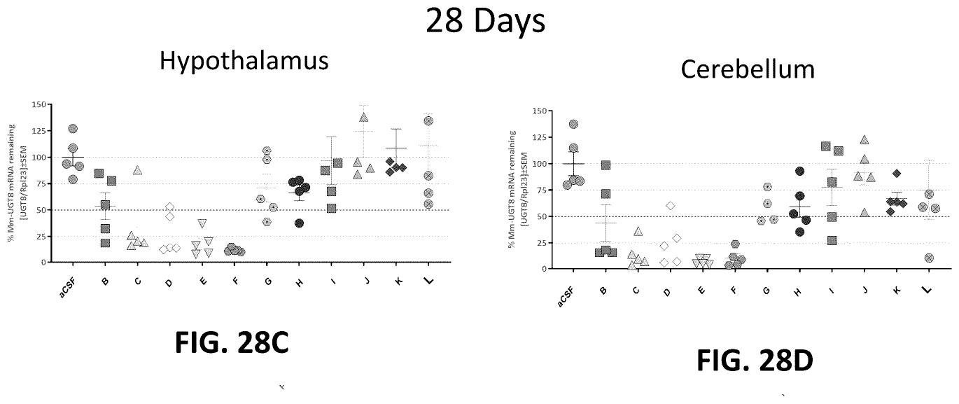

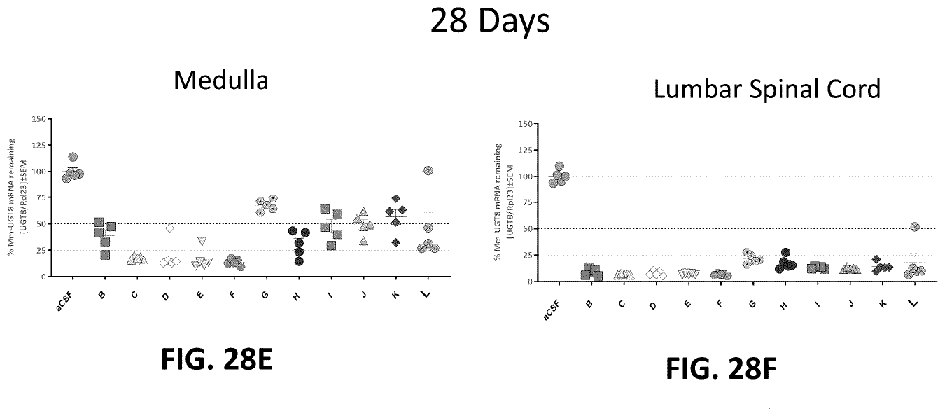

Percent UGT8 mRNA expression in the frontal cortex, hippocampus, hypothalamus, cerebellum, medulla, and lumbar spinal cord as measured for each treatment group is respectively shown in FIGS. 28A-28F. Silencing of UGT8 mRNA expression was observed in all tissues evaluated for most treatment groups. Inclusion of a P-6 truncation decreased activity independent of the number of LNAs present. Lipid conjugation increased knockdown in deep (e.g., frontal cortex and hypothalamus) tissue (compare Compound 171 to Compounds 162-165).

### Example 18: 5′ P-6 Truncations with Locked Nucleic Acids on the Sense Strand for Inhibition in an Astrocyte Target Gene in the CNS

RNAi oligonucleotide-lipid conjugates were evaluated to determine the effect of blunt-end passenger truncations with locked nucleic acids on the exposed guide strand in neurons. Each oligonucleotide-lipid conjugate tested comprised an antisense strand having a region of complementarity to mRNA encoding GFAP, which is a protein expressed in astrocytes of the central nervous system (CNS).

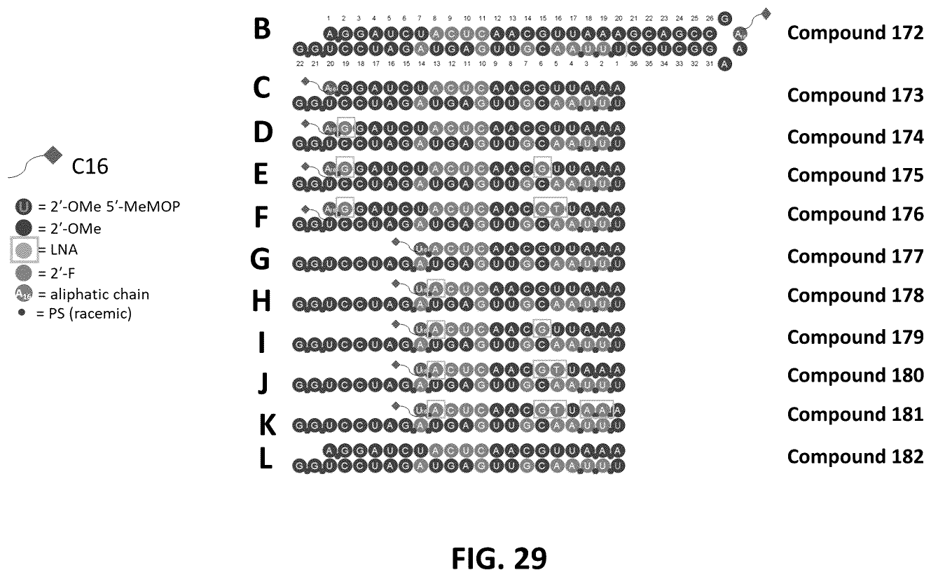

Oligonucleotide-lipid conjugates that were evaluated included those having the structures of Compounds 174-182 as shown in FIG. 29, compared to the parent oligonucleotide-lipid conjugates of Compounds 172 (tetraloop oligonucleotide with C16 lipid conjugated at second nucleotide of tetraloop) and 173 (blunt-end oligonucleotide with C16 lipid conjugated at 5′ terminal nucleotide). Each of Compounds 177-181 contain a blunt-end at the 3′ terminus of the sense strand, a truncation at the 5′ terminus of the sense strand resulting in a 6-nt truncation (i.e. an 8-nt antisense strand overhang at the 3′ terminus), and a C16 lipid conjugate a the 5′ terminal of the sense strand. Each of Compounds 173-176 contain a blunt-end at the 3′ terminus of the sense strand, and a C16 lipid conjugate a the 5′ terminal of the sense strand. Compound 182 contains a blunt-end at the 3′ terminus of the sense strand and does not comprise a lipid conjugate a the 5′ terminal of the sense strand. Compounds 173-176 and 182 comprise phosphorothioate linkages between positions: 1 and 2, 18 and 19, and 19 and 20, of the sense strand; 1 and 2, 2 and 3, 3 and 4, 20 and 21, and 21 and 22, of the antisense strand. Compounds 177-181 comprise phosphorothioate linkages between positions: 1 and 2, 18 and 19, and 19 and 20, of the sense strand; 1 and 2, 2 and 3, 3 and 4, 13 and 14, 14 and 15, 20 and 21, and 21 and 22, of the antisense strand. The compounds comprise different positions of locked nucleic acids as further described below:
(i) Compounds 172, 173, 177, and 182 contain no locked nucleic acids.
(ii) Compounds 174 and 178 contain an LNA at position 2 of the sense strand.
(iii) Compound 175 contains LNAs at positions 2 and 15.
(iv) Compound 176 contains LNAs at positions 2, 15, and 16.
(v) Compound 179 contains LNAs at positions 2 and 9.
(vi) Compound 180 contains LNAs at positions 2, 9, and 10.
(vii) Compound 181 contains LNAs at positions 2, 9, 10, 12, and 13.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 18. Group A were control mice that received artificial cerebrospinal fluid (aCSF) only.

TABLE 18
Lipid-conjugated RNAi oligonucleotides
administered to animal groups A-L

| | | Sense | | Antisense | |
|---|---|---|---|---|---|
| Group | Compound | SEQ ID NOS | | SEQ ID NOs | |
| # | ID | Unmod | Mod | Unmod | Mod |
| A | aCSF | | | | |
| B | 172 | 582 | 585 | 596 | 597 |
| C | 173 | 583 | 586 | 596 | 597 |
| D | 174 | 583 | 587 | 596 | 597 |
| E | 175 | 583 | 588 | 596 | 597 |
| F | 176 | 583 | 589 | 596 | 597 |
| G | 177 | 584 | 590 | 596 | 598 |
| H | 178 | 584 | 591 | 596 | 598 |
| I | 179 | 584 | 592 | 596 | 598 |
| J | 180 | 620 | 593 | 596 | 598 |
| K | 181 | 620 | 594 | 596 | 598 |
| L | 182 | 583 | 595 | 596 | 597 |

Target knockdown was assessed 28 days post-injection. RNA was extracted from CNS tissue and murine GFAP mRNA levels were determined by qPCR (normalized to endogenous housekeeping gene Rpl23). The percentage of murine mRNA remaining in the samples from treated mice was determined using the 2-ΔΔCt (“delta-delta Ct”) method (Livak and Schmittgen (2001) METHODS 25:402-408).

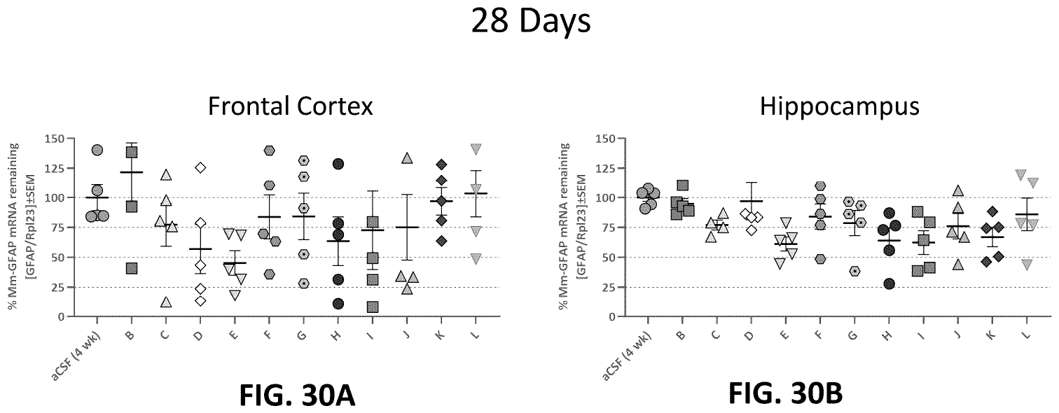

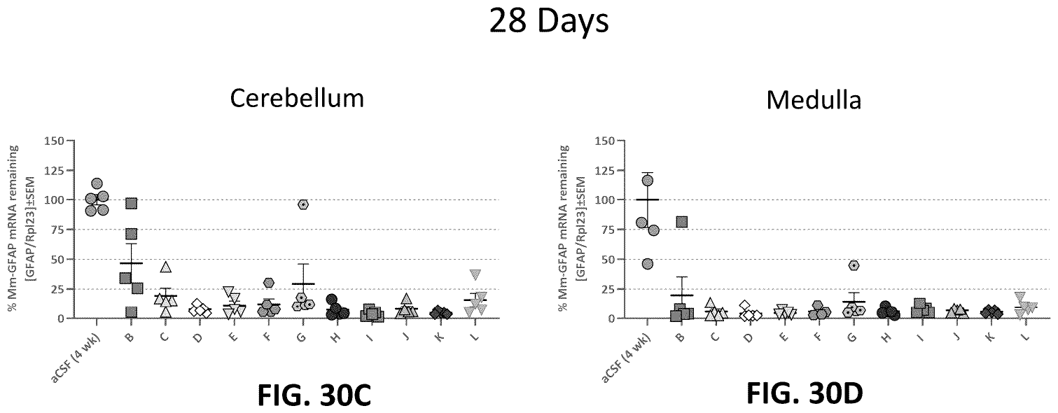

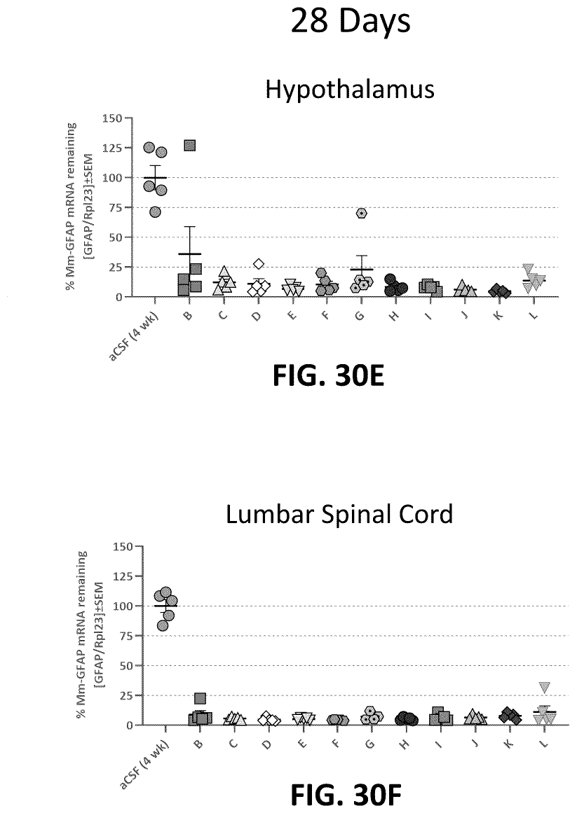

Percent GFAP mRNA expression in the frontal cortex, hippocampus, cerebellum, medulla, lumbar dorsal root ganglion, hypothalamus, and lumbar spinal cord as measured for each treatment group is respectively shown in FIGS. 30A-30F. Silencing of GFAP mRNA expression was observed in all tissues evaluated for most treatment groups. Oligonucleotides comprising a P-6 truncation demonstrated equivalent knockdown to full length oligonucleotides regardless of the presences of LNAs.

Overall, the data in Examples 13, 16, and 17 demonstrate oligonucleotide chemistries for cell-type specific targeting. For example, full length blunt structures with a lipid conjugate can be used to have enhanced targeting in astrocytes and oligodendrocytes with some potency for neurons. If neurons and/or astrocytes but not oligodendrocytes are of interest to target, oligonucleotides comprising a P-6 truncation of a sense strand comprising at least one LNA can be used. Additionally, use of a full length blunt-end oligonucleotide can target oligodendrocytes and astrocytes, but may also target neurons. To target astrocytes over oligodendrocytes, a P-6 sense strand truncated oligonucleotide can be used. Furthermore, if targeting oligodendrocytes and astrocytes with the exclusion of neurons is of interest an oligonucleotide comprising a C16 lipid on a tetraloop can be used. Together, these examples describe specific oligonucleotide structures for targeting or excluding target of specific cell types in the CNS.

### Example 19: Truncated Oligonucleotide Positional Lipid Walk on the Sense Strand for Inhibition of a Neuronal Target Gene in the CNS

RNAi oligonucleotide-lipid conjugates were evaluated to determine the effect of lipid position on truncated blunt-end oligonucleotides. Each oligonucleotide-lipid conjugate tested comprised an antisense strand having a region of complementarity to mRNA encoding TUBB3.

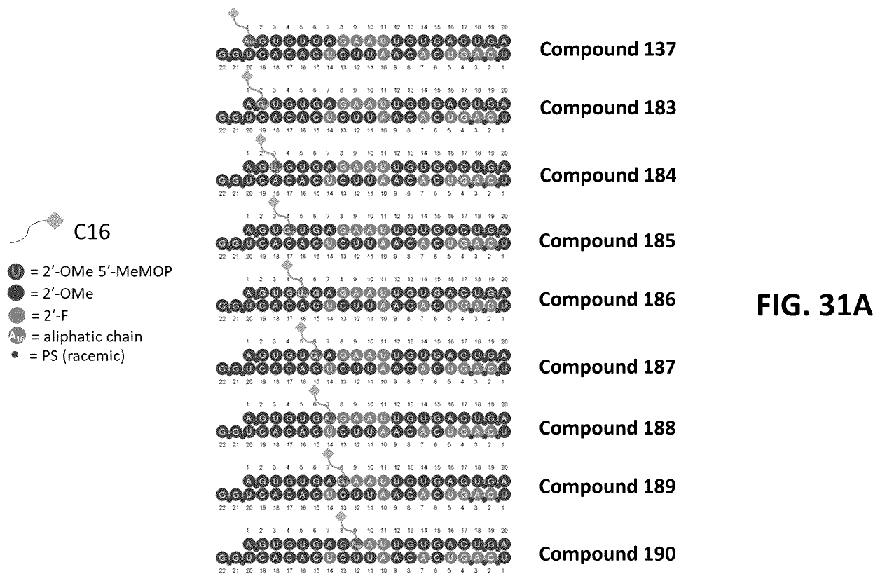

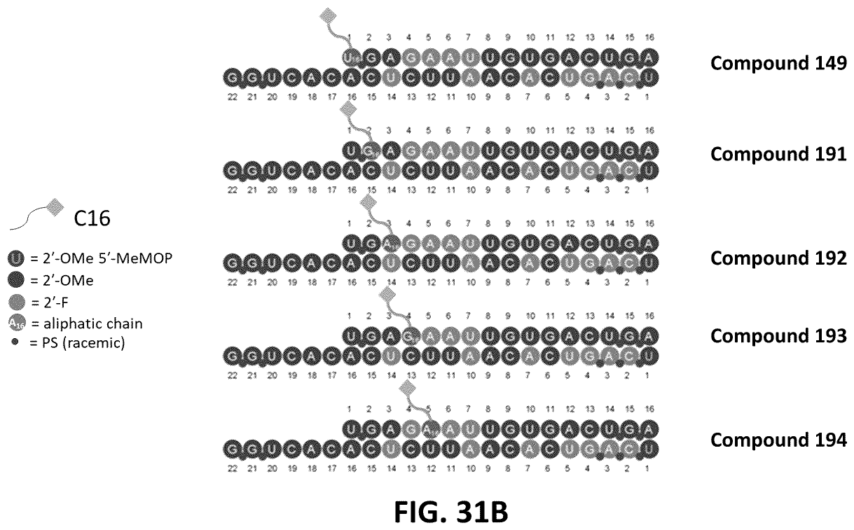

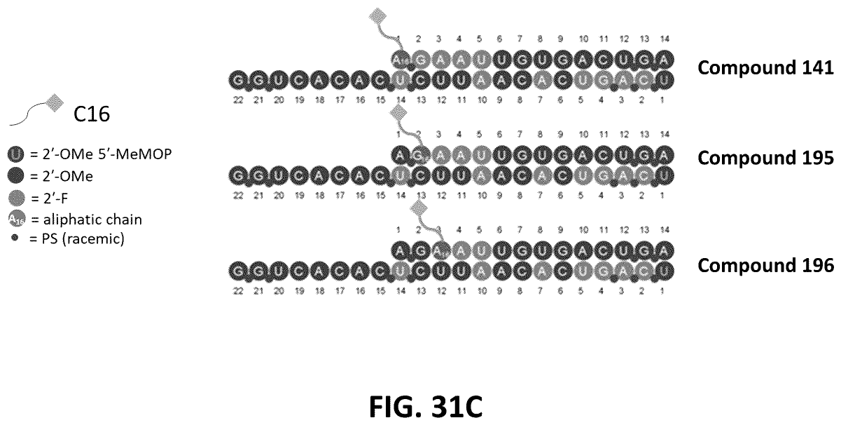

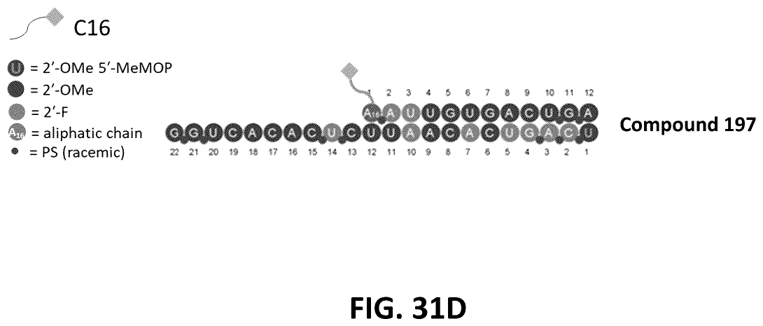

Oligonucleotide-lipid conjugates that were evaluated included those having the structures of Compounds 183-197 as shown in FIGS. 31A-31D, compared to the parent oligonucleotide-lipid conjugates of Compounds 137, 149, and 141 (blunt-end oligonucleotides comprising a 2, 6, and 8 nucleotide antisense strand overhang, respectively, as described above). Each of Compounds 183-190 contain a blunt-end at the 3′ terminus of the sense strand, and a 2-nt antisense strand overhang at the 3′ terminus. Each of Compounds 191-194 contain a blunt-end at the 3′ terminus of the sense strand, and a 4-nt truncation at the 5′ terminus of the sense strand (i.e. a 6-nt antisense strand overhang at the 3′ terminus). Compounds 195 and 196 contain a blunt-end at the 3′ terminus of the sense strand, and a 6-nt truncation at the 5′ terminus of the sense strand (i.e. an 8-nt antisense strand overhang at the 3′ terminus). Compound 197 contains a blunt-end at the 3′ terminus of the sense strand, and a an 8-nt truncation at the 5′ terminus of the sense strand (i.e. a 10-nt antisense strand overhang at the 3′ terminus).

Compounds 183-190 comprise phosphorothioate linkages between positions: 1 and 2, 18 and 19, and 19 and 20, of the sense strand; 1 and 2, 2 and 3, 3 and 4, 20 and 21, and 21 and 22, of the antisense strand. Compounds 191-194 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. Compounds 195 and 196 comprise phosphorothioate linkages between positions: 1 and 2, 12 and 13, and 13 and 14, of the sense strand; 1 and 2, 2 and 3, 3 and 4, 13 and 14, 14 and 15, 20 and 21, and 21 and 22, of the antisense strand. Compound 197 comprises phosphorothioate linkages between positions: 1 and 2, 10 and 11, and 11 and 12, of the sense strand; 1 and 2, 2 and 3, 3 and 4, 13 and 14, 14 and 15, 20 and 21, and 21 and 22, of the antisense strand.

The compounds comprise a C16 lipid conjugated at a different position of the sense strand as further described below:
(i) Compounds 137, 149, 141, and 197 comprise a C16 lipid at position 1.
(ii) Compounds 183, 191, and 195 comprise a C16 lipid at position 2.
(iii) Compounds 184, 192, and 196 comprise a C16 lipid at position 3.
(iv) Compounds 185 and 193 comprise a C16 lipid at position 4.
(v) Compounds 186 and 194 comprise a C16 lipid at position 5.
(vi) Compound 187 comprises a C16 lipid at position 6.
(vii) Compound 188 comprises a C16 lipid at position 7.
(viii) Compound 189 comprises a C16 lipid at position 8.
(ix) Compound 190 comprises a C16 lipid at position 9.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 19. Group A were control mice that received artificial cerebrospinal fluid (aCSF) only.

TABLE 19
Lipid-conjugated RNAi oligonucleotides administered to animals

| Compound | Sense SEQ ID NOS | | Antisense SEQ ID NOs | |
|---|---|---|---|---|
| ID | Unmod | Mod | Unmod | Mod |
| aCSF | — | — | — | — |
| 137 | 372 | 386 | 324 | 352 |
| 183 | 372 | 599 | 324 | 352 |
| 184 | 372 | 600 | 324 | 352 |
| 185 | 372 | 601 | 324 | 352 |
| 186 | 372 | 602 | 324 | 352 |
| 187 | 372 | 603 | 324 | 352 |
| 188 | 372 | 604 | 324 | 352 |
| 189 | 372 | 605 | 324 | 352 |
| 190 | 372 | 606 | 324 | 352 |
| 149 | 549 | 551 | 324 | 352 |
| 191 | 549 | 607 | 324 | 352 |
| 192 | 549 | 608 | 324 | 352 |
| 193 | 549 | 608 | 324 | 352 |
| 194 | 549 | 610 | 324 | 352 |
| 141 | 537 | 542 | 324 | 533 |
| 195 | 537 | 611 | 324 | 533 |
| 196 | 537 | 612 | 324 | 533 |
| 197 | 557 | 613 | 324 | 533 |

Target knockdown was assessed 28 days post-injection. RNA was extracted from CNS tissue and murine TUBB3 mRNA levels were determined as described in Example 2.

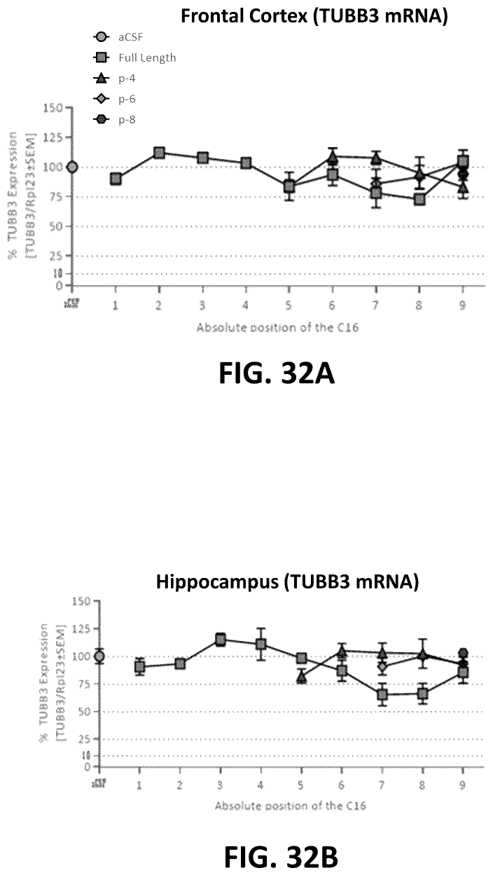

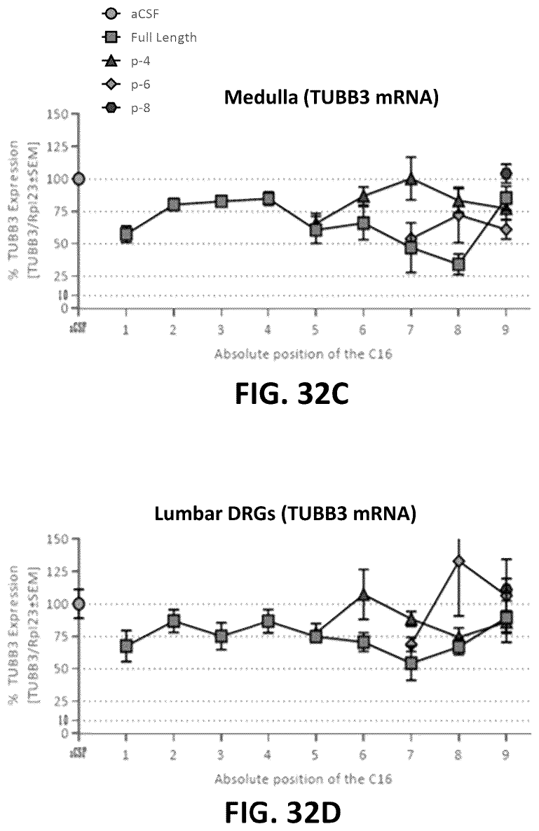

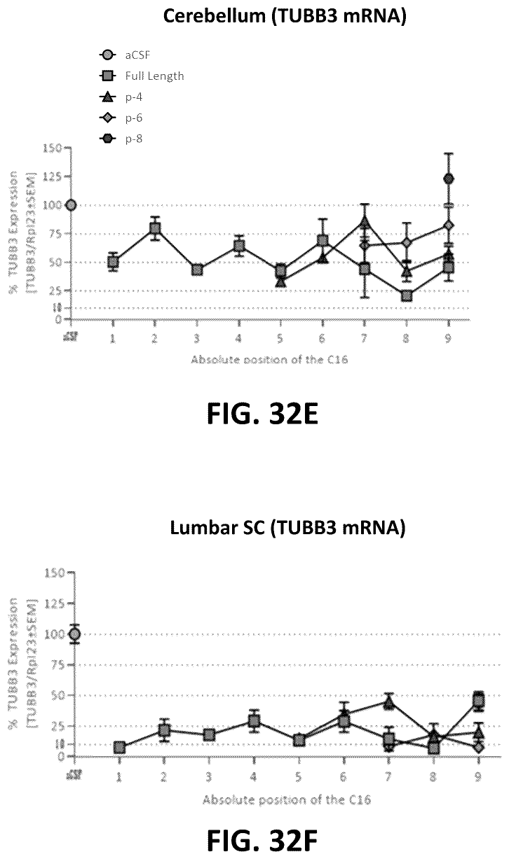

Percent TUBB3 mRNA expression in the frontal cortex, hippocampus, medulla, lumbar dorsal root ganglion, cerebellum, and lumbar spinal cord as measured for each treatment group is respectively shown in FIGS. 32A-32F. Silencing of TUBB3 mRNA expression was observed in all tissues evaluated for several treatment groups. All structures comprising a 5′ terminal lipid (position 1) enhanced neuronal knockdown in each tissue of the CNS.

### Example 20: Truncated Sense Strand Oligonucleotides with 5′ and 3′ Overhangs for Inhibition of an Astrocyte Target Gene in the CNS

RNAi oligonucleotide-lipid conjugates were evaluated to determine the effect of the length of 5′ and/or 3′ truncations on the sense strand of an oligonucleotide. Each oligonucleotide-lipid conjugate tested comprised an antisense strand having a region of complementarity to mRNA encoding GFAP.

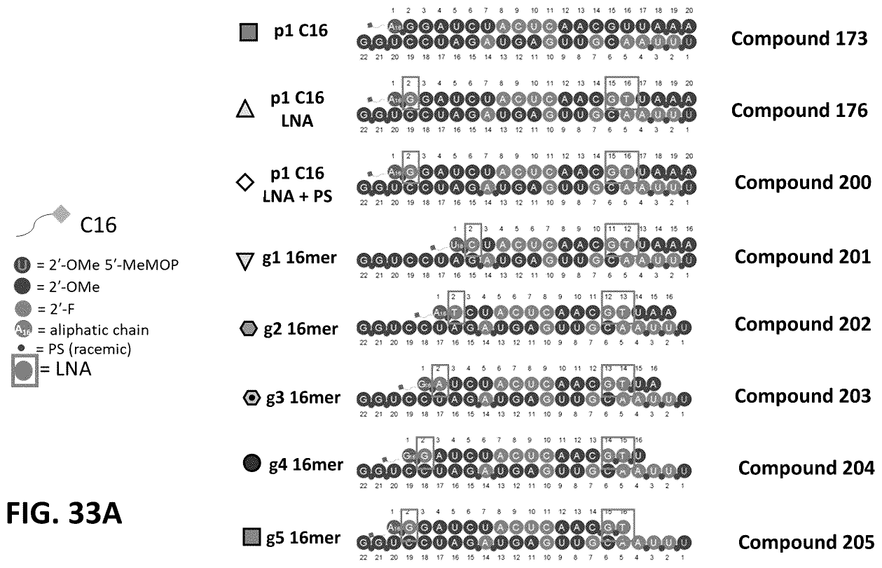

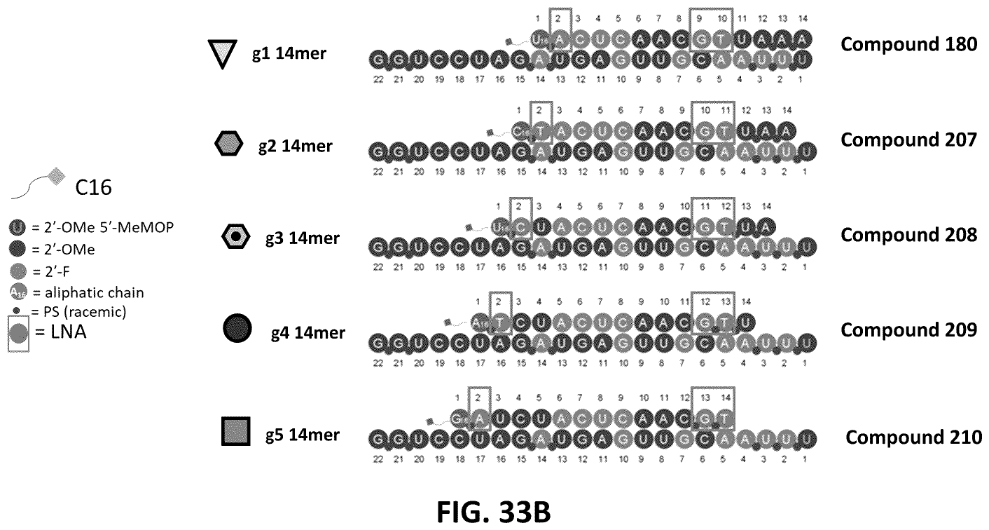

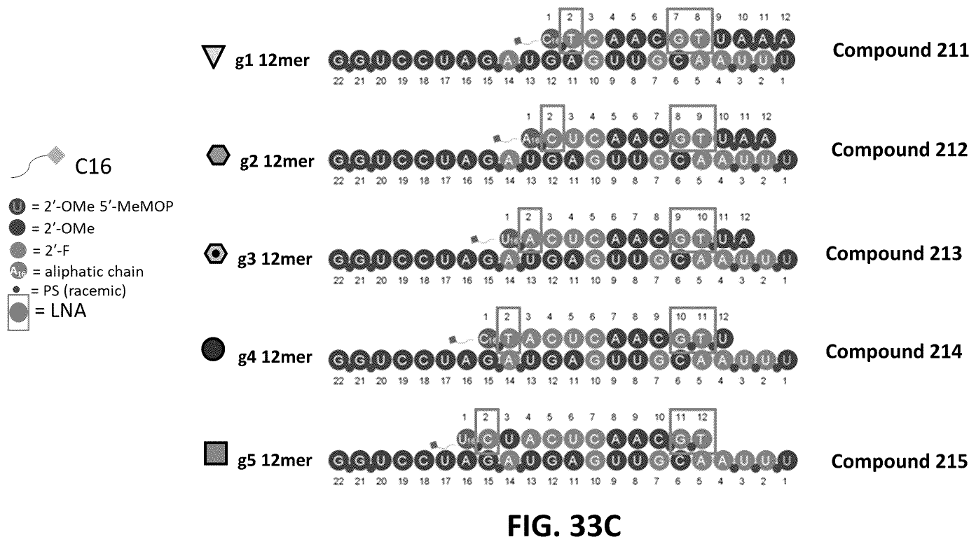

Oligonucleotide-lipid conjugates that were evaluated included those having the structures of Compounds 180, 201-205 and 207-215 as shown in FIGS. 33A-33C, compared to the parent oligonucleotide-lipid conjugates of Compounds 173, 176, and 200. Each of Compounds 173, 176, and 200 contain a blunt end at the 3′ terminus of the sense strand, and a 2-nt overhang at the 3′ terminus of the antisense strand. Compounds 201-205 comprise a sense strand 16 nucleotides in length. Compounds 180 and 207-210 comprise a sense strand 14 nucleotides in length. Compounds 211-215 comprise a sense strand 12 nucleotides in length.

Compounds 176, 180, 200-205, and 207-215 comprise locked nucleic acids on the sense strand at the nucleotides complementary to nucleotides 5 and 6 of the antisense strand. Compounds 176, 180, 200-205, and 207-215 comprise a locked nucleic acid on position 2 of the sense strand.

Compounds 173, 176, and 200 comprise phosphorothioate linkages between positions: 1 and 2, 18 and 19, and 19 and 20 of the sense strand; 1 and 2, 2 and 3, 3 and 4, 20 and 21, and 21 and 22, of the antisense strand. Compound 200 comprises additional phosphorothioate linkages between positions 13 and 14 and 14 and 15 of the antisense strand. Compounds 201-205 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. Compounds 180 and 207-210 comprise phosphorothioate linkages between positions: 1 and 2, 12 and 13, and 13 and 14, of the sense strand; 1 and 2, 2 and 3, 3 and 4, 13 and 14, 14 and 15, 20 and 21, and 21 and 22, of the antisense strand. Compounds 211-215 comprise phosphorothioate linkages between positions: 1 and 2, 10 and 11, and 11 and 12, of the sense strand; 1 and 2, 2 and 3, 3 and 4, 13 and 14, 14 and 15, 20 and 21, and 21 and 22, of the antisense strand.

Compounds 173, 176, 180, 200-205, and 207-215 comprise a C16 lipid conjugated to position 1 of the sense strand.

The compounds comprise different length 5′ and 3′ overhangs of the antisense strand as further described below:
(i) Compounds 173, 176, and 200 comprise a 2-nt antisense strand overhang at the 3′ terminus.
(ii) Compound 201 comprises a 6-nt antisense strand overhang at the 3′ terminus.
(iii) Compound 202 comprises a 5-nt antisense strand overhang at the 3′ terminus and a 1-nt antisense strand overhang at the 5′ terminus.
(iv) Compound 203 comprises a 4-nt antisense strand overhang at the 3′ terminus and a 2-nt antisense strand overhang at the 5′ terminus.
(v) Compound 204 comprises a 3-nt antisense strand overhang at the 3′ terminus and a 3-nt antisense strand overhang at the 5′ terminus.
(vi) Compound 205 comprises a 2-nt antisense strand overhang at the 3′ terminus and a 4-nt antisense strand overhang at the 5′ terminus.
(vii) Compound 180 comprises a 8-nt antisense strand overhang at the 3′ terminus.
(viii) Compound 207 comprises a 7-nt antisense strand overhang at the 3′ terminus and a 1-nt antisense strand overhang at the 5′ terminus.
(ix) Compound 208 comprises a 6-nt antisense strand overhang at the 3′ terminus and a 2-nt antisense strand overhang at the 5′ terminus.
(x) Compound 209 comprises a 5-nt antisense strand overhang at the 3′ terminus and a 3-nt antisense strand overhang at the 5′ terminus.
(xi) Compound 210 comprises a 4-nt antisense strand overhang at the 3′ terminus and a 4-nt antisense strand overhang at the 5′ terminus.
(xii) Compound 211 comprises a 10-nt antisense strand overhang.
(xiii) Compound 212 comprises a 9-nt antisense strand overhang at the 3′ terminus and a 1-nt antisense strand overhang at the 5′ terminus.
(xiv) Compound 213 comprises a 8-nt antisense strand overhang at the 3′ terminus and a 2-nt antisense strand overhang at the 5′ terminus.
(xv) Compound 214 comprises a 7-nt antisense strand overhang at the 3′ terminus and a 3-nt antisense strand overhang at the 5′ terminus.
(xvi) Compound 215 comprises a 6-nt antisense strand overhang at the 3′ terminus and a 4-nt antisense strand overhang at the 5′ terminus.

To evaluate the RNAi oligonucleotide-lipid conjugates, 6 to 10 week old C57BL6 mice were given a single intrathecal (i.t.) injection of 100 μg oligonucleotide-lipid conjugate formulated in artificial cerebrospinal fluid (aCSF). The RNAi oligonucleotide-lipid conjugate administered to each animal group is outlined in Table 20. Control mice received artificial cerebrospinal fluid (aCSF) only.

TABLE 20
GFAP RNAi oligonucleotides administered to animals

| | | Sense | | Antisense | |
|---|---|---|---|---|---|
| | Compound | SEQ ID NOS | | SEQ ID NOS | |
| Group | ID | Unmod | Mod | Unmod | Mod |

Controls

| | | | | | |
|---|---|---|---|---|---|
| | aCSF | — | — | — | — |
| 5′C16 Blunt | 173 | 583 | 586 | 596 | 597 |
| 5′C16 Blunt + | 176 | 583 | 589 | 596 | 597 |
| LNA | | | | | |
| 5′C16 Blunt + | 200 | 614 | 589 | 596 | 598 |
| LNA + g14 2′F | | | | | |
| PS | | | | | |

16mer Sense Strands

| | | | | | |
|---|---|---|---|---|---|
| 16mer, g1 | 201 | 615 | 672 | 596 | 598 |
| 16mer, g2 | 202 | 616 | 673 | 596 | 598 |
| 16mer, g3 | 203 | 617 | 674 | 596 | 598 |
| 16mer, g4 | 204 | 618 | 675 | 596 | 598 |
| 16mer, g5 | 205 | 619 | 676 | 596 | 598 |

14mer Sense Strands

| | | | | | |
|---|---|---|---|---|---|
| 14mer, g1 | 180 | 620 | 593 | 596 | 598 |
| 14mer, g2 | 207 | 621 | 677 | 596 | 598 |
| 14mer, g3 | 208 | 622 | 678 | 596 | 598 |
| 14mer, g4 | 209 | 623 | 679 | 596 | 598 |
| 14mer, g5 | 210 | 624 | 680 | 596 | 598 |

12mer Sense Stands

| | | | | | |
|---|---|---|---|---|---|
| 12mer, g1 | 211 | 625 | 681 | 596 | 598 |
| 12mer, g2 | 212 | 626 | 682 | 596 | 598 |
| 12mer, g3 | 213 | 627 | 683 | 596 | 598 |
| 12mer, g4 | 214 | 628 | 684 | 596 | 598 |
| 12mer, g5 | 215 | 629 | 685 | 596 | 598 |

Target knockdown was assessed 28 days post-injection. RNA was extracted from CNS tissue and murine GFAP mRNA levels were determined as described in Example 17.

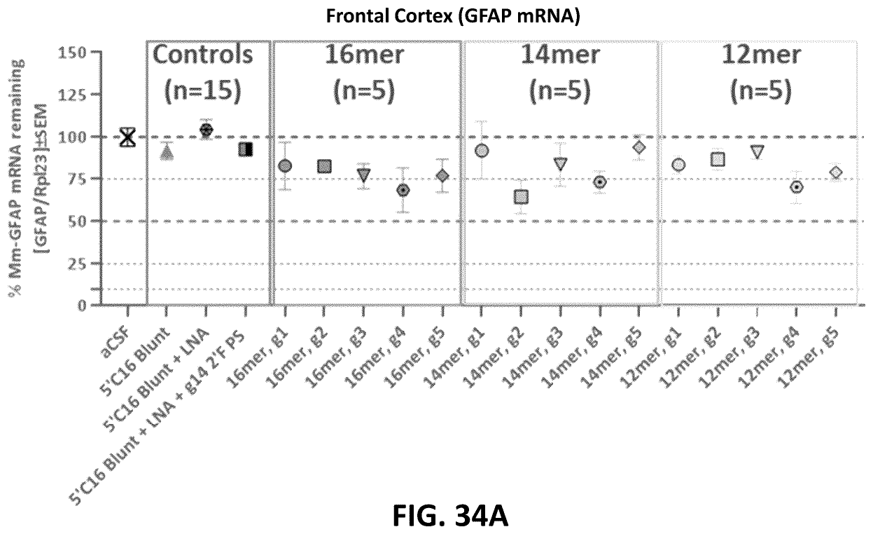

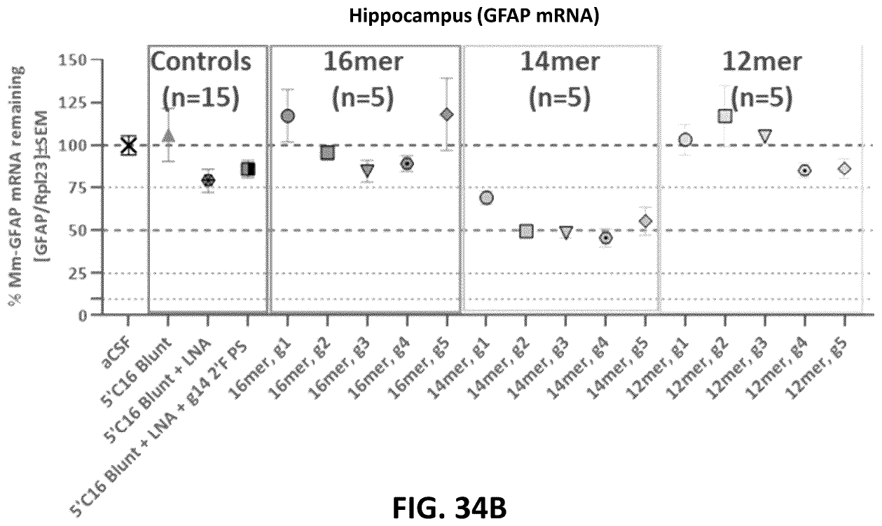

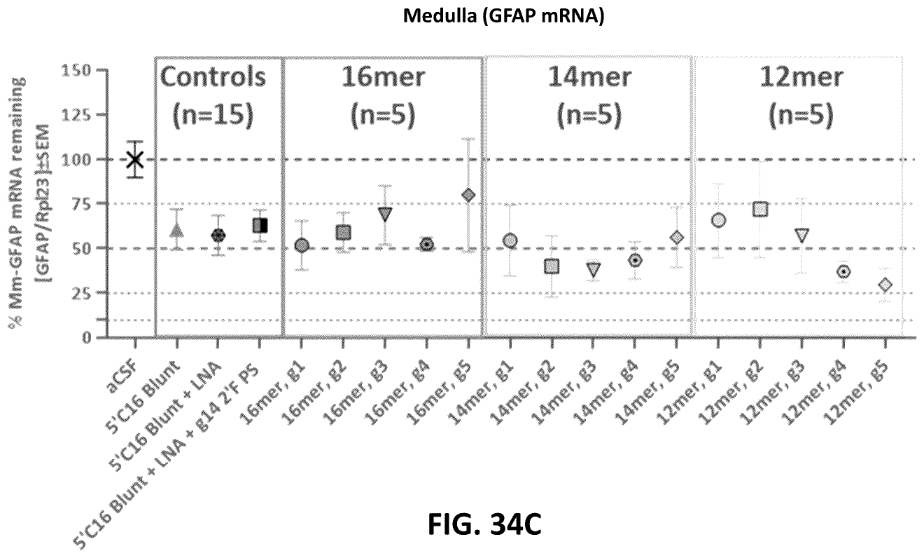

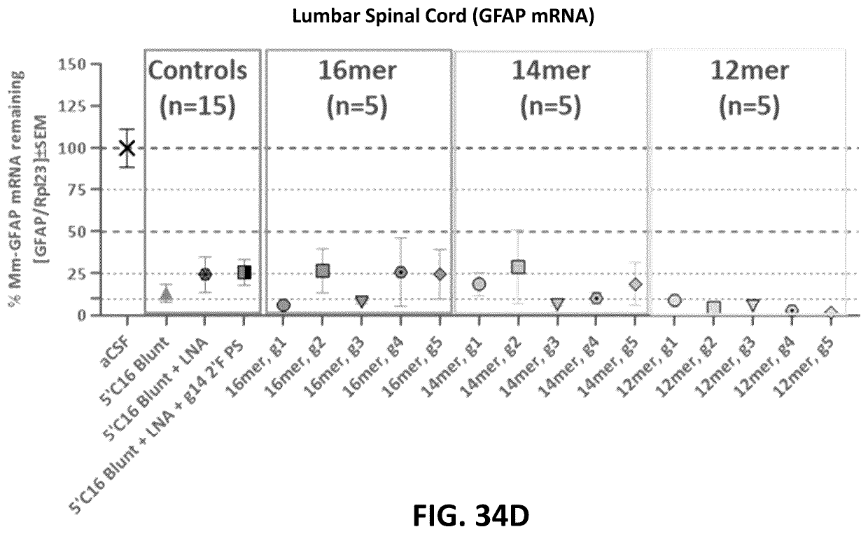

Percent GFAP mRNA expression in the frontal cortex, hippocampus, medulla, and lumbar spinal cord as measured for each treatment group is respectively shown in FIGS. 34A-34D. Silencing of GFAP mRNA expression was observed in all tissues evaluated for most treatment groups. Silencing of GFAP mRNA expression was observed in the lumbar spinal cord and medulla following treatment with all RNAi oligonucleotide-lipid conjugates tested. These results demonstrate the ability of RNAi oligonucleotide-lipid conjugates having truncated sense strands to inhibit astrocyte gene targets in various regions and tissues of the CNS.

### Example 21: Truncated Sense Strand Oligonucleotides with 5′ and 3′ Overhangs for Inhibition of a Neuronal Target Gene in the CNS

RNAi oligonucleotide-lipid conjugates were evaluated to determine the effect of the length of 5′ and/or 3′ truncations on the sense strand of an oligonucleotide. Each oligonucleotide-lipid conjugate tested comprised an antisense strand having a region of complementarity to mRNA encoding TUBB3.

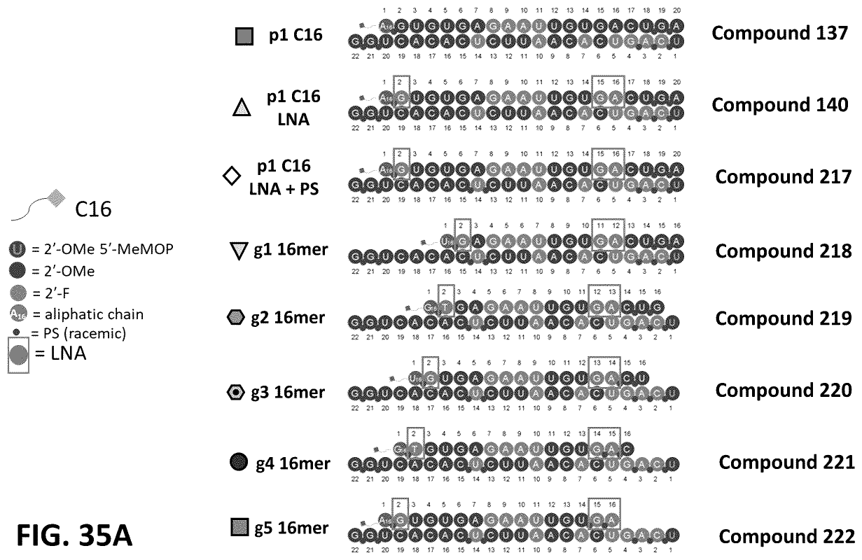

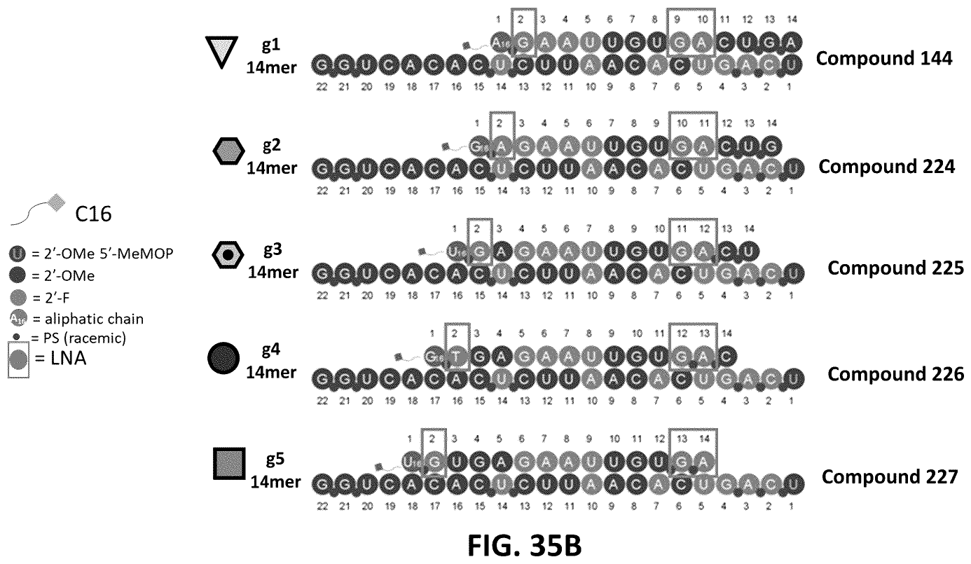

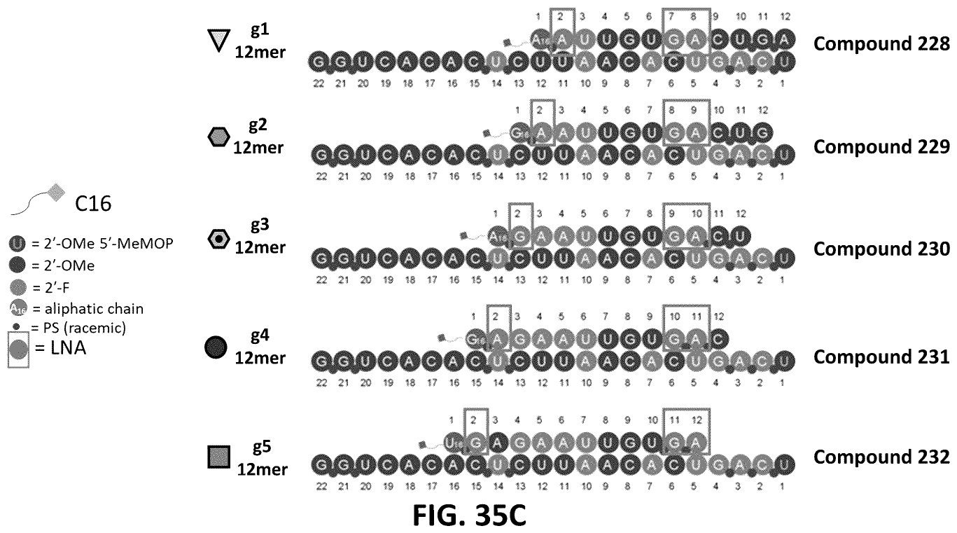

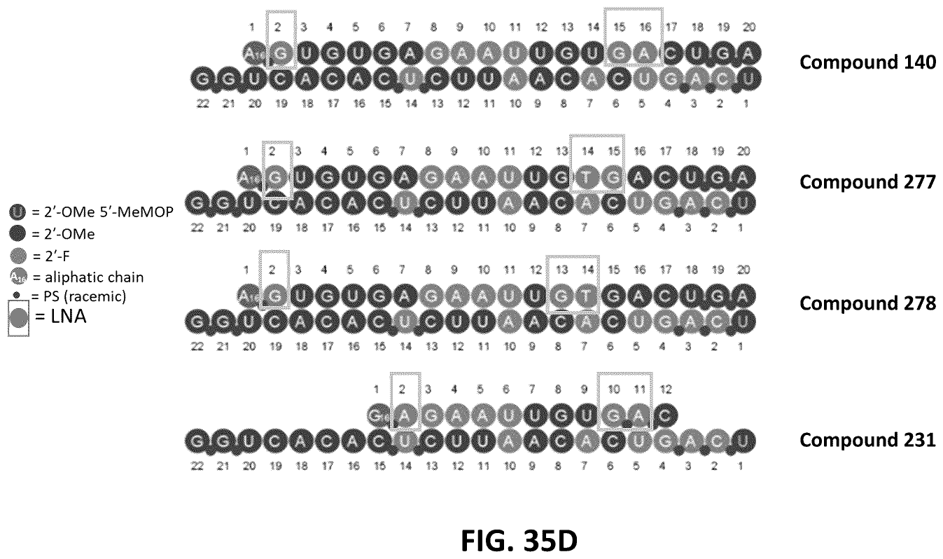

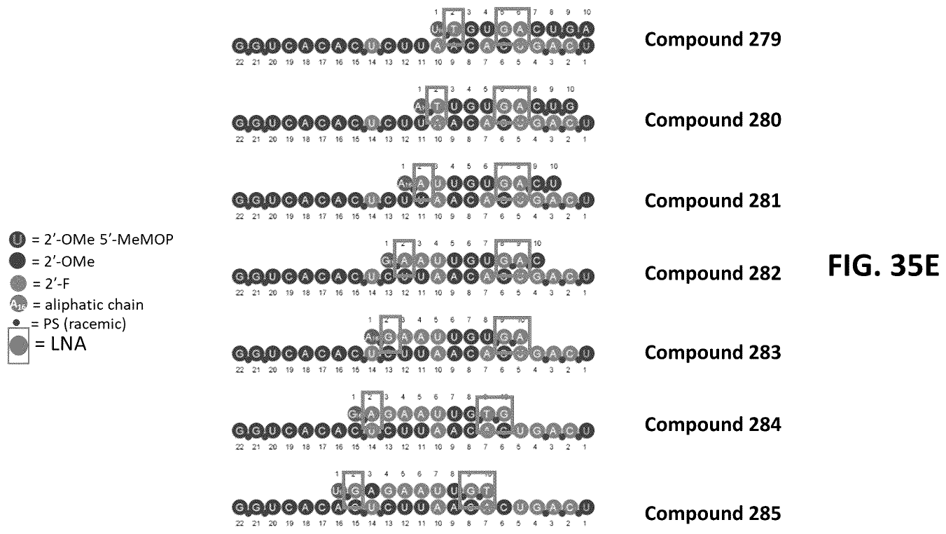

Oligonucleotide-lipid conjugates that were evaluated included those having the structures of Compounds 144, 218-222, 224-232, and 279-285 as shown in FIGS. 35A-35E, compared to the parent oligonucleotide-lipid conjugates of Compounds 137, 140, 217, and 277-278. Each of Compounds 137, 140, 217, and 277-278 contain a blunt end at the 3′ terminus of the sense strand, and a 2-nt antisense strand overhang at the 3′ terminus. Compounds 218-222 and 277-278 comprise a sense strand 16 nucleotides in length. Compounds 140, and 224-227 comprise a sense strand 14 nucleotides in length. Compounds 228-232 comprise a sense strand 12 nucleotides in length. Compounds 279-285 comprise a sense strand 10 nucleotides in length.

Compounds 140, 144, 217-222, 224-232 and 279-283 comprise locked nucleic acids on the sense strand at the nucleotides complementary to nucleotides 5 and 6 of the antisense strand. Compounds 277 and 284 comprise locked nucleic acids on the sense strand at the nucleotides complementary to nucleotides 6 and 7 of the antisense strand. Compounds 278 and 285 comprise locked nucleic acids on the sense strand at the nucleotides complementary to nucleotides 7 and 8 of the antisense strand. Compounds 140, 144, 217-222, 224-232 and 277-285 comprise a locked nucleic acid on position 2 of the sense strand.

Compounds 137, 140, 217, 277, and 278 comprise phosphorothioate linkages between positions: 1 and 2, 18 and 19, and 19 and 20 of the sense strand; 1 and 2, 2 and 3, 3 and 4, 20 and 21, and 21 and 22, of the antisense strand. Compounds 217 and 277-278 comprises additional phosphorothioate linkages between positions 13 and 14 and 14 and 15 of the antisense strand. Compounds 218-222 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. Compounds 144, and 224-227 comprise phosphorothioate linkages between positions: 1 and 2, 12 and 13, and 13 and 14, of the sense strand; 1 and 2, 2 and 3, 3 and 4, 13 and 14, 14 and 15, 20 and 21, and 21 and 22, of the antisense strand. Compounds 228-232 comprise phosphorothioate linkages between positions: 1 and 2, 10 and 11, and 11 and 12, of the sense strand; 1 and 2, 2 and 3, 3 and 4, 13 and 14, 14 and 15, 20 and 21, and 21 and 22, of the antisense strand. Compounds 279-285 comprise phosphorothioate linkages between positions 1 and 2, 8 and 9, and 9 and 10 of the sense strand; 1 and 2, 2 and 3, 3 and 4, 13 and 14, 14 and 15, 20 and 21, and 21 and 22, of the antisense strand.

Compounds 137, 140, 144, 217-222, 224-232, and 278-285 comprise a C16 lipid conjugated at position 1 of the sense strand.

The compounds comprise different length 5′ and 3′ overhangs of the antisense strand as further described below:
(i) Compounds 137, 140, 217, 277, and 278 comprise a 2-nt antisense strand overhang at the 3′ terminus.
(ii) Compound 218 comprises a 6-nt antisense strand overhang at the 3′ terminus.
(iii) Compound 219 comprises a 5-nt antisense strand overhang at the 3′ terminus and a 1-nt antisense strand overhang at the 5′ terminus.
(iv) Compound 220 comprises a 4-nt antisense strand overhang at the 3′ terminus and a 2-nt antisense strand overhang at the 5′ terminus.
(v) Compound 221 comprises a 3-nt antisense strand overhang at the 3′ terminus and a 3-nt antisense strand overhang at the 5′ terminus.
(vi) Compound 222 comprises a 2-nt antisense strand overhang at the 3′ terminus and a 4-nt antisense strand overhang at the 5′ terminus.
(vii) Compound 144 comprises a 8-nt antisense strand overhang at the 3′ terminus.
(viii) Compound 224 comprises a 7-nt antisense strand overhang at the 3′ terminus and a 1-nt antisense strand overhang at the 5′ terminus.
(ix) Compound 225 comprises a 6-nt antisense strand overhang at the 3′ terminus and a 2-nt antisense strand overhang at the 5′ terminus.
(x) Compound 226 comprises a 5-nt antisense strand overhang at the 3′ terminus and a 3-nt antisense strand overhang at the 5′ terminus.
(xi) Compound 227 comprises a 4-nt antisense strand overhang at the 3′ terminus and a 4-nt antisense strand overhang at the 5′ terminus.
(xii) Compound 228 comprises a 10-nt antisense strand overhang.
(xiii) Compound 229 comprises a 9-nt antisense strand overhang at the 3′ terminus and a 1-nt antisense strand overhang at the 5′ terminus.
(xiv) Compound 230 comprises a 8-nt antisense strand overhang at the 3′ terminus and a 2-nt antisense strand overhang at the 5′ terminus.
(xv) Compound 231 comprises a 7-nt antisense strand overhang at the 3′ terminus and a 3-nt antisense strand overhang at the 5′ terminus.
(xvi) Compound 232 comprises a 6-nt antisense strand overhang at the 3′ terminus and a 4-nt antisense strand overhang at the 5′ terminus.
(xvii) Compound 279 comprises a 12-nt antisense strand overhang at the 3′ terminus.
(xviii) Compound 280 comprises a 11-nt antisense strand overhang at the 3′ terminus and a 1-nt antisense strand overhang at the 5′ terminus.
(xix) Compound 281 comprises a 10-nt antisense strand overhang at the 3′ terminus and a 2-nt antisense strand overhang at the 5′ terminus.
(xx) Compound 282 comprises a 9-nt antisense strand overhang at the 3′ terminus and a 3-nt antisense strand overhang at the 5′ terminus.
(xxi) Compound 283 comprises a 8-nt antisense strand overhang at the 3′ terminus and a 4-nt antisense strand overhang at the 5′ terminus.
(xxii) Compound 284 comprises a 7-nt antisense strand overhang at the 3′ terminus and a 5-nt antisense strand overhang at the 5′ terminus.
(xxiii) Compound 285 comprises a 6-nt antisense strand overhang at the 3′ terminus and a 6-nt antisense strand overhang at the 5′ terminus.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 21. Control mice received artificial cerebrospinal fluid (aCSF) only.

TABLE 21
TUBB3 RNAi oligonucleotides administered to animals

| | | Sense | | Antisense | |
|---|---|---|---|---|---|
| | Compound | SEQ ID NOS | | SEQ ID NOS | |
| Group | ID | Unmod | Mod | Unmod | Mod |

Controls

| | | | | | |
|---|---|---|---|---|---|
| | aCSF | — | — | — | — |
| 5′C16 Blunt | 173 | 583 | 586 | 596 | 597 |
| 5′C16 Blunt + LNA | 176 | 583 | 589 | 596 | 597 |
| 5′C16 Blunt + LNA + | 217 | 372 | 541 | 324 | 533 |
| g14 2′F PS | | | | | |

16mer Sense Strands

| | | | | | |
|---|---|---|---|---|---|
| 16mer, g1 | 218 | 549 | 554 | 324 | 533 |
| 16mer, g2 | 219 | 630 | 686 | 324 | 533 |
| 16mer, g3 | 220 | 63 | 687 | 324 | 533 |
| 16mer, g4 | 221 | 632 | 688 | 324 | 533 |
| 16mer, g5 | 222 | 443 | 465 | 324 | 533 |

14mer Sense Strands

| | | | | | |
|---|---|---|---|---|---|
| 14mer, g1 | 144 | 537 | 545 | 324 | 533 |
| 14mer, g2 | 224 | 633 | 689 | 324 | 533 |
| 14mer, g3 | 225 | 634 | 690 | 324 | 533 |
| 14mer, g4 | 226 | 635 | 691 | 324 | 533 |
| 14mer, g5 | 227 | 636 | 692 | 324 | 533 |

12mer Sense Strands

| | | | | | |
|---|---|---|---|---|---|
| 12mer, g1 | 228 | 557 | 562 | 324 | 533 |
| 12mer, g2 | 229 | 637 | 693 | 324 | 533 |
| 12mer, g3 | 230 | 638 | 694 | 324 | 533 |
| 12mer, g4 | 231 | 639 | 695 | 324 | 533 |
| 12mer, g5 | 232 | 640 | 696 | 324 | 533 |

10mer Sense Strands and controls

| | | | | | |
|---|---|---|---|---|---|
| 5′ C16 Full, LNA P2, | 277 | 644 | 729 | 324 | 533 |
| P14, P15 (g14 2′F | | | | | |
| PS) | | | | | |
| 5′ C16 Full, LNA P2, | 278 | 644 | 730 | 324 | 533 |
| P13, P14 (g14 2′F | | | | | |
| PS) | | | | | |
| 10mer, g1 | 279 | 665 | 731 | 324 | 533 |
| 10mer, g2 | 280 | 666 | 732 | 324 | 533 |
| 10mer, g3 | 281 | 667 | 733 | 324 | 533 |
| 10mer, g4 | 282 | 668 | 734 | 324 | 533 |
| 10mer, g5 | 283 | 669 | 735 | 324 | 533 |
| 10mer, g6 | 284 | 670 | 736 | 324 | 533 |
| 10mer, g7 | 285 | 671 | 737 | 324 | 533 |

Target mRNA knockdown was assessed 28 days post-injection. RNA was extracted from CNS tissue and murine TUBB3 mRNA levels were determined as described in Example 2.

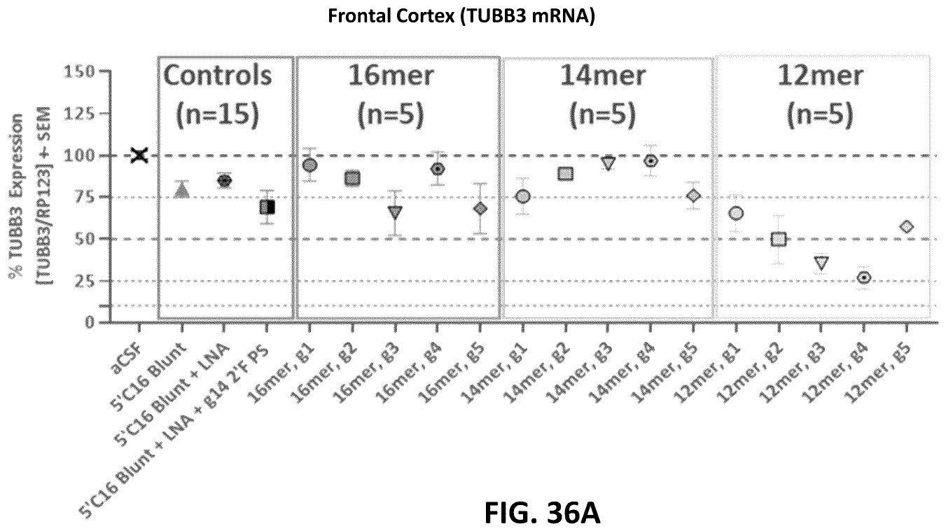

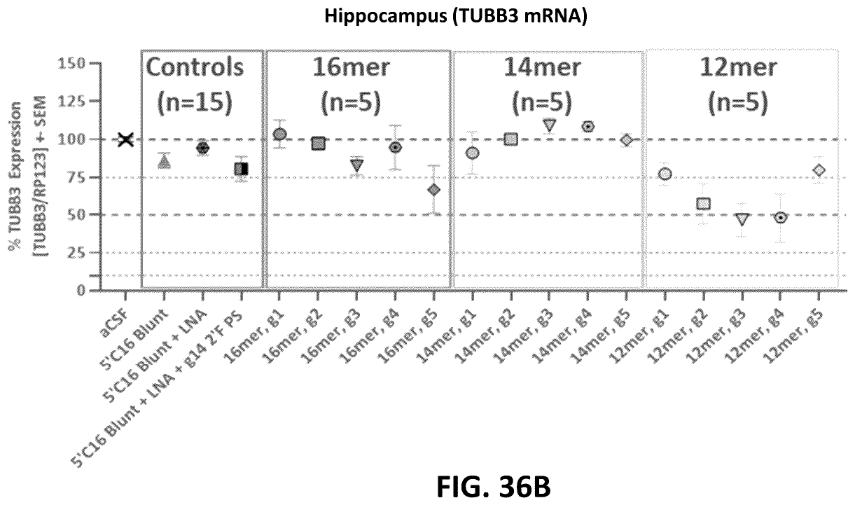

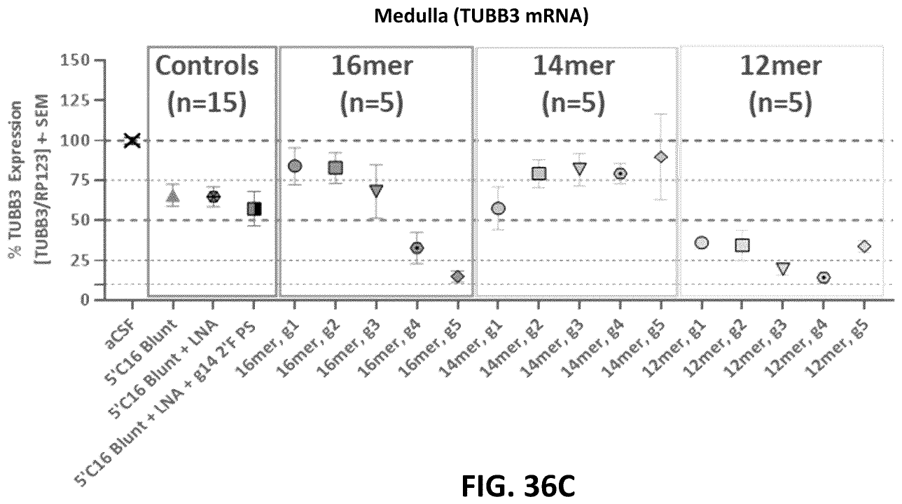

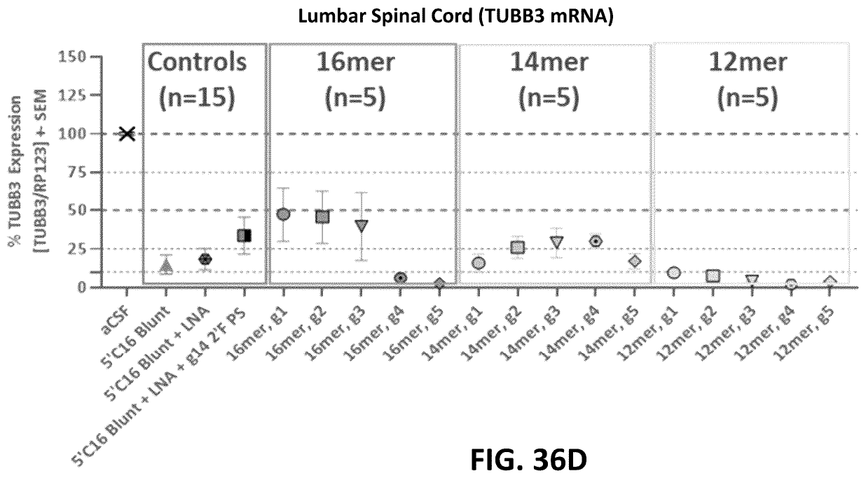

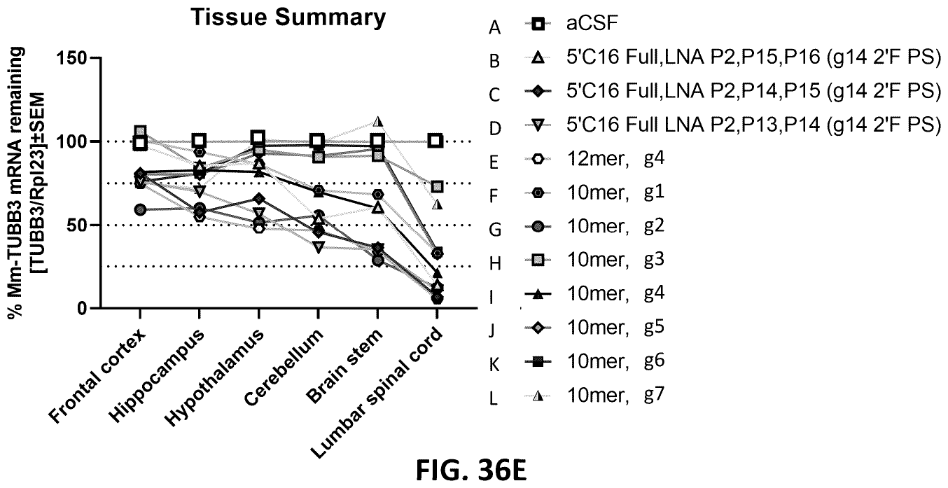

Percent TUBB3 mRNA expression remaining in the frontal cortex, hippocampus, medulla, and lumbar spinal cord following treatment with Compounds 173, 176, and 217-232 is respectively shown in FIGS. 36A-36D. Percent TUBB3 mRNA expression remaining in the frontal cortex, hippocampus, hypothalamus, cerebellum, brain stem, and lumbar spinal cord following treatment with Compounds 140, 277, 278, 231, and 279-285 is respectively shown in FIG. 36E. Silencing of TUBB3 mRNA expression was observed in all tissues evaluated for most treatment groups. Silencing of TUBB3 mRNA expression was observed in the lumbar spinal cord following treatment with all RNAi oligonucleotides-lipid conjugates tested. Inhibition was further observed in the medulla, hippocampus, and frontal cortex following treatment with all RNAi oligonucleotide-lipid conjugates tested comprising a sense strand 12 nucleotides in length. Silencing of TUBB3 mRNA expression was observed in all CNS regions evaluated following treatment with Compounds 228-232, Compound 281, or Compound 283. Together, these results demonstrate the ability of RNAi oligonucleotide-lipid conjugates having truncated sense strands to inhibit neuronal gene targets in various regions and tissues of the CNS.

### Example 22: Impact of Lipid Position on Truncated Sense Strand Oligonucleotides with 5′ and 3′ Overhangs for Inhibition of an Astrocyte Target Gene in the CNS

RNAi oligonucleotide-lipid conjugates were evaluated to determine the effect of lipid position on oligonucleotides with 5′ and/or 3′ truncations on the sense strand. Each oligonucleotide-lipid conjugate tested comprised an antisense strand having a region of complementarity to mRNA encoding GFAP.

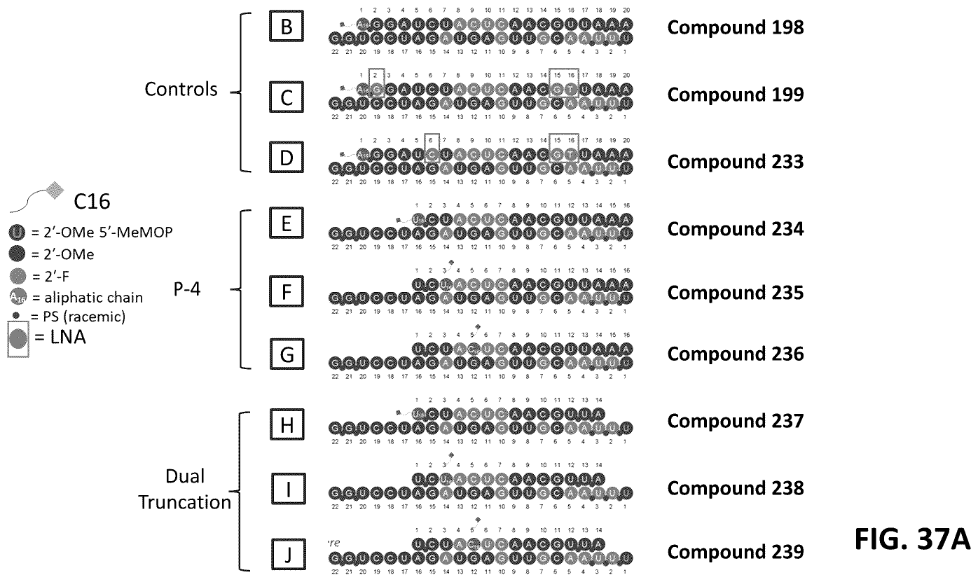

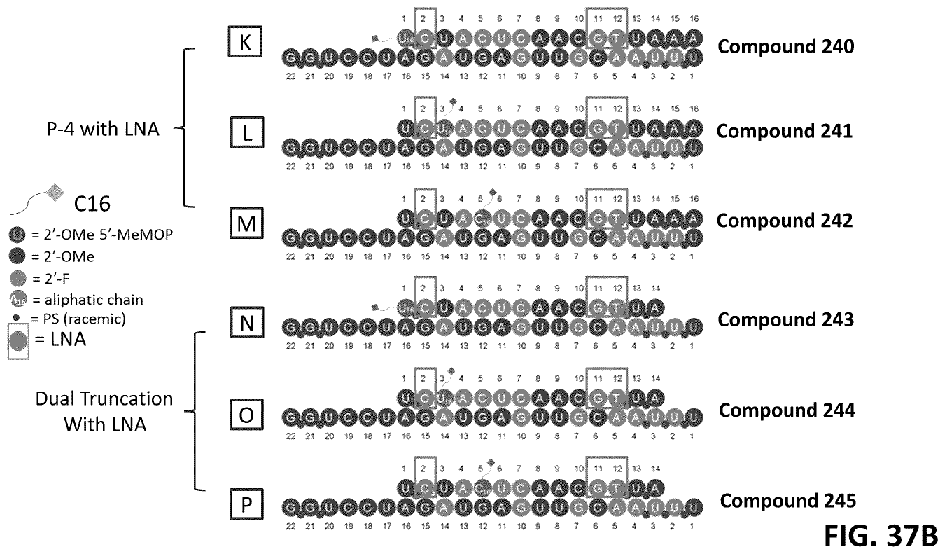

Oligonucleotide-lipid conjugates that were evaluated included those having the structures of Compounds 234-245 as shown in FIGS. 37A-37B, compared to the parent oligonucleotide-lipid conjugates of Compounds 173, 176, and 233. Each of Compounds 173, 176, 233-236, and 240-242 contain a blunt end at the 3′ terminus of the sense strand. Compounds 173, 176, and 233 contain a 2-nt overhang on the 3′ terminus of the antisense strand. Compounds 234-245 contain a 6-nt overhang on the 3′ terminus of the antisense strand. Compounds 234-236 and 240-242 comprise a sense strand 16 nucleotides in length. Compounds and 237-239 and 243-245 comprise a sense strand 14 nucleotides in length.

Compound 176 comprises locked nucleic acids on the sense strand at nucleotide positions 2, 15, and 16. Compound 233 comprises locked nucleic acids on the sense strand at nucleotide positions 6, 15, and 16. Compounds 240-245 comprise locked nucleic acids on the sense strand at the nucleotide positions 2, 11, and 12.

Compounds 173, 176, and 233 comprise phosphorothioate linkages between positions: 1 and 2, 18 and 19, and 19 and 20 of the sense strand; 1 and 2, 2 and 3, 3 and 4, 20 and 21, and 21 and 22, of the antisense strand. Compound 234-236 and 240-242 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. Compounds 237-239 and 243-245 comprise phosphorothioate linkages between positions: 1 and 2, 12 and 13, and 13 and 14, of the sense strand; 1 and 2, 2 and 3, 3 and 4, 13 and 14, 14 and 15, 20 and 21, and 21 and 22, of the antisense strand.

The compounds comprise a C16 lipid conjugated at a different position of the sense strand as further described below:
(i) Compounds 173, 176, 233, 234, 237, 240, and 243 comprise a C16 lipid at position 1.
(ii) Compounds 235, 238, 241, and 244 comprise a C16 lipid at position 3.
(iii) Compounds 236, 239, 242, and 245 comprise a C16 lipid at position 5.

To evaluate the RNAi oligonucleotide-lipid conjugates, 6 to 10 week old C57BL6 mice were given a single intrathecal (i.t.) injection of 100 μg oligonucleotide-lipid conjugate formulated in artificial cerebrospinal fluid (aCSF). The RNAi oligonucleotide-lipid conjugate administered to each animal group is outlined in Table 22. Control mice received artificial cerebrospinal fluid (aCSF) only.

TABLE 22
GFAP RNAi oligonucleotides administered to animals

| | Compound | Sense SEQ ID NOS | | Antisense SEQ ID NOS | |
|---|---|---|---|---|---|
| Group | ID | Unmod | Mod | Unmod | Mod |

Controls

| | | | | | |
|---|---|---|---|---|---|
| | aCSF | — | — | — | — |
| B | 173 | 583 | 586 | 596 | 597 |
| C | 176 | 583 | 589 | 596 | 597 |
| D | 233 | 614 | 697 | 596 | |

P-4

| | | | | | |
|---|---|---|---|---|---|
| E | 234 | 641 | 698 | 596 | 597 |
| F | 235 | 641 | 699 | 596 | 597 |
| G | 236 | 641 | 700 | 596 | 597 |

Dual Truncation

| | | | | | |
|---|---|---|---|---|---|
| H | 237 | 642 | 701 | 596 | 597 |
| I | 238 | 642 | 702 | 596 | 597 |
| J | 239 | 642 | 703 | 596 | 597 |

P-4 with LNA

| | | | | | |
|---|---|---|---|---|---|
| K | 240 | 615 | 672 | 596 | 597 |
| L | 241 | 615 | 704 | 596 | 597 |
| M | 242 | 615 | 705 | 596 | 597 |

Dual Truncation with LNA

| | | | | | |
|---|---|---|---|---|---|
| N | 243 | 622 | 678 | 596 | 597 |
| O | 244 | 622 | 706 | 596 | 597 |
| P | 245 | 622 | 707 | 596 | 597 |

Target knockdown was assessed 28 days post-injection. RNA was extracted from CNS tissue and murine GFAP mRNA levels were determined as described in Example 17.

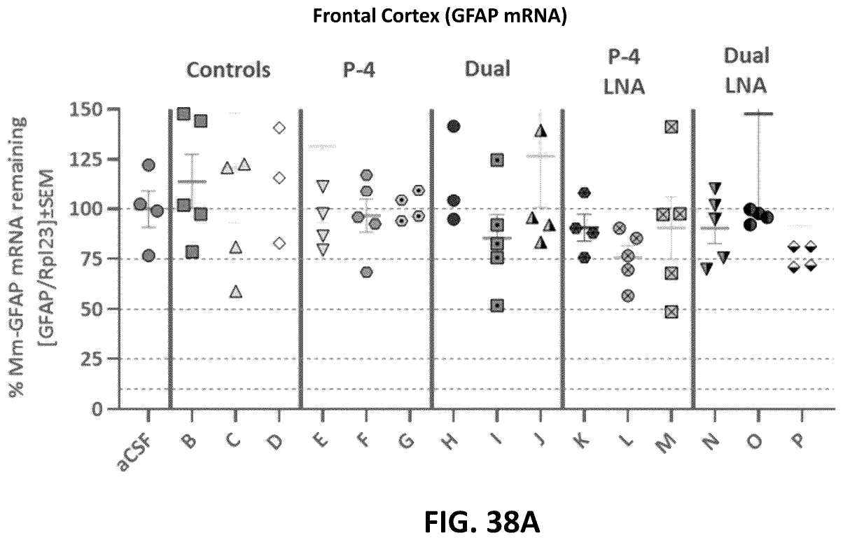

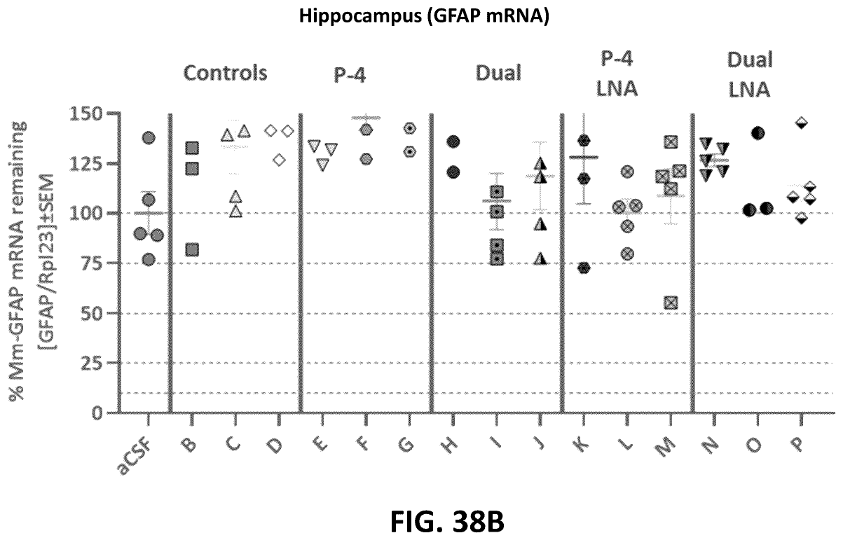

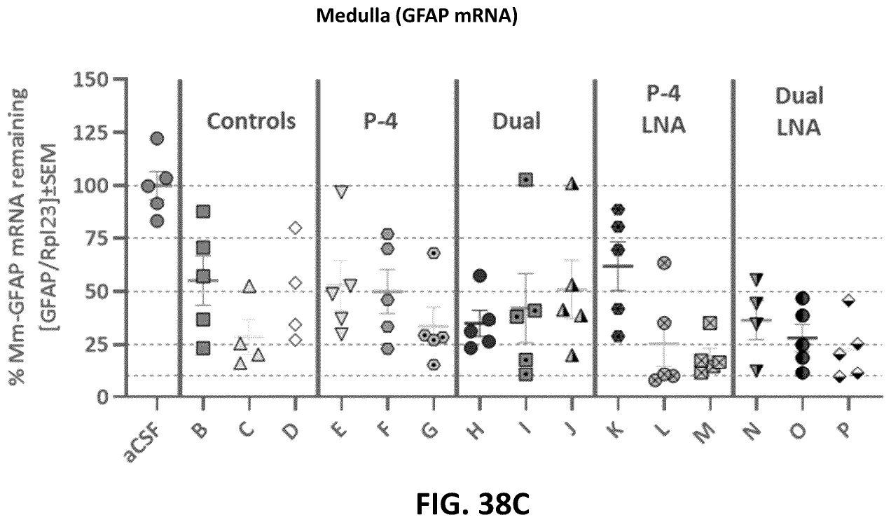

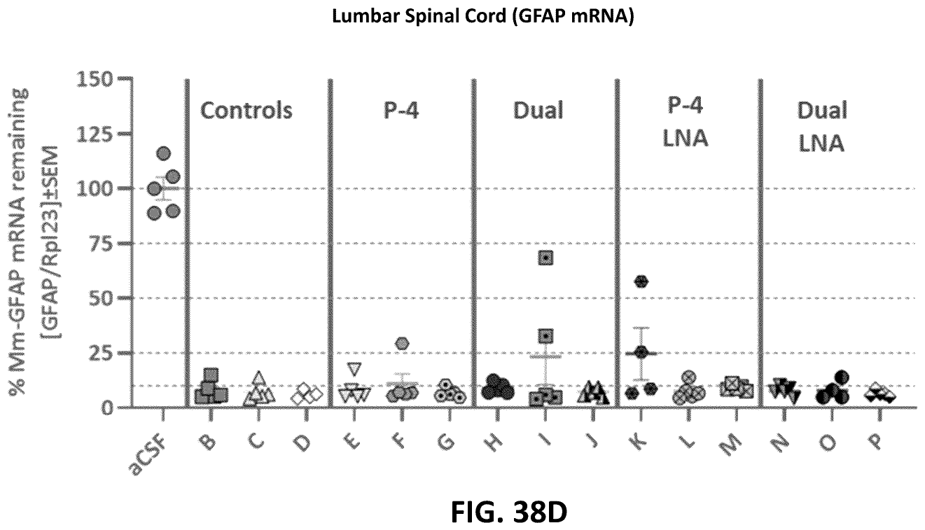

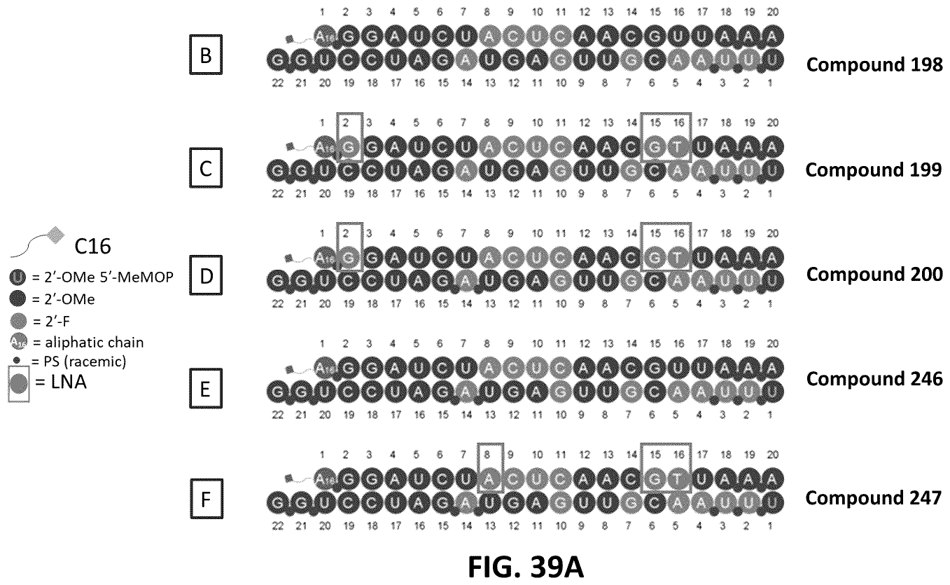

Percent GFAP mRNA expression in the frontal cortex, hippocampus, medulla, and lumbar spinal cord as measured for each treatment group is respectively shown in FIGS. 38A-38D. Silencing of GFAP mRNA expression was observed in medulla and lumbar spinal cord following treatment with all RNAi oligonucleotide-lipid conjugates tested. Inclusion of locked nucleic acids in oligonucleotides comprising 5′ and 3′ truncations improved the knockdown of the astrocyte target.

### Example 23: Impact of Lipid Position on Truncated Sense Strands with Locked Nucleic Acids for Inhibition of an Astrocyte Target Gene in the CNS

RNAi oligonucleotide-lipid conjugates were evaluated to determine the effect of lipid position on oligonucleotides locked nucleic acids and 5′ and/or 3′ truncations on the sense strand. Each oligonucleotide-lipid conjugate tested comprised an antisense strand having a region of complementarity to mRNA encoding GFAP.

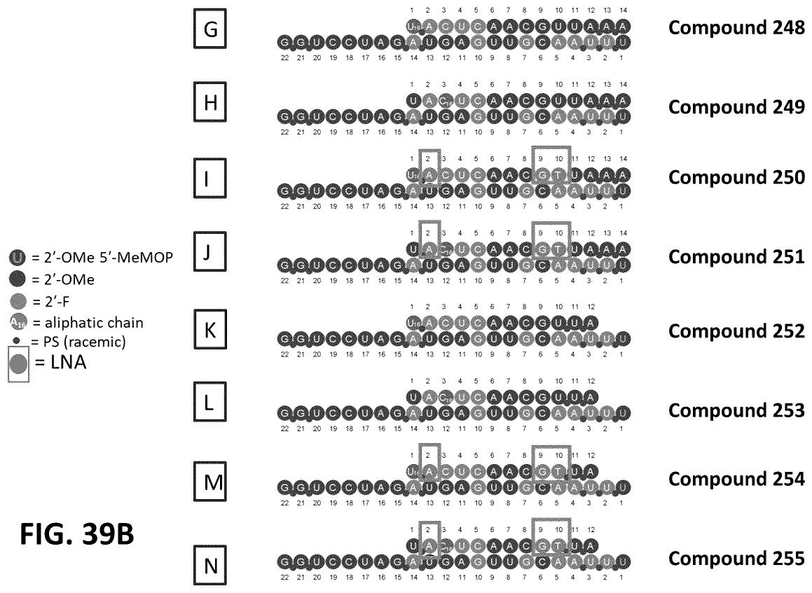

Oligonucleotide-lipid conjugates that were evaluated included those having the structures of Compounds 248-255 as shown in FIGS. 39A-39B, compared to the parent oligonucleotide-lipid conjugates of Compounds 173, 176, and 200 and 246-247. Each of Compounds 173, 176, and 200 and 246-251 contain a blunt end at the 3′ terminus of the sense strand. Compounds 252-255 contain a 2-nt overhang on the 5′ terminus of the antisense strand. Compounds 248-255 contain a 8-nt overhang on the 3′ terminus of the antisense strand. Compounds 173, 176, 200 and 246-247 comprise a sense strand 14 nucleotides in length. Compounds 248-250 comprise a sense strand 14 nucleotides in length. Compounds and 253-255 comprise a sense strand 12 nucleotides in length.

Compounds 176, 200 comprise locked nucleic acids on the sense strand at nucleotide positions 2, 15, and 16. Compound 247 comprises locked nucleic acids on the sense strand at nucleotide positions 8, 15, and 16. Compounds 250, 251, 254, and 255 comprise locked nucleic acids on the sense strand at the nucleotide positions 2, 9, and 10.

Compounds 173, 176, 200, 246, and 233 comprise phosphorothioate linkages between positions: 1 and 2, 18 and 19, and 19 and 20 of the sense strand. Compound 248, 250-252, and 254-255 comprise phosphorothioate linkages between positions: 1 and 2, 12 and 13, and 13 and 14, of the sense strand. Compounds 249 and 253 comprise phosphorothioate linkages between positions 12 and 13 and 13 and 14. Compounds 173, 176, 200, and 246-255 comprise phosphorothioate linkages between positions 1 and 2, 2 and 3, 3 and 4, 13 and 14, 14 and 15, 20 and 21, and 21 and 22, of the antisense strand.

The compounds comprise a C16 lipid conjugated at a different position of the sense strand as further described below:
(i) Compounds 173, 176, 200, 246-248, 250, 252, and 254 comprise a C16 lipid at position 1.
(ii) Compounds 249, 251, 253, and 255, comprise a C16 lipid at position 3.

To evaluate the RNAi oligonucleotide-lipid conjugates, 6 to 10 week old C57BL6 mice were given a single intrathecal (i.t.) injection of 100 μg oligonucleotide-lipid conjugate formulated in artificial cerebrospinal fluid (aCSF). The RNAi oligonucleotide-lipid conjugate administered to each animal group is outlined in Table 23. Control mice received artificial cerebrospinal fluid (aCSF) only.

TABLE 23
GFAP RNAi oligonucleotides administered to animals

| | Sense | | Antisense | |
|---|---|---|---|---|
| Compound | SEQ ID NOS | | SEQ ID NOS | |
| ID | Unmod | Mod | Unmod | Mod |

Controls

| | | | | | |
|---|---|---|---|---|---|
| | aCSF | — | — | — | — |
| B | 173 | 583 | 586 | 596 | 597 |
| C | 176 | 583 | 589 | 596 | 597 |
| D | 200 | 614 | 589 | 596 | 598 |
| E | 246 | 583 | 586 | 596 | 598 |
| F | 247 | 614 | 708 | 596 | 598 |

P-6

| | | | | | |
|---|---|---|---|---|---|
| G | 248 | 584 | 709 | 596 | 598 |
| H | 249 | 584 | 710 | 596 | 598 |

P-6 LNA

| | | | | | |
|---|---|---|---|---|---|
| I | 250 | 620 | 593 | 596 | 598 |
| J | 251 | 620 | 711 | 596 | 598 |

Dual Truncation

| | | | | | |
|---|---|---|---|---|---|
| K | 252 | 643 | 712 | 596 | 598 |
| L | 253 | 643 | 713 | 596 | 598 |

Dual Truncation with LNA

| | | | | | |
|---|---|---|---|---|---|
| M | 254 | 627 | 683 | 596 | 598 |
| N | 255 | 627 | 714 | 596 | 598 |

Target knockdown was assessed 28 days post-injection. RNA was extracted from CNS tissue and murine GFAP mRNA levels were determined as described in Example 17.

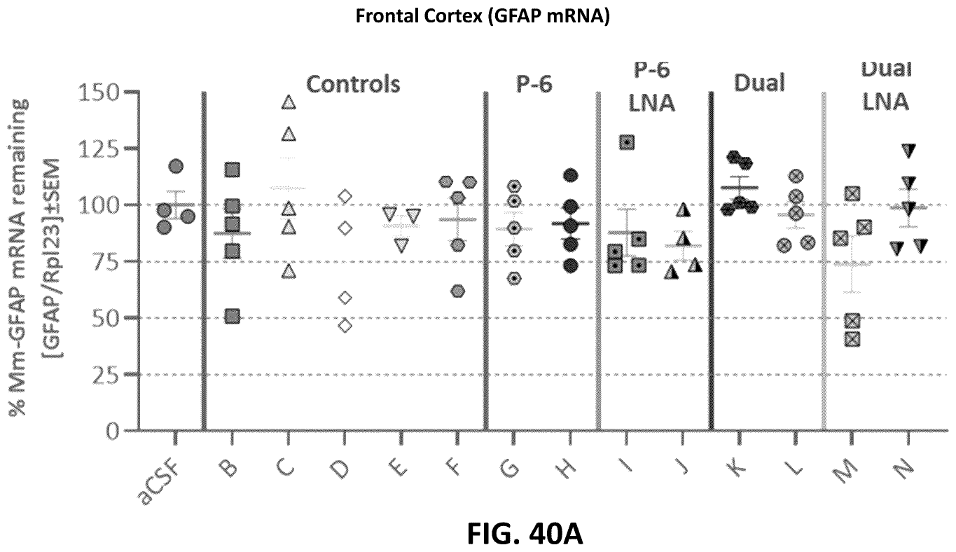

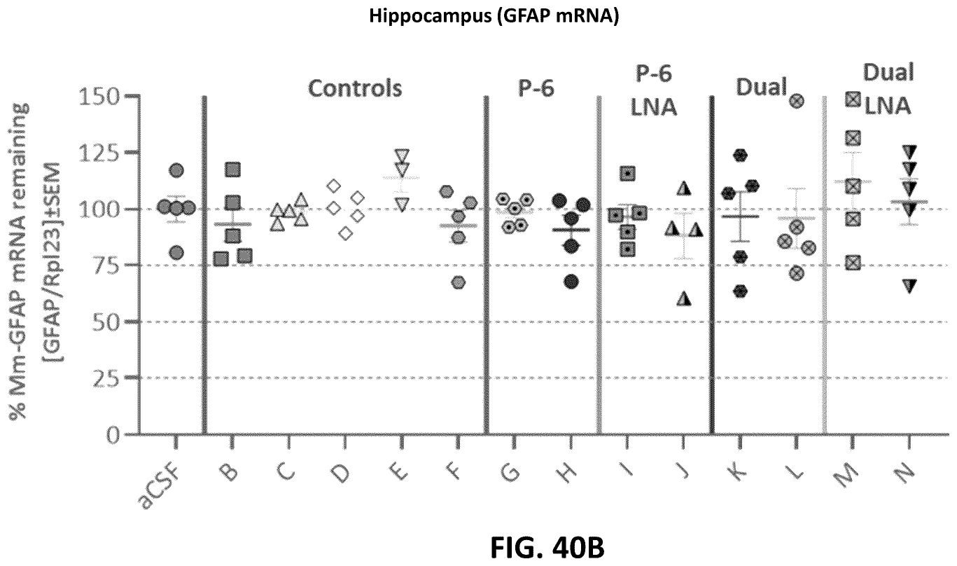

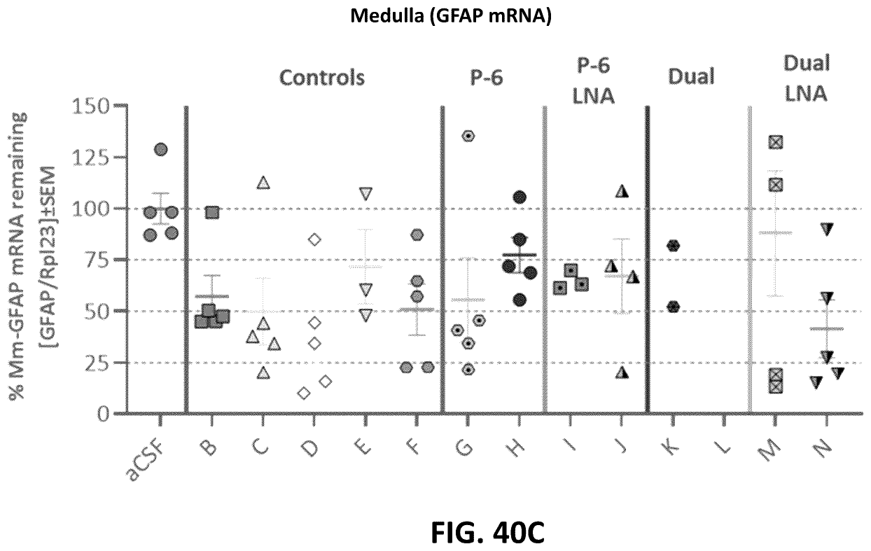

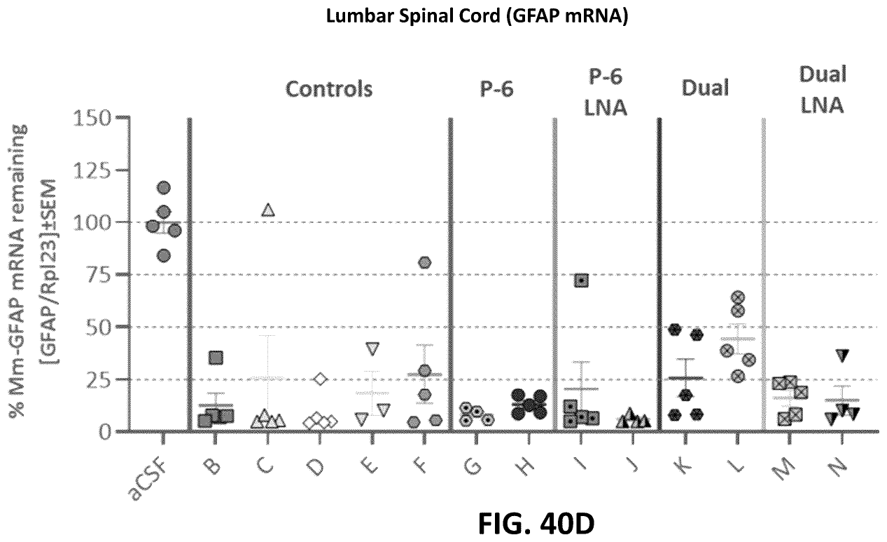

Percent GFAP mRNA expression in the frontal cortex, hippocampus, medulla, and lumbar spinal cord as measured for each treatment group is respectively shown in FIGS. 40A-40D. Silencing of GFAP mRNA expression was observed in medulla and lumbar spinal cord following treatment with all RNAi oligonucleotide-lipid conjugates tested. These results demonstrate the ability of RNAi oligonucleotide-lipid conjugates having truncated sense strand to inhibit astrocyte gene targets in various regions and tissues of the CNS.

### Example 24: Impact of Lipid Position on Truncated Sense Strand Oligonucleotides with 5′ and 3′ Overhangs for Inhibition of a Neuronal Target Gene in the CNS

RNAi oligonucleotide-lipid conjugates were evaluated to determine the effect of lipid position on oligonucleotides with 5′ and/or 3′ truncations on the sense strand. Each oligonucleotide-lipid conjugate tested comprised an antisense strand having a region of complementarity to mRNA encoding TUBB3.

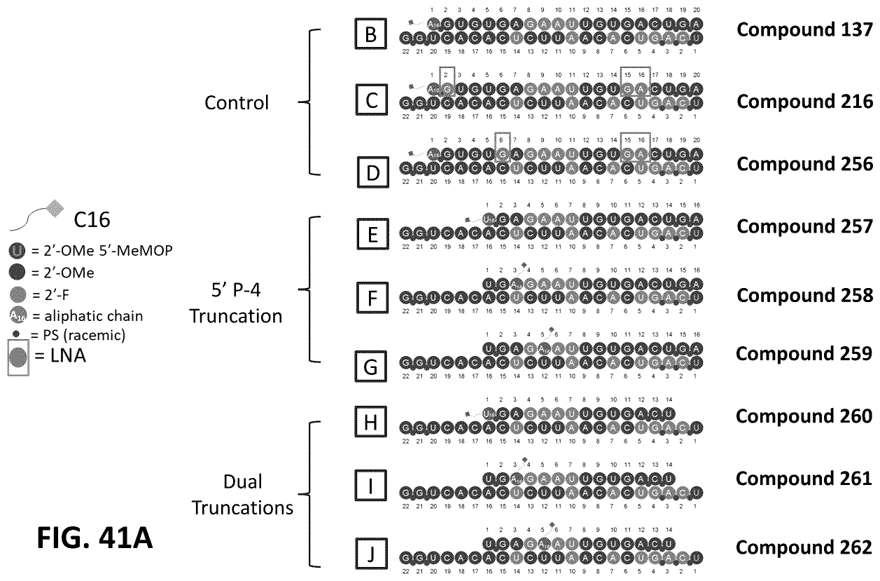

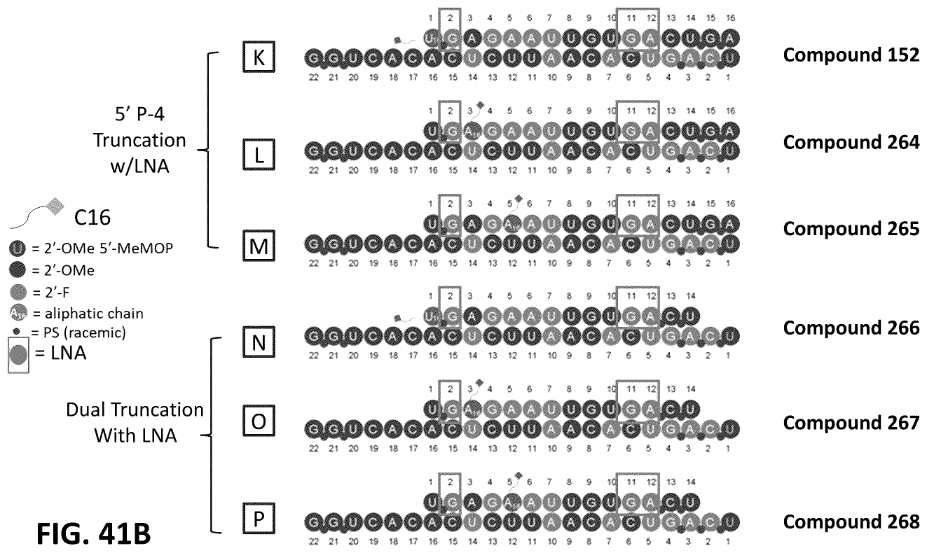

Oligonucleotide-lipid conjugates that were evaluated included those having the structures of Compounds 257-268 as shown in FIGS. 41A-41B, compared to the parent oligonucleotide-lipid conjugates of Compounds 137, 140, and 256. Each of Compounds 137, 140, 152, 256-259, and 264-265 contain a blunt end at the 3′ terminus of the sense strand. Compounds 137, 140, and 256 contain a 2-nt overhang on the 3′ terminus of the antisense strand. Compounds 257-268 contain a 6-nt overhang on the 3′ terminus of the antisense strand. Compounds 152, 257-259 and 264-265 comprise a sense strand 16 nucleotides in length. Compounds and 260-262 and 266-268 comprise a sense strand 14 nucleotides in length.

Compound 140 comprises locked nucleic acids on the sense strand at nucleotide positions 2, 15, and 16. Compound 256 comprises locked nucleic acids on the sense strand at nucleotide positions 6, 15, and 16. Compounds 152, and 264-268 comprise locked nucleic acids on the sense strand at the nucleotide positions 2, 11, and 12.

Compounds 137, 140, and 256 comprise phosphorothioate linkages between positions: 1 and 2, 18 and 19, and 19 and 20 of the sense strand; 1 and 2, 2 and 3, 3 and 4, 20 and 21, and 21 and 22, of the antisense strand. Compounds 152, 257-259, and 264-265 comprise phosphorothioate linkages between positions: 1 and 2, 14 and 15, and 15 and 16, of the sense strand; 1 and 2, 2 and 3, 3 and 4, 20 and 21, and 21 and 22, of the antisense strand. Compounds 260-262 and 266-268 comprise phosphorothioate linkages between positions: 1 and 2, 12 and 13, and 13 and 14, of the sense strand; 1 and 2, 2 and 3, 3 and 4, 13 and 14, 14 and 15, 20 and 21, and 21 and 22, of the antisense strand.

The compounds comprise a C16 lipid conjugated at a different position of the sense strand as further described below:
(i) Compounds 137, 140, 256, 257, 260, 152, and 266 comprise a C16 lipid at position 1.
(ii) Compounds 258, 261, 264, and 267 comprise a C16 lipid at position 3.
(iii) Compounds 259, 262, 265, and 268 comprise a C16 lipid at position 5.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 24. Control mice received artificial cerebrospinal fluid (aCSF) only.

TABLE 24
TUBB 3 RNAi oligonucleotides administered to animals

| | Sense | | Antisense | |
|---|---|---|---|---|
| Compound | SEQ ID NOS | | SEQ ID NOS | |
| ID | Unmod | Mod | Unmod | Mod |

Controls

| | | | | | |
|---|---|---|---|---|---|
| | aCSF | — | — | — | — |
| B | 137 | 372 | 386 | 324 | 352 |
| C | 140 | 372 | 541 | 324 | 352 |
| D | 256 | 372 | 715 | 324 | 352 |

5′ P-4 Truncation

| | | | | | |
|---|---|---|---|---|---|
| E | 257 | 549 | 551 | 324 | 352 |
| F | 258 | 549 | 608 | 324 | 352 |
| G | 259 | 549 | 610 | 324 | 352 |

Dual Truncations

| | | | | | |
|---|---|---|---|---|---|
| H | 260 | 634 | 716 | 324 | 352 |
| I | 261 | 634 | 717 | 324 | 352 |
| J | 262 | 634 | 718 | 324 | 352 |

5′ P-4 Truncation with LNA

| | | | | | |
|---|---|---|---|---|---|
| K | 152 | 549 | 554 | 324 | 352 |
| L | 264 | 549 | 718 | 324 | 352 |
| M | 265 | 549 | 720 | 324 | 352 |

Dual Truncation with LNA

| | | | | | |
|---|---|---|---|---|---|
| N | 266 | 634 | 690 | 324 | 352 |
| O | 267 | 634 | 721 | 324 | 352 |
| P | 268 | 634 | 722 | 324 | 352 |

Target knockdown was assessed 28 days post-injection. RNA was extracted from CNS tissue and murine TUBB3 mRNA levels were determined as described in Example 2.

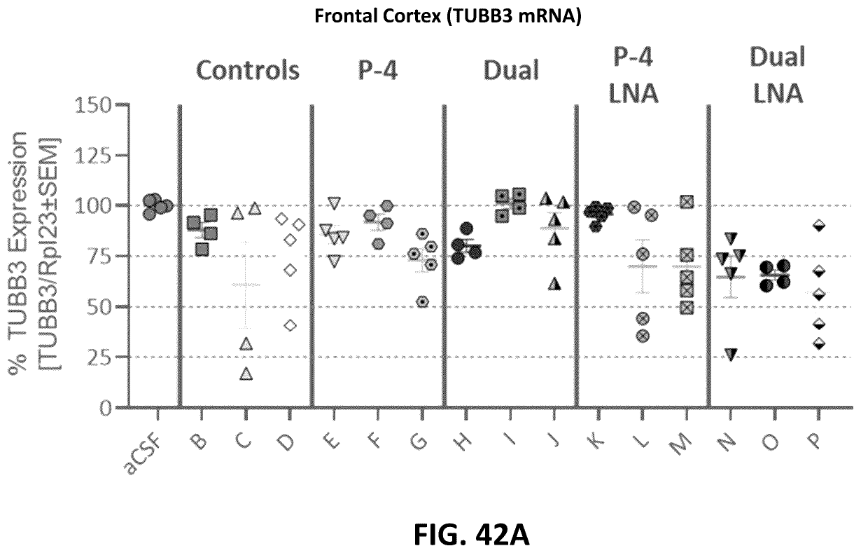

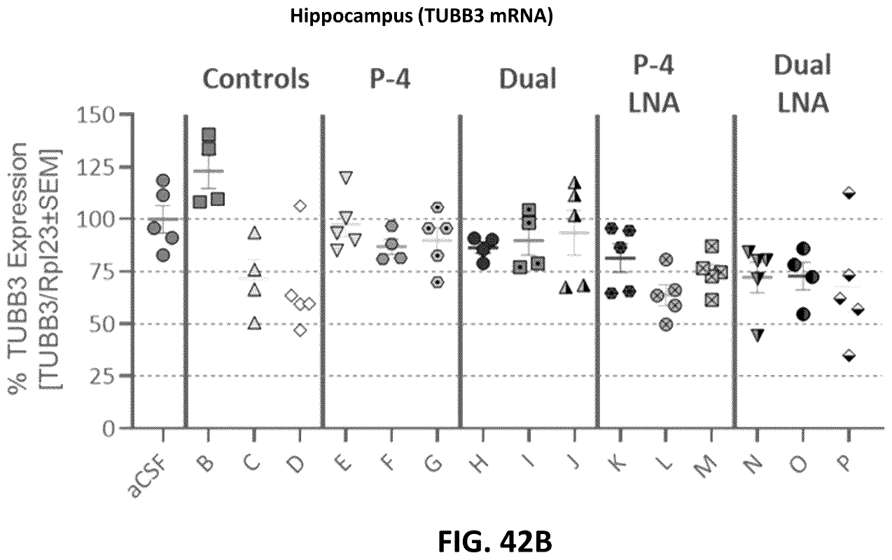

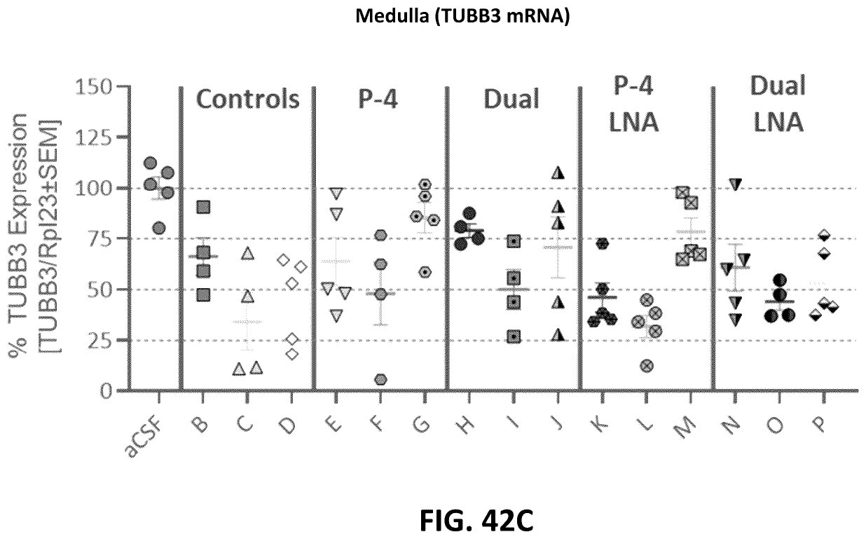

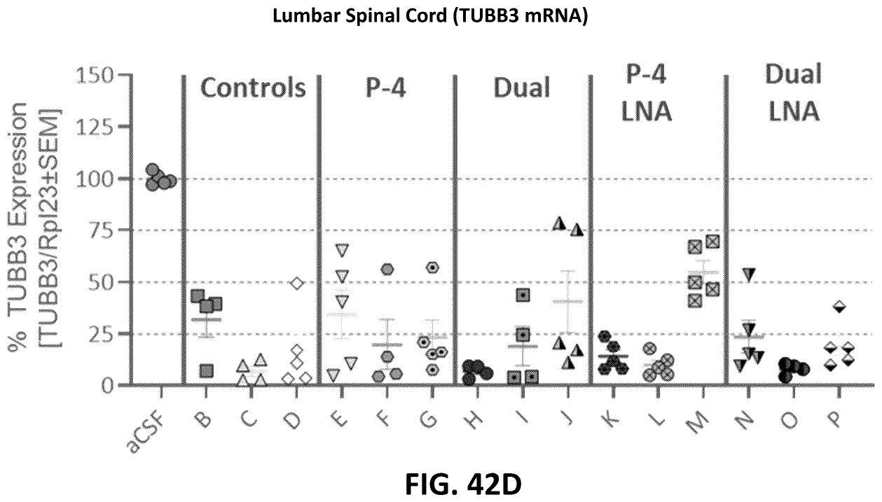

Percent TUBB3 mRNA expression in the frontal cortex, hippocampus, medulla, and lumbar spinal cord as measured for each treatment group is respectively shown in FIGS. 42A-42D. Silencing of TUBB3 mRNA expression was observed in the lumbar spinal cord and medulla following treatment with all RNAi oligonucleotide-lipid conjugates tested. Inhibition was further observed in hippocampus and frontal cortex following treatment with all RNAi oligonucleotide-lipid conjugates tested comprising a sense strand having a 5′ and 3′ truncation and LNAs (e.g., Compounds 266-268).

### Example 25: Impact of Lipid Position on Truncated Sense Strands with Locked Nucleic Acids for Inhibition of a Neuronal Target Gene in the CNS

RNAi oligonucleotide-lipid conjugates were evaluated to determine the effect of lipid position on oligonucleotides with and without locked nucleic acids and 5′ and/or 3′ truncations on the sense strand. Each oligonucleotide-lipid conjugate tested comprised an antisense strand having a region of complementarity to mRNA encoding TUBB3.

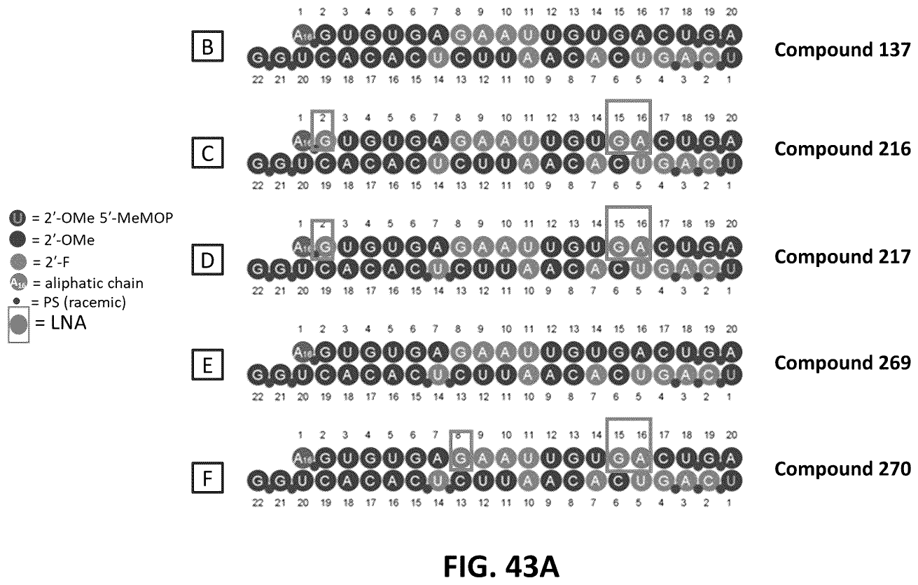

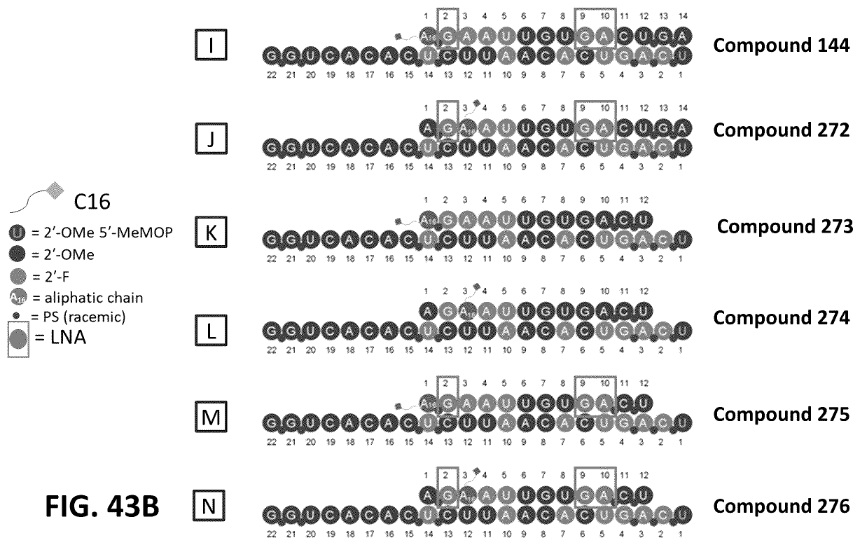

Oligonucleotide-lipid conjugates that were evaluated included those having the structures of Compounds 144, and 272-276 as shown in FIGS. 43A-43B, compared to the parent oligonucleotide-lipid conjugates of Compounds 137, 140, 217, 269, and 270. Each of Compounds 137, 140, 217, and 269-272 contain a blunt end at the 3′ terminus of the sense strand. Compounds 137, 140, 217, 269, and 270 contain a 2-nt overhang on the 5′ terminus of the antisense strand. Compounds 144, and 272-276 contain a 8-nt overhang on the 3′ terminus of the antisense strand. Compounds 144 and 272 comprise a sense strand 14 nucleotides in length. Compounds 273-276 comprise a sense strand 12 nucleotides in length.

Compounds 140 and 217 comprise locked nucleic acids on the sense strand at nucleotide positions 2, 15, and 16. Compound 270 comprises locked nucleic acids on the sense strand at nucleotide positions 8, 15, and 16. Compounds 144, 272, 275, and 276 comprise locked nucleic acids on the sense strand at the nucleotide positions 2, 9, and 10.

Compounds 137, 140, 217, 269, and 270 comprise phosphorothioate linkages between positions: 1 and 2, 18 and 19, and 19 and 20 of the sense strand. Compounds 144 and 272 comprise phosphorothioate linkages between positions: 1 and 2, 12 and 13, and 13 and 14, of the sense strand. Compounds 273-276 comprise phosphorothioate linkages between positions: 1 and 2, 11 and 12, and 12 and 13, of the sense strand. Compounds 137 and 140 comprise phosphorothioate linkages between positions 1 and 2, 2 and 3, 3 and 4, 20 and 21, and 21 and 22, of the antisense strand. Compounds 217, and 269-276 comprise phosphorothioate linkages between positions 1 and 2, 2 and 3, 3 and 4, 13 and 14, 14 and 15, 20 and 21, and 21 and 22, of the antisense strand.

The compounds comprise a C16 lipid conjugated at a different position of the sense strand as further described below:
(i) Compounds 137, 140, 217, 269-270, 144, 273, and 275 comprise a C16 lipid at position 1.
(ii) Compounds 272, 274, and 276, comprise a C16 lipid at position 3.

To evaluate the RNAi oligonucleotide-lipid conjugates, 6 to 10 week old C57BL6 mice were given a single intrathecal (i.t.) injection of 500 μg oligonucleotide-lipid conjugate formulated in artificial cerebrospinal fluid (aCSF). The RNAi oligonucleotide-lipid conjugate administered to each animal group is outlined in Table 25. Control mice received artificial cerebrospinal fluid (aCSF) only.

TABLE 25
TUBB3 RNAi oligonucleotides administered to animals

| | Sense | | Antisense | |
|---|---|---|---|---|
| Compound | SEQ ID NOS | | SEQ ID NOS | |
| ID | Unmod | Mod | Unmod | Mod |

Controls

| | | | | | |
|---|---|---|---|---|---|
| | aCSF | — | — | — | — |
| B | 137 | 372 | 386 | 324 | 352 |
| C | 140 | 372 | 541 | 324 | 352 |
| D | 217 | 372 | 541 | 324 | 533 |
| E | 269 | 372 | 386 | 324 | 533 |
| F | 270 | 372 | 723 | 324 | 533 |

P-6 LNA

| | | | | | |
|---|---|---|---|---|---|
| I | 144 | 537 | 545 | 324 | 533 |
| J | 272 | 537 | 724 | 324 | 533 |

Dual Truncation

| | | | | | |
|---|---|---|---|---|---|
| K | 273 | 638 | 725 | 324 | 533 |
| L | 274 | 638 | 726 | 324 | 533 |

Dual Truncation with LNA

| | | | | | |
|---|---|---|---|---|---|
| M | 275 | 638 | 727 | 324 | 533 |
| N | 276 | 638 | 728 | 324 | 533 |

Target knockdown was assessed 28 days post-injection. RNA was extracted from CNS tissue and murine TUBB3 mRNA levels were determined as described in Example 2.

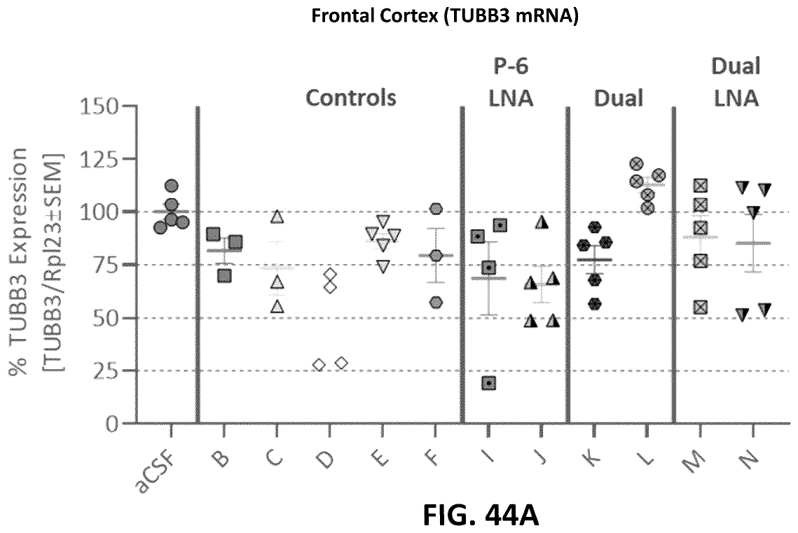

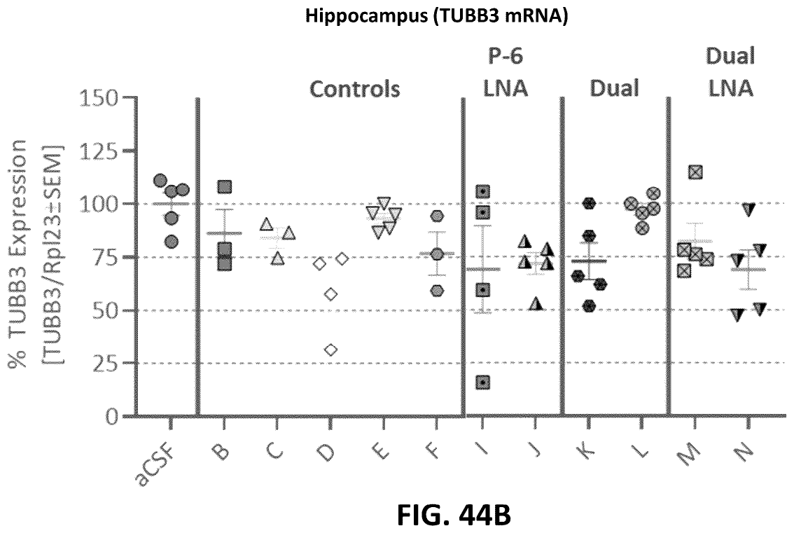

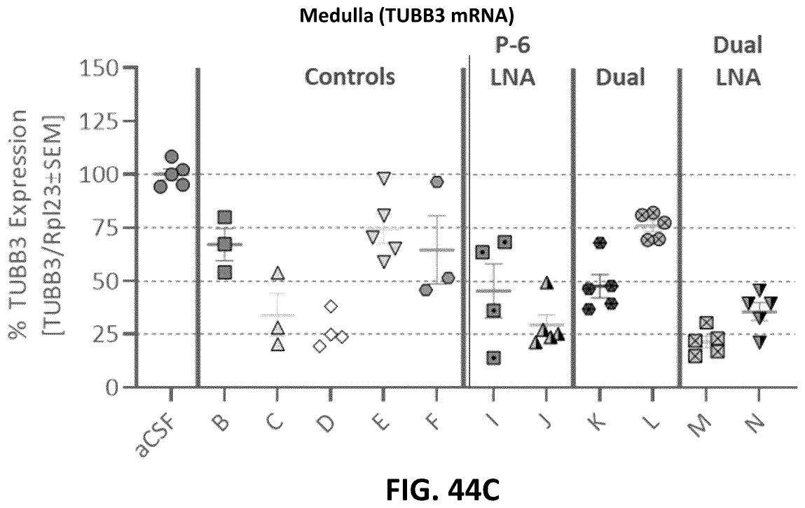

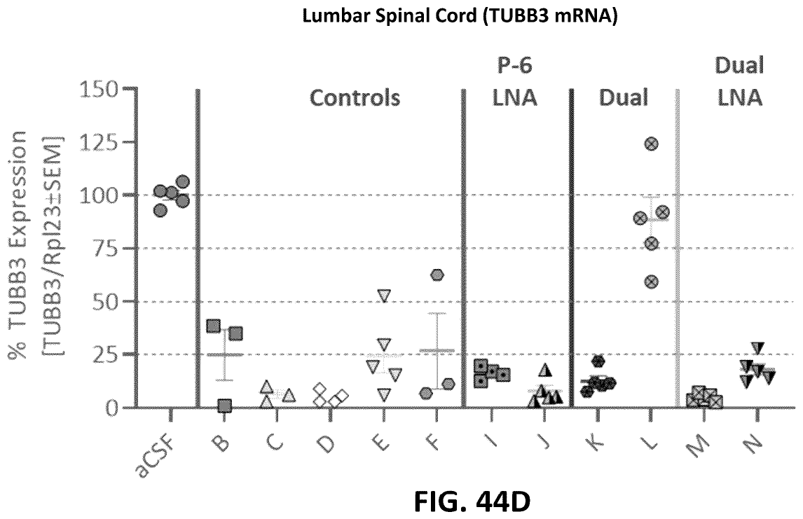

Percent TUBB3 mRNA expression remaining in the frontal cortex, hippocampus, medulla, and lumbar spinal cord as measured for each treatment group is respectively shown in FIGS. 44A-44D. Silencing of TUBB3 mRNA expression was observed in all tissues evaluated for most treatment groups. Silencing of TUBB3 mRNA expression was observed in the lumber spinal cord and medulla with all RNAi oligonucleotide-lipid conjugates tested. Addition of locked nucleic acids increased reduction of TUBB3 inhibition. Further, conjugation of a lipid at the 5′ terminal nucleotide of the sense strand provided enhanced knockdown across tissues. Together this data demonstrates the ability of RNAi oligonucleotide-lipid conjugates having truncated sense strands and/or LNAs inhibition of neuronal targets in various regions and tissues of the CNS.

### Example 26: Impact of 3′ Sense Strand Truncation for Inhibition of Target Gene in Tissue Outside the Central Nervous Tissue

RNAi oligonucleotide-lipid conjugates were evaluated to determine the effect of 3′ truncations on the sense strand. Each oligonucleotide-lipid conjugate tested comprised an antisense strand having a region of complementarity to mRNA encoding ALDH2.

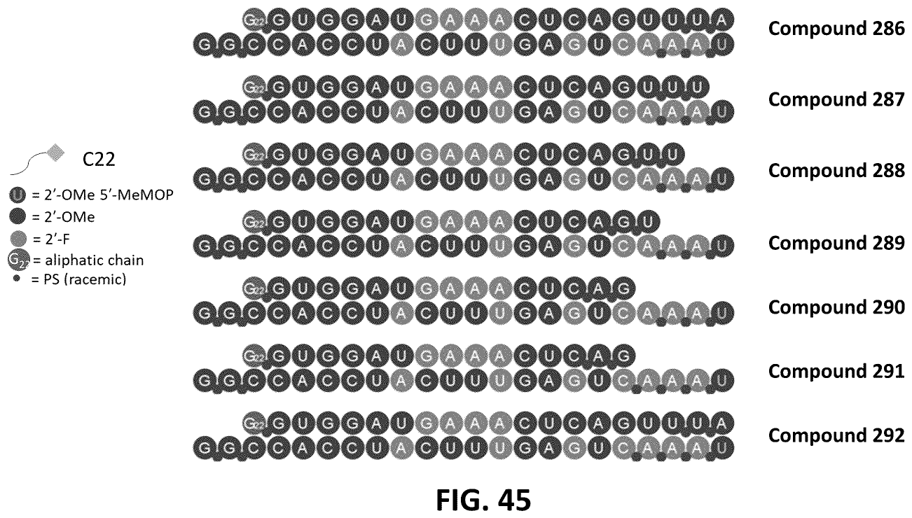

Oligonucleotide-lipid conjugates that were evaluated included those having the structures of Compounds 287-292 as shown in FIG. 45, compared to the parent oligonucleotide-lipid conjugates of Compounds 286. Each of Compounds 286-292 contain a a 2-nt overhang on the 5′ terminus of the antisense strand. Compound 287 contains a 1-nt overhang on the 3′ terminus of the antisense strand. Compound 288 contains a 2-nt overhang on the 3′ terminus of the antisense strand. Compound 289 contains a 3-nt overhang on the 3′ terminus of the antisense strand. Compounds 290 and 291 contain a 4-nt overhang on the 3′ terminus of the antisense strand.

Compounds 286 and 292 comprise phosphorothioate linkages between positions: 1 and 2, 18 and 19, and 19 and 20 of the sense strand. Compound 287 comprises phosphorothioate linkages between positions: 1 and 2, 17 and 18, and 18 and 19 of the sense strand. Compound 288 comprises phosphorothioate linkages between positions: 1 and 2, 16 and 17, and 17 and 18, of the sense strand. Compound 289 comprises phosphorothioate linkages between positions 1 and 2, 15 and 16, and 16 and 17 of the sense strand. Compounds 290 and 291 comprise phosphorothioate linkages between positions 1 and 2, 14 and 15, and 15 and 16 of the sense strand. Compounds 286-290 comprise phosphorothioate linkages between positions 1 and 2, 2 and 3, 3 and 4, 20 and 21, and 21 and 22, of the antisense strand. Compounds 291 and 292 comprise phosphorothioate linkages between positions 1 and 2, 2 and 3, 3 and 4, 4 and 5, 20 and 21, and 21 and 22, of the antisense strand.

Compounds 286-292 comprise a C22 lipid conjugated to position 1 of the sense strand.

To evaluate the RNAi oligonucleotide-lipid conjugates, 6 to 10 week old C57BL6 mice were given a single 15 mg/kg subcutaneous (s.c.) injection of oligonucleotide-lipid conjugate formulated in phosphate buffered saline (PBS). The RNAi oligonucleotide-lipid conjugate administered to each animal group is outlined in Table 26. Control mice received PBS only.

TABLE 26
GFAP RNAi oligonucleotides administered to animals

| Compound | Sense SEQ ID NOS | | Antisense SEQ ID NOS | |
|---|---|---|---|---|
| ID | Unmod | Mod | Unmod | Mod |

Controls

| | | | | | |
|---|---|---|---|---|---|
| | PBS | — | — | — | — |
| A | 286 | 742 | 747 | 205 | 752 |
| B | 287 | 743 | 748 | 205 | 752 |
| C | 288 | 744 | 749 | 205 | 752 |
| D | 289 | 745 | 750 | 205 | 752 |
| E | 290 | 746 | 751 | 205 | 752 |
| F | 291 | 746 | 751 | 205 | 753 |
| G | 292 | 742 | 747 | 205 | 753 |

Target knockdown was assessed 13 days post-injection. RNA was extracted from liver, quadricep, heart, and gonadal white adipose tissue (gWAT), and murine ALDH2 mRNA levels were determined as described in Example 11.

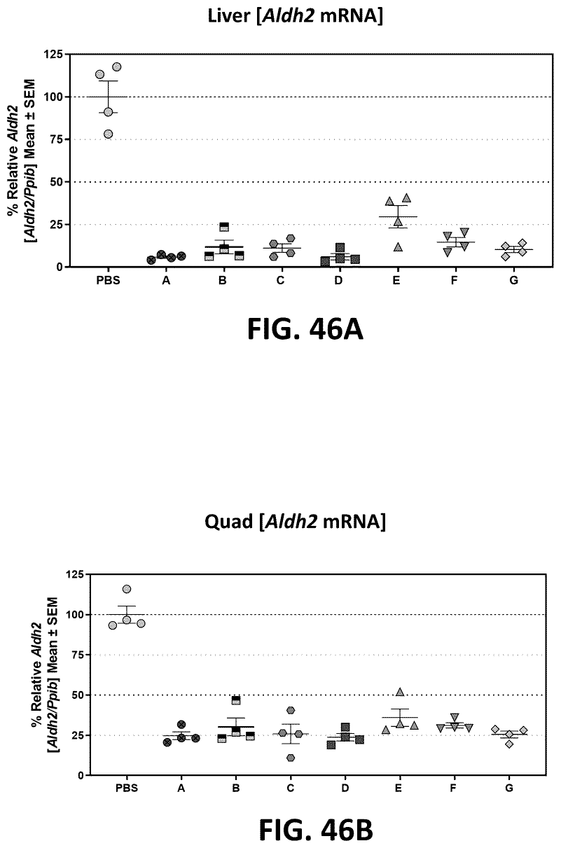

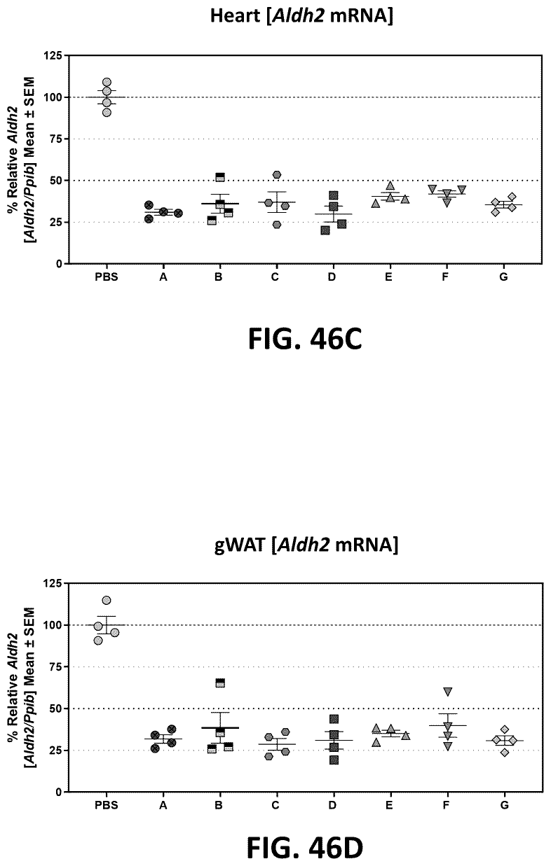

Percent ALDH2 mRNA expression remaining in the liver, quadricep, heart, and gWAT as measured for each treatment group is respectively shown in FIGS. 46A-46D. Silencing of ALDH2 mRNA expression was observed in all tissues. Specifically, 3′ truncations of the sense strand were capable of inhibiting gene expression across the various tissues. Together, this data demonstrates the ability of truncated molecules to reduce gene expression in tissues outside the CNS.

### Example 27: Truncated Sense Strand Oligonucleotides with 5′ and 3′ Overhangs for Inhibition of Oligodendrocyte Target Gene in the CNS

RNAi oligonucleotides with 5′ and/or 3′ sense strand truncations were evaluated for their ability to reduce oligodendrocyte target expression in the CNS. Each oligonucleotide-lipid conjugate tested comprised an antisense strand having a region of complementarity to mRNA encoding UGT8.

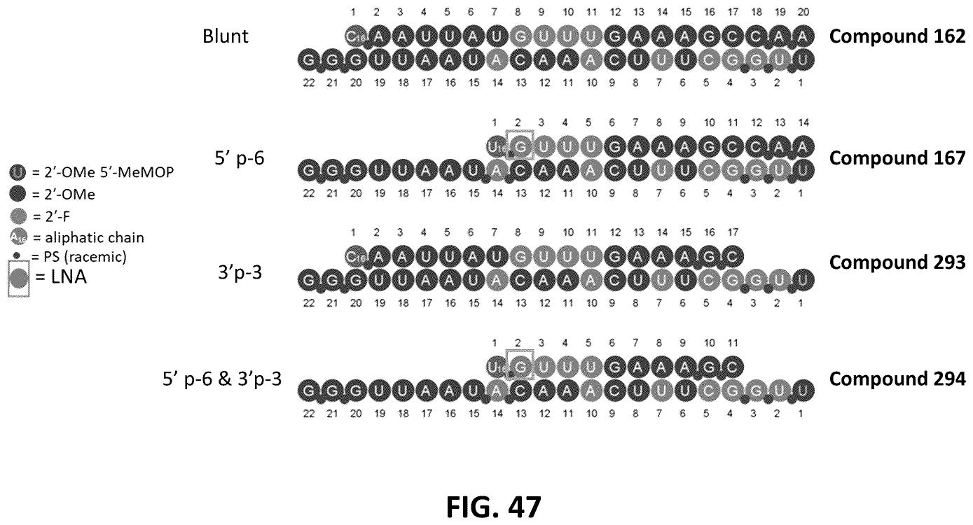

Oligonucleotide-lipid conjugates that were evaluated included those having the structures of Compounds 167, 293, and 294 as shown in FIG. 47, compared to the parent oligonucleotide-lipid conjugate of Compound 162. Each of compounds 162, 167, 293, and 294 comprise a C16 lipid at position 1 of the sense strand. Each of Compounds 162 and 167 contain a blunt end at the 3′ terminus of the sense strand. Compounds 293 and 294 contain a 3-nt overhand on the 5′ terminus of the antisense strand. Compounds 167 and 294 contain an 8-nt overhand on the 3′ terminus of the antisense strand.

Compounds 162 comprises a phosphorothioate linkages between positions: 1 and 2, 18 and 19, and 19 and 20 of the sense strand. Compound 167 comprises phosphorothioate linkages between positions: 1 and 2, 12 and 13, and 13 and 14, of the sense strand. Compound 293 comprises phosphorothioate linkages between positions: 1 and 2, 15 and 16, and 16 and 17, of the sense strand. Compound 294 comprises phosphorothioate linkages between positions: 1 and 2, 9 and 10, and 10 and 11, of the sense strand. Compounds 162 and 293 comprise phosphorothioate linkages between positions 1 and 2, 2 and 3, 3 and 4, 20 and 21, and 21 and 22, of the antisense strand. Compounds 167 and 294 comprise phosphorothioate linkages between positions 1 and 2, 2 and 3, 3 and 4, 13 and 14, 14 and 15, 20 and 21, and 21 and 22, of the antisense strand.

To evaluate the RNAi oligonucleotide-lipid conjugates, 6 to 10 week old C57BL6 mice were given a single intrathecal (i.t.) injection of 300 μg oligonucleotide-lipid conjugate formulated in artificial cerebrospinal fluid (aCSF). The RNAi oligonucleotide-lipid conjugate administered to each animal group is outlined in Table 27. Control mice received artificial cerebrospinal fluid (aCSF) only.

TABLE 27
Truncated RNAi Oligonucleotides

| | | Sense | | Antisense | |
|---|---|---|---|---|---|
| | | SEQ ID NOS | | SEQ ID NOs | |
| Compound | Oligonucleotide | Unmod | Mod | Unmod | Mod |
| | aCSF | — | — | — | — |
| 162 | 2-nt 5′ Truncated | 565 | 567 | 577 | 578 |
| 167 | 8-nt 5′ Truncated Sense | 566 | 572 | 577 | 579 |
| | Strand | | | | |
| 293 | 2-nt 5′ Truncated and | 738 | 740 | 577 | 578 |
| | 3-nt 3′ Truncated | | | | |
| 294 | 8-nt 5′ Truncated and | 739 | 741 | 577 | 579 |
| | 3-nt 3′ Truncated | | | | |

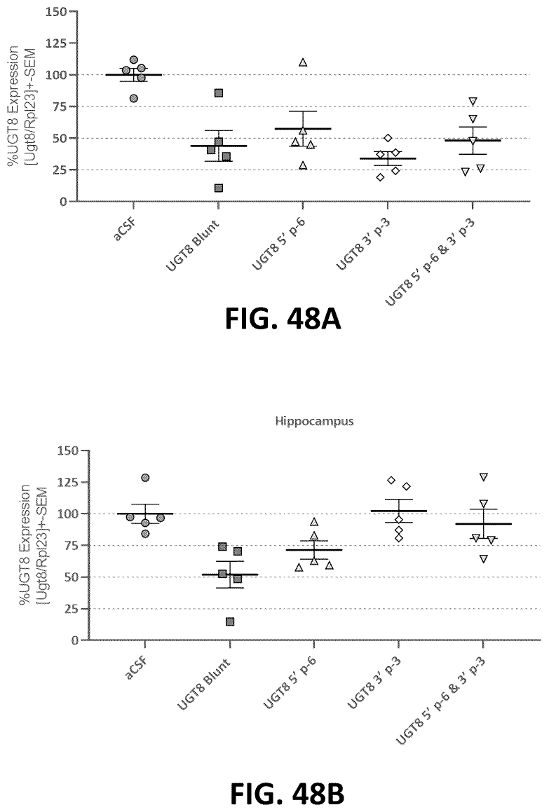

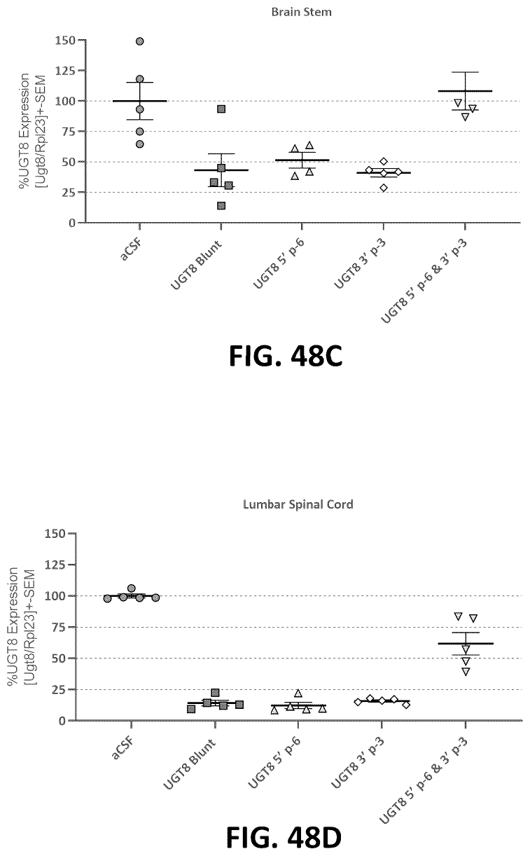

Target knockdown was assessed 28 days post-injection. RNA was extracted from CNS tissue and murine UGT8 mRNA levels were determined as described in Example 2. Percent UGT8 mRNA expression remaining in the frontal cortex, hippocampus, brain stem, and lumbar spinal cord as measured for each treatment group is respectively shown in FIGS. 48A-48D. Silencing of UGT8 mRNA expression was observed in all tissues. Specifically, truncations of the sense strand greater than 2 nucleotides performed similarly to the blunt-end oligonucleotide with a 2-nt truncation (Compound 162) throughout the CNS. Together, this data demonstrates the ability of truncated molecules to reduce oligodendrocyte target genes in the CNS.

### Example 28: Preparation of RNAi Oligonucleotides

### Oligonucleotide Synthesis and Purification

The oligonucleotides (RNAi oligonucleotides) described in the foregoing Examples were chemically synthesized using methods described herein. Generally, RNAi oligonucleotides were synthesized using solid phase oligonucleotide synthesis methods as described for 19-23mer RNAi oligonucleotides (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g., Hughes and Ellington (2017) COLD SPRING HARB PERSPECT BIOL. 9(1):a023812; Beaucage S. L., Caruthers M. H. STUDIES ON NUCLEOTIDE CHEMISTRY V: Deoxynucleoside Phosphoramidites-A New Class of Key Intermediates for Deoxypolynucleotide Synthesis, TETRAHEDRON LETT. 1981; 22:1859-62. doi: 10.1016/S0040-4039(01)90461-7); PCT application No. PCT/US2021/42469 (each of which is incorporated herein by this reference)). RNAi oligonucleotides having a 19mer core sequence were formatted into constructs having a 36mer sense strand and a 22mer antisense strand to allow for processing by the RNAi machinery. The 19mer core sequence is complementary to a region in the GFAP mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84) and the phosphoramidite synthesis as shown below:

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

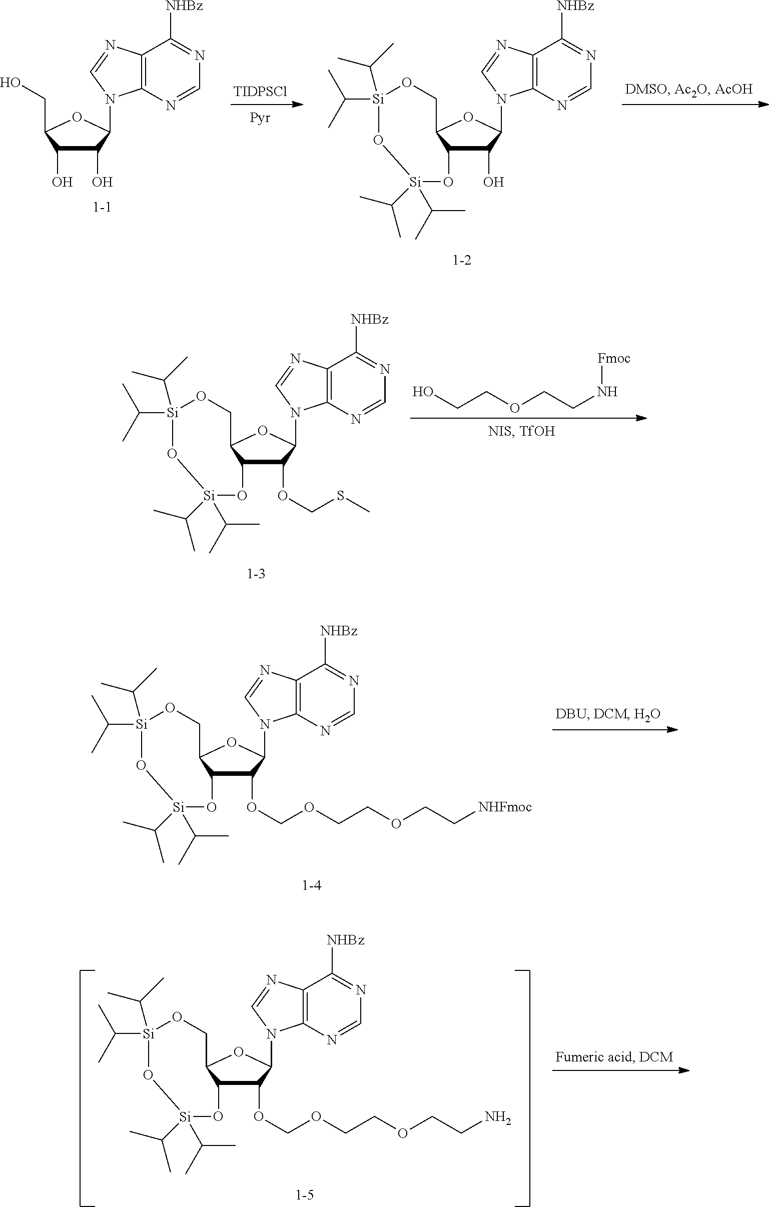

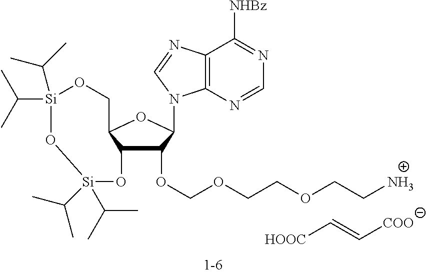

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHZ, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

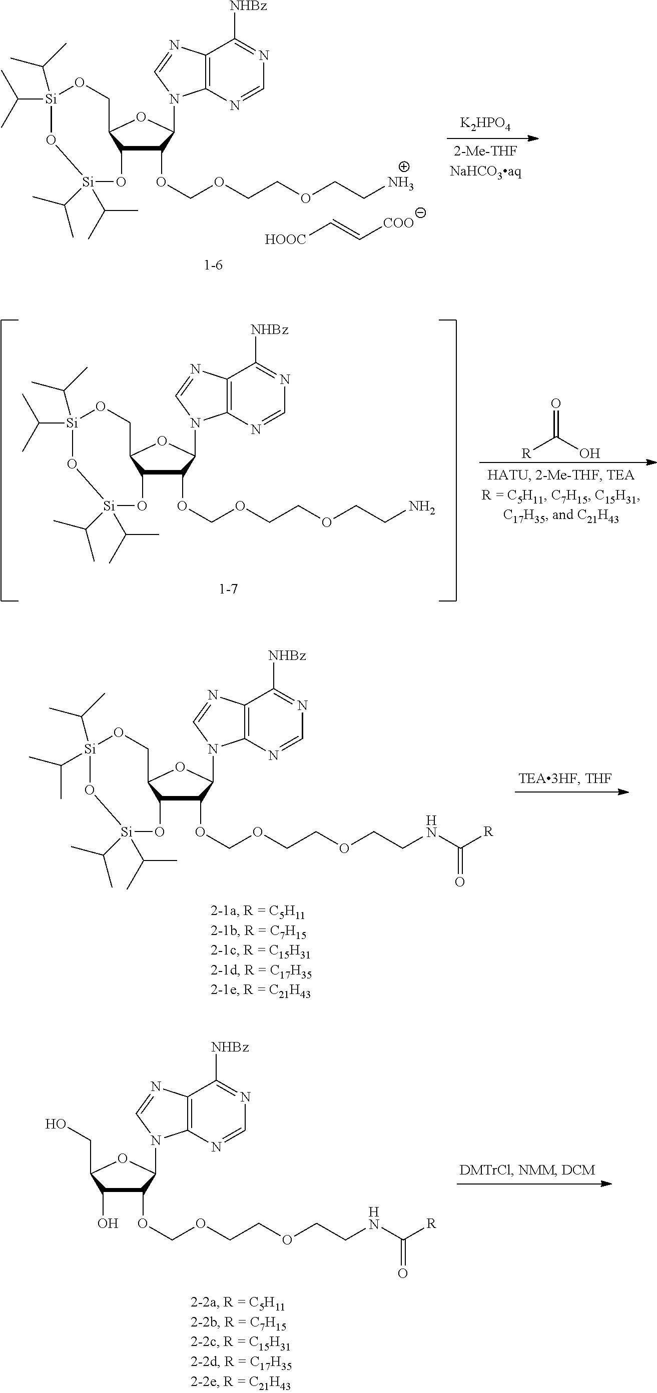

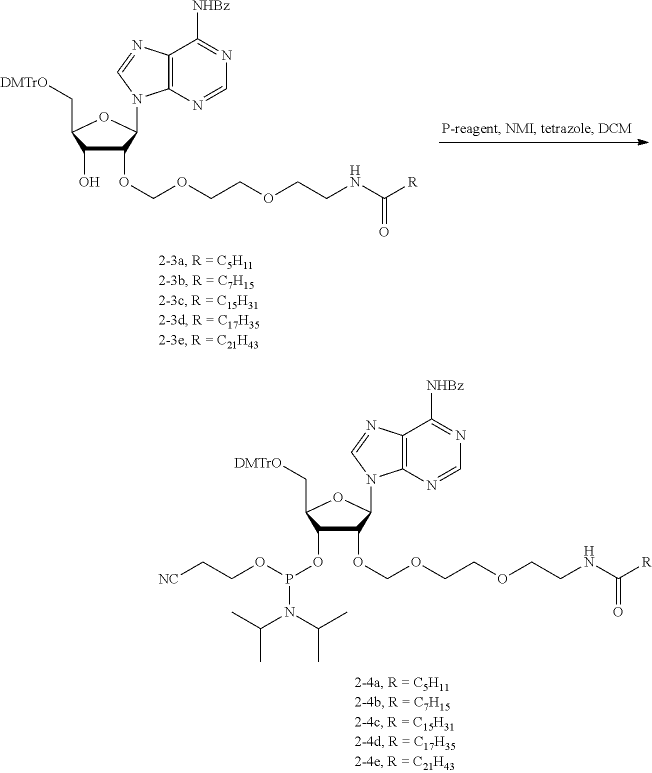

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHZ, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHZ, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHZ, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

The synthesis methods described herein are used to generate the lipid-conjugated oligonucleotides described in Examples 31, 32, 34, and 35.

### Example 29: GalNAc-Conjugated GFAP RNAi Oligonucleotides Inhibit Mouse Gfap In Vivo in a Concentration Dependent Manner Via Intrathecal and Intracerebroventricular Administration

Glial fibrillary acidic protein (GFAP) encodes intermediate filament proteins primarily found in astrocytes of the CNS. Sufficient knockdown of GFAP indicates ability of oligonucleotides described herein to reduce expression of target genes expressed in astrocytes. To evaluate the ability of RNAi oligonucleotides to reduce Gfap expression in vivo, oligonucleotides synthesized as described in Example 28 were used to generate double-stranded RNAi oligonucleotides comprising a nicked tetraloop GalNAc-conjugated structure (referred to herein as “GalNAc-conjugated GFAP oligonucleotides” or “GalNAc-GFAP oligonucleotides”) having a 36-mer passenger strand and a 22-mer guide strand. Further, the nucleotide sequences comprising the passenger strand and guide strand have a distinct pattern of modified nucleotides and phosphorothioate linkages. Three of the nucleotides comprising the tetraloop were each conjugated to a GalNAc moiety (CAS #14131-60-3). The modification pattern of each strand is illustrated below:

Sense Strand:
5′[mXs][mX][mX][mX][X][mX][mX][fX][fX][fX][fX][mX]
mX][mX][mX][mX][mX][mX][mX][mX][X][mX][mX][mX][X]
[mX][mX][ademX-GalNAc][ademX-GalNAc][ademX-
GalNAc][mX][mX][mX][mX][mX][mX]3′

### Hybridized to:

Antisense Strand:
5′ [MePhosphonate-4O-mXs][fXs][fXs][fX][fX][mX]
[fX][mX][mX][fX][mX][mX][mX][fX][X][mX][mX][X][mX]
[mXs][mXs][mX] 3′

TABLE 28
Modification key:

| Symbol | Modification/linkage |
|---|---|
| [MePhosphonate- | 4′-O-monomethylphosphonate-2′-O-methyl |
| 4O-mX] | modified nucleotide |
| [ademX-GalNAc] | GalNAc attached to a nucleotide |
| [mXs] | 2′-O-methyl modified nucleotide with a |
| | phosphorothioate linkage to the |
| | neighboring nucleotide |
| [fXs] | 2′-fluoro modified nucleotide with a |
| | phosphorothioate linkage to the |
| | neighboring nucleotide |
| [mX] | 2′-O-methyl modified nucleotide with |
| | phosphodiester linkages to |
| | neighboring nucleotides |
| [fX] | 2′-fluoro modified nucleotide with |
| | phosphodiester linkages to |
| | neighboring nucleotides |
| [AdemX-C16] | C16 lipid attached to anucleotide |
| [AdemX-C16s] | C16 lipid attached to a nucleotide |
| | with a phosphorothioate linkage |
| | to the neighboring nucleotide |

i. Intrathecal Administration

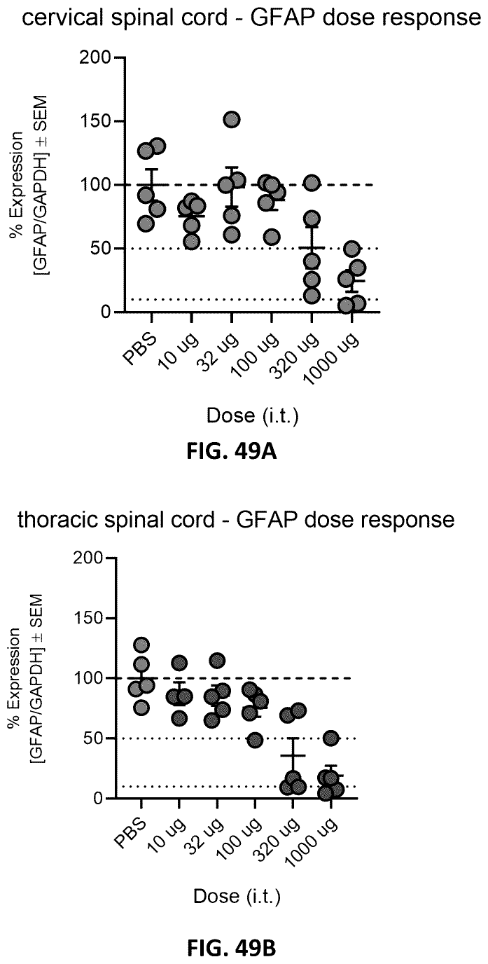

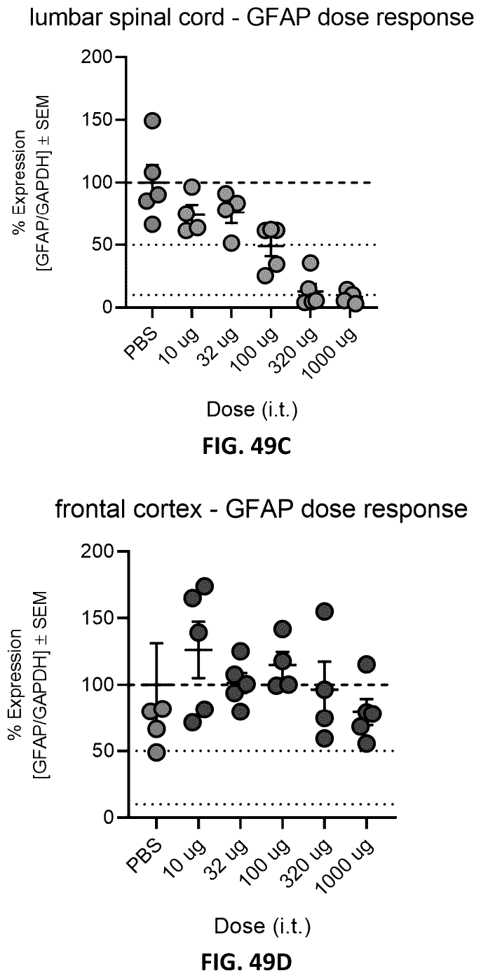

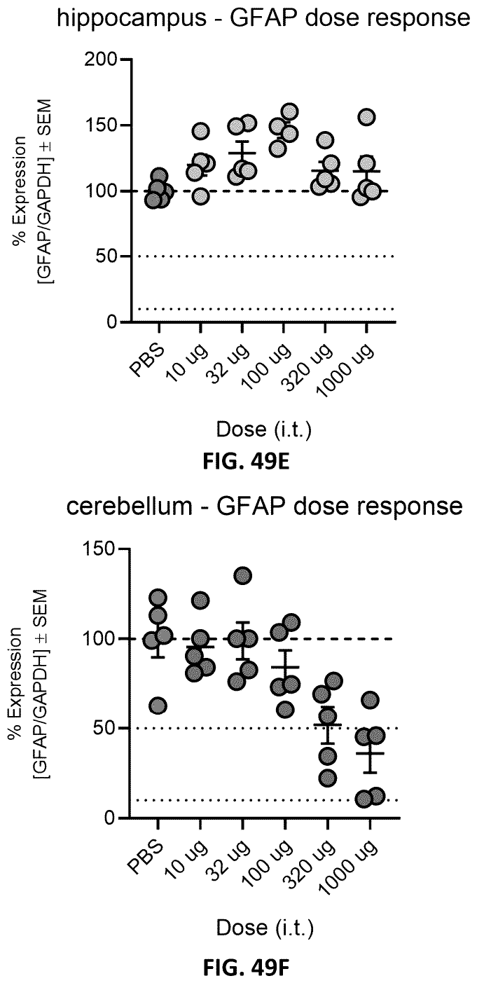

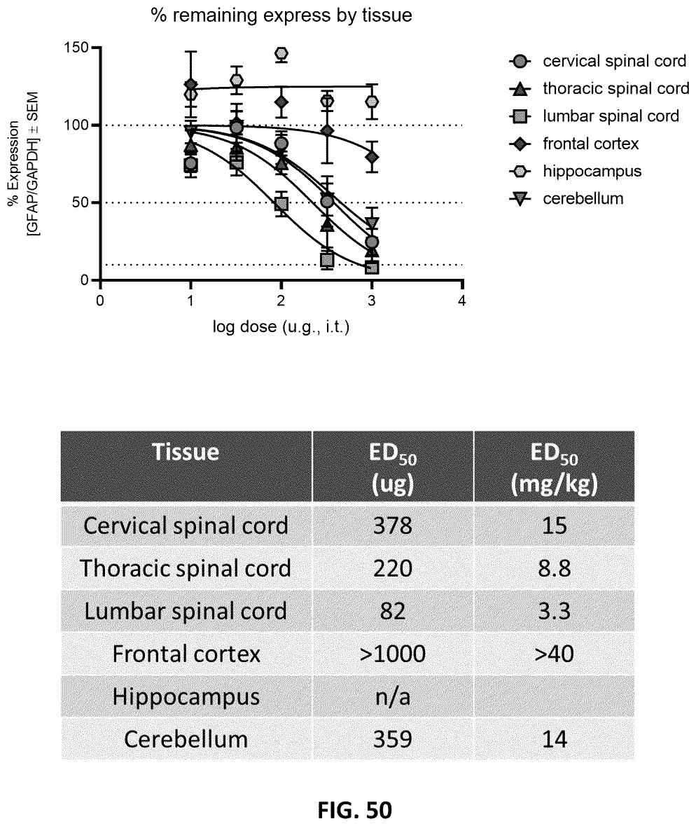

GFAP-1477, as shown in Table 29, was assessed for the ability to reduce murine Gfap in the central nervous system (CNS) via intrathecal (i.t.) administration. Specifically, mice were administered 10, 32, 100, 320, or 1000 μg of GFAP-1477 formulated in artificial cerebrospinal fluid (aCSF) via i.t. lumbar injections. Animals were sacrificed 7 days following intrathecal injection. RNA was extracted from CNS tissue to determine murine Gfap mRNA levels by qPCR (normalized to endogenous housekeeping gene Rpl23). The levels of murine Gfap mRNA were determined using PrimeTime™ qPCR Probe Assays (IDT), which consisted of a primer pair and fluorescently labeled 5′ nuclease probe specific to murine Gfap mRNA. The percentage of murine Gfap mRNA remaining in the samples from treated mice was determined using the 2^{-ΔΔCt} (“delta-delta Ct”) method (Livak and Schmittgen (2001) METHODS 25:402-408). Reduction of Gfap expression was observed in a dose dependent manner across tissues of the CNS. Specifically, Gfap expression was reduced by about 50% or greater in samples from the cervical spinal cord, thoracic spinal cord, lumbar spinal cord, and cerebellum as shown in FIGS. 49A, 49B, 49C, and 49F, respectively. Less reduction of Gfap expression was observed in the frontal cortex and hippocampus as shown in FIGS. 49D and 49E, respectively. The average percent (%) of mRNA remaining is shown in FIG. 50, along with the ED₅₀ values determined for each region of the brain based on the percent mRNA remaining in the tissue at each concentration of oligonucleotide. Together, this data demonstrates GFAP targeting oligonucleotides having a GalNAc-conjugated tetraloop inhibit GFAP expression following i.t. administration in a dose-dependent manner in several tissues of the CNS.

TABLE 29
GalNAc-Conjugated mouse Gfap RNAi Oligonucleotide

| | Unmodified | Unmodified | Modified | Modified |
|---|---|---|---|---|
| | Sense | Antisense | Sense | Antisense |
| | Strand | strand | Strand | strand |
| GFAP-1147 | 756 | 757 | 758 | 771 |

ii. Intracerebroventricular Administration

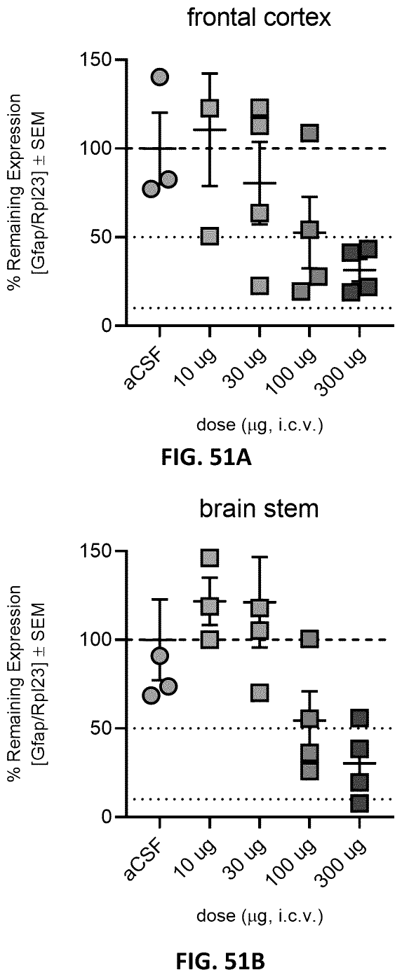

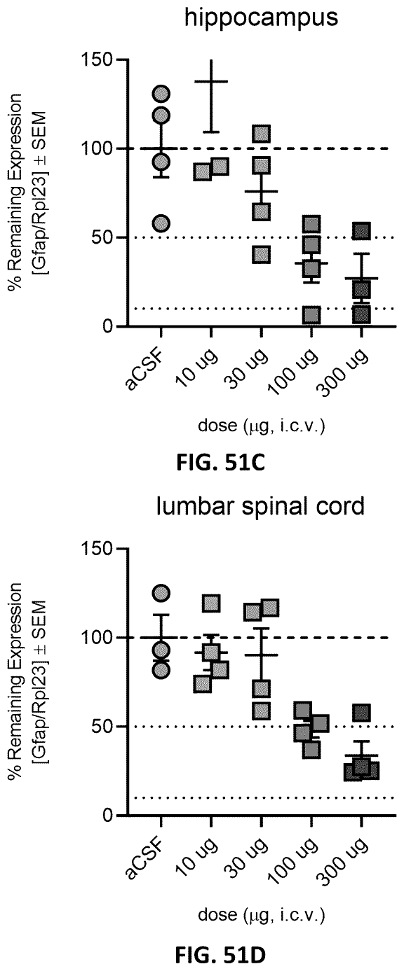

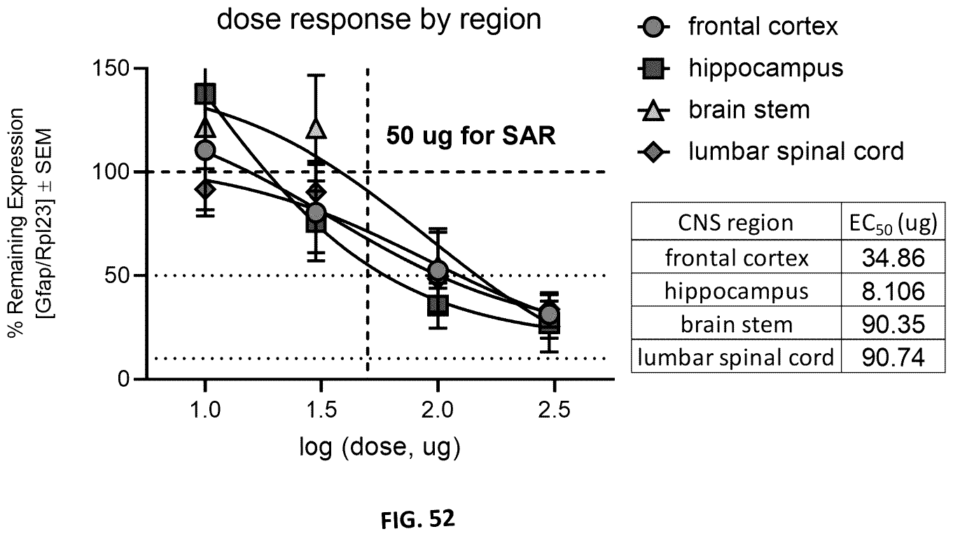

GFAP-1477 was also assessed for the ability to reduce murine Gfap in the central nervous system (CNS) via intracerebroventricular (i.c.v) administration. Specifically, mice were administered 10, 32, 100, or 300 μg of GFAP-1477 formulated in aCSF via i.c.v. Animals were sacrificed 7 days following i.c.v. injection. RNA was extracted from CNS tissue as described above. Reduction of Gfap expression was observed in a dose-dependent manner across tissues of the CNS. Specifically, Gfap expression was reduced by about 50% or greater in samples of the frontal cortex, brain stem, hippocampus, and lumbar spinal cord as shown in FIGS. 51A-51D, respectively. The average percent (%) of mRNA remaining is shown in FIG. 52, along with the ED₅₀ values determined for each region of the brain based on the percent mRNA remaining in the tissue at each concentration of oligonucleotide. Together, this data demonstrates GFAP targeting oligonucleotides having a GalNAc-conjugated tetraloop inhibit GFAP expression in several tissues of the CNS via i.c.v administration in a dose-dependent manner.

### Example 30: Synthesis of Tetraloop RNAi Oligonucleotide-Lipid Conjugates

Lipid-conjugated tetraloop oligonucleotides described herein can be synthesized using post-synthetic methods described in detail in PCT application No. PCT/US2021/42469. Specifically, the oligonucleotides can be synthesized using a post-synthetic conjugation approach such as that depicted below.

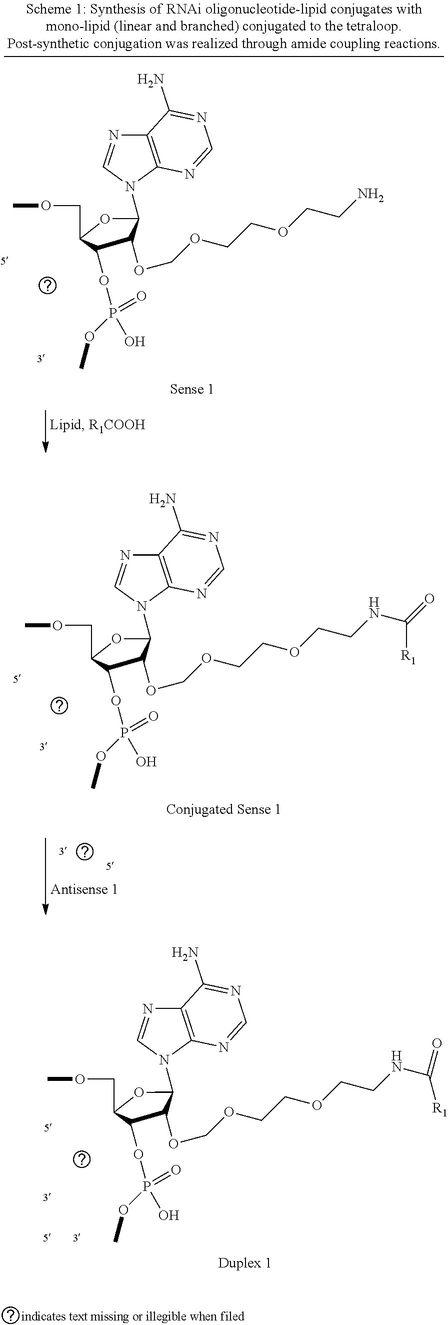

R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1. Exemplary R₁ structures are provided below:

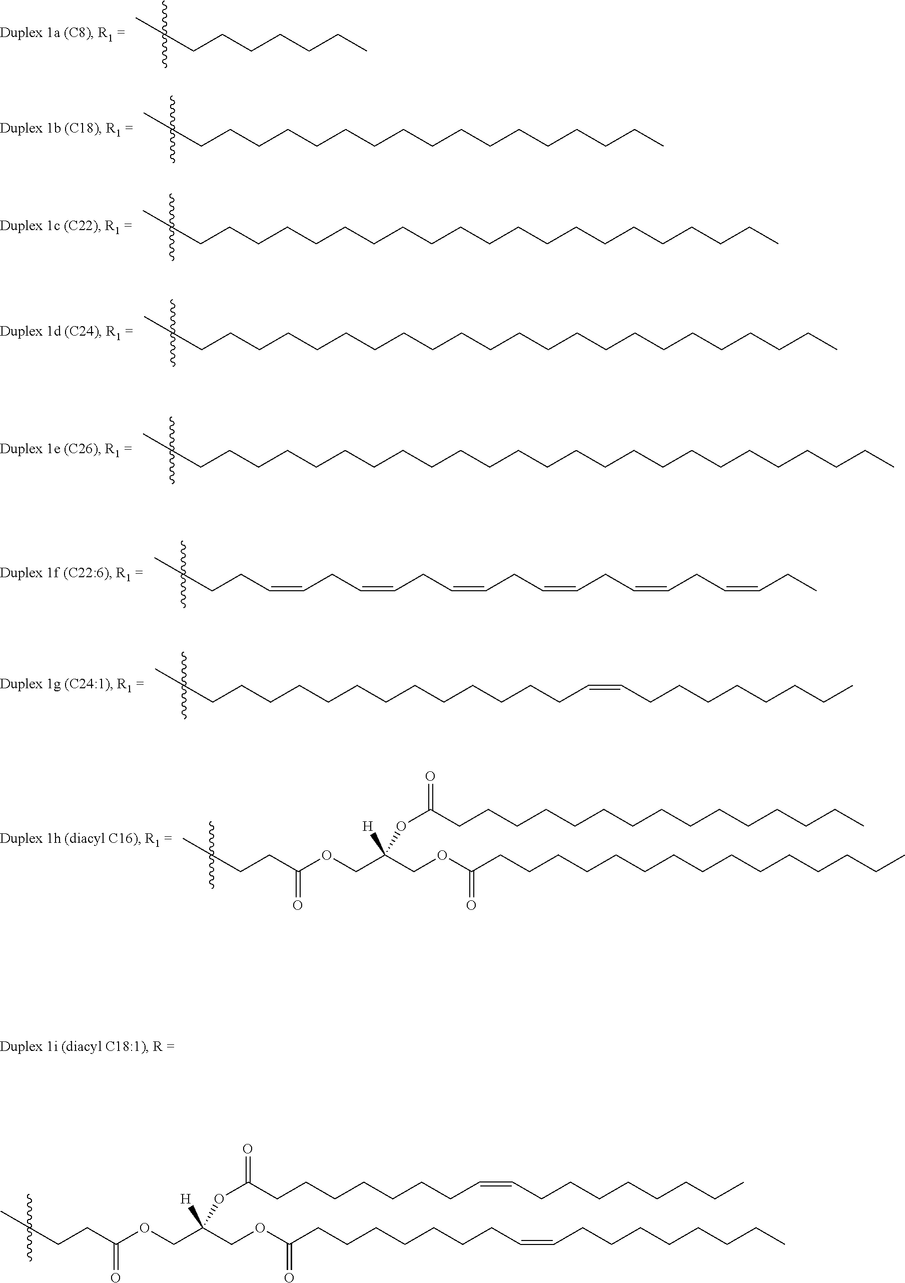

Synthesis Sense 1 and Antisense 1 were prepared by solid-phase synthesis. Synthesis of Conjugated Sense 1a-1i.

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

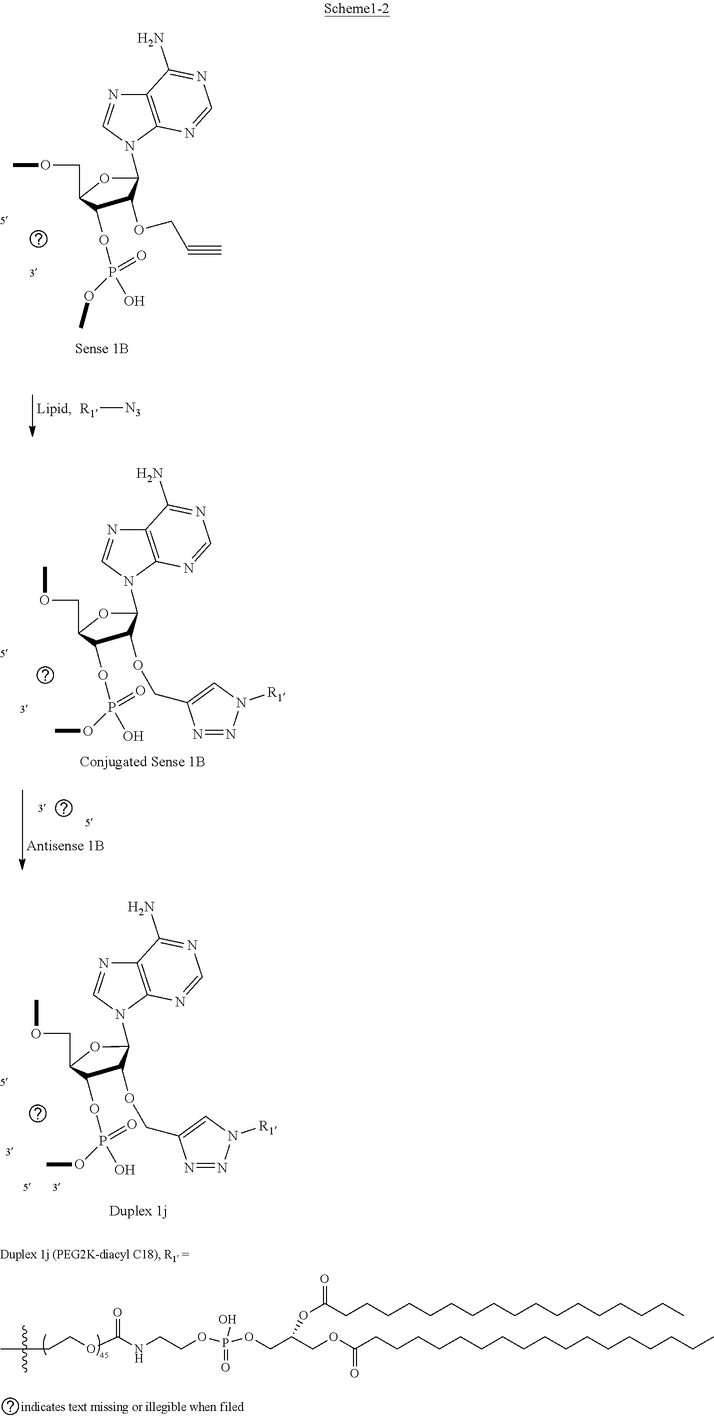

Sense 1B and Antisense 1B were prepared by solid-phase synthesis.

### Synthesis of Conjugated Sense 1j.

In Eppendorf tube 1, a solution of oligo (10.00 mg, 0.8 umol) in a 3:1 mixture of DMA/H₂O (0.5 mL) was treated with the lipid linker azide (11.26 mg, 4 umol). In Eppendorf tube 2, CuBr dimethyl sulfide (1.64 mg, 8 umol) was dissolved in ACN (0.5 mL). Both solutions were degassed for 10 min by bubbling N₂ through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase XBridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

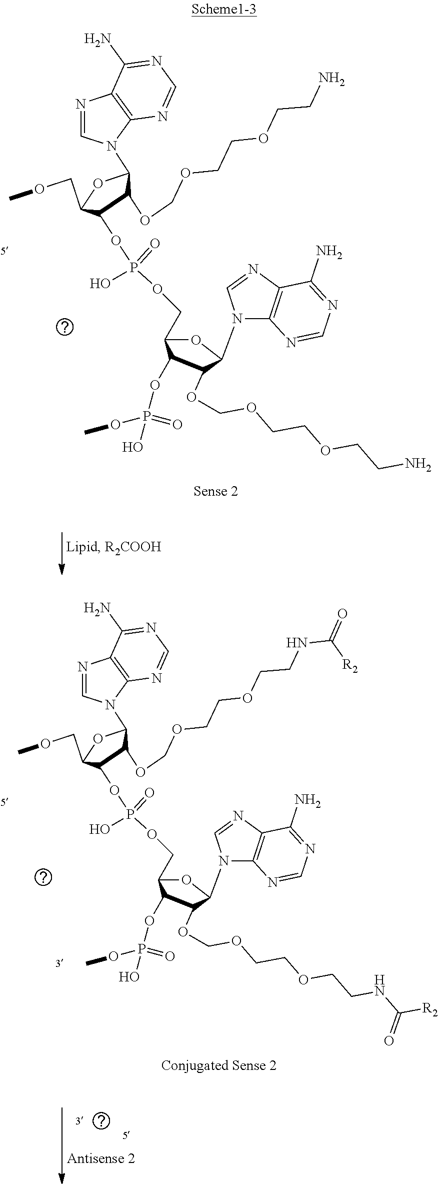

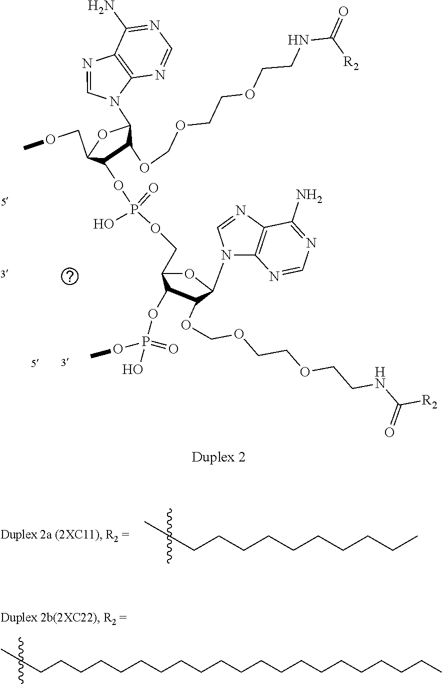

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

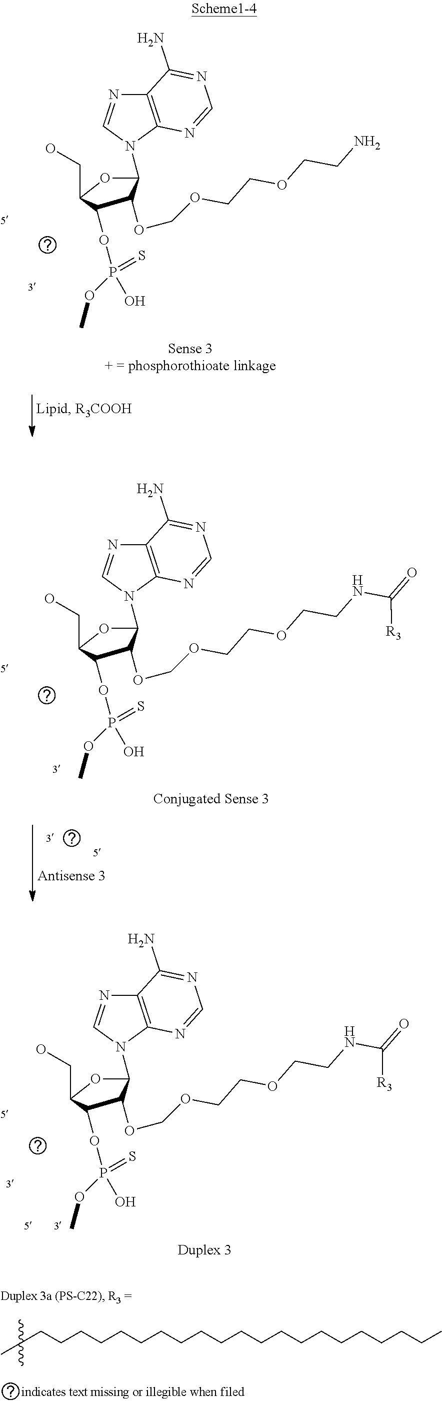

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS—C22) was prepared using the same procedures as described for the annealing of Duplex 1a (C8).

The following Scheme 1-5 depicts the synthesis of GalXC of short sense conjugated with mono-lipid using post-synthetic conjugation approach.

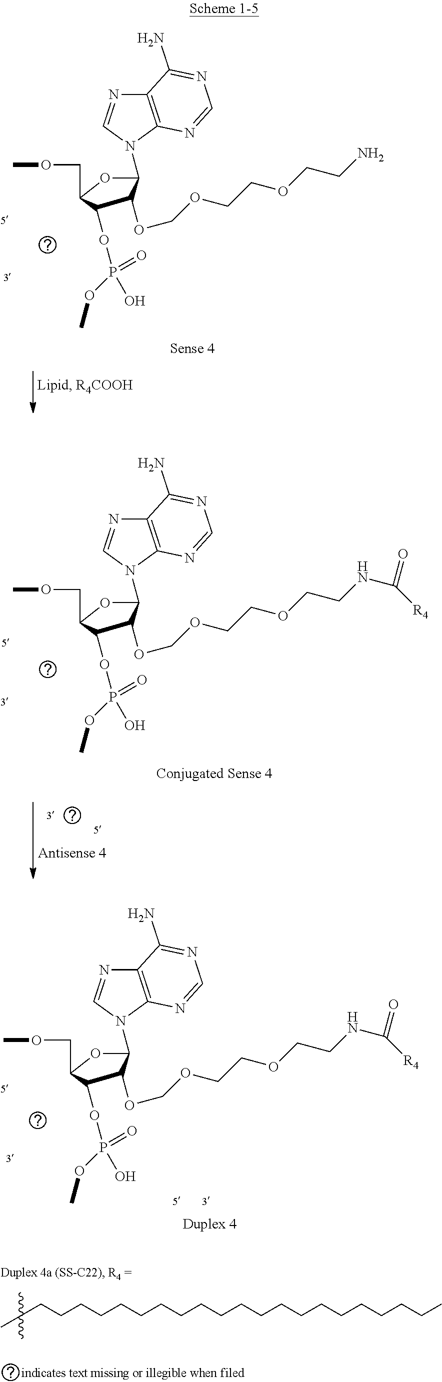

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS—C22) was prepared using the same procedures as described for the annealing of Duplex 1a (C8).

The following scheme 1-6 depicts an example of solid phase synthesis of Nicked tetraloop GalXC conjugated with lipid(s) on the loop.

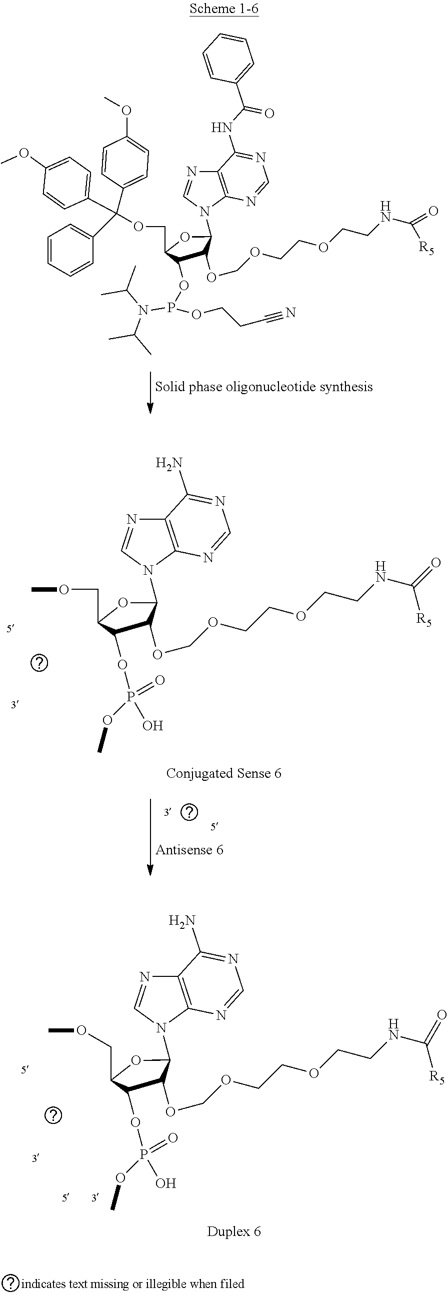

### Synthesis of Conjugated Sense 6.

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′ direction using a standard oligonucleotide synthesis protocol. 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed, and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

The following Scheme 1-7 depicts the synthesis of GalXC conjugated with mono-lipid at 5′-end using post-synthetic conjugation approach.

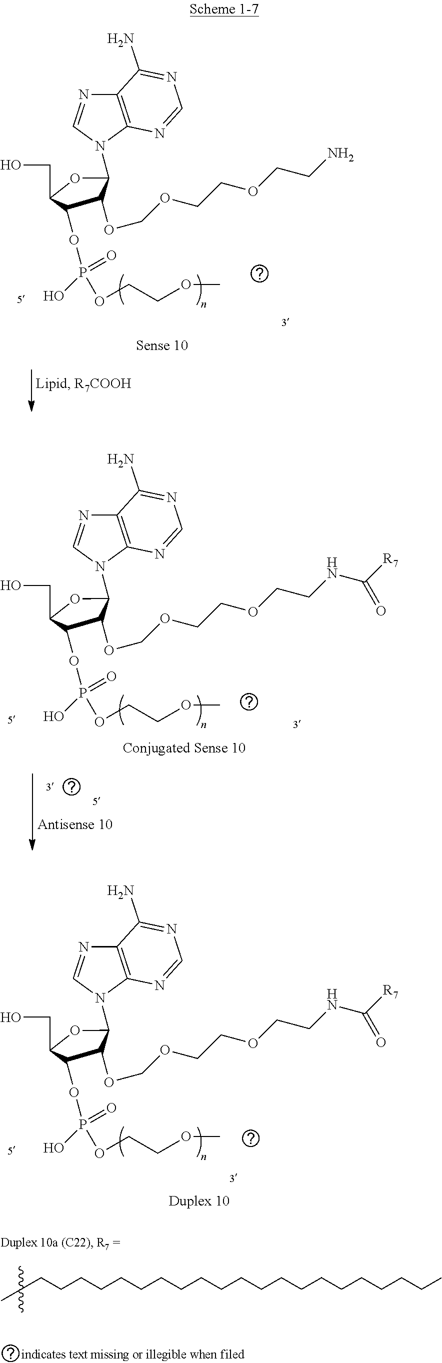

### Synthesis of Conjugated Sense 10a

Conjugated Sense 10a was obtained using the same method or a substantially similar method to the synthesis of Conjugated Sense 5.

Synthesis example of Duplex 10a

Duplex 10a was obtained using the same method or a substantially similar method to the synthesis of Duplex 5.

### Example 31: GFAP-Targeting Oligonucleotides Demonstrate Long-Term Duration of Action Via Intrathecal and Intracisterna Magna Administration

In this example, duration of action was determined using oligonucleotides with different targeting ligands. Specifically, a GalNAc-conjugated Gfap oligonucleotide as described in Example 29, and a C16-conjugated Gfap oligonucleotide generated by methods described in Example 28, the sequences of which are provided in Table 30 were administered to rats for long term-studies. A C16 lipid moiety was conjugated at position 28 of the sense strand.

### Tetraloop RNAi Oligonucleotide Modification Pattern:

Sense Strand:
5′[mXs][mX][mX][mX][mX][mX][mX][fX][fX][fX][fX]
[mX][X][mX][mX][mX][mX][mX][mX][mX][mX][mX][mX]
[mX][mX][mX][mX][ademX-C16][mX][mX][mX][mX][mX]
[mX][mX][mX]3′

### Hybridized to:

Antisense Strand:
5′[MePhosphonate-4O-mXs][fXs][fXs][fX][fX][mX]
[fX][mX][mX][fX][mX][mX][mX][fX][mX][mX][mX][mX]
[mX][mXs][mXs][mX]3′

### Modification key: Table 28

i. Intrathecal Administration

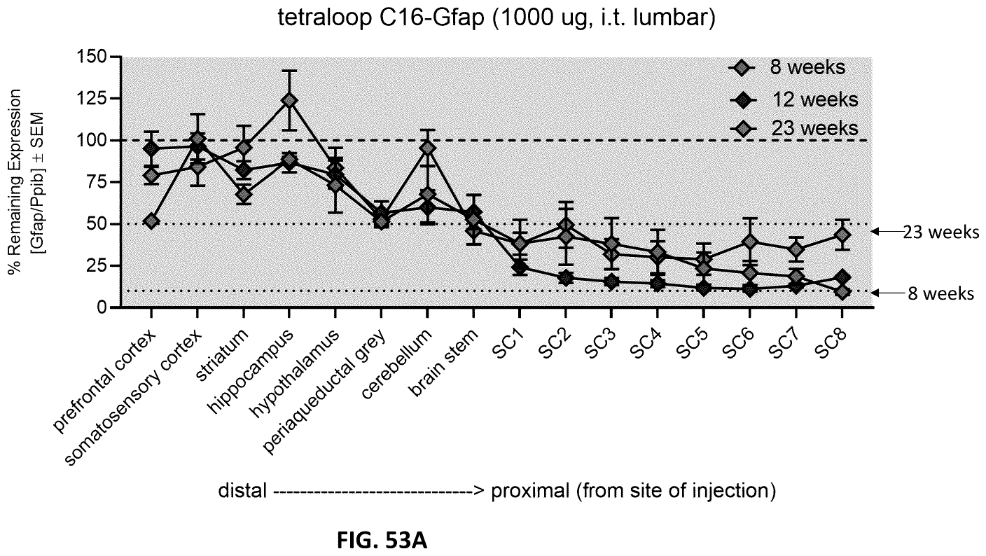

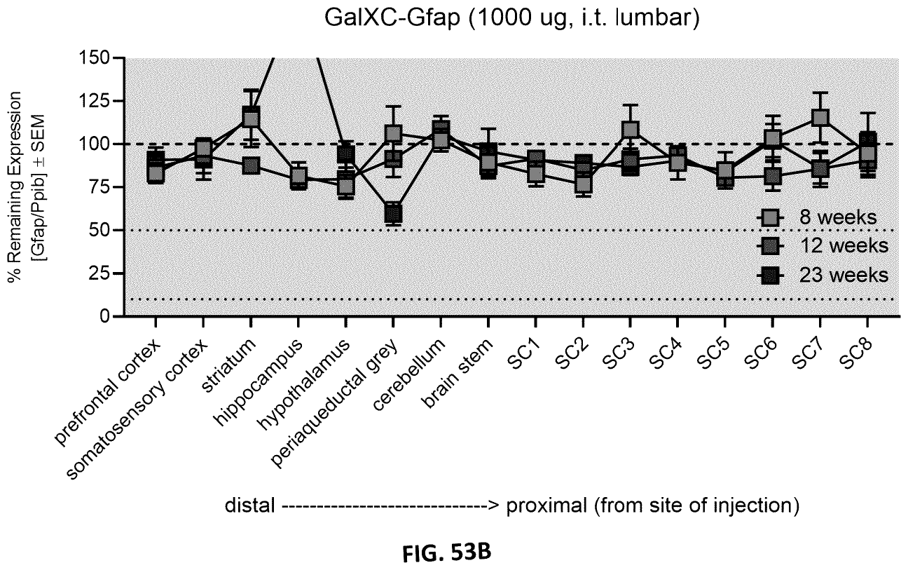

The GalNAc-conjugated and lipid-conjugated Gfap oligonucleotides described above were administered to rats via intrathecal (i.t.) injection to assess long-term duration of reduction of Gfap expression. Specifically, Sprague-Dawley rats (250 g) were administered a single 1000 μg dose of an oligonucleotide in Table 30 formulated in aCSF vis i.t. injection. Target knock-down was assessed 8, 12, and 23 weeks after i.t. injection. RNA was extracted from tissue samples of the prefrontal cortex, somatosensory cortex, striatum, hippocampus, periaqueductal grey, cerebellum, brain stem, and spinal cord (SC) segments 1-8 (SC1-SC8) to determine rat Gfap mRNA levels by qPCR as described in Example 29. The levels of murine Gfap mRNA were determined using PrimeTime™ qPCR Probe Assays (IDT), The C16-conjugated oligonucleotide demonstrated about 50% or greater reduction of Gfap expression in the brainstem and SC1-SC8 over the course of the study (i.e., at 8, 12, and 23 weeks) (FIG. 53A). In contrast, less reduction of Gfap expression was observed using the GalNAc-conjugated oligonucleotide across all sampled brain tissues (FIG. 53B). This data demonstrates the long-term sustained inhibition of astrocyte target mRNA (e.g., GFAP) in the central nervous system following a single intrathecal injection.

TABLE 30
Tetraloop-conjugated RNAi Oligonucleotides

| | | Un- | Un- | | |
|---|---|---|---|---|---|
| | | modified | modified | Modified | Modified |
| | Ligand | Sense | Antisense | Sense | Antisense |
| | Conjugate | Strand | strand | Strand | strand |
| GFAP-1147 | GalNAc | 756 | 757 | 758 | 771 |
| GFAP-1147 | C16 lipid | 756 | 757 | 768 | 771 |

ii. Intracisterna Magna Administration

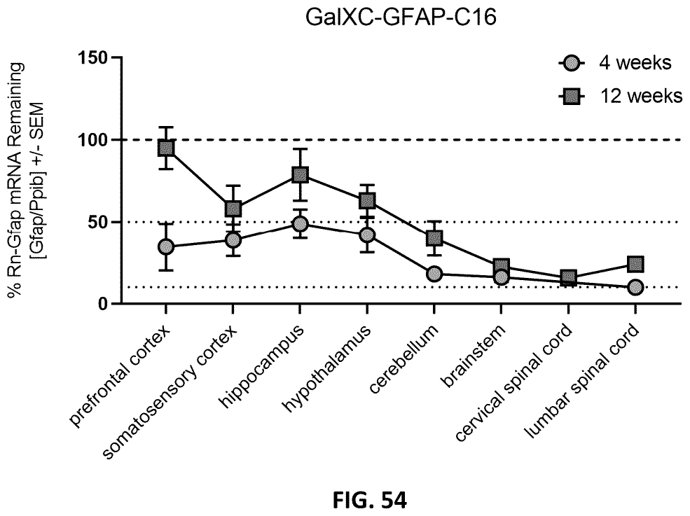

The C16-conjugated GFAP oligonucleotide described in Table 30 was administered to rats intracisterna magna (i.c.m.) injection to assess long-term duration of reduction of Gfap expression. Specifically, Sprague-Dawley rats were administered a single 1000 μg dose of oligonucleotide formulated in aCSF via i.c.m injection. Gfap expression was assessed 4 and 12 weeks after injection. RNA was extracted from tissue samples of the prefrontal cortex, somatosensory cortex, hippocampus, hypothalamus, cerebellum, brain stem, cervical spinal cord, and lumbar spinal cord to determine rat Gfap mRNA levels by qPCR as described in Example 29. About 50% or greater reduction in Gfap expression was observed in in all brain regions at 4-weeks following i.c.m. injection, whereas about 50% or greater reduction in Gfap expression was maintained in the cerebellum, brainstem, cervical spinal cord, and lumbar spinal cord out to 12 weeks following i.c.m. injection (FIG. 54).

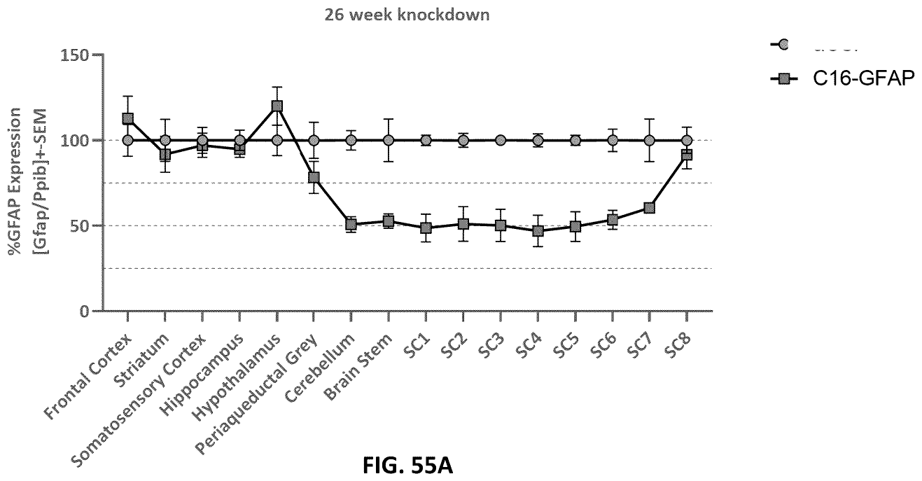

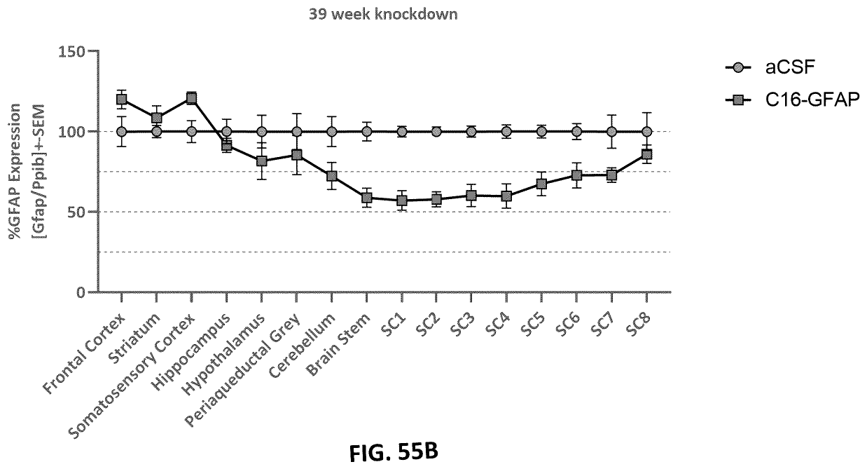

A second cohort of animals were administered the same C16 lipid-conjugated Gfap oligonucleotide in Table 30 to assess the reduction of Gfap mRNA expression out to 26 and 39 weeks. Rats were administered a single dose of the oligonucleotide as described above. RNA was extracted from tissue samples of the frontal cortex, striatum, somatosensory cortex, hippocampus, hypothalamus, periaqueductal grey, cerebellum, brain stem, and spinal cord (SC) segments 1-8 (SC1-SC8) to determine rat Gfap mRNA levels by qPCR as described in Example 29. About 50% or greater reduction of Gfap expression was observed at 26-weeks post dose in the cerebellum, brainstem, and SC1-SC5 (FIG. 55A). Out to 39 weeks, about 25-50% reduction of Gfap expression was observed in the cerebellum, brainstem, and SC1-SC7 (FIG. 55B). This data demonstrates the long-term sustained inhibition of astrocyte target mRNA (e.g., GFAP) in the central nervous system following a single intracisterna magna injection.

### Example 32: Positional Effects of Lipid Conjugation on the In Vivo Activity of Tetraloop RNAi Oligonucleotide Lipid-Conjugates to the Central Nervous System

The ability of RNAi oligonucleotide-lipid conjugates comprising a tetraloop to reduce mRNA expression in astrocytes of the CNS was evaluated in vivo. C16-conjugated Gfap oligonucleotides were generated as described in Example 28. Specifically, a C16 lipid was conjugated at one of positions (P) 1, 4, 8, 12, 13, 18, 20, 23, 28, 29, and 30 in the sense strand as shown in the modification patterns below. The unmodified sense and antisense strands are provided in SEQ ID NOs: 756 and 757, respectively, and the modified strands are shown in Table 31.

### Tetraloop RNAi Oligonucleotide Modification Patterns:

P1 Sense Strand:
[ademXs-C16][mX][mX][mX][mX][mX][mX][mX][mX][mX][mX][mX][mX][mX][mX][mX]
[mX][mX][mX][mX][mX][mX][mX][mX][mX][mX][mX][mX][mX][mX][mX][mX][mX][mX]
[mX][mX]
P4 Sense Strand:
[mXs][mX][mX][ademX-C16][mX][mX][mX][fX][fX][fX][fX][mX][mX][mX][mX][mX]
[mX][mX][mX][mX][mX][mX][mX][mX][mX][mX][mX][mX][mX][mX][mX][mX][mX]
[mX][mX][mX]
P8 Sense Strand:
[mXs][mX][mX][mX][mX][mX][mX][ademX-C16][fX][fX][fX][mX][mX][mX][mX]
[mX][mX][mX][mX][mX][mX][mX][mX][mX][mX][mX][mX][mX][mX][mX][mX][mX]
[mX][mX][mX][mX]
P12 Sense Strand:
[mXs][mX][mX][mX][mX][mX][mX][fX][fX][fX][fX][ademX-C16][mX][mX][mX][mX]
[mX][mX][mX][mX][mX][mX][mX][mX][mX][mX][mX][mX][mX][mX][mX][mX][mX]
[mX][mX][mX]
P13 Sense Strand:
[mXs][mX][mX][mX][mX][mX][mX][fX][fX][fX][fX][mX][ademX-C16][mX][mX][mX]
[mX][mX][mX][mX][mX][mX][mX][mX][mX][mX][mX][mX][mX][mX][mX][mX][mX]
[mX][mX][mX]
P18 Sense Strand:
[mXs][mX][mX][mX][mX][mX][mX][fX][fX][fX][fX][mX][mX][mX][mX][mX][mX]
[ademX-C16][mX][mX][mX][mX][mX][mX][mX][mX][mX][mX][mX][mX][mX][mX][mX]
[mX][mX][mX]
P20 Sense Strand:
[mXs][mX][mX][mX][mX][mX][mX][fX][fX][fX][fX][mX][mX][mX][mX][mX][mX][mX]
[mX][ademX-C16][mX][mX][mX][mX][mX][mX][mX][mX][mX][mX][mX][mX][mX][mX]
[mX][mX]
P23 Sense Strand:
[mXs][mX][mX][mX][mX][mX][mX][fX][fX][fX][fX][mX][mX][mX][mX][mX][mX][mX]
[mX][mX][mX][mX][ademX-
C16][mX][mX][mX][mX][mX][mX][mX][mX][mX][mX][mX][mX][mX]
P28 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][ademA-
C16][mX][mX][mX][mX][mX][mX][mX][mX]
P29 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][mX][ademX-C16][mX][mX][mX][mX]
[mX][mX][mX]
P30 Sense Strand:
[mXs][mX][mX][mX][mX][mX][mX][fX][fX][fX][fX][mX][mX][mX][mX][mX][mX][mX]
[mX][mX][mX][mX][mX][mX][mX][mX][mX][mX][mX][ademX-C16][mX][mX][mX]
[mX][mX][mX]

Each of P1, P4, P8, P12, P13, P18, P20, P23, P28, P29, and P30 hybridized to an antisense strand having the following modification pattern:

Antisense Strand:
[MePhosphonate-4O-mXs][fXs][fXs][fX][fX][mX][fX]
[mX][mX][fX][mX][mX][mX][fX][mX][mX][mX][mX][mX]
[mXs][mXs][mX]

### Modification Key: Table 28

TABLE 31
Tetraloop RNAi oligonucleotide lipid-conjugates

| | | Antisense |
|---|---|---|
| | Sense Strand | Strand |
| Oligonucleotide | SEQ ID NO | SEQ ID NO |
| Tetraloop P1 C16 | 759 | 771 |
| Tetraloop P4 C16 | 760 | 771 |
| Tetraloop P8 C16 | 761 | 771 |
| Tetraloop P12 C16 | 762 | 771 |
| Tetraloop P13 C16 | 763 | 771 |
| Tetraloop P18 C16 | 764 | 771 |
| Tetraloop P20 C16 | 766 | 771 |
| Tetraloop P23 C16 | 767 | 771 |
| Tetraloop P28 C16 | 768 | 771 |
| Tetraloop P29 C16 | 769 | 771 |
| Tetraloop P30 C16 | 770 | 771 |

To evaluate the oligonucleotides in Table 31, C57BL/6 female mice, 6-8 weeks old, were treated with 300 μg of RNAi oligonucleotide formulated in artificial cerebrospinal fluid via intrathecal (i.t.) lumbar injection. Target expression was assessed 7 days after injection.

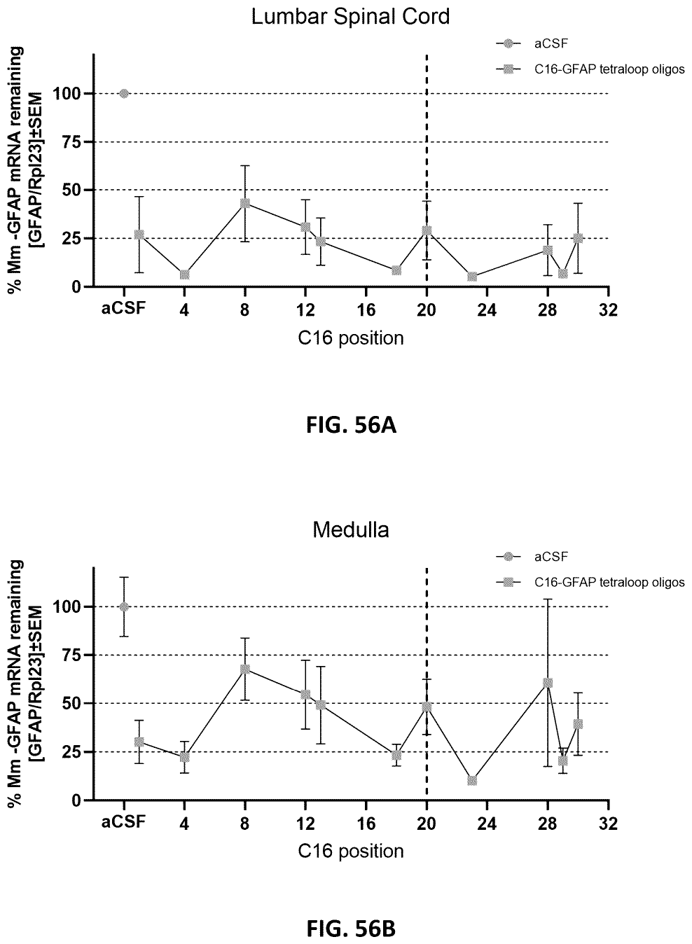

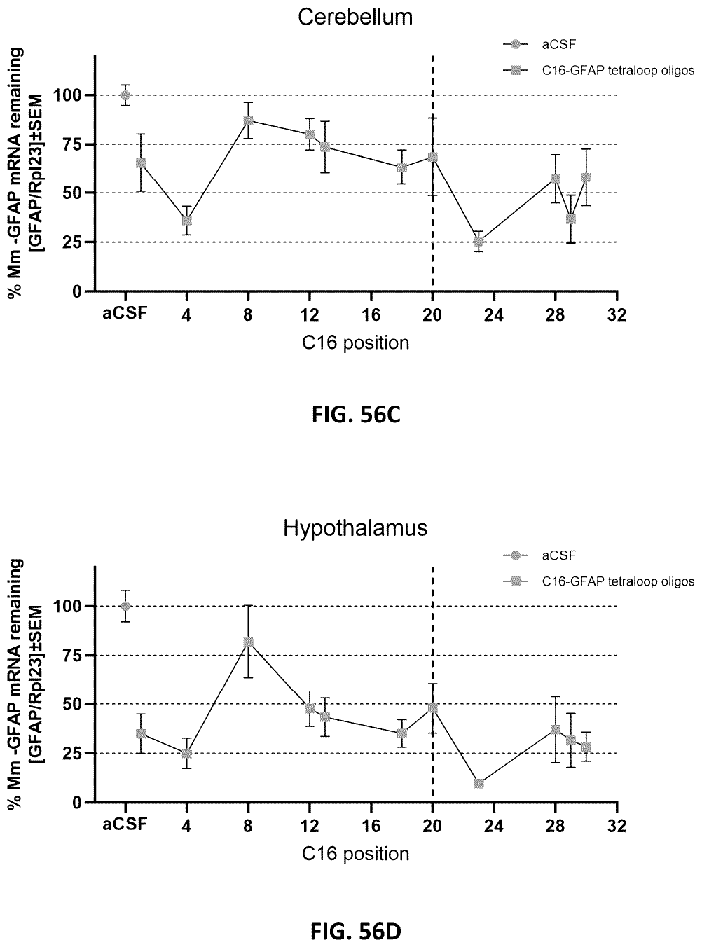

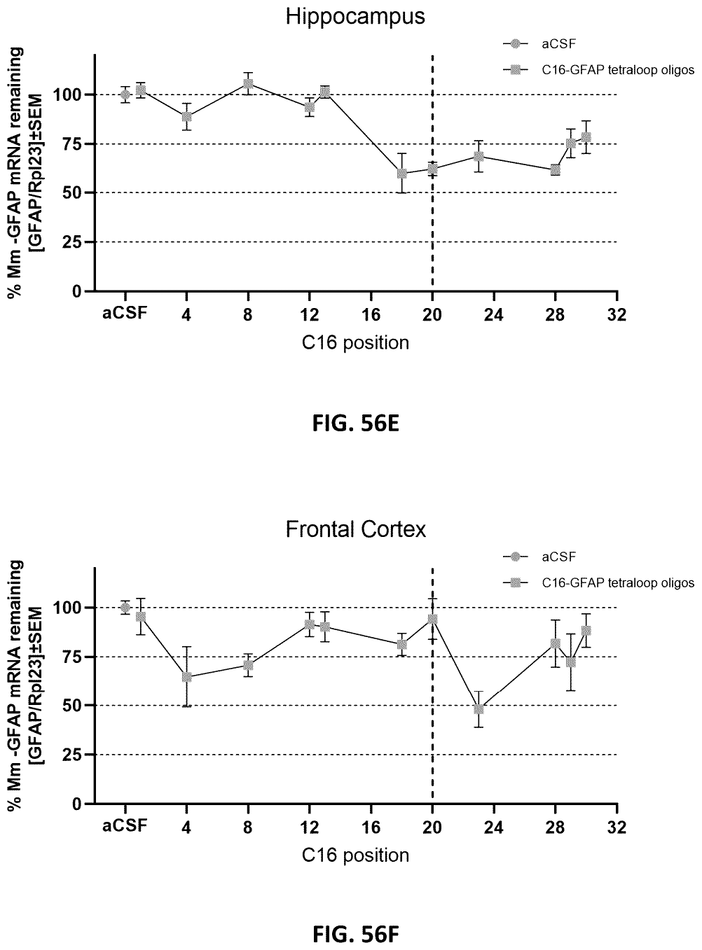

RNA was extracted from tissue samples from the lumbar spinal cord, medulla, cerebellum, hypothalamus, hippocampus, and frontal cortex to determine murine Gfap mRNA levels by qPCR as described in Example 29. Gfap mRNA expression was reduced in the lumbar spinal cord for all oligonucleotides (FIG. 56A). mRNA expression was reduced in the medulla for oligonucleotides with a C16 lipid conjugated at P1, P4, P13, P18, P20, P23, P29, or P30 (FIG. 56B). In the cerebellum, expression was reduced by oligonucleotides with a C16 lipid conjugated at P4, P23, or P29 (FIG. 56C). Gfap mRNA expression was reduced in the hypothalamus for oligonucleotides with a C16 lipid conjugated at P1, P4, P12, P13, P18, P20, P23, P28, P29, or P30 (FIG. 56D). Minimal knock-down was observed in the hippocampus and frontal cortex (FIGS. 56E and 56F, respectively). Overall, C16-conjugated tetraloop oligonucleotides reduced Gfap expression throughout several CNS tissues following administration to the CNS via i.t. lumbar injection.

### Example 33: Synthesis of Lipid-Conjugated Blunt-End Oligonucleotides

The following schematic depicts the synthesis of a blunt end oligonucleotide with a C16-lipid at the 5′-end. Lipid-conjugated blunt-ended oligonucleotides described herein can be synthesized using post-synthetic methods described in detail in PCT application No. PCT/US2021/42469. Specifically, the oligonucleotides can be synthesized using a post-synthetic conjugation approach such as that depicted below. In Eppendorf tube 1, a solution of palmitic acid in DMA was treated with HATU at rt. In Eppendorf tube 2, a solution of oligo sense strand in H₂O was treated with DIPEA. The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by reverse phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid.

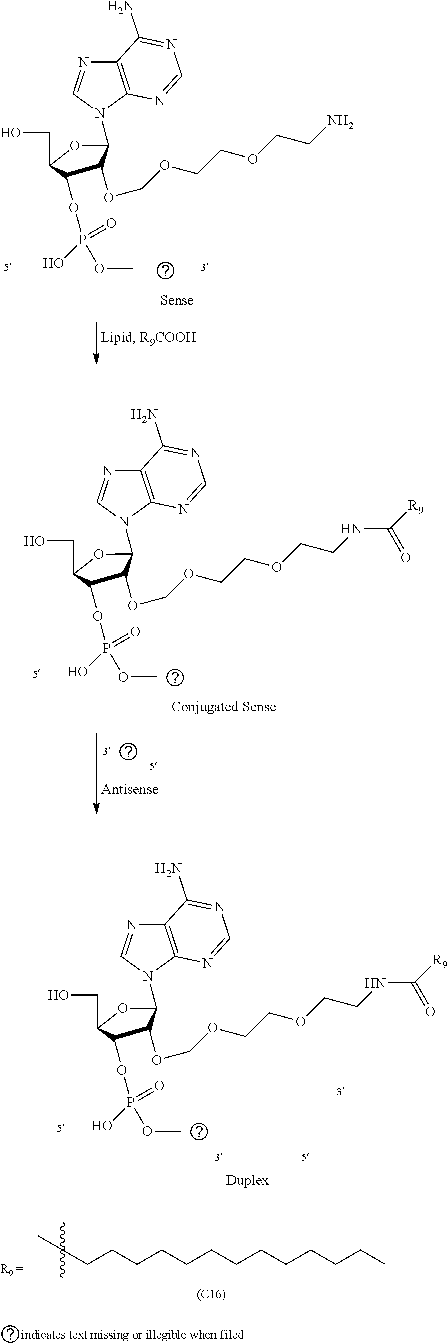

### Example 34: Positional Effects of Lipid Conjugation on the In Vivo Activity of Blunt-End RNAi Oligonucleotides to the Central Nervous System

RNAi oligonucleotides conjugated to a lipid at various positions of the sense strand were evaluated for their ability to reduce an astrocyte target expression in the CNS. RNAi oligonucleotides conjugated to a C16 lipid were generated as described above. Specifically, oligonucleotides having a blunt-end at the 3′ terminus and a 2-nucleotide overhang at the 5′ terminus were generated with a C16 lipid conjugated at positions (P) 1, 4, 8, 12, 13, 18, or 20 of the sense strand as shown in the modification patterns below. Select lipid-conjugated tetraloop oligonucleotides from Example 32 were included for comparison (P1, P4, P23, and P28). The unmodified sense and antisense strands are provided in SEQ ID NOs: 772 and 757, respectively, and the modified strands are shown in Table 32.

### Blunt-end RNAi Oligonucleotide Modification Patterns:

P1 Sense Strand:
[ademXs-C16][mX][mX][mX][mX][mX][mX][fX][fX][fX][fX][mX][mX][mX][mX][mX]
[mX][mXs][mXs][mX]
P4 Sense Strand:
[mXs][mX][mX][ademX-C16][mX][mX][mX][fX][fX][fX][fX][mX][mX][mX][mX][mX]
[mX][mXs][mXs][mX]
P8 Sense Strand:
[mXs][mX][mX][mX][mX][mX][mX][ademX-C16][fX][fX][fX][mX][mX][mX][mX][mX]
[mX][mXs][mXs][mX]
P12 Sense Strand:
[mXs][mX][mX][mX][mX][mX][mX][fX][fX][fX][fX][ademX-
C16][mX][mX][mX][mX][mX][mXs][mXs][mX]
P13 Sense Strand:
[mXs][mX][mX][mX][mX][mX][mX][fX][fX][fX][fX][mX][ademX-
C16][mX][mX][mX][mX][mXs][mXs][mX]
P18 Sense Strand:
[mXs][mX][mX][mX][mX][mX][mX][fX][fX][fX][fX][mX][mX][mX][mX][mX][mX]
[ademXs-C16][mXs][mX]
P20 Sense Strand:
[mXs][mX][mX][mX][mX][mX][mX][fX][fX][fX][fX][mX][mX][mX][mX][mX][mX]
[mXs][mXs][ademX-C16]

Each of P1, P4, P8, P12, P13, P18, and P20 hybridized to an antisense strand having the following modification pattern:

Antisense Strand:
[MePhosphonate-4O-
mXs][fXs][fXs][fX][fX][mX][fX][mX][mX][fX][mX]
[mX][mX][fX][mX][mX][mX][mX][mX][mXs][mXs][mX]

### Modification Key: Table 28

TABLE 32
Lipid-conjugated RNAi Blunt-End oligonucleotides

| | Sense Strand | Antisense Strand |
|---|---|---|
| Oligonucleotide | SEQ ID NO | SEQ ID NO |
| Blunt-End P1 C16 | 773 | 771 |
| Blunt-End P4 C16 | 774 | 771 |
| Blunt-End P8 C16 | 775 | 771 |
| Blunt-End P12 C16 | 776 | 771 |
| Blunt-End P13 C16 | 777 | 771 |
| Blunt-End P18 C16 | 778 | 771 |
| Blunt-End P20 C16 | 780 | 771 |

To evaluate the blunt-end oligonucleotide-lipid conjugates in Table 32, C57BL/6 female mice, 6-8 weeks old, were treated with 500 μg of lipid-conjugated blunt-end RNAi oligonucleotide formulated in artificial cerebrospinal fluid (aCSF) via intrathecal (i.t.) lumbar injection. Control animals were injected with aCSF only. Target expression was assessed 7 days after injection.

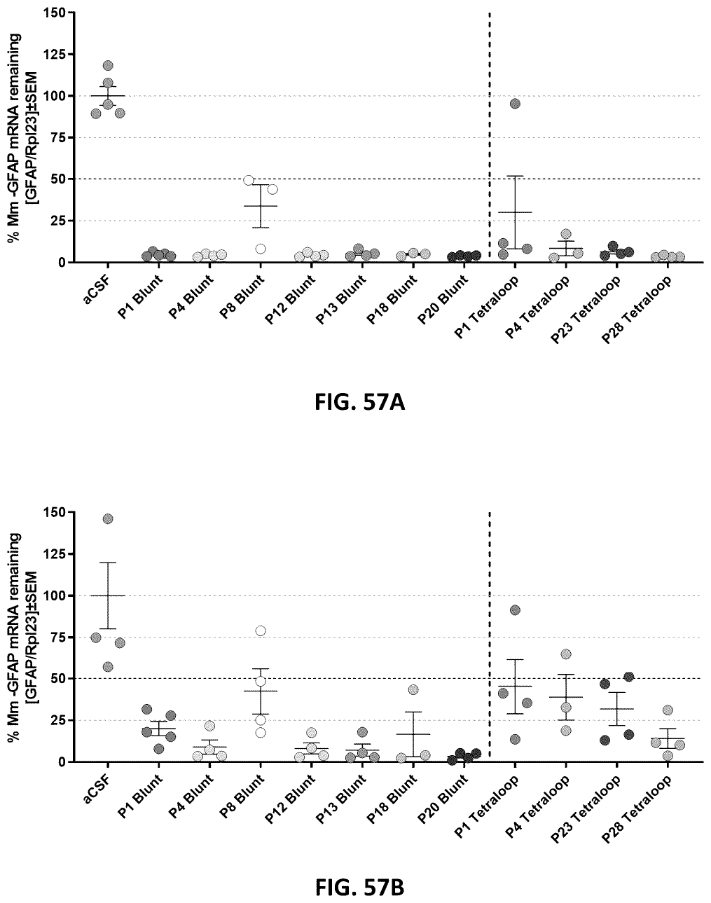

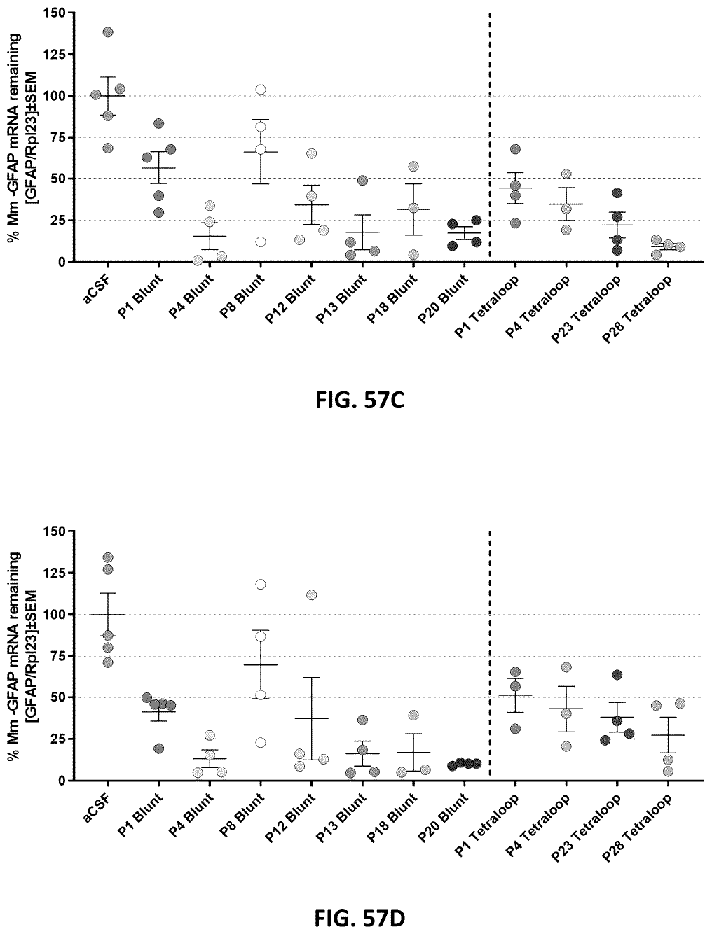

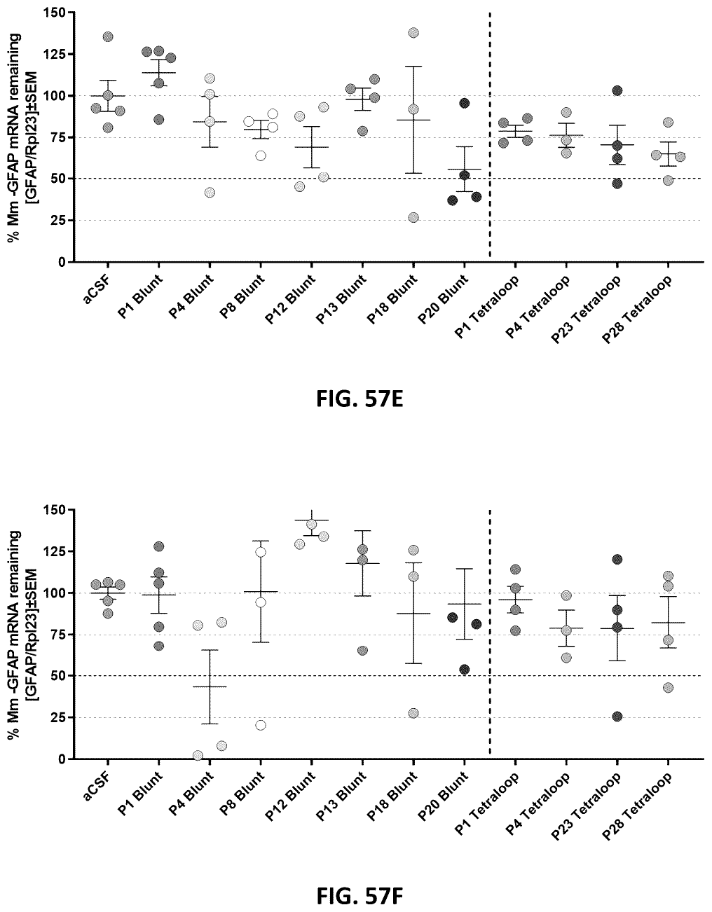

RNA was extracted from tissue samples from the lumbar spinal cord, medulla, cerebellum, hypothalamus, hippocampus, and frontal cortex to determine murine Gfap mRNA levels by qPCR (normalized to endogenous housekeeping gene Rpl23, as indicated). The levels of murine Gfap mRNA were determined using the methods described in Example 29. Gfap mRNA expression was reduced by about 50% or greater in samples from several tissues of the CNS (FIGS. 57A-57F). Specifically, all lipid conjugation positions significantly reduced Gfap mRNA in lumbar spinal cord and medulla (FIGS. 57A and 57B). mRNA expression was reduced in the cerebellum for oligonucleotides with a C16 lipid conjugated at P4, P12, P13, P18, or P20 (FIG. 57C). In the hypothalamus, expression was reduced by oligonucleotides with a C16 lipid conjugated at P1, P4, P12, P13, P18, or P20 (FIG. 57D). Minimal inhibition of Gfap mRNA was observed in the hippocampus and frontal cortex (FIGS. 57E and 57F, respectively). For each of the brain regions, the lipid-conjugated tetraloop oligonucleotides tested demonstrated similar expression reduction as that shown in Example 32. Overall, several positions of lipid-conjugation across blunt-end oligonucleotide are successful inhibitors of target mRNA in the CNS.

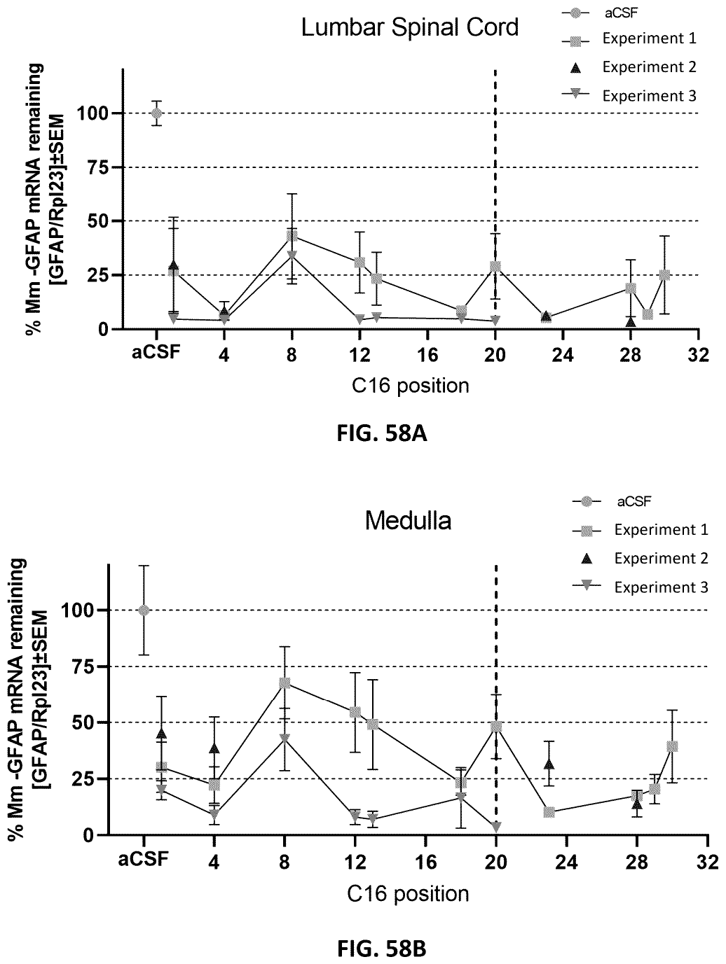

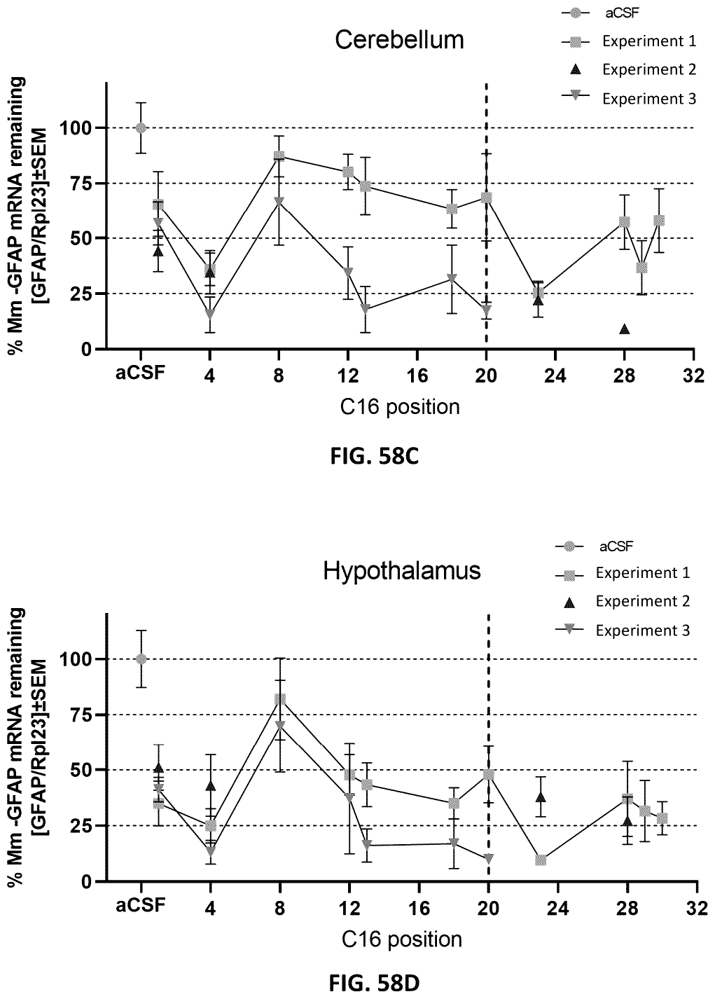

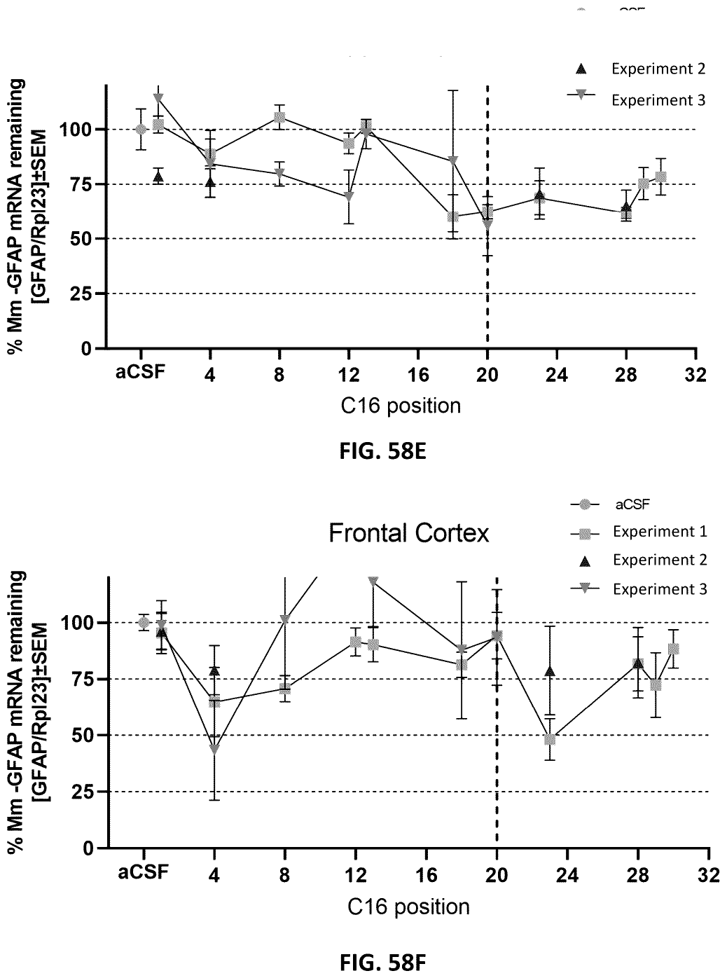

The percent remaining mRNA from the experiments described in Example 32 and the present example was compared. Specifically, the data is summarized in FIGS. 58A-58F to demonstrate potency across brain regions and lipid-positions in tetraloop and blunt-end oligonucleotides.

### Example 35: C16-Conjugated GFAP Blunt-End Oligonucleotides Inhibit Mouse Gfap In Vivo in a Concentration Dependent Manner

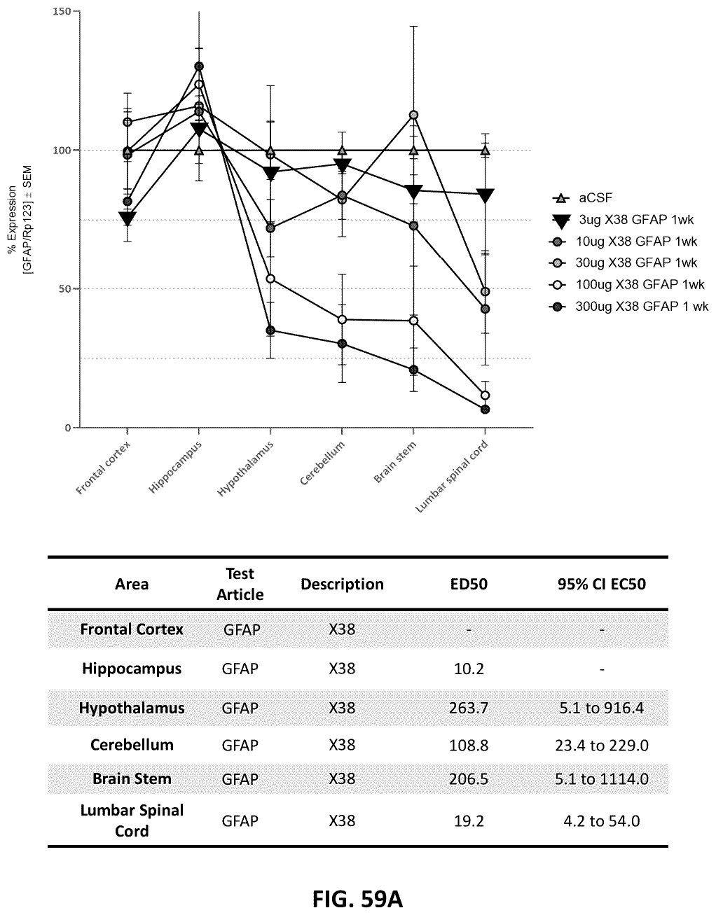

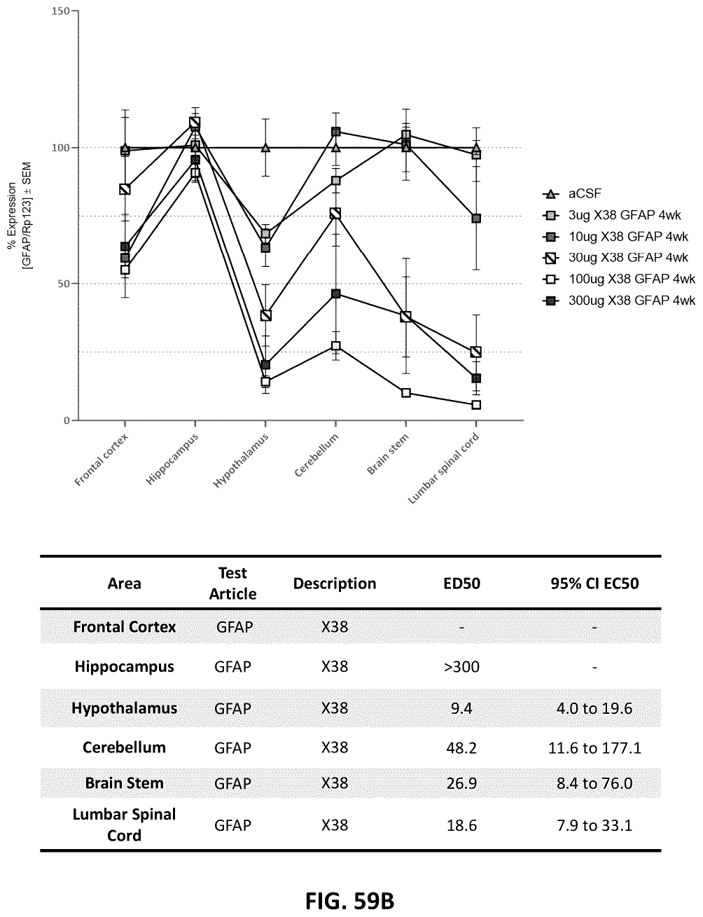

A blunt-end oligonucleotide with a C16 lipid conjugated at position 1 was assessed for the ability to reduce murine Gfap in the central nervous system (CNS) via i.t. administration in a concentration-dependent manner. Specifically, mice were administered 3, 10, 30, 100, or 300 μg of GFAP-1477 (SEQ ID NO: 773 (sense strand) and SEQ ID NO: 771 (antisense strand)) formulated in aCSF via i.t. injection. Animals were sacrificed 7- or 28-days following i.t. injection. RNA was extracted from liver tissue as described in Example 29. Reduction of Gfap expression was observed in a concentration-dependent manner across several tissues of the CNS. Specifically, at 7 days following injection, expression was reduced in the hypothalamus, cerebellum, brain stem, and lumbar spinal cord (FIG. 59A). The reduction was maintained out to 28-days in the same tissues, indicating long-term inhibition after a single administration of blunt-end oligonucleotide (FIG. 59B). Together, this data demonstrates the long-term potency of lipid-conjugated blunt-end oligonucleotides to inhibit astrocyte target mRNA.

### Example 36: Preparation of RNAi Oligonucleotides

### Oligonucleotide Synthesis and Purification

The oligonucleotides (RNAi oligonucleotides) described in the foregoing Examples were chemically synthesized using methods described herein. Generally, RNAi oligonucleotides were synthesized using solid phase oligonucleotide synthesis methods as described for 19-23mer RNAi oligonucleotides (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g. Hughes and Ellington (2017) COLD SPRING HARB PERSPECT BIOL. 9(1):a023812; Beaucage S. L., Caruthers M. H. STUDIES ON NUCLEOTIDE CHEMISTRY V: Deoxynucleoside Phosphoramidites—A New Class of Key Intermediates for Deoxypolynucleotide Synthesis, TETRAHEDRON LETT. 1981; 22:1859-62. doi: 10.1016/S0040-4039(01) 90461-7; PCT application No. PCT/US2021/42469 (each of which incorporated herein by this reference)). RNAi oligonucleotides having a 19mer core sequence were formatted into constructs having a 36mer sense strand and a 22mer antisense strand to allow for processing by the RNAi machinery. The 19mer core sequence is complementary to a region in the UGT8 mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84) and the phosphoramidite synthesis as shown below.

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

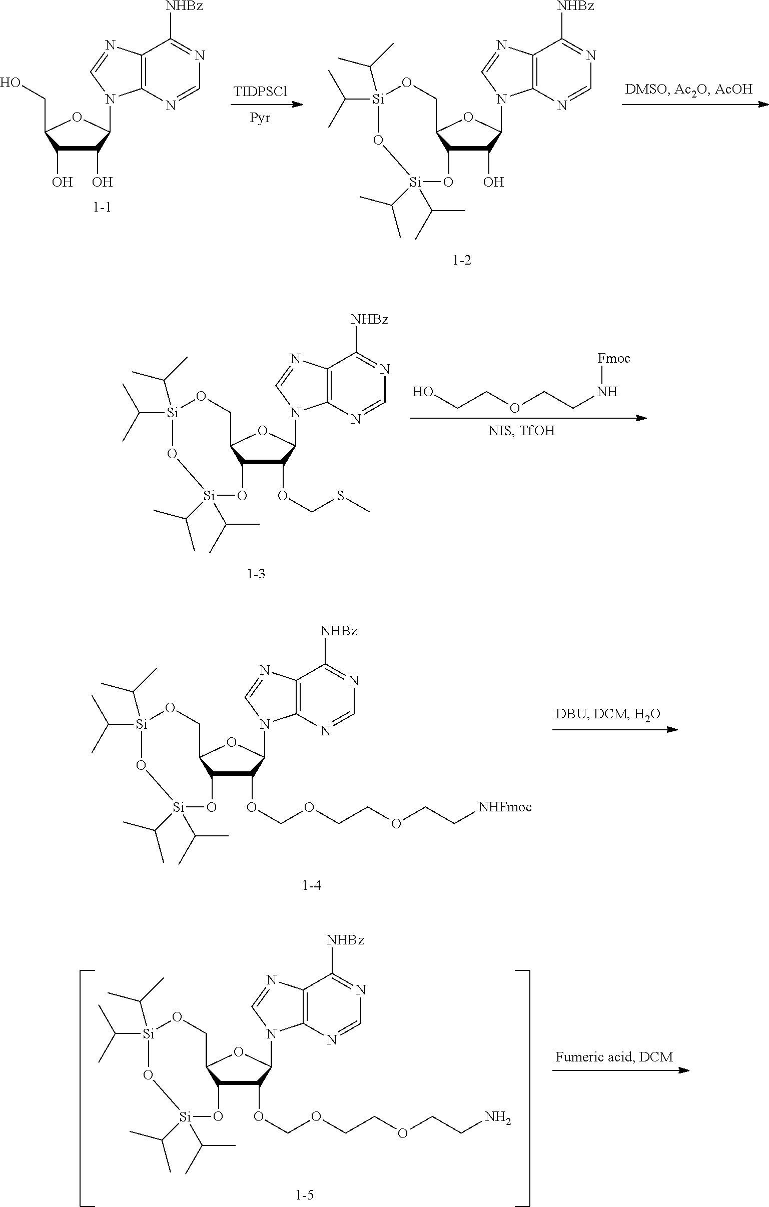

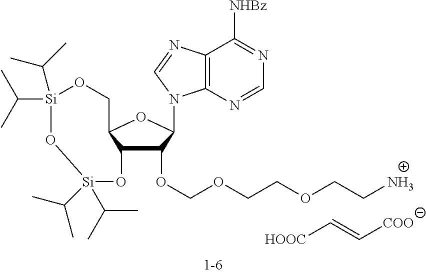

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHZ, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

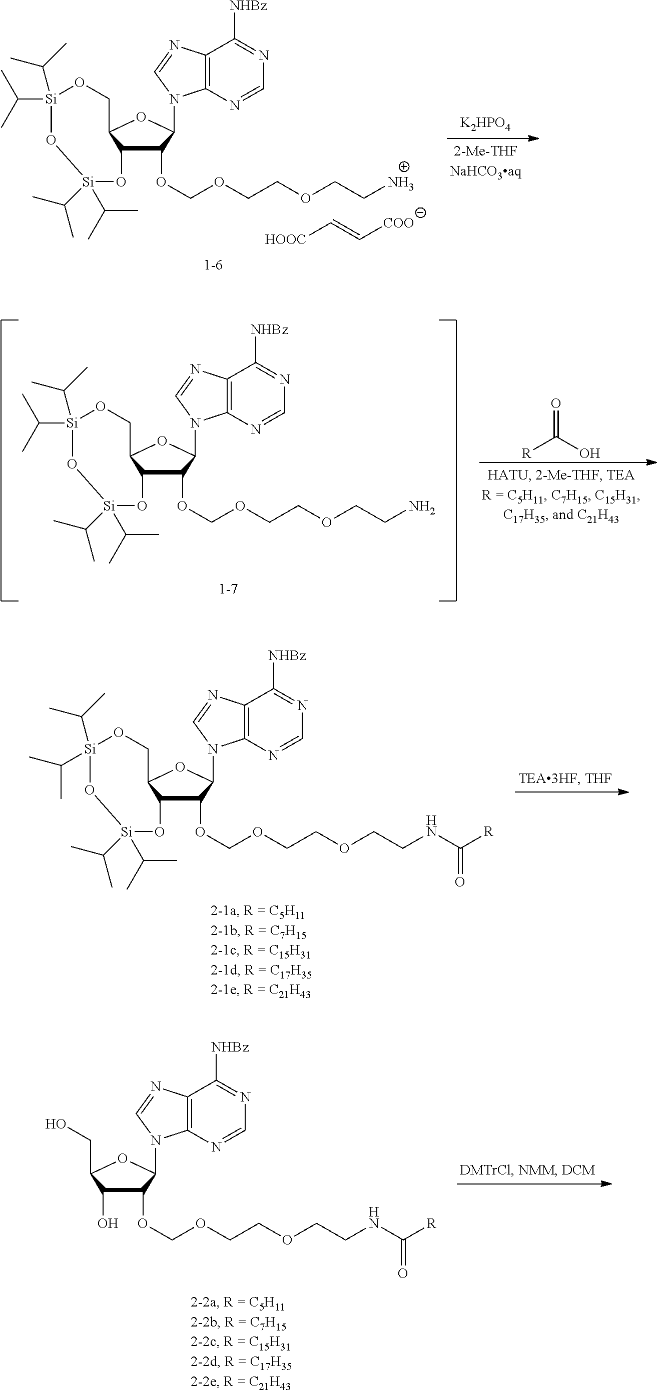

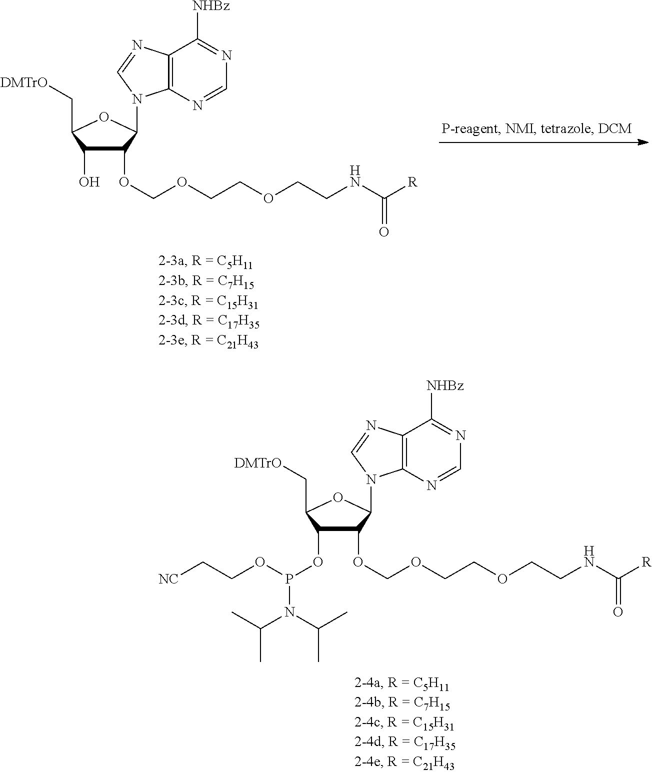

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHZ, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHZ, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHZ, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

The synthesis methods described herein are used to generate the lipid-conjugated oligonucleotides described in Example 38.

### Example 37: GalNAc-Conjugated UGT8 RNAi Oligonucleotides Inhibit Human UGT8 In Vivo

Uridine diphosphate Glycosyltransferase 8 (UGT8) is an enzyme involved in synthesizing galactosylceramide which is a glycosphingolipid of myelin. This product is primarily produced by oligodendrocytes in the central nervous system (CNS), and thus sufficient knockdown of UGT8 indicates ability of oligonucleotides described herein to reduce expression of target genes expressed in oligodendrocytes. To evaluate the ability of RNAi oligonucleotides to reduce UGT8 expression in vivo, an HDI mouse model was used. Specifically, oligonucleotides synthesized as described in Example 36 were used to generate double-stranded RNAi oligonucleotides comprising a nicked tetraloop GalNAc-conjugated structure (referred to herein as “GalNAc-conjugated UGT8 oligonucleotides” or “GalNAc-UGT8 oligonucleotides”) having a 36-mer passenger strand and a 22-mer guide strand. Further, the nucleotide sequences comprising the passenger strand and guide strand have a distinct pattern of modified nucleotides and phosphorothioate linkages. Three of the nucleotides comprising the tetraloop were each conjugated to a GalNAc moiety (CAS #14131-60-3). The modification pattern of each strand is illustrated below:

Sense Strand:
5′-[mXs][mX][mX][mX][mX][mX][mX][fX][fX][fX][fX][mX][mX][mX][mX]
[mX][mX][mX][mX][mX][mX][mX][mX][mX][mX][mX][mX][ademX-GalNAc][ademX-
GalNAc][ademX-GalNAc][mX][mX][mX][mX][mX][X]-3′

### Hybridized to:

Antisense Strand:
5′[MePhosphonate-4O-mXs][fXs][fX][fX][fX][mX]
[mX][mX][mX][fX][mX][mX][mX][fX][mX][mX][mX][mX]
[mX][mXs][mXs][mX]3′

TABLE 33
Modification key:

| Symbol | Modification/linkage |
|---|---|
| [MePhosphonate- | 4′-O-monomethylphosphonate-2′- |
| 4O-mX] | O-methyl modified nucleotide |
| [ademX-GalNAc] | GalNAc attached to a nucleotide |
| [mXs] | 2′-O-methyl modified nucleotide with a |
| | phosphorothioate linkage to the |
| | neighboring nucleotide |
| [fXs] | 2′-fluoro modified nucleotide with a |
| | phosphorothioate linkage to the |
| | neighboring nucleotide |
| [mX] | 2′-O-methyl modified nucleotide with |
| | phosphodiester linkages to |
| | neighboring nucleotides |
| [fX] | 2′-fluoro modified nucleotide with |
| | phosphodiester linkages to |
| | neighboring nucleotides |
| [ademX-C16] | C16 lipid attached to a nucleotide |
| [ademAs-C16] | C16 lipid attached to adenine nucleotide |
| | with a phosphorothioate linkage to the |
| | neighboring nucleotide |
| [ademCs-C16] | C16 lipid attached to cystosine nucleotide |
| | with a phosphorothioate linkage |
| | to the neighboring nucleotide |
| [ademXs-C16] | C16 lipid attached to a nucleotide |
| | with a phosphorothioate linkage to the |
| | neighboring nucleotide |
| [+X] | Locked Nucleic Acid |

Nine GalNAc-conjugated UGT8 oligonucleotides shown in Table 34 were evaluated. The oligonucleotides selected were based on a previous screen to identify potent UGT8-targeting oligonucleotides (data not shown). Oligonucleotides were evaluated in mice engineered to transiently express human UGT8 mRNA in hepatocytes of the mouse liver. Briefly, 6-week-old female CD-1 mice (n=5) were subcutaneously administered the indicated GalNAc-conjugated UGT8 oligonucleotides at a concentration of 0.3 mg/kg or 1 mg/kg formulated in PBS. A control group of mice (n=5) were administered only PBS. Three days later (72 hours), the mice were hydrodynamically injected (HDI) with 25 μg of DNA plasmid encoding the full human UGT8 gene (NM_001128174) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine UGT8 mRNA levels.

Specifically, RNA was extracted from liver tissue to determine murine UGT8 mRNA levels by qPCR (normalized to endogenous housekeeping gene Rpl23). The levels of human UGT8 mRNA were determined using PrimeTime™ qPCR Probe Assays (IDT), which consisted of a primer pair and fluorescently labeled 5′ nuclease probe specific to murine Ugt8 mRNA. The percentage of human UGT8 mRNA remaining in the samples from treated mice was determined using the 2^{-ΔΔCt} (“delta-delta Ct”) method (Livak and Schmittgen (2001) METHODS 25:402-408). The values were normalized for transfection efficiency using the NeoR gene included on the DNA plasmid.

TABLE 34
GalNAc-Conjugated Human UGT8 RNAi
Oligonucleotides for HDI screen

| | | Un- | Un- | | |
|---|---|---|---|---|---|
| | | modified | modified | Modified | Modified |
| | Species | Sense | Antisense | Sense | Antisense |
| | Targets | Strand | strand | Strand | strand |
| UGT8-277 | Hs/Mf/Mm | 804 | 813 | 822 | 831 |
| UGT8-278 | Hs/Mf/Mm | 805 | 814 | 823 | 832 |
| UGT8-505 | Hs/Mf/Mm | 806 | 815 | 824 | 833 |
| UGT8-508 | Hs/Mf/Mm | 807 | 816 | 825 | 834 |
| UGT8-509 | Hs/Mf/Mm | 808 | 817 | 826 | 835 |
| UGT8-513 | Hs/Mf/Mm | 809 | 818 | 827 | 836 |
| UGT8-616 | Hs/Mf/Mm | 810 | 819 | 828 | 837 |
| UGT8-843 | Hs/Mf/Mm | 811 | 820 | 829 | 838 |
| UGT8-1726 | Hs/Mf/Mm | 812 | 821 | 830 | 839 |

*Hs/Mf/Mm represents that the oligonucleotide recognizes human, monkey, and mouse UGT8

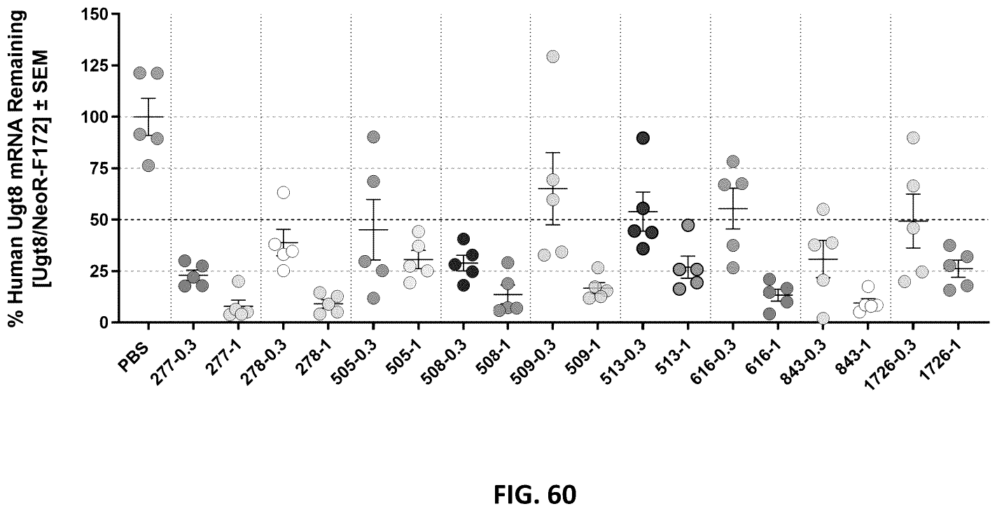

As shown in FIG. 60, each oligonucleotide reduced UGT8 expression by at least 50% at 1 mg/kg. UGT8-277 was the most potent oligonucleotide and reduced UGT8 expression by more than 75% at the lower 0.3 mg/kg concentration. This data demonstrates that GalNAc-modified oligonucleotides successfully reduce UGT8 expression in vivo.

### Example 38: RNAi Oligonucleotide Inhibition of UGT8 Expression in Mice Via Intrathecal Injection

The UGT8-277 oligonucleotide described in Example 37 was assessed for the ability to reduce murine Ugt8 expression in the central nervous system (CNS) following intrathecal (i.t.) administration. Specifically, mice were administered a single bolus of 10, 32, 100, 320, or 500 μg of UGT8-277, with a tetraloop having 2′-O-methyl (2′OMe) modified nucleotides (SEQ ID NO: 841 (sense strand) and SEQ ID NO: 831 (antisense strand)), formulated in artificial cerebrospinal fluid (aCSF) via i.t. lumbar injections. The modification patterns of each strand are illustrated below:

No Ligand Sense Strand:
5′-[mXs][mX][mX][mX][mX][mX][mX][fX][fX][fX][fX]
[mX][mX][mX][mX][mX][mX][mX][mX][mX][mX][mX][mX]
[mX][mX][mX][mX][mX][mX][mX][mX][mX][mX][mX][mX]
[mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

### (Modification Key: Table 33)

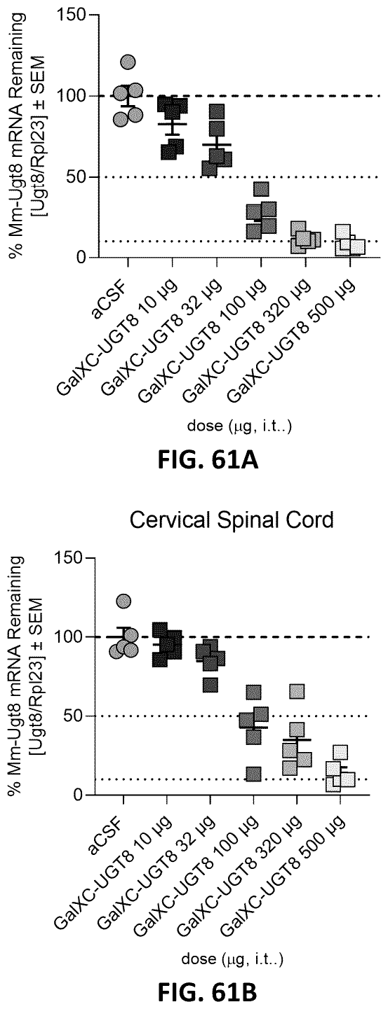

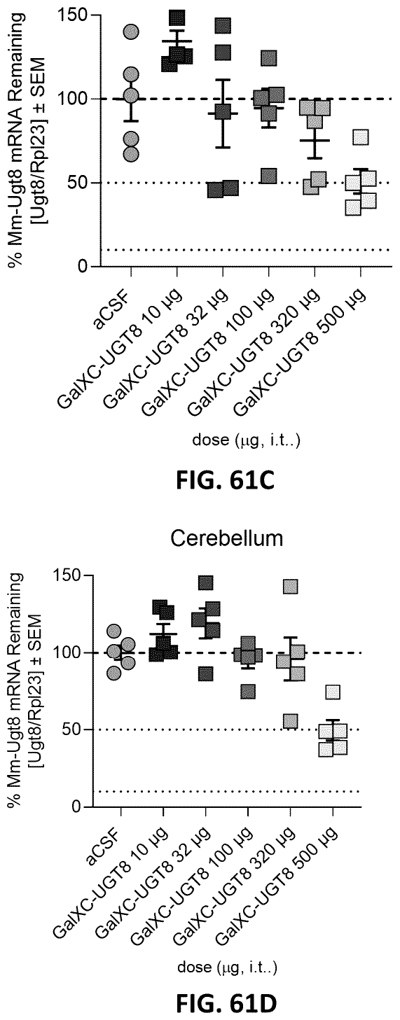

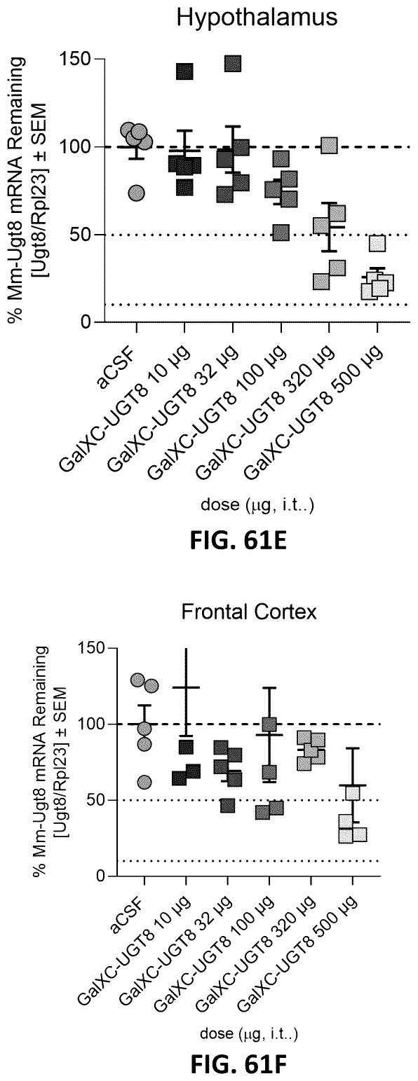

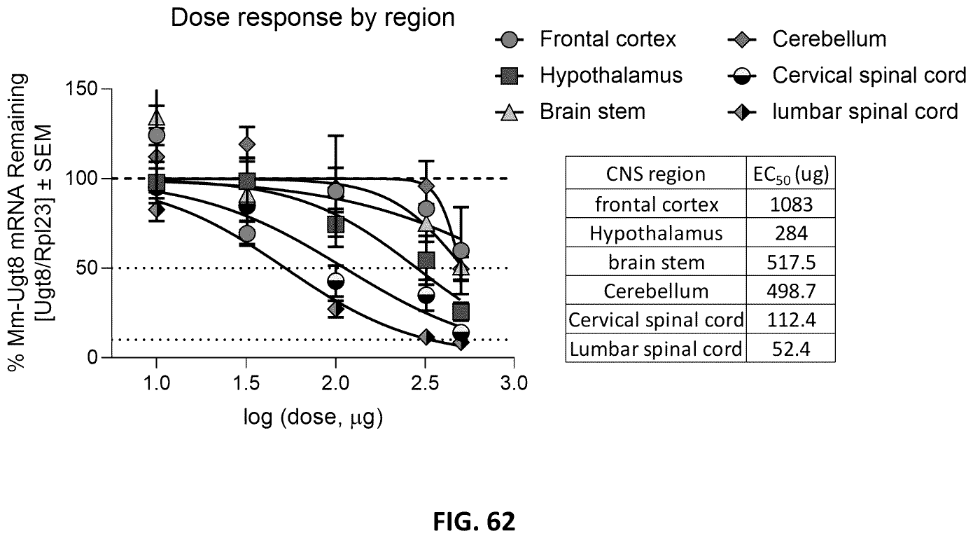

Animals were sacrificed 7 days following injection. RNA was isolated and measured as described in Example 37. Reduction of Ugt8 expression was observed in a concentration dependent manner across tissues of the CNS. Specifically, expression was reduced in lumbar spinal cord, cervical spinal cord, brainstem, cerebellum, hypothalamus, and frontal cortex as shown in FIGS. 61A-61F, respectively. EC₅₀ values were determined for each region of the brain based on the percent mRNA remaining in the tissue at each concentration of oligonucleotide (FIG. 62). Together, this data demonstrates Ugt8 targeting oligonucleotides having a 2′OMe tetraloop inhibit Ugt8 expression in several tissues of the CNS.

### Example 39: Synthesis of Tetraloop RNAi Oligonucleotide-Lipid Conjugates

Lipid-conjugated tetraloop oligonucleotides described herein can be synthesized using post-synthetic methods described in detail in PCT application No. PCT/US2021/42469. Specifically, the oligonucleotides can be synthesized using a post-synthetic conjugation approach such as that depicted below.

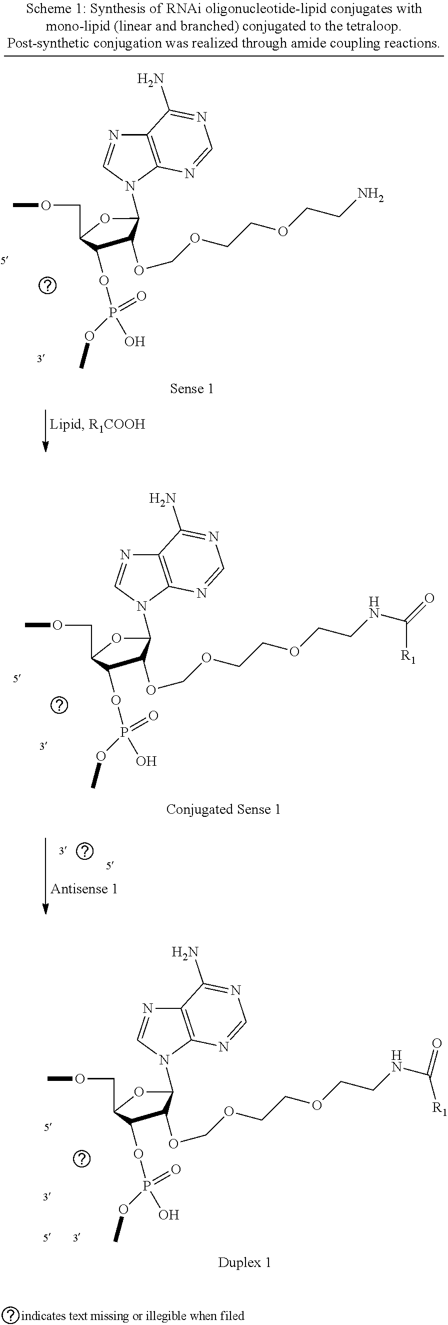

R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1. Exemplary R¹ structures are provided below:

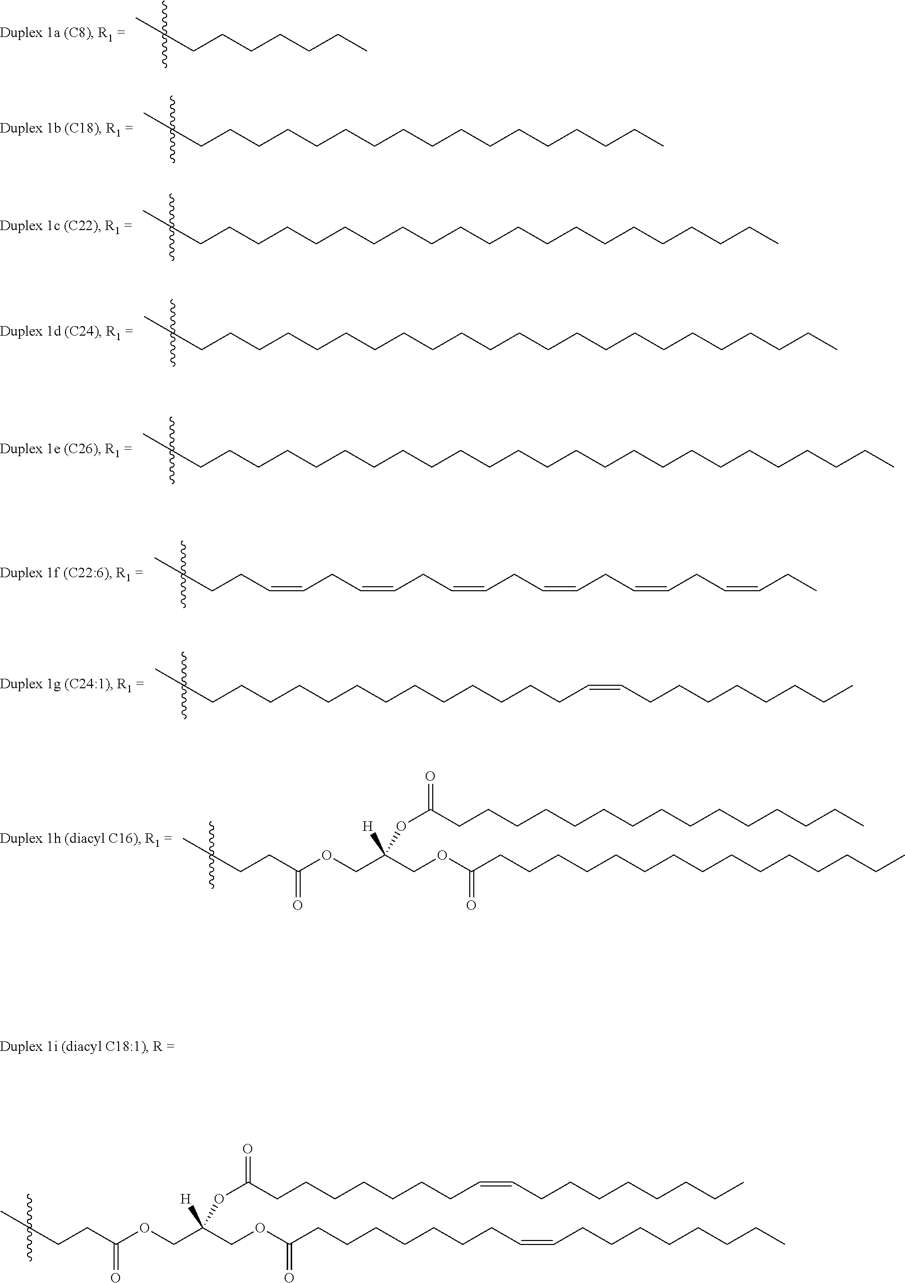

Synthesis Sense 1 and Antisense 1 were prepared by solid-phase synthesis.

Synthesis of Conjugated Sense 1a-1i.

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

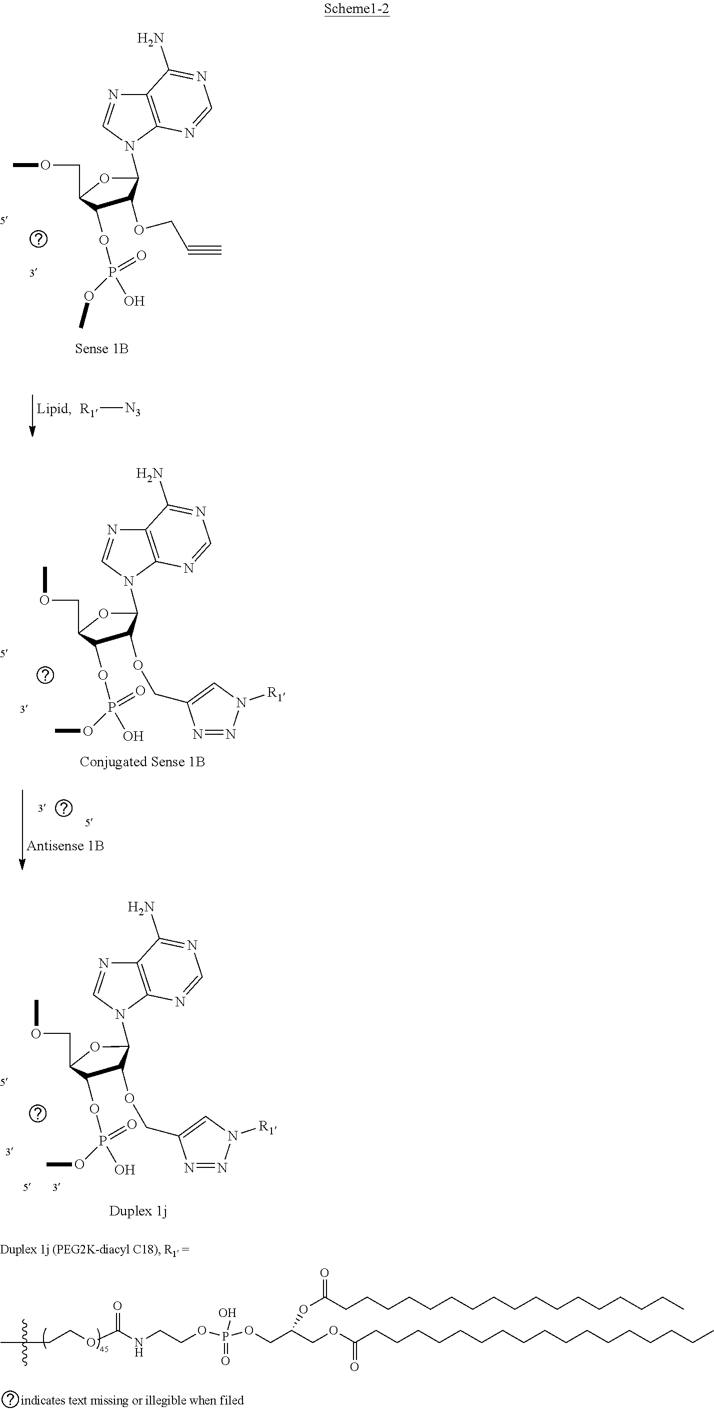

Sense 1B and Antisense 1B were prepared by solid-phase synthesis.

### Synthesis of Conjugated Sense 1j.

In Eppendorf tube 1, a solution of oligo (10.00 mg, 0.8 μmol) in a 3:1 mixture of DMA/H₂O (0.5 mL) was treated with the lipid linker azide (11.26 mg, 4 μmol). In Eppendorf tube 2, CuBr dimethyl sulfide (1.64 mg, 8 μmol) was dissolved in ACN (0.5 mL). Both solutions were degassed for 10 min by bubbling N₂ through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase XBridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

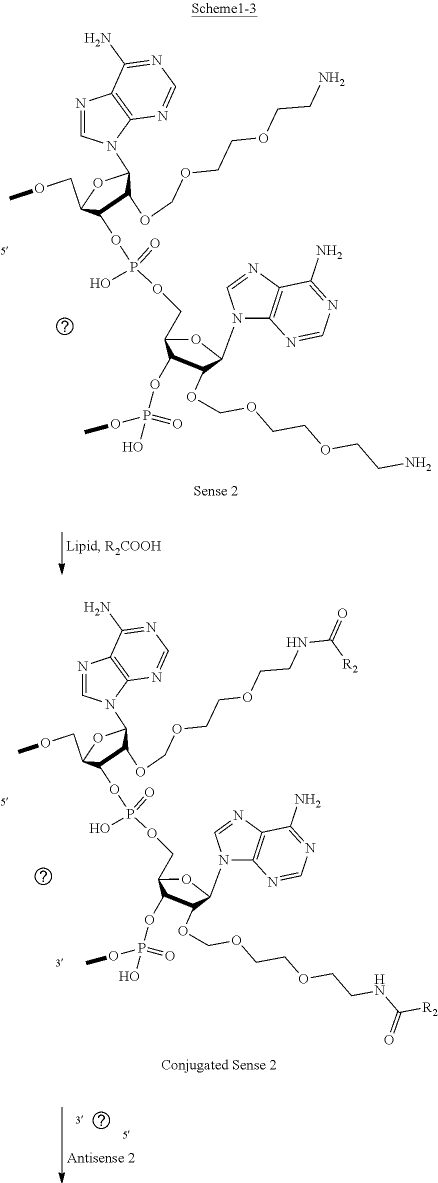

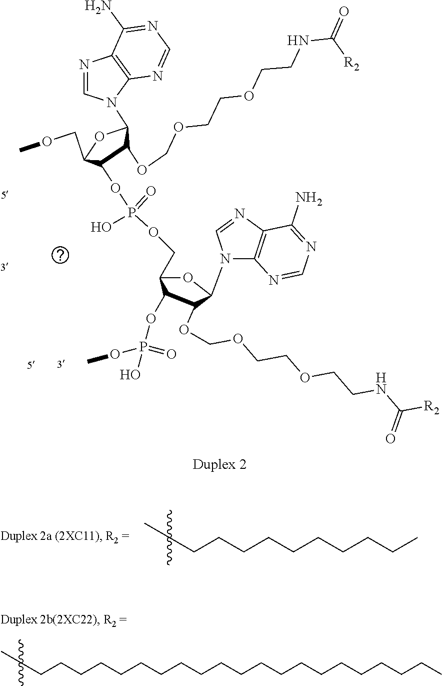

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

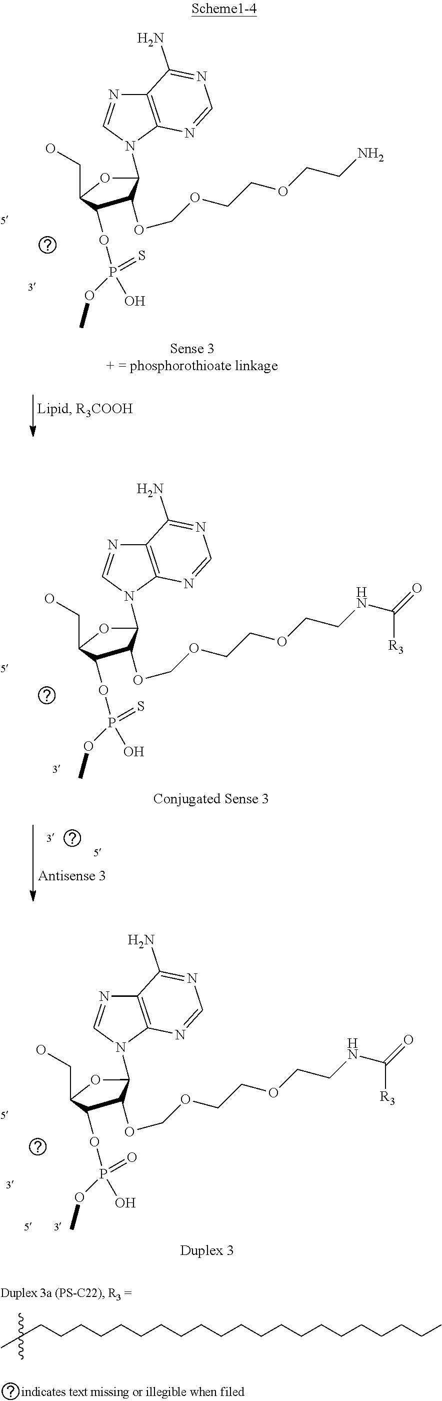

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS—C22) was prepared using the same procedures as described for the annealing of Duplex 1a (C8).

The following Scheme 1-5 depicts the synthesis of GalXC of short sense conjugated with mono-lipid using post-synthetic conjugation approach.

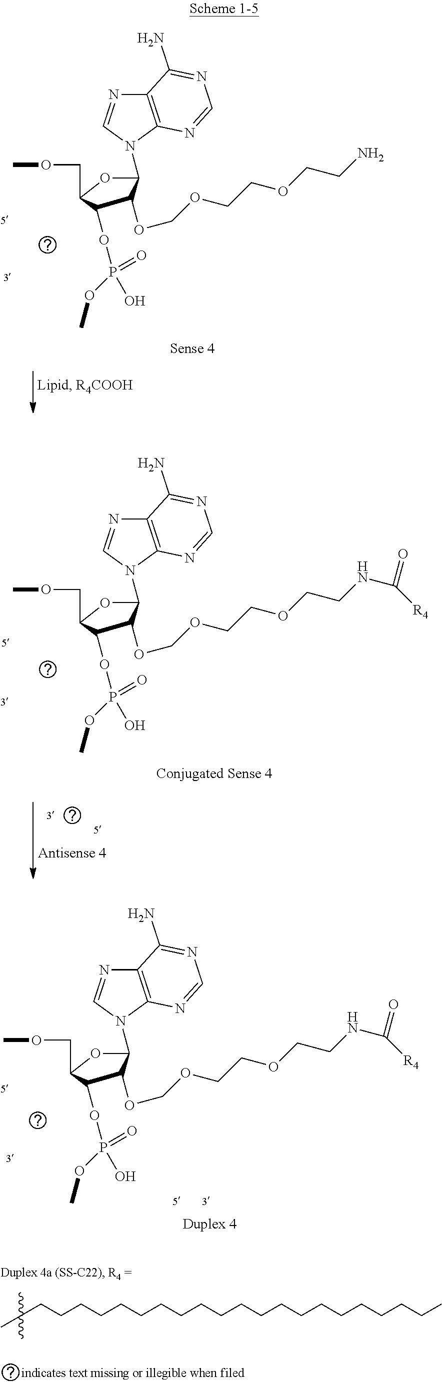

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS—C22) was prepared using the same procedures as described for the annealing of Duplex 1a (C8).

The following scheme 1-6 depicts an example of solid phase synthesis of Nicked tetraloop GalXC conjugated with lipid(s) on the loop.

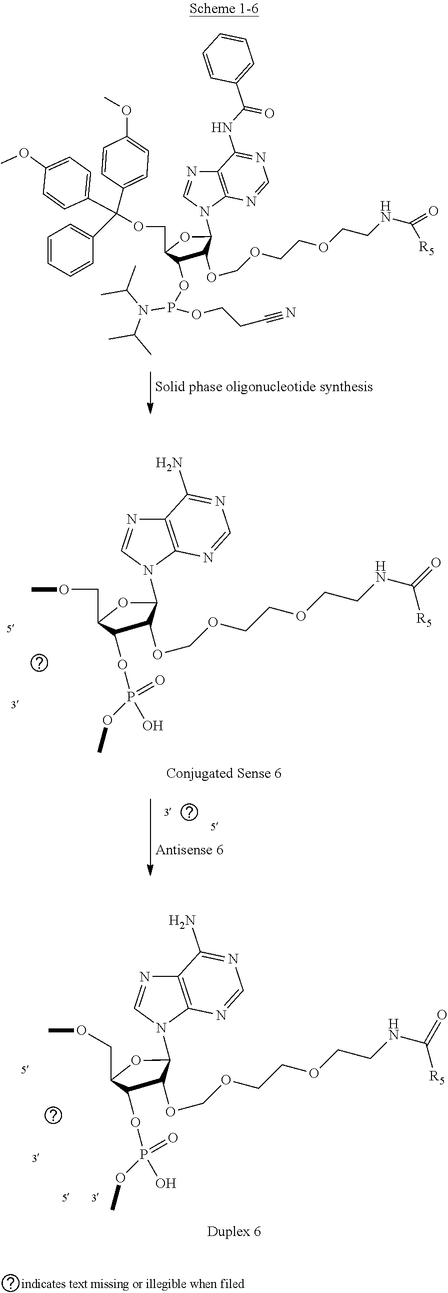

### Synthesis of Conjugated Sense 6.

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′ direction using a standard oligonucleotide synthesis protocol. 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed, and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

The following Scheme 1-7 depicts the synthesis of GalXC conjugated with mono-lipid at 5′-end using post-synthetic conjugation approach.

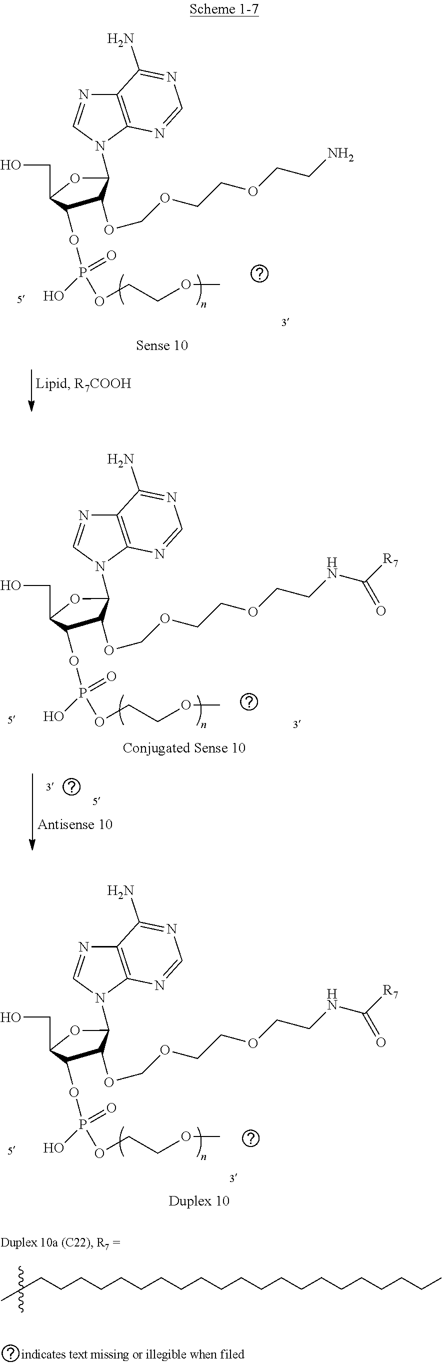

### Synthesis of Conjugated Sense 10a

Conjugated Sense 10a was obtained using the same method or a substantially similar method to the synthesis of Conjugated Sense 5.

### Synthesis Example of Duplex 10a

Duplex 10a was obtained using the same method or a substantially similar method to the synthesis of Duplex 5.

### Example 40: Positional Effects of Lipid Conjugation on the In Vivo Activity of Tetraloop RNAi Oligonucleotide Lipid-Conjugates in the Central Nervous System

The ability of RNAi oligonucleotide-lipid conjugates comprising a tetraloop to reduce mRNA expression in oligodendrocytes of the central nervous system (CNS) was evaluated in vivo. C16 conjugated Ugt8 oligonucleotides were generated by methods described in Example 36. Specifically, oligonucleotides having a tetraloop at the 3′ terminus and a 2-nucleotide overhang at the 5′ terminus were generated with a C16 lipid conjugated at a different position (positions (P) 1, 2, 3, 5, 13, 14, 15, 19, 20, 23, 28, 29, and 30) in the sense strand as shown in the patterns below. Each oligonucleotide tested comprised an antisense strand having a region of complementarity to mRNA encoding UGT8. The unmodified sense and antisense strands are provided in SEQ ID NOs: 804 and 813, respectively (UGT8-277). The modified strands are shown in Table 35.

### Tetraloop RNAi Oligonucleotide Modification Patterns:

P2 Sense Strand:
5′-[mXs][ademX-C16]
[mX][mX][mX][mX][mX][fX][fX][fX][fX][mX][mX][mX][mX][mX][mX][mX][mX][mX]
[mX][mX][mX][mX][mX][mX][mX][mX][mX][mX][mX][mX][mX][mX][mX][mX]′3′
P3 Sense Strand:
5′[mXs][mX][ademX-C16][mX][mX][mX][mX][fX][fX][fX][fX]
[mX][mX][mX][mX][mX][mX][mX][mX][mX][mX][mX][mX][mX][mX][mX]
[mX][mX][mX][mX][mX][mX][mX][mX][mX][mX]-3′
P6 Sense Strand:
5′-[mXs][mX][mX][mX][mX][ademX-C16][mX][fX][fX][fX][fX]
[mX][mX][mX][mX][mX][mX][mX][mX][mX][mX][mX][mX][mX][mX][mX][mX]
[mX][mX][mX][mX][mX][mX][mX][mX][mX]-3′
P13 Sense Strand:
5′-[mXs][mX][mX][mX][mX][mX][mX][fX][fX][fX][fX][mX][ademX-C16]
[mX][mX][mX][mX][mX][mX][mX][mX][mX][mX][mX][mX][mX][mX][mX][mX][mX]
[mX][mX][mX][mX][mX][mX]-3′
P14 Sense Strand:
5′-[mXs][mX][mX][mX][mX][mX][mX][fX][fX][fX][fX][mX][mX][ademX-C16]
[mX][mX][mX][mX][mX][mX][mX][mX][mX][mX][mX][mX][mX][mX][mX]
[mX][mX][mX][mX][mX][mX][mX]-3′
P15 Sense Strand:
5′-[mXs][mX][mX][mX][mX][mX][mX][fX][fX][fX][fX][mX][mX][mX][ademX-
C16][mX][mX][mX][mX][mX][mX][mX][mX][mX][mX][mX][mX][mX][mX][mX][mX]
[mX][mX][mX][mX][mX]-5′
P19 Sense Strand:
5′-[mXs][mX][mX][mX][mX][mX][mX][fX][fX][fX][fX][mX][mX][mX][mX][mX]
[mX][mX][ademX-C16][mX][mX][mX][mX][mX][mX][mX][mX][mX][mX]
[mX][mX][mX][mX][mX][mX][mX]-3′
P20 Sense Strand:
5′-[mXs][mX][mX][mX][mX][mX][mX][fX][fX][fX][fX][mX][mX][mX][mX][mX]
[mX][mX][mX][ademX-C16][mX][mX][mX][mX][mX][mX][mX]
[mX][mX][mX][mX][mX][mX][mX][mX][mX]-3′
P23 Sense Strand:
5′-[mXs][mX][mX][mX][mX][mX][mX][fX][fX][fX][fX][mX][mX][mX][mX][mX][mX]
[mX][mX][mX][mX][mX][ademX-C16][mX][mX][mX][mX][mX][mX][mX][mX]
[mX][mX][mX][mX][mX]-3′
P28 Sense Strand:
5′[mXs][mX][mX][mX][mX][mX][mX][fX][fX][fX][fX][mX][mX][mX][mX][mX][mX]
[mX][mX][mX][mX][mX][mX][mX][mX][mX][mX][ademX-C16][mX][mX][mX][mX]
[mX][mX][mX][mX]-3′
P29 Sense Strand:
5′-[mXs][mX][mX][mX][mX][mX][mX][fX][fX][fX][fX][mX][mX][mX][mX][mX][mX]
[mX][mX][mX][mX][mX][mX][mX][mX][mX][mX][mX][ademX-C16][mX]
[mX][mX][mX][mX][mX][mX]-3′
P30 Sense Strand:
5′-[mXs][mX][mX][mX][mX][mX][mX][fX][fX][fX][fX][mX][mX][mX][mX][mX]
[mX][mX][mX][mX][mX][mX][mX][mX][mX][mX][mX][mX][mX][ademX-
C16][mX][mX][mX][mX][mX][mX]-3′
P1 AdemC C16 Sense Strand:
5′-[ademCs-C16][mX][mX][mX][mX][mX][mX][fX][fX][fX]
[fX][mX][mX][mX][mX][mX][mX][mX][mX][mX][mX][mX][mX][mX][mX][mX][mX]
[mX][mX][mX][mX][mX][mX][mX][mX][mX]-3′
P1 AdemA C16 Mismatch Sense Strand:
5′-[ademAs-C16][mX][mX][mX][mX][mX][mX][fX][fX][fX][fX][mX]
[mX][mX][mX][mX][mX][mX][mX][mX][mX][mX][mX][mX][mX][mX][mX][mX]
[mX][mX][mX][mX][mX][mX][mX][mX]-3′

Each of P1, P2, P3, P6, P13, P14, P15, P19, P20, P23, P28, P29, and P30 hybridized to an antisense strand having the following modification pattern:

Antisense Strand:
[MePhosphonate-4O-mXs][fXs][fXs][fX][fX][mX][fX]
[mX][mX][fX][mX][mX][mX][fX][mX][mX][mX][mX][mX]
[mXs][mXs][mX]

### Modification Key: Table 33

TABLE 35
Lipid-conjugated RNAi Tetraloop Oligonucleotides

| | | Antisense |
|---|---|---|
| | Sense Strand | Strand |
| Oligonucleotide | SEQ ID NO | SEQ ID NO |
| Tetraloop No Ligand | 841 | 840 |
| Tetraloop P2 C16 | 842 | 840 |
| Tetraloop P3 C16 | 843 | 840 |
| Tetraloop P6 C16 | 844 | 840 |
| Tetraloop P13 C16 | 845 | 840 |
| Tetraloop P14 C16 | 846 | 840 |
| Tetraloop P15 C16 | 847 | 840 |
| Tetraloop P19 C16 | 848 | 840 |
| Tetraloop P20 C16 | 849 | 840 |
| Tetraloop P23 C16 | 850 | 840 |
| Tetraloop P28 C16 | 851 | 840 |
| Tetraloop P29 C16 | 852 | 840 |
| Tetraloop P30 C16 | 853 | 840 |
| Tetraloop P1 AdemC C16* | 854 | 840 |
| Tetraloop P1 AdemA C16 mismatch* | 855 | 840 |

*Position 1 of UGT8-277 sense strand (SEQ ID NO: 804) is a cytosine. For ademC C16 the cytosine was conjugated with a C16 lipid. For the ademA C16 mismatch the first nucleotide was modified to an adenine and conjugated with a C16 lipid.

To evaluate the tetraloop RNAi oligonucleotide lipid-conjugates in Table 35, C57BL/6 female mice, 6-8 weeks old, were treated with 300 μg of lipid-conjugated tetraloop RNAi oligonucleotide formulated in artificial cerebrospinal fluid (aCSF) via (i.t.) lumbar injection. Control animals were injected with aCSF only. Target knockdown was assessed 7 days after injection.

RNA was extracted from tissue samples from the lumbar spinal cord, medulla, hippocampus, and frontal cortex to determine murine Ugt8 mRNA levels by qPCR (normalized to endogenous housekeeping gene Rpl23, as indicated) as described in Example 37.

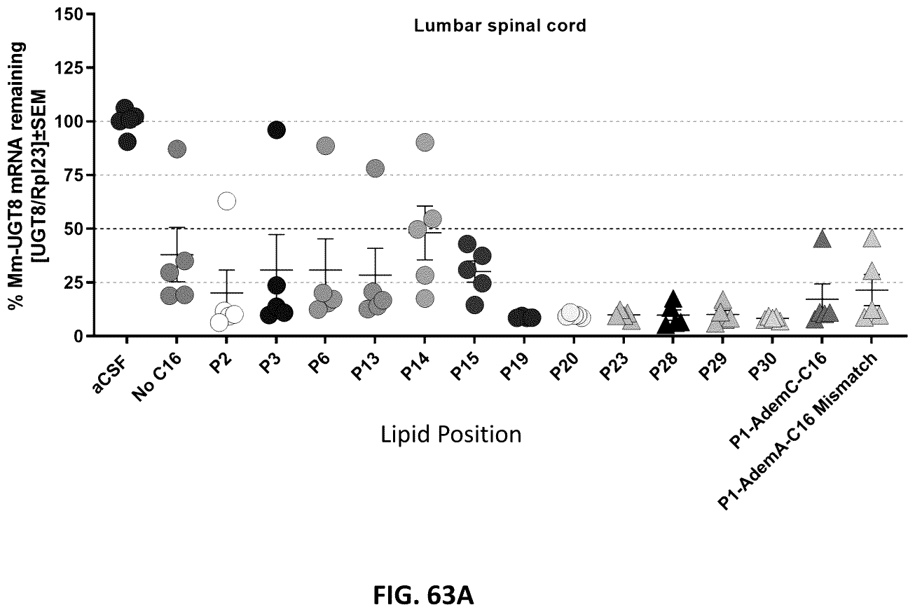

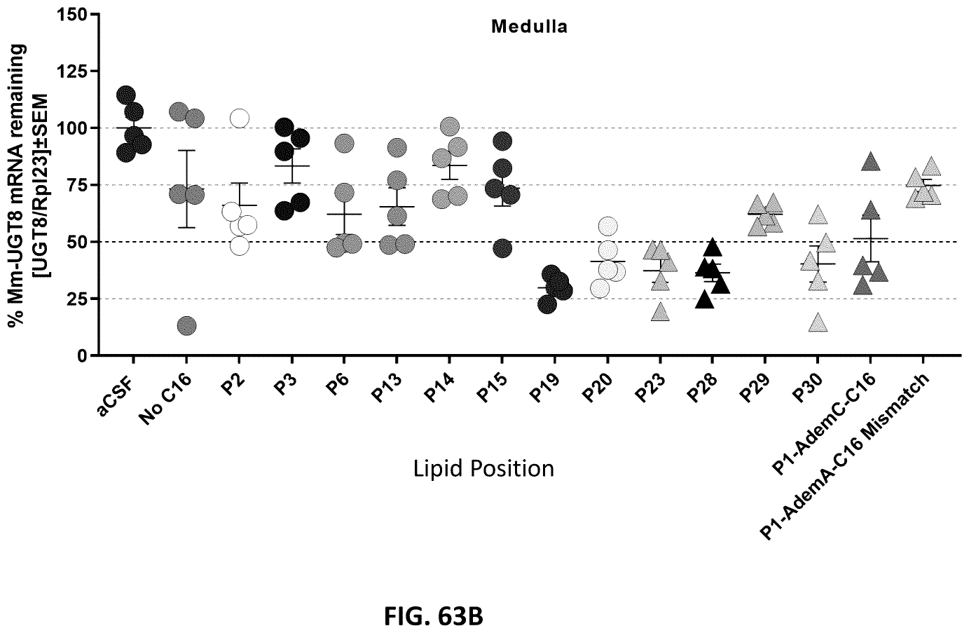

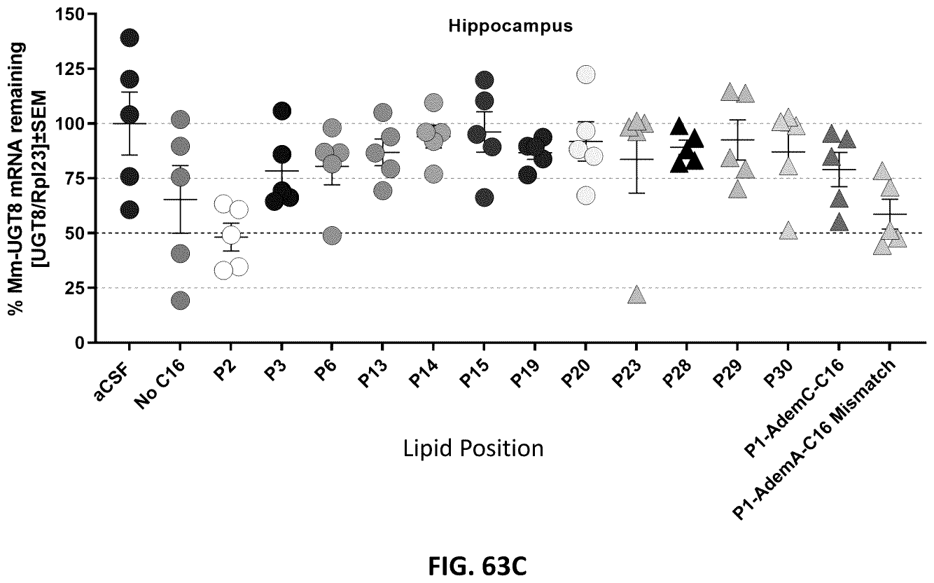

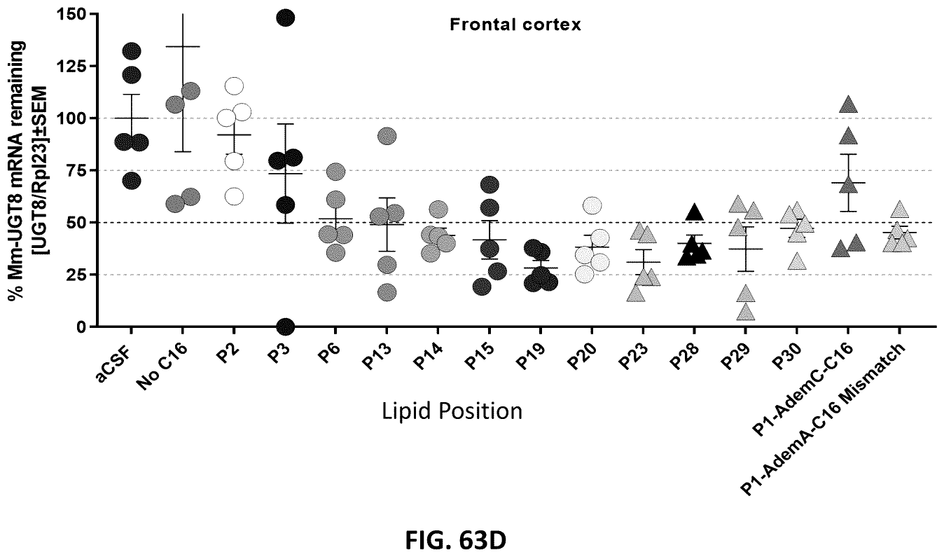

UGT8 mRNA expression was reduced in several tissues of the CNS after administration of UGT8-targeting tetraloop oligonucleotides having a lipid conjugated to a nucleotide of the oligonucleotide (FIGS. 63A-63D). Specifically, lipid conjugation at all positions tested resulted in at least 50% knockdown of the oligodendrocyte-specific mRNA in the lumbar spinal cord, whereas lipid conjugation at P19, P20, P23 or P28 resulted in at least 50% knockdown in the medulla; lipid conjugation at P2 resulted in at least 50% knockdown in the hippocampus; and lipid conjugation at P14, P15, P19, P20, P23, P28, P29 or P30 resulted in at least 50% knockdown in the frontal cortex. These results demonstrate that RNAi oligonucleotide-lipid conjugates comprising a tetraloop reduce exhibit the ability to reduce target (e.g., Ugt8) gene expression in CNS oligodendrocytes. Further, and without wishing to be bound by theory, lipid conjugation near or within the tetraloop provides the most efficient knockdown of a target mRNA expressed in an oligodendrocyte within various regions of the CNS.

### Example 41: Synthesis of Lipid-Conjugated Oligonucleotides

The following schematic depicts the synthesis of a blunt end oligonucleotide with a C16-lipid at the 5′-end. Lipid-conjugated blunt-ended oligonucleotides described herein can be synthesized using post-synthetic methods described in detail in PCT application No. PCT/US2021/42469. Specifically, the oligonucleotides can be synthesized using a post-synthetic conjugation approach such as that depicted below. In Eppendorf tube 1, a solution of palmitic acid in DMA was treated with HATU at rt. In Eppendorf tube 2, a solution of oligo sense strand in H₂O was treated with DIPEA. The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid.

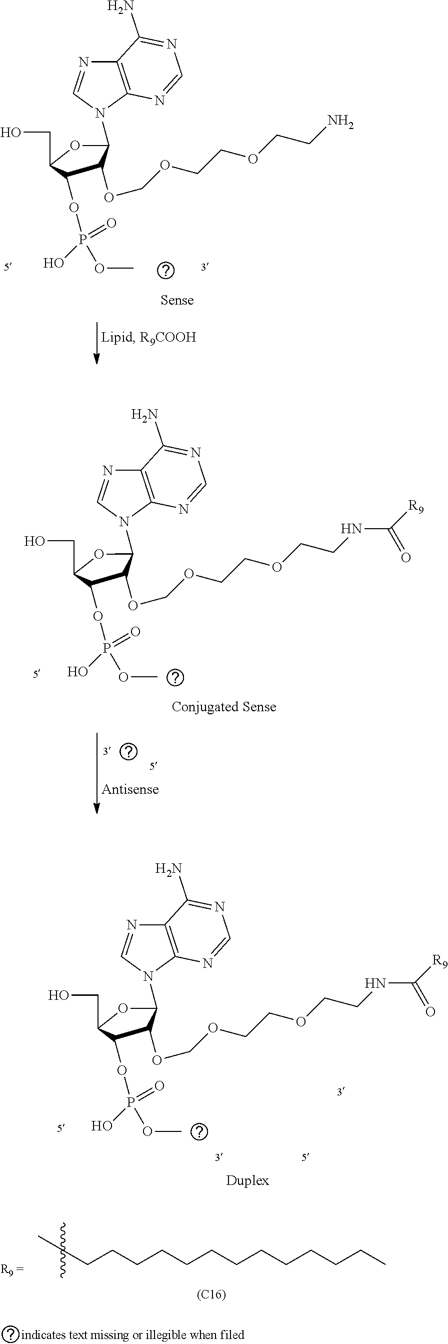

### Example 42: Positional Effects of Lipid Conjugation on the In Vivo Activity of Blunt-End RNAi Oligonucleotide-Lipid Conjugates in the Central Nervous System

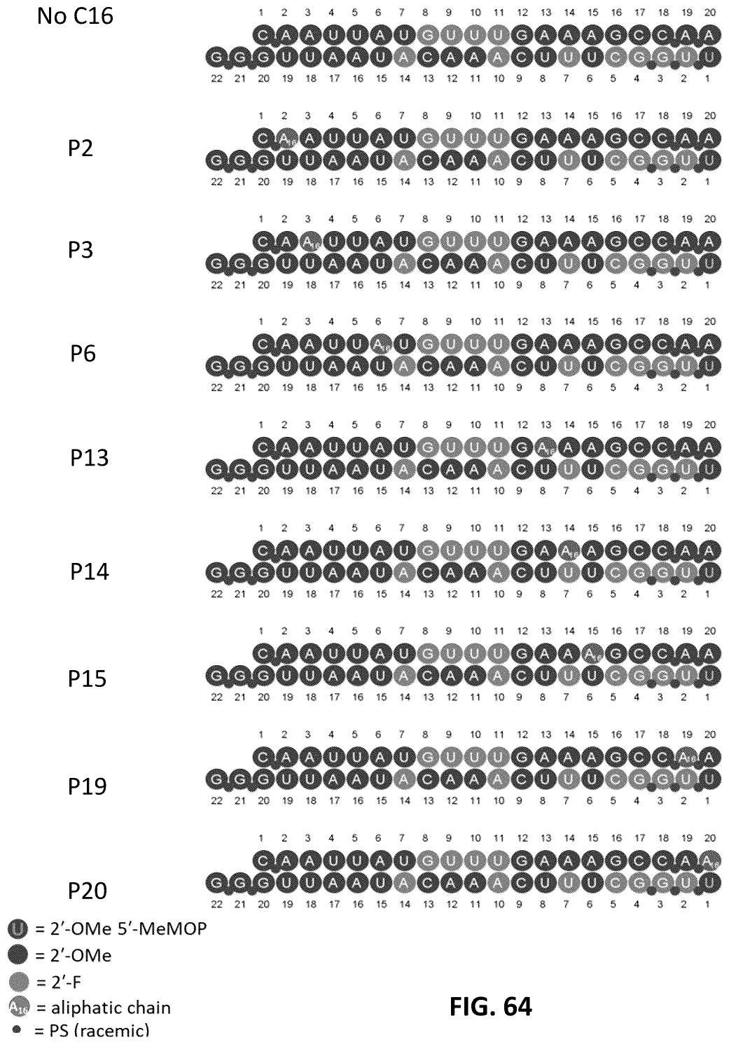

RNAi oligonucleotides conjugated to a lipid at various positions of the sense strand were evaluated for their ability to reduce oligodendrocyte target expression in the CNS. RNAi oligonucleotides conjugated to a C16 lipid were generated by methods described in Example 36. Specifically, oligonucleotides having a blunt-end at the 3′ terminus and a 2-nucleotide overhang at the 5′ terminus were generated with a C16 lipid conjugated at a different position (positions (P) 2, 3, 6, 13, 14, 15, 19, and 20) in the sense strand as shown in the modification patterns below. Each oligonucleotide tested comprised an antisense strand having a region of complementarity to mRNA encoding Ugt8. The unmodified sense and antisense strands are provided in SEQ ID NOs: 856 and 813, respectively (UGT8-277), and the modified strands are shown in Table 36 and FIG. 64.

### Blunt-end RNAi Oligonucleotide Modification Patterns:

No Ligand Sense Strand:
5′-[mXs][mX][mX][mX][mX][mX][mX][fX][fX][fX][fX][mX][mX][mX][mX][mX][mX]
[mXs][mXs][mX]-3′
P2 Sense Strand:
5′-[mXs][ademX-
C16][mX][mX][mX][mX][mX][fX][fX][fX][fX][mX][mX][mX][mX][mX]
[mX][mXs][mXs][mX]-3′
P3 Sense Strand:
5′-[mXs][mX][ademX-
C16][mX][mX][mX][mX][fX][fX][fX][fX][mX][mX][mX][mX][mX]
[mX][mXs][mXs][mX]-3′
P6 Sense Strand:
5′-[mXs][mX][mX][mX][mX][ademX-
C16][mX][fX][fX][fX][fX][mX][mX][mX][mX][mX][mX][mXs][mXs][mX]-3′
P13 Sense Strand:
5′-[mXs][mX][mX][mX][mX][mX][mX][fX][fX][fX][fX][mX][ademX-
C16][mX][mX][mX][mX][mXs][mXs][mX]-3′
P14 Sense Strand:
5′-[mXs][mX][mX][mX][mX][mX][mX][fX][fX][fX][fX][mX][mX][ademX-
C16][mX][mX][mX][mXs][mXs][mX]-3′
P15 Sense Strand:
5′-[mXs][mX][mX][mX][mX][mX][mX][fX][fX][fX][fX][mX][mX][mX][ademX-
C16][mX][mX][mXs][mXs][mX]-3′
P19 Sense Strand:
5′-[mXs][mX][mX][mX][mX][mX][mX][fX][fX][fX][fX][mX][mX][mX][mX][mX]
[mX][mXs][ademXs-C16][mX]-3′
P20 Sense Strand:
5′-[mXs][mX][mX][mX][mX][mX][mX][fX][fX][fX][fX][mX][mX][mX][mX][mX][mX]
[mXs][mXs][ademX-C16]-3′

Each of P2, P3, P6, P13, P14, P15, P19, and P20 hybridized to an antisense strand having the following modification pattern:

Antisense Strand:
5′-[MePhosphonate-4O-mXs][fXs][fXs][fX][fX][mX]
[fX][mX][mX][fX][mX][mX][mX][fX][mX][mX][mX][mX]
[mX][mXs][mXs][mX]-3′

### Modification Kcy: Table 33

TABLE 36
Lipid-conjugated RNAi Blunt-End Oligonucleotides

| | Sense Strand | Antisense Strand |
|---|---|---|
| Oligonucleotide | SEQ ID NO | SEQ ID NO |
| Blunt-End No Ligand | 857 | 840 |
| Blunt-End P2 C16 | 858 | 840 |
| Blunt-End P3 C16 | 859 | 840 |
| Blunt-End P6 C16 | 860 | 840 |
| Blunt-End P13 C16 | 861 | 840 |
| Blunt-End P14 C16 | 862 | 840 |
| Blunt-End P15 C16 | 863 | 840 |
| Blunt-End P19 C16 | 864 | 840 |
| Blunt-End P20 C16 | 865 | 840 |

To evaluate the oligonucleotides in Table 36, C57BL/6 female mice, 6-8 weeks old, were treated with 300 μg of blunt-end RNAi oligonucleotide formulated in artificial cerebrospinal fluid (aCSF) via intrathecal (i.t.) lumbar injection. Target knockdown was assessed 7 days after injection.

RNA was extracted from tissue samples from the lumbar spinal cord, medulla, hippocampus, and frontal cortex to determine murine Ugt8 mRNA levels by qPCR as described in Example 37.

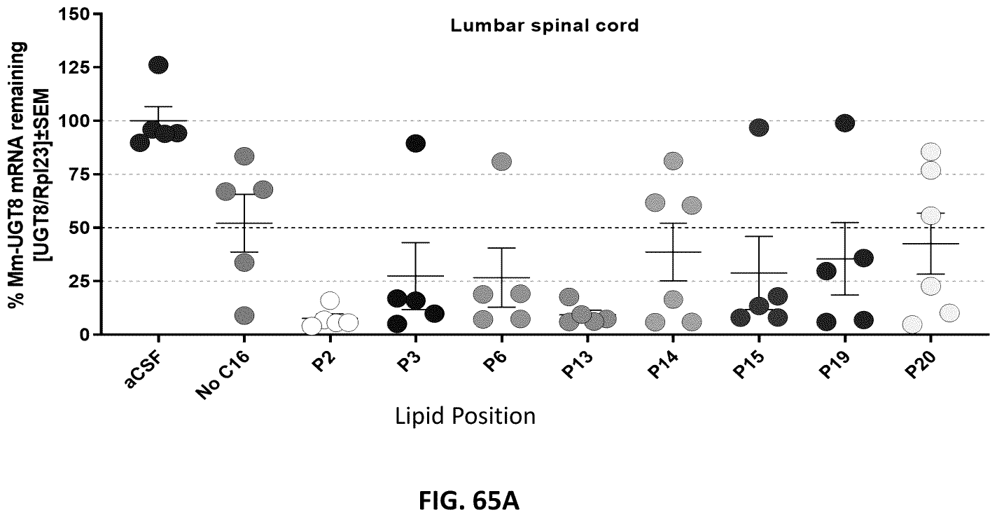

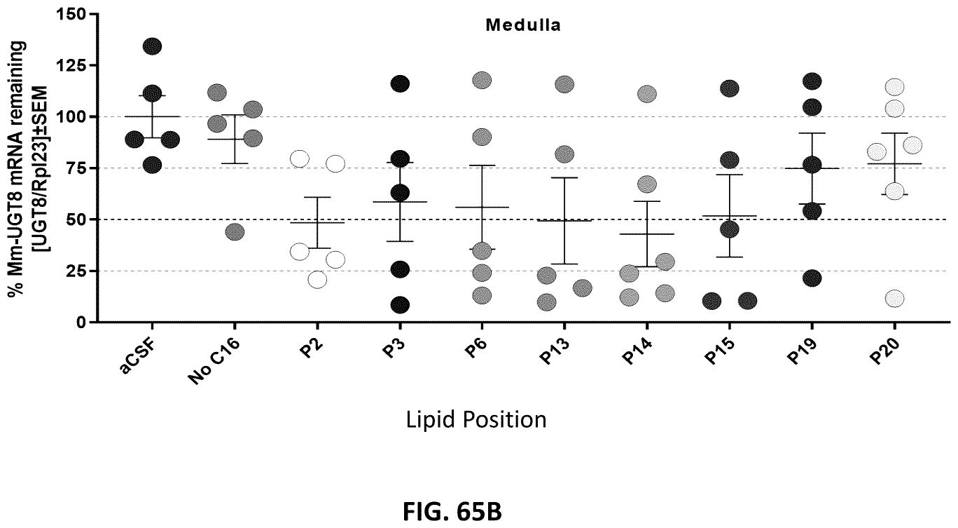

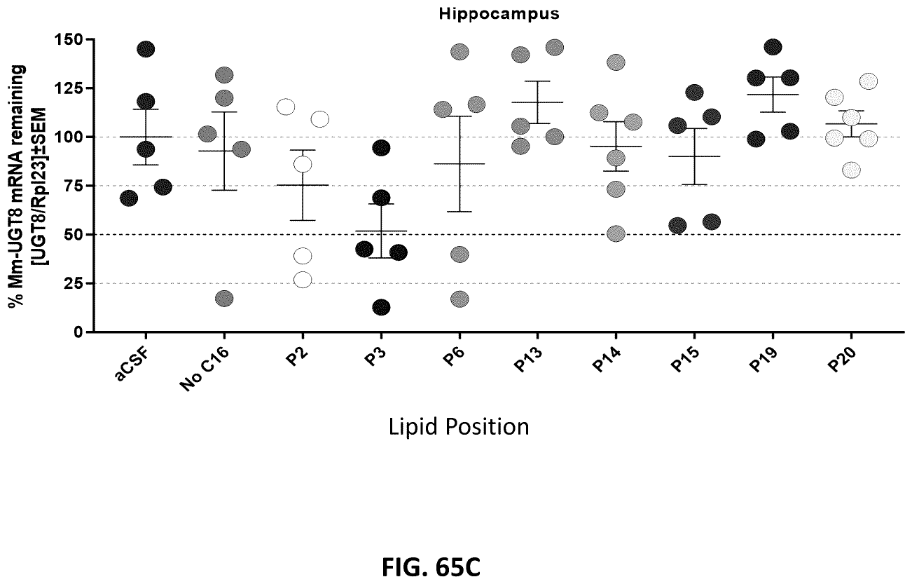

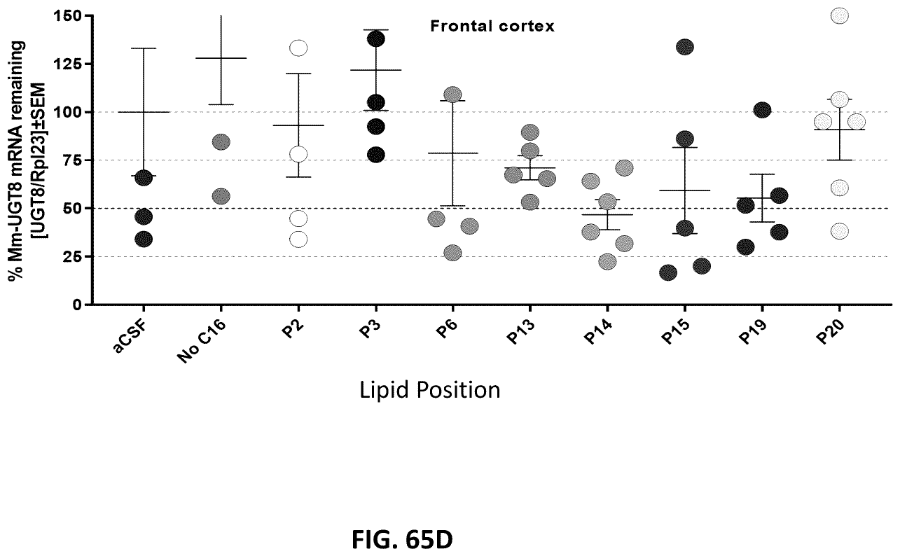

Ugt8 mRNA expression was reduced in several tissues of the CNS after administration of UGT8-targeting blunt-end RNAi oligonucleotide lipid-conjugates (FIGS. 65A-65D). Specifically, lipid conjugation at all positions tested resulted in at least 50% knockdown of the oligodendrocyte-specific mRNA in the lumbar spinal cord, whereas lipid conjugation at P2, P14 or P15 resulted in at least 50% knockdown in the medulla; lipid conjugation at P3 resulted in at least 50% knockdown in the hippocampus; and lipid conjugation at P14 resulted in at least 50% knockdown in the frontal cortex.

### Example 43: Positional Effects of Lipid Conjugation on the In Vivo Activity of Blunt-End RNAi Oligonucleotide-Lipid Conjugates in the Central Nervous System

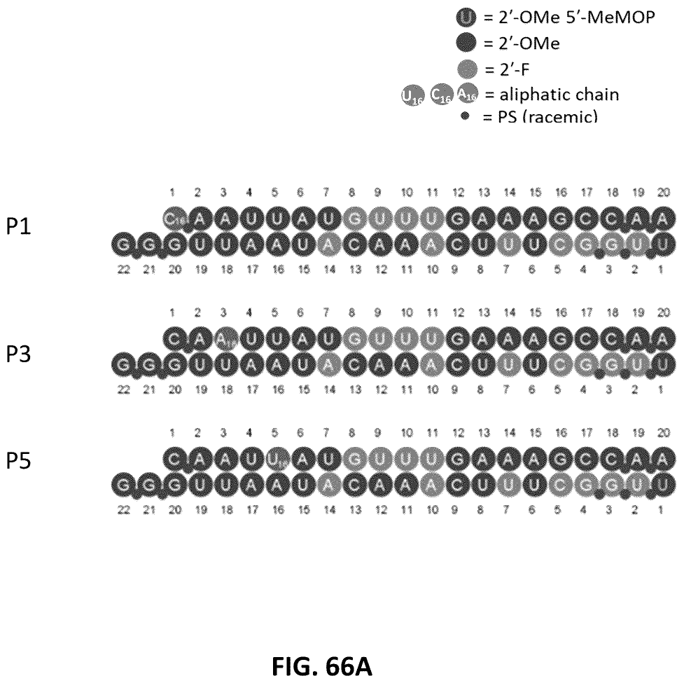

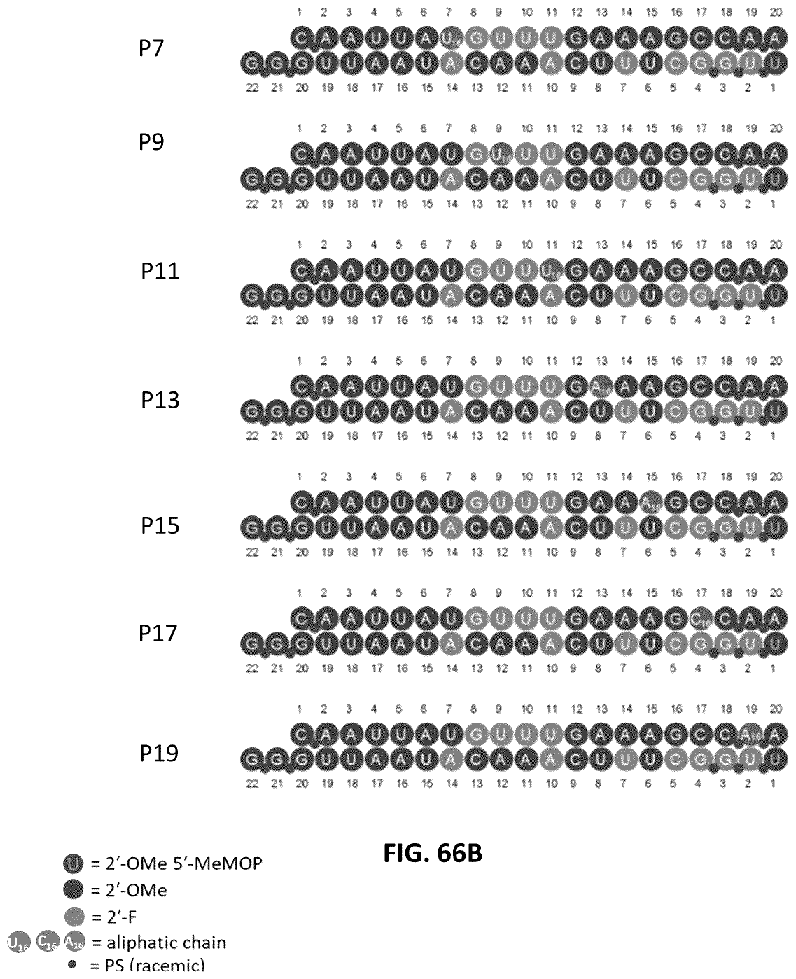

Following the observation that oligodendrocyte-specific mRNA is reduced in the CNS after administration of lipid-conjugated RNAi oligonucleotides, additional lipid conjugation positions were evaluated in a long-term 28-day study. RNAi oligonucleotides conjugated to a C16 lipid were generated by methods described in Example 36. Specifically, oligonucleotides having a blunt-end at the 3′ terminus and a 2-nucleotide overhang at the 5′ terminus were generated with a C16 lipid conjugated at a different position (positions (P) 1, 3, 5, 7, 9, 11, 13, 15, 17, and 19) in the sense strand as shown in the modification patterns below and in FIGS. 66A-66B. Each oligonucleotide tested comprised an antisense strand having a region of complementarity to mRNA encoding Ugt8. The unmodified sense and antisense strands are provided in SEQ ID NOs: 856 and 813, respectively (UGT8-277), and the modified strands are shown in Table 37.

### Blunt-End RNAi Oligonucleotide Modification Patterns:

P1 Sense Strand:
5′-[ademXs-
C16][mX][mX][mX][mX][mX][mX][fX][fX][fX][fX][mX][mX][mX][mX][mX]
[mX][mXs][mXs][mX]-3′
P3 Sense Strand:
5′-[mXs][mX][ademX-
C16][mX][mX][mX][mX][fX][fX][fX][fX][mX][mX][mX][mX][mX]
[mX][mXs][mXs][mX]-3′
P5 Sense Strand:
5′-[mXs][mX][mX][mX][ademX-
C16][mX][mX][mX][fX][fX][fX][fX][mX][mX][mX][mX][mX][mXs][mXs][mX]-3′
P7 Sense Strand:
5′-[mXs][mX][mX][mX][mX][mX][ademX-
C16][fX][fX][fX][fX][mX][mX][mX][mX][mX][mX][mXs][mXs][mX]-3′
P9 Sense Strand:
5′-[mXs][mX][mX][mX][mX][mX][mX][fX][ademX-
C16][fX][fX][mX][mX][mX][mX][mX][X][mXs][mXs][mX]-3′
P11 Sense Strand:
5′-[mXs][mX][mX][mX][mX][mX][mX][fX][fX][fX][ademX-
C16][mX][mX][mX][mX][mX][mX][mXs][mXs][mX]-3′
P13 Sense Strand:
5′-[mXs][mX][mX][mX][mX][mX][mX][fX][fX][fX][fX][mX][ademX-
C16][mX][mX][mX][mX][mXs][mXs][mX]-3′
P15 Sense Strand:
5′-[mXs][mX][mX][mX][mX][mX][mX][fX][fX][fX][fX][mX][mX][mX][ademX-
C16][mX][mX][mXs][mXs][mX]-3′
P17 Sense Strand:
5′-[mXs][mX][mX][mX][mX][mX][mX][fX][fX][fX][fX][mX][mX][mX][mX][mX]
[ademX-C16][mXs][mXs][mX]-3′
P19 Sense Strand:
5′-[mXs][mX][mX][mX][mX][mX][mX][fX][fX][fX][fX][mX][mX][mX][mX][mX]
[mX][mXs][ademXs-C16][mX]-3′

Each of P1, P3, P5, P7, P9, P11, P13, P15, P17, and P19 hybridized to an antisense strand having the following modification pattern:

Antisense Strand:
5′-[MePhosphonate-4O-mXs][fXs][fXs][fX][fX][mX]
[mX][mX][mX][fX][mX][mX][mX][fX][mX][mX][mX][mX]
[mX][mXs][mXs][mX]-3′

### Modification Key: Table 36

TABLE 37
Lipid-conjugated RNAi Blunt-End Oligonucleotides

| | Sense Strand | Antisense Strand |
|---|---|---|
| Oligonucleotide | SEQ ID NO | SEQ ID NO |
| Blunt-End P1 C16 | 867 | 840 |
| Blunt-End P3 C16 | 859 | 840 |
| Blunt-End P5 C16 | 868 | 840 |
| Blunt-End P7 C16 | 869 | 840 |
| Blunt-End P9 C16 | 870 | 840 |
| Blunt-End P11 C16 | 871 | 840 |
| Blunt-End P13 C16 | 861 | 840 |
| Blunt-End P15 C16 | 863 | 840 |
| Blunt-End P17 C16 | 872 | 840 |
| Blunt-End P19 C16 | 864 | 840 |

To evaluate the oligonucleotides in Table 37, C57BL/6 female mice, 6-8 weeks old, were treated with 300 μg of blunt-end RNAi oligonucleotide formulated in artificial cerebrospinal fluid (aCSF) via intrathecal (i.t.) lumbar injection. Target knockdown was assessed 28 days after injection.

RNA was extracted from tissue samples from the lumbar spinal cord, brain stem, hypothalamus, hippocampus, and frontal cortex to determine murine Ugt8 mRNA levels by qPCR as described in Example 37.

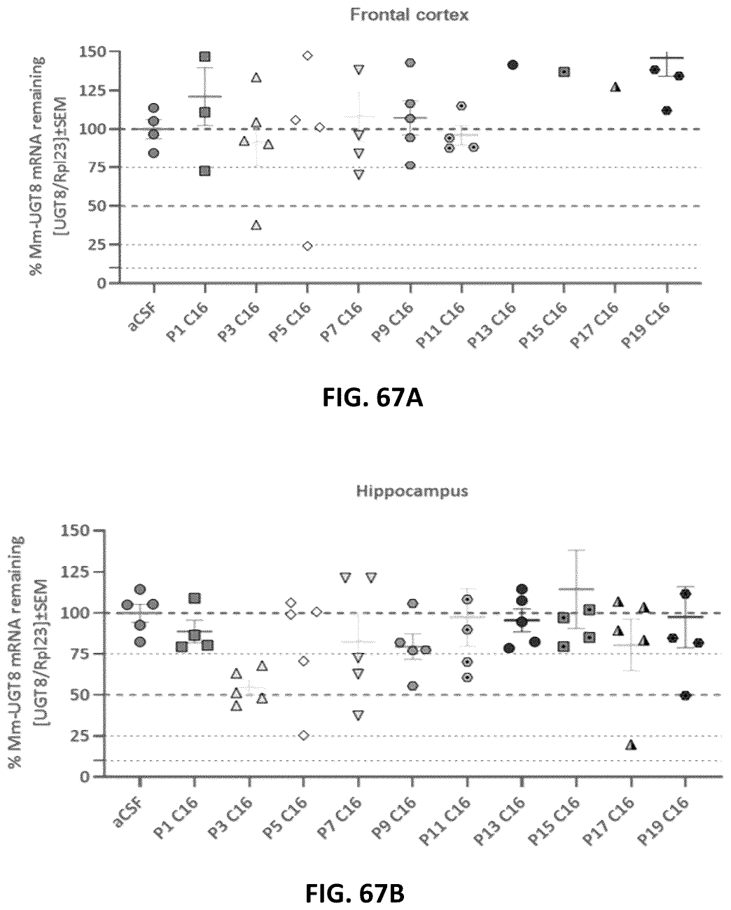

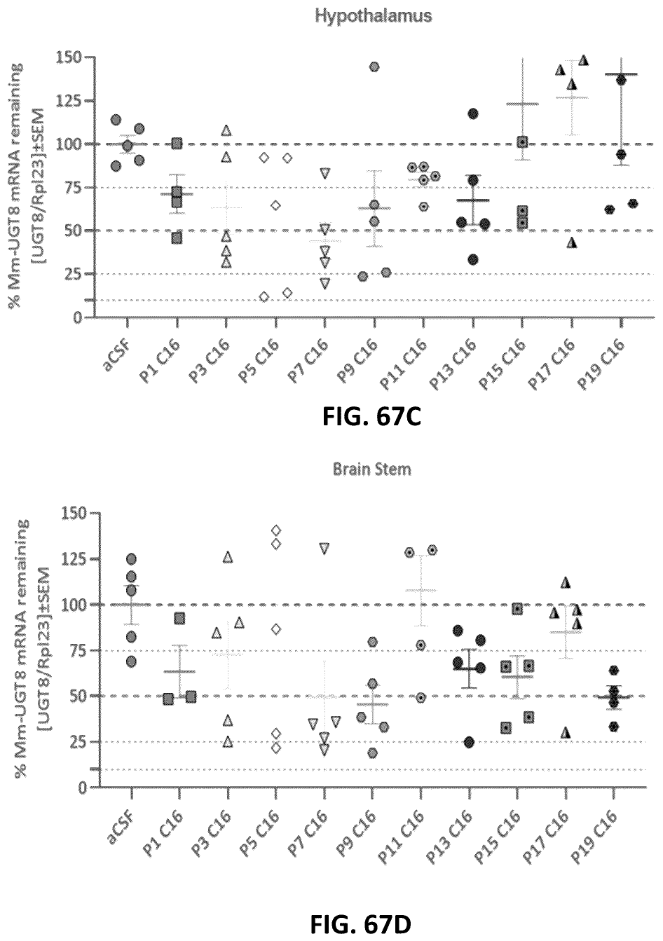

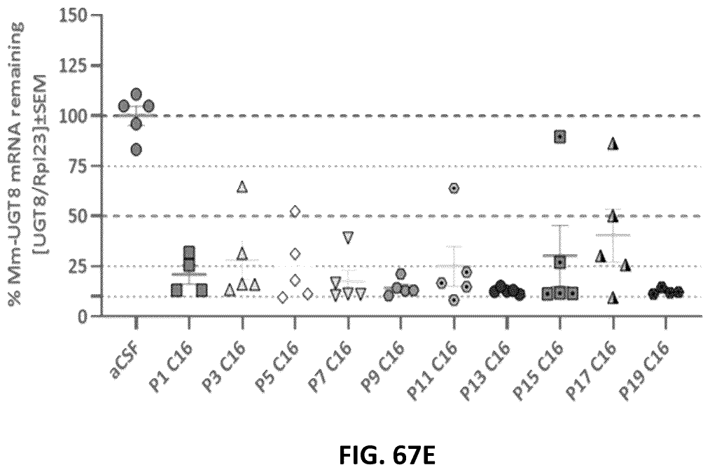

Ugt8 mRNA expression was reduced in several tissues of the CNS after administration of UGT8-targeting blunt-end RNAi oligonucleotide lipid-conjugates (FIGS. 67A-67E). Specifically, lipid conjugation at all positions tested resulted in at least 50% knockdown of the oligodendrocyte-specific mRNA in the lumbar spinal cord. Lipid conjugation at P3, P5, P7 and P9 resulted in knockdown throughout the CNS, and lipid conjugation at P3 resulted in about a 50% knockdown in the hippocampus.

SEQUENCE LISTING-I

| | | | SEQ |
|---|---|---|---|
| Name | Strand | Sequence | ID NO |
| Pecam1- | Sense | ACAGAUACUCUAGAACGGAAGCAGCCGAAAGGCUG | 1 |
| 2392 | | C | |
| Pecam1- | Sense | AUUUUGUGUACUAUACCUAAGCAGCCGAAAGGCUG | 2 |
| 3222 | | C | |
| Cd68- | Sense | AGGCGCAGAAUUCAUCUCUAGCAGCCGAAAGGCUG | 3 |
| 0815 | | C | |
| Pecam1- | Anti- | UUCCGUUCUAGAGUAUCUGUGG | 4 |
| 2392 | sense | | |
| Pecam1- | Anti- | UUAGGUAUAGUACACAAAAUGG | 5 |
| 3222 | sense | | |
| Cd68- | Anti- | UAGAGAUGAAUUCUGCGCCUGG | 6 |
| 0815 | sense | | |
| Pecam1- | Sense | [ademAs-C22][mC][mA][mG][mA][mU][mA][fC][fU] | 7 |
| 2392 | | [fC][fU][mA][mG][mA][mA][mC][mG][mG][mA][mA][mG] | |
| | | [mC][mA][mG][mC][mC][mG][mA][mA][mA][mG][mG][mC] | |
| | | [mU][mG][mC] | |
| Pecam1- | Sense | [ademAs-C22][mU][mU][mU][mU][mG][mU][fG] | 8 |
| 3222 | | [fU][fA][fC][mU][mA][mU][mA][mC][mC][mU][mA][mA] | |
| | | [mG][mC][mA][mG][mC][mC][mG][mA][mA][mA][mG][mG] | |
| | | [mC][mU][mG][mC] | |
| Cd68- | Sense | [ademAs-C22][mG][mG][mC][mG][mC][mA][fG] | 9 |
| 0815 | | [fA][fA][fU][mU][mC][mA][mU][mC][mU][mC][mU][mA] | |
| | | [mG][mC][mA][mG][mC][mC][mG][mA][mA][mA][mG][mG] | |
| | | mC][mU][mG][mC] | |
| Pecam1- | Anti- | [MePhosphonate-4O-mUs][fUs][fCs][fC][fG][mU] | 10 |
| 2392 | sense | [fU][mC][mU][fA][mG][mA][mG][fU][mA][mU][mC][mU] | |
| | | [mG][mUs][mGs][mG] | |
| Pecam1- | Anti- | [MePhosphonate-4O-mUs][fUs][fAs][fG][fG][mU] | 11 |
| 3222 | sense | [fA][mU][mA][fG][mU][mA][mC][fA][mC][mA][mA][mA] | |
| | | [mA][mUs][mGs][mG] | |
| Cd68- | Anti- | [MePhosphonate-4O-mUs][fAs][fGs][fA][fG][mA][fU] | 12 |
| 0815 | sense | [mG][mA][fA][mU][mU][mC][fU][mG][mC][mG][mC][mC] | |
| | | [mUs][mGs][mG] | |
| Cd68- | Sense | AGGCGCAGAAUUCAUCUCUAGCAGCCGAAAGGCUG | 13 |
| 0815 | | C | |
| Cd68- | Sense | AGGCGCAGAAUUCAUCUCUA | 14 |
| 0815 | | | |
| Cd68- | Sense | GGCGCAGAAUUCAUCUCUA | 15 |
| 0815 | | | |
| Cd68- | Sense | GCGCAGAAUUCAUCUCUA | 16 |
| 0815 | | | |
| Cd68- | Sense | CGCAGAAUUCAUCUCUA | 17 |
| 0815 | | | |
| Cd68- | Sense | GCAGAAUUCAUCUCUA | 18 |
| 0815 | | | |
| Cd68- | Sense | CAGAAUUCAUCUCUA | 19 |
| 0815 | | | |
| Cd68- | Sense | GGCGCAGAAUUCAUCUC | 20 |
| 0815 | | | |
| Cd68- | Sense | GCGCAGAAUUCAUCUC | 21 |
| 0815 | | | |
| Cd68- | Sense | CGCAGAAUUCAUCUC | 22 |
| 0815 | | | |
| Cd68- | Sense | GCAGAAUUCAUCUC | 23 |
| 0815 | | | |
| Cd68- | Sense | CAGAAUUCAUCUC | 24 |
| 0815 | | | |
| Cd68- | Sense | GGCGCAGAAUUCAUCUC | 25 |
| 0815 | | | |
| Cd68- | Sense | GCGCAGAAUUCAUCUC | 26 |
| 0815 | | | |
| Cd68- | Sense | CGCAGAAUUCAUCUC | 27 |
| 0815 | | | |
| Cd68- | Sense | UAGAGAUGAAUUCUGCGCCUGG | 28 |
| 0815 | | | |
| Cd68- | Sense | UAGAGAUGAAUUCUGCGCCUGG | 29 |
| 0815 | | | |
| Cd68- | Sense | UAGAGAUGAAUUCUGCGCCUGG | 30 |
| 0815 | | | |
| Cd68- | Sense | UAGAGAUGAAUUCUGCGCCUGG | 31 |
| 0815 | | | |
| Cd68- | Sense | CGCAGAAUUCAUCUC | 32 |
| 0815 | | | |
| Cd68- | Sense | GCAGAAUUCAUCUC | 33 |
| 0815 | | | |
| Cd68- | Sense | CAGAAUUCAUCUC | 34 |
| 0815 | | | |
| Cd68- | Sense | GGCGCAGAAUUCATCUC | 35 |
| 0815 | | | |
| Cd68- | Sense | GCGCAGAAUUCATCUC | 36 |
| 0815 | | | |
| Cd68- | Sense | CGCAGAAUUCATCUC | 37 |
| 0815 | | | |
| Cd68- | Sense | GCAGAAUUCATCUC | 38 |
| 0815 | | | |
| Cd68- | Sense | CAGAAUUCATCUC | 39 |
| 0815 | | | |
| Cd68- | Sense | GGCGCAGAAUUCATCU | 40 |
| 0815 | | | |
| Cd68- | Sense | GCGCAGAAUUCATCU | 41 |
| 0815 | | | |
| Cd68- | Sense | CGCAGAAUUCATCU | 42 |
| 0815 | | | |
| Cd68- | Sense | GCAGAAUUCATCU | 43 |
| 0815 | | | |
| Cd68- | Sense | CAGAAUUCATCU | 44 |
| 0815 | | | |
| Cd68- | Sense | GGCGCAGAAUUCATC | 45 |
| 0815 | | | |
| Cd68- | Sense | GCGCAGAAUUCATC | 46 |
| 0815 | | | |
| Cd68- | Sense | CGCAGAAUUCATC | 47 |
| 0815 | | | |
| Cd68- | Sense | GCAGAAUUCATC | 48 |
| 0815 | | | |
| Cd68- | Sense | CAGAAUUCATC | 49 |
| 0815 | | | |
| Cd68- | Sense | AGAAUUCATC | 50 |
| 0815 | | | |
| Cd68- | Sense | GAAUUCATC | 51 |
| 0815 | | | |
| Cd68- | Sense | AAUUCATC | 52 |
| 0815 | | | |
| Cd68- | Sense | AGGCGCAGAAUUCAUCUCUA | 53 |
| 0815 | | | |
| Cd68- | Sense | CAGAAUUCAUCUCUAGCAGCCGAAAGGCUGC | 54 |
| 0815 | | | |
| Cd68- | Sense | CAGAAUUCAUCUCUAGCAGCCGAAAGGCUGC | 55 |
| 0815 | | | |
| Cd68- | Sense | CAGAAUUCAUCUCUAGCAGCCGAAAGGCUGC | 56 |
| 0815 | | | |
| Cd68- | Sense | CAGAAUUCAUCUCUAGCAGCCGAAAGGCUGC | 57 |
| 0815 | | | |
| Cd68- | Anti- | UAGAGAUGAAUUCUGCGCCUGG | 58 |
| 0815 | sense | | |
| Cd68- | Anti- | UAGAGAUGAAUUCUGCGCCUGG | 59 |
| 0815 | sense | | |
| Cd68- | Anti- | UAGAGAUGAAUUCUGCGCCUGG | 60 |
| 0815 | sense | | |
| Cd68- | Anti- | UAGAGAUGAAUUCUGCGCCUGG | 61 |
| 0815 | sense | | |
| Cd68- | Anti- | UAGAGAUGAAUUCUGCGCCUGG | 62 |
| 0815 | sense | | |
| Cd68- | Anti- | UAGAGAUGAAUUCUGCGCCUGG | 63 |
| 0815 | sense | | |
| Cd68- | Anti- | UAGAGAUGAAUUCUGCGCCUGG | 64 |
| 0815 | sense | | |
| Cd68- | Anti- | UAGAGAUGAAUUCUGCGCCUGG | 65 |
| 0815 | sense | | |
| Cd68- | Anti- | UAGAGAUGAAUUCUGCGCCUGG | 66 |
| 0815 | sense | | |
| Cd68- | Anti- | UAGAGAUGAAUUCUGCGCCUGG | 67 |
| 0815 | sense | | |
| Cd68- | Anti- | UAGAGAUGAAUUCUGCGCCUGG | 68 |
| 0815 | sense | | |
| Cd68- | Anti- | UAGAGAUGAAUUCUGCGCCUGG | 69 |
| 0815 | sense | | |
| Cd68- | Anti- | UAGAGAUGAAUUCUGCGCCUGG | 70 |
| 0815 | sense | | |
| Cd68- | Anti- | UAGAGAUGAAUUCUGCGCCUGG | 71 |
| 0815 | sense | | |
| Cd68- | Anti- | UAGAGAUGAAUUCUGCGCCUGG | 72 |
| 0815 | sense | | |
| Cd68- | Anti- | UAGAGAUGAAUUCUGCGCCUGG | 73 |
| 0815 | sense | | |
| Cd68- | Anti- | UAGAGAUGAAUUCUGCGCCUGG | 74 |
| 0815- | sense | | |
| Cd68- | Anti- | UAGAGAUGAAUUCUGCGCCUGG | 75 |
| 0815 | sense | | |
| Cd68- | Anti- | UAGAGAUGAAUUCUGCGCCUGG | 76 |
| 0815 | sense | | |
| Cd68- | Anti- | UAGAGAUGAAUUCUGCGCCUGG | 77 |
| 0815 | sense | | |
| Cd68- | Anti- | UAGAGAUGAAUUCUGCGCCUGG | 78 |
| 0815 | sense | | |
| Cd68- | Anti- | UAGAGAUGAAUUCUGCGCCUGG | 79 |
| 0815 | sense | | |
| Cd68- | Anti- | UAGAGAUGAAUUCUGCGCCUGG | 80 |
| 0815 | sense | | |
| Cd68- | Anti- | UAGAGAUGAAUUCUGCGCCUGG | 81 |
| 0815 | sense | | |
| Cd68- | Anti- | UAGAGAUGAAUUCUGCGCCUGG | 82 |
| 0815 | sense | | |
| Cd68- | Anti- | UAGAGAUGAAUUCUGCGCCUGG | 83 |
| 0815 | sense | | |
| Cd68- | Anti- | UAGAGAUGAAUUCUGCGCCUGG | 84 |
| 0815 | sense | | |
| Cd68- | Anti- | UAGAGAUGAAUUCUGCGCCUGG | 85 |
| 0815 | sense | | |
| Cd68- | Anti- | UAGAGAUGAAUUCUGCGCCUGG | 86 |
| 0815 | sense | | |
| Cd68- | Anti- | UAGAGAUGAAUUCUGCGCCUGG | 87 |
| 0815 | sense | | |
| Cd68- | Anti- | UAGAGAUGAAUUCUGCGCCUGG | 88 |
| 0815 | sense | | |
| Cd68- | Anti- | UAGAGAUGAAUUCUGCGCCUGG | 89 |
| 0815 | sense | | |
| Cd68- | Anti- | UAGAGAUGAAUUCUGCGCCUGG | 90 |
| 0815 | sense | | |
| Cd68- | Anti- | UAGAGAUGAAUUCUGCGCCUGG | 91 |
| 0815 | sense | | |
| Cd68- | Anti- | UAGAGAUGAAUUCUGCGCCUGG | 92 |
| 0815 | sense | | |
| Cd68- | Anti- | UAGAGAUGAAUUCUGCGCCUGG | 93 |
| 0815 | sense | | |
| Cd68- | Anti- | UAGAGAUGAAUUCUGCGCCUGG | 94 |
| 0815 | sense | | |
| Cd68- | Anti- | UAGAGAUGAAUUCUGCGCCUGG | 95 |
| 0815 | sense | | |
| Cd68- | Anti- | UAGAGAUGAAUUCUGCGCCUGG | 96 |
| 0815 | sense | | |
| Cd68- | Anti- | UAGAGAUGAAUUCUGCGCCUGG | 97 |
| 0815 | sense | | |
| Cd68- | Anti- | UAGAGAUGAAUUCUGCGCCUGG | 98 |
| 0815 | sense | | |
| Cd68- | Anti- | UAGAGAUGAAUUCUGCGCCUGG | 99 |
| 0815 | sense | | |
| Cd68- | Anti- | UAGAGAUGAAUUCUGCGCCUGG | 100 |
| 0815 | sense | | |
| Cd68- | Anti- | UAGAGAUGAAUUCUGCGCCUGG | 101 |
| 0815 | sense | | |
| Cd68- | Anti- | UAGAGAUGAAUUCUGCGCCUGG | 102 |
| 0815 | sense | | |
| Cd68- | Sense | [mAs][mG][mG][mC][mG][mC][mA][fG][fA][fA][fU][mU] | 103 |
| 0815 | | [mC][mA][mU][mC][mU][mC][mU][mA][mG][mC][mA][mG] | |
| | | [mC][mC][mG][ademA-C22][mA][mA][mG] | |
| | | [mG][mC][mU][mG][mC] | |
| Cd68- | Sense | [mAs][mG][mG][mC][mG][mC][ademA-C22][fG][fA] | 104 |
| 0815 | | [fA][fU][mU][mC][mA][mU][mC][mU][mCs][mUs][mA] | |
| Cd68- | Sense | [mGs][mG][mC][mG][mC][ademA-C22][fG][A][fA][fU] | 105 |
| 0815 | | [mU][mC][mA][mU][mC][mU][mCs][mUs][mA] | |
| Cd68- | Sense | [mGs][mC][mG][mC][ademA-C22][fG][fA][fA][fU][mU] | 106 |
| 0815 | | [mC][mA][mU][mC][mU][mCs][mUs][mA] | |
| Cd68- | Sense | [mCs][mG][mC][ademA-C22][fG][A][fA][fU] | 107 |
| 0815 | | [mU][mC][mA][mU][mC][mU][mCs][mUs][mA] | |
| Cd68- | Sense | [mGs][mC][ademA-C22][fG][fA][fA][fU][mU] | 108 |
| 0815 | | [mC][mA][mU][mC][mU][mCs][mUs][mA] | |
| Cd68- | Sense | [mCs][ademA-C22][fG][fA][fA][fU][mU][mC] | 109 |
| 0815 | | [mA][mU][mC][mU][mCs][mUs][mA] | |
| Cd68- | Sense | [mGs][mG][mC][mG][mC][ademA-C22][fG][fA][fA][fU] | 110 |
| 0815 | | [mU][mC][mA][mU][mCs][mUs][mC] | |
| Cd68- | Sense | [mGs][mC][mG][mC][ademA-C22][fG][fA][fA][fU] | 111 |
| 0815 | | [mU][mC][mA][mU][mCs][mUs][mC] | |
| Cd68- | Sense | [mCs][mG][mC][ademA-C22][fG][fA][fA][fU] | 112 |
| 0815 | | [mU][mC][mA][mU][mCs][mUs][mC] | |
| Cd68- | Sense | [mGs][mC][ademA-C22][fG][fA][fA][fU][mU][mC] | 113 |
| 0815 | | [mA][mU][mCs][mUs][mC] | |
| Cd68- | Sense | [mCs][ademA-C22][fG][fA][fA][fU][mU][mC][mA][mU] | 114 |
| 0815 | | [mCs][mUs][mC] | |
| Cd68- | Sense | [+Gs][mG][mC][mG][mC][ademA-C22][fG][fA][fA][fU] | 115 |
| 0815 | | [mU][mC][mA][mU][mCs][mUs][mC] | |
| Cd68- | Sense | [+Gs][mC][mG][mC][ademA- | 116 |
| 0815 | | C22][fG][fA][fA][fU][mU][mC][mA][mU][mCs][mUs][mC] | |
| Cd68- | Sense | [+Cs][mG][mC][ademA- | 117 |
| 0815 | | C22][fG][fA][fA][fU][mU][mC][mA][mU][mCs][mUs][mC] | |
| Cd68- | Sense | [+Gs][mC][ademA- | 118 |
| 0815 | | C22][fG][A][fA][fU][mU][mC][mA][mU][mCs][mUs][mC] | |
| Cd68- | Sense | [+Cs][ademA- | 119 |
| 0815 | | C22][fG][fA][fA][fU][mU][mC][mA][mU][mCs][mUs][mC] | |
| Cd68- | Sense | [+Gs][mG][mC][mG][mC][ademA- | 120 |
| 0815 | | C22][fG][fA][fA][fU][mU][mC][mA][mU][+Cs][mUs][mC] | |
| Cd68- | Sense | [+Gs][mC][mG][mC][ademA- | 121 |
| 0815 | | C22][fG][fA][fA][fU][mU][mC][mA][mU][+Cs][mUs][mC] | |
| Cd68- | Sense | [+Cs][mG][mC][ademA- | 122 |
| 0815 | | C22][fG][fA][fA][fU][mU][mC][mA][mU][+Cs][mUs][mC] | |
| Cd68- | Sense | [+Gs][mC][ademA- | 123 |
| 0815 | | C22][fG][fA][fA][fU][mU][mC][mA][mU][+Cs][mUs][mC] | |
| Cd68- | Sense | [+Cs][ademA- | 124 |
| 0815 | | C22][fG][fA][fA][fU][mU][mC][mA][mU][+Cs][mUs][mC] | |
| Cd68- | Sense | [+Gs][mG][mC][mG][mC][ademA- | 125 |
| 0815 | | C22][fG][fA][fA][fU][mU][mC][mA][+T][+Cs][mUs][mC] | |
| Cd68- | Sense | [+Gs][mC][mG][mC][ademA- | 126 |
| 0815 | | C22][fG][fA][fA][fU][mU][mC][mA][+T][+Cs][mUs][mC] | |
| Cd68- | Sense | [+Cs][mG][mC][ademA- | 127 |
| 0815 | | C22][fG][fA][fA][fU][mU][mC][mA][+T][+Cs][mUs][mC] | |
| Cd68- | Sense | [+Gs][mC][ademA- | 128 |
| 0815 | | C22][fG][fA][fA][fU][mU][mC][mA][+T][+Cs][mUs][mC] | |
| Cd68- | Sense | [+Cs][ademA- | 129 |
| 0815 | | C22][fG][fA][fA][fU][mU][mC][mA][+T][+Cs][mUs][mC] | |
| Cd68- | Sense | [+Gs][mG][mC][mG][mC][ademA- | 130 |
| 0815 | | C22][fG][fA][fA][fU][mU][mC][mA][+Ts][+Cs][mU] | |
| Cd68- | Sense | [+Gs][mC][mG][mC][ademA- | 131 |
| 0815 | | C22][fG][fA][fA][fU][mU][mC][mA][+Ts][+Cs][mU] | |
| Cd68- | Sense | [+Cs][mG][mC][ademA- | 132 |
| 0815 | | C22][fG][fA][fA][fU][mU][mC][mA][+Ts][+Cs][mU] | |
| Cd68- | Sense | [+Gs][mC][ademA- | 133 |
| 0815 | | C22][fG][fA][fA][fU][mU][mC][mA][+Ts][+Cs][mU] | |
| Cd68- | Sense | [+Cs][ademA- | 134 |
| 0815 | | C22][fG][fA][fA][fU][mU][mC][mA][+Ts][+Cs][mU] | |
| Cd68- | Sense | [+Gs][mG][mC][mG][mC][ademA- | 135 |
| 0815 | | C22][fG][fA][fA][fU][mU][mC][mAs][+Ts][+C] | |
| Cd68- | Sense | [+Gs][mC][mG][mC][ademA- | 136 |
| 0815 | | C22][fG][fA][fA][fU][mU][mC][mAs][+Ts][+C] | |
| Cd68- | Sense | [+Cs][mG][mC][ademA- | 137 |
| 0815 | | C22][fG][fA][fA][fU][mU][mC][mAs][+Ts][+C] | |
| Cd68- | Sense | [+Gs][mC][ademA- | 138 |
| 0815 | | C22][fG][fA][fA][fU][mU][mC][mAs][+Ts][+C] | |
| Cd68- | Sense | [+Cs][ademA- | 139 |
| 0815 | | C22][fG][fA][fA][fU][mU][mC][mAs][+Ts][+C] | |
| Cd68- | Sense | [+As][fG][fA][ademA-C22][fU][mU][mC][mAs][+Ts][+C] | 140 |
| 0815 | | | |
| Cd68- | Sense | [+Gs][fA][ademA-C22][fU][mU][mC][mAs][+Ts][+C] | 141 |
| 0815 | | | |
| Cd68- | Sense | [+As][ademA-C22][fU][mU][mC][mAs][+Ts][+C] | 142 |
| 0815 | | | |
| Cd68- | Sense | [mAs][mG][mG][mC][mG][mC][mA][fG][fA][fA][fU][mU] | 143 |
| 0815 | | [mC][mA][mU][mC][mU][mCs][mUs][mA] | |
| Cd68- | Sense | [mCs][mA][fG][fA][fA][fU][mU][mC][mA][mU][mC][mU] | 144 |
| 0815 | | [mC][mU][mA][mG][mC][mA][mG][mC][mC][mG][ademA- | |
| | | C22][mA][mA][mG][mG][mC][mU][mG][mC] | |
| Cd68- | Sense | [mCs][mA][fG][fA][fA][fU][mU][mC][mA][mU][mC][mU] | 145 |
| 0815 | | [mC][mU][mA][mG][mC][mA][mG][mC][mC][mG][ademA- | |
| | | C22][mA][mA][mG][mG][mC][mU][mG][mC] | |
| Cd68- | Sense | [mCs][mA][fG][fA][fA][fU][mU][mC][mA][mU][mC][mU] | 146 |
| 0815 | | [mC][mU][mA][mG][mC][mA][mG][mC][mC][mG][ademA- | |
| | | C22][mA][mA][mG][mG][mC][mU][mG][mC] | |
| Cd68- | Sense | [mCs][mA][fG][fA][fA][fU][mU][mC][mA][mU][mC][mU] | 147 |
| 0815 | | [mC][mU][mA][mG][mC][mA][mG][mC][mC][mG][ademA- | |
| | | C22][mA][mA][mG][mG][mC][mU][mG][mC] | |
| Cd68- | Anti- | [MePhosphonate-4O- | 148 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 149 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 150 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 151 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 152 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 153 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 154 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 155 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 156 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 157 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 158 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 159 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 160 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 161 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 162 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 163 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 164 |
| 0815 | sense | mUs][fAs][fGs][fA][fG][mA][fU][mG][mA][fA][mU][mU] | |
| | | C[m][fU][mG][mC][mG][mC][mC][mUs][mGs][mG] | |
| Cd68- | Anti- | [MePhosphonate-4O- | 165 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 166 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 167 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 168 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 169 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 170 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 171 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 172 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 173 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 174 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 175 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 176 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 177 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 178 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 179 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 180 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 181 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 182 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 183 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 184 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 185 |
| 0815 | sense | mUs][fAs][fGs][fA][fG][mA][fU][mG][mA][fA][mU][mU] | |
| | | [mCs][fUs][mG][mC][mG][mC][mC][mUs][mGs][mG] | |
| Cd68- | Anti- | [MePhosphonate-4O- | 186 |
| 0815 | sense | mUs][fAs][fGs][fA][fG][mA][fU][mG][mA][fA][mU][mU] | |
| | | [mCs][fUs][mG][mC][mG][mC][mC][mUs][mGs][mG] | |
| Cd68- | Anti- | [MePhosphonate-4O- | 187 |
| 0815 | sense | mUs][fAs][fGs][fA][fG][mA][fU][mG][mA][fA][mU][mU] | |
| | | [mCs][fUs][mG][mC][mG][mC][mC][mUs][mGs][mG] | |
| Cd68- | Anti- | [MePhosphonate-4O- | 188 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 189 |
| 0815 | sense | mUs][fAs][fGs][fA][fG][mA][fU][mG][mA][fA][mU][mU] | |
| | | [mC][fU][mG][mC][mG][mC][fC][mUs][mGs][mG] | |
| Cd68- | Anti- | [MePhosphonate-4O- | 190 |
| 0815 | sense | mUs][fAs][fGs][fA][fG][mA][fU][mG][mA][fA][mU][mU] | |
| | | [mC][fU][mG][mC][mG][mC][mC][mUs][mGs][mG] | |
| Cd68- | Anti- | [MePhosphonate-4O- | 191 |
| 0815 | sense | mUs][fAs][fGs][fA][fG][mA][fU][mG][mA][fA][mU][mU] | |
| | | [mC][fU][mG][mC][mG][mC][fCs][mUs][mGs][mG] | |
| Cd68- | Anti- | [MePhosphonate-4O- | 192 |
| 0815 | sense | mUs][fAs][fGs][fA][fG][mA][fU][mG][mA][fA][mU][mU] | |
| | | [mC][fU][mG][mC][mG][mCs][fC][mUs][mGs][mG] | |
| ALDH2- | Sense | UGAAACUCAGUUUAGCCGAAGGC | 193 |
| 1203 | | | |
| ALDH2- | Sense | TGAAACUCAGUUUAGCCGAAGGC | 194 |
| 1203 | | | |
| ALDH2- | Sense | TGAAACUCAGUUUAGCCGAAGGC | 195 |
| 1203 | | | |
| ALDH2- | Sense | TGAAACUCAGUTTAGCCGAAGGC | 196 |
| 1203 | | | |
| ALDH2- | Sense | GAAACUCAGUUUAGCCGAAGGC | 197 |
| 1203 | | | |
| ALDH2- | Sense | GAAACUCAGUUUAGCCGAAGGC | 198 |
| 1203 | | | |
| ALDH2- | Sense | GAAACUCAGUUUAGCCGAAGGC | 199 |
| 1203 | | | |
| ALDH2- | Sense | GAAACUCAGUTTAGCCGAAGGC | 200 |
| 1203 | | | |
| ALDH2- | Sense | AAACUCAGUUUAGCCGAAGGC | 201 |
| 1203 | | | |
| ALDH2- | Sense | AAACUCAGUUUAGCCGAAGGC | 202 |
| 1203 | | | |
| ALDH2- | Sense | AAACUCAGUUUAGCCGAAGGC | 203 |
| 1203 | | | |
| ALDH2- | Sense | AAACUCAGUTTAGCCGAAGGC | 204 |
| 1203 | | | |
| ALDH2- | Anti- | UAAACUGAGUUUCAUCCACCGG | 205 |
| 1203 | sense | | |
| ALDH2- | Anti- | UAAACUGAGUUUCAUCCACCGG | 206 |
| 1203 | sense | | |
| ALDH2- | Anti- | UAAACUGAGUUUCAUCCACCGG | 207 |
| 1203 | sense | | |
| ALDH2- | Anti- | UAAACUGAGUUUCAUCCACCGG | 208 |
| 1203 | sense | | |
| ALDH2- | Anti- | UAAACUGAGUUUCAUCCACCGG | 209 |
| 1203 | sense | | |
| ALDH2- | Anti- | UAAACUGAGUUUCAUCCACCGG | 210 |
| 1203 | sense | | |
| ALDH2- | Anti- | UAAACUGAGUUUCAUCCACCGG | 211 |
| 1203 | sense | | |
| ALDH2- | Anti- | UAAACUGAGUUUCAUCCACCGG | 212 |
| 1203 | sense | | |
| ALDH2- | Anti- | UAAACUGAGUUUCAUCCACCGG | 213 |
| 1203 | sense | | |
| ALDH2- | Anti- | UAAACUGAGUUUCAUCCACCGG | 214 |
| 1203 | sense | | |
| ALDH2- | Anti- | UAAACUGAGUUUCAUCCACCGG | 215 |
| 1203 | sense | | |
| ALDH2- | Anti- | UAAACUGAGUUUCAUCCACCGG | 216 |
| 1203 | sense | | |
| ALDH2- | Sense | [mUs][fG][[A][fA][fA][mC][mU][mC][mA][mG][mU][mU] | 217 |
| 1203 | | [mU][mA][+G][+C][+C][mG][ademA-GalNAc][ademA- | |
| | | GalNAc][+G][+G][+C] | |
| ALDH2- | Sense | [+Ts][fG][fA][fA][fA][mC][mU][mC][+A][mG][mU][mU] | 218 |
| 1203 | | [mU][mA][+G][+C][+C][mG][ademA-GalNAc][ademA- | |
| | | GalNAc][+G][+G][+C] | |
| ALDH2- | Sense | [+Ts][fG][fA][fA][fA][mC][mU][mC][+A][+G][mU][mU] | 219 |
| 1203 | | [mU][mA][+G][+C][+C][mG][ademA-GalNAc][ademA- | |
| | | GalNAc][+G][+G][+C] | |
| ALDH2- | Sense | [+Ts][fG][fA][fA][fA][mC][mU][mC][+A][+G][mU][+T][+T] | 220 |
| 1203 | | [mA][+G][+C][+C][mG][ademA-GalNAc][ademA- | |
| | | GalNAc][+G][+G][+C] | |
| ALDH2- | Sense | [fGs][fA][fA][fA][mC][mU][mC][mA][mG][mU][mU][mU] | 221 |
| 1203 | | [mA][+G][+C][+C][mG][ademA-GalNAc][ademA- | |
| | | GalNAc][+G][+G][+C] | |
| ALDH2- | Sense | [+Gs][fA][fA][fA][mC][mU][mC][+A][mG][mU][mU][mU] | 222 |
| 1203 | | [mA][+G][+C][+C][mG][ademA-GalNAc][ademA- | |
| | | GalNAc][+G][+G][+C] | |
| ALDH2- | Sense | [+Gs][fA][fA][fA][mC][mU][mC][+A][+G][mU][mU][mU] | 223 |
| 1203 | | [mA][+G][+C][+C][mG][ademA-GalNAc][ademA- | |
| | | GalNAc][+G][+G][+C] | |
| ALDH2- | Sense | [+Gs][[A][[A][fA][mC][mU][mC][+A][+G][mU][+T][+T] | 224 |
| 1203 | | [mA][+G][+C][+C][mG][ademA-GalNAc][ademA- | |
| | | GalNAc][+G][+G][+C] | |
| ALDH2- | Sense | [fAs][fA][fA][mC][mU][mC][mA][mG][mU][mU][mU][mA] | 225 |
| 1203 | | [+G][+C][+C][mG][ademA-GalNAc][ademA- | |
| | | GalNAc][+G][+G][+C] | |
| ALDH2- | Sense | [+As][fA][fA][mC][mU][mC][+A][mG][mU][mU][mU][mA] | 226 |
| 1203 | | [+G][+C][+C][mG][ademA-GalNAc][ademA- | |
| | | GalNAc][+G][+G][+C] | |
| ALDH2- | Sense | [+As][fA][fA][mC][mU][mC][+A][+G][mU][mU][mU][mA] | 227 |
| 1203 | | [+G][+C][+C][mG][ademA-GalNAc][ademA- | |
| | | GalNAc][+G][+G][+C] | |
| ALDH2- | Sense | [+As][fA][fA][mC][mU][mC][+A][+G][mU][+T][+]][mA] | 228 |
| 1203 | | [+G][+C][+C][mG][ademA-GalNAc][ademA- | |
| | | GalNAc][+G][+G][+C] | |
| ALDH2- | Anti- | [MePhosphonate-4O- | 229 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 230 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 231 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 232 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 233 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 234 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 235 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 236 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 237 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 238 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 239 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Anti- | [MePhosphonate-4O- | 240 |
| 1203 | sense | mUs][fAs][fA][fA][fC][mU][fG][mA][mG][fU][mU][mU] | |
| | | [mCs][fAs][mU][mC][mC][mA][mC][mCs][mGs][mG] | |
| ALDH2- | Sense | GAUGAAACUCAGUUUA | 241 |
| 1203 | | | |
| ALDH2- | Sense | GAUGAAACUCAGUUUA | 242 |
| 1203 | | | |
| ALDH2- | Sense | GAUGAAACUCAGUUUA | 243 |
| 1203 | | | |
| ALDH2- | Sense | GAUGAAACUCAGUUUA | 244 |
| 1203 | | | |
| ALDH2- | Sense | GAUGAAACUCAGUUUA | 245 |
| 1203 | | | |
| ALDH2- | Sense | GAUGAAACUCAGUUUA | 246 |
| 1203 | | | |
| ALDH2- | Sense | GAUGAAACUCAGUUUA | 247 |
| 1203 | | | |
| ALDH2- | Sense | GAUGAAACUCAGUUUA | 248 |
| 1203 | | | |
| ALDH2- | Sense | GAUGAAACUCAGUUUA | 249 |
| 1203 | | | |
| ALDH2- | Anti- | UAAACUGAGUUUCAUCCACUGG | 250 |
| 1203 | sense | | |
| ALDH2- | Anti- | UAAACUGAGUUUCAUCCACUGG | 251 |
| 1203 | sense | | |
| ALDH2- | Anti- | UAAACUGAGUUUCAUCCACUGG | 252 |
| 1203 | sense | | |
| ALDH2- | Anti- | UAAACUGAGUUUCAUCCACUGG | 253 |
| 1203 | sense | | |
| ALDH2- | Anti- | UAAACUGAGUUUCAUCCACUGG | 254 |
| 1203 | sense | | |
| ALDH2- | Anti- | UAAACUGAGUUUCAUCCACUGG | 255 |
| 1203 | sense | | |
| ALDH2- | Anti- | UAAACUGAGUUUCAUCCACUGG | 256 |
| 1203 | sense | | |
| ALDH2- | Anti- | UAAACUGAGUUUCAUCCACUGG | 257 |
| 1203 | sense | | |
| ALDH2- | Anti- | UAAACUGAGUUUCAUCCACUGG | 258 |
| 1203 | sense | | |
| ALDH2- | Anti- | UAAACUGAGUUUCAUCCACUGG | 259 |
| 1203 | sense | | |
| ALDH2- | Sense | [+Gs][mA][mU][fG][fA][fA][fA][mC][mU][mC][mA][mG] | 260 |
| 1203 | | [mU][mUs][mUs][ademA-C16] | |
| ALDH2- | Sense | [+Gs][mA][mU][fG][fA][fA][fA][mC][mU][mC][+A][mG] | 261 |
| 1203 | | [mU][mUs][mUs][ademA-C16] | |
| ALDH2- | Sense | [+Gs][mA][mU][fG][fA][fA][fA][mC][mU][mC][+A][+G] | 262 |
| 1203 | | [mU][mUs][mUs][ademA-C16] | |
| ALDH2- | Sense | [ademGs- | 263 |
| 1203 | | C16][+A][mU][fG][fA][fA][fA][mC][mU][mC][mA][mG] | |
| | | [mU][mUs][mUs][mA] | |
| ALDH2- | Sense | [ademGs-C16][+A][mU][fG][fA][fA][fA][mC] | 264 |
| 1203 | | [mU][mC][+A][mG][mU][mUs][mUs][mA] | |
| ALDH2- | Sense | [ademGs-C16][+A][mU][fG][fA][fA][fA][mC][mU] | 265 |
| 1203 | | [mC][+A][+G][mU][mUs][mUs][mA] | |
| ALDH2- | Sense | [+Gs][mA][mU][fG][ademA-C16][fA][fA][mC] | 266 |
| 1203 | | [mU][mC][mA][mG][mU][mUs][mUs][mA] | |
| ALDH2- | Sense | [+Gs][mA][mU][fG][ademA-C16][fA][fA][mC][mU][mC] | 267 |
| 1203 | | [+A][mG][mU][mUs][mUs][mA] | |
| ALDH2- | Sense | [+Gs][mA][mU][fG][ademA-C16][fA][fA][mC][mU][mC] | 268 |
| 1203 | | [+A][+G][mU][mUs][mUs][mA] | |
| ALDH2- | Anti- | [MePhosphonate-4O- | 269 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 270 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 271 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 272 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 273 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 274 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 275 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 276 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| ALDH2- | Anti- | [MePhosphonate-4O- | 277 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| STAT3- | Sense | AGUUGAAUUAUCAGCUUAAA | 278 |
| 1402 | | | |
| SLC25A1- | Sense | ACCAUCAAGGUGAAGUUCAA | 279 |
| 0597 | | | |
| HMGB1- | Sense | AGAAAAAAAUUGAAAUGUAA | 280 |
| 0932 | | | |
| ALDH2- | Sense | AGUGGAUGAAACUCAGUUUA | 281 |
| 1203 | | | |
| STAT3- | Sense | AGUUGAAUUAUCAGCUUA | 282 |
| 1402 | | | |
| SLC25A1- | Sense | ACCAUCAAGGUGAAGUUC | 283 |
| 0597 | | | |
| HMGB1- | Sense | AGAAAAAAAUUGAAAUGU | 284 |
| 0932 | | | |
| ALDH2- | Sense | AGUGGAUGAAACUCAGUU | 285 |
| 1203 | | | |
| STAT3- | Anti- | UUUAAGCUGAUAAUUCAACUGG | 286 |
| 1402 | sense | | |
| SLC25A1- | Anti- | UUGAACUUCACCUUGAUGGUGG | 287 |
| 0597 | sense | | |
| HMGB1- | Anti- | UUACAUUUCAAUUUUUUUCUGG | 288 |
| 0932 | sense | | |
| ALDH2- | Anti- | UAAACUGAGUUUCAUCCACUGG | 289 |
| 1203 | sense | | |
| STAT3- | Anti- | UUUAAGCUGAUAAUUCAACUGG | 290 |
| 1402 | sense | | |
| SLC25A1- | Anti- | UUGAACUUCACCUUGAUGGUGG | 291 |
| 0597 | sense | | |
| HMGB1- | Anti- | UUACAUUUCAAUUUUUUUCUGG | 292 |
| 0932 | sense | | |
| ALDH2- | Anti- | UAAACUGAGUUUCAUCCACUGG | 293 |
| 1203 | sense | | |
| STAT3- | Sense | [ademAs-C22][mG][mU][mU][mG][mA][mA][fU][fU] | 294 |
| 1402 | | [fA][fU][mC][mA][mG][mC][mU][mU][mAs][mAs][mA] | |
| SLC25A1- | Sense | [ademAs-C22][mC][mC][mA][mU][mC][mA][fA][fG] | 295 |
| 0597 | | [fG][fU][mG][mA][mA][mG][mU][mU][mCs][mAs][mA] | |
| HMGB1- | Sense | [ademAs- | 296 |
| 0932 | | C22][mG][mA][mA][mA][mA][mA][fA][fA][fU][fU][mG] | |
| | | [mA][mA][mA][mU][mG][mUs][mAs][mA] | |
| ALDH2- | Sense | [ademAs-C22][mG][mU][mG][mG][mA][mU][fG][fA] | 297 |
| 1203 | | [fA][fA][mC][mU][mC][mA][mG][mU][mUs][mUs][mA] | |
| STAT3- | Sense | [ademAs-C22][mG][mU][mU][mG][mA][mA][fU][fU] | 298 |
| 1402 | | [[A][fU][mC][mA][mG][mC][mUs][mUs][mA] | |
| SLC25A1- | Sense | [ademAs-C22][mC][mC][mA][mU][mC][mA][fA][fG] | 299 |
| 0597 | | [fG][fU][mG][mA][mA][mG][mUs][mUs][mC] | |
| HMGB1- | Sense | [adem As-C22][mG][mA][mA][mA][mA][mA][fA] | 300 |
| 0932 | | [[A][fU][fU][mG][mA][mA][mA][mUs][mGs][mU] | |
| ALDH2- | Sense | [ademAs-C22][mG][mU][mG][mG][mA][mU][fG] | 301 |
| 1203 | | [fA][fA][fA][mC][mU][mC][mA][mGs][mUs][mU] | |
| STAT3- | Anti- | [MePhosphonate-4O-mUs][fUs][fUs][fA][fA][mG][fC][mU] | 302 |
| 1402 | sense | [mG][fA][mU][mA][mA][fU][mU][mC][mA][mA][mC][mUs | |
| | | ][mGs][mG] | |
| SLC25A1- | Anti- | [MePhosphonate-4O-mUs][fUs][fGs][fA][fA][mC][fU] | 303 |
| 0597 | sense | [mU][mC][fA][mC][mC][mU][fU][mG][mA][mU][mG][mG] | |
| | | [mUs][mGs][mG] | |
| HMGB1- | Anti- | [MePhosphonate-4O- | 304 |
| 0932 | sense | mUs][fUs][fAs][fC][fA][mU][fU][mU][mC][fA][mA][mU] | |
| | | [mU][fU][mU][mU][mU][mU][mC][mUs][mGs][mG] | |
| ALDH2- | Anti- | [MePhosphonate-4O- | 305 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| STAT3- | Anti- | [MePhosphonate-4O- | 306 |
| 1402 | sense | mUs][fUs][fUs][fA][fA][mG][fC][mU][mG][fA][mU][mA] | |
| | | [mA][fU][mU][mC][mA][mA][mC][mUs][mGs][mG] | |
| SLC25A1- | Anti- | [MePhosphonate-4O- | 307 |
| 0597 | sense | mUs][fUs][fGs][fA][fA][mC][fU][mU][mC][fA][mC][mC] | |
| | | [mU][fU][mG][mA][mU][mG][mG][mUs][mGs][mG] | |
| HMGB1- | Anti- | [MePhosphonate-4O- | 308 |
| 0932 | sense | mUs][fUs][fAs][fC][fA][mU][fU][mU][mC][fA][mA][mU] | |
| | | [mU][fU][mU][mU][mU][mU][mC][mUs][mGs][mG] | |
| ALDH2- | Anti- | [MePhosphonate-4O- | 309 |
| 1203 | sense | mUs][fAs][fAs][fA][fC][mU][fG][mA][mG][fU][mU][mU] | |
| | | [mC][fA][mU][mC][mC][mA][mC][mUs][mGs][mG] | |
| TUBB3- | Sense | AGUGUGAGAAUUGUGACUGAGCAGCCGAAAGGCUG | 310 |
| 0445 | | C | |
| TUBB3- | Sense | GCAGCCGAAAGGCUGCCCAGUGUGAGAAUUGUGAC | 311 |
| 0445 | | UGA | |
| TUBB3- | Sense | GCACGAAAGUGCCCAGUGUGAGAAUUGUGACUGA | 312 |
| 0445 | | | |
| TUBB3- | Sense | GCACGAAAGUGCCCAGUGUGAGAAUUGUGACUGA | 313 |
| 0445 | | | |
| TUBB3- | Sense | GCACGAAAGUGCCCAGUGUGAGAAUUGUGACUGA | 314 |
| 0445 | | | |
| TUBB3- | Sense | GCACUACGGUGCCCAGUGUGAGAAUUGUGACUGA | 315 |
| 0445 | | | |
| TUBB3- | Sense | GCUACGGCCCAGUGUGAGAAUUGUGACUGA | 316 |
| 0445 | | | |
| TUBB3- | Sense | GCUACGGCCCAGUGUGAGAAUUGUGACUGA | 317 |
| 0445 | | | |
| TUBB3- | Sense | GCUACGGCCCAGUGUGAGAAUUGUGACU | 318 |
| 0445 | | | |
| TUBB3- | Sense | GCAGCCGAAAGGCUGCCCAGUGUGAGAAUUGUGAC | 319 |
| 0445 | | UGA | |
| TUBB3- | Sense | GCACGAAAGUGCCCAGUGUGAGAAUUGUGACUGA | 320 |
| 0445 | | | |
| TUBB3- | Sense | GCACGAAAGUGCCCAGUGUGAGAAUUGUGACUGA | 321 |
| 0445 | | | |
| TUBB3- | Sense | GCACGAAAGTGCCCAGUGUGAGAAUUGUGACUGA | 322 |
| 0445 | | | |
| TUBB3- | Sense | GCUACGGCCCAGUGUGAGAAUUGUGACUGA | 323 |
| 0445 | | | |
| TUBB3- | Anti- | UCAGUCACAAUUCUCACACUGG | 324 |
| 0445 | sense | | |
| TUBB3- | Anti- | UCAGUCACAAUUCUCACACUGG | 325 |
| 0445 | sense | | |
| TUBB3- | Anti- | UCAGUCACAAUUCUCACACUGG | 326 |
| 0445 | sense | | |
| TUBB3- | Anti- | UCAGUCACAAUUCUCACACUGG | 327 |
| 0445 | sense | | |
| TUBB3- | Anti- | UCAGUCACAAUUCUCACACUGG | 328 |
| 0445 | sense | | |
| TUBB3- | Anti- | UCAGUCACAAUUCUCACACUGG | 329 |
| 0445 | sense | | |
| TUBB3- | Anti- | UCAGUCACAAUUCUCACACUGG | 330 |
| 0445 | sense | | |
| TUBB3- | Anti- | UCAGUCACAAUUCUCACACUGG | 331 |
| 0445 | sense | | |
| TUBB3- | Anti- | UCAGUCACAAUUCUCACACUGG | 332 |
| 0445 | sense | | |
| TUBB3- | Anti- | UCAGUCACAAUUCUCACACUGG | 333 |
| 0445 | sense | | |
| TUBB3- | Anti- | UCAGUCACAAUUCUCACACUGG | 334 |
| 0445 | sense | | |
| TUBB3- | Anti- | UCAGUCACAAUUCUCACACUGG | 335 |
| 0445 | sense | | |
| TUBB3- | Anti- | UCAGUCACAAUUCUCACACUGG | 336 |
| 0445 | sense | | |
| TUBB3- | Anti- | UCAGUCACAAUUCUCACACUGG | 337 |
| 0445 | sense | | |
| TUBB3- | Sense | [mAs][mG][mU][mG][mU][mG][mA][fG][fA][fA][fU][mU] | 338 |
| 0445 | | [mG][mU][mG][mA][mC][mU][mG][mA][mG][mC][mA][mG] | |
| | | [mC][mC][mG][ademA- | |
| | | C16][mA][mA][mG][mG][mC][mU][mG][mC] | |
| TUBB3- | Sense | [mG][mC][mA][mG][mC][mC][mG][ademA- | 339 |
| 0445 | | C16][mA][mA][mG][mG][mC][mU][mG][mC][mC][mC][mA] | |
| | | [mG][mU][mG][mU][mG][mA][fG][fA][fA][fU][mU][mG] | |
| | | [mU][mG][mA][mC][mUs][mGs][mA] | |
| TUBB3- | Sense | [mG][mC][mA][mC][mG][ademA- | 340 |
| 0445 | | C16][mA][mA][mG][mU][mG][mC][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [mGs][mCs][mA][mC][mG][ademA- | 341 |
| 0445 | | C16][mA][mA][mG][mU][mG][mC][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [mG][mC][mA][mC][mG][mA][mA][mA][mG][mU][mG] | 342 |
| 0445 | | [mC][mC][mC][ademA- | |
| | | C16][mG][mU][mG][mU][mG][mA][fG][fA][fA][fU][mU] | |
| | | [mG][mU][mG][mA][mC][mUs][mGs][mA] | |
| TUBB3- | Sense | [mG][mC][mA][mC][mU][ademA- | 343 |
| 0445 | | C16][mC][mG][mG][mU][mG][mC][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [mG][mC][mU][ademA- | 344 |
| 0445 | | C16][mC][mG][mG][mC][mC][mC][mA][mG][mU][mG][mU] | |
| | | [mG][mA][fG][fA][fA][fU][mU][mG][mU][mG][mA][mC] | |
| | | [mUs][mGs][mA] | |
| TUBB3- | Sense | [+G][mC][mU][ademA- | 345 |
| 0445 | | C16][mC][mG][mG][+C][mC][mC][mA][mG][mU][mG][mU] | |
| | | [mG][mA][fG][fA][fA][fU][mU][mG][mU][mG][mA][mC] | |
| | | [mUs][mGs][mA] | |
| TUBB3- | Sense | [+G][mC][mU][ademA- | 346 |
| 0445 | | C16][mC][mG][mG][+C][mC][mC][mA][mG][mU][mG][mU] | |
| | | [mG][mA][fG][fA][fA][fU][mU][mG][mU][mG][mAs][mCs] | |
| | | [mU] | |
| TUBB3- | Sense | [mG][mC][mA][mG][mC][mC][mG][mA][mA][mA][mG][mG] | 347 |
| 0445 | | [mC][mU][mG][mC][mC][mC][mA][mG][mU][mG][mU] | |
| | | [mG][mA][fG][fA][fA][fU][mU][mG][mU][mG][mA][mC] | |
| | | [mUs][mGs][ademA-C16] | |
| TUBB3- | Sense | [+G][mC][mA][mC][mG][ademA- | 348 |
| 0445 | | C16][mA][mA][mG][mU][mG][+C][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [+G][+C][mA][mC][mG][ademA- | 349 |
| 0445 | | C16][mA][mA][mG][mU][+G][+C][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [+G][+C][+A][mC][mG][ademA- | 350 |
| 0445 | | C16][mA][mA][mG][+T][+G][+C][mC][mC][mA][mG][mU] | |
| | | [mG][mU][mG][mA][fG][fA][fA][fU][mU][mG][mU][mG] | |
| | | [mA][mC][mUs][mGs][mA] | |
| TUBB3- | Sense | [+G][+C][mU][ademA- | 351 |
| 0445 | | C16][mC][mG][+G][+C][mC][mC][mA][mG][mU][mG][mU] | |
| | | [mG][mA][fG][fA][fA][fU][mU][mG][mU][mG][mA][mC] | |
| | | [mUs][mGs][mA] | |
| TUBB3- | Anti- | [MePhosphonate-4O- | 352 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 353 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 354 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 355 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 356 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 357 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 358 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 359 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 360 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 361 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 362 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 363 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 364 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 365 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Sense | GCUACGGCCCAGUGUGAGAAUUGUGACUGA | 366 |
| 0445 | | | |
| TUBB3- | Sense | GCUACGGCCCAGUGUGAGAAUUGUGACUGA | 367 |
| 0445 | | | |
| TUBB3- | Sense | GCUACGGCCCAGUGUGAGAAUUGUGACU | 368 |
| 0445 | | | |
| TUBB3- | Sense | GCUACGGCCCAGUGUGAGAAUUGUGACU | 369 |
| 0445 | | | |
| TUBB3- | Sense | AGUGUGAGAAUUGUGACU | 370 |
| 0445 | | | |
| TUBB3- | Sense | AGUGUGAGAAUUGUGACU | 371 |
| 0445 | | | |
| TUBB3- | Sense | AGUGUGAGAAUUGUGACUGA | 372 |
| 0445 | | | |
| TUBB3- | Anti- | UCAGUCACAAUUCUCACACUGG | 373 |
| 0445 | sense | | |
| TUBB3- | Anti- | UCAGUCACAAUUCUCACACUGG | 374 |
| 0445 | sense | | |
| TUBB3- | Anti- | UCAGUCACAAUUCUCACACUGG | 375 |
| 0445 | sense | | |
| TUBB3- | Anti- | AGUCACAAUUCUCACACUGG | 376 |
| 0445 | sense | | |
| TUBB3- | Anti- | UCAGUCACAAUUCUCACACUGG | 377 |
| 0445 | sense | | |
| TUBB3- | Anti- | AGUCACAAUUCUCACACUGG | 378 |
| 0445 | sense | | |
| TUBB3- | Anti- | UCAGUCACAAUUCUCACACUGG | 379 |
| 0445 | sense | | |
| TUBB3- | Sense | [mG][mC][mU][ademA- | 380 |
| 0445 | | C16][mC][mG][mG][mC][mC][mC][mA][mG][mU][mG][mU] | |
| | | [mG][mA][fG][fA][fA][fU][mU][mG][mU][mG][mA][mC] | |
| | | [mUs][mGs][mA] | |
| TUBB3- | Sense | [+G][mC][mU][ademA-C16][mC][mG][mG][+C][mC] | 381 |
| 0445 | | [mC][mA][mG][mU][mG][mU][mG][mA][fG][fA][fA][fU] | |
| | | [mU][mG][mU][mG][mA][mC][mUs][mGs][mA] | |
| TUBB3- | Sense | [+G][mC][mU][ademA-C16][mC][mG][mG][+C] | 382 |
| 0445 | | [mC][mC][mA][mG][mU][mG][mU][mG][mA][fG][fA][fA] | |
| | | [fU][mU][mG][mU][mG][mAs][mCs][mU] | |
| TUBB3- | Sense | [+G][mC][mU][ademA-C16][mC][mG][mG][+C] | 383 |
| 0445 | | [mC][mC][mA][mG][mU][mG][mU][mG][mA][fG][fA][fA] | |
| | | [fU][mU][mG][mU][mG][mAs][mCs][mU] | |
| TUBB3- | Sense | [ademAs-C16][mG][mU][mG][mU][mG][mA][fG] | 384 |
| 0445 | | [fA][fA][fU][mU][mG][mU][mG][mAs][mCs][mU] | |
| TUBB3- | Sense | [ademAs-C16][mG][mU][mG][mU][mG][mA][fG] | 385 |
| 0445 | | [fA][fA][fU][mU][mG][mU][mG][mAs][mCs][mU] | |
| TUBB3- | Sense | [ademAs-C16][mG][mU][mG][mU][mG][mA][fG][fA] | 386 |
| 0445 | | [fA][fU][mU][mG][mU][mG][mA][mC][mUs][mGs][mA] | |
| TUBB3- | Anti- | [MePhosphonate-4O- | 387 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 388 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 389 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [fAs][fG][fU][mC][fA][mC][mA][fA][mU][mU][mC][fU][mC] | 390 |
| 0445 | sense | [mA][mC][mA][mC][mUs][mGs][mG] | |
| TUBB3- | Anti- | [MePhosphonate-4O- | 391 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [fAs][fG][fU][mC][fA][mC][mA][fA][mU][mU][mC][fU][mC] | 392 |
| 0445 | sense | [mA][mC][mA][mC][mUs][mGs][mG] | |
| TUBB3- | Anti- | [MePhosphonate-4O- | 393 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Sense | AGUGUGAGAAUUGUGACUGA | 394 |
| 0445 | | | |
| TUBB3- | Sense | AGUGUGAGAAUUGUGACUG | 395 |
| 0445 | | | |
| TUBB3- | Sense | AGUGUGAGAAUUGUGACU | 396 |
| 0445 | | | |
| TUBB3- | Sense | AGUGUGAGAAUUGUGAC | 397 |
| 0445 | | | |
| TUBB3- | Sense | AGUGUGAGAAUUGUGA | 398 |
| 0445 | | | |
| TUBB3- | Sense | AGUGUGAGAAUUGUG | 399 |
| 0445 | | | |
| TUBB3- | Sense | AGUGUGAGAAUUGU | 400 |
| 0445 | | | |
| TUBB3- | Sense | AGUGUGAGAAUUG | 401 |
| 0445 | | | |
| TUBB3- | Sense | AGUGUGAGAAUU | 402 |
| 0445 | | | |
| TUBB3- | Sense | AGUGUGAGAAUUGUGA | 403 |
| 0445 | | | |
| TUBB3- | Sense | AGUGUGAGAAUUGUG | 404 |
| 0445 | | | |
| TUBB3- | Anti- | UCAGUCACAAUUCUCACACUGG | 405 |
| 0445 | sense | | |
| TUBB3- | Anti- | UCAGUCACAAUUCUCACACUGG | 406 |
| 0445 | sense | | |
| TUBB3- | Anti- | UCAGUCACAAUUCUCACACUGG | 407 |
| 0445 | sense | | |
| TUBB3- | Anti- | UCAGUCACAAUUCUCACACUGG | 408 |
| 0445 | sense | | |
| TUBB3- | Anti- | UCAGUCACAAUUCUCACACUGG | 409 |
| 0445 | sense | | |
| TUBB3- | Anti- | UCAGUCACAAUUCUCACACUGG | 410 |
| 0445 | sense | | |
| TUBB3- | Anti- | UCAGUCACAAUUCUCACACUGG | 411 |
| 0445 | sense | | |
| TUBB3- | Anti- | UCAGUCACAAUUCUCACACUGG | 412 |
| 0445 | sense | | |
| TUBB3- | Anti- | UCAGUCACAAUUCUCACACUGG | 413 |
| 0445 | sense | | |
| TUBB3- | Anti- | UCAGUCACAAUUCUCACACUGG | 414 |
| 0445 | sense | | |
| TUBB3- | Anti- | UCAGUCACAAUUCUCACACUGG | 415 |
| 0445 | sense | | |
| TUBB3- | Sense | [ademAs-C16][mG][mU][mG][mU][mG][mA][fG][fA] | 416 |
| 0445 | | [fA][fU][mU][mG][mU][mG][mA][mC][mUs][mGs][mA] | |
| TUBB3- | Sense | [ademAs-C16][mG][mU][mG][mU][mG][mA][fG][A] | 417 |
| 0445 | | [fA][fU][mU][mG][mU][mG][mA][mCs][mUs][mG] | |
| TUBB3- | Sense | [ademAs-C16][mG][mU][mG][mU][mG][mA][fG][fA] | 418 |
| 0445 | | [fA][fU][mU][mG][mU][mG][mAs][mCs][mU] | |
| TUBB3- | Sense | [ademAs-C16][mG][mU][mG][mU][mG][mA][fG] | 419 |
| 0445 | | [fA][fA][fU][mU][mG][mU][mGs][mAs][mC] | |
| TUBB3- | Sense | [ademAs-C16][mG][mU][mG][mU][mG][mA][fG] | 420 |
| 0445 | | [[A][fA][fU][mU][mG][mUs][mGs][mA] | |
| TUBB3- | Sense | [ademAs-C16][mG][mU][mG][mU][mG][mA][fG][fA] | 421 |
| 0445 | | [fA][fU][mU][mGs][mUs][mG] | |
| TUBB3- | Sense | [ademAs-C16][mG][mU][mG][mU][mG][mA][fG][fA] | 422 |
| 0445 | | [fA][fU][mUs][mGs][mU] | |
| TUBB3- | Sense | [ademAs-C16][mG][mU][mG][mU][mG][mA][fG][fA] | 423 |
| 0445 | | [fA][fUs][mUs][mG] | |
| TUBB3- | Sense | [ademAs-C16][mG][mU][mG][mU][mG][mA][G][fA] | 424 |
| 0445 | | [fAs][fUs][mU] | |
| TUBB3- | Sense | [ademAs-C16][mG][mU][mG][mU][mG][mA][fG][fA] | 425 |
| 0445 | | [fA][fU][mU][mG][mUs][mGs][mA] | |
| TUBB3- | Sense | [ademAs-C16][mG][mU][mG][mU][mG][mA][fG][fA] | 426 |
| 0445 | | [fA][fU][mU][mGs][mUs][mG] | |
| TUBB3- | Anti- | [MePhosphonate-4O- | 427 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 428 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 429 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 430 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 431 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 432 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 433 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 434 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 435 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 436 |
| 0445 | sense | mUs][fCs][fAs][fGs][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 437 |
| 0445 | sense | mUs][fCs][fAs][fGs][fUs][mC][fA][mC][mA][fA][mU][mU] | |
| | | [mC][fU][mC][mA][mC][mA][mC][mUs][mGs][mG] | |
| TUBB3- | Sense | AGUGUGAGAAUUGUGACUGA | 438 |
| 0445 | | | |
| TUBB3- | Sense | AGUGUGAGAAUUGUGACTGA | 439 |
| 0445 | | | |
| TUBB3- | Sense | AGUGUGAGAAUUGUGACTG | 440 |
| 0445 | | | |
| TUBB3- | Sense | AGUGUGAGAAUUGUGACT | 441 |
| 0445 | | | |
| TUBB3- | Sense | AGUGUGAGAAUUGUGAC | 442 |
| 0445 | | | |
| TUBB3- | Sense | AGUGUGAGAAUUGUGA | 443 |
| 0445 | | | |
| TUBB3- | Sense | AGUGUGAGAAUUGUG | 444 |
| 0445 | | | |
| TUBB3- | Sense | AGUGUGAGAAUUGU | 445 |
| 0445 | | | |
| TUBB3- | Sense | AGUGUGAGAAUUG | 446 |
| 0445 | | | |
| TUBB3- | Sense | AGUGUGAGAAUU | 447 |
| 0445 | | | |
| TUBB3- | Sense | AGUGUGAGAAUUGUGA | 448 |
| 0445 | | | |
| TUBB3- | Anti- | UCAGUCACAAUUCUCACACUGG | 449 |
| 0445 | sense | | |
| TUBB3- | Anti- | UCAGUCACAAUUCUCACACUGG | 450 |
| 0445 | sense | | |
| TUBB3- | Anti- | UCAGUCACAAUUCUCACACUGG | 451 |
| 0445 | sense | | |
| TUBB3- | Anti- | UCAGUCACAAUUCUCACACUGG | 452 |
| 0445 | sense | | |
| TUBB3- | Anti- | UCAGUCACAAUUCUCACACUGG | 453 |
| 0445 | sense | | |
| TUBB3- | Anti- | UCAGUCACAAUUCUCACACUGG | 454 |
| 0445 | sense | | |
| TUBB3- | Anti- | UCAGUCACAAUUCUCACACUGG | 455 |
| 0445 | sense | | |
| TUBB3- | Anti- | UCAGUCACAAUUCUCACACUGG | 456 |
| 0445 | sense | | |
| TUBB3- | Anti- | UCAGUCACAAUUCUCACACUGG | 457 |
| 0445 | sense | | |
| TUBB3- | Anti- | UCAGUCACAAUUCUCACACUGG | 458 |
| 0445 | sense | | |
| TUBB3- | Anti- | UCAGUCACAAUUCUCACACUGG | 459 |
| 0445 | sense | | |
| TUBB3- | Sense | [ademAs-C16][mG][mU][mG][mU][mG][mA][fG][fA] | 460 |
| 0445 | | [fA][fU][mU][mG][mU][mG][mA][mC][mUs][mGs][mA] | |
| TUBB3- | Sense | [ademAs-C16][+G][mU][mG][mU][mG][mA][fG] | 46 |
| 0445 | | [fA][fA][fU][mU][mG][mU][+G][+A][mC][+Ts][+Gs][mA] | |
| TUBB3- | Sense | [ademAs-C16][+G][mU][mG][mU][mG][mA][fG] | 462 |
| 0445 | | [fA][fA][fU][mU][mG][mU][+G][+A][mCs][+Ts][+G] | |
| TUBB3- | Sense | [ademAs-C16][+G][mU][mG][mU][mG][mA][fG][fA] | 463 |
| 0445 | | [fA][fU][mU][mG][mU][+G][+As][mCs][+T] | |
| TUBB3- | Sense | [ademAs-C16][+G][mU][mG][mU][mG][mA][fG][fA] | 464 |
| 0445 | | [fA][fU][mU][mG][mU][+Gs][+As][mC] | |
| TUBB3- | Sense | [ademAs-C16][+G][mU][mG][mU][mG][mA][fG][fA] | 465 |
| 0445 | | [fA][fU][mU][mG][mUs][+Gs][+A] | |
| TUBB3- | Sense | [adem As-C16][+G][mU][mG][mU][mG][mA][fG][fA] | 466 |
| 0445 | | [fA][fU][mU][mGs][mUs][+G] | |
| TUBB3- | Sense | [ademAs-C16][+G][mU][mG][mU][mG][mA][fG][fA] | 467 |
| 0445 | | [fA][fU][mUs][mGs][mU] | |
| TUBB3- | Sense | [ademAs-C16][+G][mU][mG][mU][mG][mA][fG][fA] | 468 |
| 0445 | | [fA][fUs][mUs][mG] | |
| TUBB3- | Sense | [ademAs-C16][+G][mU][mG][mU][mG][mA][fG][fA] | 469 |
| 0445 | | [fAs][fUs][mU] | |
| TUBB3- | Sense | [ademAs-C16][+G][mU][mG][mU][mG][mA][fG][A] | 470 |
| 0445 | | [fA][fU][mU][mG][mUs][+Gs][+A] | |
| TUBB3- | Anti- | [MePhosphonate-4O- | 471 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 472 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 473 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 474 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 475 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 476 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 477 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 478 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 479 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 480 |
| 0445 | sense | mUs][fCs][fAs][fG][fU][mC][fA][mC][mA][fA][mU][mU] | |
| | | [mC][fU][mC][mA][mC][mA][mC][mUs][mGs][mG] | |
| TUBB3- | Anti- | [MePhosphonate-4O- | 481 |
| 0445 | sense | mUs][fCs][fAs][fGs][fU][mC][fA][mC][mA][fA][mU][mU] | |
| | | [mC][fU][mC][mA][mC][mA][mC][mUs][mGs][mG] | |
| RTN4- | Sense | AGUUGACUUAUUUAGUGAUA | 482 |
| 2481 | | | |
| RTN4- | Sense | ACUUAUUUAGUGAUA | 483 |
| 2481 | | | |
| RTN4- | Sense | AGUUGACUUAUUUAGUGA | 484 |
| 2481 | | | |
| RTN4- | Sense | ACUUAUUUAGUGA | 485 |
| 2481 | | | |
| RTN4- | Sense | AGUUGACUUAUUUAGUGA | 486 |
| 2481 | | | |
| RTN4- | Sense | AGUUGACUUAUUUAGUGA | 487 |
| 2481 | | | |
| RTN4- | Sense | ACUUAUUUAGUGAUA | 488 |
| 2481 | | | |
| RTN4- | Sense | GCUACGGCCCAGUUGACUUAUUUAGUGA | 489 |
| 2481 | | | |
| RTN4- | Sense | GCUACGGCCCAGUUGACUUAUUUAGUGA | 490 |
| 2481 | | | |
| RTN4- | Sense | AGUUGACUUAUUUAGTGAUA | 491 |
| 2481 | | | |
| RTN4- | Sense | ACUUAUUUAGTGAUA | 492 |
| 2481 | | | |
| RTN4- | Anti- | UAUCACUAAAUAAGUCAACUGG | 493 |
| 2481 | Sense | | |
| RTN4- | Anti- | UAUCACUAAAUAAGUCAACUGG | 494 |
| 2481 | Sense | | |
| RTN4- | Anti- | UAUCACUAAAUAAGUCAACUGG | 495 |
| 2481 | Sense | | |
| RTN4- | Anti- | UAUCACUAAAUAAGUCAACUGG | 496 |
| 2481 | Sense | | |
| RTN4- | Anti- | UAUCACUAAAUAAGUCAACUGG | 497 |
| 2481 | Sense | | |
| RTN4- | Anti- | UAUCACUAAAUAAGUCAACUGG | 498 |
| 2481 | Sense | | |
| RTN4- | Anti- | UAUCACUAAAUAAGUCAACUGG | 499 |
| 2481 | Sense | | |
| RTN4- | Anti- | UAUCACUAAAUAAGUCAACUGG | 500 |
| 2481 | Sense | | |
| RTN4- | Anti- | UAUCACUAAAUAAGUCAACUGG | 501 |
| 2481 | Sense | | |
| RTN4- | Anti- | UAUCACUAAAUAAGUCAACUGG | 502 |
| 2481 | Sense | | |
| RTN4- | Anti- | UAUCACUAAAUAAGUCAACUGG | 503 |
| 2481 | Sense | | |
| RTN4- | Sense | [ademAs-C16][mG][mU][mU][mG][mA][mC][fU][fU] | 504 |
| 2481 | | [fA][fU][mU][mU][mA][mG][mU][mG][mAs][mUs][mA] | |
| RTN4- | Sense | [ademAs-C16][mC][fU][fU][fA][fU][mU][mU][mA] | 505 |
| 2481 | | [mG][mU][mG][mAs][mUs][mA] | |
| RTN4- | Sense | [ademAs-C16][mG][mU][mU][mG][mA][mC][fU] | 506 |
| 2481 | | [fU][fA][fU][mU][mU][mA][mG][mUs][mGs][mA] | |
| RTN4- | Sense | [ademAs-C16][mC][fU][fU][fA][fU][mU][mU] | 507 |
| 2481 | | [mA][mG][mUs][mGs][mA] | |
| RTN4- | Sense | [ademAs-C18][mG][mU][mU][mG][mA][mC][fU][fU] | 508 |
| 2481 | | [fA][fU][mU][mU][mA][mG][mUs][mGs][mA] | |
| RTN4- | Sense | [ademAs-C22][mG][mU][mU][mG][mA][mC][fU] | 509 |
| 2481 | | [fU][fA][fU][mU][mU][mA][mG][mUs][mGs][mA] | |
| RTN4- | Sense | [ademAs-C16][mC][fU][fU][fA][fU][mU][mU][mA] | 510 |
| 2481 | | [mG][mU][mG][mAs][mUs][mA] | |
| RTN4- | Sense | [+G][mCs][mUs][ademAs-C16][mCs][mGs][mGs][+C][mC] | 511 |
| 2481 | | [mC][mA][mG][mU][mU][mG][mA][mC][fU][fU][fA][fU] | |
| | | [mU][mU][mA][mG][mUs][mGs][mA] | |
| RTN4- | Sense | [+G][mCs][mUs][mAs][mCs][mGs][mGs][+C][mC][mC][mA] | 512 |
| 2481 | | [mG][mU][mU][mG][mA][mC][fU][fU][fA][fU][mU][mU] | |
| | | [mA][mG][mUs][mGs][mA] | |
| RTN4- | Sense | [ademAs-C16][+G][mU][mU][mG][mA][mC][fU] | 513 |
| 2481 | | [fU][fA][fU][mU][mU][mA][+G][+T][mG][mAs][mUs][mA] | |
| RTN4- | Sense | [ademAs-C16][+C][fU][fU][fA][fU][mU][mU] | 514 |
| 2481 | | [mA][+G][+T][mG][mAs][mUs][mA] | |
| RTN4- | Anti- | [MePhosphonate-4O- | 515 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 516 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 517 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 518 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 519 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 520 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 521 |
| 2481 | Sense | mUs][fAs][fUs][fC][fA][mC][fU][mA][mA][fA][mU][mA] | |
| | | [mA][fGs][mUs][mCs][mAs][mAs][mCs][mUs][mGs][mG] | |
| RTN4- | Anti- | [MePhosphonate-4O- | 522 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 523 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 524 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| RTN4- | Anti- | [MePhosphonate-4O- | 525 |
| 2481 | Sense | mUs][fAs][fUs][fC][fA][mC][fU][mA][mA][fA][mU][mA] | |
| | | [mA][fG][mU][mC][mA][mA][mC][mUs][mGs][mG] | |
| Stem | N/A | GCAGCCGAAAGGCUGC | 526 |
| loop | | | |
| TUBB3- | Sense | UGAGAAUUGUGACUGAGCAGCCGAAAGGCUGC | 527 |
| 445 | | | |
| TUBB3- | Sense | AGAAUUGUGACUGAGCAGCCGAAAGGCUGC | 528 |
| 445 | | | |
| TUBB3- | Sense | AAUUGUGACUGAGCAGCCGAAAGGCUGC | 529 |
| 445 | | | |
| TUBB3- | Sense | [mUs][mG][mA][fG][fA][fA][fU][mU][mG][mU][mG][mA] | 530 |
| 445 | | [mC][mU][mG][mA][mG][mC][mA][mG][mC][mC][mG] | |
| | | [ademA-C16][mA][mA][mG][mG][mC][mU][mG][mC] | |
| TUBB3- | Sense | [+As][fG][fA][fA][fU][mU][mG][mU][mG][mA][mC][mU] | 531 |
| 445 | | [mG][mA][mG][mC][mA][mG][mC][mC][mG][ademA- | |
| | | C16][mA][mA][mG][mG][mC][mU][mG][mC] | |
| TUBB3- | Sense | [+As][fA][fU][mU][mG][mU][mG][mA][mC][mU][mG][mA] | 532 |
| 445 | | [mG][mC][mA][mG][mC][mC][mG][ademA- | |
| | | C16][mA][mA][mG][mG][mC][mU][mG][mC] | |
| TUBB3- | Anti- | [MePhosphonate-4O- | 533 |
| 445 | sense | mUs][fCs][fAs][fG][fU][mC][fA][mC][mA][fA][mU][mU] | |
| | | [mCs][fUs][mC][mA][mC][mA][mC][mUs][mGs][mG] | |
| TUBB3- | Anti- | [MePhosphonate-4O- | 534 |
| 445 | sense | mUs][fCs][fAs][fG][fU][mC][fA][mC][mA][fA][mU][mU] | |
| | | [mC][fU][mC][mAs][mCs][mAs][mCs][mUs][mGs][mG] | |
| TUBB3- | Anti- | [MePhosphonate-4O- | 535 |
| 445 | sense | mUs][fCs][fAs][fG][fU][mC][fA][mC][mA][fA][mU][mU] | |
| | | [mCs][fUs][mCs][mAs][mCs][mAs][mCs][mUs][mGs][mG] | |
| TUBB3- | Anti- | [MePhosphonate-4O- | 536 |
| 445 | sense | mUs][fCs][fAs][fG][fU][mC][fA][mC][mA][fA][mU][mUs] | |
| | | [mCs][fUs][mCs][mAs][mCs][mAs][mCs][mUs][mGs][mG] | |
| TUBB3- | Sense | AGAAUUGUGACUGA | 537 |
| 455 | | | |
| TUBB3- | Sense | AGAAUUGUGACTGA | 538 |
| 455 | | | |
| TUBB3- | Sense | [ademAs-C16][+G][mU][mG][mU][mG][mA][fG][fA] | 539 |
| 455 | | [[A][fU][mU][mG][mU][mG][mA][mC][mUs][mGs][mA] | |
| TUBB3- | Sense | [ademAs-C16][+G][mU][mG][mU][mG][mA][fG][fA] | 540 |
| 455 | | [fA][fU][mU][mG][mU][+G][mA][mC][mUs][mGs][mA] | |
| TUBB3- | Sense | [ademAs-C16][+G][mU][mG][mU][mG][mA][fG][fA] | 541 |
| 455 | | [fA][fU][mU][mG][mU][+G][+A][mC][mUs][mGs][mA] | |
| TUBB3- | Sense | [ademAs-C16][fG][fA][fA][fU][mU][mG][mU][mG] | 542 |
| 455 | | [mA][mC][mUs][mGs][mA] | |
| TUBB3- | Sense | [ademAs-C16][+G][fA][fA][fU][mU][mG][mU][mG] | 543 |
| 455 | | [mA][mC][mUs][mGs][mA] | |
| TUBB3- | Sense | [ademAs-C16][+G][fA][fA][fU][mU][mG][mU][+G] | 544 |
| 455 | | [mA][mC][mUs][mGs][mA] | |
| TUBB3- | Sense | [ademAs-C16][+G][fA][fA][fU][mU][mG][mU][+G] | 545 |
| 455 | | [+A][mC][mUs][mGs][mA] | |
| TUBB3- | Sense | [ademAs-C16][+G][A][fA][fU][mU][mG][mU][+G] | 546 |
| 455 | | [+A][mC][+Ts][+Gs][mA] | |
| TUBB3- | Sense | [mAs][mG][mU][mG][mU][mG][mA][fG][fA][fA][fU][mU] | 547 |
| 455 | | [mG][mU][mG][mA][mC][mUs][mGs][mA] | |
| TUBB3- | Sense | [ademAs-C16][+G][mU][mG][mU][mG][mA][fG][fA] | 548 |
| 455 | | [fA][fU][mU][mG][mU][+G][+A][mC][+Ts][mGs][mA] | |
| TUBB3- | Sense | UGAGAAUUGUGACUGA | 549 |
| 455 | | | |
| TUBB3- | Sense | UGAGAAUUGUGACTGA | 550 |
| 455 | | | |
| TUBB3- | Sense | [ademUs-C16][mG][mA][fG][fA][fA][fU][mU][mG] | 551 |
| 455 | | [mU][mG][mA][mC][mUs][mGs][mA] | |
| TUBB3- | Sense | [ademUs-C16][+G][mA][fG][fA][fA][fU][mU][mG] | 552 |
| 455 | | [mU][mG][mA][mC][mUs][mGs][mA] | |
| TUBB3- | Sense | [ademUs-C16][+G][mA][fG][fA][fA][fU][mU][mG] | 553 |
| 455 | | [mU][+G][mA][mC][mUs][mGs][mA] | |
| TUBB3- | Sense | [ademUs-C16][+G][mA][fG][fA][fA][fU][mU][mG] | 554 |
| 455 | | [mU][+G][+A][mC][mUs][mGs][mA] | |
| TUBB3- | Sense | [ademUs-C16][+G][mA][fG][fA][fA][fU][mU][mG] | 555 |
| 455 | | [mU][+G][+A][mC][+Ts][mGs][mA] | |
| TUBB3- | Sense | [ademUs-C16][+G][mA][fG][fA][fA][fU][mU] | 556 |
| 455 | | [mG][mU][+G][+A][mC][+Ts][+Gs][mA] | |
| TUBB3- | Sense | AAUUGUGACUGA | 557 |
| 455 | | | |
| TUBB3- | Sense | AAUUGUGACTGA | 558 |
| 455 | | | |
| TUBB3- | Sense | [ademAs-C16][mA][fU][mU][mG][mU][mG][mA] | 559 |
| 455 | | [mC][mUs][mGs][mA] | |
| TUBB3- | Sense | [ademAs-C16][+A][fU][mU][mG][mU][mG][mA] | 560 |
| 455 | | [mC][mUs][mGs][mA] | |
| TUBB3- | Sense | [ademAs-C16][+A][fU][mU][mG][mU][+G][mA] | 561 |
| 455 | | [mC][mUs][mGs][mA] | |
| TUBB3- | Sense | [ademAs-C16][+A][fU][mU][mG][mU][+G][+A] | 562 |
| 455 | | [mC][mUs][mGs][mA] | |
| TUBB3- | Sense | [ademAs-C16][+A][fU][mU][mG][mU][+G][+A] | 563 |
| 455 | | [mC][+Ts][mGs][mA] | |
| TUBB3- | Sense | [ademAs-C16][+A][fU][mU][mG][mU][+G][+A] | 564 |
| 455 | | [mC][+Ts][+Gs][mA] | |
| UGT8- | Sense | CAAUUAUGUUUGAAAGCCAA | 565 |
| 277 | | | |
| UGT8- | Sense | UGUUUGAAAGCCAA | 566 |
| 277 | | | |
| UGT8- | Sense | [ademCs-C16][mA][mA][mU][mU][mA][mU][fG] | 567 |
| 277 | | [fU][fU][fU][mG][mA][mA][mA][mG][mC][mCs][mAs][mA] | |
| UGT8- | Sense | [ademCs-C16][+A][mA][mU][mU][mA][mU][fG] | 568 |
| 277 | | [fU][fU][fU][mG][mA][mA][mA][mG][mC][mCs][mAs][mA] | |
| UGT8- | Sense | [ademCs-C16][+A][mA][mU][mU][mA][mU][fG] | 569 |
| 277 | | [fU][fU][fU][mG][mA][mA][+A][mG][mC][mCs][mAs][mA] | |
| UGT8- | Sense | [ademCs-C16][+A][mA][mU][mU][mA][mU][fG] | 570 |
| 277 | | [fU][fU][fU][mG][mA][mA][+A][+G][mC][mCs][mAs][mA] | |
| UGT8- | Sense | [ademUs-C16][fG][fU][fU][fU][mG][mA][mA] | 571 |
| 277 | | [mA][mG][mC][mCs][mAs][mA] | |
| UGT8- | Sense | [ademUs-C16][+G][fU][fU][fU][mG][mA][mA] | 572 |
| 277 | | [mA][mG][mC][mCs][mAs][mA] | |
| UGT8- | Sense | [ademUs-C16][+G][fU][fU][fU][mG][mA][mA] | 573 |
| 277 | | [+A][mG][mC][mCs][mAs][mA] | |
| UGT8- | Sense | [ademUs-C16][+G][fU][fU][fU][mG][mA][mA] | 574 |
| 277 | | [+A][+G][mC][mCs][mAs][mA] | |
| UGT8- | Sense | [ademUs-C16][+G][fU][fU][fU][mG][mA][mA] | 575 |
| 277 | | [+A][+G][mC][+Cs][+As][mA] | |
| UGT8- | Sense | [mCs][mA][mA][mU][mU][mA][mU][fG][fU][fU][fU][mG] | 576 |
| 277 | | [mA][mA][mA][mG][mC][mCs][mAs][mA] | |
| UGT8- | Anti- | UUGGCUUUCAAACAUAAUUGGG | 577 |
| 277 | sense | | |
| UGT8- | Anti- | [MePhosphonate-4O- | 578 |
| 277 | sense | mUs][fUs][fGs][fG][fC][mU][fU][mU][mC][fA][mA][mA] | |
| | | [mC][fA][mU][mA][mA][mU][mU][mGs][mGs][mG] | |
| UGT8- | Anti- | [MePhosphonate-4O- | 579 |
| 277 | sense | mUs][fUs][fGs][fG][fC][mU][fU][mU][mC][fA][mA][mA] | |
| | | [mCs][fAs][mU][mA][mA][mU][mU][mGs][mGs][mG] | |
| UGT8- | Sense | CAAUUAUGUUUGAAAGCCAAGCAGCCGAAAGGCUG | 580 |
| 277 | | C | |
| UGT8- | Sense | [mCs][mA][mA][mU][mU][mA][mU][fG][fU][fU][fU][mG] | 581 |
| 277 | | [mA][mA][mA][mG][mC][mC][mA][mA][mG][mC][mA][mG] | |
| | | [mC][mC][mG][ademA-C16][mA][mA][mG][mG][mC] | |
| | | [mU][mG][mC] | |
| GFAP- | Sense | AGGAUCUACUCAACGUUAAAGCAGCCGAAAGGCUG | 582 |
| 1147 | | C | |
| GFAP- | Sense | AGGAUCUACUCAACGUUAAA | 583 |
| 1147 | | | |
| GFAP- | Sense | UACUCAACGUUAAA | 584 |
| 1147 | | | |
| GFAP- | Sense | [mAs][mG][mG][mA][mU][mC][mU][fA][fC][fU][fC][mA] | 585 |
| 1147 | | [mA][mC][mG][mU][mU][mA][mA][mA][mG][mC][mA][mG] | |
| | | [mC][mC][mG][ademA-C16][mA][mA][mG] | |
| | | [mG][mC][mU][mG][mC] | |
| GFAP- | Sense | [ademAs-C16][mG][mG][mA][mU][mC][mU][fA] | 586 |
| 1147 | | [fC][fU][fC][mA][mA][mC][mG][mU][mU][mAs][mAs][mA] | |
| GFAP- | Sense | [ademAs-C16][+G][mG][mA][mU][mC][mU][A][fC] | 587 |
| 1147 | | [fU][fC][mA][mA][mC][mG][mU][mU][mAs][mAs][mA] | |
| GFAP- | Sense | [ademAs-C16][+G][mG][mA][mU][mC][mU][fA][fC] | 588 |
| 1147 | | [fU][fC][mA][mA][mC][+G][mU][mU][mAs][mAs][mA] | |
| GFAP- | Sense | [ademAs-C16][+G][mG][mA][mU][mC][mU][fA][fC] | 589 |
| 1147 | | [fU][fC][mA][mA][mC][+G][+]][mU][mAs][mAs][mA] | |
| GFAP- | Sense | [ademUs-C16][fA][fC][fU][fC][mA][mA][mC][mG] | 590 |
| 1147 | | [mU][mU][mAs][mAs][mA] | |
| GFAP- | Sense | [ademUs-C16][+A][fC][fU][fC][mA][mA][mC][mG] | 591 |
| 1147 | | [mU][mU][mAs][mAs][mA] | |
| GFAP- | Sense | [ademUs-C16][+A][fC][fU][fC][mA][mA][mC][+G] | 592 |
| 1147 | | [mU][mU][mAs][mAs][mA] | |
| GFAP- | Sense | [ademUs-C16][+A][fC][fU][fC][mA][mA][mC][+G] | 593 |
| 1147 | | [+T][mU][mAs][mAs][mA] | |
| GFAP- | Sense | [ademUs-C16][+A][fC][fU][fC][mA][mA][mC][+G] | 594 |
| 1147 | | [+T][mU][+As][+As][mA] | |
| GFAP- | Sense | [mAs][mG][mG][mA][mU][mC][mU][fA][fC][fU][fC][mA] | 595 |
| 1147 | | [mA][mC][mG][mU][mU][mAs][mAs][mA] | |
| GFAP- | Anti- | UUUAACGUUGAGUAGAUCCUGG | 596 |
| 1147 | sense | | |
| GFAP- | Anti- | [MePhosphonate-4O- | 597 |
| 1147 | sense | mUs][fUs][fUs][fA][fA][mC][fG][mU][mU][fG][mA][mG] | |
| | | [mU][fA][mG][mA][mU][mC][mC][mUs][mGs][mG] | |
| GFAP- | Anti- | [MePhosphonate-4O- | 598 |
| 1147 | sense | mUs][fUs][fUs][fA][fA][mC][fG][mU][mU][fG][mA][mG] | |
| | | [mUs][fAs][mG][mA][mU][mC][mC][mUs][mGs][mG] | |
| TUBB3- | Sense | [mAs][ademG- | 599 |
| 445 | | C16][mU][mG][mU][mG][mA][fG][fA][fA][fU][mU][mG] | |
| | | [mU][mG][mA][mC][mUs][mGs][mA] | |
| TUBB3- | Sense | [mAs][mG][ademU- | 600 |
| 445 | | C16][mG][mU][mG][mA][fG][fA][fA][fU][mU][mG][mU] | |
| | | [mG][mA][mC][mUs][mGs][mA] | |
| TUBB3- | Sense | [mAs][mG][mU][ademG- | 601 |
| 445 | | C16][mU][mG][mA][fG][fA][fA][fU][mU][mG][mU][mG] | |
| | | [mA][mC][mUs][mGs][mA] | |
| TUBB3- | Sense | [mAs][mG][mU][mG][ademU- | 602 |
| 445 | | C16][mG][mA][fG][fA][fA][fU][mU][mG][mU][mG][mA] | |
| | | [mC][mUs][mGs][mA] | |
| TUBB3- | Sense | [mAs][mG][mU][mG][mU][ademG- | 603 |
| 445 | | C16][mA][fG][fA][fA][fU][mU][mG][mU][mG][mA][mC] | |
| | | [mUs][mGs][mA] | |
| TUBB3- | Sense | [mAs][mG][mU][mG][mU][mG][ademA- | 604 |
| 445 | | C16][fG][fA][fA][fU][mU][mG][mU][mG][mA][mC][mUs] | |
| | | [mGs][mA] | |
| TUBB3- | Sense | [mAs][mG][mU][mG][mU][mG][mA][ademG- | 605 |
| 445 | | C16][fA][fA][fU][mU][mG][mU][mG][mA][mC][mUs][mGs] | |
| | | [mA] | |
| TUBB3- | Sense | [mAs][mG][mU][mG][mU][mG][mA][mG][ademA-C16] | 606 |
| 445 | | [fA][fU][mU][mG][mU][mG][mA][mC][mUs][mGs][mA] | |
| TUBB3- | Sense | [mUs][ademG-C16][mA][fG][fA][fA][fU][mU][mG] | 607 |
| 445 | | [mU][mG][mA][mC][mUs][mGs][mA] | |
| TUBB3- | Sense | [mUs][mG][ademA-C16][fG][fA][fA][fU][mU][mG][mU] | 608 |
| 445 | | [mG][mA][mC][mUs][mGs][mA] | |
| TUBB3- | Sense | [mUs][mG][mA][ademG-C16][fA][fA][fU][mU][mG][mU] | 609 |
| 445 | | [mG][mA][mC][mUs][mGs][mA] | |
| TUBB3- | Sense | [mUs][mG][mA][fG][ademA-C16][fA][fU][mU][mG][mU] | 610 |
| 445 | | [mG][mA][mC][mUs][mGs][mA] | |
| TUBB3- | Sense | [mAs][ademG-C16][fA][fA][fU][mU][mG][mU] | 611 |
| 445 | | [mG][mA][mC][mUs][mGs][mA] | |
| TUBB3- | Sense | [mAs][mG][ademA-C16][fA][fU][mU][mG][mU][mG] | 612 |
| 445 | | [mA][mC][mUs][mGs][mA] | |
| TUBB3- | Sense | [ademAs-C16][fA][fU][mU][mG][mU][mG] | 613 |
| 445 | | [mA][mC][mUs][mGs][mA] | |
| GFAP- | Sense | AGGAUCUACUCAACGTUAAA | 614 |
| 1147 | | | |
| GFAP- | Sense | UCUACUCAACGTUAAA | 615 |
| 1147 | | | |
| GFAP- | Sense | ATCUACUCAACGTUAA | 616 |
| 1147 | | | |
| GFAP- | Sense | GAUCUACUCAACGTUA | 617 |
| 1147 | | | |
| GFAP- | Sense | GGAUCUACUCAACGTU | 618 |
| 1147 | | | |
| GFAP- | Sense | AGGAUCUACUCAACGT | 619 |
| 1147 | | | |
| GFAP- | Sense | UACUCAACGTUAAA | 620 |
| 1147 | | | |
| GFAP- | Sense | CTACUCAACGTUAA | 621 |
| 1147 | | | |
| GFAP- | Sense | UCUACUCAACGTUA | 622 |
| 1147 | | | |
| GFAP- | Sense | ATCUACUCAACGTU | 623 |
| 1147 | | | |
| GFAP- | Sense | GAUCUACUCAACGT | 624 |
| 1147 | | | |
| GFAP- | Sense | CTCAACGTUAAA | 625 |
| 1147 | | | |
| GFAP- | Sense | ACUCAACGTUAA | 626 |
| 1147 | | | |
| GFAP- | Sense | UACUCAACGTUA | 627 |
| 1147 | | | |
| GFAP- | Sense | CTACUCAACGTU | 628 |
| 1147 | | | |
| GFAP- | Sense | UCUACUCAACGT | 629 |
| 1147 | | | |
| TUBB3- | Sense | GTGAGAAUUGUGACUG | 630 |
| 0445 | | | |
| TUBB3- | Sense | UGUGAGAAUUGUGACU | 631 |
| 0445 | | | |
| TUBB3- | Sense | GTGUGAGAAUUGUGAC | 632 |
| 0445 | | | |
| TUBB3- | Sense | GAGAAUUGUGACUG | 633 |
| 0445 | | | |
| TUBB3- | Sense | UGAGAAUUGUGACU | 634 |
| 0445 | | | |
| TUBB3- | Sense | GTGAGAAUUGUGAC | 635 |
| 0445 | | | |
| TUBB3- | Sense | UGUGAGAAUUGUGA | 636 |
| 0445 | | | |
| TUBB3- | Sense | GAAUUGUGACUG | 637 |
| 0445 | | | |
| TUBB3- | Sense | AGAAUUGUGACU | 638 |
| 0445 | | | |
| TUBB3- | Sense | GAGAAUUGUGAC | 639 |
| 0445 | | | |
| TUBB3- | Sense | UGAGAAUUGUGA | 640 |
| 0445 | | | |
| GFAP- | Sense | UCUACUCAACGUUAAA | 641 |
| 1147 | | | |
| GFAP- | Sense | UCUACUCAACGUUA | 642 |
| 1147 | | | |
| GFAP- | Sense | UACUCAACGUUA | 643 |
| 1147 | | | |
| TUBB3- | Sense | AGUGUGAGAAUUGTGACUGA | 644 |
| 0445 | | | |
| TUBB3- | Sense | UTGUGACUGA | 665 |
| 0445 | | | |
| TUBB3- | Sense | ATUGUGACUG | 666 |
| 0445 | | | |
| TUBB3- | Sense | AAUUGUGACU | 667 |
| 0445 | | | |
| TUBB3- | Sense | GAAUUGUGAC | 668 |
| 0445 | | | |
| TUBB3- | Sense | AGAAUUGUGA | 669 |
| 0445 | | | |
| TUBB3- | Sense | GAGAAUUGTG | 670 |
| 0445 | | | |
| TUBB3- | Sense | UGAGAAUUGT | 671 |
| 0445 | | | |
| GFAP- | Sense | [ademUs-C16][+C][mU][fA][fC][fU][fC][mA] | 672 |
| 1147 | | [mA][mC][+G][+T][mU][mAs][mAs][mA] | |
| GFAP- | Sense | [ademAs-C16][+T][mC][mU][fA][fC][fU][fC] | 673 |
| 1147 | | [mA][mA][mC][+G][+T][mUs][mAs][mA] | |
| GFAP- | Sense | [ademGs-C16][+A][mU][mC][mU][fA][fC][fU] | 674 |
| 1147 | | [fC][mA][mA][mC][+G][+Ts][mUs][mA] | |
| GFAP- | Sense | [ademGs-C16][+G][mA][mU][mC][mU][fA][fC] | 675 |
| 1147 | | [fU][fC][mA][mA][mC][+Gs][+Ts][mU] | |
| GFAP- | Sense | [ademAs-C16][+G][mG][mA][mU][mC][mU][fA] | 676 |
| 1147 | | [fC][fU][fC][mA][mA][mCs][+Gs][+T] | |
| GFAP- | Sense | [ademCs-C16][+T][fA][fC][fU][fC][mA][mA][mC] | 677 |
| 1147 | | [+G][+T][mUs][mAs][mA] | |
| GFAP- | Sense | [ademUs-C16][+C][mU][fA][fC][fU][fC][mA] | 678 |
| 1147 | | [mA][mC][+G][+Ts][mUs][mA] | |
| GFAP- | Sense | [ademAs-C16][+]][mC][mU][fA][fC][fU][fC][mA] | 679 |
| 1147 | | [mA][mC][+Gs][+Ts][mU] | |
| GFAP- | Sense | [ademGs-C16][+A][mU][mC][mU][fA][fC][fU][fC] | 680 |
| 1147 | | [mA][mA][mCs][+Gs][+T] | |
| GFAP- | Sense | [ademCs-C16][+T][fC][mA][mA][mC][+G][+T][mU] | 681 |
| 1147 | | [mAs][mAs][mA] | |
| GFAP- | Sense | [ademAs-C16][+C][fU][fC][mA][mA][mC][+G][+T] | 682 |
| 1147 | | [mUs][mAs][mA] | |
| GFAP- | Sense | [ademUs-C16][+A][fC][fU][fC][mA][mA][mC] | 683 |
| 1147 | | [+G][+Ts][mUs][mA] | |
| GFAP- | Sense | [ademCs-C16][+T][fA][fC][fU][fC][mA][mA][mC] | 684 |
| 1147 | | [+Gs][+Ts][mU] | |
| GFAP- | Sense | [ademUs-C16][+C][mU][fA][fC][fU][fC][mA][mA] | 685 |
| 1147 | | [mCs][+Gs][+T] | |
| TUBB3- | Sense | [ademGs-C16][+T][mG][mA][fG][fA][fA][fU][mU][mG] | 686 |
| 0445 | | [mU][+G][+A][mCs][mUs][mG] | |
| TUBB3- | Sense | [ademUs-C16][+G][mU][mG][mA][fG][fA][fA][fU][mU] | 687 |
| 0445 | | [mG][mU][+G][+As][mCs][mU] | |
| TUBB3- | Sense | [ademGs-C16][+T][mG][mU][mG][mA][fG][fA][fA][fU] | 688 |
| 0445 | | [mU][mG][mU][+Gs][+As][mC] | |
| TUBB3- | Sense | [ademGs-C16][+A][fG][fA][fA][fU][mU][mG][mU] | 689 |
| 0445 | | [+G][+A][mCs][mUs][mG] | |
| TUBB3- | Sense | [ademUs-C16][+G][mA][fG][fA][fA][fU][mU][mG] | 690 |
| 0445 | | [mU][+G][+As][mCs][mU] | |
| TUBB3- | Sense | [ademGs-C16][+T][mG][mA][fG][fA][fA][fU][mU] | 691 |
| 0445 | | [mG][mU][+Gs][+As][mC] | |
| TUBB3- | Sense | [ademUs-C16][+G][mU][mG][mA][fG][fA][fA] | 692 |
| 0445 | | [fU][mU][mG][mUs][+Gs][+A] | |
| TUBB3- | Sense | [ademGs-C16][+A][fA][fU][mU][mG][mU][+G][+A] | 693 |
| 0445 | | [mCs][mUs][mG] | |
| TUBB3- | Sense | [ademAs-C16][+G][fA][fA][fU][mU][mG][mU][+G] | 694 |
| 0445 | | [+As][mCs][mU] | |
| TUBB3- | Sense | [ademGs-C16][+A][fG][fA][fA][fU][mU][mG][mU] | 695 |
| 0445 | | [+Gs][+As][mC] | |
| TUBB3- | Sense | [ademUs-C16][+G][mA][fG][fA][fA][fU][mU] | 696 |
| 0445 | | [mG][mUs][+Gs][+A] | |
| GFAP- | Sense | [ademAs-C16][mG][mG][mA][mU][+C][mU][fA] | 697 |
| 1147 | | [fC][fU][fC][mA][mA][mC][+G][+T][mU][mAs][mAs][mA] | |
| GFAP- | Sense | [ademUs-C16][mC][mU][fA][fC][fU][fC][mA] | 698 |
| 1147 | | [mA][mC][mG][mU][mU][mAs][mAs][mA] | |
| GFAP- | Sense | [mUs][mC][ademU-C16][fA][fC][fU][fC][mA][mA][mC] | 699 |
| 1147 | | [mG][mU][mU][mAs][mAs][mA] | |
| GFAP- | Sense | [mUs][mC][mU][fA][ademC-C16][fU][fC][mA][mA][mC] | 700 |
| 1147 | | [mG][mU][mU][mAs][mAs][mA] | |
| GFAP- | Sense | [ademUs-C16][mC][mU][fA][fC][fU][fC] | 701 |
| 1147 | | [mA][mA][mC][mG][mUs][mUs][mA] | |
| GFAP- | Sense | [mUs][mC][ademU-C16][fA][fC][fU][fC][mA][mA] | 702 |
| 1147 | | [mC][mG][mUs][mUs][mA] | |
| GFAP- | Sense | [mUs][mC][mU][fA][ademC-C16][fU][fC][mA] | 703 |
| 1147 | | [mA][mC][mG][mUs][mUs][mA] | |
| GFAP- | Sense | [mUs][+C][ademU-C16][fA][fC][fU][fC][mA][mA] | 704 |
| 1147 | | [mC][+G][+T][mU][mAs][mAs][mA] | |
| GFAP- | Sense | [mUs][+C][mU][fA][ademC-C16][fU][fC][mA][mA][mC] | 705 |
| 1147 | | [+G][+T][mU][mAs][mAs][mA] | |
| GFAP- | Sense | [mUs][+C][ademU-C16][fA][fC][fU][fC] | 706 |
| 1147 | | [mA][mA][mC][+G][+Ts][mUs][mA] | |
| GFAP- | Sense | [mUs][+C][mU][fA][ademC-C16][fU][fC][mA][mA] | 707 |
| 1147 | | [mC][+G][+Ts][mUs][mA] | |
| GFAP- | Sense | [ademAs-C16][mG][mG][mA][mU][mC][mU][+A] | 708 |
| 1147 | | [fC][fU][fC][mA][mA][mC][+G][+T][mU][mAs][mAs][mA] | |
| GFAP- | Sense | [ademUs-C16][fA][fC][fU][fC][mA][mA][mC][mG] | 709 |
| 1147 | | [mU][mU][mAs][mAs][mA] | |
| GFAP- | Sense | [mU][fA][ademC-C16][fU][fC][mA][mA][mC][mG][mU] | 710 |
| 1147 | | [mU][mAs][mAs][mA] | |
| GFAP- | Sense | [mUs][+A][ademC-C16][fU][fC][mA][mA][mC][+G][+T] | 711 |
| 1147 | | [mU][mAs][mAs][mA] | |
| GFAP- | Sense | [ademUs-C16][fA][fC][fU][fC][mA][mA][mC] | 712 |
| 1147 | | [mG][mUs][mUs][mA] | |
| GFAP- | Sense | [mU][fA][ademC-C16][fU][fC][mA][mA][mC][mG][mUs] | 713 |
| 1147 | | [mUs][mA] | |
| GFAP- | Sense | [mUs][+A][ademC-C16][fU][fC][mA][mA][mC][+G][+Ts] | 714 |
| 1147 | | [mUs][mA] | |
| TUBB3- | Sense | [ademAs-C16][mG][mU][mG][mU][+G][mA][fG] | 715 |
| 0445 | | [fA][fA][fU][mU][mG][mU][+G][+A][mC][mUs][mGs][mA] | |
| TUBB3- | Sense | [ademUs-C16][mG][mA][fG][fA][fA][fU][mU][mG] | 716 |
| 0445 | | [mU][mG][mAs][mCs][mU] | |
| TUBB3- | Sense | [mUs][mG][ademA-C16][fG][fA][fA][fU][mU][mG] | 717 |
| 0445 | | [mU][mG][mAs][mCs][mU] | |
| TUBB3- | Sense | [mUs][mG][mA][fG][ademA- | 718 |
| 0445 | | C16][fA][fU][mU][mG][mU][mG][mAs][mCs][mU] | |
| TUBB3- | Sense | [mUs][+G][ademA-C16][fG][fA][fA][fU][mU][mG] | 719 |
| 0445 | | [mU][+G][+A][mC][mUs][mGs][mA] | |
| TUBB3- | Sense | [mUs][+G][mA][fG][ademA-C16][fA][fU][mU][mG][mU] | 720 |
| 0445 | | [+G][+A][mC][mUs][mGs][mA] | |
| TUBB3- | Sense | [mUs][+G][ademA-C16][fG][fA][fA][fU][mU] | 721 |
| 0445 | | [mG][mU][+G][+As][mCs][mU] | |
| TUBB3- | Sense | [mUs][+G][mA][fG][ademA-C16][fA][fU][mU][mG] | 722 |
| 0445 | | [mU][+G][+As][mCs][mU] | |
| TUBB3- | Sense | [ademAs-C16][mG][mU][mG][mU][mG][mA][+G] | 723 |
| 0445 | | [fA][fA][fU][mU][mG][mU][+G][+A][mC][mUs][mGs][mA] | |
| TUBB3- | Sense | [mAs][+G][ademA-C16][fA][fU][mU][mG][mU][+G] | 724 |
| 0445 | | [+A][mC][mUs][mGs][mA] | |
| TUBB3- | Sense | [ademAs-C16][fG][fA][fA][fU][mU][mG][mU] | 725 |
| 0445 | | [mG][mAs][mCs][mU] | |
| TUBB3- | Sense | [mA][fG][ademA-C16][fA][fU][mU][mG][mU][mG] | 726 |
| 0445 | | [mAs][mCs][mU] | |
| TUBB3- | Sense | [ademAs-C16][+G][fA][fA][fU][mU][mG][mU] | 727 |
| 0445 | | [+G][+As][mCs][mU] | |
| TUBB3- | Sense | [mAs][+G][ademA-C16][fA][fU][mU][mG][mU] | 728 |
| 0445 | | [+G][+As][mCs][mU] | |
| TUBB3- | Sense | [ademAs-C16][+G][mU][mG][mU][mG][mA][fG] | 729 |
| 0445 | | [fA][fA][fU][mU][mG][+T][+G][mA][mC][mUs][mGs][mA] | |
| TUBB3- | Sense | [ademAs-C16][+G][mU][mG][mU][mG][mA][fG] | 730 |
| 0445 | | [fA][fA][fU][mU][+G][+T][mG][mA][mC][mUs][mGs][mA] | |
| TUBB3- | Sense | [ademUs-C16][+T][mG][mU][+G][+A][mC] | 731 |
| 0445 | | [mUs][mGs][mA] | |
| TUBB3- | Sense | [ademAs-C16][+T][mU][mG][mU][+G][+A] | 732 |
| 0445 | | [mCs][mUs][mG] | |
| TUBB3- | Sense | [ademAs-C16][+A][fU][mU][mG][mU][+G][+As][mCs][mU] | 733 |
| 0445 | | | |
| TUBB3- | Sense | [ademGs-C16][+A][fA][fU][mU][mG][mU][+Gs][+As][mC] | 734 |
| 0445 | | | |
| TUBB3- | Sense | [ademAs-C16][+G][[A][fA][fU][mU][mG][mUs][+Gs][+A] | 735 |
| 0445 | | | |
| TUBB3- | Sense | [ademGs-C16][+A][fG][fA][fA][fU][mU][mGs][+Ts][+G] | 736 |
| 0445 | | | |
| TUBB3- | Sense | [ademUs-C16][+G][mA][fG][fA][fA][fU][mUs][+Gs][+T] | 737 |
| 0445 | | | |
| UGT8- | Sense | CAAUUAUGUUUGAAAGC | 738 |
| 277 | | | |
| UGT8- | Sense | UGUUUGAAAGC | 739 |
| 277 | | | |
| UGT8- | Sense | [ademCs-C16][mA][mA][mU][mU][mA][mU] | 740 |
| 277 | | [fG][fU][fU][fU][mG][mA][mA][mAs][mGs][mC] | |
| UGT8- | Sense | [ademUs-C16][+G][fU][fU][fU][mG][mA][mA] | 741 |
| 277 | | [mAs][mGs][mC] | |
| ALDH2 | Sense | GGUGGAUGAAACUCAGUUUA | 742 |
| ALDH2 | Sense | GGUGGAUGAAACUCAGUUU | 743 |
| ALDH2 | Sense | GGUGGAUGAAACUCAGUU | 744 |
| ALDH2 | Sense | GGUGGAUGAAACUCAGU | 745 |
| ALDH2 | Sense | GGUGGAUGAAACUCAG | 746 |
| ALDH2 | Sense | [ademGs- | 747 |
| | | C22][mG][mU][mG][mG][mA][mU][fG][fA][fA][fA][mC][mU] | |
| | | [mC][mA][mG][mU][mUs][mUs][mA] | |
| ALDH2 | Sense | [ademGs- | 748 |
| | | C22][mG][mU][mG][mG][mA][mU][fG][fA][fA][fA][mC][mU] | |
| | | [mC][mA][mG][mUs][mUs][mU] | |
| ALDH2 | Sense | [ademGs- | 749 |
| | | C22][mG][mU][mG][mG][mA][mU][fG][fA][fA][fA][mC][mU] | |
| | | [mC][mA][mGs][mUs][mU] | |
| ALDH2 | Sense | [ademGs- | 750 |
| | | C22][mG][mU][mG][mG][mA][mU][fG][fA][fA][fA][mC][mU] | |
| | | [mC][mAs][mGs][mU] | |
| ALDH2 | Sense | [ademGs- | 751 |
| | | C22][mG][mU][mG][mG][mA][mU][fG][fA][fA][fA][mC][mU] | |
| | | [mCs][mAs][mG] | |
| ALDH2 | Anti- | [MePhosphonate-4O- | 752 |
| | sense | mUs][fAs][fAs][fA][fC][mU][fG][mA][mG][fU][mU][mU] | |
| | | [mC][fA][mU][mC][mC][mA][mC][mCs][mGs][mG] | |
| ALDH2 | Anti- | [MePhosphonate-4O- | 753 |
| | sense | mUs][fAs][fAs][fAs][fC][mU][fG][mA][mG][fU][mU][mU] | |
| | | [mC][fA][mU][mC][mC][mA][mC][mCs][mGs][mG] | |

SEQUENCE LISTING-II

| | | | | SEQ |
|---|---|---|---|---|
| | | | | ID |
| Name | Description | Ligand | Sequence | NO |
| GFAP- | 19 mer sense | N/A | AGGAUCUACUCAACGUUAA | 754 |
| 1147 | strand | | | |
| GFAP- | 19 mer | N/A | UUAACGUUGAGUAGAUCCU | 755 |
| 1147 | antisense | | | |
| | strand | | | |
| GFAP- | 36 mer sense | N/A | AGGAUCUACUCAACGUUAAAGCAGCCGAA | 756 |
| 1147 | strand | | AGGCUGC | |
| GFAP- | 36 mer | N/A | UUUAACGUUGAGUAGAUCCUGG | 757 |
| 1147 | antisense | | | |
| | strand | | | |
| GFAP- | Modified | GalNAc | [mAs][mG][mG][mA][mU][mC][mU][fA][fC][fU] | 758 |
| 1147 | 36 mer sense | | [fC][mA][mA][mC][mG][mU][mU][mA][mA][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| GFAP- | Modified | Tetraloop | [ademAs-C16][mG][mG][mA][mU][mC][mU] | 759 |
| 1147 | 36 mer sense | P1 C16 | [fA][fC][fU][fC][mA][mA][mC][mG][mU][mU] | |
| | strand | | [mA][mA][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][ademA-C16][mU][mC][mU] | 760 |
| 1147 | 36 mer sense | P4 C16 | [fA][fC][fU][fC][mA][mA][mC][mG][mU][mU] | |
| | strand | | [mA][mA][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][mA][mU][mC][mU][ademA- | 761 |
| 1147 | 36 mer sense | P8 C16 | C16][fC][fU][fC][mA][mA][mC][mG][mU][mU] | |
| | strand | | [mA][mA][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][mA][mU][mC][mU][fA][fC][fU] | 762 |
| 1147 | 36 mer sense | P12 C16 | [fC][ademA-C16][mA][mC][mG][mU][mU] | |
| | strand | | [mA][mA][mA][mG][mC][mA][mG][mC][mC] | |
| | | | [mG][mA][mA][mA][mG][mG][mC][mU][mG][mC] | |
| GFAP- | Modified | Tetraloop | [mAs][mG][mG][mA][mU][mC][mU][fA][fC][fU] | 763 |
| 1147 | 36 mer sense | P13 C16 | [fC][mA][ademA-C16][mC][mG][mU][mU][mA] | |
| | strand | | [mA][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][mA][mU][mC][mU][fA][fC][fU] | 764 |
| 1147 | 36 mer sense | P18 C16 | [fC][mA][mA][mC][mG][mU][mU][ademA-C16] | |
| | strand | | [mA][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][mA][mU][mC][mU][fA][fC][fU] | 765 |
| 1147 | 36 mer sense | P19 C16 | [fC][mA][mA][mC][mG][mU][mU][mA][ademA- | |
| | strand | | C16][mA][mG][mC][mA][mG][mC][mC][mG] | |
| | | | [mA][mA][mA][mG][mG][mC][mU][mG][mC] | |
| GFAP- | Modified | Tetraloop | [mAs][mG][mG][mA][mU][mC][mU][fA][fC][fU] | 766 |
| 1147 | 36 mer sense | P20 C16 | [fC][mA][mA][mC][mG][mU][mU][mA][mA] | |
| | strand | | [ademA- | |
| | | | C16][mG][mC][mA][mG][mC][mC][mG][mA] | |
| | | | [mA][mA][mG][mG][mC][mU][mG][mC] | |
| GFAP- | Modified | Tetraloop | [mAs][mG][mG][mA][mU][mC][mU][A][fC][fU] | 767 |
| 1147 | 36 mer sense | P23 C16 | [fC][mA][mA][mC][mG][mU][mU][mA][mA][mA] | |
| | strand | | [mG][mC][ademA- | |
| | | | C16][mG][mC][mC][mG][mA] | |
| | | | [mA][mA][mG][mG][mC][mU][mG][mC] | |
| GFAP- | Modified | Tetraloop | [mAs][mG][mG][mA][mU][mC][mU][A][fC][fU] | 768 |
| 1147 | 36 mer sense | P28 C16 | [fC][mA][mA][mC][mG][mU][mU][mA][mA] | |
| | strand | | [mA][mG][mC][mA][mG][mC][mC][mG][ademA- | |
| | | | C16] | |
| | | | [mA][mA][mG][mG][mC][mU][mG][mC] | |
| GFAP- | Modified | Tetraloop | [mAs][mG][mG][mA][mU][mC][mU][fA][fC][fU] | 769 |
| 1147 | 36 mer sense | P29 C16 | [fC][mA][mA][mC][mG][mU][mU][mA][mA] | |
| | strand | | [mA][mG][mC][mA][mG][mC][mC][mG][mA][ademA- | |
| | | | C16][mA][mG][mG][mC][mU][mG][mC] | |
| GFAP- | Modified | Tetraloop | [mAs][mG][mG][mA][mU][mC][mU][fA][fC][fU] | 770 |
| 1147 | 36 mer sense | P30 C16 | [fC][mA][mA][mC][mG][mU][mU][mA][mA] | |
| | strand | | [mA][mG][mC][mA][mG][mC][mC][mG][mA][mA] | |
| | | | [ademA-C16][mG][mG][mC][mU][mG][mC] | |
| GFAP- | Modified | N/A | [MePhosphonate-4O-mUs][fUs][fUs][fA][fA][mC] | 771 |
| 1147 | 22 mer | | [fG][mU][mU][fG][mA][mG][mU][fA][mG][mA] | |
| | antisense | | [mU][mC][mC][mUs][mGs][mG] | |
| | strand | | | |
| GFAP- | 20 mer sense | N/A | AGGAUCUACUCAACGUUAAA | 772 |
| 1147 | strand | | | |
| GFAP- | Modified | Blunt- | [ademAs-C16][mG][mG][mA][mU][mC][mU][fA] | 773 |
| 1147 | 20 mer sense | End P1 | [fC][fU][fC][mA][mA][mC][mG][mU][mU][mAs] | |
| | strand | C16 | [mAs][mA] | |
| GFAP- | Modified | Blunt- | [mAs][mG][mG][ademA-C16][mU][mC][mU][fA] | 774 |
| 1147 | 20 mer sense | End | [fC][fU][fC][mA][mA][mC][mG][mU][mU][mAs] | |
| | strand | P4 C16 | [mAs][mA] | |
| GFAP- | Modified | Blunt- | [mAs][mG][mG][mA][mU][mC][mU][ademA- | 775 |
| | 20 mer sense | End | C16] | |
| 1147 | strand | P8 C16 | [fC][fU][fC][mA][mA][mC][mG][mU][mU][mAs] | |
| | | | [mAs][mA] | |
| GFAP- | Modified | Blunt- | [mAs][mG][mG][mA][mU][mC][mU][fA][fC][fU] | 776 |
| 1147 | 20 mer sense | End | [fC][ademA-C16][mA][mC][mG][mU][mU][mAs] | |
| | strand | P12 | [mAs][mA] | |
| | | C16 | | |
| GFAP- | Modified | Blunt- | [mAs][mG][mG][mA][mU][mC][mU][fA][fC][fU] | 777 |
| 1147 | 20 mer sense | End | [fC][mA][ademA-C16][mC][mG][mU][mU][mAs] | |
| | strand | P13 | [mAs][mA] | |
| | | C16 | | |
| GFAP- | Modified | Blunt- | [mAs][mG][mG][mA][mU][mC][mU][fA][fC][fU] | 778 |
| 1147 | 20 mer sense | End | [fC][mA][mA][mC][mG][mU][mU][ademAs-C16] | |
| | strand | P18 | [mAs][mA] | |
| | | C16 | | |
| GFAP- | Modified | Blunt- | [mAs][mG][mG][mA][mU][mC][mU][fA][fC][fU] | 779 |
| 1147 | 20 mer sense | End | [fC][mA][mA][mC][mG][mU][mU][mAs][ademAs- | |
| | strand | P19 | C16][mA] | |
| | | C16 | | |
| GFAP- | Modified | Blunt- | [mAs][mG][mG][mA][mU][mC][mU][fA][fC][fU] | 780 |
| 1147 | 20 mer sense | End | [fC][mA][mA][mC][mG][mU][mU][mAs][mAs] | |
| | strand | P20 | [ademA-C16] | |
| | | C16 | | |
| Trans- | GFAP | N/A | AGAGCCAGAGCAGGATGGAGAGGAGACGC | 781 |
| cript | Transcript | | ATCACCTCCGCTGCTCGCCGCTCCTACGTCT | |
| Variant | variant 1 | | CCTCAGGGGAGATGATGGTGGGGGGCCTGG | |
| 1 | | | CTCCTGGCCGCCGTCTGGGTCCTGGCACCC | |
| | | | GCCTCTCCCTGGCTCGAATGCCCCCTCCACT | |
| | | | CCCGACCCGGGTGGATTTCTCCCTGGCTGG | |
| | | | GGCACTCAATGCTGGCTTCAAGGAGACCCG | |
| | | | GGCCAGTGAGCGGGCAGAGATGATGGAGC | |
| | | | TCAATGACCGCTTTGCCAGCTACATCGAGA | |
| | | | AGGTTCGCTTCCTGGAACAGCAAAACAAGG | |
| | | | CGCTGGCTGCTGAGCTGAACCAGCTGCGGG | |
| | | | CCAAGGAGCCCACCAAGCTGGCAGACGTCT | |
| | | | ACCAGGCTGAGCTGCGAGAGCTGCGGCTGC | |
| | | | GGCTCGATCAACTCACCGCCAACAGCGCCC | |
| | | | GGCTGGAGGTTGAGAGGGACAATCTGGCAC | |
| | | | AGGACCTGGCCACTGTGAGGCAGAAGCTCC | |
| | | | AGGATGAAACCAACCTGAGGCTGGAAGCC | |
| | | | GAGAACAACCTGGCTGCCTATAGACAGGAA | |
| | | | GCAGATGAAGCCACCCTGGCCCGTCTGGAT | |
| | | | CTGGAGAGGAAGATTGAGTCGCTGGAGGA | |
| | | | GGAGATCCGGTTCTTGAGGAAGATCCACGA | |
| | | | GGAGGAGGTTCGGGAACTCCAGGAGCAGC | |
| | | | TGGCCCGACAGCAGGTCCATGTGGAGCTTG | |
| | | | ACGTGGCCAAGCCAGACCTCACCGCAGCCC | |
| | | | TGAAAGAGATCCGCACGCAGTATGAGGCA | |
| | | | ATGGCGTCCAGCAACATGCATGAAGCCGAA | |
| | | | GAGTGGTACCGCTCCAAGTTTGCAGACCTG | |
| | | | ACAGACGCTGCTGCCCGCAACGCGGAGCTG | |
| | | | CTCCGCCAGGCCAAGCACGAAGCCAACGAC | |
| | | | TACCGGCGCCAGTTGCAGTCCTTGACCTGC | |
| | | | GACCTGGAGTCTCTGCGCGGCACGAACGAG | |
| | | | TCCCTGGAGAGGCAGATGCGCGAGCAGGA | |
| | | | GGAGCGGCACGTGCGGGAGGCGGCCAGTT | |
| | | | ATCAGGAGGCGCTGGCGCGGCTGGAGGAA | |
| | | | GAGGGGCAGAGCCTCAAGGACGAGATGGC | |
| | | | CCGCCACTTGCAGGAGTACCAGGACCTGCT | |
| | | | CAATGTCAAGCTGGCCCTGGACATCGAGAT | |
| | | | CGCCACCTACAGGAAGCTGCTAGAGGGCGA | |
| | | | GGAGAACCGGATCACCATTCCCGTGCAGAC | |
| | | | CTTCTCCAACCTGCAGATTCGAGAAACCAG | |
| | | | CCTGGACACCAAGTCTGTGTCAGAAGGCCA | |
| | | | CCTCAAGAGGAACATCGTGGTGAAGACCGT | |
| | | | GGAGATGCGGGATGGAGAGGTCATTAAGG | |
| | | | AGTCCAAGCAGGAGCACAAGGATGTGATGT | |
| | | | GAGGCAGGACCCACCTGGTGGCCTCTGCCC | |
| | | | CGTCTCATGAGGGGCCCGAGCAGAAGCAG | |
| | | | GATAGTTGCTCCGCCTCTGCTGGCACATTTC | |
| | | | CCCAGACCTGAGCTCCCCACCACCCCAGCT | |
| | | | GCTCCCCTCCCTCCTCTGTCCCTAGGTCAGC | |
| | | | TTGCTGCCCTAGGCTCCGTCAGTATCAGGC | |
| | | | CTGCCAGACGGCACCCACCCAGCACCCAGC | |
| | | | AACTCCAACTAACAAGAAACTCACCCCCAA | |
| | | | GGGGCAGTCTGGAGGGGCATGGCCAGCAG | |
| | | | CTTGCGTTAGAATGAGGAGGAAGGAGAGA | |
| | | | AGGGGAGGAGGGCGGGGGGCACCTACTAC | |
| | | | ATCGCCCTCCACATCCCTGATTCCTGTTGTT | |
| | | | ATGGAAACTGTTGCCAGAGATGGAGGTTCT | |
| | | | CTCGGAGTATCTGGGAACTGTGCCTTTGAG | |
| | | | TTTCCTCAGGCTGCTGGAGGAAAACTGAGA | |
| | | | CTCAGACAGGAAAGGGAAGGCCCCACAGA | |
| | | | CAAGGTAGCCCTGGCCAGAGGCTTGTTTTG | |
| | | | TCTTTTGGTTTTTATGAGGTGGGATATCCCT | |
| | | | ATGCTGCCTAGGCTGACCTTGAACTCCTGG | |
| | | | GCTCAAGCAGTCTACCCACCTCAGCCTCCT | |
| | | | GTGTAGCTGGGATTATAGATTGGAGCCACC | |
| | | | ATGCCCAGCTCAGAGGGTTGTTCTCCTAGA | |
| | | | CTGACCCTGATCAGTCTAAGATGGGTGGGG | |
| | | | ACGTCCTGCCACCTGGGGCAGTCACCTGCC | |
| | | | CAGATCCCAGAAGGACCTCCTGAGCGATGA | |
| | | | CTCAAGTGTCTCAGTCCACCTGAGCTGCCA | |
| | | | TCCAGGGATGCCATCTGTGGGCACGCTGTG | |
| | | | GGCAGGTGGGAGCTTGATTCTCAGCACTTG | |
| | | | GGGGATCTGTTGTGTACGTGGAGAGGGATG | |
| | | | AGGTGCTGGGAGGGATAGAGGGGGGCTGC | |
| | | | CTGGCCCCCAGCTGTGGGTACAGAGAGGTC | |
| | | | AAGCCCAGGAGGACTGCCCCGTGCAGACTG | |
| | | | GAGGGGACGCTGGTAGAGATGGAGGAGGA | |
| | | | GGCAATTGGGATGGCGCTAGGCATACAAGT | |
| | | | AGGGGTTGTGGGTGACCAGTTGCACTTGGC | |
| | | | CTCTGGATTGTGGGAATTAAGGAAGTGACT | |
| | | | CATCCTCTTGAAGATGCTGAAACAGGAGAG | |
| | | | AAAGGGGATGTATCCATGGGGGCAGGGCA | |
| | | | TGACTTTGTCCCATTTCTAAAGGCCTCTTCC | |
| | | | TTGCTGTGTCATACCAGGCCGCCCCAGCCT | |
| | | | CTGAGCCCCTGGGACTGCTGCTTCTTAACCC | |
| | | | CAGTAAGCCACTGCCACACGTCTGACCCTC | |
| | | | TCCACCCCATAGTGACCGGCTGCTTTTCCCT | |
| | | | AAGCCAAGGGCCTCTTGCGGTCCCTTCTTA | |
| | | | CTCACACACAAAATGTACCCAGTATTCTAG | |
| | | | GTAGTGCCCTATTTTACAATTGTAAAACTG | |
| | | | AGGCACGAGCAAAGTGAAGACACTGGCTC | |
| | | | ATATTCCTGCAGCCTGGAGGCCGGGTGCTC | |
| | | | AGGGCTGACACGTCCACCCCAGTGCACCCA | |
| | | | CTCTGCTTTGACTGAGCAGACTGGTGAGCA | |
| | | | GACTGGTGGGATCTGTGCCCAGAGATGGGA | |
| | | | CTGGGAGGGCCCACTTCAGGGTTCTCCTCT | |
| | | | CCCCTCTAAGGCCGAAGAAGGGTCCTTCCC | |
| | | | TCTCCCCAAGACTTGGTGTCCTTTCCCTCCA | |
| | | | CTCCTTCCTGCCACCTGCTGCTGCTGCTGCT | |
| | | | GCTAATCTTCAGGGCACTGCTGCTGCCTTTA | |
| | | | GTCGCTGAGGAAAAATAAAGACAAATGCT | |
| | | | GCGCCCTTCCCCAGAGTGGACTCTGATCTG | |
| | | | TTCATGAGAGGGCGGGACTGGGGCCAAGAT | |
| | | | GTAGCCTTTGACAAGACCAACTCATTTCTTA | |
| | | | TTACTGATCATCTCTGGGGCCCATGCCCTCA | |
| | | | CCAAATTCCACCCGCAGCCAAAGAGGACAT | |
| | | | ACACCAGCTCCCTCCACTCTTTTCTTCCTTC | |
| | | | CTCTCCCTGCTACCTGCAACTCAACCAGCA | |
| | | | CAATCTTCATAGGCAAGAAAGCAAAGCAGC | |
| | | | TCAAACATGATTCAACACTGATCAGTGTTT | |
| | | | ACCACTGGATAAATCTGAGTTCACACTTTC | |
| | | | CTTCTCTGACCTAAATGTGAAGTCAGGAAA | |
| | | | CACATGTGCCCTACTTCCATCCTGAGCTCAG | |
| | | | TCCCCAATCTCCCACCAGCCTCAGGCCCCTC | |
| | | | CACTTCTCAGATCAGGTCCCAGACCTGCCC | |
| | | | ATGAAAATGGGGAGCAGGCTGTAACAGATT | |
| | | | TGTCCACATGTTCCTACCACCTGTCCCAACC | |
| | | | CAGGGTACCCACCCAGAGACATCTGGTATC | |
| | | | ATTTAACAAACACATTGAAGGACAACTGGT | |
| | | | CTTCAGAGCTGAAGAGAGCTCCTAGGGGGA | |
| | | | GAAGCTGGGACAACAGTGAAATAAGTAGC | |
| | | | AGCAGCAACGACAGAAGTGAATGGTGACA | |
| | | | AAGACTGCTGTGATGAGCAGGTAGCCTATC | |
| | | | AGGGTGAGCTCCACAGCCGAGCGAGTCTCA | |
| | | | GGATCTGAGAACGAGGCTGGGTAGTGCCCA | |
| | | | TGAGATGTCACACCCAGCCGGAAGCCAGCA | |
| | | | ACTAGCACACCCTGCCTCCAGCAATAGTAG | |
| | | | ATGCCCCGGTCATCCAGCTGGGTGAAGCGG | |
| | | | ATGTGGAGCTGGTTGCCGTGGTCAATGAAC | |
| | | | ACCCTCATGGACCTGTTGACACCCTTCAGG | |
| | | | TACTGTGTGCGGTAGAGGTGCTGGCGGTCT | |
| | | | TTGTCCCAGGCCACTGCATGCTCTGGCCGG | |
| | | | GCCCCAGGACAGGAGATGATGAGTCCATGG | |
| | | | CCCAGTCTCTGCTGGTGGAACTGAATGGGC | |
| | | | ACCTGGGGCACCCAGGGCCGGCTGCCCACT | |
| | | | TTGGACACATAGTTAATGATGGCCAGCACG | |
| | | | CCCTCCCGGATGGTCTTTGTCTTCTCACAGG | |
| | | | GTACTAAGCAGCTCCGAACCAGCACCTCAG | |
| | | | GCGTGTGGTCCCTGGCCTTGGTCCGCAGCTT | |
| | | | CCTTGGCACAGCCCTTGAGCCACAAGACAC | |
| | | | CACATCGGGCACGGCCTTGAGGTAGCGTGG | |
| | | | GGAGAGGTCTGGGCTCTGCAGGTAGCAGAG | |
| | | | GCCGATGCGCCACTGCTCCCCACGCACTCC | |
| | | | GCAGCGGTCACAGGGGGTCCATTCCCAGAA | |
| | | | GGTGGTGAAGACATGGAGGTGCCCATAGTA | |
| | | | TTCATCTGCAAAGGGCTCCTGGCCCTTGTCC | |
| | | | TGGAAAGTGGCCACCATTCCCTCACTGTTCT | |
| | | | GGATGTCCACATCGTAGGCGTAAAAGTAGT | |
| | | | CCCCCTTGCGGGTGCCGCAGAAGTACAGGC | |
| | | | CTGAGTCCTCAGACTGAGCCCTGAAAACCA | |
| | | | ACAAGCTGAACATGCGGATGCTGAAGCGG | |
| | | | GTCAGCATGTCGCTGCCCACACGTACCTGG | |
| | | | GCTGCCTCCGTCAGCACCCGCCCATCAAAG | |
| | | | TCCGTCAGCACTTTGGTGTGGCTGCTACCTA | |
| | | | GGTGCTTTTGGTAGAACCAGACTACAGCTG | |
| | | | GCACCTCTTCGGGTTTGCAGTGACAGGGAA | |
| | | | GCTCAAAGCTCATGTCGGCCAGGTAGGCTG | |
| | | | CATTTTCAAACATCAGGAAAGCAGGGCAGG | |
| | | | GGGTCCTCTGAAAAATGTTTTCCTTCTCCAC | |
| | | | AATTTCAAAGGCCTGGAGCCCCCATGCCCA | |
| | | | CAGGAGCACAGTGGTGAGGGCCAGGTGCA | |
| | | | TACCTGAAGGAGGCAGGGGTCAGAGGGGC | |
| | | | AGGGCAAAACCAGGGCATTAAAGGCTCAT | |
| | | | AGGGCTCCTAGAAAGCTCTGCTAAGCGGAA | |
| | | | GCCTCTAGATGAGGAAAGGATTATGCAGCC | |
| | | | AGGAAAAGCAGCAACAATCTGCAGAGGAA | |
| | | | GCCGCCAAGTGCAAGGCAATTTATTCCCAG | |
| | | | TGGATGTACAAGATGCCCTTCTAACATTCC | |
| | | | AGACCTGATCTCAGGGTGGGGGGGGAAAG | |
| | | | CCATTCTAGAACCTGGCCTTTACTCCCCTTT | |
| | | | CTAGAACACTGGCGCTCACCCAAGAATGGG | |
| | | | TCAAAGGAAACCGGAATGAGAAGGGCGGG | |
| | | | CCGAGGTGCTCGGGCAGGGAGATCTCTGCC | |
| | | | TCAGTGCTCCAGGCCCTGCCCTGCCAGCCT | |
| | | | GGTGGAAAAGTCTTTCATCAACCTGGGGGA | |
| | | | TGAAGGAAACCCACCCTCCTGCATATCTGG | |
| | | | CCATCCGGGAGGCTGGCTGGACCTGAGCTG | |
| | | | ATGGCTTGGGACTTTCCCAGGCCCAACCTG | |
| | | | CACAAGAACTGAGTCTCTAGGGGAAAATTC | |
| | | | AACACCTCAAATGATGTAGTATTTGATCAT | |
| | | | TTGTTGATTACATGTCCATTCATTGGTTTGG | |
| | | | GGCTATAAACATTCTTGTTAAGAGCTGTGG | |
| | | | AGATCAGTGTTTGTTTACCATAAAGATTTTG | |
| | | | CTTTTTCCCTTTTA | |
| Trans- | GFAP | N/A | AGAGCCAGAGCAGGATGGAGAGGAGACGC | 782 |
| cript | Transcript | | ATCACCTCCGCTGCTCGCCGCTCCTACGTCT | |
| variant | variant 4 | | CCTCAGGGGAGATGATGGTGGGGGGCCTGG | |
| 2 | | | CTCCTGGCCGCCGTCTGGGTCCTGGCACCC | |
| | | | GCCTCTCCCTGGCTCGAATGCCCCCTCCACT | |
| | | | CCCGACCCGGGTGGATTTCTCCCTGGCTGG | |
| | | | GGCACTCAATGCTGGCTTCAAGGAGACCCG | |
| | | | GGCCAGTGAGCGGGCAGAGATGATGGAGC | |
| | | | TCAATGACCGCTTTGCCAGCTACATCGAGA | |
| | | | AGGTTCGCTTCCTGGAACAGCAAAACAAGG | |
| | | | CGCTGGCTGCTGAGCTGAACCAGCTGCGGG | |
| | | | CCAAGGAGCCCACCAAGCTGGCAGACGTCT | |
| | | | ACCAGGCTGAGCTGCGAGAGCTGCGGCTGC | |
| | | | GGCTCGATCAACTCACCGCCAACAGCGCCC | |
| | | | GGCTGGAGGTTGAGAGGGACAATCTGGCAC | |
| | | | AGGACCTGGCCACTGTGAGGCAGAAGCTCC | |
| | | | AGGATGAAACCAACCTGAGGCTGGAAGCC | |
| | | | GAGAACAACCTGGCTGCCTATAGACAGGAA | |
| | | | GCAGATGAAGCCACCCTGGCCCGTCTGGAT | |
| | | | CTGGAGAGGAAGATTGAGTCGCTGGAGGA | |
| | | | GGAGATCCGGTTCTTGAGGAAGATCCACGA | |
| | | | GGAGGAGGTTCGGGAACTCCAGGAGCAGC | |
| | | | TGGCCCGACAGCAGGTCCATGTGGAGCTTG | |
| | | | ACGTGGCCAAGCCAGACCTCACCGCAGCCC | |
| | | | TGAAAGAGATCCGCACGCAGTATGAGGCA | |
| | | | ATGGCGTCCAGCAACATGCATGAAGCCGAA | |
| | | | GAGTGGTACCGCTCCAAGTTTGCAGACCTG | |
| | | | ACAGACGCTGCTGCCCGCAACGCGGAGCTG | |
| | | | CTCCGCCAGGCCAAGCACGAAGCCAACGAC | |
| | | | TACCGGCGCCAGTTGCAGTCCTTGACCTGC | |
| | | | GACCTGGAGTCTCTGCGCGGCACGAACGAG | |
| | | | TCCCTGGAGAGGCAGATGCGCGAGCAGGA | |
| | | | GGAGCGGCACGTGCGGGAGGCGGCCAGTT | |
| | | | ATCAGGAGGCGCTGGCGCGGCTGGAGGAA | |
| | | | GAGGGGCAGAGCCTCAAGGACGAGATGGC | |
| | | | CCGCCACTTGCAGGAGTACCAGGACCTGCT | |
| | | | CAATGTCAAGCTGGCCCTGGACATCGAGAT | |
| | | | CGCCACCTACAGGAAGCTGCTAGAGGGCGA | |
| | | | GGAGAACCGGATCACCATTCCCGTGCAGAC | |
| | | | CTTCTCCAACCTGCAGATTCGAGGGGGCAA | |
| | | | AAGCACCAAAGACGGGGAAAATCACAAGG | |
| | | | TCACAAGATATCTCAAAAGCCTCACAATAC | |
| | | | GAGTTATACCAATACAGGCTCACCAGATTG | |
| | | | TAAATGGAACGCCGCCGGCTCGCGGTTAGC | |
| | | | TGCCTGCCTCTCAGACACGGCGCTTTGCCC | |
| | | | AGCTTGACAGGGAGTGAGCCTCACCCACCC | |
| | | | CATCCTCCCAATCCCCCTGAGTTCCCTCTTC | |
| | | | CCAGGCTTCCCCTAAAGGGCCTGGACTGCG | |
| | | | TCATTTTCCCAGGAACTGCAGTGCCCAGCC | |
| | | | CAGGACGTGGTACAGAGTAACTGTACATTA | |
| | | | AACTGGCAGAGCTTGTTAGTGGTAAAGGTG | |
| | | | GTGAGTCCTTGGGTGCGCAGTGGAGCTGCT | |
| | | | CTGGGGCCTCTGAGCAAGCAGCAGCCTCTG | |
| | | | TCTCACCTCTTCCTGTCACTGGGAGGGCCCC | |
| | | | TTGGGTCTCGCTGTGCCTGGACGCCAGGCT | |
| | | | CTCTGCTTTATTCTTTCATCCCTGAGGCTCC | |
| | | | ATCGCTCAGCTCAGTGCTGACTCAGTTCAG | |
| | | | AGGATTCTTCCCTCAGGACCGCAGCTCTTG | |
| | | | CAGTGAATAAAGTTTTATGTTCCCTGCTCTT | |
| | | | AATGTTAAA | |
| Trans- | GFAP | N/A | AGAGCCAGAGCAGGATGGAGAGGAGACGC | 783 |
| cript | Transcript | | ATCACCTCCGCTGCTCGCCGCTCCTACGTCT | |
| variant | variant 3 | | CCTCAGGGGAGATGATGGTGGGGGGCCTGG | |
| 3 | | | CTCCTGGCCGCCGTCTGGGTCCTGGCACCC | |
| | | | GCCTCTCCCTGGCTCGAATGCCCCCTCCACT | |
| | | | CCCGACCCGGGTGGATTTCTCCCTGGCTGG | |
| | | | GGCACTCAATGCTGGCTTCAAGGAGACCCG | |
| | | | GGCCAGTGAGCGGGCAGAGATGATGGAGC | |
| | | | TCAATGACCGCTTTGCCAGCTACATCGAGA | |
| | | | AGGTTCGCTTCCTGGAACAGCAAAACAAGG | |
| | | | CGCTGGCTGCTGAGCTGAACCAGCTGCGGG | |
| | | | CCAAGGAGCCCACCAAGCTGGCAGACGTCT | |
| | | | ACCAGGCTGAGCTGCGAGAGCTGCGGCTGC | |
| | | | GGCTCGATCAACTCACCGCCAACAGCGCCC | |
| | | | GGCTGGAGGTTGAGAGGGACAATCTGGCAC | |
| | | | AGGACCTGGCCACTGTGAGGCAGAAGCTCC | |
| | | | AGGATGAAACCAACCTGAGGCTGGAAGCC | |
| | | | GAGAACAACCTGGCTGCCTATAGACAGGAA | |
| | | | GCAGATGAAGCCACCCTGGCCCGTCTGGAT | |
| | | | CTGGAGAGGAAGATTGAGTCGCTGGAGGA | |
| | | | GGAGATCCGGTTCTTGAGGAAGATCCACGA | |
| | | | GGAGGAGGTTCGGGAACTCCAGGAGCAGC | |
| | | | TGGCCCGACAGCAGGTCCATGTGGAGCTTG | |
| | | | ACGTGGCCAAGCCAGACCTCACCGCAGCCC | |
| | | | TGAAAGAGATCCGCACGCAGTATGAGGCA | |
| | | | ATGGCGTCCAGCAACATGCATGAAGCCGAA | |
| | | | GAGTGGTACCGCTCCAAGTTTGCAGACCTG | |
| | | | ACAGACGCTGCTGCCCGCAACGCGGAGCTG | |
| | | | CTCCGCCAGGCCAAGCACGAAGCCAACGAC | |
| | | | TACCGGCGCCAGTTGCAGTCCTTGACCTGC | |
| | | | GACCTGGAGTCTCTGCGCGGCACGAACGAG | |
| | | | TCCCTGGAGAGGCAGATGCGCGAGCAGGA | |
| | | | GGAGCGGCACGTGCGGGAGGCGGCCAGTT | |
| | | | ATCAGGAGGCGCTGGCGCGGCTGGAGGAA | |
| | | | GAGGGGCAGAGCCTCAAGGACGAGATGGC | |
| | | | CCGCCACTTGCAGGAGTACCAGGACCTGCT | |
| | | | CAATGTCAAGCTGGCCCTGGACATCGAGAT | |
| | | | CGCCACCTACAGGAAGCTGCTAGAGGGCGA | |
| | | | GGAGAACCGGATCACCATTCCCGTGCAGAC | |
| | | | CTTCTCCAACCTGCAGATTCGAGGTCAGTA | |
| | | | CAGCAGGGCCTCGTGGGAAGGGCACTGGA | |
| | | | GTCCTGCCCCCTCCTCCAGGGCCTGTAGGTT | |
| | | | GCTCCAGACTGGGACTGAGGATCAGGGCAA | |
| | | | AGGGATCCAGCTCTCCCTGGGGGCCTTCGT | |
| | | | GACACTGCAGCGCTCCTAGCCAGAGCCTAT | |
| | | | CATACCAGGGTACTTCTAGGTGGGGCTTGC | |
| | | | AGCTGCCCCTGTCCTGCTAGGCCCTGGTCCC | |
| | | | TCTTCCCCTCCCTGCACCCCATTCGACAGCA | |
| | | | GAACTGGGTGAGAGCTTGACATCTGCCCTG | |
| | | | TCTGCAGATCCCTGAGCAAGCACTGCCCTT | |
| | | | CTGAGTGTTTTCTGTTTTTGTTTTTTTAACTG | |
| | | | CTTGTCACTACAGGGGGCAAAAGCACCAAA | |
| | | | GACGGGGAAAATCACAAGGTCACAAGATA | |
| | | | TCTCAAAAGCCTCACAATACGAGTTATACC | |
| | | | AATACAGGCTCACCAGATTGTAAATGGAAC | |
| | | | GCCGCCGGCTCGCGGTTAGCTGCCTGCCTC | |
| | | | TCAGACACGGCGCTTTGCCCAGCTTGACAG | |
| | | | GGAGTGAGCCTCACCCACCCCATCCTCCCA | |
| | | | ATCCCCCTGAGTTCCCTCTTCCCAGGCTTCC | |
| | | | CCTAAAGGGCCTGGACTGCGTCATTTTCCC | |
| | | | AGGAACTGCAGTGCCCAGCCCAGGACGTGG | |
| | | | TACAGAGTAACTGTACATTAAACTGGCAGA | |
| | | | GCTTGTTAGTGGTAAAGGTGGTGAGTCCTT | |
| | | | GGGTGCGCAGTGGAGCTGCTCTGGGGCCTC | |
| | | | TGAGCAAGCAGCAGCCTCTGTCTCACCTCT | |
| | | | TCCTGTCACTGGGAGGGCCCCTTGGGTCTC | |
| | | | GCTGTGCCTGGACGCCAGGCTCTCTGCTTTA | |
| | | | TTCTTTCATCCCTGAGGCTCCATCGCTCAGC | |
| | | | TCAGTGCTGACTCAGTTCAGAGGATTCTTCC | |
| | | | CTCAGGACCGCAGCTCTTGCAGTGAATAAA | |
| | | | GTTTTATGTTCCCTGCTCTTAATGTTAAA | |
| Trans- | GFAP | N/A | AGAGCCAGAGCAGGATGGAGAGGAGACGC | 784 |
| cript | Transcript | | ATCACCTCCGCTGCTCGCCGCTCCTACGTCT | |
| variant | variant 4 | | CCTCAGGGGAGATGATGGTGGGGGGCCTGG | |
| 4 | | | CTCCTGGCCGCCGTCTGGGTCCTGGCACCC | |
| | | | GCCTCTCCCTGGCTCGAATGCCCCCTCCACT | |
| | | | CCCGACCCGGGTGGATTTCTCCCTGGCTGG | |
| | | | GGCACTCAATGCTGGCTTCAAGGAGACCCG | |
| | | | GGCCAGTGAGCGGGCAGAGATGATGGAGC | |
| | | | TCAATGACCGCTTTGCCAGCTACATCGAGA | |
| | | | AGGTTCGCTTCCTGGAACAGCAAAACAAGG | |
| | | | CGCTGGCTGCTGAGCTGAACCAGCTGCGGG | |
| | | | CCAAGGAGCCCACCAAGCTGGCAGACGTCT | |
| | | | ACCAGGCTGAGCTGCGAGAGCTGCGGCTGC | |
| | | | GGCTCGATCAACTCACCGCCAACAGCGCCC | |
| | | | GGCTGGAGGTTGAGAGGGACAATCTGGCAC | |
| | | | AGGACCTGGCCACTGTGAGGCAGAAGCTCC | |
| | | | AGGATGAAACCAACCTGAGGCTGGAAGCC | |
| | | | GAGAACAACCTGGCTGCCTATAGACAGGAA | |
| | | | GCAGATGAAGCCACCCTGGCCCGTCTGGAT | |
| | | | CTGGAGAGGAAGATTGAGTCGCTGGAGGA | |
| | | | GGAGATCCGGTTCTTGAGGAAGATCCACGA | |
| | | | GGAGGAGGTTCGGGAACTCCAGGAGCAGC | |
| | | | TGGCCCGACAGCAGGTCCATGTGGAGCTTG | |
| | | | ACGTGGCCAAGCCAGACCTCACCGCAGCCC | |
| | | | TGAAAGAGATCCGCACGCAGTATGAGGCA | |
| | | | ATGGCGTCCAGCAACATGCATGAAGCCGAA | |
| | | | GAGTGGTACCGCTCCAAGTTTGCAGACCTG | |
| | | | ACAGACGCTGCTGCCCGCAACGCGGAGCTG | |
| | | | CTCCGCCAGGCCAAGCACGAAGCCAACGAC | |
| | | | TACCGGCGCCAGTTGCAGTCCTTGACCTGC | |
| | | | GACCTGGAGTCTCTGCGCGGCACGAACGAG | |
| | | | TCCCTGGAGAGGCAGATGCGCGAGCAGGA | |
| | | | GGAGCGGCACGTGCGGGAGGCGGCCAGTT | |
| | | | ATCAGGAGGCGCTGGCGCGGCTGGAGGAA | |
| | | | GAGGGGCAGAGCCTCAAGGACGAGATGGC | |
| | | | CCGCCACTTGCAGGAGTACCAGGACCTGCT | |
| | | | CAATGTCAAGCTGGCCCTGGACATCGAGAT | |
| | | | CGCCACCTACAGGAAGCTGCTAGAGGGCGA | |
| | | | GGAGAACCGGATCACCATTCCCGTGCAGAC | |
| | | | CTTCTCCAACCTGCAGATTCGAGGGGGCAA | |
| | | | AAGCACCAAAGACGGGGAAAATCACAAGG | |
| | | | TCACAAGATATCTCAAAAGCCTCACAATAC | |
| | | | GAGTTATACCAATACAGGCTCACCAGATTG | |
| | | | TAAATGGAACGCCGCCGGCTCGCGAAACCA | |
| | | | GCCTGGACACCAAGTCTGTGTCAGAAGGCC | |
| | | | ACCTCAAGAGGAACATCGTGGTGAAGACCG | |
| | | | TGGAGATGCGGGATGGAGAGGTCATTAAG | |
| | | | GAGTCCAAGCAGGAGCACAAGGATGTGAT | |
| | | | GTGAGGCAGGACCCACCTGGTGGCCTCTGC | |
| | | | CCCGTCTCATGAGGGGCCCGAGCAGAAGCA | |
| | | | GGATAGTTGCTCCGCCTCTGCTGGCACATTT | |
| | | | CCCCAGACCTGAGCTCCCCACCACCCCAGC | |
| | | | TGCTCCCCTCCCTCCTCTGTCCCTAGGTCAG | |
| | | | CTTGCTGCCCTAGGCTCCGTCAGTATCAGG | |
| | | | CCTGCCAGACGGCACCCACCCAGCACCCAG | |
| | | | CAACTCCAACTAACAAGAAACTCACCCCCA | |
| | | | AGGGGCAGTCTGGAGGGGCATGGCCAGCA | |
| | | | GCTTGCGTTAGAATGAGGAGGAAGGAGAG | |
| | | | AAGGGGAGGAGGGCGGGGGGCACCTACTA | |
| | | | CATCGCCCTCCACATCCCTGATTCCTGTTGT | |
| | | | TATGGAAACTGTTGCCAGAGATGGAGGTTC | |
| | | | TCTCGGAGTATCTGGGAACTGTGCCTTTGA | |
| | | | GTTTCCTCAGGCTGCTGGAGGAAAACTGAG | |
| | | | ACTCAGACAGGAAAGGGAAGGCCCCACAG | |
| | | | ACAAGGTAGCCCTGGCCAGAGGCTTGTTTT | |
| | | | GTCTTTTGGTTTTTATGAGGTGGGATATCCC | |
| | | | TATGCTGCCTAGGCTGACCTTGAACTCCTG | |
| | | | GGCTCAAGCAGTCTACCCACCTCAGCCTCC | |
| | | | TGTGTAGCTGGGATTATAGATTGGAGCCAC | |
| | | | CATGCCCAGCTCAGAGGGTTGTTCTCCTAG | |
| | | | ACTGACCCTGATCAGTCTAAGATGGGTGGG | |
| | | | GACGTCCTGCCACCTGGGGCAGTCACCTGC | |
| | | | CCAGATCCCAGAAGGACCTCCTGAGCGATG | |
| | | | ACTCAAGTGTCTCAGTCCACCTGAGCTGCC | |
| | | | ATCCAGGGATGCCATCTGTGGGCACGCTGT | |
| | | | GGGCAGGTGGGAGCTTGATTCTCAGCACTT | |
| | | | GGGGGATCTGTTGTGTACGTGGAGAGGGAT | |
| | | | GAGGTGCTGGGAGGGATAGAGGGGGGCTG | |
| | | | CCTGGCCCCCAGCTGTGGGTACAGAGAGGT | |
| | | | CAAGCCCAGGAGGACTGCCCCGTGCAGACT | |
| | | | GGAGGGGACGCTGGTAGAGATGGAGGAGG | |
| | | | AGGCAATTGGGATGGCGCTAGGCATACAAG | |
| | | | TAGGGGTTGTGGGTGACCAGTTGCACTTGG | |
| | | | CCTCTGGATTGTGGGAATTAAGGAAGTGAC | |
| | | | TCATCCTCTTGAAGATGCTGAAACAGGAGA | |
| | | | GAAAGGGGATGTATCCATGGGGGCAGGGC | |
| | | | ATGACTTTGTCCCATTTCTAAAGGCCTCTTC | |
| | | | CTTGCTGTGTCATACCAGGCCGCCCCAGCC | |
| | | | TCTGAGCCCCTGGGACTGCTGCTTCTTAACC | |
| | | | CCAGTAAGCCACTGCCACACGTCTGACCCT | |
| | | | CTCCACCCCATAGTGACCGGCTGCTTTTCCC | |
| | | | TAAGCCAAGGGCCTCTTGCGGTCCCTTCTTA | |
| | | | CTCACACACAAAATGTACCCAGTATTCTAG | |
| | | | GTAGTGCCCTATTTTACAATTGTAAAACTG | |
| | | | AGGCACGAGCAAAGTGAAGACACTGGCTC | |
| | | | ATATTCCTGCAGCCTGGAGGCCGGGTGCTC | |
| | | | AGGGCTGACACGTCCACCCCAGTGCACCCA | |
| | | | CTCTGCTTTGACTGAGCAGACTGGTGAGCA | |
| | | | GACTGGTGGGATCTGTGCCCAGAGATGGGA | |
| | | | CTGGGAGGGCCCACTTCAGGGTTCTCCTCT | |
| | | | CCCCTCTAAGGCCGAAGAAGGGTCCTTCCC | |
| | | | TCTCCCCAAGACTTGGTGTCCTTTCCCTCCA | |
| | | | CTCCTTCCTGCCACCTGCTGCTGCTGCTGCT | |
| | | | GCTAATCTTCAGGGCACTGCTGCTGCCTTTA | |
| | | | GTCGCTGAGGAAAAATAAAGACAAATGCT | |
| | | | GCGCCCTTCCCCA | |
| Stem | N/A | N/A | GCAGCCGAAAGGCUGC | 785 |
| Loop | | | | |
| UGT8- | 19 mer sense | N/A | CAAUUAUGUUUGAAAGCCA | 786 |
| 277 | strand | | | |
| UGT8- | 19 mer sense | N/A | AAUUAUGUUUGAAAGCCAU | 787 |
| 278 | strand | | | |
| UGT8- | 19 mer sense | N/A | UUGACAUACUGGAUCACUA | 788 |
| 505 | strand | | | |
| UGT8- | 19 mer sense | N/A | ACAUACUGGAUCACUAUAC | 789 |
| 508 | strand | | | |
| UGT8- | 19 mer sense | N/A | CAUACUGGAUCACUAUACU | 790 |
| 509 | strand | | | |
| UGT8- | 19 mer sense | N/A | CUGGAUCACUAUACUAAGA | 791 |
| 513 | strand | | | |
| UGT8- | 19 mer sense | N/A | AUAUGUGUGGAUUUGUGAU | 792 |
| 616 | strand | | | |
| UGT8- | 19 mer sense | N/A | AGGAUAAUGCAGAAGUACA | 793 |
| 843 | strand | | | |
| UGT8- | 19 mer sense | N/A | AGCAUAGCACAGUUAAUGG | 794 |
| 1726 | strand | | | |
| UGT8- | 19 mer | N/A | UGGCUUUCAAACAUAAUUG | 795 |
| 277 | antisense | | | |
| | strand | | | |
| UGT8- | 19 mer | N/A | AUGGCUUUCAAACAUAAUU | 796 |
| 278 | antisense | | | |
| | strand | | | |
| UGT8- | 19 mer | N/A | UAGUGAUCCAGUAUGUCAA | 797 |
| 505 | antisense | | | |
| | strand | | | |
| UGT8- | 19 mer | N/A | GUAUAGUGAUCCAGUAUGU | 798 |
| 508 | antisense | | | |
| | strand | | | |
| UGT8- | 19 mer | N/A | AGUAUAGUGAUCCAGUAUG | 799 |
| 509 | antisense | | | |
| | strand | | | |
| UGT8- | 19 mer | N/A | UCUUAGUAUAGUGAUCCAG | 800 |
| 513 | antisense | | | |
| | strand | | | |
| UGT8- | 19 mer | N/A | AUCACAAAUCCACACAUAU | 801 |
| 616 | antisense | | | |
| | strand | | | |
| UGT8- | 19 mer | N/A | UGUACUUCUGCAUUAUCCU | 802 |
| 843 | antisense | | | |
| | strand | | | |
| UGT8- | 19 mer | N/A | CCAUUAACUGUGCUAUGCU | 803 |
| 1726 | antisense | | | |
| | strand | | | |
| UGT8- | 36 mer sense | N/A | CAAUUAUGUUUGAAAGCCAAGCAGCCGAA | 804 |
| 277 | strand | | AGGCUGC | |
| UGT8- | 36 mer sense | N/A | AAUUAUGUUUGAAAGCCAUAGCAGCCGAA | 805 |
| 278 | strand | | AGGCUGC | |
| UGT8- | 36 mer sense | N/A | UUGACAUACUGGAUCACUAAGCAGCCGAA | 806 |
| 505 | strand | | AGGCUGC | |
| UGT8- | 36 mer sense | N/A | ACAUACUGGAUCACUAUACAGCAGCCGAA | 807 |
| 508 | strand | | AGGCUGC | |
| UGT8- | 36 mer sense | N/A | CAUACUGGAUCACUAUACUAGCAGCCGAA | 808 |
| 509 | strand | | AGGCUGC | |
| UGT8- | 36 mer sense | N/A | CUGGAUCACUAUACUAAGAAGCAGCCGAA | 809 |
| 513 | strand | | AGGCUGC | |
| UGT8- | 36 mer sense | N/A | AUAUGUGUGGAUUUGUGAUAGCAGCCGAA | 810 |
| 616 | strand | | AGGCUGC | |
| UGT8- | 36 mer sense | N/A | AGGAUAAUGCAGAAGUACAAGCAGCCGAA | 811 |
| 843 | strand | | AGGCUGC | |
| UGT8- | 36 mer sense | N/A | AGCAUAGCACAGUUAAUGGAGCAGCCGAA | 812 |
| 1726 | strand | | AGGCUGC | |
| UGT8- | 22 mer | N/A | UUGGCUUUCAAACAUAAUUGGG | 813 |
| 277 | antisense | | | |
| | strand | | | |
| UGT8- | 22 mer | N/A | UAUGGCUUUCAAACAUAAUUGG | 814 |
| 278 | antisense | | | |
| | strand | | | |
| UGT8- | 22 mer | N/A | UUAGUGAUCCAGUAUGUCAAGG | 815 |
| 505 | antisense | | | |
| | strand | | | |
| UGT8- | 22 mer | N/A | UGUAUAGUGAUCCAGUAUGUGG | 816 |
| 508 | antisense | | | |
| | strand | | | |
| UGT8- | 22 mer | N/A | UAGUAUAGUGAUCCAGUAUGGG | 817 |
| 509 | antisense | | | |
| | strand | | | |
| UGT8- | 22 mer | N/A | UUCUUAGUAUAGUGAUCCAGGG | 818 |
| 513 | antisense | | | |
| | strand | | | |
| UGT8- | 22 mer | N/A | UAUCACAAAUCCACACAUAUGG | 819 |
| 616 | antisense | | | |
| | strand | | | |
| UGT8- | 22 mer | N/A | UUGUACUUCUGCAUUAUCCUGG | 820 |
| 843 | antisense | | | |
| | strand | | | |
| UGT8- | 22 mer | N/A | UCCAUUAACUGUGCUAUGCUGG | 821 |
| 1726 | antisense | | | |
| | strand | | | |
| UGT8- | Modified | Tetraloop | [mCs][mA][mA][mU][mU][mA][mU][fG][fU][fU] | 822 |
| 277 | 36 mer sense | GalNAc | [fU][mG][mA][mA][mA][mG][mC][mC][mA][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mAs][mA][mU][mU][mA][mU][mG][fU][fU][fU] | 823 |
| 278 | 36 mer sense | GalNAc | [fG][mA][mA][mA][mG][mC][mC][mA][mU][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mUs][mU][mG][mA][mC][mA][mU][fA][fC][fU] | 824 |
| 505 | 36 mer sense | GalNAc | [fG][mG][mA][mU][mC][mA][mC][mU][mA][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mAs][mC][mA][mU][mA][mC][mU][fG][fG][fA] | 825 |
| 508 | 36 mer sense | GalNAc | [fU][mC][mA][mC][mU][mA][mU][mA][mC][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mCs][mA][mU][mA][mC][mU][mG][fG][fA][fU] | 826 |
| 509 | 36 mer sense | GalNAc | [fC][mA][mC][mU][mA][mU][mA][mC][mU][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mCs][mU][mG][mG][mA][mU][mC][fA][fC][fU] | 827 |
| 513 | 36 mer sense | GalNAc | [fA][mU][mA][mC][mU][mA][mA][mG][mA][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mAs][mU][mA][mU][mG][mU][mG][fU][fG][fG] | 828 |
| 616 | 36 mer sense | GalNAc | [fA][mU][mU][mU][mG][mU][mG][mA][mU][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mAs][mG][mG][mA][mU][mA][mA][fU][fG][fC] | 829 |
| 843 | 36 mer sense | GalNAc | [fA][mG][mA][mA][mG][mU][mA][mC][mA][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | Tetraloop | [mAs][mG][mC][mA][mU][mA][mG][fC][fA][fC] | 830 |
| 1726 | 36 mer sense | GalNAc | [fA][mG][mU][mU][mA][mA][mU][mG][mG][mA] | |
| | strand | | [mG][mC][mA][mG][mC][mC][mG][ademA- | |
| | | | GalNAc][ademA-GalNAc][ademA- | |
| | | | GalNAc][mG][mG][mC][mU][mG][mC] | |
| UGT8- | Modified | N/A | [MePhosphonate-4O- | 831 |
| 277 | 22 mer | | mUs][fUs][fG][fG][fC][mU][fU][mU][mC][fA] | |
| | antisense | | [mA][mA][mC][fA][mU][mA][mA][mU][mU][mGs] | |
| | strand | | [mGs][mG] | |
| UGT8- | Modified | N/A | [MePhosphonate-4O- | 832 |
| 278 | 22 mer | | mUs][fAs][fU][fG][fG][mC][fU][mU][mU][fC] | |
| | antisense | | [mA][mA][mA][fC][mA][mU][mA][mA][mU][mUs] | |
| | strand | | [mGs][mG] | |
| UGT8- | Modified | N/A | [MePhosphonate-4O- | 833 |
| 505 | 22 mer | | mUs][fUs][fA][fG][fU][mG][fA][mU][mC][fC] | |
| | antisense | | [mA][mG][mU][fA][mU][mG][mU][mC][mA][mAs] | |
| | strand | | [mGs][mG] | |
| UGT8- | Modified | N/A | [MePhosphonate-4O- | 834 |
| 508 | 22 mer | | mUs][fGs][fU][fA][fU][mA][fG][mU][mG][fA] | |
| | antisense | | [mU][mC][mC][fA][mG][mU][mA][mU][mG][mUs] | |
| | strand | | [mGs][mG] | |
| UGT8- | Modified | N/A | [MePhosphonate-4O- | 835 |
| 509 | 22 mer | | mUs][fAs][fG][fU][fA][mU][fA][mG][mU][fG] | |
| | antisense | | [mA][mU][mC][fC][mA][mG][mU][mA][mU][mGs] | |
| | strand | | [mGs][mG] | |
| UGT8- | Modified | N/A | [MePhosphonate-4O- | 836 |
| 513 | 22 mer | | mUs][fUs][fC][fU][fU][mA][fG][mU][mA][fU] | |
| | antisense | | [mA][mG][mU][fG][mA][mU][mC][mC][mA][mGs] | |
| | strand | | [mGs][mG] | |
| UGT8- | Modified | N/A | [MePhosphonate-4O- | 837 |
| 616 | 22 mer | | mUs][fAs][fU][fC][fA][mC][fA][mA][mA][fU] | |
| | antisense | | [mC][mC][mA][fC][mA][mC][mA][mU][mA][mUs] | |
| | strand | | [mGs][mG] | |
| UGT8- | Modified | N/A | [MePhosphonate-4O- | 838 |
| 843 | 22 mer | | mUs][fUs][fG][fU][fA][mC][fU][mU][mC][fU] | |
| | antisense | | [mG][mC][mA][fU][mU][mA][mU][mC][mC][mUs] | |
| | strand | | [mGs][mG] | |
| UGT8- | Modified | N/A | [MePhosphonate-4O- | 839 |
| 1726 | 22 mer | | mUs][fCs][fC][fA][fU][mU][fA][mA] | |
| | antisense | | [mC][fU][mG][mU][mG][fC][mU][mA][mU][mG] | |
| | strand | | [mC][mUs][mGs][mG] | |
| UGT8- | Modified | N/A | [MePhosphonate-4O- | 840 |
| 277 | 22 mer | (three | mUs][fUs][fGs][fG][fC][mU][fU][mU][mC][fA] | |
| | antisense | phosphor- | [mA][mA][mC][fA][mU][mA][mA][mU][mU][mGs] | |
| | strand | othioate | [mGs][mG] | |
| | | linkages) | | |
| UGT8- | Modified | Tetraloop | [mCs][mA][mA][mU][mU][mA][mU][fG][fU][fU] | 841 |
| 277 | 36 mer sense | No | [fU][mG][mA][mA][mA][mG][mC][mC][mA][mA] | |
| | strand | Ligand | [mG][mC][mA][mG][mC][mC][mG][mA][mA] | |
| | | | [mA][mG][mG][mC][mU][mG][mC] | |
| UGT8- | Modified | Tetraloop | [mCs][ademA- | 842 |
| 277 | 36 mer sense | P2 | C16][mA][mU][mU][mA][mU][fG][fU][fU][fU] | |
| | strand | C16 | [mG][mA][mA][mA][mG][mC][mC][mA][mA][mG] | |
| | | | [mC][mA][mG][mC][mC][mG][mA][mA][mA][mG] | |
| | | | [mG][mC][mU][mG][mC] | |
| UGT8- | Modified | Tetraloop | [mCs][mA][ademA- | 843 |
| 277 | 36 mer sense | P3 | C16][mU][mU][mA][mU][fG][fU][fU][fU][mG] | |
| | strand | C16 | [mA][mA][mA][mG][mC][mC][mA][mA][mG][mC] | |
| | | | [mA][mG][mC][mC][mG][mA][mA][mA][mG] | |
| | | | [mG][mC][mU][mG][mC] | |
| UGT8- | Modified | Tetraloop | [mCs][mA][mA][mU][mU][ademA- | 844 |
| 277 | 36 mer sense | P6 | C16][mU][fG][fU][fU][fU][mG][mA][mA][mA] | |
| | strand | C16 | [mG][mC][mC][mA][mA][mG][mC][mA][mG][mC] | |
| | | | [mC][mG][mA][mA][mA][mG][mG][mC][mU] | |
| | | | [mG][mC] | |
| UGT8- | Modified | Tetraloop | [mCs][mA][mA][mU][mU][mA][mU][fG][fU][fU] | 845 |
| 277 | 36 mer sense | P13 | [fU][mG][ademA- | |
| | strand | C16 | C16][mA][mA][mG][mC][mC][mA][mA][mG] | |
| | | | [mC][mA][mG][mC][mC][mG][mA][mA][mA][mG] | |
| | | | [mG][mC][mU][mG][mC] | |
| UGT8- | Modified | Tetraloop | [mCs][mA][mA][mU][mU][mA][mU][fG][fU][fU] | 846 |
| 277 | 36 mer sense | P14 | [fU][mG][mA][ademA- | |
| | strand | C16 | C16][mA][mG][mC][mC][mA][mA][mG][mC] | |
| | | | [mA][mG][mC][mC][mG][mA][mA][mA][mG][mG] | |
| | | | [mC][mU][mG][mC] | |
| UGT8- | Modified | Tetraloop | [mCs][mA][mA][mU][mU][mA][mU][fG][fU][fU] | 847 |
| 277 | 36 mer sense | P15 | [fU][mG][mA][mA][ademA- | |
| | strand | C16 | C16][mG][mC][mC][mA][mA][mG][mC][mA] | |
| | | | [mG][mC][mC][mG][mA][mA][mA][mG][mG][mC] | |
| | | | [mU][mG][mC] | |
| UGT8- | Modified | Tetraloop | [mCs][mA][mA][mU][mU][mA][mU][fG][fU][fU] | 848 |
| 277 | 36 mer sense | P19 | [fU][mG][mA][mA][mA][mG][mC][mC][ademA- | |
| | strand | C16 | C16][mA][mG][mC][mA][mG][mC][mC][mG] | |
| | | | [mA][mA][mA][mG][mG][mC][mU][mG][mC] | |
| UGT8- | Modified | Tetraloop | [mCs][mA][mA][mU][mU][mA][mU][fG][fU][fU] | 849 |
| 277 | 36 mer sense | P20 | [fU][mG][mA][mA][mA][mG][mC][mC][mA] | |
| | strand | C16 | [ademA- | |
| | | | C16][mG][mC][mA][mG][mC][mC][mG][mA] | |
| | | | [mA][mA][mG][mG][mC][mU][mG][mC] | |
| UGT8- | Modified | Tetraloop | [mCs][mA][mA][mU][mU][mA][mU][fG][fU][fU] | 850 |
| 277 | 36 mer sense | P23 | [fU][mG][mA][mA][mA][mG][mC][mC][mA] | |
| | strand | C16 | [mA][mG][mC][ademA- | |
| | | | C16][mG][mC][mC][mG][mA][mA][mA][mG] | |
| | | | [mG][mC][mU][mG][mC] | |
| UGT8- | Modified | Tetraloop | [mCs][mA][mA][mU][mU][mA][mU][fG][fU][fU] | 851 |
| 277 | 36 mer sense | P28 | [fU][mG][mA][mA][mA][mG][mC][mC][mA][mA] | |
| | strand | C16 | [mG][mC][mA][mG][mC][mC][mG][ademA- | |
| | | | C16][mA][mA][mG][mG][mC][mU][mG][mC] | |
| UGT8- | Modified | Tetraloop | [mCs][mA][mA][mU][mU][mA][mU][fG][fU][fU] | 852 |
| 277 | 36 mer sense | P29 | [fU][mG][mA][mA][mA][mG][mC][mC][mA][mA] | |
| | strand | C16 | [mG][mC][mA][mG][mC][mC][mG][mA][ademA- | |
| | | | C16][mA][mG][mG][mC][mU][mG][mC] | |
| UGT8- | Modified | Tetraloop | [mCs][mA][mA][mU][mU][mA][mU][fG][fU][fU] | 853 |
| 277 | 36 mer sense | P30 | [fU][mG][mA][mA][mA][mG][mC][mC][mA][mA] | |
| | strand | C16 | [mG][mC][mA][mG][mC][mC][mG][mA][mA] | |
| | | | [ademA-C16][mG][mG][mC][mU][mG][mC] | |
| UGT8- | Modified | Tetraloop | [ademCs- | 854 |
| 277 | 36 mer sense | P1 | C16][mA][mA][mU][mU][mA][mU][fG][fU][fU] | |
| | strand | AdemC | [fU][mG][mA][mA][mA][mG][mC][mC][mA][mA] | |
| | | C16 | [mG][mC][mA][mG][mC][mC][mG][mA][mA][mA] | |
| | | | [mG][mG][mC][mU][mG][mC] | |
| UGT8- | Modified | Tetraloop | [ademAs- | 855 |
| 277 | 36 mer sense | P1 | C16][mA][mA][mU][mU][mA][mU][fG][fU][fU] | |
| | strand | AdemA | [fU][mG][mA][mA][mA][mG][mC][mC][mA][mA] | |
| | | C16 | [mG][mC][mA][mG][mC][mC][mG][mA][mA][mA] | |
| | | mismatch | [mG][mG][mC][mU][mG][mC] | |
| UGT8- | Unmodified | N/A | CAAUUAUGUUUGAAAGCCAA | 856 |
| 277 | 20 mer sense | | | |
| | strand | | | |
| UGT8- | Modified | Blunt- | [mCs][mA][mA][mU][mU][mA][mU][fG][fU][fU] | 857 |
| 277 | 20 mer sense | End No | [fU][mG][mA][mA][mA][mG][mC][mCs][mAs] | |
| | strand | Ligand | [mA] | |
| UGT8- | Modified | Blunt- | [mCs][ademA- | 858 |
| 277 | 20 mer sense | End P2 | C16][mA][mU][mU][mA][mU][fG][fU][fU][fU] | |
| | strand | C16 | [mG][mA][mA][mA][mG][mC][mCs][mAs][mA] | |
| UGT8- | Modified | Blunt- | [mCs][mA][ademA- | 859 |
| 277 | 20 mer sense | End P3 | C16][mU][mU][mA][mU][fG][fU][fU][fU][mG] | |
| | strand | C16 | [mA][mA][mA][mG][mC][mCs][mAs][mA] | |
| UGT8- | Modified | Blunt- | [mCs][mA][mA][mU][mU][ademA- | 860 |
| 277 | 20 mer sense | End P6 | C16][mU][fG][fU][fU][fU][mG][mA][mA][mA] | |
| | strand | C16 | [mG][mC][mCs][mAs][mA] | |
| UGT8- | Modified | Blunt- | [mCs][mA][mA][mU][mU][mA][mU][fG][fU][fU] | 861 |
| 277 | 20 mer sense | End | [fU][mG][ademA- | |
| | strand | P13 | C16][mA][mA][mG][mC][mCs][mAs][mA] | |
| | | C16 | | |
| UGT8- | Modified | Blunt- | [mCs][mA][mA][mU][mU][mA][mU][fG][fU][fU] | 862 |
| 277 | 20 mer sense | End | [fU][mG][mA][ademA- | |
| | strand | P14 | C16][mA][mG][mC][mCs][mAs][mA] | |
| | | C16 | | |
| UGT8- | Modified | Blunt- | [mCs][mA][mA][mU][mU][mA][mU][fG][fU][fU] | 863 |
| 277 | 20 mer sense | End | [fU][mG][mA][mA][ademA- | |
| | strand | P15 | C16][mG][mC][mCs][mAs][mA] | |
| | | C16 | | |
| UGT8- | Modified | Blunt- | [mCs][mA][mA][mU][mU][mA][mU][fG][fU][fU] | 864 |
| 277 | 20 mer sense | End | [fU][mG][mA][mA][mA][mG][mC][mCs][ademAs- | |
| | strand | P19 | C16][mA] | |
| | | C16 | | |
| UGT8- | Modified | Blunt- | [mCs][mA][mA][mU][mU][mA][mU][fG][fU][fU] | 865 |
| 277 | 20 mer sense | End | [fU][mG][mA][mA][mA][mG][mC][mCs][mAs] | |
| | strand | P20 | [ademA-C16] | |
| | | C16 | | |
| Stem- | Stem-loop | N/A | GCAGCCGAAAGGCUGC | 866 |
| loop | | | | |
| UGT8- | Modified | Blunt- | [ademCs- | 867 |
| 277 | 20 mer sense | End P1 | C16][mA][mA][mU][mU][mA][mU][fG][fU][fU] | |
| | strand | C16 | [fU][mG][mA][mA][mA][mG][mC][mCs][mAs] | |
| | | | [mA] | |
| UGT8- | Modified | Blunt- | [mCs][mA][mA][mU][ademU- | 868 |
| 277 | 20 mer sense | End P5 | C16][mA][mU][fG][fU][fU][fU][mG][mA][mA] | |
| | strand | C16 | [mA][mG][mC][mCs][mAs][mA] | |
| UGT8- | Modified | Blunt- | [mCs][mA][mA][mU][mU][mA][ademU- | 869 |
| 277 | 20 mer sense | End P7 | C16][fG][fU][fU][fU][mG][mA][mA][mA][mG] | |
| | strand | C16 | [mC][mCs][mAs][mA] | |
| UGT8- | Modified | Blunt- | [mCs][mA][mA][mU][mU][mA][mU][fG][ademU- | 870 |
| 277 | 20 mer sense | End P9 | C16][fU][fU][mG][mA][mA][mA][mG][mC][mCs | |
| | strand | C16 | ][mAs][mA] | |
| UGT8- | Modified | Blunt- | [mCs][mA][mA][mU][mU][mA][mU][fG][fU][fU] | 871 |
| 277 | 20 mer sense | End | [ademU- | |
| | strand | P11 | C16][mG][mA][mA][mA][mG][mC][mCs][mAs] | |
| | | C16 | [mA] | |
| UGT8- | Modified | Blunt- | [mCs][mA][mA][mU][mU][mA][mU][fG][fU][fU] | 872 |
| 277 | 20 mer sense | End | [fU][mG][mA][mA][mA][mG][ademC- | |
| | strand | P17 | C16][mCs][mAs][mA] | |
| | | C16 | | |

1.-381. (canceled)

382. A double-stranded oligonucleotide comprising an antisense strand of about 20-22 nucleotides in length and a sense strand of about 18-20 nucleotides in length, wherein the antisense and sense strands form a duplex region of about 18-20 base pairs, wherein the antisense strand comprises an orientation of 5′ to 3′, wherein the antisense strand comprises a 3′ overhang of at least one nucleotide, wherein the antisense strand comprises a region of complementarity to a mRNA target sequence, and wherein the sense strand comprises at least one lipid moiety conjugated to a nucleotide on the sense strand.

383. A double-stranded oligonucleotide comprising an antisense strand of 18-22 nucleotides in length and a sense strand of about 16-20 nucleotides in length, wherein the antisense and sense strands form a duplex region of at least 16-20 base pairs, wherein the antisense strand comprises an orientation of 5′ to 3′, wherein the antisense strand comprises a 5′ overhang of at least one nucleotide, wherein the antisense strand comprises a 3′ overhang of at least one nucleotide, wherein the antisense strand further comprises a region of complementarity to a mRNA target sequence, and wherein the sense strand further comprises at least one lipid moiety conjugated to a nucleotide on the sense strand.

384. The oligonucleotide of claim 382, wherein the lipid moiety is selected from:
a C8-C30 hydrocarbon chain,

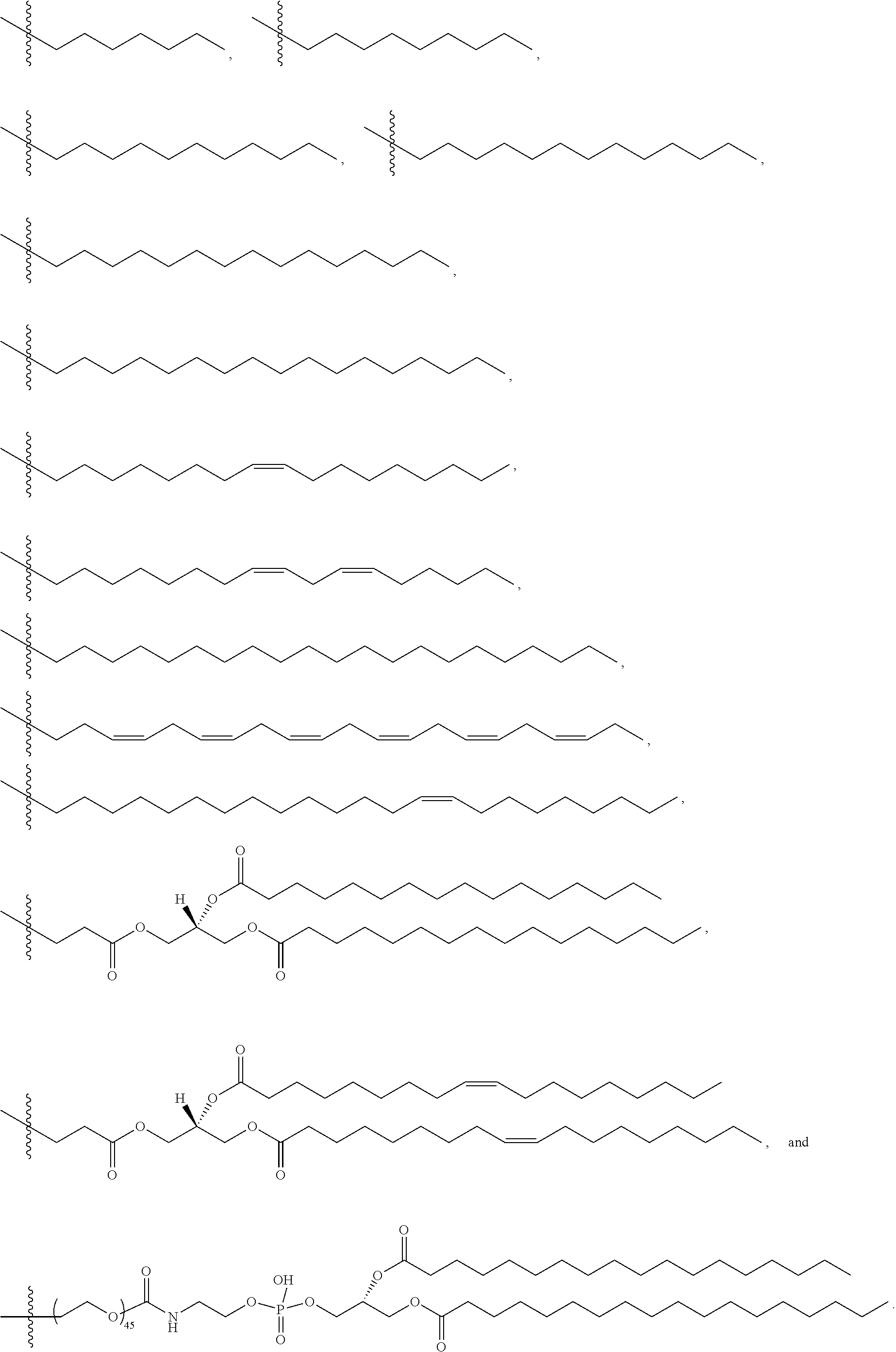

385. The oligonucleotide claim 382, wherein the lipid moiety is conjugated to at least one of the following positions:
a) the 5′ terminal nucleotide of the sense strand;
b) position 9 of the sense strand; and
c) the 3′ terminal nucleotide of the sense strand,
wherein the positions of the sense strand are numbered starting at the 5′ end to the 3′ end.

386. The oligonucleotide of claim 382, wherein the oligonucleotide comprises at least one modified nucleotide selected from 2′-aminoethyl, 2′-fluoro, 2′-O-methyl, 2′-O-methoxyethyl, or 2′-deoxy-2′-fluoro-β-d-arabinonucleic acid.

387. The oligonucleotide of claim 382, wherein the oligonucleotide comprises at least one modified internucleotide linkage.

388. The oligonucleotide of claim 382, wherein the sense strand comprises at least one Tₘ-increasing nucleotide.

389. The oligonucleotide of claim 382, wherein the lipid moiety is conjugated to the 2′ carbon of the ribose ring of the nucleotide.

390. The oligonucleotide of claim 382, wherein the antisense strand comprises a 4′-O-monomethylphosphonate-2′-O-methyl uridine at the 5′ terminus.

391. The oligonucleotide of claim 382, wherein the 4′-carbon of the sugar of the 5′-nucleotide of the antisense strand comprises a phosphate analog, selected from oxymethyl phosphonate, vinyl phosphonate, or malonyl phosphonate.

392. The oligonucleotide of claim 382, wherein the region of complementarity is fully complementary to the mRNA target sequence.

393. The oligonucleotide of claim 382, wherein the region of complementarity is partially complementary to the mRNA target sequence.

394. The oligonucleotide of claim 393, wherein the region of complementarity comprises no more than four mismatches to the mRNA target sequence.

395. The oligonucleotide of claim 382, wherein the mRNA target sequence is a central nervous system (CNS) target sequence, a neuronal mRNA target sequence, or an ocular mRNA target sequence.

396. The oligonucleotide of claim 382, wherein the mRNA target sequence is a liver mRNA target sequence, a hepatocyte mRNA target sequence, a liver macrophage mRNA target sequence, or a liver sinusoidal endothelial cell mRNA target sequence.

397. A pharmaceutical composition comprising the oligonucleotide of claim 382, and a pharmaceutically acceptable carrier, delivery agent or excipient.

398. A method for treating a subject having a disease, disorder or condition associated with expression of a target mRNA, the method comprising administering to the subject a therapeutically effective amount of the oligonucleotide of claim 382.

399. A method of reducing expression of a target mRNA in a subject, comprising administering to the subject the oligonucleotide of claim 382.

400. A method for treating a subject having a disease, disorder or condition associated with expression of an mRNA of the central nervous system, an mRNA of the liver, or an ocular mRNA, the method comprising administering to the subject a therapeutically effective amount of the oligonucleotide of claim 382.

401. The oligonucleotide of claim 383, wherein the antisense strand is 20 nucleotides in length and the sense strand is 16 nucleotides in length.

402. The oligonucleotide of claim 383, wherein the antisense strand is 22 nucleotides in length and the sense strand is 18 nucleotides in length.
